(19) United States
(12) Patent Application Publication
(10) Pub. No.: US 2026/0263639 A1
(43) Pub. Date: Sep. 10, 2026

(54) COMPOSITIONS COMPRISING POLYRIBONUCLEOTIDES AND USES THEREOF

(71) Applicant: Flagship Pioneering Innovations VI, LLC, Cambridge, MA (US)

(72) Inventors: Revital BRONSTEIN, Sharon, MA (US); Elissa Magdalene HOBERT, Cambridge, MA (US); Teymur Spartakovich KAZAKOV, Plymouth, MA (US); Ki Young PAKE, Brighton, MA (US)

(21) Appl. No.: 19/165,354

(22) PCT Filed: Mar. 15, 2024
(86) PCT No.: PCT/US2024/020314

§ 371 Date: Sep. 15, 2025

Related U.S. Application Data
(60) Provisional application No. 63/452,419, filed on Mar. 15, 2023.

(51) Int. Cl. A61K 48/00; A61K 31/711; C12N 15/68; C12N 15/85

(52) U.S. Cl. CPC A61K 48/0058; A61K 31/711; A61K 48/0066; C12N 15/68; C12N 15/85; C12N 2310/12; C12N 2310/532; C12N 2830/34; C12N 2830/50; C12N 2840/203; C12N 2840/44

(57) ABSTRACT

The present disclosure features compositions including polyribonucleotides having one or more expression augmenting elements or spacer elements. The polyribonucleotides described herein may increase the stability of the polyribonucleotide and/or may increase the expression of a polynucleotide cargo encoded by the polyribonucleotide.

### BACKGROUND

Polyribonucleotides are susceptible to degradation in the cell, preventing them from persisting and potentially from expressing a therapeutic polypeptide they encode. This represents a major obstacle to using polyribonucleotides as a potential therapeutic. Thus, there remains a need for methods of increasing the stability of polyribonucleotides and enhancing the translation of their polypeptide cargos.

### SUMMARY

In an aspect, the disclosure provides a circular polyribonucleotide comprising, in the following order from 5′ to 3′: (a) a first post-circularization element; (b) one or more expression augmenting element; (c) a polyribonucleotide cargo; and (d) a second post-circularization element; wherein the first post-circularization element and the second post-circularization element together form a circularization junction.

In another aspect, the disclosure provides circular polyribonucleotide comprising, in the following order from 5′ to 3′: (a) a first post-circularization element; (b) a polyribonucleotide cargo; (c) one or more expression augmenting element; and (d) a second post-circularization element; wherein the first post-circularization element and the second post-circularization element together form a circularization junction.

In some embodiments, the one or more expression augmenting element comprises a fusion of a translation enhancer and a spacer element comprising a polyA region. In some embodiments, the spacer element comprises from 5 to 200 ribonucleotides in length (e.g., from 5 to 150, 5 to 100, 5 to 50, 5 to 20, 20 to 200, 50 to 200, 100 to 200, 150 to 200, and 50 to 150 ribonucleotides in length). In some embodiments, the one or more expression augmenting element comprises a translation enhancer. In some embodiments, the translation enhancer is from a plant virus. In some embodiments, the translation enhancer is from a mammalian virus. In some embodiments, the translation enhancer is a human translation enhancer. In some embodiments, the translation enhancer is selected from the group comprising a BYDV like-element (BTE), a translation enhancer element (TED), a PMV/PEMV-like translation enhancer (PTE), an I-shaped structure (ISS), a Y-shaped structure (YSS), a t-shaped structure (TSS), dumbbell shaped structure, viral RNA UTRs (including Dengue, West Nile, Zika, Rotavirus), EMCV, CVB3, hepatitis B virus posttranscriptional regulatory element, human genomic fragments, a histone mRNA sequence, a cyclin D mRNA sequence, or an elF4g aptamer sequence. In some embodiments, the translation enhancer binds to elF4g.

In some embodiments, the translation enhancer comprises between 50 and 2000 (e.g., 50 to 1500, 50 to 1000, 50 to 500, 50 to 100, 50 to 1250, 50 to 1250, 50 to 750, 100 to 2000, 500 to 2000, 750 to 2000, 1000 to 2000, 1250 to 2000, 1500 to 2000, 1750 to 2000, 100 to 1000, and 500 to 1500) ribonucleotides. In some embodiments, the translation enhancer comprises between 60 and 800 (e.g., 60 to 700, 60 to 600, 60 to 500, 60 to 400, 60 to 400, 60 to 300, 60 to 300, 60 to 200, 60 to 100, 100 to 800, 200 to 800, 300 to 800, 400 to 800, 500 to 800, 600 to 800, 700 to 800, and 200 to 500) ribonucleotides.

In some embodiments, the circular polyribonucleotide exhibits at least 2-fold greater stability in comparison to a polyribonucleotide without a translation enhancer. In some embodiments, the circular polyribonucleotide may be detected for at least 2-fold longer in comparison to a polyribonucleotide without a translation enhancer after the circular polyribonucleotide is administered to a subject. In some embodiments, the circular polyribonucleotide comprises a polynucleotide cargo encodes a polypeptide, wherein the polypeptide has at least 4-fold greater expression in comparison to a polyribonucleotide without a translation enhancer.

In some embodiments, the one or more expression augmenting element comprises a fusion of a stability element and a spacer element comprising a polyA region. In some embodiments, the spacer element comprises from 5 to 200 ribonucleotides in length (e.g., from 5 to 150, 5 to 100, 5 to 50, 5 to 20, 20 to 200, 50 to 200, 100 to 200, 150 to 200, and 50 to 150 ribonucleotides in length). In some embodiments, the expression augmenting element comprises a stability element. In some embodiments, the stability element is an untranslated region (UTR). In some embodiments, the stability element is a 3′ UTR. In some embodiments, the stability element is a 5′ UTR. In some embodiments, the UTR is from a gene encoding a translocation associated membrane protein 1 (TRAM1), a transmembrane p24 trafficking protein 2 (TMED2), a vesicle associated membrane protein 3 (VAMP3), a CXXC repeat containing interactor of PDZ3 domain (CRIP), an adaptor related protein complex 2 subunit alpha 2 (AP2A2), a proteasome 26S subunit, a non-ATPase 5 (PSMD5), glutathione peroxidase 4 (GPX4), or a protein kinase AMP-activated non-catalytic subunit beta 1 (PRKAB1). In some embodiments, the UTR is a UTR from a human beta actin, DDB2, TP5313, FcIgG, LSP1, AES, DRB4, or a mitochondrially encoded 12S rRNA. In some embodiments, the stability element comprises between 50 and 2000 (e.g., 50 to 1500, 50 to 1000, 50 to 500, 50 to 100, 50 to 1250, 50 to 1250, 50 to 750, 100 to 2000, 500 to 2000, 750 to 2000, 1000 to 2000, 1250 to 2000, 1500 to 2000, 1750 to 2000, 100 to 1000, and 500 to 1500) ribonucleotides. In some embodiments, the stability element comprises between 60 and 800 (e.g., 60 to 700, 60 to 600, 60 to 500, 60 to 400, 60 to 400, 60 to 300, 60 to 300, 60 to 200, 60 to 100, 100 to 800, 200 to 800, 300 to 800, 400 to 800, 500 to 800, 600 to 800, 700 to 800, and 200 to 500) ribonucleotides. In some embodiments, the circular polyribonucleotide exhibits at least 2-fold greater stability in comparison to a polyribonucleotide without a stability element. In some embodiments, the circular polyribonucleotide may be detected for at least 2-fold longer in comparison to a polyribonucleotide without a stability element after the circular polyribonucleotide is administered to a subject. In some embodiments, the polynucleotide cargo encodes a polypeptide, wherein the polypeptide has at least 2-fold greater expression in comparison to a polyribonucleotide without a stability element.

In some embodiments, the circular polyribonucleotide further comprises one or more spacer elements comprising a polyA region. In some embodiments, the spacer element has a length of between 50 to 500 (e.g., 50 to 450, 50 to 400, 50 to 350, 50 to 300, 50 to 250, 50 to 200, 50 to 150, 50 to 100, 50 to 80, 55 to 500, 60 to 500, 70 to 500, 80 to 500, 90 to 500, 110 to 500, 115 to 500, 120 to 500, 150 to 500, 200 to 500, 250 to 500, 300 to 500, 350 to 500, 400 to 500, 450 to 500, 50 to 300, 50 to 200, 60 to 300, and 60 to 200) ribonucleotides. In some embodiments, the spacer element has a length of between 100 to 500 (e.g., 100 to 450, 100 to 400, 100 to 350, 100 to 300, 100 to 250, 100 to 200, 100 to 150, 110 to 500, 115 to 500, 120 to 500, 150 to 500, 200 to 500, 250 to 500, 300 to 500, 350 to 500, 400 to 500, 450 to 500, 100 to 300, 100 to 200, 110 to 300, and 110 to 200) ribonucleotides. In some embodiments, the spacer element has a length of between 110 to 500 (e.g., 110 to 450, 110 to 400, 110 to 350, 110 to 300, 110 to 250, 110 to 200, 110 to 150, 150 to 500, 200 to 500, 250 to 500, 300 to 500, 350 to 500, 400 to 500, and 450 to 500) ribonucleotides. In some embodiments, the spacer element has a length of between 120 to 500 (e.g., 120 to 450, 120 to 400, 120 to 350, 120 to 300, 120 to 250, 120 to 200, 120 to 150, 150 to 500, 200 to 500, 250 to 500, 300 to 500, 350 to 500, 400 to 500, and 450 to 500) ribonucleotides. In some embodiments, the spacer element has a length of between 100 to 300 (e.g., 100 to 280, 100 to 260, 100 to 240, 100 to 220, 100 to 200, 100 to 180, 100 to 160, 100 to 140, 100 to 120, 110 to 300, 115 to 300, 120 to 300, 140 to 300, 160 to 300, 180 to 300, 200 to 300, 220 to 300, 240 to 300, 260 to 300, 110 to 200, and 280 to 300) ribonucleotides. In some embodiments, the spacer has a length of between 10 and 200 (e.g., between 10 and 175, 10 and 150, 10 and 125, 10 and 100, 10 and 75, 10 and 50, 10 and 25, 25 and 200, 50 and 200, 75 and 200, 100 and 200, 150 and 200, 175 and 200, and 25 and 100) ribonucleotides. In some embodiments, the spacer element has a length of about 120 ribonucleotides. In some embodiments, the spacer element has a length of about 50 ribonucleotides.

In another aspect, the disclosure provides a circular polyribonucleotide comprising, in the following order from 5′ to 3′: (a) a first post-circularization element; (b) a first expression augmenting element; (c) a polyribonucleotide cargo; (d) a second expression augmenting element; and (e) a second post-circularization element; wherein the first post-circularization element and the second post-circularization element together form a circularization junction. In some embodiments, the first expression augmenting element comprises a fusion of a translation enhancer and a spacer element. In some embodiments, the spacer element comprises from 5 to 200 ribonucleotides in length (e.g., from 5 to 150, 5 to 100, 5 to 50, 5 to 20, 20 to 200, 50 to 200, 100 to 200, 150 to 200, and 50 to 150 ribonucleotides in length). In some embodiments, the second expression augmenting element comprises a fusion of a translation enhancer and a spacer element. In some embodiments, the spacer element comprises from 5 to 200 ribonucleotides in length (e.g., from 5 to 150, 5 to 100, 5 to 50, 5 to 20, 20 to 200, 50 to 200, 100 to 200, 150 to 200, and 50 to 150 ribonucleotides in length). In some embodiments, the first expression augmenting element comprises a translation enhancer. In some embodiments, the second expression augmenting element comprises a translation enhancer. In some embodiments, the translation enhancer is from a plant virus. In some embodiments, the translation enhancer is from a mammalian virus. In some embodiments, the translation enhancer is a human translation enhancer. In some embodiments, the translation enhancer is selected from the group comprising a BTE, a TED, a PTE, an ISS, a YSS, a TSS, dumbbell shaped structure, viral RNA UTRs (including Dengue, West Nile, Zika, Rotavirus), EMCV, CVB3, hepatitis B virus posttranscriptional regulatory element, human genomic fragments, a histone mRNA sequence, a cyclin D mRNA sequence, or an elF4g aptamer sequence. In some embodiments, the translation enhancer binds to elF4g. In some embodiments, the translation enhancer comprises between 50 and 2000 (e.g., 50 to 1500, 50 to 1000, 50 to 500, 50 to 100, 50 to 1250, 50 to 1250, 50 to 750, 100 to 2000, 500 to 2000, 750 to 2000, 1000 to 2000, 1250 to 2000, 1500 to 2000, 1750 to 2000, 100 to 1000, and 500 to 1500) ribonucleotides. In some embodiments, the translation enhancer comprises between 60 and 800 (e.g., 60 to 700, 60 to 600, 60 to 500, 60 to 400, 60 to 400, 60 to 300, 60 to 300, 60 to 200, 60 to 100, 100 to 800, 200 to 800, 300 to 800, 400 to 800, 500 to 800, 600 to 800, 700 to 800, and 200 to 500) ribonucleotides. In some embodiments, the circular polyribonucleotide exhibits at least 2-fold greater stability in comparison to a polyribonucleotide without a translation enhancer. In some embodiments, the circular polyribonucleotide may be detected for at least 2-fold longer in comparison to a polyribonucleotide without a translation enhancer after the circular polyribonucleotide is administered to a subject. In some embodiments, the circular polyribonucleotide comprises a polynucleotide cargo encoding a polypeptide, wherein the polypeptide has at least 4-fold greater expression in comparison to a polyribonucleotide without a translation enhancer.

In some embodiments, the first expression augmenting element comprises a fusion of a stability element and a spacer element comprising a polyA region. In some embodiments, the spacer element comprises from 5 to 200 ribonucleotides in length (e.g., from 5 to 150, 5 to 100, 5 to 50, 5 to 20, 20 to 200, 50 to 200, 100 to 200, 150 to 200, and 50 to 150 ribonucleotides in length). In some embodiments, the first expression augmenting element comprises a stability element. In some embodiments, the spacer element comprises from 5 to 200 ribonucleotides in length (e.g., from 5 to 150, 5 to 100, 5 to 50, 5 to 20, 20 to 200, 50 to 200, 100 to 200, 150 to 200, and 50 to 150 ribonucleotides in length). In some embodiments, the second expression augmenting element comprises a stability element. In some embodiments, the stability element is a UTR. In some embodiments, the UTR is from a gene encoding a TRAM1, a TMED2, a VAMP3, a CRIP, an AP2A2, a proteasome 26S subunit, a PSMD5, a GPX4, or a PRKAB1. In some embodiments, the UTR is a UTR from a human beta actin, DDB2, TP5313, FcIgG, LSP1, AES, DRB4, or a mitochondrially encoded 12S rRNA. In some embodiments, the stability element is a 3′ UTR. In some embodiments, the stability element is a 5′ UTR. In some embodiments, the stability element comprises between 50 and 2000 (e.g., 50 to 1500, 50 to 1000, 50 to 500, 50 to 100, 50 to 1250, 50 to 1250, 50 to 750, 100 to 2000, 500 to 2000, 750 to 2000, 1000 to 2000, 1250 to 2000, 1500 to 2000, 1750 to 2000, 100 to 1000, and 500 to 1500) ribonucleotides. In some embodiments, the stability element comprises between 60 and 800 (e.g., 60 to 700, 60 to 600, 60 to 500, 60 to 400, 60 to 400, 60 to 300, 60 to 300, 60 to 200, 60 to 100, 100 to 800, 200 to 800, 300 to 800, 400 to 800, 500 to 800, 600 to 800, 700 to 800, and 200 to 500) ribonucleotides. In some embodiments, the circular polyribonucleotide exhibits at least 2-fold greater stability in comparison to a polyribonucleotide without a stability element. In some embodiments, the circular polyribonucleotide may be detected for at least 2-fold longer in comparison to a polyribonucleotide without a stability element after the circular polyribonucleotide is administered to a subject. In some embodiments, the polynucleotide cargo encodes a polypeptide, wherein the polypeptide has at least 2-fold greater expression in comparison to a polyribonucleotide without a stability element.

In some embodiments, the circular polyribonucleotide further comprises one or more spacer elements comprising a polyA region. In some embodiments, the spacer element has a length of between 100 to 500 (e.g., 100 to 450, 100 to 400, 100 to 350, 100 to 300, 100 to 250, 100 to 200, 100 to 150, 120 to 500, 150 to 500, 200 to 500, 250 to 500, 300 to 500, 350 to 500, 400 to 500, and 450 to 500) ribonucleotides. In some embodiments, the spacer element has a length of between 120 to 500 (e.g., 120 to 450, 120 to 400, 120 to 350, 120 to 300, 120 to 250, 120 to 200, 120 to 150, 150 to 500, 200 to 500, 250 to 500, 300 to 500, 350 to 500, 400 to 500, and 450 to 500) ribonucleotides. In some embodiments, the spacer element has a length of between 100 to 300 (e.g., 100 to 280, 100 to 260, 100 to 240, 100 to 220, 100 to 200, 100 to 180, 100 to 160, 100 to 140, 100 to 120, 120 to 300, 140 to 300, 160 to 300, 180 to 300, 200 to 300, 220 to 300, 240 to 300, 260 to 300, and 280 to 300) ribonucleotides.

In another aspect, the disclosure provides a circular polyribonucleotide comprising, in the following order from 5′ to 3′: (a) a first post-circularization element; (b) a spacer element; (c) a polyribonucleotide cargo; (d) an expression augmenting element; and (e) a second post-circularization element and wherein the first post-circularization element and the second post-circularization element together form a circularization junction.

In another aspect, the disclosure provides a circular polyribonucleotide comprising, in the following order from 5′ to 3′: (a) a first post-circularization element; (b) an expression augmenting element; (c) a polyribonucleotide cargo; (d) a spacer element; and (e) a second post-circularization element and wherein the first post-circularization element and the second post-circularization element together form a circularization junction.

In another aspect, the disclosure provides a circular polyribonucleotide comprising, in the following order from 5′ to 3′: (a) a first post-circularization element; (b) a first expression augmenting element; (c) a polyribonucleotide cargo; (d) a second expression augmenting element; and (e) a second post-circularization element and wherein the first post-circularization element and the second post-circularization element together form a circularization junction.

In another aspect, the disclosure provides a circular polyribonucleotide comprising, in the following order from 5′ to 3′: (a) a first post-circularization element; (b) a first spacer element having a length of at least 100 ribonucleotides; (c) a polyribonucleotide cargo; (d) a second spacer element; and

(e) a second post-circularization element; and wherein the first post-circularization element and the second post-circularization element together form a circularization junction.

In another aspect, the disclosure provides a circular polyribonucleotide comprising, in the following order from 5′ to 3′: (a) a first post-circularization element; (b) a first spacer element; (c) a polyribonucleotide cargo; (d) a second spacer element having a length of at least 100 ribonucleotides; and (e) a second post-circularization element; and wherein the first post-circularization element and the second post-circularization element together form a circularization junction.

In some embodiments, the first spacer element has a length of between 50 to 500 (e.g., 50 to 450, 50 to 400, 50 to 350, 50 to 300, 50 to 250, 50 to 200, 50 to 150, 50 to 100, 50 to 80, 55 to 500, 60 to 500, 70 to 500, 80 to 500, 90 to 500, 110 to 500, 115 to 500, 120 to 500, 150 to 500, 200 to 500, 250 to 500, 300 to 500, 350 to 500, 400 to 500, 450 to 500, 50 to 300, 50 to 200, 60 to 300, and 60 to 200) ribonucleotides. In some embodiments, the first spacer element has a length of between 100 to 500 (e.g., 100 to 450, 100 to 400, 100 to 350, 100 to 300, 100 to 250, 100 to 200, 100 to 150, 110 to 450, 110 to 400, 110 to 350, 110 to 300, 110 to 250, 110 to 200, 110 to 150, 120 to 500, 150 to 500, 200 to 500, 250 to 500, 300 to 500, 350 to 500, 400 to 500, and 450 to 500) ribonucleotides. In some embodiments, the second spacer element has a length of between 50 to 500 (e.g., 50 to 450, 50 to 400, 50 to 350, 50 to 300, 50 to 250, 50 to 200, 50 to 150, 50 to 100, 50 to 80, 55 to 500, 60 to 500, 70 to 500, 80 to 500, 90 to 500, 110 to 500, 115 to 500, 120 to 500, 150 to 500, 200 to 500, 250 to 500, 300 to 500, 350 to 500, 400 to 500, 450 to 500, 50 to 300, 50 to 200, 60 to 300, and 60 to 200) ribonucleotides. In some embodiments, the second spacer element has a length of between 100 to 500 (e.g., 100 to 450, 100 to 400, 100 to 350, 100 to 300, 100 to 250, 100 to 200, 100 to 150, 110 to 450, 110 to 400, 110 to 350, 110 to 300, 110 to 250, 110 to 200, 110 to 150, 120 to 500, 150 to 500, 200 to 500, 250 to 500, 300 to 500, 350 to 500, 400 to 500, and 450 to 500) ribonucleotides. In some embodiments, the first spacer element and the second spacer element each comprise 110 to 500 (e.g., 110 to 450, 110 to 400, 110 to 350, 110 to 300, 110 to 250, 110 to 200, 110 to 150, 150 to 500, 200 to 500, 250 to 500, 300 to 500, 350 to 500, 400 to 500, and 450 to 500) ribonucleotides. In some embodiments, the first spacer element and the second spacer element each comprise 120 to 500 (e.g., 120 to 450, 120 to 400, 120 to 350, 120 to 300, 120 to 250, 120 to 200, 120 to 150, 150 to 500, 200 to 500, 250 to 500, 300 to 500, 350 to 500, 400 to 500, and 450 to 500) ribonucleotides. In some embodiments, the first spacer element and the second spacer element each have a length of 150 to 500 (e.g., 150 to 450, 150 to 400, 150 to 350, 150 and 300, 150 and 250, 150 and 200, 200 500, 250 and 500, 300 and 500, 350 and 500, 400 and 500, or 450 and 500) ribonucleotides. In some embodiments, the first spacer element and the second spacer element each have a length of 200 to 500 (e.g., 200 to 450, 200 to 400, 200 to 350, 200 to 300, 200 to 250, 250 to 500, 300 to 500, 350 to 500, 400 to 500, or 450 to 500) ribonucleotides. In some embodiments, the first spacer element and the second spacer element each have a length of 100 to 300 (e.g., 100 to 280, 100 to 260, 100 to 240, 100 to 220, 100 to 200, 100 to 180, 100 to 160, 100 to 140, 100 to 120, 120 to 300, 140 to 300, 160 to 300, 180 to 300, 200 to 300, 220 to 300, 240 to 300, 260 to 300, and 280 to 300) ribonucleotides. In some embodiments, the first spacer element and the second spacer element each have a length of 100 to 200 (e.g., 100 to 190, 100 to 180, 100 to 170, 100 to 160, 100 to 150, 100 to 140, 100 to 130, 100 to 120, 100 to 110, 110 to 200, 120 to 200, 130 to 200, 140 to 200, 150 to 200, 160 to 200, 170 to 200, 180 to 200, and 90 to 200) ribonucleotides. In some embodiments, the first spacer element and the second spacer element each have a length of 110 to 300 (e.g., 110 to 280, 110 to 260, 110 to 240, 110 to 220, 110 to 200, 110 to 180, 110 to 160, 110 to 140, 140 to 300, 160 to 300, 180 to 300, 200 to 300, 220 to 300, 240 to 300, 260 to 300, and 280 to 300) ribonucleotides. In some embodiments, the first spacer element and the second spacer element each have a length of 120 to 300 (e.g., 120 to 280, 120 to 260, 120 to 240, 120 to 220, 120 to 200, 120 to 180, 120 to 160, 120 to 140, 140 to 300, 160 to 300, 180 to 300, 200 to 300, 220 to 300, 240 to 300, 260 to 300, and 280 to 300) ribonucleotides. In some embodiments, the first spacer element and the second spacer element each have a length of 150 to 300 (e.g., 150 to 290, 150 to 280, 150 to 270, 150 to 260, 150 to 250, 150 to 240, 150 to 230, 150 to 220, 150 to 210, 150 to 200, 150 to 190, 150 to 180, 150 to 170, 150 to 160, 160 to 300, 170 to 300, 180 to 300, 190 to 300, 200 to 300, 210 to 300, 220 to 300, 230 to 300, 240 to 300, 250 to 300, 260 to 300, 270 to 300, 280 to 300, and 290 to 300) ribonucleotides.

In some embodiments, the difference between the length of the first spacer element and the length of the second spacer element is 0 to 100 (e.g., 0 and 10, 0 and 20, 0 and 30, 0, and 40, 0 and 50, 0 to 60, 0 to 70, 0 to 80, 0 to 90, 10 to 100, 20 to 100, 30 to 100, 40 to 100, 50 to 100, 60 to 100, 70 to 100, 80 to 100, 90 to 100, 5 to 10, 5 to 20, 10 to 30 or 5 to 50) nucleotides. In some embodiments, the difference between the length of the first spacer element and the length of the second spacer element is 0 to 50 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50) nucleotides. In some embodiments, the first spacer element and the second spacer element are the same number of ribonucleotides in length.

In some embodiments, the first spacer or the second spacer element consists of: (i) a polyA region comprising 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, and 100%) adenosine residues; (ii) a polyAC region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, and 100%) adenosine or cytosine residues; (iii) a polyAU region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, and 100%) adenosine or uridine residues; or (iii) a polyAG region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, and 100%) adenosine or guanosine residues.

In some embodiments, the circular polyribonucleotide exhibits at least 2-fold greater stability in comparison to a polyribonucleotide without the first spacer element or the second spacer element. In some embodiments, the circular polyribonucleotide may be detected for at least 2-fold longer in comparison to a polyribonucleotide without the first spacer element or the second spacer element after the circular polyribonucleotide is administered to a subject. In some embodiments, the polynucleotide cargo encodes a polypeptide, wherein the polypeptide has at least 4-fold greater expression in comparison to a polyribonucleotide without the first spacer element or the second spacer element.

In some embodiments, the polyribonucleotide cargo comprises an expression sequence. In some embodiments, the polyribonucleotide cargo comprises an IRES operably linked an expression sequence. In some embodiments, the expression sequence further comprises a 3′ untranslated region or a 5′ untranslated region. In some embodiments, the expression sequence encodes a polypeptide.

In some embodiments, the polyribonucleotide comprises between 500 and 20,000 (e.g., between 500 to 15,000, 500 to 10,000, 500 to 5,000, 500 to 1,000, 1,000 to 20,000, 5,000 to 20,000, 10,000 to 20,000, and 15,000 to 20,000) ribonucleotides. In some embodiments, the polyribonucleotide comprises between 2,000 and 20,000 (e.g., 2,000 to 5,000, 2,000 to 7,500, 2,000 to 10,000, 2,000 to 12,500, 2,000 to 15,000, 2,000 to 17,500, 5,000 to 20,000, 7,500 to 20,000, 10,000 to 20,000, 12,500 to 20,000, 15,000 to 20,000, and 17,500 to 20,000) ribonucleotides.

In some embodiments, the circularization junction comprises a splice junction. In some embodiments, the first post-circularization element comprises a first exon fragment and the second post-circularization element comprises a second exon fragment, and wherein the first exon fragment and the second exon fragment are joined by the splice junction. In some embodiments, the circularization junction comprises an oligonucleotide splint that is hybridized to the first post-circularization element and to the second post-circularization element. In some embodiments, the oligonucleotide splint is a DNA splint or an RNA splint. In some embodiments, the first post-circularization element comprises a region that is capable of hybridizing to a first region of the oligonucleotide splint and the second post-circularization element comprises a region that is capable of hybridizing to a second region of the oligonucleotide splint.

In an aspect, the disclosure provides a linear polyribonucleotide comprising, in the following order from 5′ to 3′: (a) a first post-circularization element; (b) one or more expression augmenting element; (c) a polyribonucleotide cargo; and (d) a second post-circularization element; wherein the first post-circularization element and the second post-circularization element together form a circularization junction.

In an aspect, the disclosure provides a linear polyribonucleotide comprising, in the following order from 5′ to 3′: (a) a first post-circularization element; (b) a polyribonucleotide cargo; (c) one or more expression augmenting element; and (d) a second post-circularization element; wherein the first post-circularization element and the second post-circularization element together form a circularization junction.

In some embodiments, the one or more expression augmenting element comprises a fusion of a translation enhancer and a spacer element comprising a polyA region. In some embodiments, the spacer element comprises from 5 to 200 ribonucleotides in length (e.g., from 5 to 150, 5 to 100, 5 to 50, 5 to 20, 20 to 200, 50 to 200, 100 to 200, 150 to 200, and 50 to 150 ribonucleotides in length). In some embodiments, the one or more expression augmenting element comprises a translation enhancer. In some embodiments, the translation enhancer is from a plant virus. In some embodiments, the translation enhancer is from a mammalian virus. In some embodiments, the translation enhancer is a human translation enhancer. In some embodiments, the translation enhancer is selected from the group comprising a BTE, a TED, a PTE, an ISS, a YSS, a TSS, dumbbell shaped structure, viral RNA UTRs (including Dengue, West Nile, Zika, Rotavirus), EMCV, CVB3, hepatitis B virus posttranscriptional regulatory element, human genomic fragments, a histone mRNA sequence, a cyclin D mRNA sequence, or an elF4g aptamer sequence. In some embodiments, the translation enhancer binds to elF4g. In some embodiments, the translation enhancer comprises between 50 and 2000 (e.g., 50 to 1500, 50 to 1000, 50 to 500, 50 to 100, 50 to 1250, 50 to 1250, 50 to 750, 100 to 2000, 500 to 2000, 750 to 2000, 1000 to 2000, 1250 to 2000, 1500 to 2000, 1750 to 2000, 100 to 1000, and 500 to 1500) ribonucleotides. In some embodiments, the translation enhancer comprises between 60 and 800 (e.g., 60 to 700, 60 to 600, 60 to 500, 60 to 400, 60 to 400, 60 to 300, 60 to 300, 60 to 200, 60 to 100, 100 to 800, 200 to 800, 300 to 800, 400 to 800, 500 to 800, 600 to 800, 700 to 800, and 200 to 500) ribonucleotides.

In some embodiments, the linear polyribonucleotide exhibits at least 2-fold greater stability in comparison to a polyribonucleotide without a translation enhancer. In some embodiments, the linear polyribonucleotide may be detected for at least 2-fold longer in comparison to a polyribonucleotide without a translation enhancer after the linear polyribonucleotide is administered to a subject. In some embodiments, the linear polyribonucleotide is polynucleotide cargo encodes a polypeptide, wherein the polypeptide has at least 4-fold greater expression in comparison to a polyribonucleotide without a translation enhancer.

In some embodiments, the one or more expression augmenting element comprises a fusion of a stability element and a spacer element comprising a polyA region. In some embodiments, the spacer element comprises from 5 to 200 ribonucleotides in length (e.g., from 5 to 150, 5 to 100, 5 to 50, 5 to 20, 20 to 200, 50 to 200, 100 to 200, 150 to 200, and 50 to 150 ribonucleotides in length). In some embodiments, the expression augmenting element comprises a stability element. In some embodiments, the stability element is a UTR. In some embodiments, the UTR is from a gene encoding TRAM1, a TMED2, a VAMP3, a CRIP, an AP2A2, a proteasome 26S subunit, a PSMD5, a GPX4, or a PRKAB1. In some embodiments, the UTR is a UTR from a human beta actin, DDB2, TP5313, FcIgG, LSP1, AES, DRB4, or a mitochondrially encoded 12S rRNA. In some embodiments, the stability element is a 3′ UTR. In some embodiments, the stability element is a 5′ UTR.

In some embodiments, the stability element comprises between 50 and 2000 (e.g., 50 to 1500, 50 to 1000, 50 to 500, 50 to 100, 50 to 1250, 50 to 1250, 50 to 750, 100 to 2000, 500 to 2000, 750 to 2000, 1000 to 2000, 1250 to 2000, 1500 to 2000, 1750 to 2000, 100 to 1000, and 500 to 1500) ribonucleotides. In some embodiments, the stability element comprises between 60 and 800 (e.g., 60 to 700, 60 to 600, 60 to 500, 60 to 400, 60 to 400, 60 to 300, 60 to 300, 60 to 200, 60 to 100, 100 to 800, 200 to 800, 300 to 800, 400 to 800, 500 to 800, 600 to 800, 700 to 800, and 200 to 500) ribonucleotides.

In some embodiments, the linear polyribonucleotide exhibits at least 2-fold greater stability in comparison to a polyribonucleotide without a stability element. In some embodiments, the linear polyribonucleotide may be detected for at least 2-fold longer in comparison to a polyribonucleotide without a stability element after the linear polyribonucleotide is administered to a subject. In some embodiments, the polynucleotide cargo encodes a polypeptide, wherein the polypeptide has at least 2-fold greater expression in comparison to a polyribonucleotide without a stability element.

In some embodiments, the linear polyribonucleotide further comprises one or more spacer elements comprising a polyA region. In some embodiments, the spacer element has a length of between 100 to 500 (e.g., 100 to 450, 100 to 400, 100 to 350, 100 to 300, 100 to 250, 100 to 200, 100 to 150, 110 to 500, 120 to 500, 150 to 500, 200 to 500, 250 to 500, 300 to 500, 350 to 500, 400 to 500, and 450 to 500) ribonucleotides. In some embodiments, the spacer element has a length of between 110 to 500 (e.g., 110 to 450, 110 to 400, 110 to 350, 110 to 300, 110 to 250, 110 to 200, 110 to 150, 150 to 500, 200 to 500, 250 to 500, 300 to 500, 350 to 500, 400 to 500, and 450 to 500) ribonucleotides. In some embodiments, the spacer element has a length of between 120 to 500 (e.g., 120 to 450, 120 to 400, 120 to 350, 120 to 300, 120 to 250, 120 to 200, 120 to 150, 150 to 500, 200 to 500, 250 to 500, 300 to 500, 350 to 500, 400 to 500, and 450 to 500) ribonucleotides. In some embodiments, the spacer element has a length of between 100 to 300 (e.g., 100 to 280, 100 to 260, 100 to 240, 100 to 220, 100 to 200, 100 to 180, 100 to 160, 100 to 140, 100 to 120, 110 to 300, 120 to 300, 140 to 300, 160 to 300, 180 to 300, 200 to 300, 220 to 300, 240 to 300, 260 to 300, and 280 to 300) ribonucleotides.

In another aspect, the disclosure provides a linear polyribonucleotide comprising, in the following order from 5′ to 3′: (a) a first post-circularization element; (b) a first expression augmenting element; (c) a polyribonucleotide cargo; (d) a second expression augmenting element; and (e) a second post-circularization element; wherein the first post-circularization element and the second post-circularization element together form a circularization junction. In some embodiments, the first expression augmenting element comprises a fusion of a translation enhancer and a spacer element. In some embodiments, the spacer element comprises from 5 to 200 ribonucleotides in length (e.g., from 5 to 150, 5 to 100, 5 to 50, 5 to 20, 20 to 200, 50 to 200, 100 to 200, 150 to 200, and 50 to 150 ribonucleotides in length). In some embodiments, the second expression augmenting element comprises a fusion of a translation enhancer and a spacer element. In some embodiments, the spacer element comprises from 5 to 200 ribonucleotides in length (e.g., from 5 to 150, 5 to 100, 5 to 50, 5 to 20, 20 to 200, 50 to 200, 100 to 200, 150 to 200, and 50 to 150 ribonucleotides in length). In some embodiments, the first expression augmenting element comprises a translation enhancer. In some embodiments, the second expression augmenting element comprises a translation enhancer. In some embodiments, the translation enhancer is from a plant virus. In some embodiments, the translation enhancer is from a mammalian virus. In some embodiments, the translation enhancer is a human translation enhancer. In some embodiments, the translation enhancer is selected from the group comprising a BTE, a TED, a PTE, an ISS, a YSS, a TSS, dumbbell shaped structure, viral RNA UTRs (including Dengue, West Nile, Zika, Rotavirus), EMCV, CVB3, hepatitis B virus posttranscriptional regulatory element, human genomic fragments, a histone mRNA sequence, a cyclin D mRNA sequence, or an elF4g aptamer sequence. In some embodiments, the translation enhancer binds to elF4g.

In some embodiments, the translation enhancer comprises between 50 and 2000 (e.g., 50 to 1500, 50 to 1000, 50 to 500, 50 to 100, 50 to 1250, 50 to 1250, 50 to 750, 100 to 2000, 500 to 2000, 750 to 2000, 1000 to 2000, 1250 to 2000, 1500 to 2000, 1750 to 2000, 100 to 1000, and 500 to 1500) ribonucleotides. In some embodiments, the translation enhancer comprises between 60 and 800 (e.g., 60 to 700, 60 to 600, 60 to 500, 60 to 400, 60 to 400, 60 to 300, 60 to 300, 60 to 200, 60 to 100, 100 to 800, 200 to 800, 300 to 800, 400 to 800, 500 to 800, 600 to 800, 700 to 800, and 200 to 500) ribonucleotides.

In some embodiments, the linear polyribonucleotide exhibits at least 2-fold greater stability in comparison to a polyribonucleotide without a translation enhancer. In some embodiments, the linear polyribonucleotide may be detected for at least 2-fold longer in comparison to a polyribonucleotide without a translation enhancer after the linear polyribonucleotide is administered to a subject. In some embodiments, the linear polyribonucleotide is polynucleotide cargo encodes a polypeptide, wherein the polypeptide has at least 4-fold greater expression in comparison to a polyribonucleotide without a translation enhancer.

In some embodiments, the first expression augmenting element comprises a fusion of a stability element and a spacer element comprising a polyA region. In some embodiments, the spacer element comprises from 5 to 200 ribonucleotides in length (e.g., from 5 to 150, 5 to 100, 5 to 50, 5 to 20, 20 to 200, 50 to 200, 100 to 200, 150 to 200, and 50 to 150 ribonucleotides in length). In some embodiments, the first expression augmenting element comprises a stability element. In some embodiments, the second expression augmenting element comprises a fusion of a stability element and a spacer element. In some embodiments, the spacer element comprises from 5 to 200 ribonucleotides in length (e.g., from 5 to 150, 5 to 100, 5 to 50, 5 to 20, 20 to 200, 50 to 200, 100 to 200, 150 to 200, and 50 to 150 ribonucleotides in length). In some embodiments, the second expression augmenting element comprises a stability element. In some embodiments, the stability element is a UTR. In some embodiments, the UTR is from a gene encoding a TRAM1, a TMED2, a VAMP3, a CRIP, an AP2A2, a proteasome 26S subunit, a PSMD5, a GPX4, or a PRKAB1. In some embodiments, the UTR is a UTR from a human beta actin, DDB2, TP5313, FcIgG, LSP1, AES, DRB4, or a mitochondrially encoded 12S rRNA. In some embodiments, the stability element is a 3′ UTR. In some embodiments, the stability element is a 5′ UTR.

In some embodiments, the stability element comprises between 50 and 2000 (e.g., 50 to 1500, 50 to 1000, 50 to 500, 50 to 100, 50 to 1250, 50 to 1250, 50 to 750, 100 to 2000, 500 to 2000, 750 to 2000, 1000 to 2000, 1250 to 2000, 1500 to 2000, 1750 to 2000, 100 to 1000, and 500 to 1500) ribonucleotides. In some embodiments, the stability element comprises between 60 and 800 (e.g., 60 to 700, 60 to 600, 60 to 500, 60 to 400, 60 to 400, 60 to 300, 60 to 300, 60 to 200, 60 to 100, 100 to 800, 200 to 800, 300 to 800, 400 to 800, 500 to 800, 600 to 800, 700 to 800, and 200 to 500) ribonucleotides.

In some embodiments, the linear polyribonucleotide exhibits at least 2-fold greater stability in comparison to a polyribonucleotide without a stability element. In some embodiments, the linear polyribonucleotide may be detected for at least 2-fold longer in comparison to a polyribonucleotide without a stability element after the linear polyribonucleotide is administered to a subject. In some embodiments, the polynucleotide cargo encodes a polypeptide, wherein the polypeptide has at least 2-fold greater expression in comparison to a polyribonucleotide without a stability element.

In some embodiments, the linear polyribonucleotide further comprises one or more spacer elements comprising a polyA region. In some embodiments, the spacer element has a length of between 100 to 500 (e.g., 100 to 450, 100 to 400, 100 to 350, 100 to 300, 100 to 250, 100 to 200, 100 to 150, 120 to 500, 150 to 500, 200 to 500, 250 to 500, 300 to 500, 350 to 500, 400 to 500, and 450 to 500) ribonucleotides. In some embodiments, the spacer element has a length of between 120 to 500 (e.g., 120 to 450, 120 to 400, 120 to 350, 120 to 300, 120 to 250, 120 to 200, 120 to 150, 150 to 500, 200 to 500, 250 to 500, 300 to 500, 350 to 500, 400 to 500, and 450 to 500) ribonucleotides. In some embodiments, the spacer element has a length of between 100 to 300 (e.g., 100 to 280, 100 to 260, 100 to 240, 100 to 220, 100 to 200, 100 to 180, 100 to 160, 100 to 140, 100 to 120, 120 to 300, 140 to 300, 160 to 300, 180 to 300, 200 to 300, 220 to 300, 240 to 300, 260 to 300, and 280 to 300) ribonucleotides.

In another aspect, the disclosure provides a linear polyribonucleotide comprising, in the following order from 5′ to 3′: (a) a first post-circularization element; (b) a spacer element; (c) a polyribonucleotide cargo; (d) an expression augmenting element and (e) a second post-circularization element; and wherein the first post-circularization element and the second post-circularization element together form a circularization junction.

In another aspect, the disclosure provides a linear polyribonucleotide comprising, in the following order from 5′ to 3′: (a) a first post-circularization element; (b) an expression augmenting element; (c) a polyribonucleotide cargo; (d) a spacer element; and (e) a second post-circularization element, and wherein the first post-circularization element and the second post-circularization element together form a circularization junction.

In another aspect, the disclosure provides a linear polyribonucleotide comprising, in the following order from 5′ to 3′: (a) a first post-circularization element; (b) a first expression augmenting element; (c) a polyribonucleotide cargo; (d) a second expression augmenting element; and (e) a second post-circularization element, and wherein the first post-circularization element and the second post-circularization element together form a circularization junction.

In another aspect, the disclosure provides a linear polyribonucleotide comprising, in the following order from 5′ to 3′: (a) a first post-circularization element; (b) a first spacer element having a length of at least 100 ribonucleotides; (c) a polyribonucleotide cargo; (d) a second spacer element; and (e) a second post-circularization element; and wherein the first post-circularization element and the second post-circularization element together form a circularization junction.

In another aspect, the disclosure provides a linear polyribonucleotide comprising, in the following order from 5′ to 3′: (a) a first post-circularization element; (b) a first spacer element having a length of at least 100 ribonucleotides; (c) a polyribonucleotide cargo; (d) a second spacer element and (e) a second post-circularization element; and wherein the first post-circularization element and the second post-circularization element together form a circularization junction.

In another aspect, the disclosure provides a linear polyribonucleotide comprising, in the following order from 5′ to 3′: (a) a first post-circularization element; (b) a first spacer element; (c) a polyribonucleotide cargo; (d) a second spacer element having a length of at least 100 ribonucleotides and (e) a second post-circularization element; and wherein the first post-circularization element and the second post-circularization element together form a circularization junction.

In some embodiments, the first spacer element has a length of between 100 to 500 (e.g., 100 to 450, 100 to 400, 100 to 350, 100 to 300, 100 to 250, 100 to 200, 100 to 150, 110 to 500, 110 to 450, 110 to 400, 110 to 350, 110 to 300, 110 to 250, 110 to 200, 110 to 150, 120 to 500, 150 to 500, 200 to 500, 250 to 500, 300 to 500, 350 to 500, 400 to 500, and 450 to 500) ribonucleotides. In some embodiments, the first spacer element has a length of between 100 and 300 (e.g., 100 to 250, 100 to 200, 100 to 150, 110 to 300, 120 to 300, 150 to 300, 200 to 300, and 250 to 300). In some embodiments, the second spacer element has a length of between 100 to 500 (e.g., 100 to 450, 100 to 400, 100 to 350, 100 to 300, 100 to 250, 100 to 200, 100 to 150, 110 to 500, 120 to 500, 150 to 500, 200 to 500, 250 to 500, 300 to 500, 350 to 500, 400 to 500, and 450 to 500) ribonucleotides. In some embodiments, the first spacer element and the second spacer element each comprise 110 to 500 (e.g., 110 to 450, 110 to 400, 110 to 350, 110 to 300, 110 to 250, 110 to 200, 110 to 150, 150 to 500, 200 to 500, 250 to 500, 300 to 500, 350 to 500, 400 to 500, and 450 to 500) ribonucleotides. In some embodiments, the first spacer element and the second spacer element each comprise 120 to 500 (e.g., 120 to 450, 120 to 400, 120 to 350, 120 to 300, 120 to 250, 120 to 200, 120 to 150, 150 to 500, 200 to 500, 250 to 500, 300 to 500, 350 to 500, 400 to 500, and 450 to 500) ribonucleotides. In some embodiments, the first spacer element and the second spacer element each have a length of 150 to 500 (e.g., 150 to 450, 150 to 400, 150 to 350, 150 and 300, 150 and 250, 150 and 200, 200 500, 250 and 500, 300 and 500, 350 and 500, 400 and 500, or 450 and 500) ribonucleotides. In some embodiments, the first spacer element and the second spacer element each have a length of 200 to 500 (e.g., 200 to 450, 200 to 400, 200 to 350, 200 to 300, 200 to 250, 250 to 500, 300 to 500, 350 to 500, 400 to 500, or 450 to 500) ribonucleotides. In some embodiments, the first spacer element and the second spacer element each have a length of 100 to 300 (e.g., 100 to 280, 100 to 260, 100 to 240, 100 to 220, 100 to 200, 100 to 180, 100 to 160, 100 to 140, 100 to 120, 120 to 300, 140 to 300, 160 to 300, 180 to 300, 200 to 300, 220 to 300, 240 to 300, 260 to 300, and 280 to 300) ribonucleotides. In some embodiments, the first spacer element and the second spacer element each have a length of 100 to 200 (e.g., 100 to 190, 100 to 180, 100 to 170, 100 to 160, 100 to 150, 100 to 140, 100 to 130, 100 to 120, 100 to 110, 110 to 200, 120 to 200, 130 to 200, 140 to 200, 150 to 200, 160 to 200, 170 to 200, 180 to 200, and 90 to 200) ribonucleotides. In some embodiments, the first spacer element and the second spacer element each have a length of 110 to 300 (e.g., 110 to 280, 110 to 260, 110 to 240, 110 to 220, 110 to 200, 110 to 180, 110 to 160, 110 to 140, 140 to 300, 160 to 300, 180 to 300, 200 to 300, 220 to 300, 240 to 300, 260 to 300, and 280 to 300) ribonucleotides. In some embodiments, the first spacer element and the second spacer element each have a length of 120 to 300 (e.g., 120 to 280, 120 to 260, 120 to 240, 120 to 220, 120 to 200, 120 to 180, 120 to 160, 120 to 140, 140 to 300, 160 to 300, 180 to 300, 200 to 300, 220 to 300, 240 to 300, 260 to 300, and 280 to 300) ribonucleotides. In some embodiments, the first spacer element and the second spacer element each have a length of 150 to 300 (e.g., 150 to 290, 150 to 280, 150 to 270, 150 to 260, 150 to 250, 150 to 240, 150 to 230, 150 to 220, 150 to 210, 150 to 200, 150 to 190, 150 to 180, 150 to 170, 150 to 160, 160 to 300, 170 to 300, 180 to 300, 190 to 300, 200 to 300, 210 to 300, 220 to 300, 230 to 300, 240 to 300, 250 to 300, 260 to 300, 270 to 300, 280 to 300, and 290 to 300) ribonucleotides. In some embodiments, the difference between the length of the first spacer element and the length of the second spacer element is 0 to 100 (e.g., 0 and 10, 0 and 20, 0 and 30, 0, and 40, 0 and 50, 0 to 60, 0 to 70, 0 to 80, 0 to 90, 10 to 100, 20 to 100, 30 to 100, 40 to 100, 50 to 100, 60 to 100, 70 to 100, 80 to 100, 90 to 100, 5 to 10, 5 to 20, 10 to 30 or 5 to 50) nucleotides. In some embodiments, the difference between the length of the first spacer element and the length of the second spacer element is 0 to 50 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50) nucleotides. In some embodiments, the first spacer element and the second spacer element are the same number of ribonucleotides in length.

In some embodiments, the first spacer or the second spacer element consists of: (i) a polyA region comprising 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, and 100%) adenosine residues; (ii) a polyAC region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, and 100%) adenosine or cytosine residues; (iii) a polyAU region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, and 100%) adenosine or uridine residues; or (iv) a polyAG region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, and 100%) adenosine or guanosine residues.

In some embodiments, the polyribonucleotide exhibits at least 2-fold greater stability in comparison to a polyribonucleotide without the first spacer element or the second spacer element. In some embodiments, the polyribonucleotide may be detected for at least 2-fold longer in comparison to a polyribonucleotide without the first spacer element or the second spacer element after the polyribonucleotide is administered to a subject. In some embodiments, the polynucleotide cargo encodes a polypeptide, wherein the polypeptide has at least 4-fold greater expression in comparison to the polyribonucleotide without the first spacer element or the second spacer element.

In some embodiments, the polyribonucleotide cargo comprises an expression sequence. In some embodiments, the polyribonucleotide cargo comprises an IRES operably linked an expression sequence. In some embodiments, the expression sequence further comprises a 3′ untranslated region or a 5′ untranslated region. In some embodiments, the expression sequence encodes a polypeptide.

In some embodiments, the polyribonucleotide comprises between 500 and 20,000 (e.g., between 500 to 15,000, 500 to 10,000, 500 to 5,000, 500 to 1,000, 1,000 to 20,000, 5,000 to 20,000, 10,000 to 20,000, and 15,000 to 20,000) ribonucleotides. In some embodiments, the polyribonucleotide comprises between 2,000 and 20,000 (e.g., 2,000 to 5,000, 2,000 to 7,500, 2,000 to 10,000, 2,000 to 12,500, 2,000 to 15,000, 2,000 to 17,500, 5,000 to 20,000, 7,500 to 20,000, 10,000 to 20,000, 12,500 to 20,000, 15,000 to 20,000, and 17,500 to 20,000) ribonucleotides.

In some embodiments, the circularization junction comprises a splice junction. In some embodiments, the first post-circularization element comprises a first exon fragment and the second post-circularization element comprises a second exon fragment, and wherein the first exon fragment and the second exon fragment are joined by the splice junction. In some embodiments, the circularization junction comprises an oligonucleotide splint that is hybridized to the first post-circularization element and to the second post-circularization element. In some embodiments, the oligonucleotide splint is a DNA splint or an RNA splint. In some embodiments, the first post-circularization element comprises a region that is capable of hybridizing to a first region of the oligonucleotide splint and the second post-circularization element comprises a region that is capable of hybridizing to a second region of the oligonucleotide splint. In some embodiments, the first circularization element comprises a first catalytic intron fragment, a first splice site dinucleotide, and a first exon fragment; and the second circularization element comprises a second catalytic intron fragment, a second splice site dinucleotide, and a second exon fragment. In some embodiments, the first catalytic intron fragment and the second catalytic intron fragment, together, are capable of self-splicing thereby covalently joining the first exon region and the second exon region to produce a circular polyribonucleotide. In some embodiments, the first catalytic intron fragment and the second catalytic intron fragment are from a cyanobacterium Anabaena pre-tRNA-Leu gene or a Tetrahymena pre-rRNA. In some embodiments, the first circularization element comprises a region that is capable of hybridizing to a first region of an oligonucleotide splint and the second circularization element comprises a region that is capable of hybridizing to a second region of the oligonucleotide splint. In another aspect the disclosure provides a DNA vector comprising an RNA polymerase promoter operably linked to a sequence that encodes the any one of the linear polyribonucleotides described herein.

In another aspect the disclosure provides a circular polyribonucleotide produced from any one of the linear polyribonucleotides or from the DNA vector described herein. In another aspect the disclosure provides a pharmaceutical composition comprising any one of the linear polyribonucleotides, circular polyribonucleotides, or the DNA vector described herein and a pharmaceutically acceptable excipient.

In another aspect the disclosure provides a method of expressing a polypeptide in a cell or a subject, the method comprising providing to the cell or the subject any one of the linear polyribonucleotides, the circular polyribonucleotides, the DNA vector, or the pharmaceutical composition described herein.

In another aspect the disclosure provides a method of producing a circular polyribonucleotide from any one of the linear polyribonucleotides described herein, the method comprising providing the linear polyribonucleotide under conditions suitable for self-splicing of the linear polyribonucleotide to produce a circular polyribonucleotide.

### Definitions

To facilitate the understanding of this disclosure, a number of terms are defined below. Terms defined herein have meanings as commonly understood by a person of ordinary skill in the areas relevant to the disclosure. Terms such as “a”, “an,” and “the” are not intended to refer to only a singular entity but include the general class of which a specific example may be used for illustration. The term “or” is used to mean “and/or” unless explicitly indicated to refer to alternatives only or the alternative are mutually exclusive, although the disclosure supports a definition that refers to only alternatives and “and/or.” The terminology herein is used to describe specific embodiments, but their usage is not to be taken as limiting, except as outlined in the claims.

As used herein, any values provided in a range of values include both the upper and lower bounds, and any values contained within the upper and lower bounds.

As used herein, the term “about” refers to a value that is within ±10% of a recited value.

As used herein, the term “between” refers to all values that are greater than or equal to the initial value and less than or equal to the endpoint, such that the range of values between two values includes the endpoints of the range. For example, between 1 and 5 refers to all values ≥1 and ≤5 such that the endpoints of 1 and 5 are included in the contemplated range.

As used herein, the term “carrier” is a compound, composition, reagent, or molecule that facilitates the transport or delivery of a composition (e.g., a circular polyribonucleotide) into a cell by a covalent modification of the circular polyribonucleotide, via a partially or completely encapsulating agent, or a combination thereof. Non-limiting examples of carriers include carbohydrate carriers (e.g., an anhydride-modified phytoglycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

As used herein, the terms “circRNA,” “circular polyribonucleotide,” “circular RNA,” and “circular polyribonucleotide molecule” are used interchangeably and mean a polyribonucleotide molecule that has a structure having no free ends (i.e., no free 3′ and/or 5′ ends), for example a polyribonucleotide molecule that forms a circular or end-less structure through covalent (e.g., covalently closed) or non-covalent bonds. The circular polyribonucleotide may be e.g., a covalently closed polyribonucleotide.

As used herein, the term “circularization efficiency” is a measurement of resultant circular polyribonucleotide versus its non-circular starting material.

As used herein, the terms “disease,” “disorder,” and “condition” each refer to a state of sub-optimal health, for example, a state that is or would typically be diagnosed or treated by a medical professional.

As used herein, the term “expression augmenting element” refers to a component of a polyribonucleotide construct which increases the expression of a polypeptide cargo encoded by the polyribonucleotide in comparison to a polyribonucleotide construct lacking the expression element. For example, in embodiments, the expression augmenting element is a translation enhancer or a stability element. In embodiments, the expression augmenting element increases expression of the polypeptide cargo by increasing the length of time circRNA persists before it is degraded, or it increases the concentration of the polypeptide cargo that is expressed over a fixed time period.

As used herein, the term “expression sequence” is a nucleic acid sequence that encodes a product, e.g., a polypeptide. An exemplary expression sequence that codes for a polypeptide may include a plurality of nucleotide triads, each of which can code for an amino acid and is termed as a “codon.”

As used herein, the term “fragment,” with respect to a polypeptide or a nucleic acid sequence, refers to a continuous, less than a whole portion of a sequence of the polypeptide or the nucleic acid. A fragment of a polypeptide for instance, refers to continuous, less than a whole fraction (e.g., at least 20%, 30%, 40%, 50%, 60%, 70%, 80%, 90%, 95%, or 99% of the entire length) of the sequence such as a sequence disclosed herein. It is understood that all the present disclosure contemplates fragments of all polypeptides disclosed herein.

As used herein, the term “fusion” refers a single, contiguous molecule containing two or more different elements. For example, the fusion elements described herein may include a translation enhancer fused (e.g., joined or connected) to a stability element, a translation enhancer fused (e.g., joined or connected) to a spacer element, or a stability element fused (e.g., joined or connected) to a spacer element. Fusion elements may have functional properties derived from each of the original elements. In some embodiments, a translation enhancer is connected at its 5′ end to a spacer element. In some embodiments, a translation enhancer is connected at its 3′ end to a spacer element. In some embodiments, a stability element is connected at its 5′ end to a spacer element. In some embodiments, a stability element is connected at its 3′ end to a spacer element. In some embodiments, a translation enhancer is connected at its 5′ end to a stability element. In some embodiments, a translation enhancer is connected at its 3′ end to a translation stability.

As used herein, the term “GC content” refers to the percentage of guanine (G) and cytosine (C) in a nucleic acid sequence. The formula for calculation of the GC content is (G+C)/(A+G+C+U)×100% (for RNA) or (G+C)/(A+G+C+T)×100% (for DNA). Likewise, the term “uridine content” refers to the percentage of uridine (U) in a nucleic acid sequence. The formula for calculation of the uridine content is U/(A+G+C+U)×100%. Likewise, the term “thymidine content” refers to the percentage of thymidine (T) in a nucleic acid sequence. The formula for calculation of the thymidine content is T/(A+G+C+T)×100%.

By “heterologous” is meant to occur in a context other than in the naturally occurring (native) context. A “heterologous” polynucleotide sequence indicates that the polynucleotide sequence is being used in a way other than what is found in that sequence's native genome. For example, a “heterologous promoter” is used to drive transcription of a sequence that is not one that is natively transcribed by that promoter; thus, a “heterologous promoter” sequence is often included in an expression construct by means of recombinant nucleic acid techniques. The term “heterologous” is also used to refer to a given sequence that is placed in a non-naturally occurring relationship to another sequence; for example, a heterologous coding or non-coding nucleotide sequence is commonly inserted into a genome by genomic transformation techniques, resulting in a genetically modified or recombinant genome.

As used herein, the term “impurity” is an undesired substance present in a composition, e.g., a pharmaceutical composition as described herein. In some embodiments, an impurity is a process-related impurity. In some embodiments, an impurity is a product-related substance other than the desired product in the final composition, e.g., other than the active drug ingredient, e.g., circular polyribonucleotide, as described herein. As used herein, the term “process-related impurity” is a substance used, present, or generated in the manufacturing of a composition, preparation, or product that is undesired in the final composition, preparation, or product other than the linear polyribonucleotides described herein. In some embodiments, the process-related impurity is an enzyme used in the synthesis or circularization of polyribonucleotides. As used herein, the term “product-related substance” is a substance or byproduct produced during the synthesis of a composition, preparation, or product, or any intermediate thereof. In some embodiments, the product-related substance is deoxyribonucleotide fragments. In some embodiments, the product-related substance is deoxyribonucleotide monomers. In some embodiments, the product-related substance is one or more of: derivatives or fragments of polyribonucleotides described herein, e.g., fragments of 10, 9, 8, 7, 6, 5, or 4 ribonucleic acids, monoribonucleic acids, diribonucleic acids, or triribonucleic acids.

As used herein, “increasing fitness” or “promoting fitness” of a subject refer to any favorable alteration in physiology, or of any activity carried out by a subject organism, as a consequence of administration of a peptide or polypeptide described herein, including, but not limited to, any one or more of the following desired effects: (1) increased tolerance of biotic or abiotic stress by about 10%, 20%, 30%, 40%, 50%, 60%, 70%, 80%, 90%, 95%, 99%, 100% or more; (2) increased yield or biomass by about 10%, 20%, 30%, 40%, 50%, 60%, 70%, 80%, 90%, 95%, 99%, 100% or more; (3) modified flowering time by about 10%, 20%, 30%, 40%, 50%, 60%, 70%, 80%, 90%, 95%, 99%, 100% or more; (4) increased resistance to pests or pathogens by about 10%, 20%, 30%, 40%, 50%, 60%, 70%, 80%, 90%, 95%, 99%, 100% or more, (4) increased resistance to herbicides by about 10%, 20%, 30%, 40%, 50%, 60%, 70%, 80%, 90%, 95%, 99%, 100% or more; (5) increasing a population of a subject organism (e.g., an agriculturally important insect) by about 10%, 20%, 30%, 40%, 50%, 60%, 70%, 80%, 90%, 95%, 99%, 100% or more; (6) increasing the reproductive rate of a subject organism (e.g., insect, e.g., bee or silkworm) by about 10%, 20%, 30%, 40%, 50%, 60%, 70%, 80%, 90%, 95%, 99%, 100% or more; (7) increasing the mobility of a subject organism (e.g., insect, e.g., bee or silkworm) by about 10%, 20%, 30%, 40%, 50%, 60%, 70%, 80%, 90%, 95%, 99%, 100% or more; (8) increasing the body weight of a subject organism (e.g., insect, e.g., bee or silkworm) by about 10%, 20%, 30%, 40%, 50%, 60%, 70%, 80%, 90%, 95%, 99%, 100% or more; (9) increasing the metabolic rate or activity of a subject organism (e.g., insect, e.g., bee or silkworm) by about 10%, 20%, 30%, 40%, 50%, 60%, 70%, 80%, 90%, 95%, 99%, 100% or more; (10) increasing pollination (e.g., number of plants pollinated in a given amount of time) by a subject organism (e.g., insect, e.g., bee or silkworm) by about 10%, 20%, 30%, 40%, 50%, 60%, 70%, 80%, 90%, 95%, 99%, 100% or more; (11) increasing production of subject organism (e.g., insect, e.g., bee or silkworm) byproducts (e.g., honey from a honeybee or silk from a silkworm) by about 10%, 20%, 30%, 40%, 50%, 60%, 70%, 80%, 90%, 95%, 99%, 100% or more; (12) increasing nutrient content of the subject organism (e.g., insect) (e.g., protein, fatty acids, or amino acids) by about 10%, 20%, 30%, 40%, 50%, 60%, 70%, 80%, 90%, 95%, 99%, 100% or more; or (13) increasing a subject organism's resistance to pesticides (e.g., a neonicotinoid (e.g., imidacloprid) or an organophosphorus insecticide (e.g., a phosphorothioate, e.g., fenitrothion)) by about 10%, 20%, 30%, 40%, 50%, 60%, 70%, 80%, 90%, 95%, 99%, 100% or more, (14) increasing health or reducing disease of a subject organism such as a human or non-human animal. An increase in host fitness can be determined in comparison to a subject organism to which the polyribonucleotide has not been administered. Conversely, “decreasing fitness” of a subject refers to any unfavorable alteration in physiology, or of any activity carried out by a subject organism, as a consequence of administration of a peptide or polypeptide described herein, including, but not limited to, any one or more of the following intended effects: (1) decreased tolerance of biotic or abiotic stress by about 10%, 20%, 30%, 40%, 50%, 60%, 70%, 80%, 90%, 95%, 99%, 100% or more; (2) decreased yield or biomass by about 10%, 20%, 30%, 40%, 50%, 60%, 70%, 80%, 90%, 95%, 99%, 100% or more; (3) modified flowering time by about 10%, 20%, 30%, 40%, 50%, 60%, 70%, 80%, 90%, 95%, 99%, 100% or more; (4) decreased resistance to pests or pathogens by about 10%, 20%, 30%, 40%, 50%, 60%, 70%, 80%, 90%, 95%, 99%, 100% or more, (4) decreased resistance to herbicides by about 10%, 20%, 30%, 40%, 50%, 60%, 70%, 80%, 90%, 95%, 99%, 100% or more; (5) decreasing a population of a subject organism (e.g., an agriculturally important insect) by about 10%, 20%, 30%, 40%, 50%, 60%, 70%, 80%, 90%, 95%, 99%, 100% or more; (6) decreasing the reproductive rate of a subject organism (e.g., insect, e.g., bee or silkworm) by about 10%, 20%, 30%, 40%, 50%, 60%, 70%, 80%, 90%, 95%, 99%, 100% or more; (7) decreasing the mobility of a subject organism (e.g., insect, e.g., bee or silkworm) by about 10%, 20%, 30%, 40%, 50%, 60%, 70%, 80%, 90%, 95%, 99%, 100% or more; (8) decreasing the body weight of a subject organism (e.g., insect, e.g., bee or silkworm) by about 10%, 20%, 30%, 40%, 50%, 60%, 70%, 80%, 90%, 95%, 99%, 100% or more; (9) decreasing the metabolic rate or activity of a subject organism (e.g., insect, e.g., bee or silkworm) by about 10%, 20%, 30%, 40%, 50%, 60%, 70%, 80%, 90%, 95%, 99%, 100% or more; (10) decreasing pollination (e.g., number of plants pollinated in a given amount of time) by a subject organism (e.g., insect, e.g., bee or silkworm) by about 10%, 20%, 30%, 40%, 50%, 60%, 70%, 80%, 90%, 95%, 99%, 100% or more; (11) decreasing production of subject organism (e.g., insect, e.g., bee or silkworm) byproducts (e.g., honey from a honeybee or silk from a silkworm) by about 10%, 20%, 30%, 40%, 50%, 60%, 70%, 80%, 90%, 95%, 99%, 100% or more; (12) decreasing nutrient content of the subject organism (e.g., insect) (e.g., protein, fatty acids, or amino acids) by about 10%, 20%, 30%, 40%, 50%, 60%, 70%, 80%, 90%, 95%, 99%, 100% or more; or (13) decreasing a subject organism's resistance to pesticides (e.g., a neonicotinoid (e.g., imidacloprid) or an organophosphorus insecticide (e.g., a phosphorothioate, e.g., fenitrothion)) by about 10%, 20%, 30%, 40%, 50%, 60%, 70%, 80%, 90%, 95%, 99%, 100% or more, (14) decreasing health or reducing disease of a subject organism such as a human or non-human animal. A decrease in host fitness can be determined in comparison to a subject organism to which the polyribonucleotide has not been administered. It will be apparent to one of skill in the art that certain changes in the physiology, phenotype, or activity of a subject, e.g., modification of flowering time in a plant, can be considered to increase fitness of the subject or to decrease fitness of the subject, depending on the context (e.g., to adapt to a change in climate or other environmental conditions). For example, a delay in flowering time (e.g., about 10%, 20%, 30%, 40%, 50%, 60%, 70%, 80%, 90%, 95%, 99%, 100% fewer plants in a population flowering at a given calendar date) can be a beneficial adaptation to later or cooler springtimes and thus be considered to increase a plant's fitness; conversely, the same delay in flowering time in the context of earlier or warmer springtimes can be considered to decrease a plant's fitness.

As used herein, the terms “linear RNA,” “linear polyribonucleotide,” and “linear polyribonucleotide molecule” are used interchangeably and mean a monoribonucleotide molecule or polyribonucleotide molecule having a 5′ and 3′ end. One or both of the 5′ and 3′ ends may be free ends or joined to another moiety. In some embodiments, the linear RNA has a 5′ end or 3′ end that is modified or protected from degradation (e.g., by a 5′ end protectant or a 3′ end protectant). In some embodiments, the linear RNA has non-covalently linked 5′ or 3′ ends. Linear RNA includes RNA that has not undergone circularization (e.g., is pre-circularized) and can be used as a starting material for circularization.

As used herein, the term “linear counterpart” is a polyribonucleotide molecule (and its fragments) having the same or similar nucleotide sequence (e.g., 100%, 95%, 90%, 85%, 80%, 75%, or any percentage therebetween sequence similarity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence similarity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween sequence similarity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further comprises a 5′ cap. In some embodiments, the linear counterpart further comprises a poly adenosine tail. In some embodiments, the linear counterpart further comprises a 3′ UTR. In some embodiments, the linear counterpart further comprises a 5′ UTR.

As used herein, the term “modified ribonucleotide” is a nucleotide with at least one modification to the sugar, the nucleobase, or the internucleoside linkage.

The term “pharmaceutical composition” is intended to also disclose that the circular polyribonucleotide included within a pharmaceutical composition can be used for the treatment of the human or animal body (e.g., veterinary use) by therapy. It is thus meant to be equivalent to “a circular polyribonucleotide for use in therapy”.

The term “polyA based spacer element” or “polyA spacer element” refers to a spacer element comprising an untranslated, contiguous region of nucleic acid molecules of at least 4 nucleotides in length and consisting of one or more individual adenine (A) residues in combination with one or more (A), thymine (T), cytosine (C), guanine (G), or uracil (U) residues. For example, in some embodiments, the polyA based spacer element is a polyA region, which may be sequence of adenine residues. In other embodiments, the polyA based spacer element is a polyAT region, which is a combination of adenine and thymine residues. In other embodiments, the polyA based spacer element is a poly AU region, which may be a combination of adenine and uracil residues. In some embodiments, the polyA based spacer element is a polyAG region, which is a combination of adenine and guanine residues. A polyA based spacer element comprises between 50% to 100% (e.g., between 50% to 90%, 50% to 80%, 50% to 70%, 50% to 60%, 60% to 100%, 70% to 100%, 80% to 100%, 90% to 100%, 70% to 90%, or 60% to 80%) adenine residues.

The term “polynucleotide” as used herein means a molecule including one or more nucleic acid subunits, or nucleotides, and can be used interchangeably with “nucleic acid” or “oligonucleotide.” A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO3) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose. In some examples, a polynucleotide is deoxyribonucleic acid (DNA), ribonucleic acid (RNA), or derivatives or variants thereof. In some cases, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular. A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. As embodied herein, the polynucleotide sequences may include isolated and purified DNA/RNA molecules, synthetic DNA/RNA molecules, and synthetic DNA/RNA analogs.

“Polydeoxyribonucleotides,” “deoxyribonucleic acids,” and “DNA” mean macromolecules that include multiple deoxyribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, which include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof).

Polynucleotides, e.g., polyribonucleotides or polydeoxyribonucleotides, may include one or more nucleotide variants, including nonstandard nucleotide(s), non-natural nucleotide(s), nucleotide analog(s) and/or modified nucleotides. Examples of modified nucleotides include, but are not limited to diaminopurine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5 -methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, wybutoxosine, pseudo uracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, 3-(3-amino-3-carboxypropyl) uridine 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxy succinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A. NAT. CHEM. BIOL. 2012 July; 8(7):612-4, which is herein incorporated by reference for all purposes.

As used herein, “polypeptide” means a polymer of amino acid residues (natural or unnatural) linked together most often by peptide bonds. The term, as used herein, refers to proteins, polypeptides, and peptides of any size, structure, or function. Polypeptides can include gene products, naturally occurring polypeptides, synthetic polypeptides, homologs, orthologs, paralogs, fragments and other equivalents, variants, and analogs of the foregoing. A polypeptide can be a single molecule or a multi-molecular complex such as a dimer, trimer, or tetramer. They can also comprise single chain or multichain polypeptides such as antibodies or insulin and can be associated or linked. Most commonly disulfide linkages are found in multichain polypeptides. The term polypeptide can also apply to amino acid polymers in which one or more amino acid residues are an artificial chemical analogue of a corresponding naturally occurring amino acid.

As used herein, the term “polyribonucleotide cargo” herein includes any sequence including at least one polyribonucleotide. In embodiments, the polyribonucleotide cargo includes one or multiple expression sequences, wherein each expression sequence encodes a polypeptide. In embodiments, the polyribonucleotide cargo includes one or multiple noncoding sequences, such as a polyribonucleotide having regulatory or catalytic functions. In embodiments, the polyribonucleotide cargo includes a combination of expression and noncoding sequences. In embodiments, the polyribonucleotide cargo includes one or more polyribonucleotide sequence described herein, such as one or multiple regulatory elements, internal ribosomal entry site (IRES) elements, or spacer elements.

As used herein, the term “prevent” means to reduce the likelihood of developing a disease, disorder, or condition, or alternatively, to reduce the severity or frequency of symptoms in a subsequently developed disease or disorder. The polyribonucleotides described herein can be administered to a subject who is at increased risk of developing a disease or disorder relative to a member of the general population in order to prevent the development of, or lessen the severity of, the disease or condition. The polyribonucleotides described herein can be administered as a prophylactic (e.g., before development of any symptom or manifestation of a disease or disorder).

As used herein, the terms “purify,” “purifying,” and “purification” refer to one or more steps or processes of removing impurities (e.g., a process-related impurity (e.g., an enzyme), a process-related substance (e.g., a deoxyribonucleotide fragment, a deoxyribonucleotide monomer)) or by-products (e.g., linear RNA) from a sample containing a mixture circular RNA and linear RNA, among other substances, to produce a composition containing an enriched population of circular RNA with a reduced level of an impurity (e.g., a process-related impurity (e.g., an enzyme), a process-related substance (e.g., deoxyribonucleotide fragment, deoxyribonucleotide monomer)) or by-product (e.g., linear RNA) as compared to the original mixture or in which the linear RNA or substances have been reduced by 40% or more by mass (e.g., 50%, 60%, 70%, 80%, 85%, 90%, 95%, 97%, or 99% or more) relative to a starting mixture.

As used herein, the terms “pure” and “purity” refer to the extent to which an analyte (e.g., circular RNA) has been isolated and is free of other components. In the context of nucleic acids (e.g., polyribonucleotides), purity of an isolated nucleic acid (e.g., circular RNA) can be expressed with regard to the population of nucleic acids that is free of any contaminants, impurities, or by-products (e.g., linear RNA and other substances). For example, purity of a population of circular RNA indicates how much of the population is circular RNA by total mass of the isolated material, which may be determined using, e.g., pure circular RNA as a reference. A level of purity found in the disclosure can be 5%, 10%, 15%, 20%, 25%, 30%, 35%, 40%, 45%, 50%, 55%, 60%, 65%, 70%, 75%, 80%, 85%, 90%, 95%, greater than 95%, or greater than 99% (w/w). In some embodiments, the level of contaminants or impurities or by-products is no more than about 20%, 15%, 12%, 10%, 9%, 8%, 7%, 6%, 5%, 4%, 3%, 2%, or 1% (w/w). Purity can be determined by detecting a level of a specific analyte (e.g., circular RNA) or a specific impurity or by-product (e.g., linear RNA) using gel electrophoresis, spectrophotometry (e.g., NanoDrop by ThermoFisher Scientific), or other technique suitable for measuring purity of a population of nucleic acids and calculating a percentage of the analyte (w/w) relative to the total nucleic acid content (e.g., as determined by an assay known in the art).

As used herein, the phrase “substantially free of one or more impurities or by-products” refers to a property of a sample, such as a sample containing an enriched population of circular RNA, that is free of one or more impurities or by-products (e.g., one or more impurities or by-products disclosed herein) or contains a minimal amount of the one or more impurities or by-products. A minimal amount of the one or more impurities or by-products may be no more than 20% (w/w) (e.g., no more than 19%, 18%, 17%, 16%, 15%, 14%, 13%, 12%, 11%, 10%, 9%, 8%, 7%, 6%, 5%, 4%, 3%, 2%, 1% (w/w), or less). In another example, the sample or the enriched population of circular RNA is substantially free of one or more impurities or by-products if the one or more impurities or by-products are present in an amount that is less than 15% (w/w) (e.g., no more than 14%, 13%, 12%, 11%, 10%, 9%, 8%, 7%, 6%, 5%, 4%, 3%, 2%, 1% (w/w), or less). In another example, the sample or the enriched population of circular RNA is substantially free of one or more impurities or by-products if the one or more impurities or by-products are present in an amount that is less than 10% (w/w) (e.g., no more than 9%, 8%, 7%, 6%, 5%, 4%, 3%, 2%, 1% (w/w), or less). In another example, the sample or the enriched population of circular RNA is substantially free of one or more impurities or by-products if the one or more impurities or by-products are present in an amount that is less than 5% (w/w) (e.g., no more than 4%, 3%, 2%, 1% (w/w) or less). In yet another example, the sample or the enriched population of circular RNA is substantially free of one or more impurities or by-products if the one or more impurities or by-products are present in an amount that is less than 1% (no more than 0.9%, 0.8%, 0.7%, 0.6%, 0.5%, 0.4%, 0.3%, 0.2%, 0.1% (w/w), or less).

As used herein, the term “regulatory element” is a moiety, such as a nucleic acid sequence, that modifies expression of an expression sequence within the circular polyribonucleotide.

As used herein, the term “replication element” is a sequence and/or motif useful for replication or that initiates transcription of the circular polyribonucleotide.

As used herein, the term “RNA equivalent” refers to an RNA sequence that is the RNA equivalent of a DNA sequence. An RNA equivalent of a DNA sequence therefore refers to a DNA sequence in which each of the thymidine (T) residues is replaced by a uridine (U) residue. The disclosure specifically contemplates that any of these DNA sequences may be converted to the corresponding RNA sequence and included in an RNA molecule described herein.

As used herein, the term “sequence identity” is determined by alignment of two peptide or two nucleotide sequences using a global or local alignment algorithm. Sequences may then be referred to as “substantially identical” or “essentially similar” when they (when optimally aligned by for example the programs GAP or BESTFIT using default parameters) share at least a certain minimal percentage of sequence identity. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is a nwsgapdna.cmp scoring matrix and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or EmbossWin version 2.10.0 (using the program “needle”). Alternatively, or additionally, percent identity may be determined by searching against databases, using algorithms such as FASTA, BLAST, etc. Sequence identity refers to the sequence identity over the entire length of the sequence.

A “signal sequence” refers to a polypeptide sequence, e.g., between 10 and 45 amino acids in length, that is present at the N-terminus of a polypeptide sequence of a nascent protein which targets the polypeptide sequence to the secretory pathway.

As used herein, the term “spacer element” refers to any contiguous nucleotide sequence (e.g., of one or more nucleotides) that provides distance or flexibility between two adjacent polynucleotide regions. Spacer elements may be present in between any of the nucleic acid elements described herein. Spacer element may also be present within a nucleic acid element described herein.

As used herein, the term “stability element” refers to a polyribonucleotide element which increases the stability of the polyribonucleotide in comparison to a polyribonucleotide lacking the stability element. The stability element may increase stability of the polyribonucleotide such that the polyribonucleotide degrades more slowly.

As used herein, the term “subject” refers to an organism, such as an animal, plant, or microbe. In embodiments, the subject is a vertebrate animal (e.g., mammal, bird, fish, reptile, or amphibian). In embodiments, the subject is a human. In embodiments, the subject is a non-human mammal. In embodiments, the subject is a non-human mammal such as a non-human primate (e.g., monkeys, apes), ungulate (e.g., cattle, buffalo, bison, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusc. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a geophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

As used herein, the term “translation enhancer” refers to a polyribonucleotide element, which allows for translation of mRNA via recruitment of a ribosome, translation initiation factor directly or through interacting with RNA-binding protein. The polyribonucleotides including a translation enhancer may, for example, demonstrate increased stability and/or increased expression of a polyribonucleotide cargo in comparison to a polyribonucleotide lacking the translation enhancer.

As used herein, the term “termination element” is a moiety, such as a nucleic acid sequence, that terminates translation of the expression sequence in the circular polyribonucleotide.

As used herein, the term “total ribonucleotide molecules” means the total amount of any ribonucleotide molecules, including linear polyribonucleotide molecules, circular polyribonucleotide molecules, monomeric ribonucleotides, other polyribonucleotide molecules, fragments thereof, and modified variations thereof, as measured by total mass of the ribonucleotide molecules.

As used herein, the term “translation efficiency” is a rate or amount of protein or peptide production from a ribonucleotide transcript. In some embodiments, translation efficiency can be expressed as amount of protein or peptide produced per given amount of transcript that codes for the protein or peptide, e.g., in a given period of time, e.g., in a given translation system, e.g., an in vitro translation system like rabbit reticulocyte lysate, or an in vivo translation system like a eukaryotic cell or a prokaryotic cell.

As used herein, the term “translation initiation sequence” is a nucleic acid sequence that initiates translation of an expression sequence in the circular polyribonucleotide.

As used herein, the terms “treat” and “treating” refer to a prophylactic or therapeutic treatment of a disease or condition, in a subject. The effect of treatment can include reversing, alleviating, reducing severity of, curing, inhibiting the progression of, reducing the likelihood of recurrence of the disease or condition or one or more symptoms or manifestations of the disease or condition, stabilizing (i.e., not worsening) the state of the disease or condition, or preventing the spread of the disease or condition as compared to the state or the condition of the disease or condition in the absence of the therapeutic treatment.

As used herein, a “variant” refers to a polypeptide which includes at least one alteration, e.g., a substitution, insertion, deletion, and/or fusion, at one or more residue positions, as compared to the parent or wild-type polypeptide. A variant may include between 1 and 10, 10 and 20, 20 and 50, 50 and 100, or more alterations.

### BRIEF DESCRIPTION OF THE DRAWINGS

FIGS. 1A-1F are schematic diagrams of exemplary linear and circular polyribonucleotide constructs having spacer element(s) and/or expression augmenting element(s).

FIG. 2 is a table showing the circularization efficiency (% circRNA) of single spacer designs or dual spacer design constructs having a spacer element(s) between 50 and 300 nucleotides in length, wherein the construct encodes a Gluc polypeptide or human erythropoietin.

FIG. 3A is a table of translation enhancers, including motifs and sequences.

FIG. 3B is a table showing the circularization efficiency (% circRNA) of single design and dual design constructs having a spacer element and/or a translation enhancer and encoding a Gluc polypeptide.

FIG. 4A is a bar graph showing expression of Gluc in Hela cells transfected with circular RNAs having a spacer element and encoding a Gluc polypeptide, 24 and 48 hours after transfection.

FIG. 4B is a graph showing expression of EPO in Hela cells transfected with circular RNAs having a spacer element and encoding a human erythropoietin, 24 and 48 hours after transfection.

FIG. 5 is a bar graph showing expression of Gluc in Hela cells transfected with circular RNAs having a single spacer design or a dual spacer design and encoding a Gluc polypeptide, 24 hours after transfection.

FIG. 6A is a bar graph showing expression of Gluc in Hela cells transfected with circular RNAs having a dual spacer design and encoding a Gluc polypeptide, 4, 24, and 48 hours after transfection.

FIG. 6B is a bar graph showing expression of EPO in Hela cells transfected with circular RNAs having a dual spacer design and encoding a human erythropoietin, 4, 24, and 48 hours after transfection.

FIG. 7A is a bar graph showing expression of Gluc in A549 cells transfected with circular RNAs having a spacer element and a translation enhancer and encoding a Gluc polypeptide.

FIG. 7B is a bar graph showing expression of EPO in A549 cells transfected with circular RNAs having a spacer element and a translation enhancer and encoding human erythropoietin, 24 hours after transfection.

FIG. 8 is a bar graph showing the expression of EPO in HEK cells transfected with a circular polyribonucleotide having a translation enhancer and encoding a human erythropoietin, at Day 1 and Day 2 after transfection.

FIG. 9 is a bar graph showing the expression of EPO in Hela cells transfected with a circular polyribonucleotide having a translation enhancer and encoding a human erythropoietin, at Day 1 and Day 2 after transfection.

FIG. 10 is a bar graph showing in vivo expression of EPO encoded by a circular polyribonucleotide including a translation enhancer.

FIG. 11 is a graph showing the expression of EPO in A549 cells transfected with circular polyribonucleotides having spacer elements and/or translation enhancers and encoding a human erythropoietin with a CVB3 IRES, 24 hours after transfection.

FIG. 12 is a graph showing the expression of EPO in A549 cells transfected with circular polyribonucleotides having spacer elements and/or translation enhancers and encoding a human erythropoietin with an EMCV IRES, 24 hours after transfection.

FIG. 13 is a graph showing the expression of EPO in A549 cells transfected with circular polyribonucleotides having multiple spacer elements and multiple translation enhancers and encoding a human erythropoietin and a SARS-COV-2 RBD polypeptide, 24 hours after transfection of the cells.

FIGS. 14A and 14B are graphs showing the time course of expression of EGFPd2 in A549 cells transfected with circular RNAs having a spacer element and a translation enhancer and encoding a EGFPd2 polypeptide. FIG. 14A shows the expression of EGFPd2 over time. FIG. 14B shows the area under the curve from 0 to 24 hours.

FIGS. 15A-15C are bar graphs showing expression of EPO in A549 cells transfected with circular RNAs having a spacer element and a translation enhancer and encoding a human erythropoietin, 24 hours after transfection of the cells.

FIGS. 16A and 16B are bar graphs showing expression of EPO in A549 cells transfected with circular RNAs having a spacer element and a translation enhancer and encoding a human erythropoietin, 24 hours after transfection.

FIGS. 17A-17C are bar graphs showing HiBiT expression in HEK cells transfected with circular RNAs having a spacer element and a translation enhancer and encoding a Factor 9-Albumin-HiBiT polypeptide, 24 hours after transfection.

FIGS. 18A and 18B are bar graphs showing the expression of Gluc in Hela cells transfected with different concentrations of circular RNA having a spacer element and a minimized or full length elF4g aptamer translation enhancer and encoding a Gluc polypeptide, 48 hours after transfection.

FIG. 19 is a bar graph showing CFTR expression in HEK293T cells which were transfected with one of twelve (12) different circular RNAs encoding CFTR.

FIG. 20 is a bar graph showing polypeptide expression detected on the cell surface of HEK293T cells which were transfected with one of thirteen (13) different circular RNAs encoding the polypeptide.

FIG. 21 is a bar graph showing polypeptide expression detected on the cell surface of HEK293T cells which were transfected with one of four different circular RNAs encoding the polypeptide.

FIG. 22 is a graph showing the relative amount of circular polyribonucleotide present over time for circular polyribonucleotides having a 5′ spacer element and a 3′ RNA stability element and encoding a EGFPd2 polypeptide with a modified CVB3 IRES.

FIG. 23A-FIG. 23C are a series of bar graphs showing the concentration of polypeptide Factor 9-HiBiT encoded by circular polyribonucleotides having a 5′ spacer element and a 3′ translation enhancer and encoding a Factor 9-HiBiT, 24 hours post-transfection.

FIG. 24A and FIG. 24B are graphs showing the time course of expression data for EGFPd2 encoded by circular polyribonucleotides having spacer elements and/or translation enhancers and encoding EGFPd2 with a modified CVB3 IRES, where FIG. 24A shows EGFPd2 expression over time and FIG. 24B shows the area under the curve from 0 to 24 hours.

FIG. 25A and FIG. 25B are graphs showing the time course of expression data for EGFPd2 encoded by a circular polyribonucleotide having a 5′ translation enhancer and a 3′ spacer element and encoding EGFPd2 with a modified CVB3 IRES, where FIG. 25A shows EGFPd2 expression over time and FIG. 25B shows the area under the curve from 0 to 24 hours.

FIG. 26A and FIG. 26B are bar graphs showing the concentration of polypeptide B-HiBiT encoded by a circular polyribonucleotide having a 5′ spacer element and a 3′ translation enhancer and encoding polypeptide B-HiBiT with a EV69 IRES (FIG. 26A) or a modified CVB3 IRES (FIG. 26B), 24 hours post-transfection

FIG. 27 is a bar graph showing the concentration of polypeptide B-HiBiT encoded by circular polyribonucleotides having multiple translation enhancers or multiple spacer elements.

FIG. 28 shows the expression of polypeptide G encoded by circular polyribonucleotides having spacer elements and/or translation enhancers and encoding polypeptide G with a modified CVB3 IRES, 24 hours post-transfection.

FIG. 29A and FIG. 29B are bar graphs showing the area under the curve for GFPd2 (FIG. 29A) and concentration of polypeptide B-HiBIT (FIG. 29B) wherein the polypeptide is encoded by a circular polyribonucleotide having a single spacer design or a dual spacer design.

FIG. 30A and FIG. 30B are bar graphs showing the area under the curve for GFPd2 encoded by circular polyribonucleotides having a single spacer design or a dual spacer design.

FIG. 31A and FIG. 31B are bar graphs showing the area under the curve for polypeptide B-HiBiT encoded by circular polyribonucleotides having a single spacer design or a dual spacer design.

FIG. 32A-FIG. 32D are bar graphs showing the area under the curve for GFPd2 encoded by circular polyribonucleotides having a spacer element(s) and/or a translation enhancer(s), wherein the translation enhancer is FcIgG (FIG. 32A), TP5313 (FIG. 32B), LSP1 (FIG. 32C), or Histone4E (FIG. 32D).

FIG. 33A-FIG. 33D are bar graphs showing the concentration of polypeptide B-HiBiT encoded by circular polyribonucleotides having a spacer element(s) and/or a translation enhancer(s), wherein the translation enhancer is FcIgG (FIG. 33A), TP5313 (FIG. 33B), LSP1 (FIG. 33C), or Histone4E (FIG. 33D).

FIG. 34A-FIG. 34D are bar graphs showing the area under the curve for GFPd2 encoded by circular polyribonucleotides having a translation enhancer, wherein the translation enhancer is TBR16 (FIG. 34A), 12S (FIG. 34B), TRAM1 (FIG. 34C), and GPX4 (FIG. 34D).

FIG. 35A-FIG. 35D are bar graphs showing the concentration of polypeptide B-HiBiT encoded by circular polyribonucleotides having a translation enhancer, wherein the translation enhancer is TBR16 (FIG. 35A), 12S (FIG. 35B), TRAM1 (FIG. 35C), and GPX4 (FIG. 35D).

### DETAILED DESCRIPTION

The present disclosure features compositions including polyribonucleotides having one or more expression augmenting elements or spacer elements. The polyribonucleotides described herein are particularly useful in increasing the stability and/or increasing the expression of a polynucleotide cargo (e.g., encoding a gene or protein) encoded by the polyribonucleotide.

The disclosure provides polyribonucleotides including a first post-circularization element; a first expression augmenting element having a length of at least 100 ribonucleotides; a polyribonucleotide cargo; a second expression augmenting element; and a second post-circularization element; and wherein the first post-circularization element and the second post-circularization element together form a circularization junction. The disclosure also provides polyribonucleotides having a first post-circularization element; a first spacer element having a length of at least 100 ribonucleotides; a polyribonucleotide cargo; a second spacer element; and a second post-circularization element; and wherein the first post-circularization element and the second post-circularization element together form a circularization junction. The disclosure also provides polyribonucleotides having a first post-circularization element; a first spacer element; a polyribonucleotide cargo; a second spacer element having a length of at least 100 ribonucleotides; and a second post-circularization element; and wherein the first post-circularization element and the second post-circularization element together form a circularization junction.

The polyribonucleotide constructs described herein may show increased stability, potentially increasing the length of time the polyribonucleotide may persist before degradation. Additionally, the polyribonucleotide constructs described herein may show increased expression of a polynucleotide cargo encoded by the polyribonucleotide. Each of the DNA sequences described herein include the RNA equivalent sequence, as would be understood by one skilled in the art. Likewise, all RNA sequences described herein include the DNA equivalent sequence, as would be understood by one skilled in the art. The molecules, methods of producing, and uses thereof are described in more detail below.

### Circular Polyribonucleotide Elements

The circular polyribonucleotide may include from 5′ to 3′ a first post-circularization element; one or more expression augmenting element; a polyribonucleotide cargo; and a second post-circularization element. In some embodiments, the circular polyribonucleotide includes from 5′ to 3′ a first post-circularization element; a polyribonucleotide cargo; one or more expression augmenting element; and a second post-circularization element.

In some embodiments, the circular polyribonucleotide includes from 5′ to 3′ a first post-circularization element; a first expression augmenting element; a polyribonucleotide cargo; a second expression augmenting element; and a second post-circularization element.

In some embodiments, the circular polyribonucleotide includes from 5′ to 3′ a first post-circularization element; a spacer element; a polyribonucleotide cargo; an expression augmenting element and a second post-circularization element. In some embodiments, the circular polyribonucleotide includes from 5′ to 3′ a first post-circularization element; an expression augmenting element; a polyribonucleotide cargo; a spacer element; and a second post-circularization element. In some embodiments, the circular polyribonucleotide includes from 5′ to 3′ a first post-circularization element; a first expression augmenting element; a polyribonucleotide cargo; a second expression augmenting element; and a second post-circularization element. In some embodiments, the circular polyribonucleotide includes from 5′ to 3′ a first post-circularization element; a first spacer element having a length of at least 100 ribonucleotides; a polyribonucleotide cargo; a second spacer element; and a second post-circularization element.

The circular polyribonucleotides as disclosed herein comprise a first post-circularization element; (b) a first spacer element having at least 100 ribonucleotides (e.g., at least 100, 120, 140, 160, 180, 200, 250, 300, 400, or 500 ribonucleotides). The circular polyribonucleotide disclosed herein also includes a polyribonucleotide cargo and a second spacer element. In some embodiments, the second spacer element has a length of at least 100 ribonucleotides (e.g., at least 100, 120, 140, 160, 180, 200, 250, 300, 400, or 500 ribonucleotides) ribonucleotides. For example, the second spacer element may have a length of between 120 and 500 (e.g., between 120 and 400, 120 and 300, 120 and 200, 120 and 150, 150 and 500, 200 and 500, 250 and 500, 300 and 500, 400 and 500, or 350 and 500) ribonucleotides. In some embodiments, the second spacer element has a length of between 100 and 300 (e.g., between 100 and 280, 100 and 260, 100 and 240, 100 and 220, 100 and 200, 100 and 180, 100 and 160, 100 and 140, 100 and 120, 120 and 300, 140 and 300, 160 and 300, 180 and 300, 200 and 300, 220 and 300, 240 and 300, 260 and 300, or 280 and 300) ribonucleotides. In some embodiments, the second spacer element has a length of between 200 and 500 (e.g., between 200 and 450, 200 and 400, 200 and 350, 200 and 300, 200 and 250, 250 and 500, 300 and 500, 350 and 500, 400 and 500, 450 and 500, 300 and 500, or 300 and 400) ribonucleotides.

### Expression Augmenting Elements

The polyribonucleotides described herein may include one or more expression augmenting elements. In some embodiments, the circular or linear polyribonucleotides include between 1 and 5 expression augmenting elements (e.g., 1, 2, 3, 4, or 5 expression augmenting elements). For example, the circular or linear polyribonucleotides may include a first expression augmenting element and a second expression augmenting element. The one or more expression augmenting elements may include a translation enhancer, a stability element, a translation enhancer fused (e.g., joined, connected) to a spacer element, or a stability element fused (e.g., joined, connected) to a spacer element. In some embodiments, circular or linear polyribonucleotides includes a translation enhancer and a stability element.

The polyribonucleotides described herein may include the combinations of elements as described in Table 1 in 5′ to 3′ order. One or more spacer elements may be included between any one the elements described in Table 1.

TABLE 1
Exemplary Constructs

| 1 | 2 | 3 | 4 | 5 |
|---|---|---|---|---|
| Post circularization | Translation | Polyribonucleotide | Translation | Post circularization |
| element | enhancer | cargo | enhancer fused to | element |
| | | | spacer element^{†} | |
| Post circularization | Translation | Polyribonucleotide | Translation | Post circularization |
| element | enhancer | cargo | enhancer | element |
| Post circularization | Translation | Polyribonucleotide | Stability element | Post circularization |
| element | enhancer | cargo | | element |
| Post circularization | Translation | Polyribonucleotide | Spacer element | Post circularization |
| element | enhancer | cargo | | element |
| Post circularization | Translation | Polyribonucleotide | None | Post circularization |
| element | enhancer | cargo | | element |
| Post circularization | Translation | Polyribonucleotide | Stability element | Post circularization |
| element | enhancer | cargo | fused to a spacer^{†} | element |
| Post circularization | Translation | Polyribonucleotide | Translation | Post circularization |
| element | enhancer fused to | cargo | enhancer fused to | element |
| | spacer element^{†} | | spacer element^{†} | |
| Post circularization | Translation | Polyribonucleotide | Translation | Post circularization |
| element | enhancer fused to | cargo | enhancer | element |
| | spacer element^{†} | | | |
| Post circularization | Translation | Polyribonucleotide | Stability element | Post circularization |
| element | enhancer fused to | cargo | | element |
| | spacer element^{†} | | | |
| Post circularization | Translation | Polyribonucleotide | Spacer element | Post circularization |
| element | enhancer fused to | cargo | | element |
| | spacer element^{†} | | | |
| Post circularization | Translation | Polyribonucleotide | None | Post circularization |
| element | enhancer fused to | cargo | | element |
| | spacer element^{†} | | | |
| Post circularization | Translation | Polyribonucleotide | Stability element | Post circularization |
| element | enhancer fused to | cargo | fused to a spacer^{†} | element |
| | spacer element^{†} | | | |
| Post circularization | Stability element | Polyribonucleotide | Stability element | Post circularization |
| element | | cargo | | element |
| Post circularization | Stability element | Polyribonucleotide | Translation | Post circularization |
| element | | cargo | enhancer fused to | element |
| | | | spacer element^{†} | |
| Post circularization | Stability element | Polyribonucleotide | Translation | Post circularization |
| element | | cargo | enhancer | element |
| Post circularization | Stability element | Polyribonucleotide | Spacer element | Post circularization |
| element | | cargo | | element |
| Post circularization | Stability element | Polyribonucleotide | None | Post circularization |
| element | | cargo | | element |
| Post circularization | Stability element | Polyribonucleotide | Stability element | Post circularization |
| element | | cargo | fused to a spacer^{†} | element |
| Post circularization | Spacer element | Polyribonucleotide | Spacer element | Post circularization |
| element | | cargo | | element |
| Post circularization | Spacer element | Polyribonucleotide | Stability element | Post circularization |
| element | | cargo | | element |
| Post circularization | Stability element | Polyribonucleotide | Stability element | Post circularization |
| element | fused to a spacer^{†} | cargo | fused to a spacer^{†} | element |
| Post circularization | Stability element | Polyribonucleotide | Stability element | Post circularization |
| element | fused to a spacer^{†} | cargo | | element |
| Post circularization | Stability element | Polyribonucleotide | Translation | Post circularization |
| element | fused to a spacer^{†} | cargo | enhancer fused to | element |
| | | | spacer element^{†} | |
| Post circularization | Stability element | Polyribonucleotide | Translation | Post circularization |
| element | fused to a spacer^{†} | cargo | enhancer | element |
| Post circularization | Stability element | Polyribonucleotide | Spacer element | Post circularization |
| element | fused to a spacer^{†} | cargo | | element |
| Post circularization | Stability element | Polyribonucleotide | None | Post circularization |
| element | fused to a spacer^{†} | cargo | | element |
| Post circularization | Spacer element | Polyribonucleotide | Translation | Post circularization |
| element | | cargo | enhancer fused to | element |
| | | | spacer element^{†} | |
| Post circularization | Spacer element | Polyribonucleotide | Translation | Post circularization |
| element | | cargo | enhancer | element |
| Post circularization | Spacer element | Polyribonucleotide | Stability element | Post circularization |
| element | | cargo | fused to a spacer | element |
| | | | element^{†} | |
| Post circularization | None | Polyribonucleotide | Stability Element | Post circularization |
| element | | cargo | | element |
| Post circularization | None | Polyribonucleotide | Translation | Post circularization |
| element | | cargo | enhancer fused to | element |
| | | | spacer element^{†} | |
| Post circularization | None | Polyribonucleotide | Translation | Post circularization |
| element | | cargo | enhancer | element |
| Post circularization | None | Polyribonucleotide | Stability element | Post circularization |
| element | | cargo | | element |
| Post circularization | None | Polyribonucleotide | Stability element | Post circularization |
| element | | cargo | fused to a spacer^{†} | element |

^{†}The spacer element may be fused to the 5′ end or the 3′ end of the Translation Enhancer or Stability Element

### Translation Enhancers

The polyribonucleotides described herein may include a first expression augmenting element or a second expression augmenting element. The first expression augmenting element may include a translation enhancer. The second augmenting element may include translation enhancer. In some embodiments, a circular or linear polyribonucleotide described herein includes one or more translation enhancers. In some embodiments, a translation enhancer may be fused (e.g., joined or connected) to a spacer element. Translation enhancers may be present in between any of the nucleic acid elements described herein. Translation enhancers may also be present within a nucleic acid element described herein. In some embodiments, a translation enhancer is a spacer element. In some embodiments, a translation enhancer is a stability element.

In some embodiments, the translation enhancer is from a gene encoding an RNA binding protein. In some embodiments, the translation enhancer comprises nucleic acid sequence comprising a fragment from a gene encoding an RNA binding protein. In some embodiments, the translation enhancer has at least 85% (e.g., at least 85%, 86%, 87%, 88%, 89%, 90%, 91%, 92%, 93%, 94%, 95%, 96%, 97%, 98%<99%, or 100%) sequence identity to a nucleic acid sequence comprising a fragment from a gene encoding an RNA binding protein. In some embodiments, the translation enhancer is from a gene encoding a BYDV like-element (BTE), a translation enhancer element (TED), a PMV/PEMV-like translation enhancer (PTE), an I-shaped structure (ISS), a Y-shaped structure (YSS), a t-shaped structure (TSS), dumbbell shaped structure, viral RNA UTRs (including Dengue, West Nile, Zika, Rotavirus), EMCV, CVB3, hepatitis B virus posttranscriptional regulatory element, human genomic fragments, a histone mRNA sequence, a cyclin D mRNA sequence, or an elF4g aptamer sequence. In some embodiments, the translation enhancer element is from a plant virus. In some embodiments, the translation enhancer comprises nucleic acid sequence comprising a fragment from a plant virus. In some embodiments, the translation enhancer has at least 85% (e.g., at least 85%, 86%, 87%, 88%, 89%, 90%, 91%, 92%, 93%, 94%, 95%, 96%, 97%, 98%<99%, or 100%) sequence identity to a nucleic acid sequence comprising a fragment from a plant virus. In some embodiments, the plant virus is a Barley yellow dwarf virus (BYDV) like-element (BTE) translation enhancer. Non-limiting examples of plant viruses with BTEs include BYDV, TNVD, OLV1, LWSV, SCNMV, CRSV, TBTV, GRV, OMMV, BBSV, RSDaV, and OPMV. BTEs bind to elF4g with high affinity. In particular embodiments, a circular or linear polyribonucleotide comprises a BTE sequence described in TABLE 2. In some embodiments, the BTE comprises a sequence having at least about 80%, 85%, 90%, 95%, 97%, 98%, 99%, or 100% sequence identity to a sequence from TABLE 2, SEQ ID NOs: 1-22.

In some embodiments, the translation enhancer element is from a plant virus containing a TED. Non-limiting examples of plant viruses with TEDs include STNV, PLPV, PCRPV, ELV, RrLDV, PelRSV, and CbMV. TEDs bind to elF4F with high affinity. In particular embodiments, a circular or linear polyribonucleotide comprises a TED sequence described in TABLE 3. In some embodiments, the TED comprises a sequence having at least about 80%, 85%, 90%, 95%, 97%, 98%, 99%, or 100% sequence identity to a sequence from TABLE 3, SEQ ID NOs: 23-29.

In some embodiments, the translation enhancer element is from a plant virus containing a Panicum mosaic virus/Pea enation mosaic virus (PMV/PEMV)-like translation enhancer (PTE) translation enhancer. Non-limiting examples of plant viruses with PTEs SCV, PFBV, CarMV, HnRSV, PSNV, HCRSV, GaMV, CMMV, TPAV, JINRV, PEMV2, PMV, MCMV, BGLV, AdMV, and CLSV. PTEs bind to elF4E with high affinity. In particular embodiments, a circular or linear polyribonucleotide comprises a PTE sequence described in TABLE 4. In some embodiments, the PTE comprises a sequence having at least about 80%, 85%, 90%, 95%, 97%, 98%, 99%, or 100% sequence identity to a sequence from TABLE 4, SEQ ID NOs: 30-46.

In some embodiments, the translation enhancer element is from a plant virus containing anISS translation enhancer. Non-limiting examples of plant viruses with ISSs include MNeSV, MNSV264, CBV, MWLMV, JCSMV, and GoMVA. ISSs bind to elF4E bound to elF4G. In particular embodiments, a circular or linear polyribonucleotide comprises a TSS sequence described in TABLE 5. In some embodiments, the TSS comprises a sequence having at least about 80%, 85%, 90%, 95%, 97%, 98%, 99%, or 100% sequence identity to a sequence from TABLE 5, SEQ ID NOs: 47-53

In some embodiments, the translation enhancer element is from a plant virus containing a YSS translation enhancer. Non-limiting examples of plant viruses with YSSs include TBSV, CIRV, CymRSV, CNV, AMCV, PNSV, GALV, PLCV, PeLV, and LNV. YSSs bind to elF4F. In particular embodiments, a circular or linear polyribonucleotide comprises a YSS sequence described in TABLE 6. In some embodiments, the YSS comprises a sequence having at least about 80%, 85%, 90%, 95%, 97%, 98%, 99%, or 100% sequence identity to a sequence from TABLE 6, SEQ ID NOs: 54-63.

In some embodiments, the translation enhancer element is from a plant virus containing a TSS translation enhancer. Non-limiting examples of plant viruses with TSSs include TYMV, RCNM, TCV, and CCFV. TSSs bind to the 60s ribosomal subunit. In particular embodiments, a circular or linear polyribonucleotide comprises a TSS sequence described in TABLE 7. In some embodiments, the TSS comprises a sequence having at least about 80%, 85%, 90%, 95%, 97%, 98%, 99%, or 100% sequence identity to a sequence from TABLE 7, SEQ ID NOs: 64-67.

In some embodiments, the translation enhancer element is from a plant virus containing a dumbbell-shaped translation enhancer. A non-limiting example of a plant virus with a dumbbell-shaped translation enhancer includes CABYV-X. In particular embodiments, a circular or linear polyribonucleotide comprises a dumbbell-shaped translation enhancer sequence described in TABLE 8 SEQ ID NO: 68. In some embodiments, the dumbbell-shaped enhancer comprises a sequence having at least about 80%, 85%, 90%, 95%, 97%, 98%, 99%, or 100% sequence identity to a sequence from TABLE 8, SEQ ID NO: 68.

In some embodiments, the translation enhancer element is from a gene from a mammal. In some embodiments, the translation enhancer comprises nucleic acid sequence comprising a fragment from a mammalian gene. In some embodiments, the translation enhancer has at least 85% (e.g., at least 85%, 86%, 87%, 88%, 89%, 90%, 91%, 92%, 93%, 94%, 95%, 96%, 97%, 98%<99%, or 100%) sequence identity to a nucleic acid sequence comprising a fragment from a mammalian gene. The gene from a mammal may be, but is not limited to, histone or cyclin D mRNA sequences that binds to elF4E. In some embodiments, the translation enhancer element is from a synthetic sequence, including but not limited to, an elF4G aptamer that binds to elF4G. In some embodiments, the translation enhancer element is from a viral sequence. Non-limiting examples of translation enhancers from viral sequences are HCV and DENV. In particular embodiments, a circular or linear polyribonucleotide comprises a mammalian, synthetic, or viral translation enhancer sequence described in TABLE 8. In some embodiments, the mammalian, synthetic, or viral translation enhancer comprises a sequence having at least about 80%, 85%, 90%, 95%, 97%, 98%, 99%, or 100% sequence identity to a sequence from TABLE 8, SEQ ID NOs: 68-159, and 273-287.

In particular embodiments, a circular or linear polyribonucleotide comprises a translation enhancer element described in TABLE 2-8. In some embodiments, the translation enhancer comprises a sequence having at least about 80%, 85%, 90%, 95%, 97%, 98%, 99%, or 100% sequence identity to a sequence from TABLE 2-8.

In certain embodiments, the translation enhancer includes a nucleic acid sequence having at least 85% (e.g., at least 85%, 86%, 87%, 88%, 89%, 90%, 91%, 92%, 93%, 94%, 95%, 96%, 97%, 98%, 99% or 100% sequence identity) sequence identity with the nucleic acid sequence of any one of SEQ ID NOs: 1-183, 255, 256, 273-287, 290-303, or 305-333. In some embodiments, the translation enhancer includes a nucleic acid sequence having at least 90% (e.g., at least 90%, 91%, 92%, 93%, 94%, 95%, 96%, 97%, 98%, 99% or 100% sequence identity) identity with the nucleic acid sequence of any one of SEQ ID NOs: 1-183, 255, 256, 273-287, 290-303, or 305-333. In some embodiments, the translation enhancer includes a nucleic acid sequence having at least 95% (e.g., at least 95%, 96%, 97%, 98%, 99% or 100% sequence identity) identity with the nucleic acid sequence of any one of SEQ ID NOs: 1-183, 255, 256, 273-287, 290-303, or 305-333. In some embodiments, the translation enhancer includes a nucleic acid sequence having a nucleic acid sequence of any one of SEQ ID NOs: 1-183, 255, 256, 273-287, 290-303, or 305-333.

In some embodiments, the translation enhancer element may be TBR16, 12S, TRAM1, or GPX4.

The translation enhancer element may be a 5′ UTR or a 3′ UTR. In some embodiments, the 5′ UTR or 3′ UTR is a human translation enhancer. In some embodiments, the 5′ UTR or 3′ UTR comprises any one of the 5′ UTRs or 3′ UTRs described in Table 9, or a portion thereof.

The translation enhancer element may have between 20 and 750 ribonucleotides (e.g., between 20 and 700, 20 and 650, 20 and 600, 20 and 550, 20 and 500, 20 and 450, 20 and 400, 20 and 350, 20 and 300, 20 and 250, 20 and 200, 20 and 150, 20 and 100, 20 and 50, 50 and 100, 50 and 750, 100 and 750, 150 and 750, 200 and 750, 250 and 750, 300 and 750, 350 and 750, 400 and 750, 450 and 750, 500 and 750, 550 and 750, 600 and 750, 650 and 750, and 700 and 750 ribonucleotides). For example, translation enhancer may have a length of between 100 and 500 ribonucleotides (e.g., between 100 and 450, 100 and 400, 100 and 350, 100 and 300, 100 and 250, 100 and 200, 100 and 150, 150 and 500, 200 and 500, 250 and 500, 300 and 500, 350 and 500, 400 and 500, or 450 and 500 ribonucleotides).

TABLE 2
BTE sequences

| SEQ ID | | |
|---|---|---|
| NO: | Virus | Sequence |
| 1 | Barley yellow | GUCUUAGCAAGCUCUGAGCCAGGAGAUGGACAUAAACCAUAGCAAUCCAACGUGUAACC |
| | dwarf virus | GCAAUGGGGCAAACAACAGGUGAACCGUGUCCACGGGCCUGGUUACCGAAAGGAAAGC |
| | (BYDV) | CAGUAUCCAACACAGCAAUGUGUUGGGGGUCACACCUUCGGGGUACUCUUAACGCUGA |
| | | CACUCGAAAGAGCAGUUCGGCAACCC |
| 2 | Tobacco | UCGCUUUCAUAGAUCCGUCUUCCCAGAGACGUUAAGAAGAAGCUGGAGAAAAAUAUUAG |
| | necrosis | GUUAGAAGCUUGGGCGUGACAAACCCAAGUUGCAUCUCUUACGUGGUUAAUCACACUG |
| | virus (TNVD) | UAUGUUGACGAAUAGGCCGGAUCCUGGGAAACAGGUUUAACGGGCUCUCUGUGGUGGA |
| | | GGGCCGACGCAUCACCUAUUUGUGCUCCAACAGUGGUUGUCAUCACGUGUCCUGACAU |
| | | GGCUCCAUGCGACAGCAUGGGGGGGUCCAGAGUCAGUCCCCUCUUUAUUUACCUAGGA |
| | | UUUCCUAGGAACCC |
| 3 | Olive latent | CGUAACUGUGCUAAUCCCUAAAAGUUUUAGUAGAGCGGUAAGCAAUCCGGAUCCUGGUA |
| | virus | GACAGGUUUAACGGGCUGGGGGUGGUGCCCCGGCCGUCGCAUCACUUGCUGAUACAAU |
| | (OLV1) | CUACAAGACUCAUGGGAGGGUCUGCACAGGUAUCCACGCCGAGGACGGGACGAAAGUC |
| | | UCGGACGGGGGGUAGUGGACCCAACACCAGAUUGAGGGGCUUUUGCCCCACCCCA |
| 4 | Leek white | UCACCUCACGGUUUUGUUAAUUACAUUCAAGUCGAAGUUGCAUAGAUCCCGAGAACGGG |
| | stripe virus | UUUUAACGGGCUCCUGUGGUGGGAGCCGACGCUCAUCGCUUCUGAAAUUUGAAUGGUU |
| | (LWSV) | GUCACAAAACAACAUAUGAUGGCUCACUACAACAGUAGUGGGGGGUCCAGGCAUAUGUC |
| | | CCGCAAUACUUAUAGCUACCUUUUGGUAGUACCC |
| 5 | Sweet clover | UGCAAGCGUAACCUCCAUCCGAGUUGCAAGAGAGGGAAACGCAGUCUCGCCGACCCUG |
| | necrotic | UUGCCAAACAGUAAAAUUGGAAAAAAUAGAGUGUUAGGAGUAGUUCCCGUACCCGCGGG |
| | mosaic virus | ACAAGAACCUAUUGUAGUAGACGAACCGGUCUCGGACCCUGGUAAACAGGUACCUAGCG |
| | (SCNMV) | UAAUAGAAGGUCGCUAGAAAUCGGGUGGGCUUCACCUACGGGUAACCCACCGUCGAGA |
| | | GGGAAAACCUCUGUGUGGUUGUGCGCACGUCGUUUCCUGAAGAAUCUACCUUCGGUUA |
| | | AACAAAAUUGGCCCCUGAGAGUAGAAAACUCAGGUUAAUAAAACAGGAGAUUAUUUGCU |
| | | UAGUCGUAUAACGGCUAAGCACCCC |
| 6 | Carnation | AUUGGACACAAAACGGAGCUAGAUUUAGUGUUGCAAUUGCUAGACCGUUAGCCACUACG |
| | ringspot virus | CCAGACAGUGCGAUAACUGAGGUCGUCUAUGACCCACAGGGUGCCAUCCUGUGCAAUU |
| | (CRSV) | UCCAACCCUUGGCAGGGGUGAUCCUAGAGGACGCGGGCAUUGACUUGCCUGGGGAUGC |
| | | UUCUAGUUACAUACCGGAGUGGUUGAUAGACCUCUGUGUAAGGAAACAGGUGUGAGAC |
| | | UGGAUCCUGGGAAACAGGCUGGGAUGGUCCCGUAAGUUCGGGCGGUGCCCUUCGGGG |
| | | GUAACCCCACCGCCGACGUAUCACCUCACACGCUGUUGGCCGAUAAGCCGAGGUGUUG |
| | | GAACCGCACCACUCUGGAGGUUCCCUCUCCUUAUGAGGUGGAGGCACAGCACGCCUUA |
| | | AGUGUCUGCGAUCCGGCGUUCACGUGCCGGUGGUCCCCCUAGUUAACGUGGUGCCACA |
| | | GUCUGUGGUAGGGGCAGCUGGUCGGAUUCCAGCAGAAACCAAGUAGCAAAUGGACGUU |
| | | GCUACCUCGAGCCUAGAGUCAUGGGCCCGCAUCGACGUUUGCGGUAACUCACAGCGGG |
| | | CGAUAAUAGCUGUUGAGAAUGUCGGUUCCUGACUUAGCGGGAGAUGAGCACUUUCUCU |
| | | CGCGCCC |
| 7 | Tobacco | AUUGGACACAAAACGGAGCUAGAUUUAGUGUUGCAAUUGCUAGACCGUUAGCCACUACG |
| | bushy top | CCAGACAGUGCGAUAACUGAGGUCGUCUAUGACCCACAGGGUGCCAUCCUGUGCAAUU |
| | virus | UCCAACCCUUGGCAGGGGUGAUCCUAGAGGACGCGGGCAUUGACUUGCCUGGGGAUGC |
| | (TBTV) | UUCUAGUUACAUACCGGAGUGGUUGAUAGACCUCUGUGUAAGGAAACAGGUGUGAGAC |
| | | UGGAUCCUGGGAAACAGGCUGGGAUGGUCCCGUAAGUUCGGGCGGUGCCCUUCGGGG |
| | | GUAACCCCACCGCCGACGUAUCACCUCACACGCUGUUGGCCGAUAAGCCGAGGUGUUG |
| | | GAACCGCACCACUCUGGAGGUUCCCUCUCCUUAUGAGGUGGAGGCACAGCACGCCUUA |
| | | AGUGUCUGCGAUCCGGCGUUCACGUGCCGGUGGUCCCCCUAGUUAACGUGGUGCCACA |
| | | GUCUGUGGUAGGGGCAGCUGGUCGGAUUCCAGCAGAAACCAAGUAGCAAAUGGACGUU |
| | | GCUACCUCGAGCCUAGAGUCAUGGGCCCGCAUCGACGUUUGCGGUAACUCACAGCGGG |
| | | CGAUAAUAGCUGUUGAGAAUGUCGGUUCCUGACUUAGCGGGAGAUGAGCACUUUCUCU |
| | | CGCGCCC |
| 8 | Groundnut | AACUACGACCCCGCCAGGGGUUAAUUCUACCACAGGUUGGCAGCUGUGGGAUCCCAGU |
| | rosette virus | CCAGGUGUCUUUAACUAGAUGUGGAUGGACUGGUUGAUUUUGUCGGCCUUGAUAGGGG |
| | (GRV) | ACGAAACCACGAGUCAAGCGGAAGAGAUAGGGGGUGUUGGAUCCCGGGAAACUGGCAG |
| | | CUGGGCAGCGUAAGCACGGGUGUGUGAACCCCACACCGACGUAUCACCCCCCUUGGAC |
| | | UUAGUACGCGAUUUGAGGCGGUUGUUCCACGUGGGAAGCAGGCUCGGCACCCAAUCCG |
| | | ACAGGGAAAAGUCGUAGGCUCCGGCGCUGCGGGAGGGUAAACUAGCGGCCGAUACGCA |
| | | UUGUGCAGUGCGUUGGUCCCACUCAGCACAUGGCGCUCUGCCAUAGGGACGGAAGGGC |
| | | CGUGGUACUCCCUUCAACAGGCGGCACCCAGUGAGGCUCGCCAAUCCAUAUGACUAGG |
| | | AUUAUGCUGAGUGAUACCCCCUUUCGAACGUACGGACGUCCAGCUGAGAUCUCCUAGA |
| | | UAACAUGACACCC |
| 9 | Olive mild | GUGUUAGUUAACUGGCUUAAACCCUAAGGGUAGAUAUGUGGGCGGUAAGCAAGAGGGA |
| | mosaic virus | UCCUGGGAAACAGGCUUCGACGGGUUGGGGGUGGUGCCCCGGCCGACGCAUCACUUG |
| | (OMMV) | CUGAUACAACCAUUAGACACCUAAGGGCGGGUCUAGCCAGGUCUCCACGCCAUGAUCAA |
| | | UUGGAAACGAUUGUGAGGGGGGUAGUGGAACCCAUACCAGAUUGAGGGGCCUUUGCCC |
| | | CACCCC |
| 10 | Broad bean | AUCCCACAUCCUGGUGUGGUUAAUCCAGUGAAAAAUAUUAGGAAAUCCUUGGGAUUCAC |
| | stain virus | ACCCCAGGGAGGAUUGCUUGCAUAGUGCAGGUAUGUUGAGUUACACUAUGAGGUAUUG |
| | (BBSV) | GUGCUGAAUCCUGGUAAACAGGCUUGACAGGUUUGGGUGGUUCCAGACCGAUGUAUUA |
| | | CCCAAGAUACUCAUGGUACUCUACUAGAAAACACUAUGCGUGCGCACACGGCUGGCUAU |
| | | GUCCUAUCAUAGCUGGGGGCCCCGGAGUGCGAAACCCUCUUAUAUACCUGGUCUUCCA |
| | | GGUGCCC |
| 11 | Rose spring | GGAGCCAAUUCUAGGCUUCGCAGUGCUGGAUCCUGGUAAACAGGCGUUCAAGUAACCU |
| | dwarf- | AAGCUUCGGUGAAGGGCAAUGCUUGUUGUAAGCUCGGGAGGUCUUGUCAACCUCCGUC |
| | associated | GUAUCACCUGCGUACCUCCCCUGGAUAAUAACCCAUUUCUGCACCAGACUA |
| | virus | |
| | (RSDaV) | |
| 12 | Opium poppy | UAAGCUGACUGACAUUCGGUUGUUGCGACGCUUAGUUAUAACAUGGGUGGGUUUAGAG |
| | mosaic virus | UUGCAAUAUAGUGGGAGCCGGGUCCUGGUAAACAGGUUGAGUUGGUCUCGUAAUCUCG |
| | (OPMV) | GGUGGACUGCCAAUCCACCGUCGUACCACUCUCAUGGGCUAUUGCAUCCUCUAAUAGG |
| | | GAGUUUCAGGAACACCCA |
| 13 | Soybean | UAUAGGUAGAAGUGUGUUUGCGCUUUCCAUCACACGAAAUUGGAGCCAACCAUGGUGA |
| | dwarf virus | GUUGCUGUUGUGGGAUCCUGGGAAACAGGUUCGGUGAACAAAAAGCCGGUAAUCUCGG |
| | | GCGUUUGUCAGACGCCGCCGAAUCAACACAGUAUGCAUAUCCAUCAAUAACCCAAGUUA |
| | | CGUGUGACUAAAAAUUAGAAUUAGUUUACCGCCUCUCUUGUAAACCACAUACAAGACGU |
| | | GAGAUUGUGGAGAGUAUUAUCCCUUGAAUAAGGAUAAUUGAAAGUUUCUGGUUUCAAUC |
| | | CAUUGAUCAAAAUUGACACUGCUUCUGGUGAUUACACUGCCGCUGGUGAUACAGCGUAA |
| | | ACAAUUUCUGCUUUUCGCUCUAUGAAAUGAGAGUGAUUAGUAAAGUCUUGCCGUUCACA |
| | | UGUUUAUUUGACGGUACCAGGCUUCGCCCCUUUGAUGGCGUUAAAUGAAAGCGGAGUA |
| | | AACCGACACCACCUUACCAGGUCGGGUUGACCUGGGGAAACCGGGAAAAUACCCGGGA |
| | | CACUUGGUUUAGUGGAGUUUAAACUCGAGGGCUCUUCCUCGUUAAAAAGGGGGUAGCC |
| | | AGGUUGAAAACUGGCACUAGGGAGAAAUCCCUCUUUGUUGUUAAGGUGGCUUUGUGUC |
| | | CACCUGCCCC |
| 14 | Red clover | UUGGUUCUUUUAAGUGUAGCCUCCACCCGAGUUGCAAGAGGGGAACACGCAGUCUCGC |
| | necrotic | CGACCCUGUUGGCAAACAGUAAAAUUGCAAAAAAUAGAGUGCUAGGAGUAGUUCCCGUA |
| | mosaic virus | CCCGCGGGAGCAAGACCCUACUACAGUAGACGAACCGGCAUCGGACCCUGGGAAACAG |
| | RNA1 | GUACCUAGCGUAUUAAUAGGUCGCUAGAAAUCGGGCGGGCUUCACCUUCGGGUAACCC |
| | (RCNMV) | GCCGUCGAGAGGGAAAACCUCUGUGUGGUUGUGCGCACGUCGUUGUCGGAAGACUCUC |
| | | ACUGUUGUAGUUAUUUCCUUUUUCUUUUAGGUAGGAGCACUUGCUCAGAGGGCGCAUA |
| | | CCUCUGGUAAAACAAAAUUGGCCCCUGAGAGUAGAAAACUCAGGUUAAUAAAACAGGAG |
| | | AAUAUUUACCUAGUCGUAUAACGGCUAGGUACCCC |
| 15 | Bean leafroll | CCACUAGUGCAUCGAAUCCUGGGAAACAGGCACGGAGAACAAAAUCCGGUAAGCUCGGG |
| | virus | AAGCCGUCAACUUCCGCCGUAUUGAUGAAAAC |
| | (BLRV) | |
| 16 | Tobacco | ACACCAUUGGAGACGGAGACGGAUCCUGGGAAACAGGCUUGACGGGCGGGGGGUGGU |
| | necrosis | GCCCCCGACGACGCAUCACUCCGGAUACCAAUGGU |
| | virus | |
| | (TNV-A) | |
| 17 | Ethiopian | ACUAACACUGCCCGGGAUGGCAAUAAACUACUGUGCGUCCGCAAUGACGUUAAACUACG |
| | tobacco | GCAGUCUGCAAUGACUAUAAACCUACGGUCCUGCCAAAGGACUACUCUUUUGGGGUUG |
| | bushy top | GCAUCCCAAGAUCCCACACCCGGUGUCUUUAGCUAGACGUGGGUGGUGUGGUUACAAU |
| | virus | UUGACAGCCUCGGAGUGGCGAUACAGAGAGACUAACUGAUAAAGUGGGCUAGGGCGGA |
| | (ETBTV) | UCCUGGGAAACAGGCUGCCUGGCGGCGUAAGCUCGGGUGUGUGAACCCCACACCGACG |
| | | CAUCACCUAGCCGGGACUUGACACGGUAGUUUCACUUAUUCUCACAUGGACAGGGUCU |
| | | GUCACGUUCCCGACUACGUAAAGUCGUAGACUCCAGCGCUGCGGGAGUAUAAUUUCGC |
| | | GGCCGACGUGCAUUGCUCGGUGCACUGGUCUCCCCUAGAGCCUAACGGUCUGUUAGUG |
| | | GGAUAUGACCCAUGGUGGGUCGUACCGGGUGGUAACCGGAAUAACAACCAACCAAGAC |
| | | UGGCCAUCCUAGGCUGGAGUACCAUGCGACUCCCAAACCUACUUCGGGCGUAUAAUAG |
| | | AAGUGGUAAUGCAUGGAUUAGCUAAUAGCGGCGGGAAUUCACCCUCGUCGCGCCC |
| 18 | Rose spring | GGAGCCAAUUCUAGGCUUCGCAGUGCUGGAUCCUGGUAAACAGGCGUUCAAGUAACCU |
| | dwarf- | AAGCUUCGGUGAAGGGCAAUGCUUGUUGUAAGCUCGGGAGGUCUUGUCAACCUCCGUC |
| | associated | GUAUCACCUGCGUACCUCCCCUGGAUAAUAACCCAUUUCUGCACCAGACUA |
| | virus | |
| | (RSDaV) | |
| 19 | Ixeridium | CCAGGAGCAGCAAAACCCAGCCCCGGAGAACUUGCAAGAGGGGGGGGGUAAAGUGUUU |
| | yellow | GUCCGCGUGAGUGCCCUUACGUGUGUCUCGGGGCCUAGCCAGCCCACUCUAACUCCAC |
| | mottle- | CCUUAGGCAAGGUGGCGCACUGUCGGCGUGCGUAUUUCCCGCUGGGUUGACAACCAGC |
| | associated | AGACCCUACCUGGUCGGGGCUUAACUGACUUUCCCAGGUAGUUACUUUAGUGUGGUGG |
| | virus 2 | AGAUUGCUGGGUAUAGGUGCUGGAUCCUGGGAAACAGGUUUAACGGGCGGACUAGUCA |
| | (IxYMaV2) | CCCGCCGUCGCAUCACCCUUGAUACCAGCUCCUCCUCACCGUGGGGUGGGGAACACAC |
| | | UUGCAGUAUAUAGUUAUGCCAAAAAUAUUCAGCCAACCCAGGUUGGCCCACUGUAUUGU |
| | | CCGGUGCAGGGCC |
| 20 | Patrina mild | CAACCCCAACAAACUCAGCCGGGAAGAACUUGCAAGAGCCGGAGAGGUAGCGUUUACUC |
| | mottle virus | CAUGUCUAGCUGGCGCAUGGUUUUAGUGUACAUACAAAUCCAGGGAUCAGCGCCUGGC |
| | (PatMMoV) | GGCACACCACUUGUCGCAACUCAGUGGGGAUCCCAUGGAUAGGCACUUAGAUGAGUCG |
| | | AGUCCAUGGUUGAGUUUGGGUGUCUUGAAGGAUCGGUUGGUCUUGAGAUUGAGGCGU |
| | | GAGUGUGCUGGAUCCUGGGAAACAGGUUUAACGGGCGACUAGUCACCGCCGACGCAUC |
| | | ACCACAAUAUAGCCUCUCAAGUGUGGGCACGGGACCCACAGCCAACAGAGGGAAGCAGA |
| | | CAUCCUGUUUACAAAAACCAAAAAUAGCUUGGUGACCCAGGUCACCCCCCCUCCAUUGU |
| | | CGGGUGGUUGGGGCCUUCUCACGACCAUGUGCAAUAUUCAGCACAUGUGGGUGUGGUA |
| | | CACGGUAUGGCCACAAGGGAGAAAACCCUCCAAAUCCGUUGACCGUCGUCAGGUCACC |
| | | GACCUGCUGGUCUUAGCAGGCGUUGGAUACGUACAUCCCUGGAGCAGCACCAGAAAGU |
| | | CCCGGGCAUUACAAUAGGGAUAAAGCUGUGAGAUCGUAUUUUAGCGUCGAGUGCAGCA |
| | | CUCGGCGCGCCC |
| 21 | Paederia | ACUGAAACCCCAAGCCGGAGGAACUGUUGCAACUGCCGGAAUGGAAGUCGUUCAAGCC |
| | scandena | CGUGAAGGCUUAUAAACUAAAUGGCCACAGAAGGCAACUGUGGACAGAAAUUGUAUAGA |
| | chlorosis | CACGGAGUAAAUACCGUGGUGCCGACCAACUCGGCUGCAUCUCUUGCUGGUGUGGACU |
| | yellow virus | ACCAGUGACCCCGGGGGCGGAUCCAUUAAGUUGGCUUGGGAUCCCCCGGUUAAAUAGG |
| | (PSCYV) | AACGGUAGUUGGCUUAGACGGAAUGGUUGGAGUGGAAAACCUGAUGCUGGAUCGCCUC |
| | | CUGGGAAACAGGCAGCAUGGCGCAUAAGAUCGGGGGAGGUUGGCCCGCCUCUCCGACG |
| | | UAGGGUACCAGCAUUGAGAAGGAGUACAACCACCUUAGUACGUCGAAGCUUGGAUAAGA |
| | | CUGUUUCCACCCUACCCACAGGGUUACAGUGGUAGAGGACUGUGGGCUUUUCCCACAG |
| | | CGAUUGGAUUAGUGUGUCCAUGGUCUCCCAAGUCAACACUUUCGCCUUGUGCGUAUGG |
| | | GAGUGGGUUGGUUUCCCCACACGUCUCCACAGACGUUAAACCUGGCCGUCCACCAAGA |
| | | CGUUAAACUAGGAACCGGUUGGUGACCGUCAACACACCCUGGCAGAGGACGCCAGAGC |
| | | GACUCCCGGGCAUUAUAAUAGGGACUCGCCAAGUUCUCAUCUGUUCUUAGCGGUUGUU |
| | | AUCACUGCAACCGCGCCCG |
| 22 | Picris | GGCUCCCCAUCCUUCGGGGCUUGAAGGACGUUGGACGCGAUAAGUCUCCAGAGUGGAU |
| | umbravirus | CUGGCAGCCCCCAGGGCUAUAAUAUGGGGGAAUCCACCAGUUUGAACCUAGCGAGCAG |
| | (PIUV) | CCUAAGAAGGUACUUGUUCGAUCUGGUAAUGUUAU |

TABLE 3
TED sequences

| SEQ ID | | |
|---|---|---|
| NO: | Virus | Sequence |
| 23 | Satellite tobacco | UAAUCCCAGAGGUUCACAAUGUUAGUGAUGGGCGCUGAAAGAUGCGUAGCUACCCUU |
| | necrosis virus | CUGGAGCCACUUCCUGGUGGUAAGCAGAAAUCCAAGGGUACGGUGGUACGGUGGAAA |
| | (STNV) | GCAGUCCCAGCUCUGCAUUGGGAACCGGCUCACACCCAGCUUAGGGCUAAAGUGUAC |
| | | UACUUGCUCAUUUGUAGUCUAAAUGAGACGUUGGCCUCGCCGUGUCGAGGUGGCCU |
| | | AAGGGAUUGGAACCCCUGAUGGUCGUAGUCGAAUUUCCCGUGUUUCAUCCCGAGUCU |
| | | CUUGGUCAUAAUGCCAUUAGUAGGUCAAGCACCCAACGUAACUUCAAAGAUAUCCUCC |
| | | UUGCAACAAGAAUAUGUGCGCUGUCUGUGUUUAAAGCGGUAUAUUUUGGGCGCCGGC |
| | | AUAUCGUUGCUUGGACCAGGGCCCCACGCCGGUUGGUACCCGGGUGGCUUCCCCUC |
| | | GUUCACAGGGCUUUAGGAGAUGAUAAGGUAUAGUUACUAGGCAAAUGCGGACAAACC |
| | | UGAAAAGCUCGCUAGUGGUGGGCUGGCCAAGCGAAGAACCUCAUCCAGGUAUAGUUC |
| | | UACAUGGGAAAUUUGGUACCAUCCAAACUUCUAUGAAGUCCUCGACUAACCCC |
| 24 | Pelargonium line | AGCUUACAUCAACAAGCUGUGAGGAAAGCCACUGCGGUGGGGUCAGUCUCCUGGUGC |
| | pattern virus | GCCAACACCCAGAAGGGGAGACAACUUCAGUGGUUCAAAACCCGAUGCUUCGUGUAA |
| | (PLPV) | GCCUAGAAAAAUCCAAAAACAGAGUGCUACUGCUUUCAGUGUGCGAGCUUUAUUUGC |
| | | UCACUUCCUCCGUCGAUAAAAUCCCGAAUUUUCGGGUGGGCUUCGGCGGACAUCCUU |
| | | AAUGAAGAACGGGGGGGUAACCCACCCCCCUGAUCUGCCC |
| 25 | Pelargonium | GCACAUGUAGUUCCCCUAACCUCUCAGUAGGAGUCAAGCCCUGAUGCGCCAACAUCC |
| | chlorotic ring | AGAAGGGGGUAAAGCUGAGUCGGUGCAAGGAACCUUGUUAGCC |
| | pattern virus | |
| | (PCRPV) | |
| 26 | Elderberry latent | AUAAUUAUAAUCAUGCAAUGGCACAUGUAGUUCCCCUAACCUCUCAGUAGGAGUCAAG |
| | virus (ELV) | CCCUGAUGCGCCAACAUCCAGAAGGGGGUAAAGCUGAGUCGGUGCAAGGAACCU |
| 27 | Rosa rugosa leaf | CCACGCGUAUGUACUAGAAUGUGAGUUGCUGGAGUCAGACCCUGGUGCGCCAACACC |
| | distortion virus | CAGAAGGGGGUCAACCAACUUGCUUUUAGUUACUGCGUGCGG |
| | (RrLDV) | |
| 28 | Pelargonium | CUUAUGUUUGCCGUAUGUGAAUGGUGGGAUGGGGGGGUUUUGGUAAUCAUUAGGGC |
| | ringspot virus | UACAGGACAACCGAGGAGGCGCCAACCUUCGACCAUCGGUGAUCCCCUAUUGCACUC |
| | (PelRSV) | CAGCCACCCAUUGUUACCUUCACUAGGCACCACUUAAG |
| 29 | Calibrachoa | CUAAGAGUUACUGCCACCAAAAGAGGAUAAUCAGACCACUUGUAGUUUGCUGCCACAA |
| | mottle virus | ACUGGUUGACAAGUUAAUGCCUCUCUCUUAGU |
| | (CbMV) | |

TABLE 4
PTE sequences

| SEQ ID | | |
|---|---|---|
| NO: | Virus | Sequence |
| 30 | Saguaro cactus | GCUUGACGUGGUUUGUUGCUCGACUGUGAGGGGACCUACCCACUGUGCUGCCACAC |
| | virus | AGGAACUUCCAACCUUCGGGUGGCGAGGUAGGGCAGAAGAGUGACUGAACGAGGAU |
| | (SCV) | GGUGUAGAGC |
| 31 | Pelarognium | GAGGGCAGUCACCCUACCUUCUGCCAGAGGUUCCCGGAGGAGUAGUGAUCCUUUGG |
| | flower break | GGGUGAUUCAUAGCUAGGAAAGGUCGGAGAGUUGGGAGGAGGCCAACUGGGGGAGG |
| | virus (PFBV) | UGCUAGGAGCACCAGAUGCAGCAGAGCAAAAAUCCGGGUUUCAUCGCCUGGUGCGGG |
| | | CGCUCAAUGCUGCUAACACGUUCCUGAAGGAAGAAAGGUUUCGGUCGAUGGAGACCU |
| | | UAACCCGCCC |
| 32 | Carnation mottle | AUGCAAUAGAAAAGUUUGUUAGACACACACGGCGGGACGUGUUGCCGGAGCUGCCAC |
| | virus (CarMV) | UCCGCCCAAGAUGGAUCGAUAUUAACCAUCUUUGGCAACAGCGUGCGUGGCUAAUCG |
| | | AACCUAGUAAUUGCAU |
| 33 | Honeysuckle | CCAACCACGACUGCCGUUAAAGAGCUUCAAUGGUAUGUCUCAAGACUAUAGACGGGA |
| | ringspot virus | UGAGCUUCUGGGGGCUGCCACCCUCGCCUCAUGCGCAUUGCCUGCUCUGGGCGCAU |
| | (HnRSV) | GUGGAAGCUGAUAUAGUUGAGAGCUUCGUGGAAGG |
| 34 | Pea stem | UAACACAGCAAUGGAGUAAGUGUACUCUGGUAGCAAUGGGUGGGGUCGAUUUCCUAC |
| | necrosis virus | GUCUAGCUUUGGCCGGCUAGCGUCUCUCGUAGUGGGGAAAUAACUCAUUGAGACCAG |
| | (PSNV) | CUCAGAACGCUGACAUUGUCUGUGUU |
| 35 | Hibiscus | GUGUGUAUUAUAUGGGCCUCCGUGCUGUUAGGGGCAGUGGAAACGUCGGUCUAGCC |
| | chlorotic ringspot | AACCGUCCCCUGGGUAGUGUGCUCCGUCUAAGUACACCACUACUCGGUUUCCACAAA |
| | virus (HCRSV) | CGAUCGGACUCCCAAAAUAGUGUACAUUAUGUUGAUUGCUUCUUUCCUCCUUUGUUA |
| | | UAUCUCCUCUCCCCUCUCCUCUACACUUGGAAUCCAUAAAAUCCUUCGGGUGGGCUU |
| | | GGAUUUCGGGUAC |
| 36 | Galinsoga | GUGAUCUUCUCUAGAGGAAGAUGCUUGGGGGUGUUCCCCUAAAGUUCAGAAUGGUCU |
| | mosaic virus | AUCAGGGAGCAGGCACUUUUCCGGCUUUGGCGAGUCGUCUCCACUUGUAUACGAACA |
| | (GaMV) | GUGAGGGGAAAAGUUAUCUGAUAGAAGAAUGAACCAUGGACAAGGACAC |
| 37 | Cocksfoot mild | ACCUGUUAAACAGACUCACCUCGGGCACGCGCUGAAAGUGCCCGUUGGAAGGAUUGC |
| | mosaic virus | GAUGGGCCAGUAUUCCUACUGUACCAGAGAACGUGUCAUCAGGGGAUGACCAUGUCC |
| | (CMMV) | ACCUUGCCGGGUGCCCUCGGACCUAUGUCCGUGACAUGGCGUGACGCUAGUAAUGG |
| | | UGCUAUGGAAGAAACACCUAACCC |
| 38 | Thin paspalum | CAGGCAGUCGGGUUGCAACUCGGCCUCCUACGCAUGAGCUAUGUCAUUGAGCUCAG |
| | asymptomatic | GGGAGCCACUGCAGGCAGUGGUGCCAGCGCAUAGACUCGCUCAGAGUCAUCCUAGG |
| | virus (TPAV) | CUAGGACCGGCCAGACAAAGGUCCGACACUACAUGCAGCGCUCUAGGUGUGUAGUGA |
| | | CAGUAAGCGCGCUCUAUCCGAAACUCCAGUGACAGGACAAGACCCCAGGCGGGACGC |
| | | CACACCCAUGUUUGCAGACAUGGCCCGAGGAAACUCUGGUGUGGGCUGGGGGGAAU |
| | | CCUAUGAUCACUGGACGGAAGGUAGAGAGUUCCAGCACAACCCCUUUAGGGUGGGCC |
| | | GUAAGUGCGUGGGAACAUUUCCUACAGUCUCACUGAGAAGAUUGGACGGGGUACCAU |
| | | GAGUACCCCUCACCAGGCCC |
| 39 | Japanese iris | AAACGACCAUUCUUGGGUUUGCAGCGAGAUGCCACGUGCCAGAGGAUAGUACCACUG |
| | necrotic ring | GCUGACCCUACCAGCGUUUGCGAGUUGGUAUGCGUGGAUGUAAAUUCCAAGUCUUGU |
| | virus (JINRV) | GUCCGACCUAGGCUCCGCCGGCUGGAGCUCGCAUGUGGCAGUGCGAGCAGAGGAUG |
| | | UCCCGAUGCAUAAAAUCCCGUGAGGGUGGGCUUGCUAGGGAGGCCGACACUGCGUA |
| | | AUGCAAAACUACCCUUUUCCCCUGUGAGGGGGAAGAGGGGGGCCC |
| 40 | PEMV2 | GUGAGCUCCCGCAGAGGGAUAAGCUGUCUCCCUGCGACGUAGUAGUAGAACACGUG |
| | | GGAUAGGGGAUGACCUUGUCGACCGUUUGUCGGUCCCCUGCUCCUUAGAGCUGGCA |
| | | AGGCGCCCAUUGGUUCUACAUUUCUACCAAAGUUGGUGGUGGAUGUCUCGCCCAAAA |
| | | AGAUCAUAAACGCGCGGGAUAAGGCCCUCUCCACCUU |
| 41 | Pea enation | GGUUCUAUCCGAACUACAGCACGGAAACACCUAAGACCCUCACAGACCUGAUGGACA |
| | mosaic virus 2 | GCGAAUCCCUCGUGUUAGUCCCUUCCCUCUCCGGGAAAACUUACCGCCCUAAGAUCG |
| | (PMV) | AAACACGAGGAAACCCACCGGAAUUGCGCAACAUAGAUGCCACCGCUUUCUCCGCUC |
| | | UCAGCGACGAAGAUAAGGGUGACUACUCGGUGGGAAGGCUGGUUGUUGGCUCCAGC |
| | | AAACAAGCUGUCGUGAUACAGCUCGGACUCCUCCGCAUGAGAUACUCAGCUGAAAUG |
| | | CGGGGAGCAACCUCCAUCAGUGGGGUUAGCGCAUAAACCUGCCCAAGGUUAUCACAG |
| | | GCUAGGACCGGCCUGAUAAAGGUCCGACUUGAUGUGCAGCUUCUUAGGUGCAUCUC |
| | | GUAUAGAAGCGCUCUGGACAUGACACCUUUGAGACAGACCGUACAGCAGUCACACGG |
| | | GACGCCACACCACCUUUGCAGAGGUGCCCUUGGGAAACCAAUGGUGUGGGGUGACAC |
| | | UGAUUAGUCGUUAACGGUGUACUAAUCCAGAGUAGUUCAGUGCAGGGGAAACCCGGG |
| | | CCGUAAGCACGUGGAACUAUAUAACUUAAUCUCGCUGAGAAGAUUGGGGGGGAUUCA |
| | | UGAAAUCCCCUCACCAGGCCC |
| 42 | Maize chlorotic | GGUGAGCCGGCAUGAGGUUGCAAGACCGGAACAACCAGUCCUUCUGGCAGAGUCCU |
| | mottle virus | GCCAAUCCAAAGUGUUUGGGAGCCUAAAUCGUAUACUAGUAGUUUGCGUGAUGACCA |
| | (MCMV) | UGACUGGAGAGUGGGCGGCGGCUGCCAACCGCAGACUGGGCGUAUAUAGUAAGCCU |
| | | UGACCCACCCAUGGACAACACGUACUUACGAACGGUGCGACAUGGUAACUGGAUACA |
| | | CUUAACCCUGGGGCAAGUAGAUGCUAGGAAACUAGCAUCGGGCCGCCCACGAGGGUU |
| | | UCUGAACUCAACGGAGUAGAACGGGGGGGGGGUAAUGCCCCUCUCUUCCGGCCC |
| 43 | Bermuda grass | GCUAGGAUGGCUAUCCUAUCUUAUUGGUGACAGAGUCACCCUGGGAAGCACACCCCU |
| | latent virus | UUAGUGCUCCCAGUAUGGGGGCUUGUCGGACCACCCCUCCAUUAGCGCUGUGGUGU |
| | (BGLV) | CUUGUCUCUUGCCUUUGCCGAGGCACAAAUGCUGCAUAGUAGCAGUGGGAGACAACA |
| | | GUACGCGACCGGGGAUAUAGAGUAGACUGACAAGUAGUCUGGGGAAAAACACAAGGG |
| | | AAACCUUGUGGGGCACCCGCGAGACUAUAGUAUCUCACCUGAGAUGAUAGGAGUUCC |
| | | CUCACGAGGGGAACCCACCUCCUGCCC |
| 44 | Adonis mosaic | AAGUAUAGAACGACACGUUAAACAGCUGAGCACAUGCCAGGGUGGUCUGGCGUCCCC |
| | virus (AdMV) | AACCCAUAAAGAUCCUAGAGGAAACUCUGGGUGAGGGCUUGGUAGUAGGGACAACCA |
| | | CCGUGUGAUUAAAAUCACAAAACACUAGCGGUUAGAGUUCAUAGCUCUUCUCGCGCC |
| | | C |
| 45 | Cucumber leaf | UGGCCUAGAUCGGAGGGGUCGUGGUAAUAGCACCGUUGGUGGUUCUUCGGAUGCUU |
| | spot virus | CUCAGUCCCAAGGCGCACAAGCAUGGGGGGGUUACCAUAACGAUCUAGGAGACAGCU |
| | (CLSV) | UAUCCGAGUACUCUGUGUCUGACUUGUAGAGAGACCACCAAUGCACACCAGAAAUGG |
| | | GGGCUAUAAGUGUAUUGGUUCUUAAUUUUCAUCCCUUGCGUGGAAACACGCUAGCCC |
| 46 | Pothos latent | GAUGUCGCUGGAAUCAUCCAAGGGCGGUGGCAUGAAGUCACAUCAGAAGAAAAAUAG |
| | virus | UAAGCCUAAUGGCCAUAGUAGGAGGGGUAAUCUAUCUGGGGAAGUUGGUGGUUCUU |
| | (PoLV) | CUUCCUCCUUGCCCUCAGGUGCACAGACUGGGGAGUGGAUAGAUAAUGACUAUGGAG |
| | | ACAGCUUAUCUGAGUAUUCUGGUGUGUCAGCAUAGCACACCCCAGGUGUCAUACCCU |
| | | GAAGUUUAGUAAGGGGGGCUAUAAAUGACGCUUGGUUUUACCUGAAAUCCCUUCCAU |
| | | GCUUUGCAUGGUAGCCC |

TABLE 5
ISS sequences

| SEQ ID | | |
|---|---|---|
| NO: | Virus | Sequence |
| 47 | Maize necrotic | GACAAACUCUCCGCCUAAUGUCUGAUGAUGUGAGGAACGUGGACUGUGAUGUGGUG |
| | streak virus | GUGCGGUACCAUGGCUGGUCACCAUGGUAAUGCGUAGGGCAACACAGUUCAUUAAGA |
| | (MNeSV) | CUCACUGAUGAUGGCACUAGGCACGGUUCACCCCCAUCCUUCGGGAGGGCUAUAGG |
| | | GGGUGACCGGGUUACACCACCGGAAGACCGGAACAUUGCCUUUGGGCAGCCC |
| 48 | Melon necrotic | AUCUCGCCUUAGGUUUACGGUUACCCGAGCUUCGCCUGAAACGGCGUUCCUCUAAUU |
| | spot virus | CCUUCCUGGUUCUUUCCCUCCACGGCUCGCGUGUUCUGCGGAACUGCAUCUGCUUC |
| | (MNSV264) | ACUUCCUUCUUUUUCCCUCUGUGGUGGUACGACCAAUGGGUGGCUCCCAUUGACUG |
| | | CGUAGGACAUGGAAAAAGAUUGAGUGGAGUGCACGAAGCGUCGCAAGAUCGCUGCCU |
| | | GACGAGGUCCAGCCAAUCAAAUACCAAACAUCUGGUUUGGGGGCGGAUUGGCUGUAU |
| | | GUCAUGUAAGUUUGAGGGGUUCUAUCGGAGCCCCCCGCCC |
| 49 | Cucumber | AAAGCGAACUUGACAGACUCUCAAGCAAGGUAUGAAGUUAAGGAGUGGAACCCGGAU |
| | Bulgarian virus | UAAUUGUAACCUGUAAUUAUUCUCGUGGUGGUACGACCUCAGGCUGGUCACCUGGGA |
| | (CBV) | CUGCGUAGGACGGGAGGUGUCCUCUUAUGGGCGCUAGUUAUUGGUGAAACGGGGAG |
| | | ACGGGUGGGGACGACACGGUUGUAUAUAGUCCACAACACCCACCUGUUGCAAACUCU |
| | | GGAAGAUAGUAUCUCGCACGGUUACACCCCAUCCUUCGGGAGGGCUUUAGGGGUGU |
| | | GCUGGAAGCACCACCGGACAGCCGGAACAUUGCCGAAAGGCAGCCC |
| 50 | Maize white line | CGUCCUGUCCGGUGGUAAGGGUCAAGCGACAGAAAAGAGGAUUUUGGCUGGUGGUG |
| | mosaic virus | GUACGGCCCGGGGAGUGGUUCCCCCGGGAUGCGUAGCGCCAGCUGAAGGAAUCCCA |
| | (MWLMV) | GUAAUCGCCACUAUAGAAGACCACUAGGACAGCAUGUACUCCACGCUUCGGCGGGGC |
| | | UAUAAGGAGUACAUGAUACCCCCCCCUAUCUUUCACCCAGCUUGCUGGGGUAGCCC |
| 51 | Johnsongrass | CGUCGUCGCUGUGUUGCCGAAGCAGAAAGCCCUAGGAAAGGGCCUAAAGCCUGUUG |
| | chlorotic stripe | GUGGUACGACUCCUGGUUUGGUCACCGGGAACUGCGUAGGAACAGACUGAAGGUCA |
| | mosaic virus | CUAGUACUGGCACUAUGGCGGCAUAAGCGACACACGGAGCCACACUUCGGUGGGGCU |
| | (JCSMV) | AUAAGGUUCCGUGUUGUAUCCCUCUUACUUUCA |
| 52 | Gompholobium | CUGUGCCAAACUUCCUUUGCAAGAUGCGACUCUCUUUGGGCCCACGUCAGGACUAGC |
| | virus (GoMVA) | CAUCCCAAGGACUAGUGGAAAGAGAGAAGUUGUUAUGCAGAGGAUAUGCGUCCACUA |
| | | UUGUGAAGACGUUAAAAGCAUACAGUCGCCAUCGCAAUGGUAGU |
| 53 | Cucumber | AAAGCGAACUUGACAGACUCUCAAGCAAGGUAUGAAGUUAAGGAGUGGAACCCGGAU |
| | Bulgarian latent | UAAUUGUAACCUGUAAUUAUUCUCGUGGUGGUACGACCUCAGGCUGGUCACCUGGGA |
| | virus | CUGCGUAGGACGGGAGGUGUCCUCUUAUGGGCGCUAGUUAUUGGUGAAACGGGGAG |
| | (CBLV) | ACGGGUGGGGACGACACGGUUGUAUAUAGUCCACAACACCCACCUGUUGCAAACUCU |
| | | GGAAGAUAGUAUCUCGCACGGUUACACCCCAUCCUUCGGGAGGGCUUUAGGGGUGU |
| | | GCUGGAAGCACCACCGGACAGCCGGAACAUUGCCGAAAGGCAGCCC |

TABLE 6
YSS sequences

| SEQ ID | | |
|---|---|---|
| NO: | Virus | Sequence |
| 54 | Tomato bushy | GACAGACUCUUCAGUCUGAGUUUGUGGAGAUGAGUGUAAAUCUGGCAUAGCAUACAG |
| | stunt virus | GUUACUCUUGUUGGGUUCUGGAUGUUAGGAUGACGAGUCGACUCGGGCUCCGCACU |
| | (TBSV) | AGGUUUGGUCGCCUAGGGGAUGGAGAUAUGGAAAGGGUCUCGUGUGGUAUCAGUCG |
| | | GUCGAAAGACGCGCUUCCAACAUGGGCCUAUGGUCGGAUAAGUCUUAGCAAUACCAG |
| | | CCAGCAUGAAUUGGAUUCCUGUUUACGAAAGUUAGGUGUCACUUGUGGAAGCGGACC |
| | | CAGACACGGUUGAUCUCACCCUUCGGGGGGCUAUAGAGAUCGCUGGAAGCACUACCG |
| | | GACAACCGGAACAUUGCAGAAAUGCAGCCC |
| 55 | Carnation Italian | GACAGACUCUUCGGUCUGAGGAUAGUGGAAGUGAGUGUGCCUAGUAUAGCAUGUAG |
| | ringspot virus | GUUACUCUCCUAGGAUUAUGUACUGUGUGUUAACGAGUCACGUCGGGCUCCGCGAU |
| | (CIRV) | GGGUUUGGUCGCCCAUGGGAUGGAGAUAUGGAAAGGGUCUCGCGCACUAUAAGUCU |
| | | AACUACGGUUGAGCUUGUGAUGUGGGCCUAUGCGUGGAUAAUACACACAUGUUGCAC |
| | | CCUAGGUGAAUUGGAUUCUUACUUACGAAUGCCAAGGACACUUGUGGAAGCUACUGA |
| | | CGACACGGUUGAUCUCACCCUUCGGGGGGGCUAUAGAGAUCGCUGGAAGCACUACC |
| | | GGACAACCGGAACAUUGCAGAAAUGCAGCCC |
| 56 | Cymbidium | GACAGACUCUUCAGUCUAGGUUGGAGGAAGCAGGAGAACACUGCUUGAGGGUACAGG |
| | ringspot virus | UUACUCUUAGUGAGACUAGGUUCGUGUGAUGACGAGUCAGGUCGGGCUCCGCGCUG |
| | (CymRSV) | GGUUUGGUCACCCAGGGGAUGGAGAUAUGGAAAGGAUCUCGUGCGCUAUAAGUCUAA |
| | | CGAAAGUUGAGCUUGCAACAUGGGUCUAUGCCUGAAUAAGUCACGUGAGUUGCCACC |
| | | ACUAUGAAUAGGAUUUUUGUUCACGACUAGCGGAGAACAUUUGUGGAAACCAACCAA |
| | | GACACGGUUGAUCUCACCCUUCGGGGGGGCUAUAGAGAUCGCUGGAAGCACUACCG |
| | | GACAACCGGAACAUUGCAGCAAUGCAGGGG |
| 57 | Cucumber | GACAGACUCUUCAGUCUGACUUGGUGGAAUCUUGCGAAUUUAACUGUUACUCUUCAU |
| | necrosis virus | GGGUUCCUUCCCAUACGAUGACGAGUCAGGUCGGGCCCUAUCUUAGGUUUGGUCAC |
| | (CNV) | CUAGGGGACGGGGAUAUGGAAAUCACUUUCGCUUGCUGUCAGUCUAGUGGAAACACU |
| | | UAGCUUGCAAUGUGGGUGUAUGCCUGGAUAAGUCGUAUGGAUGCUGGCCAUGAUGA |
| | | AUUGGAUGCAGUUUAGGACGUAUAGUGGAAAUCUUGCCAGACACGGUUGAUCUCACC |
| | | CUCCGGGGGGCUAUAGAGAUCGCUGGAACACUACCGGACAACCGGAACAUUGCAGAA |
| | | AUGCAGCC |
| 58 | Artichoke | GACAGACUCUUCAGUCUGAUUCUGUGGAAAUGAGAGUAAUUCUGACAUAGCAUACAG |
| | mottled crinkle | GUUACUCGUGUUGGGUUCUAGAUGUUAUGAUGACGAGCUGGUCGGGCUCCGCACGG |
| | virus (AMCV) | GUUUGGUCGCCCGGGGAUGGAGAUAUGGAAAGGGUCUCGUGUGCUGUUAGUCAGUC |
| | | GAAAGACGUGCUUGCAACAUGGGCCUAUAACCAGAUAAGUCAUAGCAAUACUAGCCAA |
| | | CAUGAAUUGGAUUCCUGUUUACGAAAGUUAGGUGUCACUUGUGGAGACAGAGUUAGA |
| | | CACGGUUGAUCUCACCCUUCGGGGGGGCUAUAGAGAUCACUGGAAGCACUACCGGAC |
| | | AACCGGAACAUUGCAGCAAUGCAGCCC |
| 59 | Pelargonium | GACAGACUCUUCAGUCUGAUGUUACGGAGAUGAGUAGAACAAAGUGAGGGACUAUGG |
| | necrotic spot | GUUACCCUUUGUAGAUUUGGUCCGCAUGAUGACGAGUUAGGUCAGGCCCCGCGCCA |
| | virus (PNSV) | GGUUUGGUCACCUGGGGGAUGGGGACACGGAAAGGAUCUCGCAUGCUGUUAGUCUG |
| | | ACGAAAGUCGAGCUUGCAAUGUGGGUCUAUGCCUAAAUAAGUCGUGUGGGUUGCCGC |
| | | CUACAAGGAAUUGGAUGCCACUUUUCUCUCAGUGAGCUCUCUCAUUGAAGUAGCAUU |
| | | AGACACGGUUGAUCUCACCCUUCGGGGGGGCUAUAGAGAUCGCUGGAAGCACUACCG |
| | | GACAAACCGGAACAUUGCAGAAAUUCAGCCC |
| 60 | Grapevine | GACAGACUCUUCAGUCUGAGUUGGUGGAUGUGAGUGUACUUUGUUUGAGCAUACAG |
| | Algerian latent | GUUACUCUUAGUAUGAUUAGGUCUACGUGAUGACGAGUCAGGUCAGGCUCCGCACUG |
| | virus (GALV) | GGUUUGGUCGCCCAGGGGAUGGAGACACGGAAAGGACAUCGUGUGCUAUAAGUCUG |
| | | ACGUAAGUCGAGCUUGCAACAUGGGUCUAUGCCUGGAUAAGUCACGUGGUUGCCCCU |
| | | ACUAUGAAUUGGAUGCCUGUUUACGACUAGCAAACGACACUCGUGGAAACUAAUUUG |
| | | GACACGGUUGAUCUCACCCUUCGGGGGGGCUAUAGAGAUCGCUGGAAGCACUACCG |
| | | GACAACCGGAACAUUGCAGAAAUGCAGCCC |
| 61 | Pelargonium line | GACAGACUCUUCAGUCUGAGUUUGCUGAAAUGAGUGUAAAUCUGGCAUAGCAUACAG |
| | pattern virus | GUUACUCUCGUUGGGUUCUAGAUGUCAUGAUGACGAGUCGGUCGGGCUCCGCACUA |
| | (PLCV) | GGUUUGGUCGCCUAGGGGAUGGAGAUAUGGAAAGGGUCUCGUGUGCUGUUAGUCAG |
| | | UCGGAAGACGUGCUUGCAACAUGGGCCUAUAACCGGAUAAGUCAUGACAAUACUAGC |
| | | CAACGUGAAUUGGAUUCCUGUUUACGAAAGUUAGGUGUCACUUGUGGAAACAAACCU |
| | | AGACACGGUUGAUCUCACCCUUCGGGGGGGCUAUAGAGAUCGCUGGAAGCACUACCG |
| | | GACAACCGGAACAUUGCAGAAAUGCAGCCC |
| 62 | Pear latent virus | GACAGACUCUUCACGCUUGGAGUGAAGUUUGGUGGCAACUAUACACCUUAGACUACU |
| | (PeLV) | GGUUACUACUGUCAGGUUGGAACCCACAUUAAUUGAGCUAGGUCGGGCCCCGUCGUA |
| | | GGAUUGGUCACCUACGGAAUGGGGAUAUGGAAAGGAUCUCGUACCCUGUGAGUCAAA |
| | | CGAAAGUUGUGCUUGGGAUAUGGAUCUAUGCCUAGUAGGUUAUGUGGUGUGUCUCU |
| | | GACUUUAAUUGGAUGCCAGUUUUCAACAGGAGUGGAAGUAACUGAACAACACUUUGA |
| | | GCACGGUUGAUCUCACCCUCCGGGGGGGCUAUAGAGAUCGCUGGAAACACUACCGGA |
| | | CAACCGGAACAUUGCAGAAAUGCAGCCC |
| 63 | Lisianthus | GACAGACUCUUCACGCUUGGAGUGAAGUUUGGUGGCAGCUAUACACCUUAGACCACU |
| | necrosis virus | GGUUACUACUGUCAGGUUGGAACCCACACUAAUUGAGCUAGGCCGGGCCCCGUCGUA |
| | (LNV) | GGUUUGGUCACCUACGGAAUGGGGAUAUGGAAAGGAUCUCGUACCCUGUGAGUCAAA |
| | | CGAAAGUUGUGCUUGGGAUAUGGGUCUAUGCCUAGUAGGUUAUGUGGUGUGUCUCU |
| | | GACUUUAAUUGGAUGCCAGUUUUCAACAGGAGUGGCAGCGACUGAACAACACUUUGA |
| | | GCACGGUUGAUCUCAUCCUUCGGGGGGGCUAUAGAGAUCACCGGAAGCACUACCGGA |
| | | CAACCGGAACAUUGCAGAAAUGCAGCCC |

TABLE 7
TSS sequences

| SEQ ID | | |
|---|---|---|
| NO: | Virus | Sequence |
| 64 | Turnip yellow | GUUCUCGAUCUUUAAAAUCGUUAGCUCGCCAGUUAGCGAGGUCUGUCCCCACACGAC |
| | mosaic virus | AGAUAAUCGGGUGCAACUCCCGCCCCUCUUCCGAGGGUCAUCGGAACC |
| | (TYMV) | |
| 65 | Red clover | UUGGUUCUUUUAAGUGUAGCCUCCACCCGAGUUGCAAGAGGGGAACACGCAGUCUCG |
| | necrotic mosaic | CCGACCCUGUUGGCAAACAGUAAAAUUGCAAAAAAUAGAGUGCUAGGAGUAGUUCCC |
| | virus (RCNM) | GUACCCGCGGGAGCAAGACCCUACUACAGUAGACGAACCGGCAUCGGACCCUGGGAA |
| | | ACAGGUACCUAGCGUAUUAAUAGGUCGCUAGAAAUCGGGCGGGCUUCACCUUCGGGU |
| | | AACCCGCCGUCGAGAGGGAAAACCUCUGUGUGGUUGUGCGCACGUCGUUGUCGGAA |
| | | GACUCUCACUGUUGUAGUUAUUUCCUUUUUCUUUUAGGUAGGAGCACUUGCUCAGAG |
| | | GGCGCAUACCUCUGGUAAAACAAAAUUGGCCCCUGAGAGUAGAAAACUCAGGUUAAUA |
| | | AAACAGGAGAAUAUUUACCUAGUCGUAUAACGGCUAGGUACCCC |
| 66 | Turnip crinkle | AACGGUGGCAGCACUGUCUAGCUGCGGGCAUUAGACUGGAAAACUAGUGCUCUUUGG |
| | virus | GUAACCACUAAAAUCCCGAAAGGGUGGGCUGUGGUGACCUUCCGAACUAAAAGAUAG |
| | (TCV) | CCUCCCUCCUCGCGCGGGGGGGGGGGCCUGCCC |
| 67 | Cardamine | GGUGUGGAAACCAGAGAGUCUUGCGGUAACCAACGAAAUCUCUUCGGAGUGGGUGUU |
| | chlorotic fleck | GGGGACCACCAAAUGAAGAAAAUAGCCCCUCCCUCGCGCAGGGGGGGGCCAGCCC |
| | virus (CCFV) | |

TABLE 8
Additional translation enhancer elements

| SEQ ID | | |
|---|---|---|
| NO: | Description | Sequence |
| 68 | Cucurbit | CCGCUCUGUGGAGACGAGCGUGACUCCACCCGGCAUCCAGUGGGCCCGACCAAAUCAC |
| | aphid-borne | UGAUGACAUCAAGCCAAAGAUGUAAAAUUGGAACGACUCCGAAAGGAUAGGCAACGAAC |
| | yellow virus | GUUCCCACCUUAGUGGAAACAGGGGGACUCCCCCUGGCAUUUCGGUGU |
| | (CABYV-X) | |
| 69 | Citrus yellow | AUAGGGUCAUUGGUUUACCGAUGAUACCUGUUCAGAAUAGGAUUGCUCGAGCUUCGUU |
| | vein- | GGUUAGGGUAACUCACAUACCUUCUUCCAUAACUGGAAAAGGUCGUGUGAGCAACCUAA |
| | associated | CCAGUUAAUGUAGGUGUCUUUCCGUAUCUAGUCACGAUGGUAAGCAACCCGUUUAUCU |
| | virus (CYVaV) | GUACGGCGCUCACCCGUGGGUAGGAAGGUGAAGGUUUUGUGUCCUUUAGGUCUUGGA |
| | | CAGUCUGCGGGCUUGGGAACGACGCCCCGCUAGCAACGUACUGCUCUCCUACCGGACU |
| | | GGUAGCUUAAUUGUCAUCUUGGAGCGAUAGCACUGUGGGCCUCACCCUUCGCGCGUUG |
| | | GACGUGUUGCGUGCCCCCCACAGAUUUGUGAAACUCUAUGGAGCAGUUCCGCGAGCCA |
| | | GAAGGGAGGAUGGCCGCCUGGCGUAAUCCAGGAGCUCUGGGGGGCUUGUACUCAGAG |
| | | UAGCAUUCUGCUUUAGACUGUUAACUUUAUGAACCACGCGUGUCACGUGGGGAGAGUU |
| | | AACAGCGCCC |
| 70 | Opuntia | GGGUAAUUAUGGAUUUAAAUGAAACCCAUGGUUACUCGU |
| | umbra-like | |
| | virus (OULV) | |
| 71 | Fig umbra-like | AAAUAUGGAUUCGAUAUCAAUGCCCAAAGCCUGU |
| | virus P1 | |
| | (FULV2) | |
| 72 | Histone4E | GCUGAAGGUGUUCCUGGAGAACGUGAUCCGCGACGCCGUCACCUACACGGAGCACGCC |
| | | AAGCGCAAGACCGUCACCGCCAUGGACGUGGUCUACGCGCUCAAGCGCCAGGGCCGCA |
| | | CUCUCUACGGAUUCGGCGGUUAAUCGACUAACAAACGAUUUUC |
| 73 | scrameIF4g10x | AAAAAAGUCAGUACCAUCACGGAAUUAUUCUCGCUUAUGGCCUGAUAAAAAAGUCAGUA |
| | | CCAUCACGGAAUUAUUCUCGCUUAUGGCCUGAUAAAAAAGUCAGUACCAUCACGGAUUT |
| | | AUUCUCGCUUAUGGCCUGAUAAAAAAGUCAGUACCAUCACGGAAUUAUUCUCGCUUAUG |
| | | GCCUGAUAAAAAAUUCAGUACCAUCACGGAAUUAUUCUCGCUUAUGGCCUGAUAAAAAA |
| | | GUCAGUACCAUCACGGAAUUAUUCUCGCUUAUGGCCUGAUAAAAAAGUCAGUACCAUCA |
| | | CGGAAUUAUUCUCGCUUAUGGCCUGAUAAAAAAGUCAGUACCAUCACGGAAUUAUUCUC |
| | | GCUUAUGGCCUGAUAAAAAAGUCAGUACCAUCACGGAAUUAUUCUCGCUUAUGGCCUGA |
| | | UAAAAAAGUCAGUACCATCACGGAAUUAUUCUCGCUUAUGGCCUGAU |
| 74 | mineIF4gapt10x | AAAAAAACUCACUAUUUGUUUUCGCGCCCAGUUGCAAAAAGUGUCGAAAAAAACUCACUA |
| | | UUUGUUUUCGCGCCCAGUUGCAAAAAGUGUCGAAAAAAACUCACUAUUUGUUUUCGCGC |
| | | CCAGUUGCAAAAAGUGUCGAAAAAAACUCACUAUUUGUUUUCGCGCCCAGUUGCAAAAA |
| | | GUGUCGAAAAAAACUCACUAUUUGUUUUCGCGCCCAGUUGCAAAAAGUGUCGAAAAAAA |
| | | CUCACUAUUUGUUUUCGCGCCCAGUUGCAAAAAGUGUCGAAAAAAACUCACUAUUUGUU |
| | | UUCGCGCCCAGUUGCAAAAAGUGUCGAAAAAAACUCACUAUUUGUUUUCGCGCCCAGUU |
| | | GCAAAAAGUGUCGAAAAAAACUCACUAUUUGUUUUCGCGCCCAGUUGCAAAAAGUGUCG |
| | | AAAAAAACUCACUAUUUGUUUUCGCGCCCAGUUGCAAAAAGUGUCG |
| 75 | TBR1 | CACACAAAGCAACAAAAGAACGAAGCAACAAAAGCAUAGAUUUAUUGAAAUGAAAGUACA |
| | | UUCUACAGAGUGGGGGCAGGCU |
| 76 | TBR2 | GAGAAAUGCAAAUCAAAACCACAAUGGAAUACCAUCUCACGCCAGUCAGAAUGGCAAUUA |
| | | UUAAAAAAUCACAACAAUUAAUGAUGGCAAGGCUGUG |
| 77 | TBR3 | AACAAUCAAUGGAAGCAGAAACAAAUAAACCAAGGUGUGCAUCAAGGAAUACAUUCACGC |
| | | AUGAUGGCUGUAUGAGUAAAA |
| 78 | TBR4 | GCUCAAGGAAAUAAAAUAGGACACAAAGAAAUGGAAAAACAUUCCAUACUCAUGGAUAGA |
| | | AAGAAUCAAUAUCAUGAAAUGGCC |
| 79 | TBR5 | GCAAUAACCAAACAACCUCAUUAAAAAGUAGGCAAAGGACAUAAACAGACACUUUUCAAA |
| | | AGAAGACAUACACGUGGCCAACAAACAUAU |
| 80 | TBR6 | GACUUAAACAUAAGACCUAAAACCAUAAAAACCACAGAAGAAAACAUAGGCAAUGCCAUU |
| | | CAGGACAUAGGCAUGGGCAAAGACUUC |
| 81 | TBR7 | UACAAAGAACUCAAACAAAUCAGCAAGAACAAAAACAAUCCCAACAAAAUGUUGGACAAA |
| | | GACAUGAAUAGACAAUUCUCGAAAGAAGAUGUACAAAUGGCU |
| 82 | TBR8 | GACAGUCAGACAAUCACAAAGAAACAAGAAUGAAAAUGAAUGAACAAAACCUUCAAGAAA |
| | | UAUGGGAUUAUGAAGAGGCCAAAUGU |
| 83 | TBR9 | GGACAAAUUGCUAGAAAUAAACAAAUUACCAAAAAUGAUUCAAGUAGAGACAGAGAAUCA |
| | | AAAUAGAACUACACAUAAGUGGGCC |
| 84 | TBR10 | GGAAAUAACAGAGAACACAAACAAAUGGGAAAACAUUCCAUGUUCAUGGAUAGGAAGAAU |
| | | CAAUAUUGUGAAAAUGGCCAUACU |
| 85 | TBR11 | GCAAAGAAAGAAUGAAGCAGCAAAAGAACGAAAGCAGGAAUUUAUUGAAAACCAAAGUAC |
| | | ACUCCACAGUAUGGGAGCGGACCCG |
| 86 | TBR12 | GGAAUUUAAGCAAAUCAACAAGCAAAACCAAAAUAAUCCCAUUAAAAAGUGGGUAAAGGA |
| | | CAUGAAUACACACUUGUCAAUAGAGGACAUUCAAGUGGCCAAC |
| 87 | TBR13 | UCAGUCUAGAUCUUAAAGGAACACCAGAGGGAGUAUUUAAAUGUGCCCAAUAAGCAAGA |
| | | AUUAUGGUGAUGUGGAAGU |
| 88 | TBR14 | CAGACCAUAAAUAAACACAGAAGACACACGAGUGUAAAGUCAGUGCCCCGCUGCGAAUU |
| | | AAAUCGGGGUGAUGUGAUGGCGAGUGAGUGGGUAGUU |
| 89 | TBR15 | GAAAUGGAAUCGAAGAGAAUGGAAACAAAUGGAAUGGAAUUGAAUGGAAUGGAAUUGAA |
| | | UGGAAUGGGAAC |
| 90 | TBR16 | AGGAAAUACAAGACAACAAACACAGAAACACAACCAUCGGGCAUCAUGAAACCUCGUGAA |
| | | GAUAAUCAUCAGGGU |
| 91 | TBR17 | GAAAGGAUUCGAAUGGAAUGAAAAAGAAUUGAAUGGAAUAGAACAGAAUGGAAUCAAAU |
| | | CGAAUGAAAUGGAAUGGAAUAGAAAGGAAUGGAAU |
| 92 | TBR18 | GAAAGAAUCAAGAGGAAAUGCAAGAAAUCCAAAACACUGUAACAGAUAUGAUGAAUAAUG |
| | | AGGUAUGCACUCAUCAGCAGACUCGACAU |
| 93 | TBR19 | UCAAAAGAAAAGCAACCUAACAAAUACGGGAAGAAUAUUUGAAUAGACAUUUCACAGGAA |
| | | AAGAUAUAUGAAUGGCCAAAAAGCAAAUG |
| 94 | TBR20 | CAGUUCAAAAAUUAUUGCAACAAAAUGAGAGAGAUGAGUUUAUCUUGCAAACUAAUGGA |
| | | UGGUAGCAGUGACAGUGGCAAAACGUGGUUUGAUUCU |
| 95 | TBR21 | GUAAUGGAAUGGAAUGGAAAGGAAUCGAAACGAAAGGAAUGGAGACAGAUGGAAUGGAA |
| | | UGGAACAG |
| 96 | TBR22 | UAUACGCAAAUCAAUAAAUGUAAUCCAGCAUAUAAACAGUACUAAAGACAAAAACCACAU |
| | | GAUUAUCUCAAUAGAUGCAGAAAAGGCC |
| 97 | TBR23 | GACCGAAACAACAACAGAAACAGAAACAAACAACAAUAAGAAAAAAUGUUAAGCAAAACAA |
| | | AUGAUUGCACAACUUACAUGAUUACUGAGUGUUCUAAUGGU |
| 98 | TBR24 | CCACUGCUCAAGGAAAUAAGAGAGAACACAAACAAAUGAAAAAACAUUCCAUGCUCAUGG |
| | | AUAGGAAGAAUC |
| 99 | TBR25 | GCAAUCGAAUGGAAUGGAAUCGAACGGAAUGGAAUAAAAUGGAAGAAAACUGGCAAGAA |
| | | AUGGAAUC |
| 100 | TBR26 | GAAAUCAUCAUCAAACGGAAUCGAAUGGAAUCAUUGAAUGGAAUGGAAUGGAAUCAUCA |
| | | UGGAAUGGAAAC |
| 101 | TBR27 | UGUUGAGAGAAAUUAAACAAAGCACAGAUAAAUGGAAAAACGUGUUCAUAGAUUGAAAGA |
| | | CUUCAUGUUGUAUGGUGUC |
| 102 | TBR28 | CCAACAUAAGACAAAGAAACAUCCAGCAGCUGCCUAUGGCAAAAGAUUACAAUGUGUCAA |
| | | ACAAGAGGGCAAU |
| 103 | TBR29 | CCCAACUUCAAAUUAUACUACAAGGCUACAGUAAUCAAAAAAGCAUAGUACUAUUACAAA |
| | | AACAGACACACAGGCCAAUGGAAUACAAU |
| 104 | EMCVUTR | UAGUGUAGUCACUGGCACAACGCGUUACCCGGUAAGCCAAUCGGGUAUACACGGUCGU |
| | | CAUACUGCAGACAGGGUUCUUCUACUUUGCAAGAUAGUCUAGAGUAGUAAAAUAAAUAG |
| | | AUAGAG |
| 105 | CVB3UTR | AUUAGAGACAAUUUGAAAUAAUUUAGAUUGGCUUAACCCUACUGUGCUAACCGAACCAG |
| | | AUAACGGUACAGUAGGGGUAAAUUCUCCGCAUUCGGUGCG |
| 106 | HCV1b | ACGGGGGGCUAGUCACUCCAGGCCAAUAGGCCAUUCUGUUUUUUUUUUUUUUUUUUUU |
| | | UUUUUUUUUUUUUUUUUUUUUUUUUUUUUUUUUUUUUUUUUUUUUUUCCUUUUUCUUC |
| | | UUCCUUUUCUUCUUUCUUUGGUGGCUCCAUCUUAGCCCUAGUCACGGCUAGCUGUGAA |
| | | AGGUCCGUGAGCCGCAUGACUGCAGAGAGUGCUGAUACUGGCCUCUCUGCAGAUCAUG |
| | | U |
| 107 | HCV2a | AGCGGCACACACUAGGUACACUCCAUAGCUAACUGUUCCUUUUUUUUUUUUUUUUUUUU |
| | | UUUUUUUUUUUUUUUUUUUUUUUCUUUUUUUUUUUUUUCCCUCUUUCUUCCCUUCUCA |
| | | UCUUAUUCUACUUUCUUUCUUGGUGGCUCCAUCUUAGCCCUAGUCACGGCUAGCUGUG |
| | | AAAGGUCCGUGAGCCGCAUGACUGCAGAGAGUGCCGUAACUGGUCUCUCUGCAGAUCA |
| | | UGU |
| 108 | RotaVP63UTR | GGACCAAGCUAACAACUUGGUAUCCAACUUUGGUGAGUAUGUAGCUAUAUCAAGCUGUU |
| | | UGAACUCUGUAAGUAAGGAUGCGUAUACGCAUUCGCUACACAGAGUAAUCACUCAGAUG |
| | | GUAUAGUGAGAGGAUGUGACC |
| 109 | BMV | UGCCCCUGCUCGGAGAGCCCCUGACUGGGUUAAAGUCACAGGCCCCUUGUCUCAGGUA |
| | | GAGACCCUGUCCAGGUAGGACACUUUGGCUAAGGUUAAAAGCUUGUUGAAUCAGUACAA |
| | | UAACUGAUAGUCGUGGUUGACACGCAGACCUCUUACAAGAGUGUCUAGGUGCCUUUGA |
| | | GAGUUACUCUUUGCUCUCUUCGGAAGAACCCUUAGGGGUUCGUGCAUGGGCUUGCAUA |
| | | GCAAGUCUUAGAAUGCGGGUACCGUACAGUGUUGAAAAACACUGUAAAUCUCUAAAAGA |
| | | GACCAGCAA |
| 110 | HBVPRE | UAAUCAACCUCUGGAUUACAAAAUUUGUGAAAGAUUGACUGGUAUUCUUAACUAUGUUG |
| | | CUCCUUUUACGCUAUGUGGAUACGCUGCUUUAAUGCCUUUGUAUCAUGCUAUUGCUUC |
| | | CCGUAUGGCUUUCAUUUUCUCCUCCUUGUAUAAAUCCUGGUUGCUGUCUCUUUAUGAG |
| | | GAGUUGUGGCCCGUUGUCAGGCAACGUGGCGUGGUGUGCACUGUGUUUGCUGACGCA |
| | | ACCCCCACUGGUUGGGGCAUUGCCACCACCUGUCAGCUCCUUUCCGGGACUUUCGCUU |
| | | UCCCCCUCCCUAUUGCCACGGCGGAACUCAUCGCCGCCUGCCUUGCCCGCUGCUGGAC |
| | | AGGGGCUCGGCUGUUGGGCACUGACAAUUCCGUGGUGUUGUCGGGGAAGCUGACGUC |
| | | CUUUCCAUGGCUGCUCGCCUGUGUUGCCACCUGGAUUCUGCGCGGGACGUCCUUCUG |
| | | CUACGUCCCUUCGGCCCUCAAUCCAGCGGACCUUCCUUCCCGCGGCCUGCUGCCGGCU |
| | | CUGCGGCCUCUUCCGCGUCUUCGCCUUCGCCCUCAGACGAGUCGGAUCUCCCUUUGG |
| | | GCCGCCUCCCCGCCUGUUUCGCCUC |
| 111 | WPRE | AAUCAACCUCUGGAUUACAAAAUUUGUGAAAGAUUGACUGGUAUUCUUAACUAUGUUGC |
| | | UCCUUUUACGCUAUGUGGAUACGCUGCUUUAAUGCCUUUGUAUCAUGCUAUUGCUUCC |
| | | CGUAUGGCUUUCAUUUUCUCCUCCUUGUAUAAAUCCUGGUUGCUGUCUCUUUAUGAGG |
| | | AGUUGUGGCCCGUUGUCAGGCAACGUGGCGUGGUGUGCACUGUGUUUGCUGACGCAA |
| | | CCCCCACUGGUUGGGGCAUUGCCACCACCUGUCAGCUCCUUUCCGGGACUUUCGCUUU |
| | | CCCCCUCCCUAUUGCCACGGCGGAACUCAUCGCCGCCUGCCUUGCCCGCUGCUGGACA |
| | | GGGGCUCGGCUGUUGGGCACUGACAAUUCCGUGGUGUUGUCGGGGAAGCUGACGUCC |
| | | UUUCCAUGGCUGCUCGCCUGUGUUGCCACCUGGAUUCUGCGCGGGACGUCCUUCUGC |
| | | UACGUCCCUUCGGCCCUCAAUCCAGCGGACCUUCCUUCCCGCGGCCUGCUGCCGGCUC |
| | | UGCGGCCUCUUCCGCGUCUUCGCCUUCGCCCUCAGACGAGUCGGAUCUCCCUUUGGG |
| | | CCGCCUCCCCGCCUG |
| 112 | BYDV 5′ UTR | GAGUGAAGAUUGACCAUCUCACAAAAGCUGUUACGUGCUUGUAACACACUACGCGCCCG |
| | | UUUUGUAUUCGGGAAGUAGUUGCGAAAACGGUCCCCUUAUUGCCUGACAAGCUAAGGG |
| | | CCACCCUUCUUUCCCCACCGCCAU |
| 113 | BYDV 3′ UTR | GUCUUAGCAAGCUCUGAGCCAGGAGAUGGACAUAAACCAUAGCAAUCCAACGUGUAACC |
| | | GCAAUGGGGCAAACAACAGGUGAACCGUGUCCACGGGCCUGGUUACCGAAAGGAAAGC |
| | | CAGUAUCCAACACAGCAAUGUGUUGGGGGUCACACCUUCGGGGUACUCUUAACGCUGA |
| | | CACUCGAAAGAGCAGUUCGGCAACCC |
| 114 | TNV-D 5′ UTR | GAUACCUUACCAGUAUCUCAGUGAUUAAGUAAUCAGCU |
| 115 | TNV-D 3′ UTR | UCGCUUUCAUAGAUCCGUCUUCCCAGAGACGUUAAGAAGAAGCUGGAGAAAAAUAUUAG |
| | | GUUAGAAGCUUGGGCGUGACAAACCCAAGUUGCAUCUCUUACGUGGUUAAUCACACUG |
| | | UAUGUUGACGAAUAGGCCGGAUCCUGGGAAACAGGUUUAACGGGCUCUCUGUGGUGGA |
| | | GGGCCGACGCAUCACCUAUUUGUGCUCCAACAGUGGUUGUCAUCACGUGUCCUGACAU |
| | | GGCUCCAUGCGACAGCAUGGGGGGGUCCAGAGUCAGUCCCCUCUUUAUUUACCUAGGA |
| | | UUUCCUAGGAACCC |
| 116 | TCV 5′ UTR | GGUAAUCUGCAAAUCCCUGGCACCCGCCUAAAAUUGCCCUCAUCAACCUUCUCUCUAUU |
| | | CACG |
| 117 | TCV 3′ UTR | CGGUGGCAGCACUGUCUAGCUGCGGGCAUUAGACUGGAAAACUAGUGCUCUUUGGGUA |
| | | ACCACUAAAAUCCCGAAAGGGUGGGCUGUGGUGACCUUCCGAACUAAAAGAUAGCCUCC |
| | | CUCCUCGCGCGGGGGGGGGGGCCUGCCC |
| 118 | Dengue | GUUGUUAGUCUACGUGGACCGACAAAGACAGAUUCUUUGAGGGAGCUAAGCUCAACGU |
| | | AGUUCUAACAGUUUUUUAAUUAGAGAGCAGAUCUCUGCACA |
| 119 | WNV 5′ UTR | GUAGUUCGCCUGUGUGAGCUGACAAACUUAGUAGUGUUUGUGAGGAUUAACAACAAUU |
| | | AACACAGUGCGAGCUGUUUCUUGGCACGAAGAUCUCG |
| 120 | WNV 3′ UTR | GAUAGUAUUAUAGUUAGUUUAGUGUAAAUAGGAUUUAUUGAGAAUGGAAGUCAGGCCAG |
| | | AUUAAUGCUGCCACCGGAAGUUGAGUAGACGGUGCUGCCUGCGGCUCAACCCCAGGAG |
| | | GACUGGGUGACCAAAGCUGCGAGGUGAUCCACGUAAGCCCUCAGAACCGUCUCGGAAG |
| | | GAGGACCCCACGUGCUUUAGCCUCAAAGCCCAGUGUCAGACCACACUUUAAUGUGCCAC |
| | | UCUGCGGAGAGUGCAGUCUGCGAUAGUGCCCCAGGUGGACUGGGUUAACAAAGGCAAA |
| | | ACAUCGCCCCACGCGGCCAUAACCCUGGCUAUGGUGUUAACCAGGGAGAAGGGACUAG |
| | | AGGUUAGAGGAGACCCCGCGUAAAAAAGUGCACGGCCCAACUUGGCUGAAGCUGUAAG |
| | | CCAAGGGAAGGACUAGAGGUUAGAGGAGACCCCGUGCCAAAAACACCAAAAGAAACAGC |
| | | AUAUUGACACCUGGGAUAGACUAGGGGAUCUUCUGCUCUGCACAACCAGCCACACGGC |
| | | ACAGUGCGCCGACAUAGGUGGCUGGUGGUGCUAGAACACAGGAUCU |
| 121 | SDV 5′ UTR | GUAAAGUUGACACCUUUACAGAAGUGAUCUUGCUUGUAAGAGUUAAUUCAUCAAGAGUU |
| | | AAUACAAGAUCCACCUCCCGGCACCUUCGUAUCGUGUUUGAGGUAUCUCUAGUGUUUG |
| | | GUUGUUUGAAUACUAGGUAAUACUUA |
| 122 | SDV 3′ UTR | UAUAGUAGAAGUAAGUGUGCGCUUUCCAUCACAUUGAAUUGGAGCCAACCUUGGUGAG |
| | | UUGCUGUCGUGGGAUCCUGGGAAACAGGUUCGGUGAACAAAAAGCCGGUAAUCUCGGG |
| | | CGUUCGUCAGACGCCGUCGUAUCAACACAGUAUGCAUAUCCAUCAAUAACCCAAUUUUC |
| | | UUAGUGACUAAAAAUUAGUGUUAGUUUUUCCGCCUCUCUUGUAAACCACAUACAAGAUG |
| | | CGAGCUUGUGGAGAGUACCAUCCCUUGAAUAAGGAUGGUUGAAAGUUUCUGGUCCCAA |
| | | UCCAUUGAUCAAAAUUGGCACUGCUUCUGGUGACUACACUGCCGCUGGCGAUACAGCG |
| | | UAAACAAUUUCUACUUCUUGUUCGAUGAAACGAGAACAAUUAGUAAAGUCUUGCCGUUC |
| | | ACAUGUCUAUCUGACGGUACUAGGCUUCGUCCCUCUGAUGACGUUAAAUGAAAGCGGA |
| | | GUAAACCGACAUCACCUUACUAGGUUGGGUUAACCUAGGGAAACCGGGAAAAUGCCCG |
| | | GGACACUUGGUUUAGUGGAUUUUAAACUCGAGGGCUCUUCCUCGUUAAAAAGGGGGUA |
| | | GCCAGGUUGAAAACUGGCACUAGGGAGCGAUCCCUCUUUGUUGUUAAGGUGGCUUUGU |
| | | GUCCACCUGCCCCC |
| 123 | TYMV 5′ UTR | GUAAUCAACUACCAAUUCCAGCUCUCUUUUGACAACUGGUCUUAUACCCACUUCCGUAC |
| | | ACUUGCAACCCUCGUAAGACAAUUGCAAAUGAGU |
| 124 | TYMV 3′ UTR | GUUCUCGAUCUUUAAAAUCGUUAGCUCGCCAGUUAGCGAGGUCUGUCCCCACACGACA |
| | | GAUAAUCGGGUGCAACUCCCGCCCCUCUUCCGAGGGUCAUCGGAACC |
| 125 | Zika 5′ UTR | GUUGUUGAUCUGUGUGAAUCAGACUGCGACAGUUCGAGUUUGAAGCGAAAGCUAGCAA |
| | | CAGUAUCAACAGGUUUUAUUUUGGAUUUGGAAACGAGAGUUUCUGGU |
| 126 | Zika 3′ UTR | GCACCAAUCUUAGUGUUGUCAGGCCUGCUAGUCAGCCACAGCUUGGGGAAAGCUGUGC |
| | | AGCCUGUGACCCCCCCAGGAGAAGCUGGGAAACCAAGCCCAUAGUCAGGCCGAGAACG |
| | | CCAUGGCACGGAAGAAGCCAUGCUGCCUGUGAGCCCCUCAGAGGACACUGAGUCAAAA |
| | | AACCCCACGCGCUUGGAGGCGCAGGAUGGGAAAAGAAGGUGGCGACCUUCCCCACCCU |
| | | UUAAUCUGGGGCCUGAACUGGAGAUCAGCUGUGGAUCUCCAGAAGAGGGACUAGUGGU |
| | | UAGAGGAGACCCCCCGGAAAACGCAAAACAGCAUAUUGACGCUGGGAAAGACCAGAGAC |
| | | UCCAUGAGUUUCCACCACGCUGGCCGCCAGGCACAGAUCGCCGAAUAGCGGCGGCCGG |
| | | UGUGGGGAAAUCCAUGGGUCU |
| 127 | Lobtrop | CCAAGGCAGGCCCACGGUCCUAGCGUUCUCAGUUCUUUUUUGUUGGUUGGGAAACUCC |
| | | UAAAAAACCGCCAC |
| 128 | TMV 5′ UTR | GUAUUUUUACAACAAUUACCAACAACAACAAACAACAAACAACAUUACAAUUACUAUUUAC |
| | | AAUUA |
| 129 | TMV 3′ UTR | GUCCUGCAACUUGAGGUAGUCAAGAUGCAUAAUAAAUAACGGAUUGUGUCCGUAAUCAC |
| | | ACGUGGUGCGUACGAUAACGCAUAGUGUUUUUCCCUCCACUUAAAUCGAAGGGUUGUG |
| | | UCUUGGAUCGCGCGGGUCAAAUGUAUAUGGUUCAUAUACAUCCGCAGGCACGUAAUAAA |
| | | GCGAGGGGUUCGAAUCCCCCCGUUACCCCCGGUAGGGGCCC |
| 130 | PEMV2 | GGGUAUUUAUAGAGAUCAGUAUGAACUGUGUCGCUAGGAUCAAGCGGUGGUUCACACC |
| | | UGACUUCACCCCUGGCGAGGGCGUGAAGUCU |
| 131 | STNV 5′ UTR | AAAAAAAGUAAAGACAGGAAACUUUACUGACUAA |
| 132 | STNV 3′ UTR | UAAUCCCAGAGGUUCACAAUGUUAGUGAUGGGCGCUGAAAGAUGCGUAGCUACCCUUC |
| | | UGGAGCCACUUCCUGGUGGUAAGCAGAAAUCCAAGGGUACGGUGGUACGGUGGAAAGC |
| | | AGUCCCAGCUCUGCAUUGGGAACCGGCUCACACCCAGCUUAGGGCUAAAGUGUACUAC |
| | | UUGCUCAUUUGUAGUCUAAAUGAGACGUUGGCCUCGCCGUGUCGAGGUGGCCUAAGGG |
| | | AUUGGAACCCCUGAUGGUCGUAGUCGAAUUUCCCGUGUUUCAUCCCGAGUCUCUUGGU |
| | | CAUAAUGCCAUUAGUAGGUCAAGCACCCAACGUAACUUCAAAGAUAUCCUCCUUGCAAC |
| | | AAGAAUAUGUGCGCUGUCUGUGUUUAAAGCGGUAUAUUUUGGGCGCCGGCAUAUCGUU |
| | | GCUUGGACCAGGGCCCCACGCCGGUUGGUACCCGGGUGGCUUCCCCUCGUUCACAGG |
| | | GCUUUAGGAGAUGAUAAGGUAUAGUUACUAGGCAAAUGCGGACAAACCUGAAAAGCUCG |
| | | CUAGUGGUGGGCUGGCCAAGCGAAGAACCUCAUCCAGGUAUAGUUCUACAUGGGAAAU |
| | | UUGGUACCAUCCAAACUUCUAUGAAGUCCUCGACUAACCCC |
| 133 | BRV | UAGCGAUGUGCACGUUUAGGAGAUAAAUAGCUAGUAUCUUUCUACUCGGAUUCCCCUC |
| | | UGUCGUCAUAGAGGGGUUUCAAGGGAUGUCAUCAUCAGCGUUGGCUAU |
| 134 | YFV5UTR | GUAAAUCCUGUGUGCUAAUUGAGGUGCAUUGGUCUGCAAAUCGAGUUGCUAGGCAAUA |
| | | AACACAUUUGGAUUAAUUUUAAUCGUUCGUUGAGCGAUUAGCAGAGAACUGACCAGAA |
| 135 | YFV3UTR | CACCAUCUAACAGGAAUAACCGGGAUACAAACCACGGGUGGAGAACCGGACUCCCCACA |
| | | ACCUGAAACCGGGAUAUAAACCACGGCUGGAGAACCGGACUCCGCACUUAAAAUGAAAC |
| | | AGAAACCGGGAUAAAAACUACGGAUGGAGAACCGGACUCCACACAUUGAGACAGAAGAA |
| | | GUUGUCAGCCCAGAACUCCACACGAGUUUUGCCACUGCUAAGCUGUGAGGCAGUGCAG |
| | | GCUGGGACAGCCGACCUCCAGGUUGCGAAAAACCUGGUUUCUGGGACCUCCCACCCCA |
| | | GAGUAAAAAGAACGGAGCCUCCGCUACCACCCUCCCACGUGGUGGUAGAAAGACGGGG |
| | | UCUAGAGGUUAGAGGAGACCCUCCAGGGAACAAAUAGUGGGACCAUAUUGACGCCAGG |
| | | GAAAGACCGGAGUGGUUCUCUGCUUUUCCUCCAGAGGUCUGUGAGCACAGUUUGCUCA |
| | | AGAAUAAGCAGACCUUUGGAUGACAAACACAAAACCACU |
| 136 | Boone 3′ UTR | AUGGAUUGGAUUUCGGUUGAGCCCCCACCCGGUACAACGCUUUACCUUAGAAGCCACU |
| | | AAGGUGUACGCGGUCAUCGGGGACCCCUCCUGGCCUUUGGUUUAUUGGUGAAUUACUA |
| | | GUUCAGUUAGGUUUUGUUAGUUAGGAAAAAAAAAAAAAAAAAAAAAAAAAA |
| 137 | Aichivirus 3′ | UAGUUAGGGAACACAGUGCCUCGGCACGCUUAGUUAGUUAGAACCCUUUGCCCACUCC |
| | UTR | UCCAUGGUGAUAUAAAGACCACCCACUUCCUUCGGGUGAGCCCCUAAGCCAUGGUUGU |
| | | ACUGCACUAUCAUCCUAAGACGGUCCUUCUUCGGAUCGCAAUCUCACCCUGGUGCCGC |
| | | GCUUCCUUCGGGAACUGCACCCGCGGACCAGGGCCGUCUUUGAACUUUUCUAACUGUU |
| | | CUUAC |
| 138 | Saffold virus | ACAUCCUCUCGAUCGGAUCGCAACGUGUUACCCAGGAAUCCACUUGGGUGUACGCGGC |
| | 3′ UTR | CGUUCUGACGUUGGAAUUCUGUAGAUGAAAGUUAGCUAGGAGCUUUUAAUUGGAAAUG |
| | | AGAACAAA |
| 139 | CYBA 3′ UTR | CCUCGCCCCGGACCUGCCCUCCCGCCAGGUGCACCCACCUGCAAUAAAUGCAGCGAAG |
| | | CCGGGA |
| 140 | CYBA 3′ UTR | CCUCGCCCCGGACCUGCCCUCCCGCCAGGUGCACCCACCUGCAAUAAAUGCAGCGAAG |
| | v2 | CCGGGACCUCGCCCCGGACCUGCCCUCCCGCCAGGUGCACCCACCUGCAAUAAAUGCA |
| | | GCGAAGCCGGGA |
| 141 | Rabbit | GGGGCGCGUGCGCACCCGCGUGUGAACAGUCGCCCUUCCGCCACCUGGGCGUGGCCU |
| | picornavirus 3′ | CCAUCAGAGCUACUCUUCCUGAGUGCUUUCUUUUUCCUCUUUUCUUUUCUUUCUUCUU |
| | UTR | UUCUUUUCUGUUUCUGUGUGUAGAGAGUGAAUGCAGCA |
| 142 | Aichivirus B 3′ | CCUCAGAACUCUGGCCUCCCACUGUUCGUCCUGGUAGAAUAGACAAGCUUUUCACUAUC |
| | UTR | CCUAAGACCACUCUCUCCGUGGCAUUUUCCUCUCAGUGUCCUGCUCCCUCACGGGACU |
| | | GCACCCAGGAACUGUGAGCUUCCCCCCCUGUCCUUGAAAUUUCUAUUCUUUUCUUGC |
| 143 | Porcine | GACACCAGAUACACUCUUGCUCUGUAUCGUCCCUGUGUAGAAUAGACUCAUGCUUUCCU |
| | kobuvirus 3′ | AAGACCGGAUCUCUCUUCCGGCACUCUCACCCGCCUGCCCUGCCUUCAUCUUGAAGCU |
| | UTR | GCACCCAGGGAGGCGGUCCCUGACUGUCCUUUACUUUCUAUUCUGUAUUGCAA |
| 144 | Aichivirus D 3′ | AGAGACAGGUCUCCUGUGUCCCUCUGCGUUCUCUCUGGUUUCCUGUAAGUUAGGCUCU |
| | UTR | UUGUAAUGGUGUUGGUCUCUAUUGCAUAAAAUUCUUCCUCCUUUGCUCUCCCUCCACC |
| | | UCUCGGGGGGGGGGGCCCGCCCUUUUCUUCCUAACUUGAAAAAAAAAAAAAAAAAAAAA |
| | | AAA |
| 145 | AMV (Alfalfa | AUUAGUCAUUGGUAAUUCAAUGCCAACCUCCACUGGGUGGGUUAAGGUUGAGGUAUAG |
| | mosaic virus) | AAUCCUAUUCGCUCCUGAUAGGAGAAAUUCUAUAUUGCUUAUAUACGUGCUUAUGCACG |
| | 3′ UTR | UAUAUAAAUGCUCAUGCUAAAUUGCAUGAAUGCCCCUAAGGGAUGC |
| 146 | AIMP1 | AAUAAACAAUAAACCAAUAAACCAAUAAACCCAAUAAACAAUAAACC |
| 273 | AIMP1 | AATAAACAATAAACCAATAAACCAATAAACCCAATAAACAATAAACC |
| 147 | IFIT2 | AGUGUAAAGUGUAAAAGUGUAAAAGUGUAAAAAGUGUAAAGUGUAA |
| 274 | IFIT2 | AGTGTAAAGTGTAAAAGTGTAAAAGTGTAAAAAGTGTAAAGTGTAA |
| 148 | CLK3 | UCGAUGAUCGAUGAAUCGAUGAUCGAUGAAAUCGAUGAUCGAUGA |
| 275 | CLK3 | TCGATGATCGATGAATCGATGATCGATGAAATCGATGATCGATGA |
| 149 | MTDH | UUGCCGAUUGCCGAAUUGCCGAUUGCCGAAAUUGCCGAUUGCCGA |
| 276 | MTDH | TTGCCGATTGCCGAATTGCCGATTGCCGAAATTGCCGATTGCCGA |
| 150 | UBAP2L | AUGCCGAAUGCCGAAAUGCCGAAAUGCCGAAAAUGCCGAAUGCCGA |
| 277 | UBAP2L | ATGCCGAATGCCGAAATGCCGAAATGCCGAAAATGCCGAATGCCGA |
| 278 | EJC2 | CGGCGCTCGGCGCTTCGGCGCTTTCGGCGCTTTTCGGCGCTT |
| 151 | EJC3 | CGCCGCUCGCCGCUUCGCCGCUUUCGCCGCUUUUCGCCGCUU |
| 279 | EJC3 | CGCCGCTCGCCGCTTCGCCGCTTTCGCCGCTTTTCGCCGCTT |
| 152 | EJC4 | CGGGGCUCGGGGCUUCGGGGCUUUCGGGGCUUUUCGGGGCUU |
| 280 | EJC4 | CGGGGCTCGGGGCTTCGGGGCTTTCGGGGCTTTTCGGGGCTT |
| 153 | EJC5 | GCCGCCGCCGCUUUGCCGCCGCCGCUUUUGCCGCCGCCGCUU |
| 281 | EJC5 | GCCGCCGCCGCTTTGCCGCCGCCGCTTTTGCCGCCGCCGCTT |
| 154 | EJC6 | GGCGGCCGCCUUUGGCGGCCGCCUUUUGGCGGCCGCCUUUUU |
| 282 | EJC6 | GGCGGCCGCCTTTGGCGGCCGCCTTTTGGCGGCCGCCTTTTT |
| 155 | EJC7 | CCCCAGCACCUUUCCCCAGCACCUUUUCCCCAGCACCUUUUU |
| 283 | EJC7 | CCCCAGCACCTTTCCCCAGCACCTTTTCCCCAGCACCTTTTT |
| 156 | EJC8 | GAAGAAGAAGUGAAGAAGAAGUUGAAGAAGAAGUUUGAAGAA |
| 284 | EJC8 | GAAGAAGAAGUGAAGAAGAAGUUGAAGAAGAAGUUUGAAGAA |
| 157 | EJC9 | CCAGCCGCACCCAGCCGCACCCAGCCGCACCCAGCCGCACCC |
| 285 | EJC9 | CCAGCCGCACCCAGCCGCACCCAGCCGCACCCAGCCGCACCC |
| 158 | EJC7-8 | CCCCAGCACCUUUCCCCAGCACCGAAGAAGAAGUUUGAAGAA |
| 286 | EJC7-8 | CCCCAGCACCTTTCCCCAGCACCGAAGAAGAAGTTTGAAGAA |
| 159 | EJC8-7 | GAAGAAGAAGUGAAGAAGAAGUUCCCCAGCACCUUUCCCCAGCAC |
| 287 | EJC8-7 | GAAGAAGAAGUGAAGAAGAAGTTCCCCAGCACCTTTCCCCAGCAC |

The expression augmenting element may include a translation enhancer fused to a spacer element. The spacer element may be conjugated to the 5′ end of the translation enhancer. In some embodiments, the spacer element may be conjugated to the 3′ end of the translation enhancer.

### Stability Elements

The polyribonucleotides described herein may include a first expression augmenting element wherein the first expression augmenting element may include a stability element. The second expression augmenting element may include a stability element. The second spacer element may include an expression augmenting element. One or more of the expression augmenting elements may be a stability element. In some embodiments, the stability element is a translation enhancer. In some embodiments, a stability element is a spacer element.

The stability element may be a 5′ UTR or a 3′ UTR. In some embodiments, the 5′ UTR or 3′ UTR is a human UTR. In some embodiments, the 5′ UTR or 3′ UTR comprises any one of the 5′ UTRs or 3′ UTRs described in Table 9 below, or a portion thereof. The 5′ UTR or 3′ UTR may be from a gene encoding a TRAM1, a TMED2, a VAMP3, CRIP, an AP2A2, a PSMD5, a GPX4, or a PRKAB1. The 5′ UTR or 3′ UTR may be a 5′ UTR 3′ UTR from a human beta actin, DDB2, TP5313, FcIgG, LSP1, AES, DRB4, or a mitochondrially encoded 12S rRNA.

The stability element may include between 50 and 2000 ribonucleotides (e.g., between 50 and 1500, 50 and 1000, 50 and 500, 50 and 100, 100 and 2000, 500 and 2000, 1000 and 2000, or 1500 and 2000). The stability element may include between 20 and 750 ribonucleotides (e.g., between 20 and 700, 20 and 650, 20 and 600, 20 and 550, 20 and 500, 20 and 450, 20 and 400, 20 and 350, 20 and 300, 20 and 250, 20 and 200, 20 and 150, 20 and 100, 20 and 50, 50 and 100, 50 and 750, 100 and 750, 150 and 750, 200 and 750, 250 and 750, 300 and 750, 350 and 750, 400 and 750, 450 and 750, 500 and 750, 550 and 750, 600 and 750, 650 and 750, and 700 and 750 nucleotides). For example, the stability element may have a length of between 100 and 500 ribonucleotides (e.g., between 100 and 450, 100 and 400, 100 and 350, 100 and 300, 100 and 250, 100 and 200, 100 and 150, 150 and 500, 200 and 500, 250 and 500, 300 and 500, 350 and 500, 400 and 500, or 450 and 500 ribonucleotides).

The expression augmenting element may include a stability element fused to a spacer element. The spacer element may be conjugated to the 5′ end of the stability element. In some embodiments, the spacer element not encoding an expression augmenting element may be conjugated to the 3′ end of the stability element.

The stability element may be any known stability element. In some embodiments, the stability element has a nucleic acid sequence having at least 85% (e.g., at least 85%, 86%, 87%, 88%, 89%, 90%, 91%, 92%, 93%, 94%, 95%, 96%, 97%, 98%, 99%, or 100%) sequence identity to any one of the sequences described in TABLE 9. In some embodiments, the stability element has a nucleic acid sequence having at least 90% (e.g., at least 90%, 91%, 92%, 93%, 94%, 95%, 96%, 97%, 98%, 99%, or 100%) sequence identity to any one of the sequences described in TABLE 9. In some embodiments, the stability element has a nucleic acid sequence having at least 95% (e.g., at least 95%, 96%, 97%, 98%, 99%, or 100%) sequence identity to any one of the sequences described in TABLE 9. In some embodiments, the stability element has a nucleic acid sequence of any one of the sequences described in TABLE 9.

TABLE 9
Stability Element Sequences

| SEQ ID | | |
|---|---|---|
| NO: | Name | Sequence |
| 160 | TKrandom | CGCAUAUAUCGAUCAGCGCAGGUAGAGAAGCUACAUCACCAGCAUGCGAGAGA |
| 161 | CHrandom | GGAGUGCUAGAGGAUGACCUUAAAUCAGGGACGAUAUUAUAAUUGACUAA |
| 162 | Alpha | GUUCCUCCUGCCCGCUGGGCCUCCCAACGGGCCCUCCUCCCCUCCUUGCACCGG |
| 163 | TRAM1 | UGAAUUAUAAACUAAUUGAUUAAUGUCCCCAAAGAAAUCUGCUUUCUACUAUAUCU |
| | | UUCAGCAUUAGAGAUUUUUCUGUUCUUGAAAAUACAGUCUGUGCUCUUUGAUUUU |
| | | UGCUAUUGUACGGUUUCAUGCAUUUUUUUAAAGGGCAUUUGAGGGGAGGAUUAU |
| | | UGCUAUGAAUGAAAAAAAUAUUUUAGCUUAGACUA |
| 164 | TMED2 | AAAGCCUCUUCCUGAUGAUCCCAACUCAGAAUUCACUGUUUACCAAACACCUUGG |
| | | UCAUAAUAAUGUCAUUAGUUUCUCCAUUUUUAUUUUCUGAACUGUACAUUCACAA |
| | | CUUAUGUUUCUUUGAGAUUAAUAGAUAUUGGGGGAAAAACGCCUUUUUAGGAAAA |
| | | UUAUAGUGAAAAUUUGACAGUUGAUUGGCAUAAUUUCUUGUUUGAAUGCUGCCUC |
| | | CAUUAUAUAGGUCCUUCCAGGAACUCAAACACUGUAAGUGAAAUAUGGGAGUAUA |
| | | GUUUUUAUUAUUUCUUCUUUUCCUUUUGUUUUCAUAAUAUAAUGCAGUUUGUUCA |
| | | GGAAAUCAGCACAAAGCCUGAUAGUACUUUACUA |
| 165 | VAMP3 | AGAACCAGCGGAACUCAAAACUGCUGUUCAAGAAACCUCUUCAAGACUUUUGACU |
| | | UAGAACCUGCUAUAUUAUCAAGCUUACCUACUGUUAUCUCUAAAAUUUUUUUUGU |
| | | GUUAAUGUAAAGUUGAAUUUCUAGGAAACGUGCCUUUGUUUUUUAAUAUGCACUC |
| | | CAAAUUAGAAGGCCGGCCCCGUCCACAUUUUGC |
| 166 | CRIP | AUGUAUUGAUGGAAUUUCUGGCUUUCUAAAUGAUUUUACUUUCUGCCUUGAAUUU |
| | | UCAAGGCAUAGAUGUCAACUUACAGAAUAACAUGUUUUAAGAUAAUUAAGUUUAAA |
| | | CCAGAGAAUUUGAUUGUUACUCAUUUUGCUCUCAUGUUCUAAACAGCAACAGUGU |
| | | AACUAGUCUUUUGUUGUAAAUGGUUAUUUUCCUU |
| 167 | AP2A2 | AAACAAUGGUGUGUUGUUUGAAAACCAGCUGCUUCAAAUUGGACUUAAGUCUGAA |
| | | UUUCGGCAGAAUUUAGGUCGGAUGUUUAUCUUUUAUGGUAAUAAGACCUCCACGC |
| | | AGUUCCUAAACUUUACCCCAACACUAAUCUGUUCAGACGACCUUCAGCCUAACCU |
| | | GAACCUGCAGACCAAGCCCGUGGACCCGACCGUGGAGGGGGGCGCGCAGGUGCA |
| | | GCAGGUGGUCAACAUAGAGUGCGUGUCCGACUUCACGGAGGCGCCAGUCCUCAA |
| | | CAUUCAGUUCAGGUAUGGGGGCACCUUCCAGAACGUGUCUGUGCAGCUGCCCAU |
| | | CACUCUCAACAAAUUCUUCCAGCCGACAGAAAUGGCUUCUCAGGAUUUCUUUCAA |
| | | CGUUGGAAGCAGUUGAGCAAUCCACAGCAGGAAGUGCAGAACAUCUUCAAAGCAA |
| | | AGCACCCAAUGGA |
| 168 | PSMD5 | UUUCUUCUAGAGCUCAUGUAGAGGACCACGUUUUGACCAAAACUUCUCCUAAGGC |
| | | AUUUGACUCCAUCUAUAUUUCACAAAAGAGACUUCCUUUCCCCAAGAAUUAUCAU |
| | | GGAAUGUCAGAUGUUACUUUGUUACCAACACUGUUAUAUUUCUACAUUGAAAUGC |
| | | AAAGUGGAACUAGGAGUUUGGAAUGCAUUAAGAGCAGACAAGCUUGGUCAUAAUA |
| | | GAUCCAGUGUUUUUCAGAUUCCUUUCACUGCCUUAAUCUUUGCAACAGGGUGGAA |
| | | GUUUUUUUCUUCCCUCAAAAUUUUCAUGGACAUGCAAUCUUAUCUAAAAGCCUGC |
| | | UUCAGGGCUGGGCGCGGUGGCUAACACCUAUAAUUCCCAGCACUUUGGGAGGCC |
| | | AAGAUGGGCAAAUCACUUGAGUCUAGGAGUUCAAGACCAGCCUGGCCAAUAUGGC |
| | | AAAACCCUGUCUCUACUAAAAAUAUAAAAUCAGCCAGGCAUGGUGGCGCACACCU |
| | | AUAAUC |
| 169 | PRKAB1 | AGAGCUGGGGGGGGAUGGUGGCCCAGGAGACAGCACACCACCAGGCUCCACACG |
| | | UGCAUGCUUUCCCCAAGAGGGAAUGGACUGUACAUUGCUCAUUUCACACUCUUCA |
| | | GAAGACAUUUCAUACCUGCCCUGGUCCUGCUUGAAGGUUUGUCCAGGCAGAGCA |
| | | GCUCCUGCAGCGCCUCGGUCUGUGACAGUCCUCCUAGCACCCCCAUGGCUUUGA |
| | | GCCUCGGGGACUCAUCAAGUCCAAGAAAAGAGGGAGGGGUGGCAGAGGAUCUGC |
| | | AGCCCUGGCCCCGCGGUGCAUGAGGCUGGGUGCAGUUCUAAACCUACAUUCUCG |
| | | AUUUUUCUUAAGCCAAAAAUGAAUGCUAACUCCUUUGCCAGUAAAAUUCUGGGAA |
| | | ACAGGGACUGAGGCCACACAUCAUUUCCAGUCAUCUGUGUGUUUUUAAGGCCAG |
| | | CCACUUGUCCCUGUUGAGGCCUGGCUAUGGAACUAAAUACAGUGUUGGUCUUGC |
| | | CUGUCCUUCAAA |
| 170 | Ad2VAI | GGGCACUCUUCCGUGGUCUGGUGGAUAAAUUCGCAAGGGUAUCAUGGCGGACGA |
| | | CCGGGGUUCGAACCCCGGAUCCGGCCGUCCGCCGUGAUCCAUGCGGUUACCGCC |
| | | CGCGUGUCGAACCCAGGUGUGCGACGUCAGACAACGGGGGAGCGCUCCUUUU |
| 171 | DDB2 | GACACUAAAGAAGGUGUGGGCCAGACAAGGCCUUGGAGCCCACACAUGGGAUCAA |
| | | GUCCUGCAAGCAGAGGUGGCGAUUUGUUAAAGGGCCAAAAGUAUCCAAGGUUAG |
| | | GGUUGGAGCAGGGGUGCUGGGACCUGGGGCACUGUGGGACUGGGACACUUUUA |
| | | UGUUAAUGCUCUGGACUUGCCUCCAGAGACUGCUCCAGAGUUGGUGACACAGCU |
| | | GUCCCAAGGGCCCCUCUGUAUCUAGCCUGGAACCAAGGUUAUCUUGGAACUAAAU |
| | | GACUUUUCUCCUCUCAGUGGGUGGUAGCAGAGGGAUCAAGCAGUUAUUUGAUUU |
| | | GUGCUCACUUUUGAUAUGGCCAAUAAAACCAUACCGACUGA |
| 172 | TP5313 | GGAGGAUGGGGCAGGACAGGACGCGGCCACCCCAGGCCUUUCCAGAGCAAACCU |
| | | GGAGAAGAUUCACAAUAGACAGGCCAAGAAACCCGGUGCUUCCUCCAGAGCCGUU |
| | | UAAAGCUGAUAUGAGGAAAUAAAGAGUGAACUGGAA |
| 173 | HBA13UTR | CUCGAGCUGGUACUGCAUGCACGCAAUGCUAGCUGCCCCUUUCCCGUCCUGGGU |
| | | ACCCCGAGUCUCCCCCGACCUCGGGUCCCAGGUAUGCUCCCACCUCCACCUGCC |
| | | CCACUCACCACCUCUGCUAGUUCCAGACACCUCCCAAGCACGCAGCAAUGCAGCU |
| | | CAAAACGCUUAGCCUAGCCACACCCCCACGGGAAACAGCAGUGAUUAACCUUUAG |
| | | CAAUAAACGAAAGUUUAACUAAGCUAUACUAACCCCAGGGUUGGUCAAUUUCGUG |
| | | CCAGCCACACCCUGGAGCUA |
| 174 | FclgG | GGCCAGGCCCCUUCCCUCUGCCCCCGGGUGCUUGAAGUCUAGCCCCAUCCUGGU |
| | | CCAAUGCGCUCUUGGUAGCCUCCUUUCCCAGCUGCCCGCCCGCCGCCAUGCCGC |
| | | CCUUACUGCCCCUGCGCCUGUGCCGGCUGUGGCCCCGCAACCCUCCCUCCCGGC |
| | | UCCUCGGA |
| 175 | LSP1 | UUCCAGCCAGACACCCGCCCCCCGGCCCUGGCUAAGAAGUUGCUUCCUGUUGCC |
| | | AGCAUGACCUACCCUCGCCUCUUUGAUGCCAUCCGCUGCCACCUCCUUUUGCUC |
| | | CUGGACCCUUUAGCCUCUCUGCCCUUCCACUCUCUGACCCC |
| 176 | AES | CUGGUACUGCAUGCACGCAAUGCUAGCUGCCCCUUUCCCGUCCUGGGUACCCCG |
| | | AGUCUCCCCCGACCUCGGGUCCCAGGUAUGCUCCCACCUCCACCUGCCCCACUC |
| | | ACCACCUCUGCUAGUUCCAGACACCUCC |
| 177 | DRB4 | CUUUGCAGGAUGAAACACUUCCCCGCUUGGCUCUCAUUCUUCCACAAGAGAGACC |
| | | UUUCUCCGGACCUGGUUGCUACUGGUUCAGCAACUCUGCAGAAAAUGUCCUCCC |
| | | CUGUGGCUGCCUCAGCUCAUGCCCUUUGGCCUGAAGUCCCAGCAUUGAUGGCAG |
| | | CCCCUCAUCUUCCAAGUUUUGUGCUCCCCUUUACCUAACGCUUCCUGCCUCCCAU |
| | | GCAUCUGUACUCCUCC |
| 178 | 12S | CAAGCACGCAGCAAUGCAGCUCAAAACGCUUAGCCUAGCCACACCCCCACGGGAA |
| | | ACAGCAGUGAUUAACCUUUAGCAAUAAACGAAAGUUUAACUAAGCUAUACUAACCC |
| | | CAGGGUUGGUCAAUUUCGUGCCAGCCACACC |
| 179 | RnaseLInh | AAAAUUUAAAAAAAACUGAGACAAAUGAUGGAGUCUUGAUCGUGAACACUAGCAAG |
| | | UACCCCAAUAUGUAUGUUCCUGUCGGUGCUGUGACUGAACAGGGAUAUCUAAAUC |
| | | UCGGUGGGCGCCAAACUGCUCGUACUCUAAUGUACAACUUUCCAACCAGAGCAGG |
| | | ACAGUGUGGUGGAGUCAUCACAUGUACUGGGAAAGUCAUCGGGAUGCAUGUUGG |
| | | UGGGAACGGUUCACACGGGUUUGCAGCGGCCCUGAAGCGAUCAUACUUCACUCA |
| | | GAGUCAAGGUGAAAUCCAGUGGAUGAGACCUUCGAAGGAAGUGGAUUAAAAAAAA |
| | | AUAAAAAA |
| 180 | RNaseLinhdel | AAAUUUAAAAAAAACUGAGACAAAUGAUGGAGUCUUGAUCGUGAACACUAGCAAGU |
| | | ACCCCAAUAUGUAUGUUCCUGUCGGUGCUGUGACUGAACAGGGAUAUCUAAAUCU |
| | | CGGUGAAAACAUCGGGAUGCAUGUUGGUGGGAACGGUUCACACGGGUUUGCAGC |
| | | GGCCCUGAAGCGAUCAUACUUCACUCAGAGUCAAGGUGAAAUCCAGUGGAUGAGA |
| | | CCUUCGAAGGAAGUGGAUUAAAAAAAAAUAAAAAA |
| 181 | CN43 5′ UTR | GCGUGAGGAAAGUACCAAACAGCAGCGGAGUUUUAAACUUUAAAUAGAC |
| | | AGGUCUGAGUGCCUGAACUUGCCUUUUCAUUUUACUUCAUCCUCCAAG |
| | | GAGUUCAAUCACUUGGCGUGACUUCACUACUUUUAAGCAAAAGAGUGGU |
| | | GCCCAGGCAAC |
| 182 | CN43 3′ UTR | AAUUCAGACAAGGCCCACAGAAUAAGAUUUUCCAUGCAUUUGCAAAUAC |
| | | GUAUAUUCUUUUUCCAUCCACUUGCACAAUAUCAUUACCAUCACUUUUU |
| | | CAUCAUUCCUCAGCUACUACUCACAUUCAUUUAAUGGUUUCUGUAAACA |
| | | UUUUUAAGACAGUUGGGAUGUCACUUAACAUUUUUUUUUUUUGAGCUAA |
| | | AGUCAGGGAAUCAAGCCAUGCUUAAUAUUUAACAAUCACUUAUAUGUGU |
| | | GUCGAAGAGUUUGUUUUGUUUGUCAUGUAUUGGUACAAGCAGAUACAG |
| | | UAUAAACUCACAAACACAGAUUUGAAAAUAAUGCACAUAUGGUG |
| 183 | HCK | UUCGGGCAAGGCAUGCGAGAUAAAAAGGGUCUCGUAUGAGGGCGUGGC |
| | | AACCCCUCCCCCCCAGCUGCGGCGGCACAAAAGCGUCUCGCGUGUCGU |
| | | CG |
| 265 | BPgV | ACUUGGCAAGGCACCCCCUGGCUAGUGGCUGCACUUAACUGAGUGGCC |
| | | GGCUAGUCGGUGGGUGGGGGCACGUGACUCCCCCUUCCCCACACCGG |
| 266 | TABV | GGCAAGGUACGGAUUAGCCGUAGGGGCUUGAGAACCCCCCCUCCCCAC |
| | | UC |
| 290 | ACADVL | ATACTCCCGGCCAGGGCCTGTCCCAGTTATGTGCCTTCCCTCAAGCCAAA |
| | | GCCGAAGCCCCTTTCCTTAAGGCCCTGGTTTGTCCCGAAGGGGCCTAGTG |
| | | TTCCCAGCACTGTGCCTGCTCTCAAGAGCACTTACTGCCTCGCAAATAATA |
| | | AAAATTTCTAGCCAGTCA |
| 291 | ADAM15 | CCTCTCCGGAGGTTCCGCTGCCTCCAAGCCGGACTTAGGGCTTCAAGAG |
| | | GCGGGCGTGCCCTCTGGAGTCCCCTACCATGACTGAAGGCGCCAGAGAC |
| | | TGGCGGTGTCTTAAGACTCCGGGCACCGCCACGCGCTGTCAAGCAACAC |
| | | TCTGCGGACCTGCCGGCGTAGTTGCAGCGGGGGCTTGGGGAGGGGCTG |
| | | GGGGTTGGACGGGATTGAGGAAGGTCCGCACAGCCTGTCTCTGCTCAGT |
| | | TGCAATAAACGT |
| 292 | ATP50 | AAGTGTTGGTTTTCTGCCATCAGTGAAAATTCTTAAACTTGGAGCAACAAT |
| | | AAAAAGCTTCCAGAACAGATCA |
| 293 | C3 | CCACACCCCCATTCCCCCACTCCAGATAAAGCTTCAGTTATATCTCACGTG |
| | | TCTGGAGTTCTTTGCCAAGAGGGAGAGGCTGAAATCCCCAGCCGCCTCAC |
| | | CTGCAGCTCAGCTCCATCCTACTTGAAACCTCACCTGTTCCCACCGCATTT |
| | | TCTCCTGGCGTTCGCCTGCTAGTGTG |
| 294 | CAPG | GGGTGGGCGTCTTCCTGCCCCATGCTCCCCTGCCCCCCACCACCTGCCT |
| | | GCTTGCTTCTCTGGCTGCCTGGTCAGTGCAGAGGTGCCCCCTGCAGATGT |
| | | TCAATAAAGGAGACAAGTGCTTTCCCAGCTCTTTTCCTGCACCACC |
| 295 | CKB | AGCCCGGCCCACACCCGACACCAGCCCTGCTGCTTCCTAACTTATTGCCT |
| | | GGGCAGTGCCCACCATGCACCCCTGATGTTCGCCGTCTGGCGAGCCCTT |
| | | AGCCTTGCTGTAGAGACTTCCGTCACCCTTGGTAGAGTTTATTTTTTTGAT |
| | | GGCTAAGATACTGCTGATGCTGAAATAAACTAGGGTTTTGGCCTGCCTGC |
| | | GTCTG |
| 296 | COPE | GGCTGGCCCAGAGCTGTCAGGACCATGAAGCCAGGACAGAGGCCAGGA |
| | | GCCAGCCCTGCAGCCCTCCCCACCCGGCATCCACCTGCATCCCCTCTGG |
| | | GGGCAGGAGCCCACCCCCA |
| 297 | DCTN3 | TAGCTGCTCCCCATCCCAAAGTGGGCCTGGGCAGTCAGGCTCCAGGGCC |
| | | CTATGCCAACCTGCCTTTGTTACAAGGCAGAGGAAGCTTTGTATTTATTGG |
| | | CTTCAAGGCCCACCTCTCTGTACTCTGGGCTCTAAAGTTGGAGGTCAGGT |
| | | TACCTGAGGTTTGCAATTTGCAACACCCACCCTCCCCCCAATCAGTGTTCT |
| | | TATTTCAGTGACAATAAACCATAGAGATGA |
| 298 | DCXR | GCTCCCTCCACACACCTCAAGCCCCATGCCGTGCTCATCCTACCCCCAAT |
| | | CCCTCCAATAAACCTGATTCTGCTGCCCAGACTATGTGCTATTCCTAGGCT |
| | | G |
| 299 | ECH1 | GAGCCCTCGCGTCCCAGGCCCCAGCCAGGGGGCCGGCCTTGTCCCGCC |
| | | TCATCCACAGAAAGGGAGGATGGGCGATGACAGTTGTTTCTATGCCTTCT |
| | | GACCCAGTTTCCCAGTTTATAACTTTATGACAATGAGTTTCTCAAGCCCAA |
| | | GGCCTTATCTTCACCCCACAAACAATAAAGCAAAGTAAAGAA |
| 300 | EIF3K | CTTCAGGTGTTTAATAAAGATGTGTTGACTCAGCC |
| 301 | FLNA | GTCTGGGGCCCGTGCCAGCCGGCAGCCCCCAAGCCTGCCCCGCTACCC |
| | | AAGCAGCCCCGCCCTCTTCCCCTCAACCCCGGCCCAGGCCGCCCTGGCC |
| | | GCCCGCCTGTCACTGCAGCCGCCCCTGCCCTGTGCCGTGCTGCGCTCAC |
| | | CTGCCTCCCCAGCCAGCCGCTGACCTCTCGGCTTTCACTTGGGCAGAGG |
| | | GAGCCA |
| 302 | FTL | GAGCCTTCTGAGCCCAGCGACTTCTGAAGGGCCCCTTGCAAAGTAATAGG |
| | | GCTTCTGCCTAAGCCTCTCCCTCCAGCCAATAGGCAGCTTTCTTAACTATC |
| | | CTAACAAGCCTTGGACCAAATGGAAATAAAGCTTTTTGATGC |
| 304 | GPX4 | ACAGACAAATTAGCCTGCTGGA |
| 305 | GSTK1 | GATTGCCCGGAGGAAGCAAACTCTTCGTATAAAAAAAGCAGGCCATCTGC |
| | | TTAACCCTTGGCTCCACCATAAGGCACTGGGACTCGGATTTCTCTATCTGA |
| | | TAGAGGTATTTTCTGTGGCCCTGGGAGCTGTCTGTCTTTCCCCTACCCCC |
| | | AAGGATGCCAGGAAGACGTCCACCATTAGCCATGTGGCAACCTTTACTTC |
| | | TATGCCTCACAAGTGCCTTTCAGAGAGCCCCAATTCTGCTTTCCCACAAAA |
| | | TAAACCTAATGCCATCAGGCAAAACA |
| 306 | KPTN | GCACCCATGCACCCACTCTTCCCAGACCTGGGGCGACTCATGGCGTGCT |
| | | CACCTCCAGCCGCTGGTGGGGGGTGTCCTGAAGGACAGGATGCTCTCCC |
| | | CAGACCTCCCCAGGGTGGTGAAGCTGTGCCCTGGGGCCCCAGCTAACCC |
| | | ACTCCTCTCTCTGTACGTCAATGTCCCTATTTGAAAGCAGGGATGATCCCA |
| | | TTCCACCCTGTCCACTCTGGATGAGAATTGGCCACCTGATGGTTATTTATA |
| | | CGTCCAGAAGAACATCATTTCCC |
| 307 | LGALS1 | AATCAGCCAGCCCATGGCCCCCAATAAAGGCAGCTGCCTCTGCTCCCTCT |
| | | GAACCAG |
| 308 | MAGED2 | GATTTTAGATATTGTTAATCCTGCCAGTCTTTCTCTTCAAGCCAGGGTGCA |
| | | TCCTCAGAAACCTACTCAACACAGCACTCTAGGCAGCCACTATCAATCAAT |
| | | TGAAGTTGACACTCTGCATTAAATCTATTTGCC |
| 309 | MIF | GAGCCGCAGGGACCCACGCTGTCTGCGCTGGCTCCACCCGGGAACCCG |
| | | CCGCACGCTGTGTTCTAGGCCCGCCCACCCCAACCTTCTGGTGGGGAGA |
| | | AATAAACGGTTTAGAGACTAGGA |
| 310 | MMP7 | AAACTTCAGGCAGAACATCCATTCATTCATTCATTGGATTGTATATCATTGT |
| | | TGCACAATCAGAATTGATAAGCACTGTTCCTCCACTCCATTTAGCAATTAT |
| | | GTCACCCTTTTTTATTGCAGTTGGTTTTTGAATGTCTTTCACTCCTTTTAAG |
| | | GATAAACTCCTTTATGGTGTGACTGTGTCTTATTCATCTATACTTGCAGTGG |
| | | GTAGATGTCAATAAATGTTACATACACAAATAAATAAAATGTTTATTCCATG |
| | | GTAAATTTA |
| 311 | MYL6B | GTGCTGCAGGTAGGGCCCTCCCACCCCTTCGCCGCGCCTTACGAGGCAA |
| | | GTCTCCCGCCCTTCTCTCAGCCAACATAGAAAAGCAGCGGAGTTCATTCT |
| | | GCTGTCCGGTTGCTGCACGGGCTCCAGCGCTTCGCAACTTTGGTTTTTTT |
| | | CCACAGATCCAGTGGGGTCCGGACACTGGGCCCCGCAGGCGAAAGCAC |
| | | GTTCCAGCCACCAGGAGGCCACCTATTGTTTCAAAATAAAGACTGGGTTC |
| | | CTCTCTTGG |
| 312 | NDUFA1 | GGAAGCATTTTCCTGATTGATGAAAAAAATAACTCAGTTATGGCCATCTAC |
| | | CCCTGCTAGAAGGTTACAGTGTATTATGTAGCATGCAATGTGTTATGTAGT |
| | | GCTTAATAAAAATAAAATGAAAAAAA |
| 313 | NDUFV1 | CCCACCACCCTGGCCTGCTGTCCTGCGTCTATCCATGTGGAATGCTGG |
| 314 | NPC2 | CAGGCCGTCCTCCACAAATCATATGTCCCAGATAAGCAGCCAGCAGGCAG |
| | | AAAGATCAGCTTAGCTCTGACTGCCAGTGGGTAAGGCCAAGAAGGAGCCT |
| | | GGCAGACAAATGGATGGCTGCTGGCCTTGAACATGGCAGAGCTGCTGGG |
| | | TGTGCCAGATCAAAAAGCAGAGAGCAGGCCAGCAGCAGCAGACTGTTCTC |
| | | AGCGCTGGAGAAATGCATTGCTCTGCCCACTACACCC |
| 315 | NUDT14 | GACTCCAGGGTTCTAGACAGAGGCCAGTCTTGGCCACTCACCCCACACCC |
| | | CGCGGACCCCAATAAAGGCTTGCATAGCTCCA |
| 316 | OST4 | AAGTGGCGCTTTCTCCGCCCCAGGTCTTTCCCCCTCTTTTCTAGGGTTCCA |
| | | GGACATAGTCTGAGGCAAGATGGAGGGTATGAGGGGCCTTCACACTTCAC |
| | | TTCATCCCTTCTACCCATCACAACATACAAAGCAACTACACCTGGATTTTTC |
| | | CAAACAACTTTTATTTCCTCAGAGTCTTCCTTAATCCTATGGAACAAGAAGC |
| | | TGCCACTGAATAGGGCCCAGTATAGGGGCTTGCTTTTCTACTCCCTCCCC |
| | | CCAATATAAAAATATAGACTTTTTTTTGTGGTCCCAAAA |
| 317 | PHPT1 | GCACTCCCAGCCCGGGGCCTGCTGCCTCCAGCAGCCACTTCAGAGCCCC |
| | | CGCCTTTGCCTGCACTCCTCTTGCAGGGCTGGCCCTGCCTGCTCCTGCG |
| | | GCAGCCTCTGGTGACGTGCTGTCCACCAGGCCTTGGAGACAGGCTAGCC |
| | | TGGCCACAGAATTAAACGTGTTGCCACA |
| 318 | PPDPF | CCTGAGCGGTTACCACCAGCCCCAGGCCTGCGGAGGCGCTAGTCCACCA |
| | | GAGCCCCTCC |
| 319 | PRDX5 | GGCCCTGGGCCAGATTACTTCCTCCACCCCTCCCTATCTCACCTGCCCAG |
| | | CCCTGTGCTGGGGCCCTGCAATTGGAATGTTGGCCAGATTTCTGCAATAA |
| | | ACACTTGTGGTTTGCGGCCA |
| 320 | RABAC1 | GGTGTCTTCTGGGACCTGCCGGCCTCCCGGGCCAGCTGCCCCACCCCTG |
| | | CCCATGCCTGTCCTGCACGGCTCTGCTGCTCGGGCCCACAGCGCCGTCC |
| | | CATCACAAGCCCGGGGAGGGATCCCGCCTTTGAAAATAAAGCTGTTATGG |
| | | GTGTCATTCAGGAAATA |
| 321 | RPL19 | AACCTCCCACTTTGTCTGTACATACTGGCCTCTGTGATTACATAGATCAGC |
| | | CATTAAAATAAAACAAGCCTTAATCTGC |
| 322 | RPL36 | GCCCCTCCCCTGCCCTCTCCCTGAAATAAAGAACAGCTTGACAGAAGCCC |
| | | TGGCTCTCCTGCTGTCCGTGGGTGGGTGTGGGTGTGTCGGGGGCCCGCA |
| | | GTCCCCTGTCTGGTGCCCGCTCTGAGCCACACCCTCTCCGGGTGCTGCC |
| | | TGGTCGTGAATCAAAAGCCGTGGCCCGCCCACCCTTCCCGGGGCAGCAG |
| | | GTGAGGAAGCCGCCGTACTGCAAATGACTTTAATCATTAAATAGCTTCTAT |
| | | GCCACAC |
| 323 | RPLPO | TCACCAAAAAGCAACCAACTTAGCCAGTTTTATTTGCAAAACAAGGAAATA |
| | | AAGGCTTACTTCTTTAAAAAGTCTCTGGACTCTTAA |
| 324 | RPS11 | GGCTGGACATCGGCCCGCTCCCCACAATGAAATAAAGTTATTTTCTCATTC |
| | | CCA |
| 325 | RPS21 | ATCTGCTGGCAGAGTAGTTTGAGCCAGCTGGACTGGGCTGGCCGCCTGC |
| | | CGCTTCTTGAGGGTGGAAGAGGGGTGCTCTGAGAAGACACTCAGGCAGC |
| | | AGACTCTGCCTCTCAC |
| 326 | SSR4 | GGGCGGCACCCCAGCCCTGCCCTTGCTTCCTTCAATAAACATCACAGGAC |
| | | CTGGGACTGCACAGGACCTGGG |
| 327 | TMEM258 | GCACCCAAGGGTAACAACCAGATGGCTTCACTGAAACCTGCTTTTGTAAAT |
| | | TACTTTTTTTTACTGTTGCTGGAAGTGTCCCACCTGCTGCTCATAATAAATG |
| | | CAGATGTATAGCAGTCCTGTCTGGGGCCCATTCTGCAGGGGTGGTGGGT |
| | | GCTGGGACCTACATTGCTTCCCCAATCTCTTCCCTTTCCCATTTCTCATCT |
| | | GCCTTTCACAGTTCCTGCTGCTGATCTACTTAAGGGGGATGAGATTTGTGT |
| | | AGCACCGGTGTTTTGTCATTGGAATTTAA |
| 328 | TRAPPC6A | GCCTGCCTCGCACCTGCTGAGCCGCACTGCTGCCCCTGGCCTCACCGGC |
| | | CTCAGGAGACGGTGGGGCCGCTGCATTTCGGGGTGGTCTTGGGAATCCC |
| | | AGGCCCTAGGGGTCACATTTGCTCAGGAAGTGGGTATCAAATTGAGGTGG |
| | | GGGTGTCAGAGGAGGCAAAGGGGTCCCAGCTGCGGTCAGGACTGTGGT |
| | | GGGATTCCCGCCTCGGCAGCGTCACTGCCCAGTAGCTGCTCAATAAAGG |
| | | CTGTGTGTGTGGAATCTGA |
| 329 | UCHL1 | TTTCCCCTCTTCCCTTCAACATGAAAA |
| 330 | YIF1B | GCGCGCCCGCTGAACCTCCCGCTGCTGCTGCTGCTGCTGGGGGCCACTG |
| | | TGGCCGCCGAACTCATCTCCTGCCTGCAGGCCCCAAGGTCCACCCTGTCT |
| | | GGCCACAGGCACCGCCTCCATCCCATGTCCCGCCCAGCCCCGCCCCCAA |
| | | CCCAAGGTGCTGAGAGATCTCCAGCTGCACAGGCCACCGCCCCAGGGCG |
| | | TGGCCGCTGTTACAGAAACAATAAACCCTGATGGGCA |
| 331 | COX411 | GAGATGCTGGCCTGCGCCTGCACCTGCGCCTGGCTCTGTCACCGCCATG |
| | | CAACTCCATGCCTATTTACTGGAAACCTGTTATGCCAAACAGTTGTACCAC |
| | | TGCTAATAAATGACCAGTTTACCTGAAACCCTTTGTGATCAGTTCTTTAATG |
| | | ATACCTAAATGAAAGCTA |
| 332 | SERINC2 | GGCAGCCTCACAGCCTGCCATCTGGTGCCTCCTGCCACCTGGTGCCTCT |
| | | CGGCTCAGTGACAGCCAACCTGCCCCCTCCCCACACCAATCAGCCAGGC |
| | | TGAGCCCCCACCCCTGCCCCAGCTCCAGGACCTGCCCCTGAGCCGGGCC |
| | | TTCTAGTCGTAGTGCCTTCAGGGTCCGAGGAGCATCAGGCTCCTGCAGAG |
| | | CCCCATCCCCCCGCCACACCCACACGGTGGAGCTGCCTCTTCCTTCCCCT |
| | | CCTCCCTGTTGCCCATACTCAGCATCTCGGATGAAAGGGCTCCCTTGTCC |
| | | TCAGGCTCCACGGGAGCGGGGCTGCTGGAGAGAGCGGGGAACTCCCAC |
| | | CACAGTGGGGCATCCGGCACTGAAGCCCTGGTGTTCCTGGTCACGTCCC |
| | | CCAGGGGACCCTGCCCACTTCCTGGACTTCGTGCCTTACTGAGTCTCTAA |
| | | GACTTTTTCTAATAAACAAGCCAGTGCGTGTACC |
| 333 | TSPO | GTGCCCGGCCCACCAGGGACTGCAGCTGCACCAGCAGGTGCCATCACGC |
| | | TTGTGATGTGGTGGCCGTCACGCTTTCATGACCACTGGGCCTGCTAGTCT |
| | | GTCAGGGCCTTGGCCCAGGGGTCAGCAGAGCTTCAGAGGTGGCCCCACC |
| | | TGAGCCCCCACCCGGGAGCAGTGTCCTGTGCTTTCTGCATGOTTAGAGCA |
| | | TGTTCTTGGAACATGGAATTTTATAAGCTGAATAAAGTTTTTGACTTCCTTT |
| | | A |

### Untranslated Regions

In some embodiments, a circular or linear polyribonucleotide includes untranslated regions (UTRs). In some embodiments, the stability element may include a UTR. In some embodiments, the translation enhancer may include a UTR. In some embodiments, the UTR is a 3′ UTR. In some embodiments, the UTR is a 5′ UTR. UTRs of a genomic region including a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for the first expression sequence is the same as or continuous with or overlapping with another UTR for a second expression sequence. In some embodiments, the intron is a human intron. In some embodiments, the intron is a full-length human intron, e.g., ZKSCAN1.

Exemplary untranslated regions are described in paragraphs [0197]-[201] of International Patent Publication No. WO2019/118919, which is hereby incorporated by reference in its entirety.

In some embodiments, a circular polyribonucleotide includes a poly-A sequence. Exemplary poly-A sequences are described in paragraphs [0202]-[0205] of International Patent Publication No. WO2019/118919, which is hereby incorporated by reference in its entirety. In some embodiments, a circular polyribonucleotide lacks a poly-A sequence.

In some embodiments, a circular or linear polyribonucleotide includes a UTR with one or more stretches of Adenosines and Uridines embedded within. These AU rich signatures may increase turnover rates of the expression product.

Introduction, removal, or modification of UTR AU rich elements (AREs) may be useful to modulate the stability, or immunogenicity (e.g., the level of one or more markers of an immune or inflammatory response) of the circular or linear polyribonucleotide. When engineering specific circular polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

It should be understood that any UTR from any gene may be incorporated into the respective flanking regions of the circular polyribonucleotide.

In some embodiments, a circular polyribonucleotide lacks a 5′-UTR and is competent for protein expression from its one or more expression sequences. In some embodiments, the circular or linear polyribonucleotide lacks a 3′-UTR and is competent for protein expression from its one or more expression sequences. In some embodiments, the circular or linear polyribonucleotide lacks a poly-A sequence and is competent for protein expression from its one or more expression sequences. In some embodiments, the circular or linear polyribonucleotide lacks a termination element and is competent for protein expression from its one or more expression sequences. In some embodiments, the circular or linear polyribonucleotide lacks an internal ribosomal entry site and is competent for protein expression from its one or more expression sequences. In some embodiments, the circular or linear polyribonucleotide lacks a cap and is competent for protein expression from its one or more expression sequences. In some embodiments, the circular or linear polyribonucleotide lacks a 5′-UTR, a 3′-UTR, and an IRES, and is competent for protein expression from its one or more expression sequences. In some embodiments, the circular or linear polyribonucleotide includes one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

In some embodiments, a circular or linear polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular or linear polyribonucleotide lacks a termination element. In some embodiments, the circular or linear polyribonucleotide lacks an internal ribosomal entry site. In some embodiments, the circular or linear polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein. In some embodiments, the circular polyribonucleotide lacks a 5′ cap.

### Spacer Elements

In some embodiments, the polyribonucleotide includes one or more spacer elements. For example, either the first expression augmenting element or the second expression augmenting element may include a spacer element fused to a translation enhancer or a spacer element fused to a stability element. In some embodiments, the spacer may be fused to the 5′ end of the translation enhancer or stability element. In some embodiments, the spacer may be fused to the 3′ end of the translation enhancer or stability element. In some embodiments, a spacer element is a translation enhancer. Not all spacers are translation enhancers. In some embodiments, a spacer element is a stability element. Not all spacers are stability elements. A spacer element refers to any contiguous nucleotide sequence (e.g., of one or more nucleotides) that provides distance or flexibility between two adjacent polynucleotide regions. Spacer elements may be present in between any of the nucleic acid elements described herein. Spacer element may also be present within a nucleic acid element described herein. The circular polyribonucleotide may include a first post-circularization element; a first spacer element having a length of at least 100 ribonucleotides; a polyribonucleotide cargo; a second spacer element; and a second post-circularization element; and wherein the first post-circularization element and the second post-circularization element together form a circularization junction. The circular polyribonucleotide may include a first post-circularization element; a first spacer element; a polyribonucleotide cargo; a second spacer element having a length of at least 100 ribonucleotides; and a second post-circularization element; and wherein the first post-circularization element and the second post-circularization element together form a circularization junction.

In some embodiments, the spacer element has a length of at least 100 ribonucleotides. The spacer element may have a length of between 100 to 500 (e.g., between 100 and 400, 100 and 300, 100 and 200, 200 and 500, 300 and 500, 400 and 500, 200 and 400, or 200 and 300) ribonucleotides. For example, the spacer element not including an expression augmenting element may have a length of between 120 and 500 (e.g., between 120 and 400, 120 and 300, 120 and 200, 120 and 150, 150 and 500, 200 and 500, 250 and 500, 300 and 500, 400 and 500, or 350 and 500) ribonucleotides. In some embodiments, the spacer element includes between 110 and 500 (e.g., between 110 and 400, 110 and 300, 110 and 200, 110 and 150, 150 and 500, 200 and 500, 250 and 500, 300 and 500, 400 and 500, or 350 and 500) ribonucleotides. In some embodiments, the spacer element includes between 100 and 300 (e.g., between 100 and 280, 100 and 260, 100 and 240, 100 and 220, 100 and 200, 100 and 180, 100 and 160, 100 and 140, 100 and 120, 110 and 300, 120 and 300, 140 and 300, 160 and 300, 180 and 300, 200 and 300, 220 and 300, 240 and 300, 260 and 300, or 280 and 300) ribonucleotides. In some embodiments, the spacer element has a length of between 200 and 500 (e.g., between 200 and 450, 200 and 400, 200 and 350, 200 and 300, 200 and 250, 250 and 500, 300 and 500, 350 and 500, 400 and 500, 450 and 500, 300 and 500, or 300 and 400) ribonucleotides. In some embodiments, the spacer element has at least 50 ribonucleotides. For example, the spacer element may have between 50 and 500 ribonucleotides (e.g., 50 and 100, 50 and 150, 50 and 200, 50 and 250, 50 and 300, 50 and 350, 50 and 400, 50 and 450, 100 and 500, 150 and 500, 200 and 500, 250 and 500, 300 and 500, 350 and 500, 400 and 500, and 450 and 500).

In some embodiments, the spacer element has a length of about 50 (e.g., 45, 46, 47, 48, 49, 50, 51, 52, 53, 54, or 55) ribonucleotides. In some embodiments, the spacer element has a length of about 80 (e.g., 72, 73, 74, 75, 76, 77, 78, 79, 80, 81, 82, 83, 84, 85, 86, 87, or 88) ribonucleotides. In some embodiments, the spacer element has a length of about 100 (e.g., 90, 91, 92, 93, 94, 95, 96, 97, 98, 99, 100, 101, 102, 103, 104, 105, 106, 107, 108, 109, or 110) ribonucleotides. In some embodiments, the spacer element has a length of about 120 (e.g., 108, 109, 110, 111, 112, 113, 114, 115, 116, 117, 118, 119, 120, 121, 122, 123, 124, 125, 126, 127, 128, 129, 130, 131, or 132) ribonucleotides. In some embodiments, the spacer element has a length of about 150 (e.g., 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, or 165) ribonucleotides. In some embodiments, the spacer element has a length of about 200 (e.g., 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, or 220) ribonucleotides.

In some embodiments, the polyribonucleotide includes a first spacer element and a second spacer element, wherein the first and second spacer are the same length. In some embodiments, the first spacer element and the second spacer element are about the same length.

In some embodiments, the polyribonucleotide includes a first spacer element and a second spacer element, wherein the first spacer element and the second spacer elements are different lengths. In some embodiments, the difference between the length of the first spacer element and the length of the second region is 0 to 100 (e.g., 0 to 95, 0 to 90, 0 to 85, 0 to 80, 0 to 75, 0 to 70, 0 to 65, 0 to 60, 0 to 55, 0 to 50, 0 to 45, 0 to 40, 0 to 35, 0 to 30, 0 to 25, 0 to 20, 0 to 15, 0 to 10, 0 to 5, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, or 95 to 100) nucleotides. In some embodiments, the difference between the length of the first spacer element and the length of the second spacer element is 0 to 50 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotides. In some embodiments, the length of the first spacer is about 50 (e.g., 45, 46, 47, 48, 49, 50, 51, 52, 53, 54, or 55) ribonucleotides, and the length of the second spacer is about 120 (e.g., 108, 109, 110, 111, 112, 113, 114, 115, 116, 117, 118, 119, 120, 121, 122, 123, 124, 125, 126, 127, 128, 129, 130, 131, or 132) ribonucleotides. In some embodiments, the length of the first spacer is about 120 (e.g., 108, 109, 110, 111, 112, 113, 114, 115, 116, 117, 118, 119, 120, 121, 122, 123, 124, 125, 126, 127, 128, 129, 130, 131, or 132) ribonucleotides, and the length of the second spacer is about 50 (e.g., 45, 46, 47, 48, 49, 50, 51, 52, 53, 54, or 55) ribonucleotides.

In some embodiments, a spacer element may be a polyA based spacer, or a polyA based spacer element. In some embodiments, a first spacer is a polyA based spacer, or a polyA based spacer element. In some embodiments a second spacer is a polyA based spacer, or a polyA based spacer element. In some embodiments, the first spacer and the second spacer may be a polyA based spacer, or a polyA based spacer element. A polyA based spacer, or a polyA based spacer element is equivalent to a polyA region (e.g., a polyAC region, polyAU region, polyAG region, or polyAT region) as described herein.

The first spacer element may consist of a polyA region comprising 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine residues. The first spacer element may consist of a polyAC region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine or cytosine residues. The adenosine and cytosine residues may be present in any ratio to one another. In some embodiments, the first spacer element may consist of a polyAU region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine or uridine residues. In some embodiments, the first spacer element may consist of a polyAG region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine or guanosine residues. The adenosine and guanosine residues may be present in any ratio to one another. In some embodiments, the first spacer element may consist of a polyAT region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine or thymidine residues. The adenosine and thymidine residues may be present in any ration to one another. In some embodiments, the first spacer element includes a polyAT region, wherein the polyAT region includes between 100 ribonucleotides and 150 ribonucleotides (e.g., 105, 110, 115, 120, 125, 130, 135, 140, 145, or 150 ribonucleotides).

The second spacer element may consist of a polyA region comprising 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine residues. The second spacer element may consist of a polyAC region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine or cytosine residues. In some embodiments, the second spacer element may consist of a polyAU region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine or uridine residues. The adenosine and uridine residues may be present in any ratio to one another. In some embodiments, the second spacer element may consist of a polyAG region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine or guanosine residues. The adenosine and guanosine residues may be present in any ratio to one another. In some embodiments, the first spacer element may consist of a polyAT region comprising between 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine or thymidine residues. In some embodiments, the second spacer element includes a polyAT region, wherein the polyAT region includes between 100 ribonucleotides and 150 ribonucleotides (e.g., 105, 110, 115, 120, 125, 130, 135, 140, 145, or 150 ribonucleotides). The adenosine and thymidine residues may be present in any ratio to one another.

In some embodiments, the spacer element sequences can be a polyA region (e.g., polyAT, polyAU, polyAC, or polyAG) or a random sequence. In some embodiments, the spacer element may be referred to based on a combination of nucleotide content and length. For example, an AU120 spacer element may consist of a polyAU region comprising between 80% to 100% adenosine or uridine residues and 120 nucleotides in length.

In some embodiments, the spacer element has a nucleic acid sequence having at least 85% (e.g., at least 85%, 86%, 87%, 88%, 89%, 90%, 91%, 92%, 93%, 94%, 95%, 96%, 97%, 98%, 99%, or 100%) sequence identity to any one of the sequences described in TABLE 10. In some embodiments, the spacer element has a nucleic acid sequence having at least 90% (e.g., at least 90%, 91%, 92%, 93%, 94%, 95%, 96%, 97%, 98%, 99%, or 100%) sequence identity to any one of the sequences described in TABLE 10. In some embodiments, the spacer element has a nucleic acid sequence having at least 95% (e.g., at least 95%, 96%, 97%, 98%, 99%, or 100%) sequence identity to any one of the sequences described in TABLE 10. In some embodiments, the spacer element has a nucleic acid sequence of any one of the sequences described in TABLE 10.

TABLE 10
Spacer Element Sequences

| SEQ ID | | |
|---|---|---|
| NO: | Name | Sequence |
| 335 | AU120 | AAAAAAAUAAAAAAAAAUUAAAAAAAAAAUUUAAAAAAAAAUUAAAAAAAUUUAAAAA |
| | | AAAUUAAAAAAAAAAUAAAAAAUUAAAAAAAUAAAAAAAAAAUUUAAAAAAAAAUAAA |
| | | AAAA |
| 336 | AT120 | AAAAAAATAAAAAAAAATTAAAAAAAAAATTTAAAAAAAAATTAAAAAAATTTAAAAAAA |
| | | ATTAAAAAAAAAATAAAAAATTAAAAAAATAAAAAAAAAATTTAAAAAAAAATAAAAAAA |
| 337 | AC120 | AAAAAAACAAAAAAAAACCAAAAAAAAAACCCAAAAAAAAACCAAAAAAACCCAAAAA |
| | | AAACCAAAAAAAAAACAAAAAACCAAAAAAAAAAAAAAAAACCCAAAAAAAAACAAA |
| | | AAAA |
| 338 | AC50 | AAAAAAACAAAAAAAAACCAAAAAAAAAACCCAAAAAAAAACCAAAAAAA |

Exemplary spacer element sequences are described in paragraphs [0293]-[0302] of International Patent Publication No. WO2019/118919, which is hereby incorporated by reference in its entirety.

### Internal Ribosome Entry Sites

In some embodiments, a circular or linear polyribonucleotide described herein includes one or more internal ribosome entry site (IRES) elements. In some embodiments, the IRES is operably linked to one or more expression sequences (e.g., each IRES is operably linked to one or more expression sequences, where each expression sequence optionally encodes a polypeptide. In embodiments, the IRES is located between a heterologous promoter and the 5′ end of a coding sequence.

A suitable IRES element to include in a polyribonucleotide includes an RNA sequence capable of engaging a eukaryotic ribosome. In some embodiments, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt.

In some embodiments, the IRES element is from the DNA of an organism including, but not limited to, a virus, a mammal, and a Drosophila. Such viral DNA may be from, but is not limited to, picomavirus complementary DNA (cDNA), with encephalomyocarditis virus (EMCV) cDNA and poliovirus cDNA. In one embodiment, an IRES element is from an Antennapedia gene from Drosophila melanogaster.

In some embodiments, the IRES sequence is an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, human poliovirus 1, Plautia stall intestine virus, Kashmir bee virus, Human rhinovirus 2 (HRV-2), Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picorna-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus (AEV), Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-I, Human BCL2, Human BiP, Human c-IAPI, Human c-myc, Human elF4G, Mouse NDST4L, Human LEF1, Mouse HIF1 alpha, Human n.myc, Mouse Gtx, Human p27kipl, Human PDGF2/c-sis, Human p53, Human Pim-I, Mouse Rbm3, Drosophila reaper, Canine Scamper, Drosophila Ubx, Human UNR, Mouse UtrA, Human VEGF-A, Human XIAP, Salivirus, Cosavirus, Parechovirus, Drosophila hairless, S. cerevisiae TFIID, S. cerevisiae YAP1, Human c-src, Human FGF-I, Simian picomavirus, Turnip crinkle virus, Aichivirus, Crohivirus, Echovirus 11, an aptamer to elF4G, Coxsackievirus B3 (CVB3) or Coxsackievirus A (CVB1/2). In yet another embodiment, the IRES is an IRES sequence of Coxsackievirus B3 (CVB3). In a further embodiment, the IRES is an IRES sequence of Encephalomyocarditis virus. In a further embodiment, the IRES is an IRES sequence of Theiler's encephalomyelitis virus.

In some embodiments, the IRES sequence has a modified sequence in comparison to the wild-type IRES sequence. In some embodiments, when the last nucleotide of the wild-type IRES is not a cytosine nucleic acid residue, the last nucleotide of the wild-type IRES sequence is modified such that it is a cytosine residue. For example, the IRES sequence may be a CVB3 IRES sequence wherein the terminal adenosine residue is modified to cytosine residue. In some embodiments, the modified CVB3 IRES may have the nucleic acid sequence of:

(SEQ ID NO: 184)
UUAAAACAGCCUGUGGGUUGAUCCCACCCACAGGCCCAUUGGGCGCUAGCACUCUGG
UAUCACGGUACCUUUGUGCGCCUGUUUUAUACCCCCUCCCCCAACUGUAACUUAGAA
GUAACACACACCGAUCAACAGUCAGCGUGGCACACCAGCCACGUUUUGAUCAAGCACU
UCUGUUACCCCGGACUGAGUAUCAAUAGACUGCUCACGCGGUUGAAGGAGAAAGCGU
UCGUUAUCCGGCCAACUACUUCGAAAAACCUAGUAACACCGUGGAAGUUGCAGAGUG
UUUCGCUCAGCACUACCCCAGUGUAGAUCAGGUCGAUGAGUCACCGCAUUCCCCACG
GGCGACCGUGGCGGUGGCUGCGUUGGCGGCCUGCCCAUGGGGAAACCCAUGGGACG
CUCUAAUACAGACAUGGUGCGAAGAGUCUAUUGAGCUAGUUGGUAGUCCUCCGGCCC
CUGAAUGCGGCUAAUCCUAACUGCGGAGCACACACCCUCAAGCCAGAGGGCAGUGUG
UCGUAACGGGCAACUCUGCAGCGGAACCGACUACUUUGGGUGUCCGUGUUUCAUUUU
AUUCCUAUACUGGCUGCUUAUGGUGACAAUUGAGAGAUCGUUACCAUAUAGCUAUUG
GAUUGGCCAUCCGGUGACUAAUAGAGCUAUUAUAUAUCCCUUUGUUGGGUUUAUACC
ACUUAGCUUGAAAGAGGUUAAAACAUUACAAUUCAUUGUUAAGUUGAAUACAGCAAC

In some embodiments, the IRES sequence is an Enterovirus 71 (EV17) IRES. In some embodiments, the terminal guanosine residue of the EV17 IRES sequence is modified to a cytosine residue. In some embodiments, the modified EV71 IRES may have the nucleic acid sequence of:

(SEQ ID NO: 185)
UUAAAACAGCUGUGGGUUGUCACCCACCCACAGGGUCCACUGGGCGCUAGUACACUG
GUAUCUCGGUACCUUUGUACGCCUGUUUUAUACCCCCUCCCUGAUUUGCAACUUAGA
AGCAACGCAAACCAGAUCAAUAGUAGGUGUGACAUACCAGUCGCAUCUUGAUCAAGCA
CUUCUGUAUCCCCGGACCGAGUAUCAAUAGACUGUGCACACGGUUGAAGGAGAAAAC
GUCCGUUACCCGGCUAACUACUUCGAGAAGCCUAGUAACGCCAUUGAAGUUGCAGAG
UGUUUCGCUCAGCACUCCCCCCGUGUAGAUCAGGUCGAUGAGUCACCGCAUUCCCCA
CGGGCGACCGUGGCGGUGGCUGCGUUGGCGGCCUGCCUAUGGGGUAACCCAUAGGA
CGCUCUAAUACGGACAUGGCGUGAAGAGUCUAUUGAGCUAGUUAGUAGUCCUCCGGC
CCCUGAAUGCGGCUAAUCCUAACUGCGGAGCACAUACCCUUAAUCCAAAGGGCAGUG
UGUCGUAACGGGCAACUCUGCAGCGGAACCGACUACUUUGGGUGUCCGUGUUUCUU
UUUAUUCUUGUAUUGGCUGCUUAUGGUGACAAUUAAAGAAUUGUUACCAUAUAGCUA
UUGGAUUGGCCAUCCAGUGUCAAACAGAGCUAUUGUAUAUCUCUUUGUUGGAUUCAC
ACCUCUCACUCUUGAAACGUUACACACCCUCAAUUACAUUAUACUGCUGAACACGAAG
CGGCCACC

In some embodiments, the IRES sequence is a synthetic IRES. A “synthetic IRES” is an IRES that is modified relative to a wildtype IRES in order to modulate its structure and/or activity. For example, in some embodiments, an IRES that is modified to incorporate an aptamer sequence is a synthetic IRES. In some embodiments, the polyribonucleotide includes at least one IRES flanking at least one (e.g., 2, 3, 4, 5 or more) expression sequence. In some embodiments, the IRES flanks both sides of at least one (e.g., 2, 3, 4, 5 or more) expression sequence. In some embodiments, the polyribonucleotide includes one or more IRES sequences on one or both sides of each expression sequence, leading to separation of the resulting peptide(s) and or polypeptide(s). For example, a polyribonucleotide described herein may include a first IRES operably linked to a first expression sequence and a second IRES operably linked to a second expression sequence.

In some embodiments, a polyribonucleotide described herein includes an IRES (e.g., an IRES operably linked to a coding region). For example, the polyribonucleotide may include any IRES as described in Chen et al. Nature Biotechnology 41:262-272, 2023, Chen et al. Mol. Cell 81 (20): 4300-4318, 2021; Jopling et al. Oncogene 20:2664-2670, 2001; Baranick et al. PNAS 105 (12): 4733-4738, 2008; Lang et al. Molecular Biology of the Cell 13 (5): 1792-1801, 2002; Dorokhov et al. PNAS 99(8):5301-5306, 2002; Wang et al. Nucleic Acids Research 33(7):2248-2258, 2005; Petz et al. Nucleic Acids Research 35(8):2473-2482, 2007, Chen et al. SCIENCE 268:415-417, 1995; Fan et al. NATURE COMMUNICATION 13(1):3751-3765, 2022, International Publication No. WO2021/263124, and International Publication No. WO2022/271965 each of which is hereby incorporated by reference in their entirety.

### Signal Sequences

In some embodiments, polypeptides expressed from a circular or linear polyribonucleotide disclosed herein include a secreted protein, for example, a protein that naturally includes a signal sequence, or one that does not usually encode a signal sequence but is modified to contain one. In some embodiments, the polypeptide(s) includes a secretion signal. For example, the secretion signal may be the naturally encoded secretion signal for a secreted protein. In another example, the secretion signal may be a modified secretion signal for a secreted protein. In other embodiments, the polypeptide(s) do not include a secretion signal.

In some embodiments, a polyribonucleotide encodes multiple copies of the same polypeptide (e.g., one, two, three, four, five, six, seven, eight, nine, ten, or more). In some embodiments, at least one copy of the polypeptide includes a signal sequence and at least one copy of the polypeptide does not include a signal sequence. In some embodiments, a circular polyribonucleotide encodes plurality of polypeptides, where at least one of the plurality of polypeptides includes a signal sequence and at least one copy of the plurality of polypeptides does not include a signal sequence.

In some embodiments, the signal sequence is a wild-type signal sequence that is present on the N-terminus of the corresponding wild-type polypeptide, e.g., when expressed endogenously. In some embodiments, the signal sequence is heterologous to the polypeptide, e.g., is not present when the wild-type polypeptide is expressed endogenously. A polyribonucleotide sequence encoding an polypeptide may be modified to remove the nucleotide sequence encoding a wild-type signal sequence and/or add a sequence encoding a heterologous signal sequence.

A polypeptide encoded by a may include a signal sequence that directs the polypeptide to the secretory pathway. In some embodiments, the signal sequence may direct the polypeptide to reside in certain organelles (e.g., the endoplasmic reticulum, Golgi apparatus, or endosomes). In some embodiments, the signal sequence directs the polypeptide to be secreted from the cell. For secreted proteins, the signal sequence may be cleaved after secretion, resulting in a mature protein. In other embodiments, the signal sequence may become embedded in the membrane of the cell or certain organelles, creating a transmembrane segment that anchors the protein to the membrane of the cell, endoplasmic reticulum, or Golgi apparatus. In certain embodiments, the signal sequence of a transmembrane protein is a short sequence at the N-terminal of the polypeptide. In other embodiments, the first transmembrane domain acts as the first signal sequence, which targets the protein to the membrane.

In some embodiments, the secretion signal is human interleukin-2 (IL-2) secretion signal. In some embodiments, the IL-2 secretion signal has an amino acid sequence of at least 90% sequence identity to MYRMQLLSCIALSLALVTNS (SEQ ID NO: 186). In some embodiments, the IL2 secretion signal has an amino acid sequence of at least 95% sequence identity to SEQ ID NO: 186. In some embodiments, the IL-2 secretion signal has an amino acid sequence of at least 99% sequence identity to SEQ ID NO: 186. In some embodiments, the IL-2 secretion signal has an amino acid sequence of 100% sequence identity to SEQ ID NO: 186.

In some embodiments, the secretion signal is Gaussia luciferase secretion signal. In some embodiments, the Gaussia luciferase secretion signal has an amino acid sequence of at least 90% sequence identity of MGVKVLFALICIAVAEAK (SEQ ID NO: 187). In some embodiments, the Gaussia luciferase secretion signal has an amino acid sequence of at least 95% sequence identity of SEQ ID NO: 187. In some embodiments, the Gaussia luciferase secretion signal has an amino acid sequence of at least 99% sequence identity of SEQ ID NO: 187. In some embodiments, the Gaussia luciferase secretion signal has an amino acid sequence of 100% sequence identity of SEQ ID NO: 187.

In some embodiments, the secretion signal is an EPO (e.g., a human EPO) secretion signal. In some embodiments, the EPO secretion signal has an amino acid sequence of at least 90% sequence identity of MGVHECPAWLWLLLSLLSLPLGLPVLGA (SEQ ID NO: 188). In some embodiments, the EPO secretion signal has an amino acid sequence of at least 95% sequence identity of SEQ ID NO: 188. In some embodiments, the 88. In some embodiments, the EPO secretion signal has an amino acid sequence of 100% sequence identity of SEQ ID NO: 188.

In some embodiments, the secretion signal is a wildtype SARS-COV-2 secretion signal. In some embodiments, the wildtype SARS-COV-2 secretion signal has an amino acid sequence of at least 90% sequence identity of MFVFLVLLPLVSS (SEQ ID NO: 189). In some embodiments, the wildtype SARS-COV-2 secretion signal has an amino acid sequence of at least 95% sequence identity of SEQ ID NO: 189. In some embodiments, the wildtype SARS-COV-2 secretion signal has an amino acid sequence of at least 99% sequence identity of SEQ ID NO: 189. In some embodiments, the wildtype SARS-COV-2 secretion signal has an amino acid sequence of 100% sequence identity of SEQ ID NO: 189.

In some embodiments, a polypeptide encoded by a polyribonucleotide includes either a secretion signal sequence, a transmembrane insertion signal sequence, or does not include a signal sequence.

### Regulatory Elements

In some embodiments, the polyribonucleotide described herein (e.g., the polyribonucleotide cargo of the polyribonucleotide) includes one or more regulatory elements. In some embodiments, the polyribonucleotide includes a regulatory element, e.g., a sequence that modifies expression of an expression sequence within the polyribonucleotide.

A regulatory element may include a sequence that is located adjacent to an expression sequence that encodes an expression product. A regulatory element may be operably linked to the adjacent sequence. A regulatory element may increase an amount of product expressed as compared to an amount of the expressed product when no regulatory element is present. A regulatory element may be used to increase the expression of one or more polypeptide(s) encoded by a polyribonucleotide. Likewise, a regulatory element may be used to decrease the expression of one or more polypeptide(s) encoded by a polyribonucleotide. In some embodiments, a regulatory element is used to increase expression of polypeptide and another regulatory element is used to decrease expression of another polypeptide on the same polyribonucleotide. In addition, one regulatory element can increase an amount of product expressed for multiple expression sequences attached in tandem. Hence, one regulatory element can enhance the expression of one or more expression sequences. Multiple regulatory elements can also be used, for example, to differentially regulate expression of different expression sequences.

In some embodiments, the regulatory element is a translation modulator. A translation modulator can modulate translation of the expression sequence in the polyribonucleotide. A translation modulator can be a translation enhancer or suppressor. In some embodiments, the polyribonucleotide includes at least one translation modulator adjacent to at least one expression sequence. In some embodiments, the polyribonucleotide includes a translation modulator adjacent to each expression sequence. In some embodiments, the translation modulator is present on one or both sides of each expression sequence, leading to separation of the expression products, e.g., peptide(s) and or polypeptide(s).

In some embodiments, the regulatory element is a microRNA (miRNA) or a miRNA binding site.

In some embodiments, a regulatory element as provided herein includes a selective translation sequence. As used herein, the term “selective translation sequence” refers to a nucleic acid sequence that selectively initiates or activates translation of an expression sequence in the polyribonucleotide, for instance, certain riboswitch aptazymes. A regulatory element can also include a selective degradation sequence. As used herein, the term “selective degradation sequence” refers to a nucleic acid sequence that initiates degradation of the polyribonucleotide, or an expression product of the polyribonucleotide. In some embodiments, the regulatory element is a translation modulator. A translation modulator can modulate translation of the expression sequence in the polyribonucleotide. A translation modulator can be a translation enhancer or suppressor. In some embodiments, a translation initiation sequence can function as a regulatory element.

In some embodiments, a polyribonucleotide produces stoichiometric ratios of expression products. Rolling circle translation continuously produces expression products at substantially equivalent ratios. In some embodiments, the polyribonucleotide has a stoichiometric translation efficiency, such that expression products are produced at substantially equivalent ratios. In some embodiments, the polyribonucleotide has a stoichiometric translation efficiency of multiple expression products, e.g., products from 2, 3, 4, 5, 6, 7, 8, 9, 10, 11, 12, or more expression sequences. In some embodiments, the polyribonucleotide produces substantially different ratios of expression products. For example, the translation efficiency of multiple expression products may have a ratio of 1:10,000; 1:7000, 1:5000, 1:1000, 1:700, 1:500, 1:100, 1:50, 1:10, 1:5, 1:4, 1:3 or 1:2. In some embodiments, the ratio of multiple expression products may be modified using a regulatory element.

Further examples of regulatory elements are described in paragraphs [0154]-[0161] of International Patent Publication No. WO2019/118919, which is hereby incorporated by reference in its entirety.

### Cleavage Domains

A circular or linear polyribonucleotide of the disclosure can include a cleavage domain (e.g., a stagger element or a cleavage sequence).

As used herein, the term “stagger element” is a moiety, such as a nucleotide sequence, that induces ribosomal pausing during translation. In some embodiments, the stagger element is a non-conserved sequence of amino-acids with a strong alpha-helical propensity followed by the consensus sequence -D(V/I)ExNPGP (SEQ ID NO: 190), where x=any amino acid. In some embodiments, the stagger element may include a chemical moiety, such as glycerol, a non-nucleic acid linking moiety, a chemical modification, a modified nucleic acid, or any combination thereof.

In some embodiments, a circular or linear polyribonucleotide includes at least one stagger element adjacent to an expression sequence. In some embodiments, the circular or linear polyribonucleotide includes a stagger element adjacent to each expression sequence. In some embodiments, the stagger element is present on one or both sides of each expression sequence, leading to separation of the expression products. In some embodiments, the stagger element is a portion of the one or more expression sequences. In some embodiments, the circular or linear polyribonucleotide includes one or more expression sequences, and each of the one or more expression sequences is separated from a succeeding expression sequence by a stagger element on the circular or linear polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element includes a portion of an expression sequence of the one or more expression sequences.

Examples of stagger elements are described in paragraphs [0172]-[0175] of International Patent Publication No. WO2019/118919, which is hereby incorporated by reference in its entirety.

To avoid production of a continuous expression product while maintaining rolling circle translation, a stagger element may be included to induce ribosomal pausing during translation. In some embodiments, the stagger element is at 3′ end of at least one of the one or more expression sequences. The stagger element can be configured to stall a ribosome during rolling circle translation of the circular or linear polyribonucleotide. The stagger element may include, but is not limited to a 2A-like, or CHYSEL (SEQ ID NO: 191) (cis-acting hydrolase element) sequence. In some embodiments, the stagger element encodes a sequence with a C-terminal consensus sequence that is X₁X₂X₃EX₅NPGP, where X₁ is absent or G or H, X₂ is absent or D or G, X₃ is D or V or I or S or M, and X₅ is any amino acid (SEQ ID NO: 192). Some non-limiting examples of stagger elements includes GDVESNPGP (SEQ ID NO: 193), GDIEENPGP (SEQ ID NO: 194), VEPNPGP (SEQ ID NO: 195), IETNPGP (SEQ ID NO: 196), GDIESNPGP (SEQ ID NO: 197), GDVELNPGP (SEQ ID NO: 198), GDIETNPGP (SEQ ID NO: 199), GDVENPGP (SEQ ID NO: 200), GDVEENPGP (SEQ ID NO: 201), GDVEQNPGP (SEQ ID NO: 202), IESNPGP (SEQ ID NO: 203), GDIELNPGP (SEQ ID NO: 204), HDIETNPGP (SEQ ID NO: 205), HDVETNPGP (SEQ ID NO: 206), HDVEMNPGP (SEQ ID NO: 207), GDMESNPGP (SEQ ID NO: 208), GDVETNPGP (SEQ ID NO: 209), GDIEQNPGP (SEQ ID NO: 210), and DSEFNPGP (SEQ ID NO: 211).

In some embodiments, a stagger element described herein cleaves an expression product, such as between G and P of the consensus sequence described herein. As one non-limiting example, the circular or linear polyribonucleotide includes at least one stagger element to cleave the expression product. In some embodiments, the circular or linear polyribonucleotide includes a stagger element adjacent to at least one expression sequence. In some embodiments, the circular or linear polyribonucleotide includes a stagger element after each expression sequence. In some embodiments, the circular or linear polyribonucleotide includes a stagger element that is present on one or both sides of each expression sequence, leading to translation of individual peptide(s) and or polypeptide(s) from each expression sequence.

In some embodiments, a stagger element includes one or more modified nucleotides or unnatural nucleotides that induce ribosomal pausing during translation. Unnatural nucleotides may include peptide nucleic acid (PNA), Morpholino and locked nucleic acid (LNA), as well as glycol nucleic acid (GNA) and threose nucleic acid (TNA). Examples such as these are distinguished from naturally occurring DNA or RNA by changes to the backbone of the molecule. Exemplary modifications can include any modification to the sugar, the nucleobase, the internucleoside linkage (e.g., to a linking phosphate/to a phosphodiester linkage/to the phosphodiester backbone), and any combination thereof that can induce ribosomal pausing during translation. Some of the exemplary modifications provided herein are described elsewhere herein.

In some embodiments, a stagger element is present in a circular or linear polyribonucleotide in other forms. For example, in some exemplary circular or linear polyribonucleotides, a stagger element includes a termination element of a first expression sequence in the circular or linear polyribonucleotide, and a nucleotide spacer sequence that separates the termination element from a first translation initiation sequence of an expression succeeding the first expression sequence. In some examples, the first stagger element of the first expression sequence is upstream of (5′ to) a first translation initiation sequence of the expression succeeding the first expression sequence in the circular or linear polyribonucleotide. In some cases, the first expression sequence and the expression sequence succeeding the first expression sequence are two separate expression sequences in the circular or linear polyribonucleotide. The distance between the first stagger element and the first translation initiation sequence can enable continuous translation of the first expression sequence and its succeeding expression sequence. In some embodiments, the first stagger element includes a termination element and separates an expression product of the first expression sequence from an expression product of its succeeding expression sequences, thereby creating discrete expression products. In some cases, the circular or linear polyribonucleotide including the first stagger element upstream of the first translation initiation sequence of the succeeding sequence in the circular or linear polyribonucleotide is continuously translated, while a corresponding circular or linear polyribonucleotide including a stagger element of a second expression sequence that is upstream of a second translation initiation sequence of an expression sequence succeeding the second expression sequence is not continuously translated. In some cases, there is only one expression sequence in the circular or linear polyribonucleotide, and the first expression sequence and its succeeding expression sequence are the same expression sequence. In some exemplary circular or linear polyribonucleotides, a stagger element includes a first termination element of a first expression sequence in the circular or linear polyribonucleotide, and a nucleotide spacer sequence that separates the termination element from a downstream translation initiation sequence. In some such examples, the first stagger element is upstream of (5′ to) a first translation initiation sequence of the first expression sequence in the circular or linear polyribonucleotide. In some cases, the distance between the first stagger element and the first translation initiation sequence enables continuous translation of the first expression sequence and any succeeding expression sequences. In some embodiments, the first stagger element separates one round expression product of the first expression sequence from the next round expression product of the first expression sequences, thereby creating discrete expression products. In some cases, the circular or linear polyribonucleotide including the first stagger element upstream of the first translation initiation sequence of the first expression sequence in the circular or linear polyribonucleotide is continuously translated, while a corresponding circular or linear polyribonucleotide including a stagger element upstream of a second translation initiation sequence of a second expression sequence in the corresponding circular or linear polyribonucleotide is not continuously translated. In some cases, the distance between the second stagger element and the second translation initiation sequence is at least 2×, 3×, 4×, 5×, 6×, 7×, 8×, 9×, or 10× greater in the corresponding circular or linear polyribonucleotide than a distance between the first stagger element and the first translation initiation in the circular or linear polyribonucleotide. In some cases, the distance between the first stagger element and the first translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater. In some embodiments, the distance between the second stagger element and the second translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater than the distance between the first stagger element and the first translation initiation. In some embodiments, the circular or linear polyribonucleotide includes more than one expression sequence. In some embodiments, the plurality of expression sequences encoded by a circular ribonucleotide may be separated by an IRES between each expression sequence. For example, a circular polyribonucleotide may include a first IRES operable linked to a first expression sequence and a second IRES operably linked to a second expression sequence. The IRES may be the same IRES between all expression sequences. The IRES may be different between expression sequences.

In some embodiments, the plurality of expression sequences may be separated by a 2A self-cleaving peptide. For example, a circular polyribonucleotide may encode an IRES operably linked to an open reading frame encoding a first expression sequence, a 2A, and a second expression sequences. In some embodiments, the 2A may have a sequence of

(SEQ ID NO: 212)
GSGATNFSLLKQAGDVEENPGP.

In some embodiments, the plurality of expression sequences may be separated by a protease cleavage site (e.g., a furin cleavage site). For example, a circular polyribonucleotide may encode an IRES operably linked to an open reading frame encoding a first expression sequence, a protease cleavage site (e.g., a furin cleavage site), and a second expression sequence. In some embodiments, the furin cleavage site may have a sequence of GRLRR (SEQ ID NO: 213).

In some embodiments, the plurality of expression sequence may be separated by a 2A self-cleaving peptide and a protease cleavage site (e.g., a furin cleavage site). For example, a circular polyribonucleotide may encode an IRES operably linked to an open reading frame encoding a first expression sequence, a 2A, a protease cleavage site (e.g., a furin cleavage site), and a second expression sequence. A circular polyribonucleotide may also encode an IRES operably linked to an open reading frame encoding a first expression sequence, a protease cleavage site (e.g., a furin cleavage site), a 2A, and a second expression sequence. A tandem 2A and furin cleavage site may be referred to as a furin-2A (which includes furin-2A or 2A-furin, arranged in either orientation).

Furthermore, the plurality of expression sequences encoded by the circular ribonucleotide may be separated by both IRES and 2A sequences. For example, an IRES may be between one expression sequence and a second expression sequence while a 2A peptide may be between the second expression sequence and the third expression sequence. The selection of a particular IRES or 2A self-cleaving peptide may be used to control the expression level of expression sequence under control of the IRES or 2A sequence. For example, depending on the IRES and or 2A peptide selected, expression on the polypeptide may be higher or lower.

In some embodiments, a circular or linear polyribonucleotide includes at least one cleavage sequence. In some embodiments, the cleavage sequence is adjacent to an expression sequence. In some embodiments, the cleavage sequence is between two expression sequences. In some embodiments, cleavage sequence is included in an expression sequence. In some embodiments, the circular or linear polyribonucleotide includes between 2 and 10 cleavage sequences. In some embodiments, the circular or linear polyribonucleotide includes between 2 and 5 cleavage sequences. In some embodiments, the multiple cleavage sequences are between multiple expression sequences; for example, a circular or linear polyribonucleotide may include three expression sequences two cleavage sequences such that there is a cleavage sequence in between each expression sequence.

In some embodiments, the circular or linear polyribonucleotide includes a cleavage sequence, such as in an immolating circRNA or cleavable circRNA or self-cleaving circRNA. In some embodiments, the circular or linear polyribonucleotide includes two or more cleavage sequences, leading to separation of the circular or linear polyribonucleotide into multiple products, e.g., miRNAs, linear RNAs, smaller circular or linear polyribonucleotide, etc.

In some embodiments, a cleavage sequence includes a ribozyme RNA sequence. A ribozyme (from ribonucleic acid enzyme, also called RNA enzyme or catalytic RNA) is an RNA molecule that catalyzes a chemical reaction. Many natural ribozymes catalyze either the hydrolysis of one of their own phosphodiester bonds, or the hydrolysis of bonds in other RNA, but they have also been found to catalyze the aminotransferase activity of the ribosome. Catalytic RNA can be “evolved” by in vitro methods. Similar to riboswitch activity discussed above, ribozymes and their reaction products can regulate gene expression. In some embodiments, a catalytic RNA or ribozyme can be placed within a larger non-coding RNA such that the ribozyme is present at many copies within the cell for the purposes of chemical transformation of a molecule from a bulk volume. In some embodiments, aptamers and ribozymes can both be encoded in the same non-coding RNA.

In some embodiments, the cleavage sequence encodes a cleavable polypeptide linker. For example, a polyribonucleotide may encode two or more expression sequences are encoded by a single open-reading frame (ORF). For example, two or more expression sequences may be encoded by a single open-reading frame, the expression of which is controlled by an IRES. In some embodiments, the ORF further encodes a polypeptide linker, e.g., such that the expression product of the ORF encodes two or more expression sequences each separated by a sequence encoding a polypeptide linker (e.g., a linker of 5-200, 5 to 100, 5 to 50, 5 to 20, 50 to 100, or 50 to 200 amino acids). The polypeptide linker may include a cleavage site, for example, a cleavage site recognized and cleaved by a protease (e.g., an endogenous protease in a subject following administration of the polyribonucleotide to that subject). In such embodiments, a single expression product including the amino acid sequence of two or more expression sequences is cleaved upon expression, such that the two or more expression sequences are separated following expression. Exemplary protease cleavage sites are known to those of skill in the art, for example, amino acid sequences that act as protease cleavage sites recognized by a metalloproteinase (e.g., a matrix metalloproteinase (MMP), such as any one or more of MMPs 1-28), a disintegrin and metalloproteinase (ADAM, such as any one or more of ADAMs 2, 7-12, 15, 17-23, 28-30 and 33), a serine protease, urokinase-type plasminogen activator, matriptase, a cysteine protease, an aspartic protease, or a cathepsin protease. In some embodiments, the protease is MMP9 or MMP2. In some embodiments, the protease is matriptase.

In some embodiments, a circular or linear polyribonucleotide described herein is an immolating circular or linear polyribonucleotide, a cleavable circular or linear polyribonucleotide, or a self-cleaving circular or linear polyribonucleotide. A circular or linear polyribonucleotide can deliver cellular components including, for example, RNA, lncRNA, lincRNA, miRNA, tRNA, rRNA, snoRNA, ncRNA, SIRNA, or shRNA. In some embodiments, a circular or linear polyribonucleotide includes miRNA separated by (i) self-cleavable elements; (ii) cleavage recruitment sites; (iii) degradable linkers; (iv) chemical linkers; and/or (v) spacer element sequences. In some embodiments, circRNA includes siRNA separated by (i) self-cleavable elements; (ii) cleavage recruitment sites (e.g., ADAR); (iii) degradable linkers (e.g., glycerol); (iv) chemical linkers; and/or (v) spacer element sequences. Non-limiting examples of self-cleavable elements include hammerhead, splicing element, hairpin, hepatitis delta virus (HDV), Varkud Satellite (VS), and glmS ribozymes.

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and/or polypeptide(s). In some embodiments, the stagger element is a portion of the one or more expression sequences. In some embodiments, the circular polyribonucleotide comprises one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element comprises a portion of an expression sequence of the one or more expression sequences.

### Translation Initiation Sequences

In some embodiments, a circular or linear polyribonucleotide encodes an expression sequence and includes a translation initiation sequence, e.g., a start codon. In some embodiments, the polyribonucleotide includes a translation initiation sequence operably linked to an expression sequence. In some embodiments, the translation initiation sequence includes a Kozak or Shine-Dalgarno sequence. In some embodiments, the translation initiation sequence includes a Kozak sequence. In some embodiments, the circular or linear polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the circular or linear polyribonucleotide includes at least one translation initiation sequence adjacent to an expression sequence. In some embodiments, the translation initiation sequence provides conformational flexibility to the circular or linear polyribonucleotide. In some embodiments, the translation initiation sequence is within a single stranded region of the circular or linear polyribonucleotide. Further examples of translation initiation sequences are described in paragraphs [0163]-[0165] of International Patent Publication No. WO2019/118919, which is hereby incorporated by reference in its entirety.

The circular or linear polyribonucleotide may include more than one start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, a circular or linear polyribonucleotide may initiate at a codon which is not the first start codon, e.g., AUG. Translation of the circular or linear polyribonucleotide may initiate at an alternative translation initiation sequence, such as, but not limited to, ACG, AGG, AAG, CTG/CUG, GTG/GUG, ATA/AUA, ATT/AUU, TTG/UUG. In some embodiments, translation begins at an alternative translation initiation sequence under selective conditions, e.g., stress induced conditions. As a non-limiting example, the translation of the polyribonucleotide may begin at alternative translation initiation sequence, such as ACG. As another non-limiting example, the polyribonucleotide translation may begin at alternative translation initiation sequence, CTG/CUG. As another non-limiting example, the polyribonucleotide translation may begin at alternative translation initiation sequence, GTG/GUG. As another non-limiting example, the polyribonucleotide may begin translation at a repeat-associated non-AUG (RAN) sequence, such as an alternative translation initiation sequence that includes short stretches of repetitive RNA e.g., CGG, GGGGCC, CAG, CTG.

In some embodiments, translation is initiated by eukaryotic initiation factor 4A (elF4A) treatment with Rocaglates (translation is repressed by blocking 43S scanning, leading to premature, upstream translation initiation and reduced protein expression from transcripts bearing the RocA-elF4A target sequence, see for example, nature.com/articles/nature17978).

### Termination Elements

In some embodiments, the polyribonucleotide described herein (e.g., the polyribonucleotide cargo of the polyribonucleotide) includes least one termination element. In some embodiments, the polyribonucleotide includes a termination element operably linked to an expression sequence. In some embodiments, the polynucleotide lacks a termination element.

In some embodiments, the polyribonucleotide includes one or more expression sequences, and each expression sequence may or may not have a termination element. In some embodiments, the polyribonucleotide includes one or more expression sequences, and the expression sequences lack a termination element, such that the polyribonucleotide is continuously translated. Exclusion of a termination element may result in rolling circle translation or continuous expression of expression product.

In some embodiments, the circular polyribonucleotide includes one or more expression sequences, and each expression sequence may or may not have a termination element. In some embodiments, the circular polyribonucleotide includes one or more expression sequences, and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide includes a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences includes two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation, e.g., UAA, UGA, UAG. In some embodiments, one or more termination elements in the circular polyribonucleotide are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell. In some embodiments, the termination element is a stop codon.

Further examples of termination elements are described in paragraphs [0169]-[0170] of International Patent Publication No. WO2019/118919, which is hereby incorporated by reference in its entirety.

### Polyribonucleotide Cargo

In some embodiments, the circular polyribonucleotide encodes a polyribonucleotide cargo. A polyribonucleotide cargo described herein includes any sequence including at least one polyribonucleotide. In some embodiments, the polyribonucleotide cargo includes an expression sequence, a non-coding sequence, or an expression sequence and a non-coding sequence. In some embodiments, the polyribonucleotide cargo includes an expression sequence encoding a polypeptide. In some embodiments, the polyribonucleotide cargo includes an IRES operably linked to an expression sequence encoding a polypeptide. In some embodiments, the polyribonucleotide cargo includes an expression sequence that encodes a polypeptide that has a biological effect on a subject.

A polyribonucleotide cargo may, for example, include at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the polyribonucleotides cargo includes from 1-20,000 nucleotides, 1-10,000 nucleotides, 1-5,000 nucleotides, 100-20,000 nucleotide, 100-10,000 nucleotides, 100-5,000 nucleotides, 500-20,000 nucleotides, 500-10,000 nucleotides, 500-5,000 nucleotides, 1,000-20,000 nucleotides, 1,000-10,000 nucleotides, or 1,000-5,000 nucleotides.

In embodiments, the polyribonucleotide cargo includes one or multiple expression (or coding) sequences, wherein each expression (or coding) sequence encodes a polypeptide. In embodiments, the polyribonucleotide cargo includes one or multiple noncoding sequences. In embodiments, the polyribonucleotide cargo consists entirely of non-coding sequence(s). In embodiments, the polyribonucleotide cargo includes a combination of expression (or coding) and noncoding sequences.

In some embodiments, the GC content of a nucleic acid sequence encoding a polypeptide is at least 51% (e.g., at least 52%, 53%, 54%, 55%, 56%, 57%, 58%, 59%, or 60%). In some embodiments, the GC content of a nucleic acid sequence encoding a polypeptide is at most 52%, 53%, 54%, 55%, 56%, 57%, 58% or 59%, or 60%. In some embodiments, the GC content of a nucleic acid sequence encoding a polypeptide is 51% to 60%, 52% to 60%, 53% to 60%, 54% to 60%, 55% to 60%, 52% to 58%, 53% to 58%.

In some embodiments, the uridine content (for RNA) or the thymidine content (for DNA) of a nucleic acid sequence encoding polypeptide is more than 10% (e.g., more than 11%, 12%, 13%, 14%, 15%, 16%, 17%, 18%, 19%, 20%, 21%, 22%, 23%, 24%, or 25%). In some embodiments, the uridine content (for RNA) or the thymidine content (for DNA) of a nucleic acid sequence encoding a polypeptide is at most 30% (e.g., at most 29%, 28%, 27%, 26%, 25%, 24%, 23%, 22%, 21%, or 20%). In some embodiments, the uridine content (for RNA) or the thymidine content (for DNA) of a nucleic acid sequence encoding a polypeptide is 20% to 28%, 21% to 26%, 10% to 24%, 15% to 24%, 20% to 24%, 21% to 24%, 22% to 24%, 23% to 24%, 10% to 23%, 15% to 23%, 20% to 23%, 21% to 23%, or 22% to 23%.

The GC content of an expression sequence encoding the polypeptide refers to the GC content of the expression sequence that exclusively encodes the polypeptide with no other coding regions that encode peptides other than the polypeptide. Likewise, the uridine content or thymidine of an expression sequence encoding the polypeptide refers to the uridine content of the expression sequence that exclusively encodes the polypeptide with no other coding regions that encode peptides other than the polypeptide. In some embodiments, the calculation of the GC content or the uridine (or thymidine) content of the expression sequence encoding the polypeptide only takes into account the continuous nucleic acid sequence that starts in a 5′ to 3′ direction from the first nucleoside of the start codon of the open reading frame that encodes the polypeptide to the last nucleoside of the stop codon of the same open reading frame. In other embodiments, the calculation of the GC content or the uridine (or thymidine) content of the expression sequence encoding the polypeptide only takes into account the continuous nucleic acid sequence that starts in a 5′ to 3′ direction from the first nucleoside of the codon that encodes the N-terminal end amino acid residue of the polypeptide to the last nucleoside of the codon that encodes the C-terminal end amino acid residue of the polypeptide.

In some embodiments, the nucleic acid sequence encoding the polypeptide has a uridine content of more than 20%. In some embodiments, the uridine content of a nucleic acid sequence encoding polypeptide is more than 10% (e.g., more than 11%, 12%, 13%, 14%, 15%, 16%, 17%, 18%, 19%, 20%, 21%, 22%, 23%, 24%, or 25%). In some embodiments, the uridine content of a nucleic acid sequence encoding polypeptide is at most 30% (e.g., at most 29%, 28%, 27%, 26%, 25%, 24%, 23%, 22%, 21%, or 20%). In some embodiments, the uridine content of a nucleic acid sequence encoding polypeptide is 20% to 28%, 21% to 26%, 10% to 24%, 15% to 24%, 20% to 24%, 21% to 24%, 22% to 24%, 23% to 24%, 10% to 23%, 15% to 23%, 20% to 23%, 21% to 23%, or 22% to 23%. In some embodiments, the nucleic acid sequence encoding the polypeptide has a uridine content of 20% to 28%.

In some embodiments, polyribonucleotides made as described herein are used as effectors in therapy or agriculture. For example, a circular polyribonucleotide made by the methods described herein (e.g., the cell-free methods described herein) may be administered to a subject (e.g., in a pharmaceutical, or agricultural composition). In another example, a circular polyribonucleotide made by the methods described herein (e.g., the cell-free methods described herein) may be delivered to a cell.

In some embodiments, the polyribonucleotide includes any feature, or any combination of features as disclosed in International Patent Publication No. WO2019/118919, which is hereby incorporated by reference in its entirety.

### Polypeptide Expression Sequences

In some embodiments, the polyribonucleotide described herein (e.g., the polyribonucleotide cargo of the circular polyribonucleotide) includes one or more expression (or coding) sequences, wherein each expression sequence encodes a polypeptide. In some embodiments, the circular polyribonucleotide includes two, three, four, five, six, seven, eight, nine, ten or more expression (or coding) sequences.

Each encoded polypeptide may be linear or branched. In various embodiments, the polypeptide has a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful.

Polypeptides included herein may include naturally occurring polypeptides or non-naturally occurring polypeptides. In some embodiments, the polypeptide is or includes a functional fragment or variant of a reference polypeptide (e.g., an enzymatically active fragment or variant of an enzyme). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a protein of interest.

Some examples of a polypeptide include, but are not limited to, a fluorescent tag or marker, an antigen, a therapeutic polypeptide, or a polypeptide for agricultural applications.

A therapeutic polypeptide may be a hormone, a neurotransmitter, a growth factor, an enzyme (e.g., oxidoreductase, metabolic enzyme, mitochondrial enzyme, oxygenase, dehydrogenase, ATP-independent enzyme, lysosomal enzyme, desaturase), a cytokine, an antigen binding polypeptide (e.g., antigen binding antibody or antibody-like fragments, such as single chain antibodies, nanobodies or other Ig heavy chain or light chain containing polypeptides), an Fc fusion protein, an anticoagulant, a blood factor, a bone morphogenetic protein, an interferon, an interleukin, and a thrombolytic.

A polypeptide for agricultural applications may be a bacteriocin, a lysin, an antimicrobial polypeptide, an antifungal polypeptide, a nodule C-rich peptide, a bacteriocyte regulatory peptide, a peptide toxin, a pesticidal polypeptide (e.g., insecticidal polypeptide or nematocidal polypeptide), an antigen binding polypeptide (e.g., antigen binding antibody or antibody-like fragments, such as single chain antibodies, nanobodies or other Ig heavy chain or light chain containing polypeptides), an enzyme (e.g., nuclease, amylase, cellulase, peptidase, lipase, chitinase), a peptide pheromone, and a transcription factor.

In some embodiments, the circular polyribonucleotide expresses a non-human protein.

In some embodiments, the circular polyribonucleotide expresses an antibody, e.g., an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can include more than one expression (or coding) sequence, each of which expresses a portion of an antibody, and the sum of which can constitute the antibody. In some cases, the circular polyribonucleotide includes one expression sequence coding for the heavy chain of an antibody, and another expression sequence coding for the light chain of the antibody. In some cases, when the circular polyribonucleotide is expressed in a cell or a cell-free environment, the light chain and heavy chain can be subject to appropriate modification, folding, or other post-translation modification to form a functional antibody.

In embodiments, polypeptides include multiple polypeptides, e.g., multiple copies of one polypeptide sequence, or multiple different polypeptide sequences. In embodiments, multiple polypeptides are connected by linker amino acids or spacer amino acids.

In embodiments, the polynucleotide cargo includes a sequence encoding a signal peptide. Many signal peptide sequences have been described, for example, the Tat (Twin-arginine translocation) signal sequence is typically an N-terminal peptide sequence containing a consensus SRRxFLK “twin-arginine” motif, which serves to translocate a folded protein containing such a Tat signal peptide across a lipid bilayer. See also, e.g., the Signal Peptide Database publicly available at www[dot]signalpeptide[dot]de. Signal peptides are also useful for directing a protein to specific organelles; see, e.g., the experimentally determined and computationally predicted signal peptides disclosed in the Spdb signal peptide database, publicly available at proline,bic.nus.edu.sg/spdb.

In embodiments, the polynucleotide cargo includes sequence encoding a cell-penetrating peptide (CPP). Hundreds of CPP sequences have been described; see, e.g., the database of cell-penetrating peptides, CPPsite, publicly available at crdd[dot]osdd[dot]net/raghava/cppsite/. An example of a commonly used CPP sequence is a poly-arginine sequence, e.g., octaarginine or nonoarginine, which can be fused to the C-terminus of the CGI peptide.

In embodiments, the polynucleotide cargo includes sequence encoding a self-assembling peptide; see, e.g., Miki et al. (2021) Nature Communications, 21:3412, DOI: 10.1038/s41467-021-23794-6.

In some embodiments, the expression (or coding) sequence includes a poly-A sequence (e.g., at the 3′ end of an expression sequence). In some embodiments, the length of a poly-A sequence is greater than 10 nucleotides in length. In one embodiment, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is designed according to the descriptions of the poly-A sequence in [0202]-[0204] of International Patent Publication No. WO2019/118919A1, which is incorporated herein by reference in its entirety. In some embodiments, the expression sequence lacks a poly-A sequence (e.g., at the 3′ end of an expression sequence).

In some embodiments, a circular polyribonucleotide includes a polyA, lacks a polyA, or has a modified polyA to modulate one or more characteristics of the circular polyribonucleotide. In some embodiments, the circular polyribonucleotide lacking a polyA or having modified polyA improves one or more functional characteristics, e.g., immunogenicity (e.g., the level of one or more marker of an immune or inflammatory response), half-life, and/or expression efficiency.

### Therapeutic Polypeptides

In some embodiments, the circular polyribonucleotide described herein (e.g., the polyribonucleotide cargo of the circular polyribonucleotide) includes at least one expression sequence encoding a therapeutic polypeptide. A therapeutic polypeptide is a polypeptide that when administered to or expressed in a subject provides some therapeutic benefit. Administration to a subject or expression in a subject of a therapeutic polypeptide may be used to treat or prevent a disease, disorder, or condition or a symptom thereof. In some embodiments, the circular polyribonucleotide encodes two, three, four, five, six, seven, eight, nine, ten or more therapeutic polypeptides.

In some embodiments, the circular polyribonucleotide includes an expression sequence encoding a therapeutic protein. The protein may treat the disease in the subject in need thereof. In some embodiments, the therapeutic protein can compensate for a mutated, under-expressed, or absent protein in the subject in need thereof. In some embodiments, the therapeutic protein can target, interact with, or bind to a cell, tissue, or virus in the subject in need thereof.

A therapeutic polypeptide can be a polypeptide that can be secreted from a cell, or localized to the cytoplasm, nucleus, or membrane compartment of a cell.

A therapeutic polypeptide may be a hormone, a neurotransmitter, a growth factor, an enzyme (e.g., oxidoreductase, metabolic enzyme, mitochondrial enzyme, oxygenase, dehydrogenase, ATP-independent enzyme, lysosomal enzyme, desaturase), a cytokine, a transcription factor, an antigen binding polypeptide (e.g., antigen binding antibody or antibody-like fragments, such as single chain antibodies, nanobodies or other Ig heavy chain or light chain containing polypeptides), an Fc fusion protein, an anticoagulant, a blood factor, a bone morphogenetic protein, an interferon, an interleukin, a thrombolytic, an antigen (e.g., a tumor, viral, or bacterial antigen), a nuclease (e.g., an endonuclease such as a Cas protein, e.g., Cas9), a membrane protein (e.g., a chimeric antigen receptor (CAR), a transmembrane receptor, a G-protein-coupled receptor (GPCR), a receptor tyrosine kinase (RTK), an antigen receptor, an ion channel, or a membrane transporter), a secreted protein, a gene editing protein (e.g., a CRISPR-Cas, TALEN, or zinc finger), or a gene writing protein (see, e.g., International Patent Publication No. WO2020/047124, incorporated in its entirety herein by reference).

In some embodiments, the therapeutic polypeptide is an antibody, e.g., a full-length antibody, an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can include more than one expression sequence, each of which expresses a portion of an antibody, and the sum of which can constitute the antibody. In some cases, the circular polyribonucleotide includes one expression sequence coding for the heavy chain of an antibody, and another expression sequence coding for the light chain of the antibody. When the circular polyribonucleotide is expressed in a cell, the light chain and heavy chain can be subject to appropriate modification, folding, or other post-translation modification to form a functional antibody.

In some embodiments, circular polyribonucleotides made as described herein are used as effectors in therapy or agriculture. For example, a circular polyribonucleotide made by the methods described herein (e.g., the cell-free methods described herein) may be administered to a subject (e.g., in a pharmaceutical or agricultural composition). In embodiments, the subject is a vertebrate animal (e.g., mammal, bird, fish, reptile, or amphibian). In embodiments, the subject is a human. In embodiments, the method subject is a non-human mammal. In embodiments, the subject is a non-human mammal such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusc. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a geophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

### Secreted Polypeptide Effectors

In some embodiments, the circular polyribonucleotide described herein (e.g., the polyribonucleotide cargo of the circular polyribonucleotide) includes at least one coding sequence encoding a secreted polypeptide effector. Exemplary secreted polypeptide effectors or proteins that may be expressed include, e.g., cytokines and cytokine receptors, polypeptide hormones and receptors, growth factors, clotting factors, therapeutic replacement enzymes and therapeutic non-enzymatic effectors, regeneration, repair, and fibrosis factors, transformation factors, and proteins that stimulate cellular regeneration, non-limiting examples of which are described herein, e.g., in the tables below.

### Cytokines and Cytokine Receptors

In some embodiments, an effector described herein comprises a cytokine of Table 2, or a functional variant or fragment thereof, e.g., a protein having at least 80%, 85%, 90%, 95%, 967%, 98%, 99% identity to a protein sequence disclosed in Table 11 by reference to its UniProt ID. In some embodiments, the functional variant binds to the corresponding cytokine receptor with a Kd of no more than 10%, 20%, 30%, 40%, or 50% higher or lower than the Kd of the corresponding wild-type cytokine for the same receptor under the same conditions. In some embodiments, the effector comprises a fusion protein comprising a first region (e.g., a cytokine polypeptide of Table 1 or a functional variant or fragment thereof) and a second, heterologous region. In some embodiments, the first region is a first cytokine polypeptide of Table 11. In some embodiments, the second region is a second cytokine polypeptide of Table 11, wherein the first and second cytokine polypeptides form a cytokine heterodimer with each other in a wild-type cell. In some embodiments, the polypeptide of Table 11 or functional variant thereof comprises a signal sequence, e.g., a signal sequence that is endogenous to the effector, or a heterologous signal sequence.

In some embodiments, an effector described herein comprises an antibody or fragment thereof that binds a cytokine of Table 11. In some embodiments, the antibody molecule comprises a signal sequence.

TABLE 11
Exemplary cytokines and cytokine receptors

| Cytokine | Cytokine receptor(s) | Entrez Gene ID¹ | UniProt ID² |
|---|---|---|---|
| IL-1α, IL-1β, or a | IL-1 type 1 receptor, IL-1 type | 3552, 3553 | P01583, P01584 |
| heterodimer thereof | 2 receptor | | |
| IL-1Ra | IL-1 type 1 receptor, IL-1 type | 3454, 3455 | P17181, P48551 |
| | 2 receptor | | |
| IL-2 | IL-2R | 3558 | P60568 |
| IL-3 | IL-3 receptor α + β c (CD131) | 3562 | P08700 |
| IL-4 | IL-4R type I, IL-4R type II | 3565 | P05112 |
| IL-5 | IL-5R | 3567 | P05113 |
| IL-6 | IL-6R (sIL-6R) gp130 | 3569 | P05231 |
| IL-7 | IL-7R and sIL-7R | 3574 | P13232 |
| IL-8 | CXCR1 and CXCR2 | 3576 | P10145 |
| IL-9 | IL-9R | 3578 | P15248 |
| IL-10 | IL-10R1/IL-10R2 complex | 3586 | P22301 |
| IL-11 | IL-11Rα 1 gp130 | 3589 | P20809 |
| IL-12 (e.g., p35, p40, or a | IL-12Rβ1 and IL-12Rβ2 | 3593, 3592 | P29459, P29460 |
| heterodimer thereof) | | | |
| IL-13 | IL-13R1α1 and IL-13R1α2 | 3596 | P35225 |
| IL-14 | IL-14R | 30685 | P40222 |
| IL-15 | IL-15R | 3600 | P40933 |
| IL-16 | CD4 | 3603 | Q14005 |
| IL-17A | IL-17RA | 3605 | Q16552 |
| IL-17B | IL-17RB | 27190 | Q9UHF5 |
| IL-17C | IL-17RA to IL-17RE | 27189 | Q9P0M4 |
| IL-17D | SEF | 53342 | Q8TAD2 |
| IL-17F | IL-17RA, IL-17RC | 112744 | Q96PD4 |
| IL-18 | IL-18 receptor | 3606 | Q14116 |
| IL-19 | IL-20R1/IL-20R2 | 29949 | Q9UHD0 |
| IL-20 | L-20R1/IL-20R2 and | 50604 | Q9NYY1 |
| | IL-22R1/IL-20R2 | | |
| IL-21 | IL-21R | 59067 | Q9HBE4 |
| IL-22 | IL-22R | 50616 | Q9GZX6 |
| IL-23 (e.g., p19, p40, or a | IL-23R | 51561 | Q9NPF7 |
| heterodimer thereof) | | | |
| IL-24 | IL-20R1/IL-20R2 and | 11009 | Q13007 |
| | IL-22R1/IL-20R2 | | |
| IL-25 | IL-17RA and IL-17RB | 64806 | Q9H293 |
| IL-26 | IL-10R2 chain and | 55801 | Q9NPH9 |
| | IL-20R1 chain | | |
| IL-27 (e.g., p28, EBI3, or | WSX-1 and gp130 | 246778 | Q8NEV9 |
| a heterodimer thereof) | | | |
| IL-28A, IL-28B, and IL29 | IL-28R1/IL-10R2 | 282617, 282618 | Q8IZI9, Q8IU54 |
| IL-30 | IL6R/gp130 | 246778 | Q8NEV9 |
| IL-31 | IL-31RA/OSMRβ | 386653 | Q6EBC2 |
| IL-32 | | 9235 | P24001 |
| IL-33 | ST2 | 90865 | O95760 |
| IL-34 | Colony-stimulating | 146433 | Q6ZMJ4 |
| | factor 1 receptor | | |
| IL-35 (e.g., p35, EBI3, or | IL-12Rβ2/gp130; | 10148 | Q14213 |
| a heterodimer thereof) | IL-12Rβ2/IL-12Rβ2; | | |
| | gp130/gp130 | | |
| IL-36 | IL-36Ra | 27179 | Q9UHA7 |
| IL-37 | IL-18Rα and IL-18BP | 27178 | Q9NZH6 |
| IL-38 | IL-1R1, IL-36R | 84639 | Q8WWZ1 |
| IFN-α | IFNAR | 3454 | P17181 |
| IFN-β | IFNAR | 3454 | P17181 |
| IFN-γ | IFNGR1/IFNGR2 | 3459 | P15260 |
| TGF-β | TβR-I and TβR-II | 7046, 7048 | P36897, P37173 |
| TNF-α | TNFR1, TNFR2 | 7132, 7133 | P19438, P20333 |

¹Sequence available on the NCBI database on the world wide web internet site ncbi.nlm.nih.gov/gene; Maglott D, et al. Gene: a gene-centered information resource at NCBI. Nucleic Acids Res. 2014. pii: gku1055.
²Sequence available on the Uniprot database on the world wide web internet site uniprot.org/uniprot; UniProt: the universal protein knowledgebase in 2021. Nucleic Acids Res. 49: D1 (2021).

### Polypeptide Hormones and Receptors

In some embodiments, an effector described herein comprises a hormone of Table 12, or a functional variant thereof, e.g., a protein having at least 80%, 85%, 90%, 95%, 967%, 98%, 99% identity to a protein sequence disclosed in Table 12 by reference to its UniProt ID. In some embodiments, the functional variant binds to the corresponding receptor with a Kd of no more than 10%, 20%, 30%, 40%, or 50% higher than the Kd of the corresponding wild-type hormone for the same receptor under the same conditions. In some embodiments, the polypeptide of Table 12 or functional variant thereof comprises a signal sequence, e.g., a signal sequence that is endogenous to the effector, or a heterologous signal sequence.

In some embodiments, an effector described herein comprises an antibody molecule (e.g., an scFv) that binds a hormone of Table 12. In some embodiments, an effector described herein comprises an antibody molecule (e.g., an scFv) that binds a hormone receptor of Table 12. In some embodiments, the antibody molecule comprises a signal sequence.

TABLE 12
Exemplary polypeptide hormones and receptors

| | | Entrez | |
|---|---|---|---|
| | | Gene | UniProt |
| Hormone | Receptor | ID¹ | ID² |
| Natriuretic Peptide, e.g., | NPRA, NPRB, | 4878 | P01160 |
| Atrial Natriuretic Peptide | NPRC | | |
| (ANP) | | | |
| Brain Natriuretic Peptide | NPRA, NPRB | 4879 | P16860 |
| (BNP) | | | |
| C-type natriuretic peptide | NPRB | 4880 | P23582 |
| (CNP) | | | |
| Growth hormone (GH) | GHR | 2690 | P10912 |
| Prolactin (PRL) | PRLR | 5617 | P01236 |
| Thyroid-stimulating | TSH receptor | 7253 | P16473 |
| hormone (TSH) | | | |
| Adrenocorticotropic | ACTH receptor | 5443 | P01189 |
| hormone (ACTH) | | | |
| Follicle-stimulating | FSHR | 2492 | P23945 |
| hormone (FSH) | | | |
| Luteinizing hormone (LH) | LHR | 3973 | P22888 |
| Antidiuretic hormone (ADH) | Vasopressin | 554 | P30518 |
| | receptors, e.g., | | |
| | V2; AVPR1A; | | |
| | AVPR1B; AVPR3; | | |
| | AVPR2 | | |
| Oxytocin | OXTR | 5020 | P01178 |
| Calcitonin | Calcitonin receptor | 796 | P01258 |
| | (CT) | | |
| Parathyroid hormone (PTH) | PTH1R and PTH2R | 5741 | P01270 |
| Insulin | Insulin receptor (IR) | 3630 | P01308 |
| Glucagon | Glucagon receptor | 2641 | P01275 |
| GIP | GIPR | 2695 | P09681 |
| Fibroblast growth | FGFR4 | 9965 | O95750 |
| factor 19 (FGF19) | | | |
| Fibroblast growth | FGFR1c, 2c, 3c | 26291 | Q9NSA1 |
| factor 21 (FGF21) | | | |
| Fibroblast growth | FGFR1, 2, 4 | 8074 | Q9GZV9 |
| factor 23 (FGF23) | | | |
| Melanocyte-stimulating | MC1R, MC4R, | | |
| hormone (alpha- MSH) | MC5R | | |
| Melanocyte-stimulating | MC4R | | |
| hormone (beta- MSH) | | | |
| Melanocyte-stimulating | MC1R, MC3R, | | |
| hormone (gamma- MSH) | MC4R, MC5R | | |
| Proopiomelanocortin | MC1R, MC3R, | 5443 | P01189 |
| POMC (alpha- beta-, | MC4R, MC5R | | |
| gamma-, MSH precursor) | | | |
| Glycoprotein hormones | | 1081 | P01215 |
| alpha chain (CGA) | | | |
| Follicle-stimulating | FSHR | 2488 | P01225 |
| hormone beta (FSHB) | | | |
| Leptin | LEPR | 3952 | P41159 |
| Ghrelin | GHSR | 51738 | Q9UBU3 |

¹Sequence available on the NCBI database on the world wide web internet site “ncbi.nlm.nih.gov/gene” (Maglott D, et al. Gene: a gene-centered information resource at NCBI. Nucleic Acids Res. 2014. pii: gku1055).
²Sequence available on the Uniprot database on the world wide web internet site “uniprot.org/uniprot/”; UniProt: the universal protein knowledgebase in 2021. Nucleic Acids Res. 49: D1 (2021).

### Growth Factors

In some embodiments, an effector described herein comprises a growth factor of Table 13, or a functional variant thereof, e.g., a protein having at least 80%, 85%, 90%, 95%, 967%, 98%, 99% identity to a protein sequence disclosed in Table 13 by reference to its UniProt ID. In some embodiments, the functional variant binds to the corresponding receptor with a Kd of no more than 10%, 20%, 30%, 40%, or 50% higher than the Kd of the corresponding wild-type growth factor for the same receptor under the same conditions. In some embodiments, the polypeptide of Table 13 or functional variant thereof comprises a signal sequence, e.g., a signal sequence that is endogenous to the effector, or a heterologous signal sequence.

In some embodiments, an effector described herein comprises an antibody or fragment thereof that binds a growth factor of Table 13. In some embodiments, an effector described herein comprises an antibody molecule (e.g., an scFv) that binds a growth factor receptor of Table 13. In some embodiments, the antibody molecule comprises a signal sequence.

TABLE 13
Exemplary growth factors

| | | Entrez Gene ID¹ | UniProt ID² |
|---|---|---|---|
| PDGF family | | | |
| PDGF (e.g., PDGF-1, | PDGF receptor, | 5156 | P16234 |
| PDGF-2, or a | e.g., PDGFRα, | | |
| heterodimer thereof) | PDGFRβ | | |
| CSF-1 | CSF1R | 1435 | P09603 |
| SCF | CD117 | 3815 | P10721 |
| VEGF family | | | |
| VEGF (e.g., isoforms | VEGFR-1, | 2321 | P17948 |
| VEGF 121, VEGF | VEGFR-2 | | |
| 165, VEGF 189, and | | | |
| VEGF 206) | | | |
| VEGF-B | VEGFR-1 | 2321 | P17949 |
| VEGF-C | VEGFR-2 and | 2324 | P35916 |
| | VEGFR -3 | | |
| PIGF | VEGFR-1 | 5281 | Q07326 |
| EGF family | | | |
| EGF | EGFR | 1950 | P01133 |
| TGF-α | EGFR | 7039 | P01135 |
| amphiregulin | EGFR | 374 | P15514 |
| HB-EGF | EGFR | 1839 | Q99075 |
| betacellulin | EGFR, ErbB-4 | 685 | P35070 |
| epiregulin | EGFR, ErbB-4 | 2069 | O14944 |
| Heregulin | EGFR, ErbB-4 | 3084 | Q02297 |
| FGF family | | | |
| FGF-1, FGF-2, FGF-3, | FGFR1, FGFR2, | 2246, 2247, 2248, | P05230, P09038, |
| FGF-4, FGF-5, FGF-6, | FGFR3, and | 2249, 2250, 2251, | P11487, P08620, |
| FGF-7, FGF-8, FGF-9 | FGFR4 | 2252, 2253, 2254 | P12034, P10767, |
| | | | P21781, P55075, |
| | | | P31371 |
| Insulin family | | | |
| Insulin | IR | 3630 | P01308 |
| IGF-I | IGF-I receptor, IGF- | 3479 | P05019 |
| | II receptor | | |
| IGF-II | IGF-II receptor | 3481 | P01344 |
| HGF family | | | |
| HGF | MET receptor | 3082 | P14210 |
| MSP | RON | 4485 | P26927 |
| Neurotrophin family | | | |
| NGF | LNGFR, trkA | 4803 | P01138 |
| BDNF | trkB | 627 | P23560 |
| NT-3 | trkA, trkB, trkC | 4908 | P20783 |
| NT-4 | trkA, trkB | 4909 | P34130 |
| NT-5 | trkA, trkB | 4909 | P34130 |
| Angiopoietin family | | | |
| ANGPT1 | HPK-6/TEK | 284 | Q15389 |
| ANGPT2 | HPK-6/TEK | 285 | O15123 |
| ANGPT3 | HPK-6/TEK | 9068 | O95841 |
| ANGPT4 | HPK-6/TEK | 51378 | Q9Y264 |
| ANGPTL2 | LILRB2 & integrin | 23452 | Q9UKU9 |
| | α5β1 | | |
| ANGPTL3 | LPL | 27329 | Q9Y5C1 |
| ANGPTL4 | | 51129 | Q9BY76 |
| ANGPTL8 | PirB | 55908 | Q6UXH0 |

¹Sequence available on the NCBI database on the world wide web internet site ncbi.nlm.nih.gov/gene.
²Sequence available on the Uniprot database on the world wide web internet site uniprot.org/uniport.

### Clotting Factors

In some embodiments, an effector described herein comprises a polypeptide of Table 14, or a functional variant thereof, e.g., a protein having at least 80%, 85%, 90%, 95%, 967%, 98%, 99% identity to a protein sequence disclosed in Table 14 by reference to its UniProt ID. In some embodiments, the functional variant catalyzes the same reaction as the corresponding wild-type protein, e.g., at a rate no less than 10%, 20%, 30%, 40%, or 50% lower or higher than the wild-type protein. In some embodiments, the polypeptide of Table 14 or functional variant thereof comprises a signal sequence, e.g., a signal sequence that is endogenous to the effector, or a heterologous signal sequence.

TABLE 14
Clotting-associated factors

| | | Entrez | |
|---|---|---|---|
| | | Gene | UniProt |
| Effector | Indication | ID¹ | ID² |
| Factor I | Afibrinogenomia | 2243, | P02671, |
| (fibrinogen) | | 2266, | P02679, |
| | | 2244 | P02675 |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor Deficiency | 2159 | P00742 |
| Factor XI | Hemophilia C | 2160 | P03951 |
| Factor XIII | Fibrin Stabilizing factor | 2162, | P00488, |
| | deficiency | 2165 | P05160 |
| vWF | von Willebrand disease | 7450 | P04275 |

¹Sequence available on the NCBI database on the world wide web internet site ncbi.nlm.nih.gov/gene.
²Sequence available on the Uniprot database on the world wide web internet site “uniprot.org/uniprot.

### Therapeutic Replacement Enzymes

In some embodiments, an effector described herein comprises an enzyme of Table 15, or a functional variant thereof, e.g., a protein having at least 80%, 85%, 90%, 95%, 967%, 98%, 99% identity to a protein sequence disclosed in Table 15 by reference to its UniProt ID. In some embodiments, the functional variant catalyzes the same reaction as the corresponding wild-type protein, e.g., at a rate no less or no more than 10%, 20%, 30%, 40%, or 50% lower than the wild-type protein.

TABLE 15
Exemplary enzymatic effectors for enzyme deficiency

| Effector | Deficiency | Entrez Gene ID¹ | UniProt ID² |
|---|---|---|---|
| 3-methylcrotonyl-CoA | 3-methylcrotonyl-CoA carboxylase | 56922, 64087 | Q96RQ3, |
| carboxylase | deficiency | | Q9HCC0 |
| Acetyl-CoA- | Mucopolysaccharidosis MPS III | 138050 | Q68CP4 |
| glucosaminide N- | (Sanfilippo's syndrome) Type III-C | | |
| acetyltransferase | | | |
| ADAMTS13 | Thrombotic Thrombocytopenia Purpura | 11093 | Q76LX8 |
| adenine | Adenine phosphoribosyltransferase | 353 | P07741 |
| phosphoribosyltransferase | deficiency | | |
| Adenosine deaminase | Adenosine deaminase deficiency | 100 | P00813 |
| ADP-ribose protein | Glutamyl ribose-5-phosphate storage | 26119, 54936 | Q5SW96, |
| hydrolase | disease | | Q9NX46 |
| alpha glucosidase | Glycogen storage disease type 2 | 2548 | P10253 |
| | (Pompe's disease) | | |
| Arginase | Familial hyperarginemia | 383, 384 | P05089, |
| | | | P78540 |
| Arylsulfatase A | Metachromatic leukodystrophy | 410 | P15289 |
| Cathepsin K | Pycnodysostosis | 1513 | P43235 |
| Ceramidase | Farber's disease (lipogranulomatosis) | 125981, 340485, 55331 | Q8TDN7, |
| | | | Q5QJU3, |
| | | | Q9NUN7 |
| Cystathionine B | Homocystinuria | 875 | P35520 |
| synthase | | | |
| Dolichol-P-mannose | Congenital disorders of N-glycosylation | 8813, 54344 | O60762, |
| synthase | CDG Ie | | Q9P2X0 |
| Dolicho-P- | Congenital disorders of N-glycosylation | 84920 | Q5BKT4 |
| Glc:Man9GlcNAc2-PP- | CDG Ic | | |
| dolichol | | | |
| glucosyltransferase | | | |
| Dolicho-P- | Congenital disorders of N-glycosylation | 10195 | Q92685 |
| Man:Man5GlcNAc2- | CDG Id | | |
| PP-dolichol | | | |
| mannosyltransferase | | | |
| Dolichyl-P-glucose:Glc- | Congenital disorders of N-glycosylation | 79053 | Q9BVK2 |
| 1-Man-9-GlcNAc-2-PP- | CDG Ih | | |
| dolichyl-α-3- | | | |
| glucosyltransferase | | | |
| Dolichyl-P- | Congenital disorders of N-glycosylation | 79087 | Q9BV10 |
| mannose:Man-7- | CDG Ig | | |
| GlcNAc-2-PP-dolichyl- | | | |
| α-6- | | | |
| mannosyltransferase | | | |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor Deficiency | 2159 | P00742 |
| Factor XI | Hemophilia C | 2160 | P03951 |
| Factor XIII | Fibrin Stabilizing factor deficiency | 2162, 2165 | P00488, |
| | | | P05160 |
| Galactosamine-6- | Mucopolysaccharidosis MPS IV | 2588 | P34059 |
| sulfate sulfatase | (Morquio's syndrome) Type IV-A | | |
| Galactosylceramide β- | Krabbe's disease | 2581 | P54803 |
| galactosidase | | | |
| Ganglioside β- | GM1 gangliosidosis, generalized | 2720 | P16278 |
| galactosidase | | | |
| Ganglioside β- | GM2 gangliosidosis | 2720 | P16278 |
| galactosidase | | | |
| Ganglioside β- | Sphingolipidosis Type I | 2720 | P16278 |
| galactosidase | | | |
| Ganglioside β- | Sphingolipidosis Type II (juvenile type) | 2720 | P16278 |
| galactosidase | | | |
| Ganglioside β- | Sphingolipidosis Type III (adult type) | 2720 | P16278 |
| galactosidase | | | |
| Glucosidase I | Congenital disorders of N-glycosylation | 2548 | P10253 |
| | CDG IIb | | |
| Glucosylceramide β- | Gaucher's disease | 2629 | P04062 |
| glucosidase | | | |
| Heparan-S-sulfate | Mucopolysaccharidosis MPS III | 6448 | P51688 |
| sulfamidase | (Sanfilippo's syndrome) Type III-A | | |
| homogentisate oxidase | Alkaptonuria | 3081 | Q93099 |
| Hyaluronidase | Mucopolysaccharidosis MPS IX | 3373, 8692, | Q12794, |
| | (hyaluronidase deficiency) | 8372, 23553 | Q12891, |
| | | | O43820, |
| | | | Q2M3T9 |
| Iduronate sulfate | Mucopolysaccharidosis MPS II | 3423 | P22304 |
| sulfatase | (Hunter's syndrome) | | |
| Lecithin-cholesterol | Complete LCAT deficiency, Fish-eye | 3931 | 606967 |
| acyltransferase (LCAT) | disease, atherosclerosis, | | |
| | hypercholesterolemia | | |
| Lysine oxidase | Glutaric acidemia type I | 4015 | P28300 |
| Lysosomal acid lipase | Cholesteryl ester storage disease | 3988 | P38571 |
| | (CESD) | | |
| Lysosomal acid lipase | Lysosomal acid lipase deficiency | 3988 | P38571 |
| lysosomal acid lipase | Wolman's disease | 3988 | P38571 |
| Lysosomal pepstatin- | Ceroid lipofuscinosis Late infantile form | 1200 | O14773 |
| insensitive peptidase | (CLN2, Jansky-Bielschowsky disease) | | |
| Mannose (Man) | Congenital disorders of N-glycosylation | 4351 | P34949 |
| phosphate (P) | CDG Ib | | |
| isomerase | | | |
| Mannosyl-α-1,6- | Congenital disorders of N-glycosylation | 4247 | Q10469 |
| glycoprotein-β-1,2-N- | CDG IIa | | |
| acetylglucosminyltransferase | | | |
| Metalloproteinase-2 | Winchester syndrome | 4313 | P08253 |
| methylmalonyl-CoA | Methylmalonic acidemia (vitamin b12 | 4594 | P22033 |
| mutase | non-responsive) | | |
| N-Acetyl | Mucopolysaccharidosis MPS VI | 411 | P15848 |
| galactosamine α-4- | (Maroteaux-Lamy syndrome) | | |
| sulfate sulfatase | | | |
| (arylsulfatase B) | | | |
| N-acetyl-D- | Mucopolysaccharidosis MPS III | 4669 | P54802 |
| glucosaminidase | (Sanfilippo's syndrome) Type III-B | | |
| N-Acetyl- | Schindler's disease Type I (infantile | 4668 | P17050 |
| galactosaminidase | severe form) | | |
| N-Acetyl- | Schindler's disease Type II (Kanzaki | 4668 | P17050 |
| galactosaminidase | disease, adult-onset form) | | |
| N-Acetyl- | Schindler's disease Type III | 4668 | P17050 |
| galactosaminidase | (intermediate form) | | |
| N-acetyl- | Mucopolysaccharidosis MPS III | 2799 | P15586 |
| glucosaminine-6- | (Sanfilippo's syndrome) Type III-D | | |
| sulfate sulfatase | | | |
| N-acetylglucosaminyl- | Mucolipidosis ML III (pseudo-Hurler's | 79158 | Q3T906 |
| 1-phosphotransferase | polydystrophy) | | |
| N-Acetylglucosaminyl- | Mucolipidosis ML II (I-cell disease) | 79158 | Q3T906 |
| 1-phosphotransferase | | | |
| catalytic subunit | | | |
| N-acetylglucosaminyl- | Mucolipidosis ML III (pseudo-Hurler's | 84572 | Q9UJJ9 |
| 1-phosphotransferase, | polydystrophy) Type III-C | | |
| substrate-recognition | | | |
| subunit | | | |
| N- | Aspartylglucosaminuria | 175 | P20933 |
| Aspartylglucosaminidase | | | |
| Neuraminidase 1 | Sialidosis | 4758 | Q99519 |
| (sialidase) | | | |
| Palmitoyl-protein | Ceroid lipofuscinosis Adult form (CLN4, | 5538 | P50897 |
| thioesterase-1 | Kufs' disease) | | |
| Palmitoyl-protein | Ceroid lipofuscinosis Infantile form | 5538 | P50897 |
| thioesterase-1 | (CLN1, Santavuori-Haltia disease) | | |
| Phenylalanine | Phenylketonuria | 5053 | P00439 |
| hydroxylase | | | |
| Phosphomannomutase- | Congenital disorders of N-glycosylation | 5373 | O15305 |
| 2 | CDG Ia (solely neurologic and | | |
| | neurologic-multivisceral forms) | | |
| Porphobilinogen | Acute Intermittent Porphyria | 3145 | P08397 |
| deaminase | | | |
| Purine nucleoside | Purine nucleoside phosphorylase | 4860 | P00491 |
| phosphorylase | deficiency | | |
| pyrimidine 5′ | Hemolytic anemia and/or pyrimidine 5′ | 51251 | Q9H0P0 |
| nucleotidase | nucleotidase deficiency | | |
| Sphingomyelinase | Niemann-Pick disease type A | 6609 | P17405 |
| Sphingomyelinase | Niemann-Pick disease type B | 6609 | P17405 |
| Sterol 27-hydroxylase | Cerebrotendinous xanthomatosis | 1593 | Q02318 |
| | (cholestanol lipidosis) | | |
| Thymidine | Mitochondrial neurogastrointestinal | 1890 | P19971 |
| phosphorylase | encephalomyopathy (MNGIE) | | |
| Trihexosylceramide α- | Fabry's disease | 2717 | P06280 |
| galactosidase | | | |
| tyrosinase, e.g., OCA1 | albinism, e.g., ocular albinism | 7299 | P14679 |
| UDP-GlcNAc:dolichyl-P | Congenital disorders of N-glycosylation | 1798 | Q9H3H5 |
| NAcGlc | CDG Ij | | |
| phosphotransferase | | | |
| UDP-N- | Sialuria French type | 10020 | Q9Y223 |
| acetylglucosamine-2- | | | |
| epimerase/N- | | | |
| acetylmannosamine | | | |
| kinase, sialin | | | |
| Uricase | Lesch-Nyhan syndrome, gout | 391051 | No protein |
| uridine diphosphate | Crigler-Najjar syndrome | 54658 | P22309 |
| glucuronyl-transferase | | | |
| (e.g., UGT1A1) | | | |
| α-1,2- | Congenital disorders of N-glycosylation | 79796 | Q9H6U8 |
| Mannosyltransferase | CDG II (608776) | | |
| α-1,2- | Congenital disorders of N-glycosylation, | 79796 | Q9H6U8 |
| Mannosyltransferase | type I (pre-Golgi glycosylation defects) | | |
| α-1,3- | Congenital disorders of N-glycosylation | 440138 | Q2TAA5 |
| Mannosyltransferase | CDG Ii | | |
| α-D-Mannosidase | α-Mannosidosis, type I (severe) or II | 10195 | Q92685 |
| | (mild) | | |
| α-L-Fucosidase | Fucosidosis | 4123 | Q9NTJ4 |
| α-l-Iduronidase | Mucopolysaccharidosis MPS I H/S | 2517 | P04066 |
| | (Hurler-Scheie syndrome) | | |
| α-l-Iduronidase | Mucopolysaccharidosis MPS I-H | 3425 | P35475 |
| | (Hurler's syndrome) | | |
| α-l-Iduronidase | Mucopolysaccharidosis MPS I-S | 3425 | P35475 |
| | (Scheie's syndrome) | | |
| β-1,4- | Congenital disorders of N-glycosylation | 3425 | P35475 |
| Galactosyltransferase | CDG IId | | |
| β-1,4- | Congenital disorders of N-glycosylation | 2683 | P15291 |
| Mannosyltransferase | CDG Ik | | |
| β-D-Mannosidase | β-Mannosidosis | 56052 | Q9BT22 |
| β-Galactosidase | Mucopolysaccharidosis MPS IV | 4126 | O00462 |
| | (Morquio's syndrome) Type IV-B | | |
| β-Glucuronidase | Mucopolysaccharidosis MPS VII (Sly's | 2720 | P16278 |
| | syndrome) | | |
| β-Hexosaminidase A | Tay-Sachs disease | 2990 | P08236 |
| β-Hexosaminidase B | Sandhoff's disease | 3073 | P06865 |

¹Sequence available on the NCBI database on the world wide web internet site ncbi.nlm.nih.gov/gene.
²Sequence available on the Uniprot database on the world wide web internet site uniprot.org/uniprot.

### Other Non-Enzymatic Effectors

In some embodiments, a therapeutic polypeptide described herein comprises a polypeptide of Table 16, or a functional variant thereof, e.g., a protein having at least 80%, 85%, 90%, 95%, 967%, 98%, 99% identity to a protein sequence disclosed in Table 16 by reference to its UniProt ID.

TABLE 16
Exemplary non-enzymatic effectors and corresponding indications

| | | Entrez | |
|---|---|---|---|
| | | Gene | UniProt |
| Effector | Indication | ID¹ | ID² |
| Survival motor | spinal muscular atrophy | 6606 | Q16637 |
| neuron protein | | | |
| (SMN) | | | |
| Dystrophin | muscular dystrophy (e.g., Duchenne muscular | 1756 | P11532 |
| | dystrophy or Becker muscular dystrophy) | | |
| Complement | Complement Factor I deficiency | 3426 | P05156 |
| protein, e.g., | | | |
| Complement | | | |
| factor C1 | | | |
| Complement | Atypical hemolytic uremic syndrome | 3075 | P08603 |
| factor H | | | |
| Cystinosin | Cystinosis | 1497 | O60931 |
| (lysosomal | | | |
| cystine | | | |
| transporter) | | | |
| Epididymal | Niemann-Pick disease Type C2 | 10577 | P61916 |
| secretory protein | | | |
| 1 (HE1; NPC2 | | | |
| protein) | | | |
| GDP-fucose | Congenital disorders of N-glycosylation CDG IIc | 55343 | Q96A29 |
| transporter-1 | (Rambam-Hasharon syndrome) | | |
| GM2 activator | GM2 activator protein deficiency (Tay-Sachs | 2760 | Q17900 |
| protein | disease AB variant, GM2A) | | |
| Lysosomal | Ceroid lipofuscinosis Juvenile form (CLN3, Batten | 1207 | Q13286 |
| transmembrane | disease, Vogt-Spielmeyer disease) | | |
| CLN3 protein | | | |
| Lysosomal | Ceroid lipofuscinosis Variant late infantile form, | 1203 | O75503 |
| transmembrane | Finnish type (CLN5) | | |
| CLN5 protein | | | |
| Na phosphate | Infantile sialic acid storage disorder | 26503 | Q9NRA2 |
| cotransporter, | | | |
| sialin | | | |
| Na phosphate | Sialuria Finnish type (Salla disease) | 26503 | Q9NRA2 |
| cotransporter, | | | |
| sialin | | | |
| NPC1 protein | Niemann-Pick disease Type C1/Type D | 4864 | O15118 |
| Oligomeric Golgi | Congenital disorders of N-glycosylation CDG IIe | 91949 | P83436 |
| complex-7 | | | |
| Prosaposin | Prosaposin deficiency | 5660 | P07602 |
| Protective | Galactosialidosis (Goldberg's syndrome, | 5476 | P10619 |
| protein/cathepsin | combined neuraminidase and β-galactosidase | | |
| A (PPCA) | deficiency) | | |
| Protein involved | Congenital disorders of N-glycosylation CDG If | 9526 | O75352 |
| in mannose-P- | | | |
| dolichol | | | |
| utilization | | | |
| Saposin B | Saposin B deficiency (sulfatide activator | 5660 | P07602 |
| | deficiency) | | |
| Saposin C | Saposin C deficiency (Gaucher's activator | 5660 | P07602 |
| | deficiency) | | |
| Sulfatase- | Mucosulfatidosis (multiple sulfatase deficiency) | 285362 | Q8NBK3 |
| modifying | | | |
| factor-1 | | | |
| Transmembrane | Ceroid lipofuscinosis Variant late infantile form | 54982 | Q9NWW5 |
| CLN6 protein | (CLN6) | | |
| Transmembrane | Ceroid lipofuscinosis Progressive epilepsy with | 2055 | Q9UBY8 |
| CLN8 protein | intellectual disability | | |
| vWF | von Willebrand disease | 7450 | P04275 |
| Factor I | Afibrinogenomia | 2243, | P02671, |
| (fibrinogen) | | 2244, 2266 | P02675, P02679 |
| erythropoietin | Anemia | 2056 | P01588 |
| (hEPO) | | | |

¹Sequence available on the NCBI database on the world wide web internet site ncbi.nlm.nih.gov/gene.
²Sequence available on the Uniprot database on the world wide web internet site uniprot.org/uniprot.

### Regeneration, Repair and Fibrosis Factors

Therapeutic polypeptides described herein also include growth factors, e.g., as disclosed in Table 17, or functional variants thereof, e.g., a protein having at least 80%, 85%, 90%, 95%, 967%, 98%, 99% identity to a protein sequence disclosed in Table 17 by reference to its NCBI protein accession number. Also included are antibodies or fragments thereof against such growth factors, or miRNAs that promote regeneration and repair.

TABLE 17
Exemplary Regeneration, Repair, and Fibrosis Factors

| | NCBI Gene | NCBI Protein |
|---|---|---|
| Target | accession # ¹ | accession # ² |
| VEGF-A | NG_008732 | NP_001165094 |
| NRG-1 | NG_012005 | NP_001153471 |
| FGF2 | NG_029067 | NP_001348594 |
| FGF1 | Gene ID: 2246 | NP_001341882 |
| miR199-3p | MIMAT0000232 | n/a |
| miR590-3p | MIMAT0004801 | n/a |
| miR17-92 | MI0000071 | On the world wide web internet site |
| | | “ncbi.nlm.nih.gov/pmc/articles/ |
| | | PMC2732113/figure/F1/” |
| miR222 | MI0000299 | n/a |
| miR302-367 | MIR302A And | On the world wide web internet site |
| | MIR367 | “ncbi.nlm.nih.gov/pmc/articles/ |
| | | PMC4400607/” |

¹ Sequence available on the world wide web internet site ncbi.nlm.nih.gov/gene.
² Sequence available on the world wide web internet site ncbi.nlm.nih.gov/protein.

### Transformation Factors

Therapeutic polypeptides described herein also include transformation factors, e.g., protein factors that transform fibroblasts into differentiated cell e.g., factors disclosed in Table 18 or functional variants thereof, e.g., a protein having at least 80%, 85%, 90%, 95%, 967%, 98%, 99% identity to a protein sequence disclosed in Table 18 by reference to its UniProt ID.

TABLE 18
Polypeptides indicated for organ repair by transforming fibroblasts

| | NCBI Gene | NCBI Protein |
|---|---|---|
| Target | accession # ¹ | accession # ² |
| MESP1 | Gene ID: 55897 | EAX02066 |
| ETS2 | GeneID: 2114 | NP_005230 |
| HAND2 | GeneID: 9464 | NP_068808 |
| MYOCARDIN | GeneID: 93649 | NP_001139784 |
| ESRRA | Gene ID: 2101 | AAH92470 |
| miR1 | MI0000651 | n/a |
| miR133 | MI000450 | n/a |
| TGFb | GeneID: 7040 | NP_000651.3 |
| WNT | Gene ID: 7471 | NP_005421 |
| JAK | Gene ID: 3716 | NP_001308784 |
| NOTCH | GenelD: 4851 | XP_011517019 |

¹ Sequence available on the world wide web internet site ncbi.nlm.nih.gov/gene.
² Sequence available on the world wide web internet site ncbi.nlm.nih.gov/protein.

Proteins that Stimulate Cellular Regeneration

Therapeutic polypeptides described herein also include proteins that stimulate cellular regeneration e.g., proteins disclosed in Table 19 or functional variants thereof, e.g., a protein having at least 80%, 85%, 90%, 95%, 967%, 98%, 99% identity to a protein sequence disclosed in Table 19 by reference to its UniProt ID.

TABLE 19
Exemplary proteins that stimulate cellular regeneration

| Target | Gene accession # ¹ | Protein accession # ² |
|---|---|---|
| MST1 | NG_016454 | NP_066278 |
| STK30 | Gene ID: 26448 | NP_036103 |
| MST2 | Gene ID: 6788 | NP_006272 |
| SAV1 | Gene ID: 60485 | NP_068590 |
| LATS1 | Gene ID: 9113 | NP_004681 |
| LATS2 | Gene ID: 26524 | NP_055387 |
| YAP1 | NG_029530 | NP_001123617 |
| CDKN2b | NG_023297 | NP_004927 |
| CDKN2a | NG_007485 | NP_478102 |

¹ Sequence available on the world wide web internet site ncbi.nlm.nih.gov/gene.
² Sequence available on the world wide web internet site ncbi.nlm.nih.gov/protein.

### Cystic Fibrosis Transmembrane Conductance Regulator (CFTR)

In some embodiments, an effector described herein comprises CFTR gene of Table 20, or a functional variant or fragment thereof, e.g., a protein having at least 80%, 85%, 90%, 95%, 96%, 97%, 98%, 99% identity to a protein sequence disclosed in Table 20.

TABLE 20
Exemplary CFTR genes

| Accession | | |
|---|---|---|
| Number | DNA sequence | Amino Acid sequence |
| NM_000492.4 | ATGCAGAGGTCGCCTCTGGAAAAGGCCAGCGTTGTCTCCAAACTTTTTTTCAGC | MQRSPLEKASVVSKLFFSWTRPILRKGYRQR |
| | TGGACCAGACCAATTTTGAGGAAAGGATACAGACAGCGCCTGGAATTGTCAGA | LELSDIYQIPSVDSADNLSEKLEREWDRELAS |
| | CATATACCAAATCCCTTCTGTTGATTCTGCTGACAATCTATCTGAAAAATTGGAA | KKNPKLINALRRCFFWRFMFYGIFLYLGEVTK |
| | AGAGAATGGGATAGAGAGCTGGCTTCAAAGAAAAATCCTAAACTCATTAATGCC | AVQPLLLGRIIASYDPDNKEERSIAIYLGIGLCL |
| | CTTCGGCGATGTTTTTTCTGGAGATTTATGTTCTATGGAATCTTTTTATATTTAGG | LFIVRTLLLHPAIFGLHHIGMQMRIAMFSLIYKK |
| | GGAAGTCACCAAAGCAGTACAGCCTCTCTTACTGGGAAGAATCATAGCTTCCTA | TLKLSSRVLDKISIGQLVSLLSNNLNKFDEGLA |
| | TGACCCGGATAACAAGGAGGAACGCTCTATCGCGATTTATCTAGGCATAGGCTT | LAHFVWIAPLQVALLMGLIWELLQASAFCGLG |
| | ATGCCTTCTCTTTATTGTGAGGACACTGCTCCTACACCCAGCCATTTTTGGCCTT | FLIVLALFQAGLGRMMMKYRDQRAGKISERL |
| | CATCACATTGGAATGCAGATGAGAATAGCTATGTTTAGTTTGATTTATAAGAAGA | VITSEMIENIQSVKAYCWEEAMEKMIENLRQT |
| | CTTTAAAGCTGTCAAGCCGTGTTCTAGATAAAATAAGTATTGGACAACTTGTTAG | ELKLTRKAAYVRYFNSSAFFFSGFFVVFLSVL |
| | TCTCCTTTCCAACAACCTGAACAAATTTGATGAAGGACTTGCATTGGCACATTTC | PYALIKGIILRKIFTTISFCIVLRMAVTRQFPWA |
| | GTGTGGATCGCTCCTTTGCAAGTGGCACTCCTCATGGGGCTAATCTGGGAGTT | VQTWYDSLGAINKIQDFLQKQEYKTLEYNLTT |
| | GTTACAGGCGTCTGCCTTCTGTGGACTTGGTTTCCTGATAGTCCTTGCCCTTTT | TEVVMENVTAFWEEGFGELFEKAKQNNNNR |
| | TCAGGCTGGGCTAGGGAGAATGATGATGAAGTACAGAGATCAGAGAGCTGGGA | KTSNGDDSLFFSNFSLLGTPVLKDINFKIERG |
| | AGATCAGTGAAAGACTTGTGATTACCTCAGAAATGATTGAAAATATCCAATCTGT | QLLAVAGSTGAGKTSLLMVIMGELEPSEGKIK |
| | TAAGGCATACTGCTGGGAAGAAGCAATGGAAAAAATGATTGAAAACTTAAGACA | HSGRISFCSQFSWIMPGTIKENIIFGVSYDEYR |
| | AACAGAACTGAAACTGACTCGGAAGGCAGCCTATGTGAGATACTTCAATAGCTC | YRSVIKACQLEEDISKFAEKDNIVLGEGGITLS |
| | AGCCTTCTTCTTCTCAGGGTTCTTTGTGGTGTTTTTATCTGTGCTTCCCTATGCA | GGQRARISLARAVYKDADLYLLDSPFGYLDVL |
| | CTAATCAAAGGAATCATCCTCCGGAAAATATTCACCACCATCTCATTCTGCATTG | TEKEIFESCVCKLMANKTRILVTSKMEHLKKA |
| | TTCTGCGCATGGCGGTCACTCGGCAATTTCCCTGGGCTGTACAAACATGGTAT | DKILILHEGSSYFYGTFSELQNLQPDFSSKLM |
| | GACTCTCTTGGAGCAATAAACAAAATACAGGATTTCTTACAAAAGCAAGAATATA | GCDSFDQFSAERRNSILTETLHRFSLEGDAP |
| | AGACATTGGAATATAACTTAACGACTACAGAAGTAGTGATGGAGAATGTAACAG | VSWTETKKQSFKQTGEFGEKRKNSILNPINSI |
| | CCTTCTGGGAGGAGGGATTTGGGGAATTATTTGAGAAAGCAAAACAAAACAATA | RKFSIVQKTPLQMNGIEEDSDEPLERRLSLVP |
| | ACAATAGAAAAACTTCTAATGGTGATGACAGCCTCTTCTTCAGTAATTTCTCACT | DSEQGEAILPRISVISTGPTLQARRRQSVLNL |
| | TCTTGGTACTCCTGTCCTGAAAGATATTAATTTCAAGATAGAAAGAGGACAGTTG | MTHSVNQGQNIHRKTTASTRKVSLAPQANLT |
| | TTGGCGGTTGCTGGATCCACTGGAGCAGGCAAGACTTCACTTCTAATGGTGATT | ELDIYSRRLSQETGLEISEEINEEDLKECFFDD |
| | ATGGGAGAACTGGAGCCTTCAGAGGGTAAAATTAAGCACAGTGGAAGAATTTC | MESIPAVTTWNTYLRYITVHKSLIFVLIWCLVIF |
| | ATTCTGTTCTCAGTTTTCCTGGATTATGCCTGGCACCATTAAAGAAAATATCATC | LAEVAASLVVLWLLGNTPLQDKGNSTHSRNN |
| | TTTGGTGTTTCCTATGATGAATATAGATACAGAAGCGTCATCAAAGCATGCCAA | SYAVIITSTSSYYVFYIYVGVADTLLAMGFFRG |
| | CTAGAAGAGGACATCTCCAAGTTTGCAGAGAAAGACAATATAGTTCTTGGAGAA | LPLVHTLITVSKILHHKMLHSVLQAPMSTLNTL |
| | GGTGGAATCACACTGAGTGGAGGTCAACGAGCAAGAATTTCTTTAGCAAGAGC | KAGGILNRFSKDIAILDDLLPLTIFDFIQLLLIVIG |
| | AGTATACAAAGATGCTGATTTGTATTTATTAGACTCTCCTTTTGGATACCTAGAT | AIAVVAVLQPYIFVATVPVIVAFIMLRAYFLQTS |
| | GTTTTAACAGAAAAAGAAATATTTGAAAGCTGTGTCTGTAAACTGATGGCTAACA | QQLKQLESEGRSPIFTHLVTSLKGLWTLRAF |
| | AAACTAGGATTTTGGTCACTTCTAAAATGGAACATTTAAAGAAAGCTGACAAAAT | GRQPYFETLFHKALNLHTANWFLYLSTLRWF |
| | ATTAATTTTGCATGAAGGTAGCAGCTATTTTTATGGGACATTTTCAGAACTCCAA | QMRIEMIFVIFFIAVTFISILTTGEGEGRVGIILT |
| | AATCTACAGCCAGACTTTAGCTCAAAACTCATGGGATGTGATTCTTTCGACCAAT | LAMNIMSTLQWAVNSSIDVDSLMRSVSRVFK |
| | TTAGTGCAGAAAGAAGAAATTCAATCCTAACTGAGACCTTACACCGTTTCTCATT | FIDMPTEGKPTKSTKPYKNGQLSKVMIIENSH |
| | AGAAGGAGATGCTCCTGTCTCCTGGACAGAAACAAAAAAACAATCTTTTAAACA | VKKDDIWPSGGQMTVKDLTAKYTEGGNAILE |
| | GACTGGAGAGTTTGGGGAAAAAAGGAAGAATTCTATTCTCAATCCAATCAACTC | NISFSISPGQRVGLLGRTGSGKSTLLSAFLRL |
| | TATACGAAAATTTTCCATTGTGCAAAAGACTCCCTTACAAATGAATGGCATCGAA | LNTEGEIQIDGVSWDSITLQQWRKAFGVIPQK |
| | GAGGATTCTGATGAGCCTTTAGAGAGAAGGCTGTCCTTAGTACCAGATTCTGAG | VFIFSGTFRKNLDPYEQWSDQEIWKVADEVG |
| | CAGGGAGAGGCGATACTGCCTCGCATCAGCGTGATCAGCACTGGCCCCACGC | LRSVIEQFPGKLDFVLVDGGCVLSHGHKQLM |
| | TTCAGGCACGAAGGAGGCAGTCTGTCCTGAACCTGATGACACACTCAGTTAAC | CLARSVLSKAKILLLDEPSAHLDPVTYQIIRRT |
| | CAAGGTCAGAACATTCACCGAAAGACAACAGCATCCACACGAAAAGTGTCACT | LKQAFADCTVILCEHRIEAMLECQQFLVIEEN |
| | GGCCCCTCAGGCAAACTTGACTGAACTGGATATATATTCAAGAAGGTTATCTCA | KVRQYDSIQKLLNERSLFRQAISPSDRVKLFP |
| | AGAAACTGGCTTGGAAATAAGTGAAGAAATTAACGAAGAAGACTTAAAGGAGTG | HRNSSKCKSKPQIAALKEETEEEVQDTRL |
| | CTTTTTTGATGATATGGAGAGCATACCAGCAGTGACTACATGGAACACATACCT | (SEQ ID NO: 258) |
| | TCGATATATTACTGTCCACAAGAGCTTAATTTTTGTGCTAATTTGGTGCTTAGTA | |
| | ATTTTTCTGGCAGAGGTGGCTGCTTCTTTGGTTGTGCTGTGGCTCCTTGGAAAC | |
| | ACTCCTCTTCAAGACAAAGGGAATAGTACTCATAGTAGAAATAACAGCTATGCA | |
| | GTGATTATCACCAGCACCAGTTCGTATTATGTGTTTTACATTTACGTGGGAGTAG | |
| | CCGACACTTTGCTTGCTATGGGATTCTTCAGAGGTCTACCACTGGTGCATACTC | |
| | TAATCACAGTGTCGAAAATTTTACACCACAAAATGTTACATTCTGTTCTTCAAGC | |
| | ACCTATGTCAACCCTCAACACGTTGAAAGCAGGTGGGATTCTTAATAGATTCTC | |
| | CAAAGATATAGCAATTTTGGATGACCTTCTGCCTCTTACCATATTTGACTTCATC | |
| | CAGTTGTTATTAATTGTGATTGGAGCTATAGCAGTTGTCGCAGTTTTACAACCCT | |
| | ACATCTTTGTTGCAACAGTGCCAGTGATAGTGGCTTTTATTATGTTGAGAGCATA | |
| | TTTCCTCCAAACCTCACAGCAACTCAAACAACTGGAATCTGAAGGCAGGAGTCC | |
| | AATTTTCACTCATCTTGTTACAAGCTTAAAAGGACTATGGACACTTCGTGCCTTC | |
| | GGACGGCAGCCTTACTTTGAAACTCTGTTCCACAAAGCTCTGAATTTACATACT | |
| | GCCAACTGGTTCTTGTACCTGTCAACACTGCGCTGGTTCCAAATGAGAATAGAA | |
| | ATGATTTTTGTCATCTTCTTCATTGCTGTTACCTTCATTTCCATTTTAACAACAGG | |
| | AGAAGGAGAAGGAAGAGTTGGTATTATCCTGACTTTAGCCATGAATATCATGAG | |
| | TACATTGCAGTGGGCTGTAAACTCCAGCATAGATGTGGATAGCTTGATGCGATC | |
| | TGTGAGCCGAGTCTTTAAGTTCATTGACATGCCAACAGAAGGTAAACCTACCAA | |
| | GTCAACCAAACCATACAAGAATGGCCAACTCTCGAAAGTTATGATTATTGAGAA | |
| | TTCACACGTGAAGAAAGATGACATCTGGCCCTCAGGGGGCCAAATGACTGTCA | |
| | AAGATCTCACAGCAAAATACACAGAAGGTGGAAATGCCATATTAGAGAACATTT | |
| | CCTTCTCAATAAGTCCTGGCCAGAGGGTGGGCCTCTTGGGAAGAACTGGATCA | |
| | GGGAAGAGTACTTTGTTATCAGCTTTTTTGAGACTACTGAACACTGAAGGAGAA | |
| | ATCCAGATCGATGGTGTGTCTTGGGATTCAATAACTTTGCAACAGTGGAGGAAA | |
| | GCCTTTGGAGTGATACCACAGAAAGTATTTATTTTTTCTGGAACATTTAGAAAAA | |
| | ACTTGGATCCCTATGAACAGTGGAGTGATCAAGAAATATGGAAAGTTGCAGATG | |
| | AGGTTGGGCTCAGATCTGTGATAGAACAGTTTCCTGGGAAGCTTGACTTTGTCC | |
| | TTGTGGATGGGGGCTGTGTCCTAAGCCATGGCCACAAGCAGTTGATGTGCTTG | |
| | GCTAGATCTGTTCTCAGTAAGGCGAAGATCTTGCTGCTTGATGAACCCAGTGCT | |
| | CATTTGGATCCAGTAACATACCAAATAATTAGAAGAACTCTAAAACAAGCATTTG | |
| | CTGATTGCACAGTAATTCTCTGTGAACACAGGATAGAAGCAATGCTGGAATGCC | |
| | AACAATTTTTGGTCATAGAAGAGAACAAAGTGCGGCAGTACGATTCCATCCAGA | |
| | AACTGCTGAACGAGAGGAGCCTCTTCCGGCAAGCCATCAGCCCCTCCGACAG | |
| | GGTGAAGCTCTTTCCCCACCGGAACTCAAGCAAGTGCAAGTCTAAGCCCCAGA | |
| | TTGCTGCTCTGAAAGAGGAGACAGAAGAAGAGGTGCAAGATACAAGGCTTTAG | |
| | (SEQ ID NO: 257) | |
| N/A | ATGCAGCGGTCCCCGCTCGAAAAGGCCAGTGTCGTGTCCAAACTCTTCTTCTC | MQRSPLEKASVVSKLFFSWTRPILRKGYRQR |
| | ATGGACTCGGCCTATCCTTAGAAAGGGGTATCGGCAGAGGCTTGAGTTGTCTG | LELSDIYQIPSVDSADNLSEKLEREWDRELAS |
| | ACATCTACCAGATCCCCTCGGTAGATTCGGCGGATAACCTCTCGGAGAAGCTC | KKNPKLINALRRCFFWRFMFYGIFLYLGEVTK |
| | GAACGGGAATGGGACCGCGAACTCGCGTCTAAGAAAAACCCGAAGCTCATCAA | AVQPLLLGRIIASYDPDNKEERSIAIYLGIGLCL |
| | CGCACTGAGAAGGTGCTTCTTCTGGCGGTTCATGTTCTACGGTATCTTCTTGTA | LFIVRTLLLHPAIFGLHHIGMQMRIAMFSLIYKK |
| | TCTCGGGGAGGTCACAAAAGCAGTCCAACCCCTGTTGTTGGGTCGCATTATCG | TLKLSSRVLDKISIGQLVSLLSNNLNKFDEGLA |
| | CCTCGTACGACCCCGATAACAAAGAAGAACGGAGCATCGCGATCTACCTCGGG | LAHFVWIAPLQVALLMGLIWELLQASAFCGLG |
| | ATCGGACTGTGTTTGCTTTTCATCGTCAGAACACTTTTGTTGCATCCAGCAATCT | FLIVLALFQAGLGRMMMKYRDQRAGKISERL |
| | TCGGCCTCCATCACATCGGTATGCAGATGCGAATCGCTATGTTTAGCTTGATCT | VITSEMIENIQSVKAYCWEEAMEKMIENLRQT |
| | ACAAAAAGACACTGAAACTCTCGTCGCGGGTGTTGGATAAGATTTCCATCGGTC | ELKLTRKAAYVRYFNSSAFFFSGFFVVFLSVL |
| | AGTTGGTGTCCCTGCTTAGTAATAACCTCAACAAATTCGATGAGGGACTGGCGC | PYALIKGIILRKIFTTISFCIVLRMAVTRQFPWA |
| | TGGCACATTTCGTGTGGATTGCCCCGTTGCAAGTCGCCCTTTTGATGGGCCTTA | VQTWYDSLGAINKIQDFLQKQEYKTLEYNLTT |
| | TTTGGGAGCTGTTGCAGGCATCTGCCTTTTGTGGCCTGGGATTTCTGATTGTGT | TEVVMENVTAFWEEGFGELFEKAKQNNNNR |
| | TGGCATTGTTTCAGGCTGGGCTTGGGCGGATGATGATGAAGTATCGCGACCAG | KTSNGDDSLFFSNFSLLGTPVLKDINFKIERG |
| | AGAGCGGGTAAAATCTCGGAAAGACTCGTCATCACTTCGGAAATGATCGAAAAC | QLLAVAGSTGAGKTSLLMVIMGELEPSEGKIK |
| | ATCCAGTCGGTCAAAGCCTATTGCTGGGAAGAAGCTATGGAGAAGATGATTGA | HSGRISFCSQFSWIMPGTIKENIIFGVSYDEYR |
| | AAACCTCCGCCAAACTGAGCTGAAACTGACCCGCAAGGCGGCGTATGTCCGGT | YRSVIKACQLEEDISKFAEKDNIVLGEGGITLS |
| | ATTTCAATTCGTCAGCGTTCTTCTTTTCCGGGTTCTTCGTTGTCTTTCTCTCGGT | GGQRARISLARAVYKDADLYLLDSPFGYLDVL |
| | TTTGCCTTATGCCTTGATTAAGGGGATTATCCTCCGCAAGATTTTCACCACGATT | TEKEIFESCVCKLMANKTRILVTSKMEHLKKA |
| | TCGTTCTGCATTGTATTGCGCATGGCAGTGACACGGCAATTTCCGTGGGCCGT | DKILILHEGSSYFYGTFSELQNLQPDFSSKLM |
| | GCAGACATGGTATGACTCGCTTGGAGCGATCAACAAAATCCAAGACTTCTTGCA | GCDSFDQFSAERRNSILTETLHRFSLEGDAP |
| | AAAGCAAGAGTACAAGACCCTGGAGTACAATCTTACTACTACGGAGGTAGTAAT | VSWTETKKQSFKQTGEFGEKRKNSILNPINSI |
| | GGAGAATGTGACGGCTTTTTGGGAAGAGGGTTTTGGAGAACTGTTTGAGAAAG | RKFSIVQKTPLQMNGIEEDSDEPLERRLSLVP |
| | CAAAGCAGAATAACAACAACCGCAAGACCTCAAATGGGGACGATTCCCTGTTTT | DSEQGEAILPRISVISTGPTLQARRRQSVLNL |
| | TCTCGAACTTCTCCCTGCTCGGAACACCCGTGTTGAAGGACATCAATTTCAAGA | MTHSVNQGQNIHRKTTASTRKVSLAPQANLT |
| | TTGAGAGGGGACAGCTTCTCGCGGTAGCGGGAAGCACTGGTGCGGGAAAAAC | ELDIYSRRLSQETGLEISEEINEEDLKECFFDD |
| | TAGCCTCTTGATGGTGATTATGGGGGAGCTTGAGCCCAGCGAGGGGAAGATTA | MESIPAVTTWNTYLRYITVHKSLIFVLIWCLVIF |
| | AACACTCCGGGCGTATCTCATTCTGTAGCCAGTTTTCATGGATCATGCCCGGAA | LAEVAASLVVLWLLGNTPLQDKGNSTHSRNN |
| | CCATTAAAGAGAACATCATTTTCGGAGTATCCTATGATGAGTACCGATACAGAT | SYAVIITSTSSYYVFYIYVGVADTLLAMGFFRG |
| | CGGTCATTAAGGCGTGCCAGTTGGAAGAGGACATTTCTAAGTTCGCCGAGAAG | LPLVHTLITVSKILHHKMLHSVLQAPMSTLNTL |
| | GATAACATCGTCTTGGGAGAAGGGGGTATTACATTGTCGGGAGGGCAGCGAGC | KAGGILNRFSKDIAILDDLLPLTIFDFIQLLLIVIG |
| | GCGGATCAGCCTCGCGAGAGCGGTATACAAAGATGCAGATTTGTATCTGCTTG | AIAVVAVLQPYIFVATVPVIVAFIMLRAYFLQTS |
| | ATTCACCGTTTGGATACCTCGACGTATTGACAGAAAAAGAAATCTTCGAGTCGT | QQLKQLESEGRSPIFTHLVTSLKGLWTLRAF |
| | GCGTGTGTAAACTTATGGCTAATAAGACGAGAATCCTGGTGACATCAAAAATGG | GRQPYFETLFHKALNLHTANWFLYLSTLRWF |
| | AACACCTTAAGAAGGCGGACAAGATCCTGATCCTCCACGAAGGATCGTCCTAC | QMRIEMIFVIFFIAVTFISILTTGEGEGRVGIILT |
| | TTTTACGGCACTTTCTCAGAGTTGCAAAACTTGCAGCCGGACTTCTCAAGCAAA | LAMNIMSTLQWAVNSSIDVDSLMRSVSRVFK |
| | CTCATGGGGTGTGACTCATTCGACCAGTTCAGCGCGGAACGGCGGAACTCGAT | FIDMPTEGKPTKSTKPYKNGQLSKVMIIENSH |
| | CTTGACGGAAACGCTGCACCGATTCTCGCTTGAGGGTGATGCCCCGGTATCGT | VKKDDIWPSGGQMTVKDLTAKYTEGGNAILE |
| | GGACCGAGACAAAGAAGCAGTCGTTTAAGCAGACAGGAGAATTTGGTGAGAAA | NISFSISPGQRVGLLGRTGSGKSTLLSAFLRL |
| | AGAAAGAACAGTATCTTGAATCCTATTAACTCAATTCGCAAGTTCTCAATCGTCC | LNTEGEIQIDGVSWDSITLQQWRKAFGVIPQK |
| | AGAAAACTCCACTGCAGATGAATGGAATTGAAGAGGATTCGGACGAACCCCTG | VFIFSGTFRKNLDPYEQWSDQEIWKVADEVG |
| | GAGCGCAGGCTTAGCCTCGTGCCGGATTCAGAGCAAGGGGAGGCCATTCTTC | LRSVIEQFPGKLDFVLVDGGCVLSHGHKQLM |
| | CCCGGATTTCGGTGATTTCAACCGGACCTACACTTCAGGCGAGGCGAAGGCAA | CLARSVLSKAKILLLDEPSAHLDPVTYQIIRRT |
| | TCCGTGCTCAACCTCATGACGCATTCGGTAAACCAGGGGCAAAACATTCACCG | LKQAFADCTVILCEHRIEAMLECQQFLVIEEN |
| | CAAAACGACGGCCTCAACGAGAAAAGTGTCACTTGCACCCCAGGCGAATTTGA | KVRQYDSIQKLLNERSLFRQAISPSDRVKLFP |
| | CTGAACTCGACATCTACAGCCGTAGGCTTTCGCAAGAAACCGGACTTGAGATC | HRNSSKCKSKPQIAALKEETEEEVQDTRL |
| | AGCGAAGAAATCAATGAAGAAGATTTGAAAGAGTGTTTCTTTGATGACATGGAA | (SEQ ID NO: 258) |
| | TCAATCCCAGCGGTGACAACGTGGAACACATACTTGCGTTACATCACGGTGCA | |
| | CAAGTCCTTGATTTTCGTCCTCATCTGGTGTCTCGTGATCTTTCTCGCTGAGGT | |
| | CGCAGCGTCACTTGTGGTCCTCTGGCTGCTTGGTAATACGCCCTTGCAAGACA | |
| | AAGGCAATTCTACACACTCAAGAAACAATTCCTATGCCGTGATTATCACTTCTAC | |
| | AAGCTCGTATTACGTGTTTTACATCTACGTAGGAGTGGCCGACACTCTGCTCGC | |
| | GATGGGTTTCTTCCGAGGACTCCCACTCGTTCACACGCTTATCACTGTCTCCAA | |
| | GATTCTCCACCATAAGATGCTTCATAGCGTACTGCAGGCTCCCATGTCCACCTT | |
| | GAATACGCTCAAGGCGGGAGGTATTTTGAATCGCTTCTCAAAAGATATTGCAAT | |
| | TTTGGATGACCTTCTGCCCCTGACGATCTTCGACTTCATCCAGTTGTTGCTGAT | |
| | CGTGATTGGGGCTATTGCAGTAGTCGCTGTCCTCCAGCCTTACATTTTTGTCGC | |
| | GACCGTTCCGGTGATCGTGGCGTTTATCATGCTGCGGGCCTATTTCTTGCAGA | |
| | CGTCACAGCAGCTTAAGCAACTGGAGTCTGAAGGGAGGTCGCCTATCTTTACG | |
| | CATCTTGTGACCAGTTTGAAGGGATTGTGGACGTTGCGCGCCTTTGGCAGGCA | |
| | GCCCTACTTTGAAACACTGTTCCACAAAGCGCTGAATCTCCATACGGCAAATTG | |
| | GTTTTTGTATTTGAGTACCCTCCGATGGTTTCAGATGCGCATTGAGATGATTTTT | |
| | GTGATCTTCTTTATCGCGGTGACTTTTATCTCCATCTTGACCACGGGAGAGGGC | |
| | GAGGGACGGGTCGGTATTATCCTGACACTCGCCATGAACATTATGAGCACTTT | |
| | GCAGTGGGCAGTGAACAGCTCGATTGATGTGGATAGCCTGATGAGGTCCGTTT | |
| | CGAGGGTCTTTAAGTTCATCGACATGCCGACGGAGGGAAAGCCCACAAAAAGT | |
| | ACGAAACCCTATAAGAATGGGCAATTGAGTAAGGTAATGATCATCGAGAACAGT | |
| | CACGTGAAGAAGGATGACATCTGGCCTAGCGGGGGTCAGATGACCGTGAAGG | |
| | ACCTGACGGCAAAATACACCGAGGGAGGGAACGCAATCCTTGAAAACATCTCG | |
| | TTCAGCATTAGCCCCGGTCAGCGTGTGGGGTTGCTCGGGAGGACCGGGTCAG | |
| | GAAAATCGACGTTGCTGTCGGCCTTCTTGAGACTTCTGAATACAGAGGGTGAG | |
| | ATCCAGATCGACGGCGTTTCGTGGGATAGCATCACCTTGCAGCAGTGGCGGAA | |
| | AGCGTTTGGAGTAATCCCCCAAAAGGTCTTTATCTTTAGCGGAACCTTCCGAAA | |
| | GAATCTCGATCCTTATGAACAGTGGTCAGATCAAGAGATTTGGAAAGTCGCGGA | |
| | CGAGGTTGGCCTTCGGAGTGTAATCGAGCAGTTTCCGGGAAAACTCGACTTTG | |
| | TCCTTGTAGATGGGGGATGCGTCCTGTCGCATGGGCACAAGCAGCTCATGTGC | |
| | CTGGCGCGATCCGTCCTCTCTAAAGCGAAAATTCTTCTCTTGGATGAACCTTCG | |
| | GCCCATCTGGACCCGGTAACGTATCAGATCATCAGAAGGACACTTAAGCAGGC | |
| | GTTTGCCGACTGCACGGTGATTCTCTGTGAGCATCGTATCGAGGCCATGCTCG | |
| | AATGCCAGCAATTTCTTGTCATCGAAGAGAATAAGGTCCGCCAGTACGACTCCA | |
| | TCCAGAAGCTGCTTAATGAGAGATCATTGTTCCGGCAGGCGATTTCACCATCCG | |
| | ATAGGGTGAAACTTTTTCCACACAGAAATTCGTCGAAGTGCAAGTCCAAACCGC | |
| | AGATCGCGGCCTTGAAAGAAGAGACTGAAGAAGAAGTTCAAGACACGCGTCTT | |
| | TGATAA (SEQ ID NO: 267) | |
| N/A | ATGCAGCGGAGCCCCCTGGAGAAGGCCAGCGTGGTCAGCAAACTGTTTTTCAG | MQRSPLEKASVVSKLFFSWTRPILRKGYRQR |
| | CTGGACCCGGCCCATCCTGCGGAAGGGCTATCGGCAACGGCTGGAGCTGAGC | LELSDIYQIPSVDSADNLSEKLEREWDRELAS |
| | GACATCTACCAGATCCCCAGCGTGGACAGCGCCGACAACCTGAGCGAGAAGC | KKNPKLINALRRCFFWRFMFYGIFLYLGEVTK |
| | TGGAGCGGGAGTGGGACCGGGAGCTGGCCAGCAAGAAAAACCCCAAGCTGAT | AVQPLLLGRIIASYDPDNKEERSIAIYLGIGLCL |
| | CAACGCCCTGCGGAGGTGCTTCTTCTGGCGGTTCATGTTCTACGGCATCTTTCT | LFIVRTLLLHPAIFGLHHIGMQMRIAMFSLIYKK |
| | GTATCTGGGCGAGGTGACCAAGGCCGTGCAGCCTCTGCTGCTGGGACGGATT | TLKLSSRVLDKISIGQLVSLLSNNLNKFDEGLA |
| | ATTGCCAGCTACGACCCCGACAACAAGGAGGAACGGAGTATTGCCATCTACCT | LAHFVWIAPLQVALLMGLIWELLQASAFCGLG |
| | GGGCATCGGCCTGTGCCTGTTGTTCATCGTGCGGACCCTGCTGCTGCACCCC | FLIVLALFQAGLGRMMMKYRDQRAGKISERL |
| | GCCATCTTCGGCCTGCACCATATCGGCATGCAAATGCGGATTGCCATGTTCAG | VITSEMIENIQSVKAYCWEEAMEKMIENLRQT |
| | CCTGATCTACAAGAAAACCCTCAAGCTGAGCTCTCGGGTCCTGGACAAGATCA | ELKLTRKAAYVRYFNSSAFFFSGFFVVFLSVL |
| | GCATCGGCCAGCTGGTGAGCCTGCTCAGCAACAATCTGAACAAGTTCGACGAG | PYALIKGIILRKIFTTISFCIVLRMAVTRQFPWA |
| | GGCCTGGCCCTGGCCCACTTCGTGTGGATCGCTCCCCTCCAGGTGGCTCTGC | VQTWYDSLGAINKIQDFLQKQEYKTLEYNLTT |
| | TCATGGGCCTGATCTGGGAGCTGTTGCAGGCTAGCGCTTTCTGCGGCCTGGG | TEVVMENVTAFWEEGFGELFEKAKQNNNNR |
| | CTTTCTGATTGTGCTGGCCCTGTTTCAGGCTGGCCTCGGCCGGATGATGATGA | KTSNGDDSLFFSNFSLLGTPVLKDINFKIERG |
| | AGTACCGGGACCAGCGGGCCGGCAAAATCAGTGAGCGCCTGGTCATCACAAG | QLLAVAGSTGAGKTSLLMVIMGELEPSEGKIK |
| | CGAAATGATTGAGAATATCCAGAGCGTGAAGGCCTACTGTTGGGAAGAGGCCA | HSGRISFCSQFSWIMPGTIKENIIFGVSYDEYR |
| | TGGAGAAGATGATCGAGAACCTGCGGCAGACCGAACTGAAACTGACCCGGAA | YRSVIKACQLEEDISKFAEKDNIVLGEGGITLS |
| | GGCCGCTTACGTGCGGTACTTCAACAGCAGCGCCTTCTTTTTCAGTGGCTTCTT | GGQRARISLARAVYKDADLYLLDSPFGYLDVL |
| | CGTGGTGTTCCTGAGCGTGCTGCCCTACGCCCTGATCAAAGGCATTATCCTCC | TEKEIFESCVCKLMANKTRILVTSKMEHLKKA |
| | GGAAGATCTTCACCACAATTAGCTTTTGCATCGTGCTGCGGATGGCCGTGACC | DKILILHEGSSYFYGTFSELQNLQPDFSSKLM |
| | CGGCAGTTCCCCTGGGCCGTGCAGACCTGGTACGACAGCCTGGGCGCCATCA | GCDSFDQFSAERRNSILTETLHRFSLEGDAP |
| | ACAAGATCCAGGATTTCCTCCAGAAGCAGGAGTACAAGACCCTGGAGTACAAC | VSWTETKKQSFKQTGEFGEKRKNSILNPINSI |
| | CTGACCACCACTGAGGTGGTTATGGAGAACGTGACCGCCTTCTGGGAGGAGG | RKFSIVQKTPLQMNGIEEDSDEPLERRLSLVP |
| | GCTTCGGCGAGCTGTTCGAGAAGGCCAAGCAGAACAACAACAATCGGAAAACC | DSEQGEAILPRISVISTGPTLQARRRQSVLNL |
| | AGCAACGGCGACGATAGCCTGTTCTTTAGCAACTTCAGCCTGCTGGGCACCCC | MTHSVNQGQNIHRKTTASTRKVSLAPQANLT |
| | CGTGCTGAAGGACATCAACTTCAAGATCGAGCGGGGCCAGCTGCTCGCCGTG | ELDIYSRRLSQETGLEISEEINEEDLKECFFDD |
| | GCCGGCAGCACCGGCGCCGGCAAGACCAGCCTGCTGATGGTGATCATGGGCG | MESIPAVTTWNTYLRYITVHKSLIFVLIWCLVIF |
| | AGCTGGAGCCCAGCGAGGGCAAGATCAAGCACAGCGGCCGGATCAGCTTCTG | LAEVAASLVVLWLLGNTPLQDKGNSTHSRNN |
| | CAGCCAGTTCAGCTGGATCATGCCCGGCACCATCAAAGAGAATATCATATTTGG | SYAVIITSTSSYYVFYIYVGVADTLLAMGFFRG |
| | CGTCAGCTACGACGAGTACCGGTACCGCAGCGTCATCAAGGCCTGCCAGCTG | LPLVHTLITVSKILHHKMLHSVLQAPMSTLNTL |
| | GAGGAGGACATCAGCAAGTTCGCCGAGAAGGACAACATCGTGCTGGGCGAGG | KAGGILNRFSKDIAILDDLLPLTIFDFIQLLLIVIG |
| | GCGGAATCACCCTGAGTGGCGGACAGCGCGCCCGCATCAGTCTGGCTCGGGC | AIAVVAVLQPYIFVATVPVIVAFIMLRAYFLQTS |
| | CGTGTACAAGGACGCCGACCTGTACCTGCTGGACAGCCCCTTCGGCTACCTGG | QQLKQLESEGRSPIFTHLVTSLKGLWTLRAF |
| | ACGTGCTGACCGAGAAGGAGATCTTCGAGAGCTGCGTGTGTAAGCTCATGGCC | GRQPYFETLFHKALNLHTANWFLYLSTLRWF |
| | AACAAGACCCGGATTCTCGTCACAAGTAAGATGGAGCACCTGAAGAAGGCCGA | QMRIEMIFVIFFIAVTFISILTTGEGEGRVGIILT |
| | CAAGATCCTGATCCTGCACGAGGGCAGCTCTTACTTCTACGGCACCTTCAGCG | LAMNIMSTLQWAVNSSIDVDSLMRSVSRVFK |
| | AGCTGCAGAACCTGCAGCCCGACTTCAGCAGCAAGCTGATGGGCTGCGACAG | FIDMPTEGKPTKSTKPYKNGQLSKVMIIENSH |
| | CTTCGACCAGTTCAGCGCCGAGCGGCGCAACAGTATCCTCACCGAAACACTGC | VKKDDIWPSGGQMTVKDLTAKYTEGGNAILE |
| | ATCGGTTTAGCCTGGAGGGCGACGCCCCCGTGAGCTGGACCGAAACAAAGAA | NISFSISPGQRVGLLGRTGSGKSTLLSAFLRL |
| | GCAGAGCTTCAAGCAGACCGGCGAGTTCGGCGAGAAGCGGAAGAATAGCATT | LNTEGEIQIDGVSWDSITLQQWRKAFGVIPQK |
| | CTGAACCCCATCAACAGCATCCGGAAGTTCAGCATCGTGCAGAAGACACCCCT | VFIFSGTFRKNLDPYEQWSDQEIWKVADEVG |
| | CCAGATGAACGGCATCGAGGAGGACAGCGACGAGCCCCTGGAGCGGAGGCT | LRSVIEQFPGKLDFVLVDGGCVLSHGHKQLM |
| | GAGCCTGGTGCCCGACAGCGAGCAGGGCGAGGCCATCCTGCCCCGGATCAG | CLARSVLSKAKILLLDEPSAHLDPVTYQIIRRT |
| | CGTGATCAGCACCGGCCCCACCCTGCAGGCCCGGGGGGGGCAAAGCGTCCTG | LKQAFADCTVILCEHRIEAMLECQQFLVIEEN |
| | AACCTGATGACCCACAGCGTGAACCAGGGCCAGAACATCCACCGGAAGACCAC | KVRQYDSIQKLLNERSLFRQAISPSDRVKLFP |
| | CGCCAGCACCCGGAAGGTGAGCCTGGCCCCCCAGGCCAACCTGACCGAGCTG | HRNSSKCKSKPQIAALKEETEEEVQDTRL |
| | GACATCTACAGCCGGAGACTGAGCCAGGAAACAGGCCTGGAGATCAGCGAGG | (SEQ ID NO: 258) |
| | AAATCAACGAGGAGGACCTGAAGGAGTGCTTCTTCGACGACATGGAGAGCATC | |
| | CCCGCCGTGACCACCTGGAACACCTACCTGCGGTACATCACCGTGCACAAGAG | |
| | CCTCATCTTTGTGCTGATCTGGTGCCTGGTGATCTTCCTGGCCGAGGTGGCCG | |
| | CCAGCCTGGTGGTGCTGTGGCTGTTGGGCAACACCCCCCTGCAGGACAAGGG | |
| | CAACAGCACCCACAGCCGGAACAACAGCTACGCCGTCATCATTACCAGCACCA | |
| | GCAGCTACTACGTGTTCTACATCTACGTGGGCGTGGCCGACACCCTGCTGGCC | |
| | ATGGGCTTCTTCCGGGGCCTGCCCCTGGTGCACACCCTGATCACCGTGAGTAA | |
| | GATTCTGCACCACAAGATGCTGCACAGCGTGCTGCAGGCCCCTATGAGTACCC | |
| | TCAACACCCTGAAGGCCGGCGGGATCCTCAACCGCTTCAGCAAGGATATTGCC | |
| | ATTCTGGACGACCTGCTCCCCCTGACCATCTTCGACTTCATCCAGCTGCTCCTG | |
| | ATCGTGATCGGCGCTATTGCCGTGGTGGCCGTGCTGCAGCCCTACATCTTCGT | |
| | GGCCACCGTGCCCGTGATCGTGGCCTTCATCATGCTGCGGGCCTACTTCCTGC | |
| | AGACCAGCCAGCAACTGAAGCAGCTGGAGAGTGAGGGACGGAGCCCCATCTT | |
| | CACCCACCTGGTGACCAGCCTGAAGGGCCTGTGGACCCTGCGGGCCTTCGGC | |
| | CGGCAGCCCTACTTCGAAACACTGTTCCACAAGGCCCTGAACCTGCACACCGC | |
| | CAACTGGTTCCTGTACCTCAGCACACTGCGGTGGTTCCAGATGCGGATCGAGA | |
| | TGATCTTCGTGATCTTCTTTATTGCCGTGACCTTCATCAGCATCCTGACCACAG | |
| | GCGAGGGCGAGGGCCGGGTGGGCATCATCCTGACCCTGGCCATGAACATCAT | |
| | GAGCACACTGCAGTGGGCCGTGAACAGCTCCATCGACGTGGACAGCCTGATG | |
| | CGGAGTGTGAGCCGGGTGTTCAAGTTCATCGACATGCCCACCGAGGGCAAGC | |
| | CCACCAAGAGCACCAAGCCCTACAAGAACGGCCAACTGAGTAAGGTGATGATC | |
| | ATTGAGAACAGCCACGTGAAGAAGGACGACATCTGGCCCAGCGGCGGCCAGA | |
| | TGACCGTGAAGGACCTGACCGCCAAGTATACCGAAGGGGCAACGCCATCCT | |
| | GGAGAACATCAGCTTCAGCATCAGCCCCGGCCAGCGGGTGGGCCTGTTGGGC | |
| | CGGACCGGCAGCGGCAAGAGCACCCTGCTGAGCGCCTTCCTGCGGCTGCTGA | |
| | ACACCGAGGGCGAGATCCAGATCGACGGCGTGAGCTGGGACAGCATCACCCT | |
| | GCAGCAGTGGCGGAAGGCCTTCGGCGTGATCCCCCAGAAGGTGTTCATCTTCA | |
| | GCGGCACCTTCCGGAAGAATCTGGATCCCTACGAGCAGTGGAGCGACCAGGA | |
| | GATCTGGAAGGTGGCCGACGAGGTGGGCCTGCGGAGCGTGATCGAGCAGTTC | |
| | CCCGGCAAGCTGGACTTCGTGCTGGTGGACGGCGGCTGTGTGCTCAGCCACG | |
| | GCCACAAGCAGCTGATGTGCCTGGCCCGGAGCGTGCTGAGCAAGGCCAAGAT | |
| | CCTGCTCCTGGACGAGCCCAGCGCCCACCTGGACCCCGTGACCTACCAGATC | |
| | ATCCGGCGGACCCTGAAGCAGGCCTTCGCCGACTGCACCGTGATCCTGTGCG | |
| | AGCACCGGATCGAGGCCATGCTGGAGTGCCAGCAGTTCCTGGTGATCGAGGA | |
| | GAACAAGGTGCGGCAGTACGACAGCATCCAGAAGCTGTTGAACGAGCGGAGC | |
| | CTGTTCCGGCAGGCCATCAGCCCCAGCGACCGGGTGAAGCTGTTCCCCCACC | |
| | GGAACAGCAGCAAGTGCAAGAGCAAGCCCCAGATCGCCGCACTGAAGGAGGA | |
| | AACAGAGGAAGAGGTGCAGGACACCCGGCTG (SEQ ID NO: 268) | |

In some embodiments, the circular polyribonucleotide comprises one or more expression sequences (coding sequences) and is configured for persistent expression in a cell of a subject in vivo. In some embodiments, the circular polyribonucleotide is configured such that expression of the one or more expression sequences in the cell at a later time point is equal to or higher than an earlier time point. In such embodiments, the expression of the one or more expression sequences may be either maintained at a relatively stable level or may increase over time. The expression of the expression sequences may be relatively stable for an extended period of time. For instance, in some cases, the expression of the one or more expression sequences in the cell over a time period of at least 7, 8, 9, 10, 12, 14, 16, 18, 20, 22, 23 or more days does not decrease by 50%, 45%, 40%, 35%, 30%, 25%, 20%, 15%, 10%, or 5%. In some cases, in some cases, the expression of the one or more expression sequences in the cell is maintained at a level that does not vary by more than 50%, 45%, 40%, 35%, 30%, 25%, 20%, 15%, 10%, or 5% for at least 7, 8, 9, 10, 12, 14, 16, 18, 20, 22, 23 or more days.

### Plant-Modifying Polypeptides

In some embodiments, the polyribonucleotide described herein (e.g., the polyribonucleotide cargo of the polyribonucleotide) includes at least one expression sequence encoding a plant-modifying polypeptide. A plant-modifying polypeptide refers to a polypeptide that can alter the genetic properties (e.g., increase gene expression, decrease gene expression, or otherwise alter the nucleotide sequence of DNA or RNA), epigenetic properties, or physiological or biochemical properties of a plant in a manner that results in a change in the plant's physiology or phenotype, e.g., an increase or decrease in the plant's fitness. In some embodiments, the polyribonucleotide encodes two, three, four, five, six, seven, eight, nine, ten or more different plant-modifying polypeptides, or multiple copies of one or more plant-modifying polypeptides. A plant-modifying polypeptide may change the physiology or phenotype of, or increase or decrease the fitness of, a variety of plants, or can be one that affects such change(s) in one or more specific plants (e.g., a specific species or genera of plants).

Examples of polypeptides that can be used herein can include an enzyme (e.g., a metabolic recombinase, a helicase, an integrase, a RNAse, a DNAse, or a ubiquitination protein), a pore-forming protein, a signaling ligand, a cell penetrating peptide, a transcription factor, a receptor, an antibody, a nanobody, a gene editing protein (e.g., CRISPR-Cas endonuclease, TALEN, or zinc finger), riboprotein, a protein aptamer, or a chaperone.

### Agricultural Polypeptides

In some embodiments, the polyribonucleotide described herein (e.g., the polyribonucleotide cargo of the polyribonucleotide) includes at least one expression sequence encoding an agricultural polypeptide. An agricultural polypeptide is a polypeptide that is suitable for an agricultural use. In embodiments, an agricultural polypeptide is applied to a plant or seed (e.g., by foliar spray, dusting, injection, or seed coating) or to the plant's environment (e.g., by soil drench or granular soil application), resulting in an alteration of the plant's physiology, phenotype, or fitness. Embodiments of an agricultural polypeptide include polypeptides that alter a level, activity, or metabolism of one or more microorganisms resident in or on a plant or non-human animal host, the alteration resulting in an increase in the host's fitness. In some embodiments the agricultural polypeptide is a plant polypeptide. In some embodiments, the agricultural polypeptide is an insect polypeptide. In some embodiments, the agricultural polypeptide has a biological effect when contacted with a non-human vertebrate animal, invertebrate animal, microbial, or plant cell.

In some embodiments, the polyribonucleotide encodes two, three, four, five, six, seven, eight, nine, ten or more agricultural polypeptides, or multiple copies of one or more agricultural polypeptides.

Embodiments of polypeptides useful in agricultural applications include, for example, bacteriocins, lysins, antimicrobial peptides, nodule C-rich peptides, and bacteriocyte regulatory peptides. Such polypeptides can be used to alter the level, activity, or metabolism of target microorganisms for increasing the fitness of insects, such as honeybees and silkworms. Embodiments of agriculturally useful polypeptides include peptide toxins, such as those naturally produced by entomopathogenic bacteria (e.g., Bacillus thuringiensis, Photorhabdus luminescens, Serratia entomophila, or Xenorhabdus nematophila), as is known in the art. Embodiments of agriculturally useful polypeptides include polypeptides (including small peptides such as cyclodipeptides or diketopiperazines) for controlling agriculturally important pests or pathogens, e.g., antimicrobial polypeptides or antifungal polypeptides for controlling diseases in plants, or pesticidal polypeptides (e.g., insecticidal polypeptides or nematicidal polypeptides) for controlling invertebrate pests such as insects or nematodes. Embodiments of agriculturally useful polypeptides include antibodies, nanobodies, and fragments thereof, e.g., antibody or nanobody fragments that retain at least some (e.g., at least 10%) of the specific binding activity of the intact antibody or nanobody. Embodiments of agriculturally useful polypeptides include transcription factors, e.g., plant transcription factors; see, e.g., the “AtTFDB” database listing the transcription factor families identified in the model plant Arabidopsis thaliana), publicly available at agris-knowledgebase.org/AtTFDB/. Embodiments of agriculturally useful polypeptides include nucleases, for example, exonucleases or endonucleases (e.g., Cas nucleases such as Cas9 or Cas12a). Embodiments of agriculturally useful polypeptides further include cell-penetrating peptides, enzymes (e.g., amylases, cellulases, peptidases, lipases, chitinases), peptide pheromones (for example, yeast mating pheromones, invertebrate reproductive and larval signaling pheromones, see, e.g., Altstein (2004) Peptides, 25:1373-1376).

### Termination Elements

In some embodiments, the polyribonucleotide described herein includes at least one termination element. In some embodiments, the polyribonucleotide includes a termination element operably linked to an expression sequence. In some embodiments, the polynucleotide lacks a termination element.

In some embodiments, the polyribonucleotide includes one or more expression sequences, and each expression sequence may or may not have a termination element. In some embodiments, the polyribonucleotide includes one or more expression sequences, and the expression sequences lack a termination element, such that the polyribonucleotide is continuously translated. Exclusion of a termination element may result in rolling circle translation or continuous expression of expression product.

In some embodiments, the circular polyribonucleotide includes one or more expression sequences, and each expression sequence may or may not have a termination element. In some embodiments, the circular polyribonucleotide includes one or more expression sequences, and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide includes a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences includes two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation (e.g., UAA, UGA, UAG). In some embodiments, one or more termination elements in the circular

polyribonucleotide are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell. In some embodiments, the termination element is a stop codon.

In some embodiments, an expression sequence includes a poly-A sequence (e.g., at the 3′ end of an expression sequence, for example 3′ to a termination element). In some embodiments, the length of a poly-A sequence is greater than 10 nucleotides in length. In one embodiment, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is designed according to the descriptions of the poly-A sequence in [0202]-[0204] of International Patent Publication No. WO2019/118919A1, which is incorporated herein by reference in its entirety. In some embodiments, the expression sequence lacks a poly-A sequence (e.g., at the 3′ end of an expression sequence).

In some embodiments, a circular polyribonucleotide includes a polyA, lacks a polyA, or has a modified polyA to modulate one or more characteristics of the circular polyribonucleotide. In some embodiments, the circular polyribonucleotide lacking a polyA or having modified polyA improves one or more functional characteristics, e.g., immunogenicity (e.g., the level of one or more marker of an immune or inflammatory response), half-life, and/or expression efficiency.

Further examples of termination elements are described in paragraphs [0169]-[0170] of International Patent Publication No. WO2019/118919, which is hereby incorporated by reference in its entirety.

### Modifications

A polyribonucleotide may include one or more substitutions, insertions and/or additions, deletions, and covalent modifications with respect to reference sequences, in particular, the parent polyribonucleotide, are included within the scope of this disclosure.

In some embodiments, a polyribonucleotide includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27:196-197). In some embodiments, the first isolated nucleic acid includes messenger RNA (mRNA). In some embodiments, the polyribonucleotide includes at least one nucleoside selected from the group such as those described in of International Patent Publication No. WO2019/118919A1, which is incorporated herein by reference in its entirety.

A polyribonucleotide may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, a polyribonucleotide includes at least one N (6)methyladenosine (m6A) modification to increase translation efficiency. In some embodiments, the m6A modification can reduce immunogenicity (e.g., reduce the level of one or more marker of an immune or inflammatory response) of the polyribonucleotide.

In some embodiments, a modification may include a chemical or cellular induced modification. For example, some non-limiting examples of intracellular RNA modifications are described by Lewis and Pan in “RNA modifications and structures cooperate to guide RNA-protein interactions” from Nat Reviews Mol Cell Biol, 2017, 18:202-210.

In some embodiments, chemical modifications to the ribonucleotides of a polyribonucleotide may enhance immune evasion. The polyribonucleotide may be synthesized and/or modified by methods well established in the art, such as those described in Current Protocols in Nucleic Acid Chemistry, Beaucage, S. L. et al. (Eds.), John Wiley & Sons, Inc., New York, NY, USA, which is hereby incorporated herein by reference. Modifications include, for example, end modifications, e.g., 5′ end modifications (phosphorylation (mono-, di- and tri-), conjugation, inverted linkages, etc.), 3′ end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified ribonucleotide bases may also include 5-methylcytidine and pseudouridine. In some embodiments, base modifications may modulate expression, immune response, stability, subcellular localization, to name a few functional effects, of the polyribonucleotide. In some embodiments, the modification includes a bi-orthogonal nucleotide, e.g., an unnatural base. See for example, Kimoto et al, Chem Commun (Camb), 2017, 53:12309, DOI: 10.1039/c7cc06661a, which is hereby incorporated by reference.

In some embodiments, sugar modifications (e.g., at the 2′ position or 4′ position) or replacement of the sugar one or more ribonucleotides of the polyribonucleotide may, as well as backbone modifications, include modification or replacement of the phosphodiester linkages. Specific examples of polyribonucleotide include, but are not limited to, polyribonucleotide including modified backbones or no natural internucleoside linkages such as internucleoside modifications, including modification or replacement of the phosphodiester linkages. Polyribonucleotides having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, the polyribonucleotide will include ribonucleotides with a phosphorus atom in its internucleoside backbone.

Modified polyribonucleotide backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the polyribonucleotide may be negatively or positively charged.

The modified nucleotides, which may be incorporated into the polyribonucleotide, can be modified at the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylenephosphonates).

The a-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the polyribonucleotide is expected to reduce the innate immune response through weaker binding/activation of cellular innate immune molecules.

In specific embodiments, a modified nucleoside includes an alpha-thio-nucleoside (e.g., 5′-0-(I-thiophosphate)-adenosine, 5′-0-(I-thiophosphate)-cytidine (a-thio-cytidine), 5′-0-(I-thiophosphate)-guanosine, 5′-0-(I-thiophosphate)-uridine, or 5′-0-(1-thiophosphate)-pseudouridine).

Other internucleoside linkages that may be employed according to the present disclosure, including internucleoside linkages which do not contain a phosphorous atom, are described herein. In some embodiments, a polyribonucleotide may include one or more cytotoxic nucleosides. For example, cytotoxic nucleosides may be incorporated into polyribonucleotide, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, I-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-I-(tetrahydrofuran-2-yl)pyrimidine-2,4 (1H,3H)-dione), troxacitabine, tezacitabine, 2′-deoxy-2′-methylidenecytidine (DMDC), and 6-mercaptopurine. Additional examples include fludarabine phosphate, N4-behenoyl-I-beta-D-arabinofuranosylcytosine, N4-octadecyl-1-beta-D-arabinofuranosylcytosine, N4-palmitoyl-I-(2-C-cyano-2-deoxy-beta-D-arabino-pentofuranosyl) cytosine, and P-4055 (cytarabine 5′-elaidic acid ester).

A polyribonucleotide may or may not be uniformly modified along the entire length of the molecule. For example, one or more or all types of nucleotides (e.g., naturally occurring nucleotides, purine or pyrimidine, or any one or more or all of A, G, U, C, I, pU) may or may not be uniformly modified in the polyribonucleotide, or in a given predetermined sequence region thereof. In some embodiments, the polyribonucleotide includes a pseudouridine. In some embodiments, the polyribonucleotide includes an inosine, which may aid in the immune system characterizing the polyribonucleotide as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

In some embodiments, all nucleotides in a polyribonucleotide (or in a given sequence region thereof) are modified. In some embodiments, the modification may include an m6A, which may augment expression; an inosine, which may attenuate an immune response; pseudouridine, which may increase RNA stability, or translational readthrough (stagger element), an m5C, which may increase stability; and a 2,2,7-trimethylguanosine, which aids subcellular translocation (e.g., nuclear localization).

Different sugar modifications, nucleotide modifications, and/or internucleoside linkages (e.g., backbone structures) may exist at various positions in a polyribonucleotide. One of ordinary skill in the art will appreciate that the nucleotide analogs or other modification(s) may be located at any position(s) of the polyribonucleotide, such that the function of the polyribonucleotide is not substantially decreased. A modification may also be a non-coding region modification. The polyribonucleotide may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

### Production Methods

The disclosure provides methods for producing circular polyribonucleotides, including, e.g., recombinant technology or chemical synthesis. For example, a DNA molecule used to produce an RNA circle can include a DNA sequence of a naturally occurring nucleic acid sequence, a modified version thereof, or a DNA sequence encoding a synthetic polypeptide not normally found in nature (e.g., chimeric molecules or fusion proteins). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification or mutagenesis of selected regions of a nucleic acid sequence, synthesis of oligonucleotide mixtures and ligation of mixture groups to “build” a mixture of nucleic acid molecules and combinations thereof.

The circular polyribonucleotides may be prepared according to any available technique, including, but not limited to chemical synthesis and enzymatic synthesis. In some embodiments, a linear primary construct or linear polyribonucleotide for circularization may be cyclized or concatenated to create a circRNA described herein. In some embodiments, the linear polyribonucleotide for circularization may be cyclized in vitro prior to formulation and/or delivery. In some embodiments, the circular polyribonucleotide may be in a mixture with linear polyribonucleotides. In some embodiments, the linear polyribonucleotides have the same nucleic acid sequence as the circular polyribonucleotides.

The mechanism of cyclization or concatenation may occur through methods such as, e.g., chemical, enzymatic, splint ligation, or ribozyme-catalyzed methods. The newly formed 5′-3′ linkage may be an intramolecular linkage or an intermolecular linkage. For example, a splint ligase, such as a SplintR® ligase, can be used for splint ligation. According to this method, a single stranded polynucleotide (splint), such as a single-stranded DNA or RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circRNA. In some embodiments, a DNA or RNA ligase may be used in the synthesis of the circular polynucleotides. As a non-limiting example, the ligase may be a circ ligase or circular ligase.

In some embodiments, either the 5′ or 3′ end of the linear polyribonucleotide can encode a ligase ribozyme sequence such that during in vitro transcription, the resultant linear polyribonucleotide for circularization includes an active ribozyme sequence capable of ligating the 5′ end of the linear polyribonucleotide for circularization to the 3′ end of the linear polyribonucleotide for circularization. The ligase ribozyme may be derived from the Group I Intron, Hepatitis Delta Virus, Hairpin ribozyme or may be selected by SELEX (systematic evolution of ligands by exponential enrichment).

In another example, a linear polyribonucleotide may be cyclized or concatenated by using at least one non-nucleic acid moiety. In one aspect, the at least one non-nucleic acid moiety may react with regions or features near the 5′ terminus or near the 3′ terminus of the linear polyribonucleotide for circularization in order to cyclize or concatenate the linear polyribonucleotide for circularization. In another aspect, the at least one non-nucleic acid moiety may be located in or linked to or near the 5′ terminus or the 3′ terminus of the linear polyribonucleotide for circularization. The non-nucleic acid moieties may be homologous or heterologous. As a non-limiting example, the non-nucleic acid moiety may be a linkage such as a hydrophobic linkage, ionic linkage, a biodegradable linkage, or a cleavable linkage. As another non-limiting example, the non-nucleic acid moiety is a ligation moiety.

As yet another non-limiting example, the non-nucleic acid moiety may be an oligonucleotide or a peptide moiety, such as an aptamer or a non-nucleic acid linker as described herein.

In some embodiments, a linear polyribonucleotide for circularization may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). In some embodiments, the 5′ end of at least a portion of the linear polyribonucleotides includes a monophosphate moiety. In some embodiments, a population of polyribonucleotides including circular and linear polyribonucleotides is contacted with RppH prior to digesting at least a portion of the linear polyribonucleotides with a 5′ exonuclease and/or a 3′ exonuclease. Alternately, converting the 5′ triphosphate of the linear polyribonucleotide for circularization into a 5′ monophosphate may occur by a two-step reaction including: (a) contacting the 5′ nucleotide of the linear polyribonucleotide for circularization with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate. In another aspect, linear polyribonucleotides for circularization may be cyclized or concatenated by self-splicing. In some embodiments, the linear polyribonucleotide may include a sequence that mediates self-ligation. In some embodiments, the linear polyribonucleotides may include loop E sequence (e.g., in PSTVd) to self-ligate. In some embodiments, the linear polyribonucleotide may include a HDV sequence, e.g., HDV replication domain conserved sequence, GGCUCAUCUCGACAAGAGGCGGCAGUCCUCAGUACUCUUACUCUUUUCUGUAAAGAGGAGAC UGCUGGACUCGCCGCCCAAGUUCGAGCAUGAGCC (SEQ ID NO: 271) (Beeharry et al 2004) or GGCUAGAGGCGGCAGUCCUCAGUACUCUUACUCUUUUCUGUAAAGAGGAGACUGCUGGACUC GCCGCCCGAGCC (SEQ ID NO: 272), to self-ligate. In another embodiment, the linear polyribonucleotides may include a self-circularizing intron, e.g., a 5′ and 3′ slice junction, or a self-circularizing catalytic intron such as a Group I, Group II, or Group III Introns. Nonlimiting examples of group I intron self-splicing sequences may include self-splicing permuted intron-exon sequences derived from T4 bacteriophage gene td, and the intervening sequence (IVS) rRNA of Tetrahymena, cyanobacterium Anabaena pre-tRNA-Leu gene, or a Tetrahymena pre-rRNA.

In some embodiments, the polyribonucleotide includes catalytic intron fragments, such as a 3′ half of Group I catalytic intron fragment and a 5′ half of Group I catalytic intron fragment. The first and second annealing regions may be positioned within the catalytic intron fragments. Group I catalytic introns are self-splicing ribozymes that catalyze their own excision from mRNA, tRNA, and rRNA precursors via two-metal ion phosphoryl transfer mechanism. Importantly, the RNA itself self-catalyzes the intron removal without the requirement of an exogenous enzyme, such as a ligase.

In some embodiments, the 3′ half of Group I catalytic intron fragment and the 5′ half of Group I catalytic intron fragment are from a cyanobacterium Anabaena pre-tRNA-Leu gene, or a Tetrahymena pre-rRNA.

In some embodiments, the 3′ half of Group I catalytic intron fragment and the 5′ half of Group I catalytic intron fragment are from a Cyanobacterium Anabaena pre-tRNA-Leu gene, and the 3′ exon fragment includes the first annealing region and the 5′ exon fragment includes the second annealing region. The first annealing region may include, e.g., from 5 to 50, e.g., from 10 to 15 (e.g., 10, 11, 12, 13, 14, or 15) ribonucleotides and the second annealing region may include, e.g., from 5 to 50, e.g., from 10 to 15 (e.g., 10, 11, 12, 13, 14, or 15) ribonucleotides.

In some embodiments, the 3′ half of Group I catalytic intron fragment and the 5′ half of Group I catalytic intron fragment are from a Tetrahymena pre-rRNA, and the 3′ half of Group I catalytic intron fragment includes the first annealing region and the 5′ exon fragment includes the second annealing region. In some embodiments, the 3′ exon includes the first annealing region and the 5′ half of Group I catalytic intron fragment includes the second annealing region. The first annealing region may include, e.g., from 6 to 50, e.g., from 10 to 16 (e.g., 10, 11, 12, 13, 14, 15, or 16) ribonucleotides, and the second annealing region may include, e.g., from 6 to 50, e.g., from 10 to 16 (e.g., 10, 11, 12, 13, 14, 15, or 16) ribonucleotides.

In some embodiments, the 3′ half of Group I catalytic intron fragment and the 5′ half of Group I catalytic intron fragment are from a cyanobacterium Anabaena pre-tRNA-Leu gene, a Tetrahymena pre-rRNA, or a T4 phage td gene.

In some embodiments, the 3′ half of Group I catalytic intron fragment and the 5′ Group I catalytic intron fragment are from a T4 phage td gene. The 3′ exon fragment may include the first annealing region and the 5′ half of Group I catalytic intron fragment may include the second annealing region. The first annealing region may include, e.g., from 2 to 16, e.g., 10 to 16 (e.g., 2, 3, 4, 5, 6, 7, 8, 9, 10, 11, 12, 13, 14, 15, or 16) ribonucleotides, and the second annealing region may include, e.g., from 2 to 16, e.g., 10 to 16 (e.g., 2, 3, 4, 5, 6, 7, 8, 9, 10, 11, 12, 13, 14, 15, or 16) ribonucleotides.

In some embodiments, the 3′ half of Group I catalytic intron fragment is the 5′ terminus of the linear polynucleotide.

In some embodiments, the 5′ half of Group I catalytic intron fragment is the 3′ terminus of the linear polyribonucleotide.

In some embodiments, the 3′ half of Group I catalytic intron fragment has at least 80% (e.g., at least 85%, 90%, 95%, 97%, 99%, or 100%) sequence identity to the sequence of 5′-AACAACAGATAACTTACAGCTAGTCGGAAGGTGCAGAGACTCGACGGGAGCTACCCTAACGTCA AGACGAGGGTAAAGAGAGAGTCCAATTCTCAAAGCCAATAGGCAGTAGCGAAAGCTGCGGGAG AATG-3′ (SEQ ID NO: 215).

In some embodiments, the 5′ half of Group I catalytic intron fragment has at least 80% (e.g., at least 85%, 90%, 95%, 97%, 99%, or 100%) sequence identity to the sequence of 5′-AAATAATTGAGCCTTAGAGAAGAAATTCTTTAAGTGGATGCTCTCAAACTCAGGGAAACCTAAATC TAGCTATAGACAAGGCAATCCTGAGCCAAGCCGAAGTAGTAATTAGTAAGTT-3′ (SEQ ID NO: 216).

In some embodiments, the 3′ half of Group I catalytic intron fragment has the sequence of SEQ ID NO: 215 and the 5′ half of Group I catalytic intron fragment has the sequence of SEQ ID NO: 216.

In some embodiments, the 3′ half of Group I catalytic intron fragment has at least 80% (e.g., at least 85%, 90%, 95%, 97%, 99%, or 100%) sequence identity to the sequence of 5′-CTTCTGTTGATATGGATGCAGTTCACAGACTAAATGTCGGTCGGGGAAGATGTATTCTTCTCATA AGATATAGTCGGACCTCTCCTTAATGGGAGCTAGCGGATGAAGTGATGCAACACTGGAGCCGCT GGGAACTAATTTGTATGCGAAAGTATATTGATTAGTTTTGGAGTACTCG-3′ (SEQ ID NO: 217).

In some embodiments, the 5′ half of Group I catalytic intron fragment has at least 80% (e.g., at least 85%, 90%, 95%, 97%, 99%, or 100%) sequence identity to the sequence of 5′-AAATAGCAATATTTACCTTTGGAGGGAAAAGTTATCAGGCATGCACCTGGTAGCTAGTCTTTAAAC CAATAGATTGCATCGGTTTAAAAGGCAAGACCGTCAAATTGCGGGAAAGGGGTCAACAGCCGTT CAGTACCAAGTCTCAGGGGAAACTTTGAGATGGCCTTGCAAAGGGTATGGTAATAAGCTGACGG ACATGGTCCTAACCACGCAGCCAAGTCCTAAGTCAACAGAT-3′ (SEQ ID NO: 218).

In some embodiments, the 3′ half of Group I catalytic intron fragment has the sequence of SEQ ID NO: 217 and the 5′ half of Group I catalytic intron fragment has the sequence of SEQ ID NO: 218.

In some embodiments, the 3′ half of Group I catalytic intron fragment has at least 80% (e.g., at least 85%, 90%, 95%, 97%, 99%, or 100%) sequence identity to the sequence of 5′-GGTTCTACATAAATGCCTAACGACTATCCCTTTGGGGAGTAGGGTCAAGTGACTCGAAACGATAG ACAACTTGCTTTAACAAGTTGGAGATATAGTCTGCTCTGCATGGTGACATGCAGCTGGATATAATT CCGGGGTAAGATTAACGACCTTATCTGAACATAATG-3′ (SEQ ID NO: 219).

In some embodiments, the 5′ half of Group I catalytic intron fragment has at least 80% (e.g., at least 85%, 90%, 95%, 97%, 99%, or 100%) sequence identity to the sequence of 5′-TAATTGAGGCCTGAGTATAAGGTGACTTATACTTGTAATCTATCTAAACGGGGAACCTCTCTAGTA GACAATCCCGTGCTAAATTGTAGGACT-3′ (SEQ ID NO: 220).

In some embodiments, the 3′ half of Group I catalytic intron fragment has the sequence of SEQ ID NO: 219 and the 5′ half of Group I catalytic intron fragment has the sequence of SEQ ID NO: 220.

In some embodiments, the 3′ half of Group I catalytic intron fragment has at least 80% (e.g., at least 85%, 90%, 95%, 97%, 99%, or 100%) sequence identity to the sequence of 5′-TAAACAACTAACAGCTTTAGAAGGTGCAGAGACTAGACGGGAGCTACCCTAACGGATTCAGCCG AGGGTAAAGGGATAGTCCAATTCTCAACATCGCGATTGTTGATGGCAGCGAAAGTTGCAGAGAG AATGAAAATCCGCTGACTGTAAAGGTCGTGAGGGTTCGAGTCCCTCCGCCCCCA-3′ (SEQ ID NO: 221).

In some embodiments, the 5′ half of Group I catalytic intron fragment has at least 80% (e.g., at least 85%, 90%, 95%, 97%, 99%, or 100%) sequence identity to the sequence of 5′-ACGGTAGACGCAGCGGACTTAGAAAACTGGGCCTCGATCGCGAAAGGGATCGAGTGGCAGCTC TCAAACTCAGGGAAACCTAAAACTTTAAACATTMAAGTCATGGCAATCCTGAGCCAAGCTAAAGC-3′ (SEQ ID NO: 222).

In some embodiments, the 3′ half of Group I catalytic intron fragment has the sequence of SEQ ID NO: 221 and the 5′ half of Group I catalytic intron fragment has the sequence of SEQ ID NO: 222.

In some embodiments, the 3′ half of Group I catalytic intron fragment has at least 80% (e.g., at least 85%, 90%, 95%, 97%, 99%, or 100%) sequence identity to the sequence of 5′-TTAAACTCAAAATTTAAAATCCCAAATTCAAAATTCCGGGAAGGTGCAGAGACTCGACGGGAGCT ACCCTAACGTAAAGCCGAGGGTAAAGGGAGAGTCCAATTCTCAAAGCCTGAAGTTGCTGAAGCA ACAAGGCAGTAGTGAAAGCTGCGAGAGAATGAAAATCCGTTGACTGTAAAAAGTCGTGGGGGTT CAAGTCCCCCCACCCCC-3′ (SEQ ID NO: 223).

In some embodiments, the 5′ half of Group I catalytic intron fragment has at least 80% (e.g., at least 85%, 90%, 95%, 97%, 99%, or 100%) sequence identity to the sequence of 5′-ATGGTAGACGCTACGGACTTAGAAAACTGAGCCTTGATAGAGAAATCTTTTAAGTGGAAGCTCTC AAATTCAGGGAAACCTAAATCTGAATACAGATATGGCAATCCTGAGCCAAGCCCAGAAAATTTAG ACTTGAGATTTGATTTTGGAG-3′ (SEQ ID NO: 224).

In some embodiments, the 3′ half of Group I catalytic intron fragment has the sequence of SEQ ID NO: 223 and the 5′ half of Group I catalytic intron fragment has the sequence of SEQ ID NO: 224.

In some embodiments, the 3′ half of Group I catalytic intron fragment has at least 80% (e.g., at least 85%, 90%, 95%, 97%, 99%, or 100%) sequence identity to the sequence of 5′-GGCTTTCAATTTGAAATCAGAAATTCAAAATTCAGGGAAGGTGCAGAGACTCGACGGGAGCTACC CTAACGTAAAGGCGAGGGTAAAGGGAGAGTCCAATTCTTAAAGCCTGAAGTTGTGCAAGCAACA AGGCAACAGTGAAAGCTGTGGAAGAATGAAAATCCGTTGACCTTAAACGGTCGTGGGGGTTCAA GTCCCCCCACCCCC-3′ (SEQ ID NO: 225).

In some embodiments, the 5′ half of Group I catalytic intron fragment has at least 80% (e.g., at least 85%, 90%, 95%, 97%, 99%, or 100%) sequence identity to the sequence of 5′-ATGGTAGACGCTACGGACTTAGAAAACTGAGCCTTGATAGAGAAATCTTTCAAGTGGAAGCTCTC AAATTCAGGGAAACCTAAATCTGAATACAGATATGGCAATCCTGAGCCAAGCCCGGAAATTTTAG AATCAAGATTTTATTTT-3′ (SEQ ID NO: 226).

In some embodiments, the 3′ half of Group I catalytic intron fragment has the sequence of SEQ ID NO: 225 and the 5′ half of Group I catalytic intron fragment has the sequence of SEQ ID NO: 226.

In some embodiments, the 3′ half of Group I catalytic intron fragment has at least 80% (e.g., at least 85%, 90%, 95%, 97%, 99%, or 100%) sequence identity to the sequence of 5′-AGAAATGGAGAAGGTGTAGAGACTGGAAGGCAGGCACCCTAACGTTAAAGGCGAGGGTGAAGG GACAGTCCAGACCACAAACCAGTAAATCTGGGCAGCGAAAGCTGTAGATGGTAAGCATAACCCG AAGGTCAGTGGTTCAAATCCACTTCCCGCCACCAAATTAAAAAAACAATAA-3′ (SEQ ID NO: 227).

In some embodiments, the 5′ half of Group I catalytic intron fragment has at least 80% (e.g., at least 85%, 90%, 95%, 97%, 99%, or 100%) sequence identity to the sequence of 5′-AGAAATGGAGAAGGTGTAGAGACTGGAAGGCAGGCACCCTAACGTTAAAGGCGAGGGTGAAGG GACAGTCCAGACCACAAACCAGTAAATCTGGGCAGCGAAAGCTGTAGATGGTAAGCATAACCCG AAGGTCAGTGGTTCAAATCCACTTCCCGCCACCAAATTAAAAAAACAATAA-3′ (SEQ ID NO: 228).

In some embodiments, the 3′ half of Group I catalytic intron fragment has the sequence of SEQ ID NO: 227 and the 5′ half of Group I catalytic intron fragment has the sequence of SEQ ID NO: 228.

In some embodiments, the 3′ half of Group I catalytic intron fragment has at least 80% (e.g., at least 85%, 90%, 95%, 97%, 99%, or 100%) sequence identity to the sequence of 5′-ACAACAGATAACTTACTAACTTACAGCTAGTCGGAAGGTGCAGAGACTCGACGGGAGCTACCCT AACGTCAAGACGAGGGTAAAGAGAGAGTCCAATTCTCAAAGCCAATAGGCAGTAGCGAAAGCTG CGGGAGAATGAAAATCCGTAGCGTCTAAACGGTCGTGTGGGTTCAAGTCCCTCCACCCCCA-3′ (SEQ ID NO: 229).

In some embodiments, the 5′ half of Group I catalytic intron fragment has at least 80% (e.g., at least 85%, 90%, 95%, 97%, 99%, or 100%) sequence identity to the sequence of 5′-AGACGCTACGGACTTAAATAATTGAGCCTTAGAGAAGAAATTCTTTAAGTGGATGCTCTCAAACTC AGGGAAACCTAAATCTAGCTATAGACAAGGCAATCCTGAGCCAAGCCGAAGTAGTAATTAGTAAG TTAGTAAGTT-3′ (SEQ ID NO: 230).

In some embodiments, the 3′ half of Group I catalytic intron fragment has the sequence of SEQ ID NO: 229 and the 5′ half of Group I catalytic intron fragment has the sequence of SEQ ID NO: 230.

In some embodiments, the 3′ half of Group I catalytic intron fragment has at least 80% (e.g., at least 85%, 90%, 95%, 97%, 99%, or 100%) sequence identity to the sequence of 5′-AACAACAGATAACTTACTAGTTACTAGTCGGAAGGTGCAGAGACTCGACGGGAGCTACCCTAAC GTCAAGACGAGGGTAAAGAGAGAGTCCAATTCTCAAAGCCAATAGGCAGTAGCGAAAGCTGCGG GAGAATGAAAATCCGTAGCGTCTAAACGGTCGTGTGGGTTCAAGTCCCTCCACCCCCA-3′ (SEQ ID NO: 231).

In some embodiments, the 5′ half of Group I catalytic intron fragment has at least 80% (e.g., at least 85%, 90%, 95%, 97%, 99%, or 100%) sequence identity to the sequence of 5′ -AGACGCTACGGACTTAAATAATTGAGCCTTAGAGAAGAAATTCTTTAAGTGGATGCTCTCAAACTC AGGGAAACCTAAATCTAGCTATAGACAAGGCAATCCTGAGCCAAGCCGAAGTAGTAATTAGTAAG TT-3′ (SEQ ID NO: 232).

In some embodiments, the 3′ half of Group I catalytic intron fragment has the sequence of SEQ ID NO: 231 and the 5′ half of Group I catalytic intron fragment has the sequence of SEQ ID NO: 232.

In some embodiments, the Group I catalytic intron fragment is from the T4 phage nrdB gene or nrdD gene. In some embodiments, the 3′ half of Group I catalytic intron fragment of includes a sequence having at least 80% sequence identity to 5′-TTGCAAAACAAGGTTCAACGACTAGTCTTCGGACGTAGGGTCAAGCGACTCGAAATGGGGAGAA TCCCTCCGGGATTGTGATATAGTCTGGACTGCATGGTAACATGCAGCAGTTCATAAGAGAACGG GTTGAGAATTAGCGAGCTCAATCGAACATACG-3′ (SEQ ID NO: 233).

In some embodiments, the 3′ half of Group I catalytic intron fragment of (A) includes a sequence having at least 85%, 90%, 95%, 97%, 99%, or 100% sequence identity to 5′-TTGCAAAACAAGGTTCAACGACTAGTCTTCGGACGTAGGGTCAAGCGACTCGAAATGGGGAGAA TCCCTCCGGGATTGTGATATAGTCTGGACTGCATGGTAACATGCAGCAGTTCATAAGAGAACGG GTTGAGAATTAGCGAGCTCAATCGAACATACG-3′ (SEQ ID NO: 233).

In some embodiments, the 5′ half of Group I catalytic intron fragment from the T4 phage nrdB gene. In some embodiments, the 3′ half of Group I catalytic intron fragment is from the T4 phage nrdB gene and the 5′ half of Group I catalytic intron fragment is from the T4 phage nrdB gene.

In some embodiments, the 5′ half of Group I catalytic intron includes a sequence having at least 80% sequence identity to 5′-AAAATGCGCCTTTAAACGGTAACGTTTATCGAAAACTCCTTTAATTGCTGGAAAGTCCTTTATGGA AAACTAGCAGCCAAGGTTTTGCTT-3′ (SEQ ID NO: 235).

In some embodiments, the 5′ half of Group I catalytic intron includes a sequence having at least 85%, 90%, 95%, 97%, 99%, or 100% sequence identity to 5′-AAAATGCGCCTTTAAACGGTAACGTTTATCGAAAACTCCTTTAATTGCTGGAAAGTCCTTTATGGA AAACTAGCAGCCAAGGTTTTGCTT-3′ (SEQ ID NO: 235).

In some embodiments, the 3′ half of Group I catalytic intron fragment is from the T4 phage nrdD gene.

In some embodiments, the 3′ half of Group I catalytic intron fragment includes a sequence having at least 80% sequence identity to 5′-CAGTAGCTGTAAATGCCCAACGACTATCCCTGATGAATGTAAGGGAGTAGGGTCAAGCGACCCG AAACGGCAGACAACTCTAAGAGTTGAAGATATAGTCTGAACTGCATGGTGACATGCAGCTGTTTA TCCTCGTATAAATATGAATACGAGGTGAAACGATGAAATGAATTACATTGTTTCATATAAACGGGT AGAGAAGTAGCGAACTCTACTGAACACATTG-3′ (SEQ ID NO: 237).

In some embodiments, the 3′ half of Group I catalytic intron fragment includes a sequence having at least 85%, 90%, 95%, 97%, 99%, or 100% sequence identity to 5′-CAGTAGCTGTAAATGCCCAACGACTATCCCTGATGAATGTAAGGGAGTAGGGTCAAGCGACCCG AAACGGCAGACAACTCTAAGAGTTGAAGATATAGTCTGAACTGCATGGTGACATGCAGCTGTTTA TCCTCGTATAAATATGAATACGAGGTGAAACGATGAAATGAATTACATTGTTTCATATAAACGGGT AGAGAAGTAGCGAACTCTACTGAACACATTG-3′ (SEQ ID NO: 237).

In some embodiments, the 5′ half of Group I catalytic intron fragment is from the T4 phage nrdD gene. In some embodiments, the 3′ half of Group I catalytic intron fragment is from the T4 phage nrdD gene and the 5′ half of Group I catalytic intron fragment is from the T4 phage nrdD gene.

In some embodiments, the 5′ half of Group I catalytic intron includes a sequence having at least 80% sequence identity to 5′-TAACGTAAGTCAAGCTCATGTAAAATCTGCCTAAAACGGGAAACTCTCACTGAGACAATCCGTTG CTAAATCAG-3′ (SEQ ID NO: 239).

In some embodiments, the 5′ half of Group I catalytic intron includes a sequence having at least 85%, 90%, 95%, 97%, 99%, or 100% sequence identity to 5′-TAACGTAAGTCAAGCTCATGTAAAATCTGCCTAAAACGGGAAACTCTCACTGAGACAATCCGTTG CTAAATCAG-3′ (SEQ ID NO: 239).

In some embodiments, the 3′ exon fragment includes a sequence having at least 80% sequence identity to 5′-GTACCTTTAACTTCCATAAGAACATGGAAATCATGGAAGGTAATGCCAAG-3′ (SEQ ID NO: 241).

In some embodiments, the 3′ exon fragment includes a sequence having at least 85%, 90%, 95%, 97%, 99%, or 100% sequence identity to 5′-GTACCTTTAACTTCCATAAGAACATGGAAATCATGGAAGGTAATGCCAAG-3′ (SEQ ID NO: 241).

In some embodiments, the 3′ exon fragment includes a sequence having at least 80% sequence identity to 5′-GTACCTTTAACTTCCAAAAGATACATAAAAATCATGGAAGGTAATGCCAAG-3′ (SEQ ID NO: 243.

In some embodiments, the 3′ exon fragment includes a sequence having at least 85%, 90%, 95%, 97%, 99%, or 100% sequence identity to 5′-GTACCTTTAACTTCCAAAAGATACATAAAAATCATGGAAGGTAATGCCAAG-3′ (SEQ ID NO: 243).

In some embodiments, the 5′ exon fragment includes a sequence having at least 80% sequence identity to 5′-TTTTTATGTATOTTTTGCGT-3′ (SEQ ID NO: 245).

In some embodiments, the 5′ exon fragment includes a sequence having at least 85%, 90%, 95%, 97%, 99%, or 100% sequence identity to 5′-TTTTTATGTATOTTTTGCGT-3′ (SEQ ID NO: 245).

In some embodiments, the 3′ exon fragment Includes a sequence having at least 80% sequence identity to 5′-ATGAAGTGAACACGTTATTCAGTTCAAACGGACAGACTCCTTTTGTAACA-3′ (SEQ ID NO: 247).

In some embodiments, the 3′ exon fragment includes a sequence having at least 85%, 90%, 95%, 97%, 99%, or 100% sequence identity to 5′-ATGAAGTGAACACGTTATTCAGTTCAAACGGACAGACTCCTTTTGTAACA-3′ (SEQ ID NO: 247).

In some embodiments, the 3′ exon fragment includes a sequence having at least 80% sequence identity to 5′-ATGAAGTGAACACGTTACATAAGCTTGGAATGCAGACTCCTTTTGTAACA-3′ (SEQ ID NO: 249).

In some embodiments, the 3′ exon fragment includes a sequence having at least 85%, 90%, 95%, 97%, 99%, or 100% sequence identity to 5′-ATGAAGTGAACACGTTACATAAGCTTGGAATGCAGACTCCTTTTGTAACA-3′ (SEQ ID NO: 249).

In some embodiments, the 5′ exon fragment includes a sequence having at least 80% sequence identity to 5′-TGCATTCCAAGCTTATGAGT-3′ (SEQ ID NO: 251).

In some embodiments, the 5′ exon fragment includes a sequence having at least 85%, 90%, 95%, 97%, 99%, or 100% sequence identity to 5′-TGCATTCCAAGCTTATGAGT-3′ (SEQ ID NO: 251).

In another aspect, a linear polyribonucleotide for circularization may be cyclized or concatenated by a non-nucleic acid moiety that causes an attraction between atoms, molecular surfaces at, near, or linked to the 5′ and 3′ ends of the linear polyribonucleotide for circularization. In one embodiment, one or more linear polyribonucleotides is cyclized or concatenated by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In some embodiments, a linear polyribonucleotide for circularization may include a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3 ‘terminus may associate with each other causing a linear polyribonucleotide to cyclize or concatenate. In another example, the peptides covalently linked to the ribozyme RNA near the 5’ terminus and the 3′ terminus may cause the linear primary construct or linear mRNA to cyclize or concatenate after being subjected to ligation using various methods known in the art such as, but not limited to, protein ligation. Non-limiting examples of ribozymes for use in the linear primary constructs or linear polyribonucleotides of the present invention or a non-exhaustive listing of methods to incorporate or covalently link peptides are described in US patent application No. US20030082768, the contents of which is here in incorporated by reference in its entirety.

In another aspect, chemical methods of circularization may be used to generate the circular polyribonucleotide. Such methods may include but are not limited to click chemistry (e.g., alkyne and azide-based methods, or clickable bases), olefin metathesis, phosphoramidate ligation, hemiaminal-imine crosslinking, base modification, and any combination thereof. In some chemical methods, the 5′-end and the 3′-end of a linear polyribonucleotide for circularization includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In another aspect, the circular polyribonucleotide may be produced using a deoxyribonucleotide template transcribed in a cell-free system (e.g., by in vitro transcription) to a produce a linear RNA. The linear polyribonucleotide produces a splicing-compatible polyribonucleotide, which may be self-spliced to produce a circular polyribonucleotide.

In some embodiments, the disclosure provides a method of producing a circular polyribonucleotide (e.g., in a cell-free system) by providing a linear polyribonucleotide; and self-splicing linear polyribonucleotide under conditions suitable for splicing of the 3′ and 5′ splice sites of the linear polyribonucleotide; thereby producing a circular polyribonucleotide.

In some embodiments, the disclosure provides a method of producing a circular polyribonucleotide by providing a deoxyribonucleotide encoding the linear polyribonucleotide; transcribing the deoxyribonucleotide in a cell-free system to produce the linear polyribonucleotide; optionally purifying the splicing-compatible linear polyribonucleotide; and self-splicing the linear polyribonucleotide under conditions suitable for splicing of the 3′ and 5′ splice sites of the linear polyribonucleotide, thereby producing a circular polyribonucleotide.

In some embodiments, the disclosure provides a method of producing a circular polyribonucleotide by providing a deoxyribonucleotide encoding a linear polyribonucleotide; transcribing the deoxyribonucleotide in a cell-free system to produce the linear polyribonucleotide, wherein the transcribing occurs in a solution under conditions suitable for splicing of the 3′ and 5′ splice sites of the linear polyribonucleotide, thereby producing a circular polyribonucleotide. In some embodiments, the linear polyribonucleotide comprises a 5′ split-intron and a 3′ split-intron (e.g., a self-splicing construct for producing a circular polyribonucleotide). In some embodiments, the linear polyribonucleotide comprises a 5′ annealing region and a 3′ annealing region.

Suitable conditions for in vitro transcriptions and or self-splicing may include any conditions (e.g., a solution or a buffer, such as an aqueous buffer or solution) that mimic physiological conditions in one or more respects. In some embodiments, suitable conditions include between 0.1-100 mM Mg2+ ions or a salt thereof (e.g., 1-100 mM, 1-50 mM, 1-20 mM, 5-50 mM, 5-20 mM, or 5-15 mM). In some embodiments, suitable conditions include between 1-1000 mM K+ ions or a salt thereof such as KCl (e.g., 1-1000 mM, 1-500 mM, 1-200 mM, 50-500 mM, 100-500 mM, or 100-300 mM). In some embodiments, suitable conditions include between 1-1000 mM Cl— ions or a salt thereof such as KCl (e.g., 1-1000 mM, 1-500 mM, 1-200 mM, 50-500 mM, 100-500 mM, or 100-300 mM). In some embodiments, suitable conditions include between 0.1-100 mM Mn2+ ions or a salt thereof such as MnCl2 (e.g., 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM). In some embodiments, suitable conditions include dithiothreitol (DTT) (e.g., 1-1000 μM, 1-500 μM, 1-200 μM, 50-500 μM, 100-500 μM, 100-300 μM, 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM). In some embodiments, suitable conditions include between 0.1 mM and 100 mM ribonucleoside triphosphate (NTP) (e.g., 0.1-100 mM, 0.1-50 mM, 0.1-10 mM, 1-100 mM, 1-50 mM, or 1-10 mM). In some embodiments, suitable conditions include a pH of 4 to 10 (e.g., pH of 5 to 9, pH of 6 to 9, or pH of 6.5 to 8.5). In some embodiments, suitable conditions include a temperature of 4° C. to 50° C. (e.g., 10° C. to 40° C., 15° C. to 40° C., 20° C. to 40° C., or 30° C. to 40° C.),

In some embodiments the linear polyribonucleotide is produced from a deoxyribonucleic acid, e.g., a deoxyribonucleic acid described herein, such as a DNA vector, a linearized DNA vector, or a cDNA. In some embodiments, the linear polyribonucleotide is transcribed from the deoxyribonucleic acid by transcription in a cell-free system (e.g., in vitro transcription).

In another aspect, the circular polyribonucleotide may be produced in a cell, e.g., a prokaryotic cell or a eukaryotic cell. In some embodiments, an exogenous polyribonucleotide is provided to a cell (e.g., a linear polyribonucleotide described herein or a DNA molecule encoding for the transcription of a linear polyribonucleotide described here). The linear polyribonucleotides may be transcribed in the cell from an exogenous DNA molecule provided to the cell. The linear polyribonucleotide may be transcribed in the cell from an exogenous recombinant DNA molecule transiently provided to the cell. In some embodiments, the exogenous DNA molecule does not integrate into the cell's genome. In some embodiments, the linear polyribonucleotide is transcribed in the cell from a recombinant DNA molecule that is incorporated into the cell's genome.

In some embodiments, the cell is a prokaryotic cell. In some embodiments, the prokaryotic cell including the polyribonucleotides described herein is a bacterial cell or an archaeal cell. For example, the prokaryotic cell including the polyribonucleotides described herein may be E coli, halophilic archaea (e.g., Haloferax volcanii), Sphingomonas, cyanobacteria (e.g., Synechococcus elongatus, Spirulina (Arthrospira) spp., and Synechocystis spp.), Streptomyces, actinomycetes (e.g., Nonomuraea, Kitasatospora, or Thermobifida), Bacillus spp. (e.g., Bacillus subtilis, Bacillus anthracis, Bacillus cereus), betaproteobacteria (e.g., Burkholderia), alphaproteobacterial (e.g., Agrobacterium), Pseudomonas (e.g., Pseudomonas putida), and enterobacteria. The prokaryotic cells may be grown in a culture medium. The prokaryotic cells may be contained in a bioreactor.

In some embodiments, the cell is a eukaryotic cell. In some embodiments, the eukaryotic cell is a unicellular eukaryotic cell. In some embodiments, the unicellular eukaryotic is a unicellular fungal cell such as a yeast cell (e.g., Saccharomyces cerevisiae and other Saccharomyces spp., Brettanomyces spp., Schizosaccharomyces spp., Torulaspora spp, and Pichia spp.). In some embodiments, the unicellular eukaryotic cell is a unicellular animal cell. A unicellular animal cell may be a cell isolated from a multicellular animal and grown in culture, or the daughter cells thereof. In some embodiments, the unicellular animal cell is dedifferentiated. In some embodiments, the unicellular eukaryotic cell is a unicellular plant cell. A unicellular plant cell may be a cell isolated from a multicellular plant and grown in culture, or the daughter cells thereof. In some embodiments, the unicellular plant cell is dedifferentiated. In some embodiments, the unicellular plant cell is from a plant callus. In embodiments, the unicellular cell is a plant cell protoplast. In some embodiments, the unicellular eukaryotic cell is a unicellular eukaryotic algal cell, such as a unicellular green alga, a diatom, an euglenoid, or a dinoflagellate. Non-limiting examples of unicellular eukaryotic algae of interest include Dunaliella salina, Chlorella vulgaris, Chlorella zofingiensis, Haematococcus pluvialis, Neochloris oleoabundans and other Neochloris spp., Protosiphon botryoides, Botryococcus braunii, Cryptococcus spp., Chlamydomonas reinhardtii and other Chlamydomonas spp. In some embodiments, the unicellular eukaryotic cell is a protist cell. In some embodiments, the unicellular eukaryotic cell is a protozoan cell.

In some embodiments, the eukaryotic cell is a cell of a multicellular eukaryote. For example, the multicellular eukaryote may be selected from the group consisting of a vertebrate animal, an invertebrate animal, a multicellular fungus, a multicellular alga, and a multicellular plant. In some embodiments, the eukaryotic organism is a human. In some embodiments, the eukaryotic organism is a non-human vertebrate animal. In some embodiments, the eukaryotic organism is an invertebrate animal. In some embodiments, the eukaryotic organism is a multicellular fungus. In some embodiments, the eukaryotic organism is a multicellular plant. In embodiments, the eukaryotic cell is a cell of a human or a cell of a non-human mammal such as a non-human primate (e.g., monkeys, apes), ungulate (e.g., bovids including cattle, buffalo, bison, sheep, goat, and musk ox; pig; camelids including camel, llama, and alpaca; deer, antelope; and equids including horse and donkey), carnivore (e.g., dog, cat), rodent (e.g., rat, mouse, guinea pig, hamster, squirrel), or lagomorph (e.g., rabbit, hare). In embodiments, the eukaryotic cell is a cell of a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the eukaryotic cell is a cell of an arthropod (e.g., insects, arachnids, crustaceans), a nematode, an annelid, a helminth, or a mollusc. In embodiments, the eukaryotic cell is a cell of a multicellular plant, such as an angiosperm plant (which can be a dicot or a monocot) or a gymnosperm plant (e.g., a conifer, a cycad, a geophyte, a Ginkgo), a fern, horsetail, clubmoss, or a bryophyte. In embodiments, the eukaryotic cell is a cell of a eukaryotic multicellular alga.

The eukaryotic cells may be grown in a culture medium. The eukaryotic cells may be contained in a bioreactor.

In some embodiments, any method of producing a circular polyribonucleotide described herein may be performed in a bioreactor. A bioreactor refers to any vessel in which a chemical or biological process is carried out which involves organisms or biochemically active substances derived from such organisms. Bioreactors may be compatible with the cell-free methods for production of circular RNA described herein. A vessel for a bioreactor may include a culture flask, a dish, or a bag that may be single use (disposable), autoclavable, or sterilizable. A bioreactor may be made of glass, or it may be polymer-based, or it may be made of other materials.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor may be a batch or continuous processes. A bioreactor is continuous when the reagent and product streams are continuously being fed and withdrawn from the system. A batch bioreactor may have a continuous recirculating flow, but no continuous feeding of reagents or product harvest. Some methods of the present disclosure are directed to large-scale production of circular polyribonucleotides. For large-scale production methods, the method may be performed in a volume of 1 liter (L) to 50 L, or more (e.g., 5 L, 10 L, 15 L, 20 L, 25 L, 30 L, 35 L, 40 L, 45 L, 50 L, or more). In some embodiments, the method may be performed in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L. In some embodiments, a bioreactor may produce at least 1 g of circular RNA. In some embodiments, a bioreactor may produce 1-200 g of circular RNA (e.g., 1-10 g, 1-20 g, 1-50 g, 10-50 g, 10-100 g, 50-100 g, or 50-200 g of circular RNA). In some embodiments, the amount produced is measured per liter (e.g., 1-200 g per liter), per batch or reaction (e.g., 1-200 g per batch or reaction), or per unit time (e.g., 1-200 g per hour or per day). In some embodiments, more than one bioreactor may be utilized in series to increase the production capacity (e.g., one, two, three, four, five, six, seven, eight, or nine bioreactors may be used in series).

In some embodiments, circularization efficiency is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, circularization efficiency is at least about 40%. In some embodiments, circularization efficiency is between about 10% and about 100%; for example, circularization efficiency is about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In some embodiments, circularization efficiency is between about 20% and about 80%. In some embodiments, circularization efficiency is between about 30% and about 60%. In some embodiments, circularization efficiency is about 40%.

Additional methods of making the circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); Muller and Appel, from RNA Biol, 2017, 14(8):1018-1027; and Egli & Herdewijn, Chemistry and Biology of Artificial Nucleic Acids, (First Edition), Wiley-VCH (2012). Other methods of making circular polyribonucleotides are described, for example, in International Publication No. WO2022/247943, US patent No. U.S. Pat. No. 11,000,547, International Publication No. 2018/191722, International Publication No. WO2019/236673, International Publication No. WO2020/023595, International Publication No. WO2022/204460, International Publication No. WO2022/204464, International Publication No. WO2022/204466, and International Publication No. 2022/261490.

Additional methods of synthesizing circular polyribonucleotides are also described elsewhere (see, e.g., US patent No. U.S. Pat. No. 6,210,931, US Patent No. U.S. Pat. No. 5,773,244, US Patent No. U.S. Pat. No. 5,766,903, US Patent No. U.S. Pat. No. 5,712,128, US patent No. U.S. Pat. No. 5,426,180, US Publication No. US20100137407, International Publication No. WO1992001813, International Publication No. WO2010084371, and Petkovic et al., Nucleic Acids Res. 43:2454-65 (2015); the contents of each of which are herein incorporated by reference in their entirety).

### Methods of Purification

One or more purification steps may be included in the methods described herein. For example, in some embodiments, the linear polyribonucleotide is substantively enriched or pure (e.g., purified) prior to self-splicing the linear polyribonucleotide. In other embodiments, the linear polyribonucleotide is not purified prior to self-splicing the linear polyribonucleotide. In some embodiments, the resulting circular RNA is purified.

Purification may include separating or enriching the desired reaction product from one or more undesired components, such as any unreacted stating material, byproducts, enzymes, or other reaction components. For example, purification of linear polyribonucleotide following transcription in a cell-free system (e.g., in vitro transcription) may include separation or enrichment from the DNA template prior to self-splicing the linear polyribonucleotide. Purification of the circular RNA product following splicing may be used to separate or enrich the circular polyribonucleotide from its corresponding linear polyribonucleotide. Methods of purification of polyribonucleotides are known to those of skill in the art and include enzymatic purification or by chromatography.

In some embodiments, the methods of purification result in a circular polyribonucleotide that has less than 50% (e.g., less than 40%, 30%, 20%, 10%, 5%, 4%, 3%, 2%, or 1%) linear polyribonucleotides.

In some embodiments, the reference criterion for the amount of linear polyribonucleotide molecules present in a preparation (e.g., pharmaceutical or agricultural preparation) is the presence of no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules.

In some embodiments, the reference criterion for the amount of circular polyribonucleotide molecules present in a preparation (e.g., pharmaceutical or agricultural preparation) is at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), 99.9% (w/w), or 100% (w/w) molecules of the total ribonucleotide molecules in the preparation.

In some embodiments, the reference criterion for the amount of linear polyribonucleotide molecules present in a preparation (e.g., pharmaceutical or agricultural preparation) is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) linear polyribonucleotide molecules of the total ribonucleotide molecules in the preparation.

In some embodiments, the reference criterion for the amount of nicked polyribonucleotide molecules present in a preparation (e.g., pharmaceutical or agricultural preparation) is no more than 0.5% (w/w), 1% (w/w), 2% (w/w), 5% (w/w), 10% (w/w), or 15% (w/w) nicked polyribonucleotide molecules of the total ribonucleotide molecules in the preparation.

In some embodiments, the reference criterion for the amount of combined nicked and linear polyribonucleotide molecules present in a preparation (e.g., pharmaceutical or agricultural preparation) is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) combined nicked and linear polyribonucleotide molecules of the total ribonucleotide molecules in the preparation. In some embodiments, a preparation (e.g., pharmaceutical or agricultural preparation) is an intermediate preparation of a final circular polyribonucleotide drug product. In some embodiments, a preparation (e.g., pharmaceutical or agricultural preparation) is a drug substance or active pharmaceutical ingredient (API). In some embodiments, a preparation (e.g., pharmaceutical or agricultural preparation) is a drug product for administration to a subject.

In some embodiments, a preparation (e.g., pharmaceutical or agricultural preparation) of circular polyribonucleotides is (before, during or after the reduction of linear polyribonucleotide) further processed to substantially remove DNA, protein contamination (e.g., cell protein such as a host cell protein or protein process impurities), endotoxin, mononucleotide molecules, and/or a process-related impurity.

### Linear Polyribonucleotide Elements

The linear polyribonucleotide comprises the elements as described below as described herein.

Linear polyribonucleotides described herein are a polyribonucleotide molecule having a 5′ and 3′ end. In some embodiments, the linear RNA has a free 5′ end or 3′ end. In some embodiments, the linear RNA has a 5′ end or 3′ end that is modified or protected from degradation. In some embodiments, the linear RNA has non-covalently linked 5′ or 3′ ends. In some embodiments, the linear RNA is an mRNA.

In some embodiments, the linear polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

The linear polyribonucleotides of the disclosure may include any element or combination of elements described herein, e.g., any element or combination of elements described above with respect to circular polyribonucleotides. A linear polyribonucleotide may include any one or more IRES, signal sequence, regulatory element, cleavage domain, translation initiation sequence, untranslated region, termination element, or modification as described herein (e.g., with respect to circular polyribonucleotide described above). A linear polyribonucleotide may include such elements in any number or configuration described herein (e.g., with respect to circular polyribonucleotide described above).

### Methods of Use

A polyribonucleotide described herein may be administered to a subject (e.g., in a pharmaceutical or agricultural composition). For example, a circular or linear polyribonucleotide described herein may be administered to a subject (e.g., in a pharmaceutical or agricultural composition). In some embodiments, a circular or linear polyribonucleotide described herein is delivered to a cell.

In some embodiments, a polyribonucleotide (e.g., a circular polyribonucleotide, a linear polyribonucleotide) described herein (e.g., in a pharmaceutical or agricultural composition) is used for the treatment, amelioration, and/or prevention of a syndrome, condition, disease, and/or disorder.

### Methods of Dosing

A polyribonucleotide (e.g., a circular polyribonucleotide, a linear polyribonucleotide) described herein (e.g., in a pharmaceutical or agricultural composition) may be administered in a single dose or in multiple doses to a cell, tissue, or subject.

A method of administering multiple doses of a composition of a polyribonucleotide described herein (e.g., a circular polyribonucleotide, a linear polyribonucleotide) (e.g., in a pharmaceutical or agricultural composition) comprises providing two or more compositions, over a period of time, to a cell, tissue or subject (e.g., a mammal). According to certain embodiments, multiple doses of a composition of a polyribonucleotide described herein (e.g., a circular polyribonucleotide, a linear polyribonucleotide) (e.g., in a pharmaceutical or agricultural composition) may be administered to a subject over a defined time course. The methods according to this aspect of the invention comprise sequentially administering to a subject multiple doses of a composition of a polyribonucleotide described herein (e.g., a circular polyribonucleotide, a linear polyribonucleotide) (e.g., in a pharmaceutical or agricultural composition). As used herein, “sequentially administering” means that each dose of composition of a polyribonucleotide described herein (e.g., a circular polyribonucleotide, a linear polyribonucleotide) (e.g., in a pharmaceutical or agricultural composition) is administered to the subject at a different point in time, e.g., on different days separated by a predetermined interval (e.g., hours, days, weeks or months). In some embodiments, the present invention provides methods which comprise sequentially administering to the subject a single initial dose of a composition of a polyribonucleotide described herein (e.g., a circular polyribonucleotide, a linear polyribonucleotide) (e.g., in a pharmaceutical or agricultural composition), followed by one or more secondary doses of the composition, and optionally followed by one or more tertiary doses of the composition.

The terms “initial dose,” “secondary doses,” and “tertiary doses,” refer to the temporal sequence of administration of a composition of a polyribonucleotide described herein (e.g., a circular polyribonucleotide, a linear polyribonucleotide) (e.g., in a pharmaceutical or agricultural composition). Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of a composition of a polyribonucleotide described herein (e.g., a circular polyribonucleotide, a linear polyribonucleotide) (e.g., in a pharmaceutical or agricultural composition), and in certain embodiments, may differ from one another in terms of frequency of administration. In certain embodiments, the amount of a composition of a polyribonucleotide described herein (e.g., a circular polyribonucleotide, a linear polyribonucleotide) (e.g., in a pharmaceutical or agricultural composition) contained in the initial, secondary and/or tertiary doses varies from one another (e.g., adjusted up or down as appropriate) during the course of treatment. In certain embodiments, one or more (e.g., 2, 3, 4, or 5) doses are administered at the beginning of the treatment regimen as “loading doses” followed by subsequent doses that are administered on a less frequent basis (e.g., “maintenance doses”).

In certain embodiments, each secondary and/or tertiary dose is administered after the immediately preceding dose. The phrase “the immediately preceding dose,” as used herein, means, in a sequence of multiple administrations, the dose of the composition of a polyribonucleotide described herein (e.g., a circular polyribonucleotide, a linear polyribonucleotide) (e.g., in a pharmaceutical or agricultural composition) which is administered to a subject prior to the administration of the very next dose in the sequence with no intervening doses. In certain embodiments, each secondary and/or tertiary dose is administered every day, every 2 days, 3 days, 4 days, 5 days, 6 days, or 7 days after the immediately preceding dose. In certain embodiments, each secondary and/or tertiary dose is administered every 0.5 weeks, 1 week, 2 weeks, 3 weeks, or 4 weeks after the immediately preceding dose.

The methods according to this aspect of the invention may comprise administering to a subject any number of secondary and/or tertiary doses of a composition of a polyribonucleotide described herein (e.g., a circular polyribonucleotide, a linear polyribonucleotide) (e.g., in a pharmaceutical or agricultural composition). For example, in certain embodiments, only a single secondary dose is administered to the subject. In other embodiments, two or more (e.g., 2, 3, 4, 5, 6, 7, 8, or more) secondary doses are administered to the subject. Likewise, in certain embodiments, only a single tertiary dose is administered to the subject. In other embodiments, two or more (e.g., 2, 3, 4, 5, 6, 7, 8, or more) tertiary doses are administered to the subject.

In certain embodiments, the frequency at which the secondary and/or tertiary doses are administered to a subject can vary over the course of the treatment regimen. The frequency of administration may also be adjusted during the course of treatment.

In some embodiments, multiple doses are provided to produce a level of the composition or express a level of the encoded polypeptide in a cell, tissue or subject. In some embodiments, multiple doses are provided to produce or maintain a level of the composition, or to produce or maintain a level of the encoded polypeptide, in a cell, tissue or subject for a period of time, for instance, for at least 20, 30, 40, 50, 60, 70, 80, 90, 100, 120, 150 days, or at least 1, 2, 3, 4, 5, 6, 7, 8, 9, 10, 12, 15, 18, 21, or 24 months, or at least 1, 2, 3, 4, or 5 years.

In some embodiments, the method comprises providing (e.g., administering) at least a first composition and a second composition to the cells, tissue, or subject (e.g., a mammal, e.g., a human). In some embodiments, the method further comprises providing (e.g., administering) a third composition, fourth composition, fifth composition, sixth composition, seventh composition, eighth composition, ninth composition, tenth composition, or more. In some embodiments, additional compositions are provided for the duration of the life of the cell. In some embodiments, additional compositions are provided (e.g., administered) while the cell, tissue or subject obtains a benefit from the composition.

In some embodiments, a first composition in a multiple dosing regimen comprises a first amount of the polyribonucleotide (e.g., circular polyribonucleotide, linear polyribonucleotide) disclosed herein. In some embodiments, a second composition in a multiple dosing regimen comprises a second amount of the polyribonucleotide (e.g., circular polyribonucleotide, linear polyribonucleotide) disclosed herein. In some embodiments, a third composition, a fourth composition, a fifth composition, a sixth composition, a seventh composition, an eighth composition, a ninth composition, a tenth composition, or more in a multiple dosing regimen comprises a third, fourth, fifth, sixth, seventh, eighth, ninth, tenth or more amount of the polyribonucleotide (e.g., circular polyribonucleotide, linear polyribonucleotide) disclosed herein. In some embodiments, the second amount of the polyribonucleotide (e.g., circular polyribonucleotide, linear polyribonucleotide) is the same as the first amount of the polyribonucleotide (e.g., circular polyribonucleotide, linear polyribonucleotide). In some embodiments, the third amount of the polyribonucleotide (e.g., circular polyribonucleotide, linear polyribonucleotide) is the same as the first amount of the polyribonucleotide (e.g., circular polyribonucleotide, linear polyribonucleotide). In some embodiments, the fourth, fifth, sixth, seventh, eighth, ninth, tenth, or more amount of the polyribonucleotide (e.g., circular polyribonucleotide, linear polyribonucleotide) is the same as the first amount of the polyribonucleotide (e.g., circular polyribonucleotide, linear polyribonucleotide). In some embodiments, the second amount of the polyribonucleotide (e.g., circular polyribonucleotide, linear polyribonucleotide) is less than the first amount of the polyribonucleotide (e.g., circular polyribonucleotide, linear polyribonucleotide). In some embodiments, the third amount of the polyribonucleotide (e.g., circular polyribonucleotide, linear polyribonucleotide) is less than the first amount of the polyribonucleotide (e.g., circular polyribonucleotide, linear polyribonucleotide). In some embodiments, the fourth, fifth, sixth, seventh, eighth, ninth, tenth, or more amount of the polyribonucleotide (e.g., circular polyribonucleotide, linear polyribonucleotide) is less than the first amount of the polyribonucleotide (e.g., circular polyribonucleotide, linear polyribonucleotide). In some embodiments, the second amount of the polyribonucleotide (e.g., circular polyribonucleotide, linear polyribonucleotide) is greater than the first amount of the polyribonucleotide (e.g., circular polyribonucleotide, linear polyribonucleotide). In some embodiments, the third amount of the polyribonucleotide (e.g., circular polyribonucleotide, linear polyribonucleotide) is greater than the first amount of the polyribonucleotide (e.g., circular polyribonucleotide, linear polyribonucleotide). In some embodiments, the fourth, fifth, sixth, seventh, eighth, ninth, tenth, or more amount of the polyribonucleotide (e.g., circular polyribonucleotide, linear polyribonucleotide) is greater than the first amount of the polyribonucleotide (e.g., circular polyribonucleotide, linear polyribonucleotide). In some embodiments, an amount of the polyribonucleotide (e.g., circular polyribonucleotide, linear polyribonucleotide) of the second composition varies by no more than 1%, 5%, 10%, 15%, 20%, or 25% of an amount of the polyribonucleotide (e.g., circular polyribonucleotide, linear polyribonucleotide) of the first composition. In some embodiments, an amount of the polyribonucleotide (e.g., circular polyribonucleotide, linear polyribonucleotide) of the second composition is no more than 1%, 5%, 10%, 15%, 20%, or 25% less than an amount of the polyribonucleotide (e.g., circular polyribonucleotide, linear polyribonucleotide) of the first composition. In some embodiments, an amount of the polyribonucleotide (e.g., circular polyribonucleotide, linear polyribonucleotide) of a second composition is from 0.1-fold to 1000-fold higher than an amount of the polyribonucleotide (e.g., circular polyribonucleotide, linear polyribonucleotide) of a first composition. In some embodiments, an amount of the polyribonucleotide (e.g., circular polyribonucleotide, linear polyribonucleotide) of a second composition is 0.1-fold, 1-fold, 5-fold, 10-fold, 100-fold, or 1000-fold higher than an amount of the polyribonucleotide (e.g., circular polyribonucleotide, linear polyribonucleotide) of a first composition. In some embodiments, an amount of the polyribonucleotide (e.g., circular polyribonucleotide, linear polyribonucleotide) of a subsequent composition (e.g., a composition administered after a first composition) is 0.1-fold, 1-fold, 5-fold, 10-fold, 100-fold, or 1000-fold higher than an amount of the polyribonucleotide (e.g., circular polyribonucleotide, linear polyribonucleotide) of a first composition. In some embodiments, an amount of the polyribonucleotide (e.g., circular polyribonucleotide, linear polyribonucleotide) of a second composition is from 0.1-fold to 1000-fold lower than an amount of the polyribonucleotide (e.g., circular polyribonucleotide, linear polyribonucleotide) of a first composition. In some embodiments, an amount of the polyribonucleotide (e.g., circular polyribonucleotide, linear polyribonucleotide) of a second composition is 0.1-fold, 1-fold, 5-fold, 10-fold, 100-fold, or 1000-fold lower than an amount of the polyribonucleotide (e.g., circular polyribonucleotide, linear polyribonucleotide) of a first composition. In some embodiments, an amount of the nucleic acid molecule (e.g., circular polyribonucleotide, linear polyribonucleotide) of a subsequent composition (e.g., a composition administered after a first composition) is 0.1-fold, 1-fold, 5-fold, 10-fold, 100-fold, or 1000-fold lower than an amount of the polyribonucleotide (e.g., circular polyribonucleotide, linear polyribonucleotide) of a first composition. In some embodiments, an amount of the polyribonucleotide (e.g., circular polyribonucleotide, linear polyribonucleotide) of a subsequent composition (e.g., after a first composition of an amount of polyribonucleotide (e.g., circular polyribonucleotide, linear polyribonucleotide)) is from 0.1-fold to 1000-fold higher or lower than an amount of the polyribonucleotide (e.g., circular polyribonucleotide, linear polyribonucleotide) of a first composition. In some embodiments, an amount of the polyribonucleotide (e.g., circular polyribonucleotide, linear polyribonucleotide) of a subsequent composition (e.g., after a first composition of an amount of polyribonucleotide (e.g., circular polyribonucleotide, linear polyribonucleotide)) is 0.1-fold, 1-fold, 5-fold, 10-fold, 100-fold, or 1000-fold higher or lower than an amount of the polyribonucleotide (e.g., circular polyribonucleotide, linear polyribonucleotide) of a first composition. For example, a first composition comprises 1-fold polyribonucleotide molecule (e.g., circular polyribonucleotide), a second composition comprises 5-fold polyribonucleotide (e.g., circular polyribonucleotide) compared to the first composition, and a third composition comprises 0.2-fold polyribonucleotide (e.g., circular polyribonucleotide) compared to the first composition. In some embodiments, the second composition comprises at least 5-fold polyribonucleotide (e.g., circular polyribonucleotide) compared to an amount of polyribonucleotide (e.g., circular polyribonucleotide) of a first composition.

In some embodiments, the first composition comprises a higher amount of the polyribonucleotide (e.g., circular polyribonucleotide) than the second composition. In some embodiments, the first composition comprises a higher amount of the polyribonucleotide (e.g., circular polyribonucleotides) than the third, fourth, fifth, sixth, seventh, eighth, ninth, or tenth composition.

In some embodiments, the plurality (e.g., two or more) of compositions of a polyribonucleotide (e.g., circular polyribonucleotide, linear polyribonucleotide) that are administered in a multiple dosing regimen as described herein, are the same compositions. In some embodiments, the plurality (e.g., two or more) of compositions of a polyribonucleotide (e.g., circular polyribonucleotide, linear polyribonucleotide) that are administered in a multiple dosing regimen as described herein, are different compositions. In some embodiments, the same compositions comprise the polyribonucleotides (e.g., circular polyribonucleotides, linear polyribonucleotides). In some embodiments, the different compositions comprise the polyribonucleotides (e.g., circular polyribonucleotides, linear polyribonucleotides), or a combination thereof.

A composition of the polyribonucleotide (e.g., a circular polyribonucleotide, linear polyribonucleotide) disclosed herein can induce a response in a subject.

In some embodiments, in a multiple dosing regimen, the method of administering the polyribonucleotide (e.g., circular polyribonucleotide, linear polyribonucleotide) provided herein includes administering to a subject in need thereof the polyribonucleotide for multiple times (multiple doses), e.g., at least 3, 4, 5, 6, 7, 8, 9, 10, 12, 15, 20, 30, 40, 50, 60, 100, 150, 200, or 500 times, with an interval of from 1 day to 56 days, such as about 49 days, 42 days, 35 days, 28 days, 21 days, 14 days, or 7 days. In some embodiments, in a multiple dosing regimen, the method provided herein includes administering to a subject in need thereof the polyribonucleotide for at least 3 times, with an interval of about 7 days. In some embodiments, in a subject that receives administration of multiple doses of the polyribonucleotide (e.g., at least 3, 4, 5, 6, 7, 8, or 9 doses) provided herein, a level of the encoded polypeptide is maintained at a level with variation of less than 50%, 40%, 30%, 20%, or 10% for a period of longer than 1, 2, 3, 4, 5, 6, 7, 8, 9, 10, 11, 12, 13, 14, 15, 16, 18, or 20 weeks after the last dose. In some embodiments, in a subject that receives administration of multiple doses of the polyribonucleotide (e.g., at least 3, 4, 5, 6, 7, 8, or 9 doses) provided herein, a level of the encoded polypeptide is maintained at a first level for a period of longer than 1, 2, 3, 4, 5, 6, 7, 8, 9, 10, 11, 12, 13, 14, 15, 16, 18, 19, or 20 weeks after the second, third, fourth, fifth, sixth, seventh, eight, or the last dose, wherein the first level is higher than a level of the encoded polypeptide measured shortly after the first dose (e.g., measured about 12, 24, 36, or 48 hours after the first dose). In some embodiments, in a subject that receives administration of multiple doses of the polyribonucleotide (e.g., at least 3 doses) provided herein with an interval of about 7 days, a level of the encoded polypeptide is maintained at a first level for a period of longer than 8, 9, 10, 11, 12, 13, 14, 15, 16, 17, 18, 19, or 20 weeks after the second, third, fourth, fifth, sixth, seventh, eight, or the last dose, wherein the first level is higher than a level of the encoded polypeptide measured shortly after the first dose (e.g., measured about 12, 24, 36, or 48 hours after the first dose).

### Methods of Delivery

A circular polyribonucleotide as described herein may be included in a composition (e.g., pharmaceutical or agricultural composition) with a carrier or without a carrier. The linear polyribonucleotide as described herein may be included in a composition (e.g., pharmaceutical or agricultural composition) with a carrier or without a carrier.

Pharmaceutical compositions described herein may be formulated for example including a carrier, such as a pharmaceutical carrier and/or a polymeric carrier, e.g., a liposome, and delivered by known methods to a subject in need thereof (e.g., a human or non-human agricultural or domestic animal, e.g., cattle, dog, cat, horse, poultry). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

Methods of delivery are also described, e.g., in Gori et al., Delivery and Specificity of CRISPR/Cas9 Genome Editing Technologies for Human Gene Therapy. Human Gene Therapy. July 2015, 26(7):443-451. doi:10.1089/hum.2015.074; and Zuris et al. Cationic lipid-mediated delivery of proteins enables efficient protein-based genome editing in vitro and in vivo. Nat Biotechnol. 2014 Oct. 30; 33(1):73-80.

Pharmaceutical compositions can be sterile and stable under the conditions of manufacture and storage. The composition can be formulated as a solution, microemulsion, liposome, or other ordered structure suitable to high drug concentration. Examples of suitable aqueous and non-aqueous compositions which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Sterile injectable solutions can be prepared by incorporating the active compound (e.g., a polyribonucleotide described herein (e.g., circular polyribonucleotide or linear polyribonucleotide)) in the required amount in an appropriate solvent with one or a combination of ingredients e.g., as described herein, as required, followed by sterilization microfiltration. Generally, dispersions are prepared by incorporating the active compound into a sterile vehicle that contains a basic dispersion medium and the required other ingredients e.g., from those described herein. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Polyribonucleotides (e.g., circular polyribonucleotides, linear polyribonucleotides) of the disclosure can be prepared in a composition that protects against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are generally known to those skilled in the art. A composition of the disclosure can be, for example, an immediate release form or a controlled release formulation. An immediate release formulation can be formulated to allow the compounds (e.g., agents, such as a circular polyribonucleotide or linear polyribonucleotide) to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

Pharmaceutical formulations can include aqueous solutions of the active compounds (e.g., polyribonucleotides (e.g., circular polyribonucleotide or linear polyribonucleotide) in water soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the agents to allow for the preparation of highly concentrated solutions. The active ingredient can be in powder form for constitution with a suitable vehicle, for example, sterile pyrogen-free water, before use.

Methods for the preparation of compositions comprising the polyribonucleotides described herein include formulating the polyribonucleotides with one or more inert, pharmaceutically acceptable excipients or carriers to form a solid, semi-solid, or liquid composition. Solid compositions include, for example, powders, dispersible granules, and cachets. Liquid compositions include, for example, solutions in which a polyribonucleotide is dissolved, emulsions comprising a polyribonucleotide, or a solution containing liposomes, micelles, or nanoparticles comprising a polyribonucleotide as disclosed herein. Semi-solid compositions include, for example, gels, suspensions, and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically acceptable additives.

Non-limiting examples of dosage forms suitable for use in the disclosure include liquid, powder, gel, nanosuspension, nanoparticle, microgel, aqueous or oily suspensions, emulsion, and any combination thereof.

In some embodiments, the formulation contains a thermal stabilizer, such as a sugar or sugar alcohol, for example, sucrose, sorbitol, glycerol, trehalose, or mannitol, or any combination thereof. In some embodiments, the stabilizer is a sugar. In some embodiments, the sugar is sucrose, mannitol, or trehalose.

In some embodiments, polyribonucleotides (e.g., circular polyribonucleotides, linear polyribonucleotides) are delivered in a naked delivery formulation. A naked delivery formulation delivers a polyribonucleotide (e.g., a circular polyribonucleotide or linear polyribonucleotide) to a cell without the aid of a carrier and without covalent modification of the polyribonucleotide (e.g., circular polyribonucleotide or linear polyribonucleotide) or partial or complete encapsulation of the polyribonucleotide (e.g., circular polyribonucleotide or linear polyribonucleotide).

In some embodiments, the circular polyribonucleotide molecule as described herein is delivered to a cell, tissue, or subject, includes administering the composition (e.g., pharmaceutical or agricultural composition) as described herein to the cell, tissue, or subject.

In some embodiments, the method of delivering is an in vivo method. For example, a method of delivery of a circular polyribonucleotide as described herein includes parenterally administering to a subject in need thereof, the composition, drug substance, or drug product as described herein to the subject in need thereof. As another example, a method of delivering a circular polyribonucleotide to a cell or tissue of a subject, includes administering parenterally to the cell or tissue the composition, drug substance, or drug product as described herein. In some embodiments, the circular polyribonucleotide is in an amount effective to elicit a biological response in the subject. In some embodiments, the circular polyribonucleotide is an amount effective to have a biological effect on the cell or tissue in the subject.

In some embodiments, the composition, drug substance, or drug product is administered parenterally. In some embodiments, the composition, drug substance, or drug product is administered intravenously, intraarterially, intraperitoneally, intradermally, intracranially, intrathecally, intralymphatically, subcutaneously, or intramuscularly. In some embodiments, parenteral administration is intravenously, intramuscularly, ophthalmically, subcutaneously, intradermally, or topically.

In some embodiments, the composition, drug substance, or drug product as described herein is administered intravenously. In some embodiments, the composition, drug substance, or drug product as described herein is administered intramuscularly. In some embodiments, the composition, drug substance, or drug product as described herein is administered subcutaneously. In some embodiments, the composition, drug substance, or drug product as described herein is administered topically. In some embodiments, the composition, drug substance, or drug product is administered intratracheally.

In some embodiments, the composition, drug substance, or drug product as described herein is administered orally. In some embodiments, the composition, drug substance, or drug product as described herein is administered nasally (e.g., intranasally). In some embodiments, the composition, drug substance, or drug product as described herein is administered by inhalation. In some embodiments, the composition, drug substance, or drug product as described herein is administered ophthalmically. In some embodiments, the composition, drug substance, or drug product as described herein is administered rectally. In some embodiments, the composition, drug substance, drug product is administered via intraocular administration, intracochlear (inner ear) administration, or intratracheal administration.

In some embodiments, the composition, drug substance, or drug product is administered by injection. The administration can be systemic administration or local administration. In some embodiments, the composition, drug substance, or drug product is administered via intraocular administration, intracochlear (inner ear) administration, or intratracheal administration. In some embodiments, the composition, drug substance, or drug product is administered prenatally, neonatally or postnatally. In some embodiments, any of the methods of delivery as described herein are performed with a carrier. In some embodiments, any methods of delivery as described herein are performed without the aid of a carrier in a naked delivery formulation.

In some embodiments, the cell is a eukaryotic cell. In some embodiments, the cell is a mammalian cell. In some embodiments, the cell is an animal cell. In some embodiments, the cell is an immune cell. In some embodiments, the cell is a plant cell. In some embodiments, the cell is an insect cell. In some embodiments, the tissue is a connective tissue, a muscle tissue, a nervous tissue, or an epithelial tissue. In some embodiments, the tissue is an organ (e.g., liver, lung, spleen, kidney, etc.). In some embodiments, the subject is a mammal.

In some embodiments, a pharmaceutical formulation disclosed herein can comprise: (i) a compound (e.g., circular polyribonucleotide) disclosed herein; (ii) a buffer; (iii) a non-ionic detergent; (iv) a tonicity agent; and (v) a stabilizer. In some embodiments, a pharmaceutical formulation disclosed herein can comprise: (i) a compound (e.g., linear polyribonucleotide) disclosed herein; (ii) a buffer; (iii) a non-ionic detergent; (iv) a tonicity agent; and (v) a stabilizer. In some embodiments, the pharmaceutical formulation disclosed herein is a stable liquid pharmaceutical formulation.

A host described herein can be exposed to any of the compositions described herein in any suitable manner that permits delivering or administering the composition to the insect. The polyribonucleotide may be delivered either alone or in combination with other active or inactive substances and may be applied by, for example, spraying, microinjection, through plants, pouring, dipping, in the form of concentrated liquids, gels, solutions, suspensions, sprays, powders, pellets, briquettes, bricks and the like, formulated to deliver an effective concentration of the polyribonucleotide.

Amounts and locations for application of the compositions described herein are generally determined by the habits of the host, the lifecycle stage at which the microorganisms of the host can be targeted by the polyribonucleotide, the site where the application is to be made, and the physical and functional characteristics of the polyribonucleotide. The polyribonucleotides described herein may be administered to the insect by oral ingestion but may also be administered by means which permit penetration through the cuticle or penetration of the insect respiratory system.

In some instances, the insect can be simply “soaked” or “sprayed” with a solution including the polyribonucleotide. Alternatively, the polyribonucleotide can be linked to a food component (e.g., comestible) of the insect for ease of delivery and/or in order to increase uptake of the polyribonucleotide by the insect. Methods for oral introduction include, for example, directly mixing a polyribonucleotide with the insect's food, spraying the polyribonucleotide in the insect's habitat or field, as well as engineered approaches in which a species that is used as food is engineered to express a polyribonucleotide, then fed to the insect to be affected. In some instances, for example, the polyribonucleotide composition can be incorporated into, or overlaid on the top of, the insect's diet. For example, the polyribonucleotide composition can be sprayed onto a field of crops which an insect inhabits.

In some instances, the composition is sprayed directly onto a plant e.g., crops, by e.g., backpack spraying, aerial spraying, crop spraying/dusting etc. In instances where the polyribonucleotide is delivered to a plant, the plant receiving the polyribonucleotide may be at any stage of plant growth. For example, formulated polyribonucleotide s can be applied as a seed-coating or root treatment in early stages of plant growth or as a total plant treatment at later stages of the crop cycle. In some instances, the polyribonucleotide may be applied as a topical agent to a plant, such that the host insect ingests or otherwise comes in contact with the plant upon interacting with the plant.

Further, the polyribonucleotide may be applied (e.g., in the soil in which a plant grows, or in the water that is used to water the plant) as a systemic agent that is absorbed and distributed through the tissues (e.g., stems or leafs) of a plant or animal host, such that an insect feeding thereon will obtain an effective dose of the polyribonucleotide. In some instances, plants or food organisms may be genetically transformed to express the polyribonucleotide such that a host feeding upon the plant or food organism will ingest the polyribonucleotide.

Delayed or continuous release can also be accomplished by coating the polyribonucleotide or a composition with the polyribonucleotide(s) with a dissolvable or bio-erodible coating layer, such as gelatin, which coating dissolves or erodes in the environment of use, to then make the polyribonucleotide available, or by dispersing the agent in a dissolvable or erodible matrix. Such continuous release and/or dispensing means devices may be advantageously employed to consistently maintain an effective concentration of one or more of the polyribonucleotide s described herein in a specific host habitat.

The polyribonucleotide can also be incorporated into the medium in which the insect grows, lives, reproduces, feeds, or infests. For example, a polyribonucleotide can be incorporated into a food container, feeding station, protective wrapping, or a hive. For some applications the polyribonucleotide may be bound to a solid support for application in powder form or in a “trap” or “feeding station.” As an example, for applications where the composition is to be used in a trap or as bait for a particular host insect, the compositions may also be bound to a solid support or encapsulated in a time-release material. For example, in instances where the host is a honeybee, the compositions described herein can be administered by delivering the composition to a honeybee hive or at least one habitat where a honeybee grows, lives, reproduces, or feeds.

### Formulations

In some embodiments of the present disclosure a polyribonucleotide (e.g., a circular polyribonucleotide, linear polyribonucleotide) described herein is formulated in composition, e.g., a composition for delivery to a cell, a plant, an invertebrate animal, a non-human vertebrate animal, or a human subject, e.g., an agricultural or pharmaceutical composition. In some embodiments, the polyribonucleotide (e.g., circular polyribonucleotide, linear polyribonucleotide) is formulated in a pharmaceutical composition. In some embodiments, a composition includes a polyribonucleotide (e.g., a circular polyribonucleotide or a linear polyribonucleotide) and a diluent, a carrier, an adjuvant, or a combination thereof. In a particular embodiment, a composition includes a polyribonucleotide (e.g., a circular polyribonucleotide or a linear polyribonucleotide) described herein and a carrier or a diluent free of any carrier. In some embodiments, a composition including a polyribonucleotide (e.g., a circular polyribonucleotide or a linear polyribonucleotide) with a diluent free of any carrier is used for naked delivery of the polyribonucleotide to a subject.

Formulations of pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In some embodiments, a pharmaceutical formulation disclosed herein can include: (i) a compound (e.g., circular polyribonucleotide) disclosed herein; (ii) a buffer; (iii) a non-ionic detergent; (iv) a tonicity agent; and/or (v) a stabilizer. In some embodiments, the pharmaceutical formulation disclosed herein is a stable liquid pharmaceutical formulation. In some embodiments, the pharmaceutical formulation disclosed herein includes protamine or a protamine salt (e.g., protamine sulfate).

Pharmaceutical compositions may optionally include one or more additional active substances, e.g., therapeutically and/or prophylactically active substances. Pharmaceutical compositions may optionally include an inactive substance that serves as a vehicle or medium for the compositions described herein (e.g., compositions including circular polyribonucleotides, such as any one of the inactive ingredients approved by the United States Food and Drug Administration (FDA) and listed in the Inactive Ingredient Database). Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference). Non-limiting examples of an inactive substance include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof.

Although the descriptions of compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as parrots, poultry, chickens, ducks, geese, hens or roosters, and/or turkeys; zoo animals, such as felines; non-mammal animals, such as reptiles, fish, amphibians, etc.

The compositions described herein may be formulated either in pure form or together with one or more additional agents (such as excipient, delivery vehicle, carrier, diluent, stabilizer, etc.) to facilitate application or delivery of the compositions. Examples of suitable excipients and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil.

In some instances, the composition includes a delivery vehicle or carrier. In some instances, the delivery vehicle includes an excipient. Exemplary excipients include, but are not limited to, solid or liquid carrier materials, solvents, stabilizers, slow-release excipients, colorings, and surface-active substances (surfactants). In some instances, the delivery vehicle is a stabilizing vehicle. In some instances, the stabilizing vehicle includes a stabilizing excipient. Exemplary stabilizing excipients include, but are not limited to, epoxidized vegetable oils, antifoaming agents, e.g., silicone oil, preservatives, viscosity regulators, binding agents and tackifiers. In some instances, the composition is microencapsulated in a polymer bead delivery vehicle. In some instances, the delivery vehicle contains a pH buffer. In some instances, the composition is formulated to have a pH in the range of about 4.5 to about 9.0, including for example pH ranges of about any one of 5.0 to about 8.0, about 6.5 to about 7.5, or about 6.5 to about 7.0.

Depending on the intended objectives and prevailing circumstances, the composition may be formulated into emulsifiable concentrates, suspension concentrates, directly sprayable or dilutable solutions, coatable pastes, diluted emulsions, spray powders, soluble powders, dispersible powders, wettable powders, dusts, granules, encapsulations in polymeric substances, microcapsules, foams, aerosols, carbon dioxide gas preparations, tablets, resin preparations, paper preparations, nonwoven fabric preparations, or knitted or woven fabric preparations. In some instances, the composition is a liquid. In some instances, the composition is a solid. In some instances, the composition is an aerosol, such as in a pressurized aerosol can. In some instances, the composition is present in the waste (such as feces) of the pest. In some instances, the composition is present in or on a live pest.

In some instances, the delivery vehicle is the food or water of the host. In other instances, the delivery vehicle is a food source for the host. In some instances, the delivery vehicle is a food bait for the host. In some instances, the composition is a comestible agent consumed by the host. In some instances, the composition is delivered by the host to a second host and consumed by the second host. In some instances, the composition is consumed by the host or a second host, and the composition is released to the surrounding of the host or the second host via the waste (such as feces) of the host or the second host. In some instances, the composition includes at least any of 0.1%, 0.5%, 1%, 5%, 10%, 20%, 30%, 40%, 50%, 60%, 70%, 80%, 90%, 95%, or more active ingredients (such as phage, lysin or bacteriocin). In some instances, the concentrated agents are preferred as commercial products, the final user normally uses diluted agents, which have a substantially lower concentration of active ingredient.

Any of the formulations described herein may be used in the form of a bait, a coil, an electric mat, a smoking preparation, a fumigant, or a sheet.

### Preservatives

A composition (e.g., pharmaceutical or agricultural composition) provided herein can include material for a single administration, or can include material for multiple administrations (e.g., a “multidose” kit). The polyribonucleotide can be present in either linear or circular form. The composition (e.g., pharmaceutical or agricultural composition) can include one or more preservatives such as thiomersal or 2-phenoxyethanol. Preservatives can be used to prevent microbial contamination during use. Suitable preservatives include: benzalkonium chloride, thimerosal, chlorobutanol, methyl paraben, propyl paraben, phenylethyl alcohol, edetate disodium, sorbic acid, Onamer M, or other agents known to those skilled in the art. In ophthalmic products, e.g., such preservatives can be employed at a level of from 0.004% to 0.02%. In the compositions described herein the preservative, e.g., benzalkonium chloride, can be employed at a level of from 0.001% to less than 0.01%, e.g., from 0.001% to 0.008%, preferably about 0.005% by weight.

Polyribonucleotides can be susceptible to RNase that can be abundant in ambient environment. Compositions (e.g., pharmaceutical or agricultural compositions) provided herein can include reagents that inhibit RNase activity, thereby preserving the polyribonucleotide from degradation. In some cases, the composition (e.g., pharmaceutical or agricultural composition) includes any RNase inhibitor known to one skilled in the art. Alternatively or additionally, the polyribonucleotide, and cell-penetrating agent and/or diluents or carriers, vehicles, excipients, or other reagents in the composition provided herein can be prepared in RNase-free environment. The composition can be formulated in RNase-free environment.

In some cases, a composition provided herein can be sterile. The composition can be formulated as a sterile solution or suspension, in suitable vehicles, known in the art. The composition can be sterilized by conventional, known sterilization techniques, e.g., the composition can be sterile filtered.

### Salts

In some cases, a composition (e.g., pharmaceutical or agricultural composition) provided herein includes one or more salts. For controlling the tonicity, a physiological salt such as sodium salt can be included in a composition provided herein. Other salts can include potassium chloride, potassium dihydrogen phosphate, disodium phosphate, and/or magnesium chloride, or the like. In some embodiments, a pharmaceutical composition is formulated with one or more pharmaceutically acceptable salts. The one or more pharmaceutically acceptable salts can include those of the inorganic ions, such as, for example, sodium, potassium, calcium, magnesium ions, and the like. Such salts can include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid, or maleic acid. The polyribonucleotide can be present in either linear or circular form.

### Buffers/pH

A composition (e.g., pharmaceutical or agricultural composition) provided herein can include one or more buffers, such as a Tris buffer; a borate buffer; a succinate buffer; a histidine buffer (e.g., with an aluminum hydroxide adjuvant); or a citrate buffer. Buffers, in some cases, are included in the 5-20 mM range.

A composition (e.g., pharmaceutical or agricultural composition) provided herein can have a pH between about 5.0 and about 8.5, between about 6.0 and about 8.0, between about 6.5 and about 7.5, or between about 7.0 and about 7.8. The composition (e.g., pharmaceutical, veterinary, or agricultural composition) can have a pH of about 7. The polyribonucleotide can be present in either linear or circular form.

### Detergents/Surfactants

A composition (e.g., pharmaceutical or agricultural composition) provided herein can include one or more detergents and/or surfactants, depending on the intended administration route, e.g., polyoxyethylene sorbitan esters surfactants (commonly referred to as “Tweens”), e.g., polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxinols, which can vary in the number of repeating ethoxy (oxy-1,2-ethanediyl) groups, e.g., octoxynol-9 (Triton X-100, or t-octylphenoxypolyethoxyethanol); (octylphenoxy) polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as “SPANs”), such as sorbitan trioleate (Span 85) and sorbitan monolaurate, an octoxynol (such as octoxynol-9 (Triton X-100) or t-octylphenoxypolyethoxyethanol), a cetyl trimethyl ammonium bromide (“CTAB”), or sodium deoxycholate. The one or more detergents and/or surfactants can be present only at trace amounts. In some cases, the composition can include less than 1 mg/ml of each of octoxynol-10 and polysorbate 80. Non-ionic surfactants can be used herein. Surfactants can be classified by their “HLB” (hydrophile/lipophile balance). In some cases, surfactants have a HLB of at least 10, at least 15, and/or at least 16. The polyribonucleotide can be present in either linear or circular form.

### Diluents

In some embodiments, a composition (e.g., pharmaceutical or agricultural composition) provided herein includes a circular polyribonucleotide and a diluent. In some embodiments, a composition (e.g., pharmaceutical or agricultural composition) provided herein includes a linear polyribonucleotide and a diluent.

In certain embodiments, a composition (e.g., pharmaceutical or agricultural composition) comprises a circular polyribonucleotide as described herein in a vesicle or other membrane-based carrier. In certain embodiments, a composition (e.g., pharmaceutical or agricultural composition) comprises a linear polyribonucleotide as described herein in a vesicle or other membrane-based carrier.

A diluent can be a non-carrier excipient. A non-carrier excipient serves as a vehicle or medium for a composition, such as a polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. A non-carrier excipient can be any inactive ingredient suitable for administration to a non-human animal, for example, suitable for veterinary use. Modification of compositions suitable for administration to humans in order to render the compositions suitable for administration to various animals is well understood, and the ordinarily skilled veterinary pharmacologist can design and/or perform such modification with merely ordinary, if any, experimentation.

In some embodiments, the polyribonucleotide (e.g., circular polyribonucleotide or linear polyribonucleotide) is delivered as a naked delivery formulation, such as including a diluent. A naked delivery formulation delivers a polyribonucleotide (e.g., circular polyribonucleotide or linear polyribonucleotide), to a cell without the aid of a carrier and without covalent modification of the polyribonucleotide or partial or complete encapsulation of the polyribonucleotide.

A naked delivery formulation is a formulation that is free from a carrier and wherein the polyribonucleotide is without a covalent modification that binds a moiety that aids in delivery to a cell or without partial or complete encapsulation of the polyribonucleotide. In some embodiments, a polyribonucleotide without a covalent modification that binds a moiety that aids in delivery to a cell is a polyribonucleotide that is not covalently bound to a moiety, such as a protein, small molecule, a particle, a polymer, or a biopolymer that aids in delivery to a cell. A polyribonucleotide without covalent modification that binds a moiety that aids in delivery to a cell does not contain a modified phosphate group. For example, a polyribonucleotide without a covalent modification that binds a moiety that aids in delivery to a cell does not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation is free of any or all transfection reagents, cationic carriers, carbohydrate carriers, nanoparticle carriers, or protein carriers. In some embodiments, a naked delivery formulation is free from phytoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1 (2,3-dioleoyloxy) propyl]-N, N, N-trimethylammonium chloride (DOTMA), I-[2-(oleoyloxy)ethyl]-2-oleyl-3-(2-hydroxyethyl) imidazolinium chloride (DOTIM), 2,3-dioleyloxy-N-[2 (sperminecarboxamido)ethyl]-N,N-dimethyl-I-propanaminium trifluoroacetate (DOSPA), 3B—[N—(N\N′-Dimethylaminoethane)-carbamoyl] Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

In certain embodiments, a naked delivery formulation includes a non-carrier excipient. A non-carrier excipient serves as a vehicle or medium for a polyribonucleotide (e.g., circular polyribonucleotide, linear polyribonucleotide) described herein. In some embodiments, a non-carrier excipient includes an inactive ingredient that does not exhibit a cell-penetrating effect. In some embodiments, a non-carrier excipient includes a buffer, for example PBS. In some embodiments, a non-carrier excipient is a solvent, a non-aqueous solvent, a dispersion media, a diluent, a suspension aid, a surface-active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil, or mixtures thereof. In some embodiments, a non-carrier excipient is a pharmaceutically acceptable excipient. A non-carrier excipient can be any one of the inactive ingredients approved by the United States Food and Drug Administration (FDA) and listed in the Inactive Ingredient Database that does not exhibit a cell-penetrating effect.

In some embodiments, a naked delivery formulation includes a diluent, such as a parenterally acceptable diluent. A diluent e.g., a parenterally acceptable diluent) may be a liquid diluent or a solid diluent. In some embodiments, a diluent e.g., a parenterally acceptable diluent) is an RNA solubilizing agent, a buffer, or an isotonic agent. Examples of an RNA solubilizing agent include water, ethanol, methanol, acetone, formamide, and 2-propanol. Examples of a buffer include 2-(N-morpholino) ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino) propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

In some embodiments, the formulation includes a cell-penetrating agent. In some embodiments, the formulation is a topical formulation and includes a cell-penetrating agent. The cell-penetrating agent can include organic compounds such as alcohols having one or more hydroxyl function groups. In some cases, the cell-penetrating agent includes an alcohol such as, but not limited to, monohydric alcohols, polyhydric alcohols, unsaturated aliphatic alcohols, and alicyclic alcohols. The cell-penetrating agent can include one or more of methanol, ethanol, isopropanol, phenoxyethanol, triethanolamine, phenethyl alcohol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, or hydroxyethylcellulose. In certain embodiments, the cell-penetrating agent includes ethanol. The cell-penetrating agents can include any cell-penetrating agent in any amount or in any formulation as described in WO 2020/180751 or WO 2020/180752, which are hereby incorporated by reference in their entirety.

### Carriers

In some embodiments, a disclosure provides composition (e.g., a pharmaceutical or agricultural composition) provided herein includes any one of the polyribonucleotides described herein and a carrier.

In certain embodiments, a composition (e.g., pharmaceutical or agricultural composition) comprises a circular polyribonucleotide as described herein in a vesicle or other membrane-based carrier. In certain embodiments, a composition (e.g., pharmaceutical or agricultural composition comprises a linear polyribonucleotide as described herein in a vesicle or other membrane-based carrier.

In other embodiments, a composition (e.g., pharmaceutical or agricultural composition) includes the polyribonucleotide in or via a cell, vesicle, or other membrane-based carrier. In one embodiment, the composition (e.g., pharmaceutical or agricultural composition) includes the polyribonucleotide in liposomes or other similar vesicles. Liposomes are spherical vesicle structures composed of a uni- or multilamellar lipid bilayer surrounding internal aqueous compartments and an impermeable outer lipophilic phospholipid bilayer. Liposomes may be anionic, neutral, or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, JOURNAL OF DRUG DELIVERY, vol. 2011, Article ID 469679, 12 pages, 2011. doi: 10.1155/2011/469679 for review).

Vesicles can be made from several diverse types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, JOURNAL OF DRUG DELIVERY, vol. 2011, Article ID 469679, 12 pages, 2011. doi: 10.1155/2011/469679 for review). Extruded lipids can be prepared by extruding through filters of decreasing size, as described in Templeton et al., NATURE BIOTECH, 15:647-52, 1997, the teachings of which relating to extruded lipid preparation are incorporated herein by reference.

In certain embodiments, a composition (e.g., a pharmaceutical, veterinary, or agricultural composition) of the disclosure includes a polyribonucleotide and lipid nanoparticles, for example lipid nanoparticles described herein. Lipid nanoparticles are another example of a carrier that provides a biocompatible and biodegradable delivery system for a polyribonucleotide molecule as described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are a key component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi: 10.3390/nano7060122.

Additional non-limiting examples of carriers include carbohydrate carriers (e.g., an anhydride-modified phytoglycogen or glycogen-type material), protein carriers (e.g., a protein covalently linked to the polyribonucleotide), or cationic carriers (e.g., a cationic lipopolymer or transfection reagent). Non-limiting examples of carbohydrate carriers include phytoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1 (2,3-dioleoyloxy) propyl]-N, N,N-trimethylammonium chloride (DOTMA), I-[2-(oleoyloxy)ethyl]-2-oleyl-3-(2-hydroxyethyl) imidazolinium chloride (DOTIM), 2,3-dioleyloxy-N-[2 (sperminecarboxamido)ethyl]-N,N-dimethyl-I-propanaminium trifluoroacetate (DOSPA), 3B—[N—(N\N′-Dimethylaminoethane)-carbamoyl] Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high-density lipoprotein (HDL), or globulin.

Exosomes can also be used as drug delivery vehicles for polyribonucleotides described herein. For a review, see Ha et al. July 2016. ACTA PHARMACEUTICA SINICA B. Volume 6, Issue 4, Pages 287-296; https://doi.org/10.1016/j.apsb.2016.02.001.

Ex vivo differentiated red blood cells can also be used as a carrier for polyribonucleotides described herein. See, e.g., International Patent Publication Nos. WO2015/073587; WO2017/123646; WO2017/123644; WO2018/102740; WO2016/183482; WO2015/153102; WO2018/151829; WO2018/009838; Shi et al. 2014. Proc Natl Acad Sci USA. 111 (28): 10131-10136; U.S. Pat. No. 9,644,180; Huang et al. 2017. NATURE COMMUNICATIONS 8:423; Shi et al. 2014. PROC NATL ACAD SCI USA. 111 (28): 10131-136.

Fusosome compositions, e.g., as described in International Patent Publication No. WO2018/208728, can also be used as carriers to deliver a polyribonucleotide molecule described herein.

Virosomes and virus-like particles (VLPs) can also be used as carriers to deliver a polyribonucleotide molecule described herein to targeted cells.

Plant nanovesicles and plant messenger packs (PMPs), e.g., as described in International Patent Publication Nos. WO2011/097480, WO2013/070324, WO2017/004526, or WO2020/041784 can also be used as carriers to deliver polyribonucleotides described herein.

Microbubbles can also be used as carriers to deliver a polyribonucleotide molecule described herein. See, e.g., U.S. Pat. No. 7,115,583; Beeri, R. et al., CIRCULATION. 2002 Oct. 1; 106(14):1756-59; Bez, M. et al., NAT PROTOC. 2019 April; 14(4):1015-26; Hernot, S. et al., ADV DRUG DELIV REV. 2008 Jun. 30; 60(10):1153-66; Rychak, J. J. et al., ADV DRUG DELIV REV. 2014 June; 72:82-93. In some embodiments, microbubbles are albumin-coated perfluorocarbon microbubbles.

The carrier including the polyribonucleotides described herein may include a plurality of particles. The particles may have median article size of 30 to 700 nanometers (e.g., 30 to 50, 50 to 100, 100 to 200, 200 to 300, 300 to 400, 400 to 500, 500 to 600, 600 to 700, 100 to 500, 50 to 500, or 200 to 700 nanometers). The size of the particle may be optimized to favor deposition of the payload, including the polyribonucleotide into a cell. Deposition of the polyribonucleotide into certain cell types may favor different particle sizes. For example, the particle size may be optimized for deposition of the polyribonucleotide into immunogen presenting cells. The particle size may be optimized for deposition of the polyribonucleotide into dendritic cells. Additionally, the particle size may be optimized for depositions of the polyribonucleotide into draining lymph node cells.

### Lipid Nanoparticles

The compositions, methods, and delivery systems provided by the present disclosure may employ any suitable carrier or delivery modality described herein, including, in certain embodiments, lipid nanoparticles (LNPs). Lipid nanoparticles, in some embodiments, include one or more ionic lipids, such as non-cationic lipids (e.g., neutral or anionic, or zwitterionic lipids); one or more conjugated lipids (such as PEG-conjugated lipids or lipids conjugated to polymers described in Table 5 of International Patent Publication No. WO2019/217941; incorporated herein by reference in its entirety); one or more sterols (e.g., cholesterol).

Lipids that can be used in nanoparticle formations (e.g., lipid nanoparticles) include, for example those described in Table 4 of International Patent Publication No. WO2019/217941, which is incorporated by reference—e.g., a lipid-containing nanoparticle can include one or more of the lipids in Table 4 of International Patent Publication No. WO2019/217941. Lipid nanoparticles can include additional elements, such as polymers, such as the polymers described in Table 5 of International Patent Publication No. WO2019/217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy) propyl-1-0-(w-methoxy (polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of International Patent Publication No. WO2019/051289 (incorporated by reference), and combinations of the foregoing. Additional exemplary PEG-lipid conjugates are described, for example, in U.S. Pat. Nos. 5,885,613, 6,287,591, US2003/0077829, US2003/0077829, US2005/0175682, US2008/0020058, US 2011/0117125, US2010/0130588, US2016/0376224, US2017/0119904, US2018/0028664, and International Patent Publication No. WO2017/099823, the contents of all of which are incorporated herein by reference in their entirety.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al. (2020), dx.doi.org/10.1021/acs.nanolett.0c01386, incorporated herein by reference.

In some embodiments, the lipid particle includes an ionizable lipid, a non-cationic lipid, a conjugated lipid that inhibits aggregation of particles, and a sterol. The amounts of these components can be varied independently and to achieve desired properties. For example, in some embodiments, the lipid nanoparticle includes an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA (e.g., circular polyribonucleotide, linear polyribonucleotide)) described herein includes,

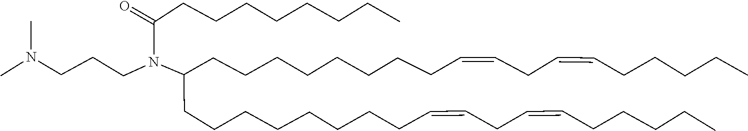 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments, an LNP including Formula (i) is used to deliver a polyribonucleotide (e.g., a circular polyribonucleotide, a linear polyribonucleotide) composition described herein to cells.

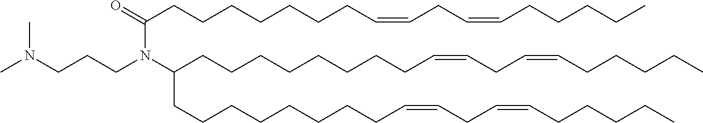 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments, an LNP including Formula (ii) is used to deliver a polyribonucleotide (e.g., a circular polyribonucleotide, a linear polyribonucleotide) composition described herein to cells.

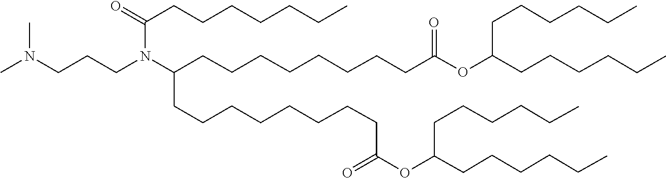 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments, an LNP including Formula (iii) is used to deliver a polyribonucleotide (e.g., a circular polyribonucleotide, a linear polyribonucleotide) composition described herein to cells.

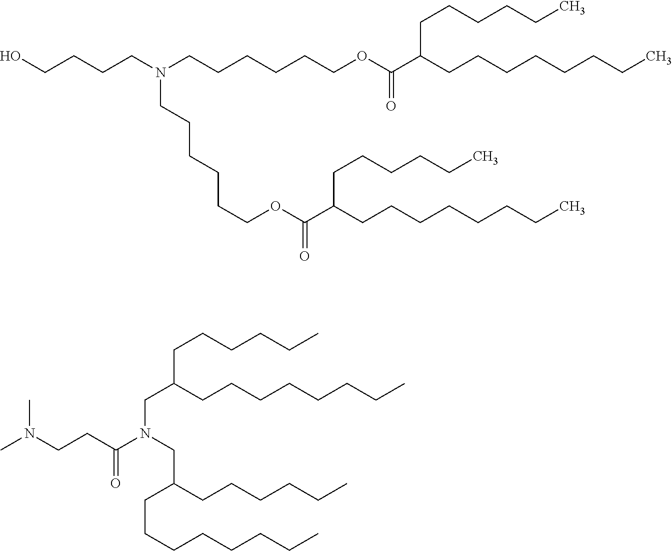

In some embodiments, an LNP including Formula (v) is used to deliver a polyribonucleotide (e.g., a circular polyribonucleotide, a linear polyribonucleotide) composition described herein to cells.

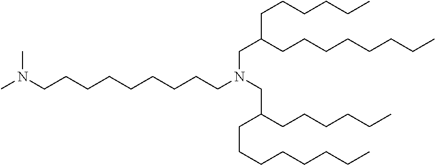 [SMILES: CCCCCCCCC(CCCCCC)CN(CCCCCCCCCN(C)C)CC(CCCCCC)CCCCCCCC]

In some embodiments, an LNP including Formula (vi) is used to deliver a polyribonucleotide (e.g., a circular polyribonucleotide, a linear polyribonucleotide) composition described herein to cells.

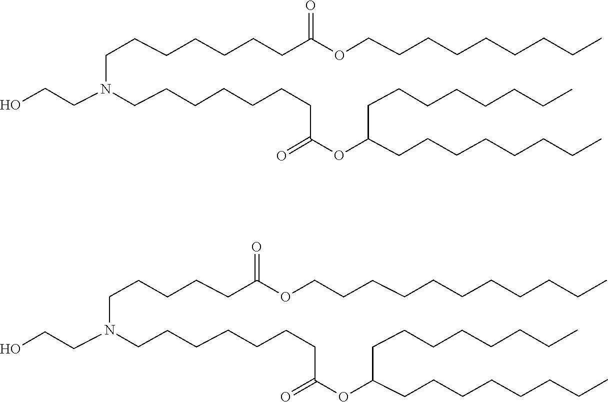 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments, an LNP including Formula (viii) is used to deliver a polyribonucleotide (e.g., a circular polyribonucleotide, a linear polyribonucleotide) composition described herein to cells.

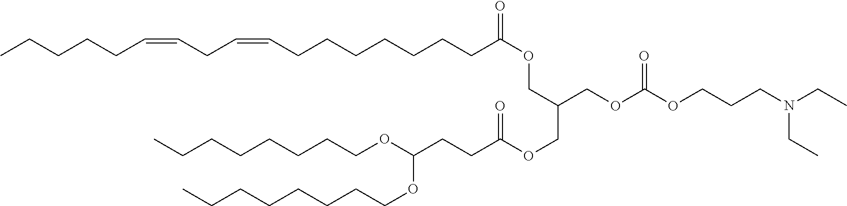 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments, an LNP including Formula (ix) is used to deliver a polyribonucleotide (e.g., a circular polyribonucleotide, a linear polyribonucleotide) composition described herein to cells.

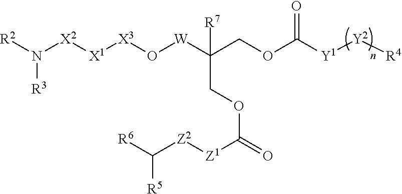

in which
X¹ is O, NR¹, or a direct bond, X² is C₂₋₅ alkylene, X³ is C(═O) or a direct bond, R¹ is H or Me, R³ is C₁₋₃ alkyl, R² is C₁₋₃ alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

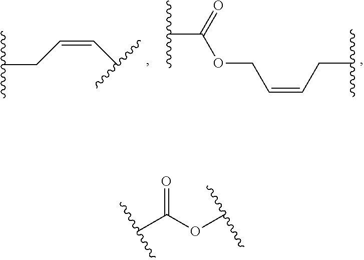 [SMILES: */C=C\C* ; *C/C=C\COC(*)=O ; *OC(*)=O]

(in either orientation), (in either orientation), (in either orientation),
n is 0 to 3, R⁴ is C₁-15 alkyl, Z¹ is C1-6 alkylene or a direct bond,
Z² is

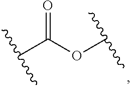 [SMILES: *OC(*)=O]

(in either orientation) or absent, provided that if Z¹ is a direct bond, Z² is absent;
R⁵ is C₅₋₉ alkyl or C6-10 alkoxy, R⁶ is C₅₋₉ alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene, (Y²)n-R⁴ is

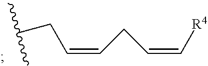

R⁴ is linear C5 alkyl, Z¹ is C2 alkylene, Z² is absent, W is methylene, and R⁷ is H, then R⁵ and R⁶ are not Cx alkoxy.

In some embodiments, an LNP including Formula (xii) is used to deliver a polyribonucleotide (e.g., a circular polyribonucleotide, a linear polyribonucleotide) composition described herein to cells.

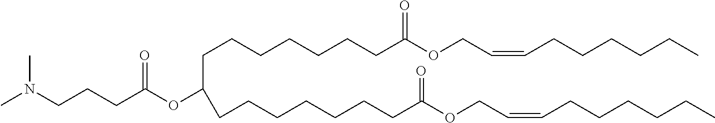 [SMILES: CCCCCC/C=C\COC(=O)CCCCCCCC(CCCCCCCC(=O)OC/C=C\CCCCCC)OC(=O)CCCN(C)C]

In some embodiments, an LNP including Formula (xi) is used to deliver a polyribonucleotide (e.g., a circular polyribonucleotide, a linear polyribonucleotide) composition described herein to cells.

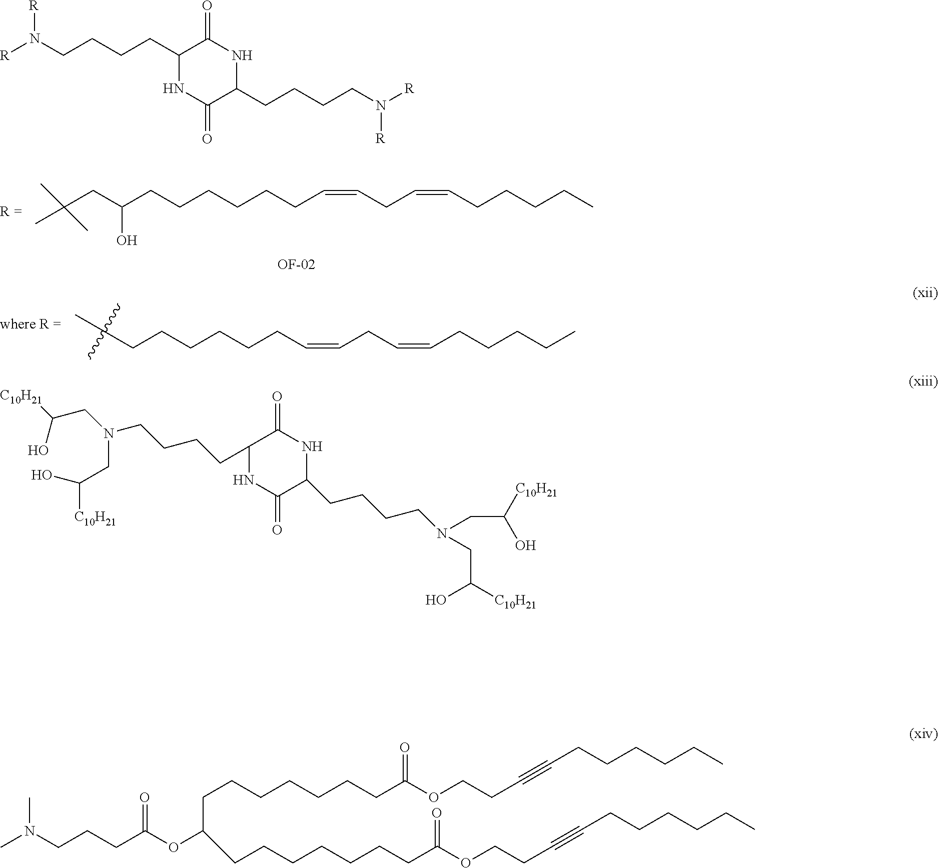

In some embodiments, an LNP includes a compound of Formula (xiii) and a compound of Formula (xiv).

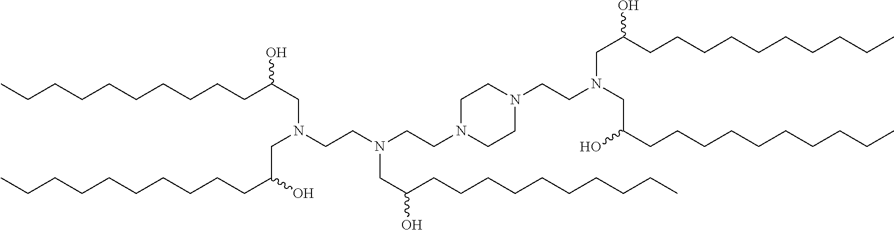 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, an LNP including Formula (xv) is used to deliver a polyribonucleotide (e.g., a circular polyribonucleotide, a linear polyribonucleotide) composition described herein to cells.

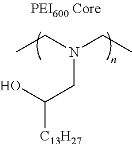

In some embodiments, an LNP including a formulation of Formula (xvi) is used to deliver a polyribonucleotide (e.g., a circular polyribonucleotide, a linear polyribonucleotide) composition described herein to cells.

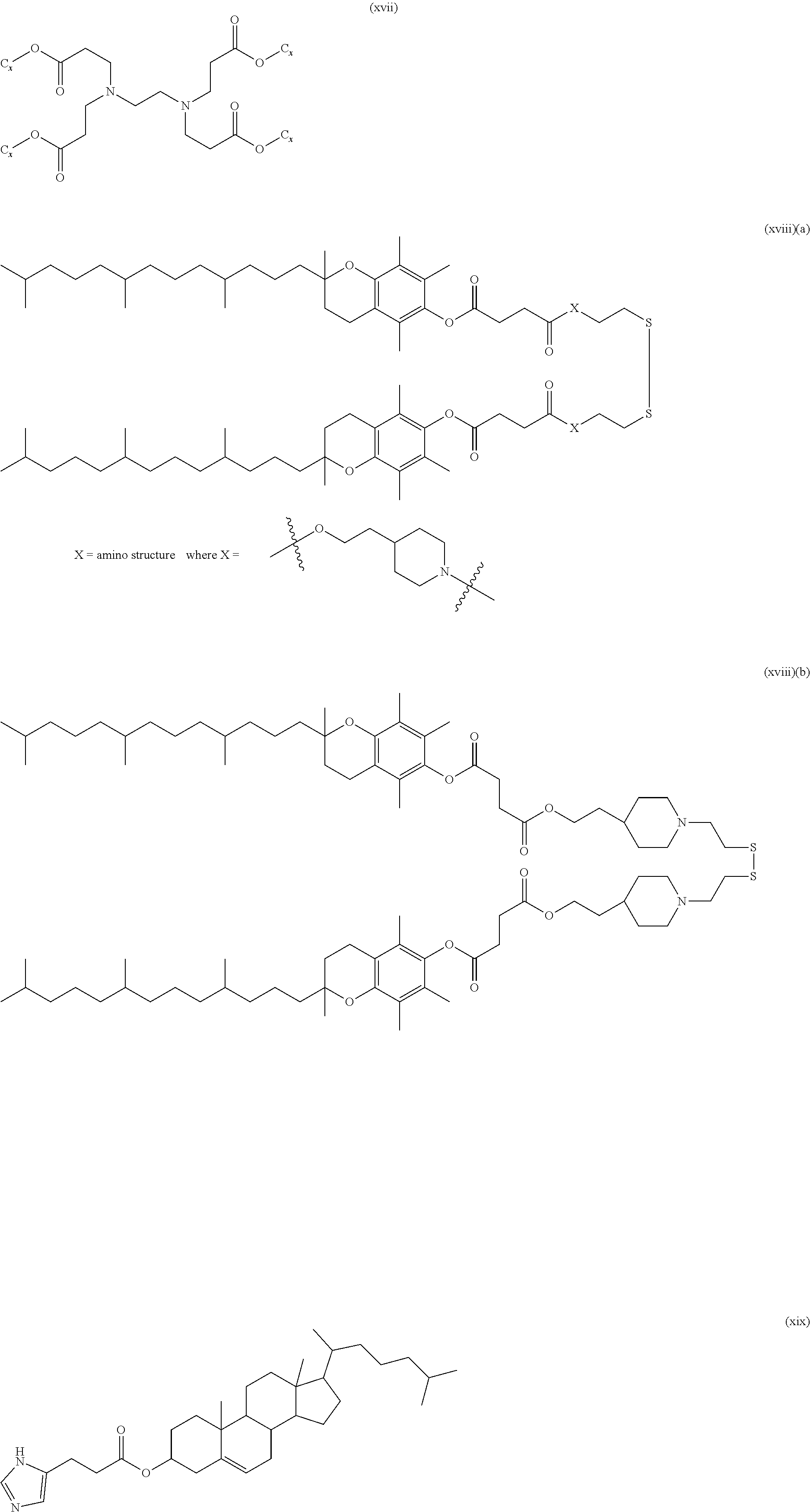

In some embodiments, a lipid compound used to form lipid nanoparticles for the delivery of compositions described herein, e.g., nucleic acid (e.g., RNA (e.g., circular polyribonucleotide, linear polyribonucleotide)) described herein is made by one of the following reactions:

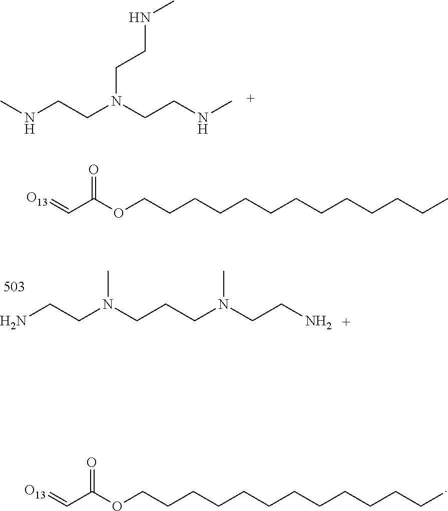

In some embodiments, an LNP including Formula (xxi) is used to deliver a polyribonucleotide (e.g., a circular polyribonucleotide, a linear polyribonucleotide) composition described herein to cells. In some embodiments, the LNP of Formula (xxi) is an LNP described by International Patent Publication No. WO2021/113777 (e.g., a lipid of Formula (1) such as a lipid of Table 1 of International Patent Publication No. WO2021/113777).

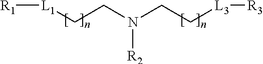

in which
each n is independently an integer from 2-15; L₁ and L₃ are each independently —OC(O)—* or —C(O)O—*, in which “*” indicates the attachment point to R₁ or R₃.
R₁ and R₅ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkyl sulfonyl, and alkyl sulfonealkyl; and
R₂ is selected from a group consisting of:

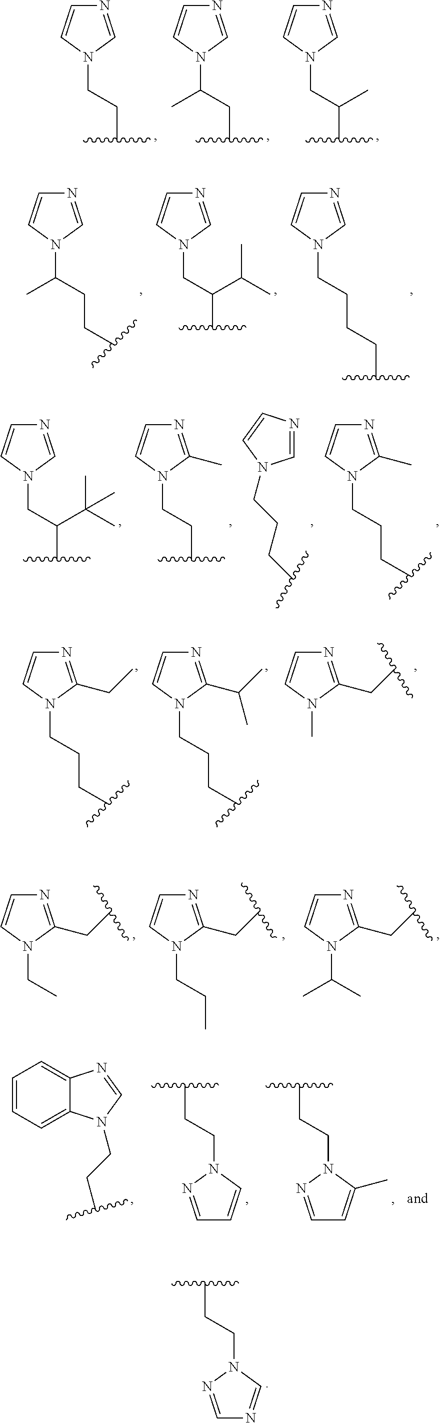

In some embodiments, an LNP including Formula (xxii) is used to deliver a polyribonucleotide (e.g., a circular polyribonucleotide, a linear polyribonucleotide) composition described herein to cells. In some embodiments, the LNP of Formula (xxii) is an LNP described by International Patent Publication No. WO2021/113777 (e.g., a lipid of Formula (2) such as a lipid of Table 2 of International Patent Publication No. WO2021/113777).

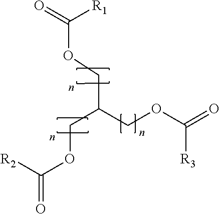

in which
each n is independently an integer from 1-15;
R₁ and R₂ are each independently selected from a group consisting of:

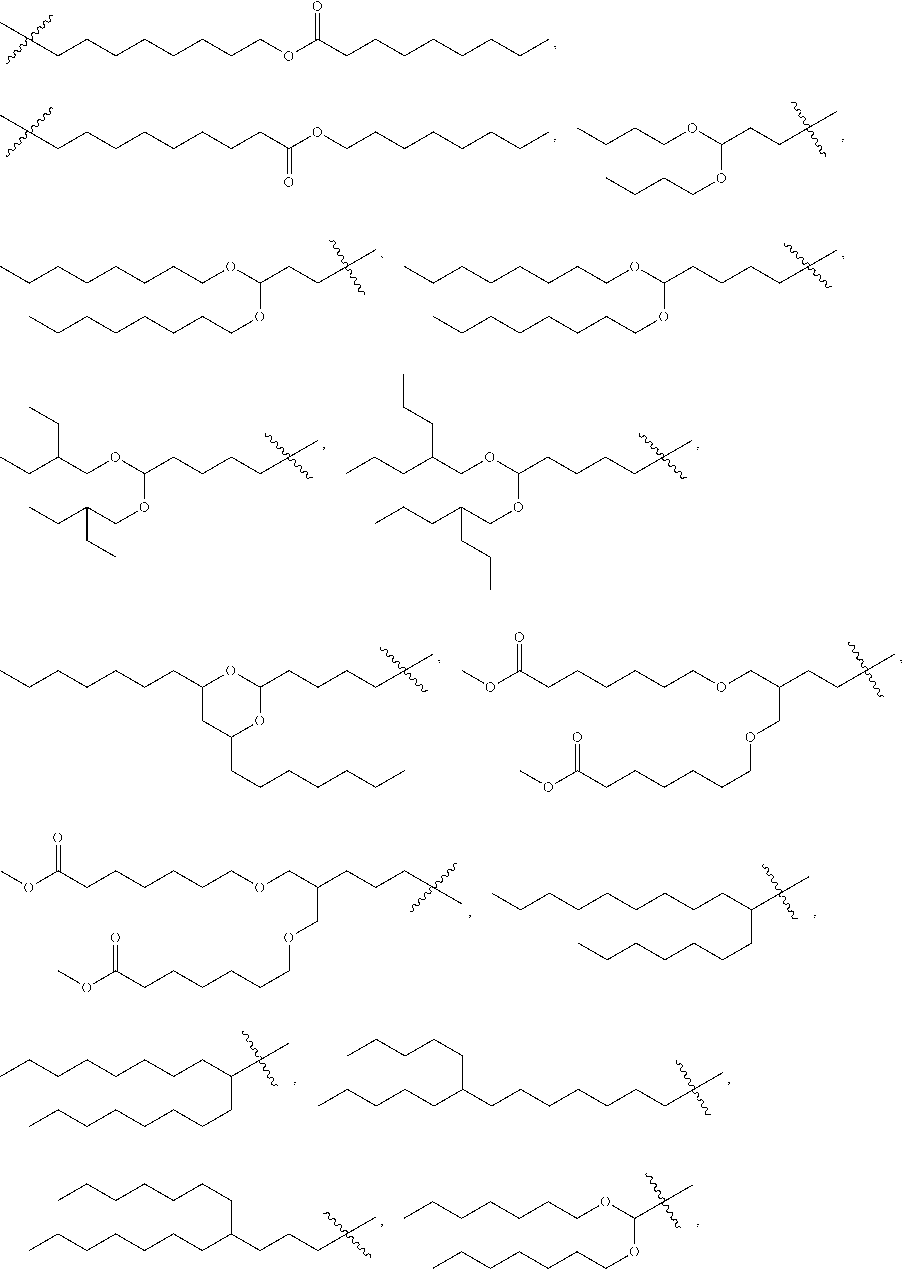

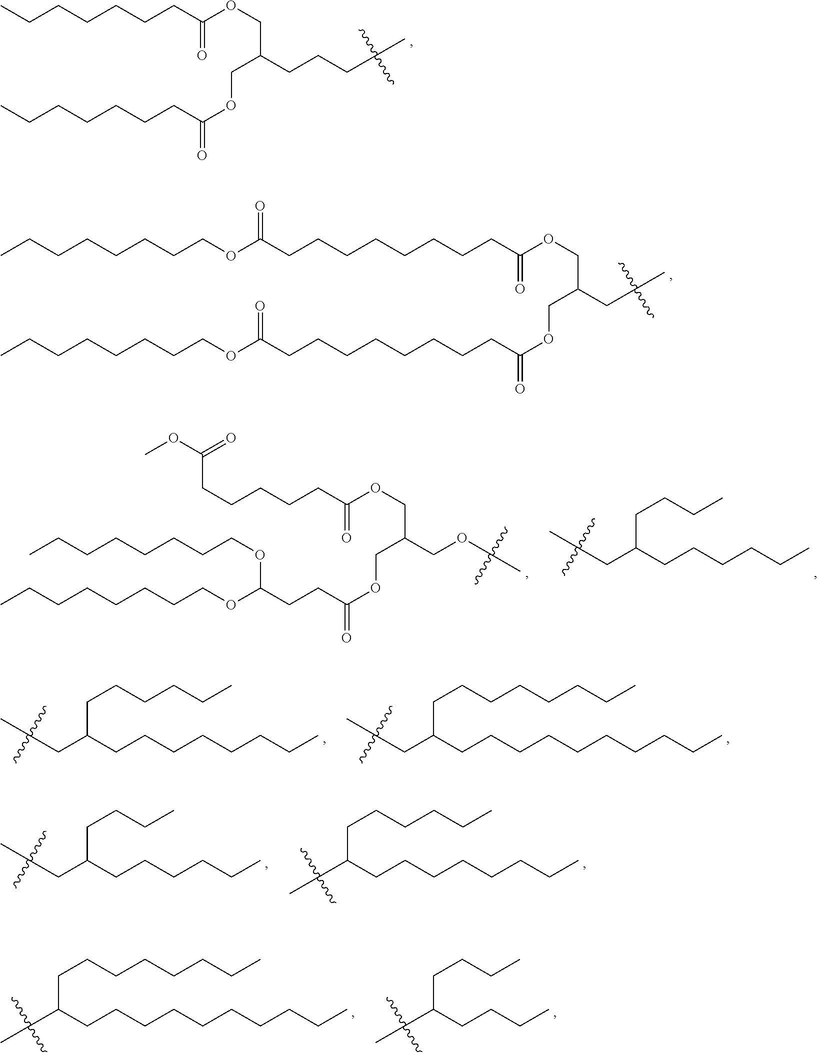

R₃ is selected from a group consisting of:

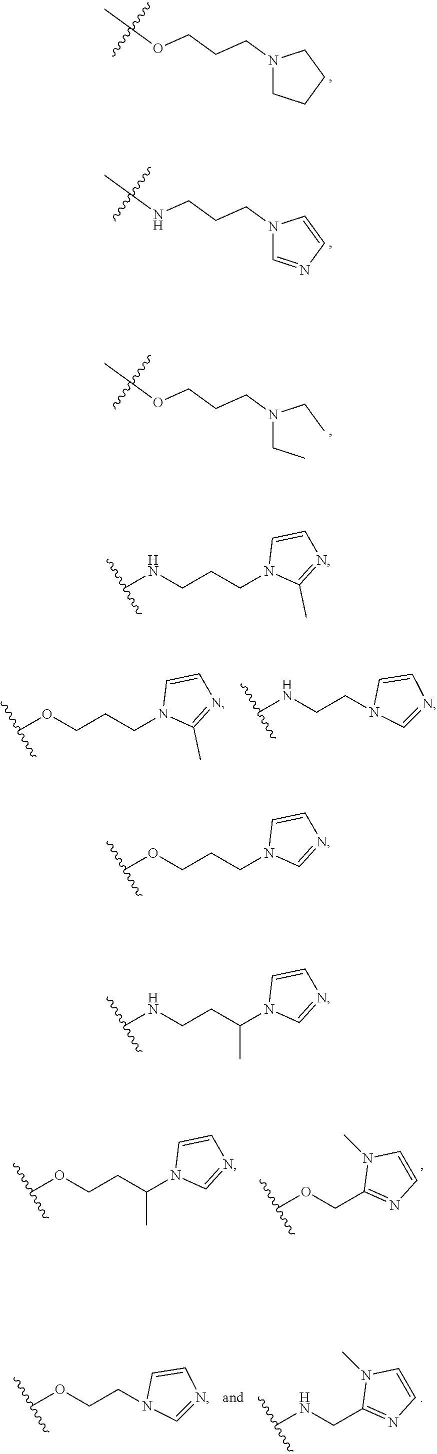

In some embodiments, an LNP including Formula (xxiii) is used to deliver a polyribonucleotide (e.g., a circular polyribonucleotide, a linear polyribonucleotide) composition described herein to cells. In some embodiments, the LNP of Formula (xxiii) is an LNP described by International Patent Publication No. WO2021/113777 (e.g., a lipid of Formula (3) such as a lipid of Table 3 of International Patent Publication No. WO2021/113777).

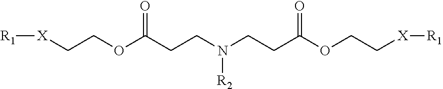

in which
   X is selected from —O—, —S—, or —OC(O)—*, in which * indicates the attachment point to R₁.
R₁ is selected from a group consisting of:

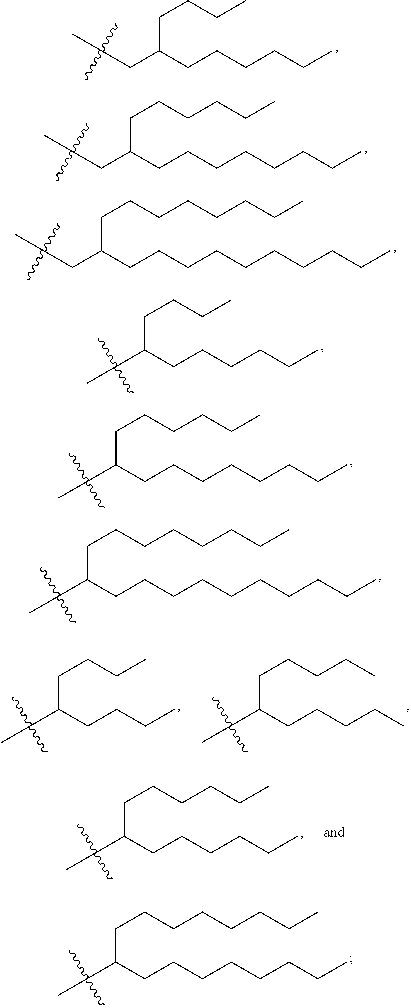

and R₂ is selected from a group consisting of:

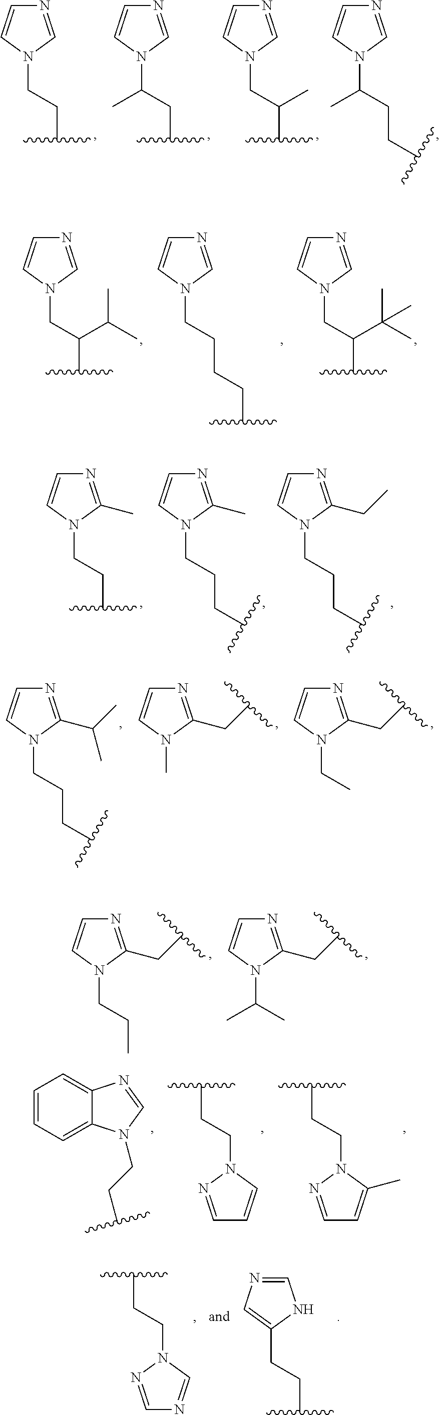

In some embodiments, a composition described herein (e.g., a nucleic acid (e.g., a circular polyribonucleotide, a linear polyribonucleotide) or a protein) is provided in an LNP that includes an ionizable lipid. In some embodiments, the ionizable lipid is heptadecan-9-yl 8-((2-hydroxyethyl) (6-oxo-6-(undecyloxy) hexyl)amino) octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy) butanoyl)oxy)-2-((((3-(diethylamino) propoxy) carbonyl)oxy)methyl) propyl octadeca-9,12-dienoate (LP01), e.g., as synthesized in Example 13 of International Patent Publication No. WO2015/095340 (incorporated by reference herein in its entirety). In some embodiments, the ionizable lipid is Di((Z)-non-2-en-1-yl) 9-((4-dimethylamino)butanoyl)oxy) heptadecanedioate (L319), e.g., as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl) (2-hydroxydodecyl)amino)ethyl) piperazin-1-yl)ethyl) azanediyl)bis(dodecan-2-ol) (C12-200), e.g., as synthesized in Examples 14 and 16 of International Patent Publication No. WO2010/053572 (incorporated by reference herein in its entirety). In some embodiments, the ionizable lipid is Imidazole cholesterol ester (ICE) lipid (3S,10R,13R,17R)-10,13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl) propanoate, e.g., Structure (I) from International Patent Publication No. WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle includes a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol, and polymer conjugated lipids. In some embodiments, the cationic lipid may be an ionizable cationic lipid. An exemplary cationic lipid as disclosed herein may have an effective pKa over 6.0. In some embodiments, a lipid nanoparticle may include a second cationic lipid having a different effective pKa (e.g., greater than the first effective pKa), than the first cationic lipid. A lipid nanoparticle may include between 40 and 60 mol percent of a cationic lipid, a neutral lipid, a steroid, a polymer conjugated lipid, and a therapeutic agent, e.g., a nucleic acid (e.g., RNA (e.g., a circular polyribonucleotide, a linear polyribonucleotide)) described herein, encapsulated within or associated with the lipid nanoparticle. In some embodiments, the nucleic acid is co-formulated with the cationic lipid. The nucleic acid may be adsorbed to the surface of an LNP, e.g., an LNP including a cationic lipid. In some embodiments, the nucleic acid may be encapsulated in an LNP, e.g., an LNP including a cationic lipid. In some embodiments, the lipid nanoparticle may include a targeting moiety, e.g., coated with a targeting agent. In some embodiments, the LNP formulation is biodegradable. In some embodiments, a lipid nanoparticle including one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule.

Exemplary ionizable lipids that can be used in lipid nanoparticle formulations include, without limitation, those listed in Table 1 of International Patent Publication No. WO2019/051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of International Patent Publication No. WO2017/117528; A of US2012/0149894; A of US2015/0057373; A of International Patent Publication No. WO2013/116126; A of US2013/0090372; A of US2013/0274523; A of US2013/0274504; A of US2013/0053572; A of International Patent Publication No. WO2013/016058; A of WO2012/162210; I of US2008/042973; I, II, III, or IV of US2012/01287670; I or II of US2014/0200257; I, II, or III of US2015/0203446; I or Ill of US2015/0005363; I, IA, IB, IC, ID, II, IIA, IIB, IIC, IID, or III-XXIV of US2014/0308304; of US2013/0338210; I, II, III, or IV of International Patent Publication No. WO2009/132131; A of US2012/01011478; I or XXXV of US2012/0027796; XIV or XVII of US2012/0058144; of US2013/0323269; I of US2011/0117125; I, II, or Ill of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or Ill of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of International Patent Publication No. WO2018/081480; 1-5 or 1-8 of International Patent Publication No. WO2020/081938; 18 or 25 of U.S. Pat. No. 9,867,888; A of US2019/0136231; II of International Patent Publication No. WO2020/219876; 1 of US2012/0027803; OF-02 of US2019/0240349; 23 of U.S. Pat. No. 10,086,013; cKK-E12/A6 of Miao et al (2020); C12-200 of International Patent Publication No. WO2010/053572; 7C₁ of Dahlman et al (2017); 304-013 or 503-013 of Whitehead et al; TS-P4C₂ of U.S. Pat. No. 9,708,628; I of International Patent Publication No. WO2020/106946; I of International Patent Publication No. WO2020/106946; and (1), (2), (3), or (4) of International Patent Publication No. WO2021/113777. Exemplary lipids further include a lipid of any one of Tables 1-16 of International Patent Publication No. WO2021/113777.

In some embodiments, the ionizable lipid is MC3 (6Z,9Z,28Z,31Z)-heptatriaconta-6,9,28,31-tetraen-19-yl-4-(dimethylamino)butanoate (DLin-MC3-DMA or MC3), e.g., as described in Example 9 of International Patent Publication No. WO2019/051289A9 (incorporated by reference herein in its entirety). In some embodiments, the ionizable lipid is the lipid ATX-002, e.g., as described in Example 10 of International Patent Publication No. WO2019/051289A9 (incorporated by reference herein in its entirety). In some embodiments, the ionizable lipid is (13Z,16Z)-A,A-dimethyl-3-nonyldocosa-13,16-dien-1-amine (Compound 32), e.g., as described in Example 11 of International Patent Publication No. WO2019/051289A9 (incorporated by reference herein in its entirety). In some embodiments, the ionizable lipid is Compound 6 or Compound 22, e.g., as described in Example 12 of International Patent Publication No. WO2019/051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidylethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in International Patent Publication No. WO2017/099823 or US Patent Publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can include, for example, 0-30% (mol) of the total lipid present in the lipid nanoparticle. In some embodiments, the non-cationic lipid content is 5-20% (mol) or 10-15% (mol) of the total lipid present in the lipid nanoparticle. In some embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not include any phospholipids.

In some embodiments, the lipid nanoparticle can further include a component, such as a sterol, to provide membrane integrity. One exemplary sterol that can be used in the lipid nanoparticle is cholesterol and derivatives thereof. Non-limiting examples of cholesterol derivatives include polar analogues such as 5a-cholestanol, 5β-coprostanol, cholesteryl-(2′-hydroxy)-ethyl ether, cholesteryl′(4′-hydroxy)-butyl ether, and 6-ketocholestanol; non-polar analogues such as 5a-cholestane, cholestenone, 5a-cholestanone, 5β-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can include 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can include a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy) propyl-1-0-(w-methoxy (polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, US2018/0028664, and International Patent Publication No. WO2017/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1′ [8′-(Cholest-5-en-3 [beta]-oxy) carboxamido-3′,6′-dioxaoctanyl]carbamoyl-[omega]-methyl-poly(ethylene glycol), PEG-DMB(3,4-Ditetradecoxylbenzyl-[omega]-methyl-poly(ethylene glycol)ether), and 1,2-dimyristoyl-sn-glycero-3-phosphoethanolamine-N-[methoxy (polyethylene glycol)-2000]. In some embodiments, the PEG-lipid includes PEG-DMG, 1,2-dimyristoyl-sn-glycero-3-phosphoethanolamine-N-[methoxy (polyethylene glycol)-2000]. In some embodiments, the PEG-lipid includes a structure selected from:

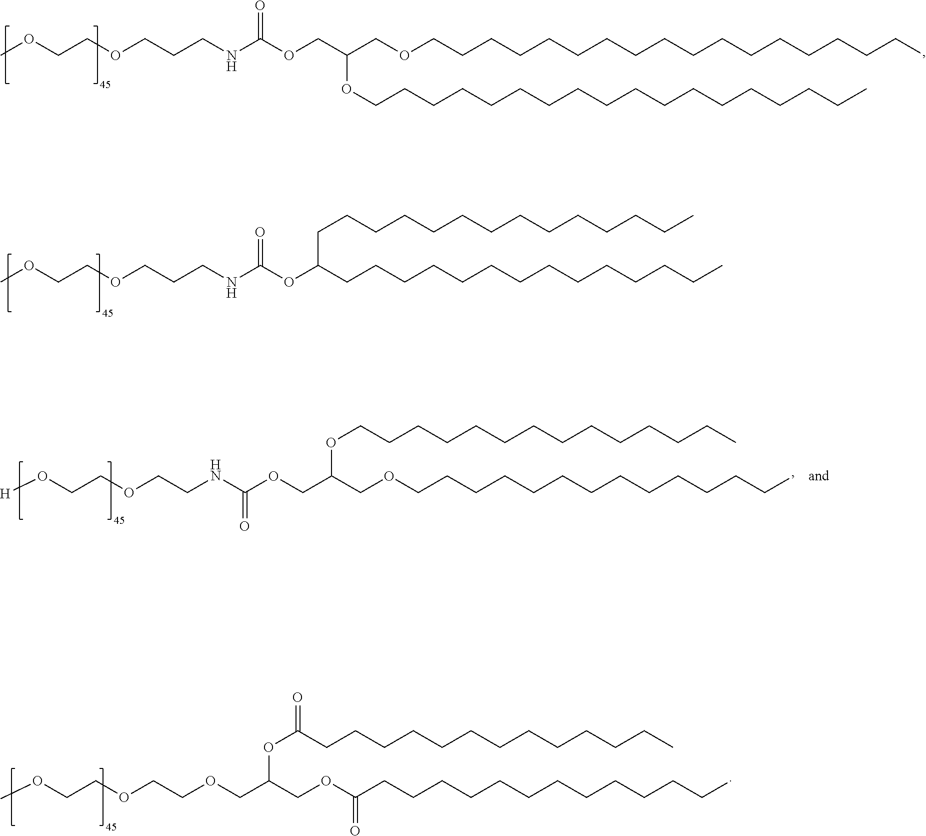

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of International Patent Publication No. WO2019/051289A9, the contents of all of which are incorporated herein by reference in their entirety.

In some embodiments, the PEG or the conjugated lipid can include 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG-conjugated lipid can be varied as needed. For example, the lipid particle can include 30-70% ionizable lipid by mole or by total weight of the composition, 0-60% cholesterol by mole or by total weight of the composition, 0-30% non-cationic lipid by mole or by total weight of the composition and 1-10% conjugated lipid by mole or by total weight of the composition. Preferably, the composition includes 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example including 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation includes ionizable lipid, phospholipid, cholesterol and a PEG-ylated lipid in a molar ratio of 50:10:38.5:1.5. In some other embodiments, the lipid particle formulation includes ionizable lipid, cholesterol and a PEG-ylated lipid in a molar ratio of 60:38.5:1.5.

In some embodiments, the lipid particle includes ionizable lipid, non-cationic lipid (e.g., phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle includes ionizable lipid/non-cationic-lipid/sterol/conjugated lipid at a molar ratio of 50:10:38.5:1.5.

In an aspect, the disclosure provides a lipid nanoparticle formulation including phospholipids, lecithin, phosphatidylcholine and phosphatidylethanolamine.

In some embodiments, one or more additional compounds can also be included. Those compounds can be administered separately, or the additional compounds can be included in the lipid nanoparticles of the disclosure. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the LNPs include biodegradable, ionizable lipids. In some embodiments, the LNPs include (9Z,12Z)-3-((4,4-bis(octyloxy) butanoyl)oxy)-2-((((3-(diethylamino) propoxy) carbonyl)oxy)methyl) propyl octadeca-9,12-dienoate, also called 3-((4,4-bis(octyloxy) butanoyl)oxy)-2-((((3-(diethylamino) propoxy) carbonyl)oxy)methyl) propyl (9Z,12Z)-octadeca-9,12-dienoate) or another ionizable lipid. See, e.g., lipids of International Patent Publication Nos. WO2019/067992, WO/2017/173054, WO2015/095340, and WO2014/136086, as well as references provided therein. In some embodiments, the term cationic and ionizable in the context of LNP lipids is interchangeable, e.g., in which ionizable lipids are cationic depending on the pH.

In some embodiments, the average LNP diameter of the LNP formulation may be between 10 s of nm and 100 s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

An LNP can, in some instances, be relatively homogenous. A polydispersity index may be used to indicate the homogeneity of an LNP, e.g., the particle size distribution of the lipid nanoparticles. A small (e.g., less than 0.3) polydispersity index generally indicates a narrow particle size distribution. An LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of an LNP may be from about 0.10 to about 0.20.

The zeta potential of an LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of an LNP may be from about-10 mV to about +20 mV, from about-10 mV to about +15 mV, from about-10 mV to about +10 mV, from about-10 mV to about +5 mV, from about-10 mV to about 0 mV, from about-10 mV to about-5 mV, from about-5 mV to about +20 mV, from about-5 mV to about +15 mV, from about-5 mV to about +10 mV, from about-5 mV to about +5 mV, from about-5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with an LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

An LNP may optionally include one or more coatings. In some embodiments, an LNP may be formulated in a capsule, film, or tablet having a coating. A capsule, film, or tablet including a composition described herein may have any useful size, tensile strength, hardness or density.

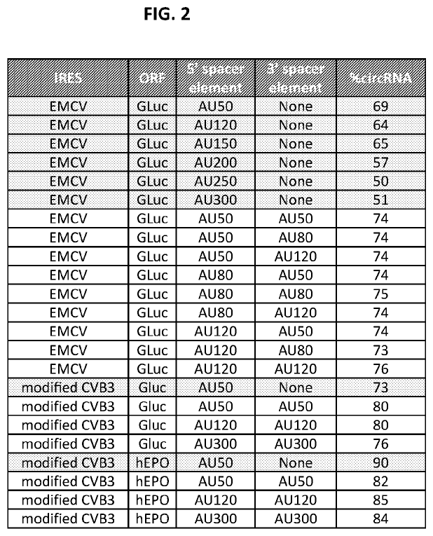

Additional exemplary lipids, formulations, methods, and characterization of LNPs are taught by International Patent Publication Nos. WO2020/061457, WO2021/113777, WO2021/226597, and WO2022/261490, each of which is incorporated herein by reference in its entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al).

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in International Patent Publication No. WO2019067992 and International Patent Publication No. WO2019/067910, both incorporated by reference, and are useful for delivery of circular polyribonucleotides and linear polyribonucleotides described herein.

Additional specific LNP formulations useful for delivery of nucleic acids (e.g., circular polyribonucleotides, linear polyribonucleotides) are described in U.S. Pat. Nos. 8,158,601 and 8,168,775, both incorporated by reference, which include formulations used in patisiran, sold under the name ONPATTRO.

Exemplary dosing of polyribonucleotide (e.g., a circular polyribonucleotide, a linear polyribonucleotide) LNP may include about 0.1, 0.25, 0.3, 0.5, 1, 2, 3, 4, 5, 6, 8, 10, or 100 mg/kg (RNA). Exemplary dosing of AAV including a polyribonucleotide (e.g., a circular polyribonucleotide, a linear polyribonucleotide) may include an MOI of about 10¹¹, 10¹², 10¹³, and 10¹⁴ vg/kg.

### Kits

In some aspects, the disclosure provides a kit. In some embodiments, the kit includes (a) a circular polyribonucleotide described herein or a composition (e.g., pharmaceutical or agricultural composition) described herein, and optionally (b) informational material. The informational material may be descriptive, instructional, marketing or other material that relates to the methods described herein and/or the use of the composition or circular polyribonucleotide for the methods described herein. The composition or circular polyribonucleotide may include material for a single administration (e.g., single dosage form), or may include material for multiple administrations (e.g., a “multidose” kit).

The informational material of the kits is not limited in its form. In one embodiment, the informational material may include information about production of a pharmaceutical composition, a pharmaceutical drug substance, or a pharmaceutical drug product, molecular weight of the pharmaceutical composition, the pharmaceutical drug substance, or the pharmaceutical drug product, concentration, date of expiration, batch or production site information, and so forth. In one embodiment, the informational material relates to methods for administering a dosage form of the pharmaceutical composition. In one embodiment, the informational material relates to methods for administering a dosage form of the circular polyribonucleotide.

In some embodiments, in addition to a dosage form of the pharmaceutical composition and circular polyribonucleotide described herein, the kit may include other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients may be included in the kit, but in different compositions or containers than a pharmaceutical composition or circular polyribonucleotide described herein. In such embodiments, the kit may include instructions for admixing a pharmaceutical composition or nucleic acid molecule (e.g., a circular polyribonucleotide) described herein and the other ingredients, or for using a pharmaceutical composition or nucleic acid molecule (e.g., a circular polyribonucleotide) described herein together with the other ingredients.

In some embodiments, the components of the kit are stored under inert conditions (e.g., under Nitrogen or another inert gas such as Argon). In some embodiments, the components of the kit are stored under anhydrous conditions (e.g., with a desiccant). In some embodiments, the components are stored in a light blocking container such as an amber vial.

A dosage form of a composition (e.g., pharmaceutical or agricultural composition) or polyribonucleotide (e.g., a circular polyribonucleotide) described herein may be provided in any form, e.g., liquid, dried or lyophilized form. It is preferred that a composition (e.g., pharmaceutical or agricultural composition) or polyribonucleotide (e.g., a circular polyribonucleotide) described herein be substantially pure and/or sterile. When a composition (e.g., pharmaceutical or agricultural composition) or polyribonucleotide (e.g., a circular polyribonucleotide) described herein is provided in a liquid solution, the liquid solution preferably is an aqueous solution, with a sterile aqueous solution being preferred. When a composition (e.g., pharmaceutical or agricultural composition) or polyribonucleotide (e.g., a circular polyribonucleotide) described herein is provided as a dried form, reconstitution generally is by the addition of a suitable solvent. The solvent, e.g., sterile water or buffer, can optionally be provided in the kit.

The kit may include one or more containers for the composition containing a dosage form described herein. In some embodiments, the kit contains separate containers, dividers or compartments for the composition and informational material. For example, the composition (e.g., pharmaceutical or agricultural composition) or polyribonucleotide (e.g., a circular polyribonucleotide) may be contained in a bottle, vial, or syringe, and the informational material may be contained in a plastic sleeve or packet. In other embodiments, the separate elements of the kit are contained within a single, undivided container. For example, the dosage form of a composition (e.g., pharmaceutical or agricultural composition) or polyribonucleotide (e.g., a circular polyribonucleotide) described herein is contained in a bottle, vial or syringe that has attached thereto the informational material in the form of a label. In some embodiments, the kit includes a plurality (e.g., a pack) of individual containers, each containing one or more unit dosage forms of a composition (e.g., pharmaceutical or agricultural composition) or polyribonucleotide (e.g., a circular polyribonucleotide) described herein. For example, the kit includes a plurality of syringes, ampules, foil packets, or blister packs, each containing a single unit dose of a dosage form described herein.

The containers of the kits can be airtight, waterproof (e.g., impermeable to changes in moisture or evaporation), and/or light-tight.

The kit optionally includes a device suitable for use of the dosage form, e.g., a syringe, pipette, forceps, measured spoon, swab (e.g., a cotton swab or wooden swab), or any such device.

The kits of the invention may include dosage forms of varying strengths to provide a subject with doses suitable for one or more of the initiation phase regimens, induction phase regimens, or maintenance phase regimens described herein. Alternatively, the kit may include a scored tablet to allow the user to administer divided doses, as needed.

### EXAMPLES

The following examples are included for illustrative purposes only and are not intended to limit the scope of the invention. Each of the DNA sequences described herein include the RNA equivalent sequence, as would be understood by one skilled in the art. Likewise, all RNA sequences described herein include the DNA equivalent sequence, as would be understood by one skilled in the art.

### Example 1: Effect of Dual Spacer Design and Spacer Length on Circularization Efficiency

This example demonstrates the effect of a dual spacer design and spacer length on circularization efficiency of constructs including spacer elements.

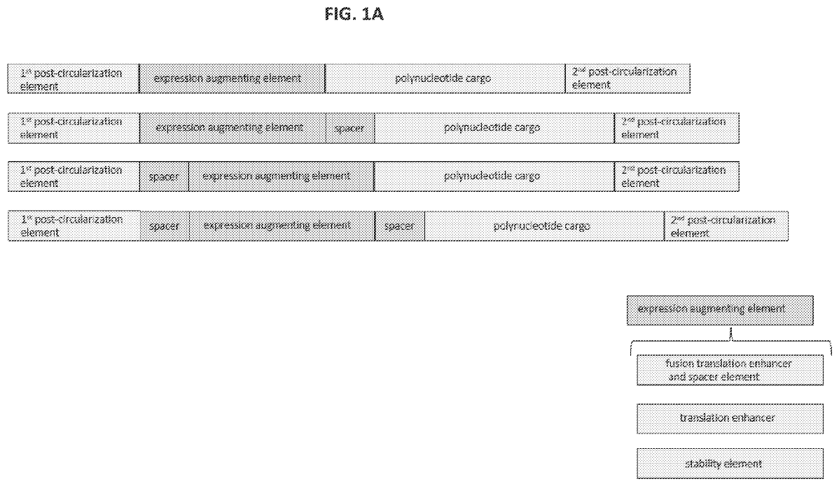

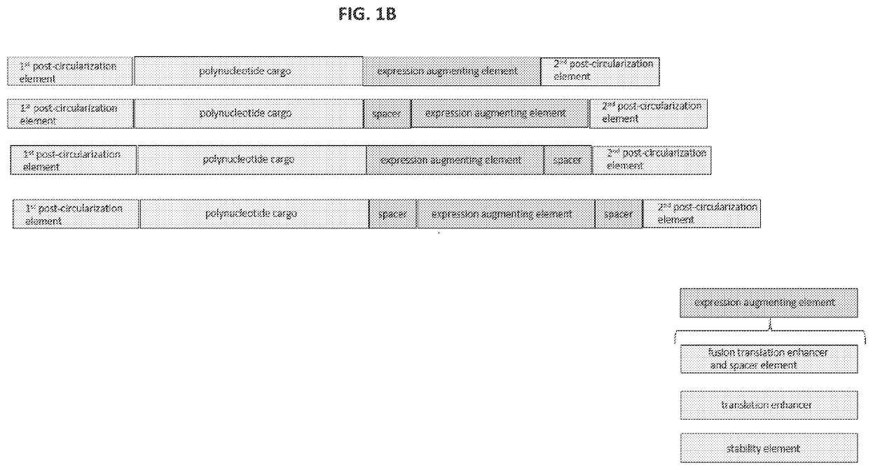

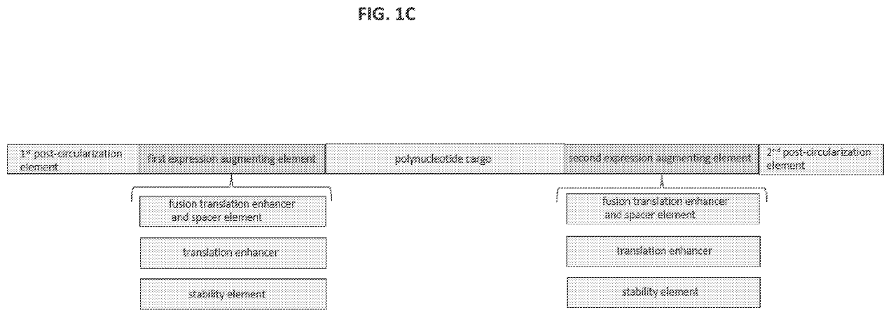

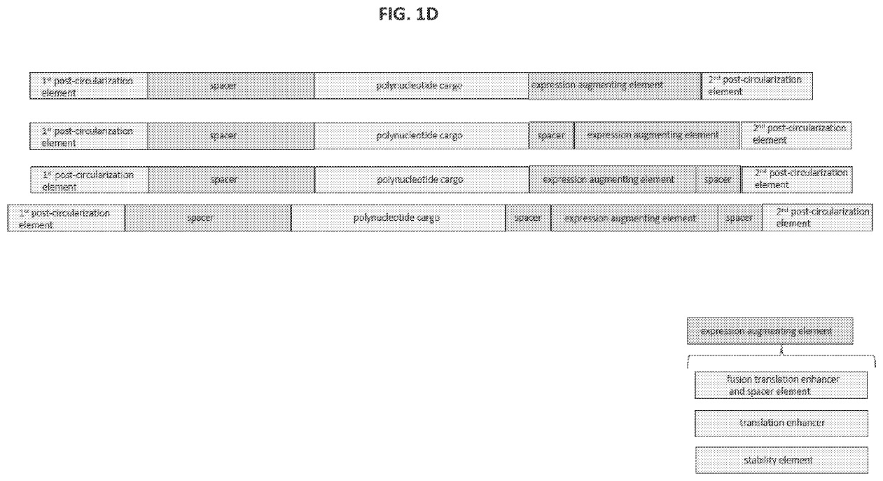

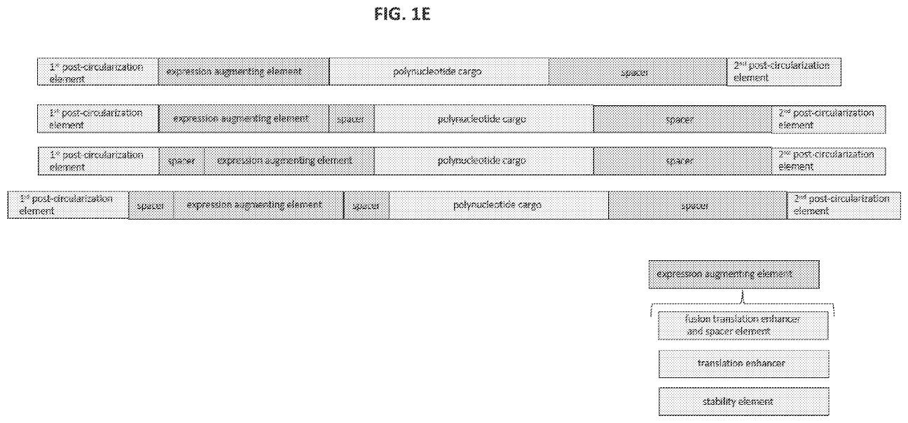

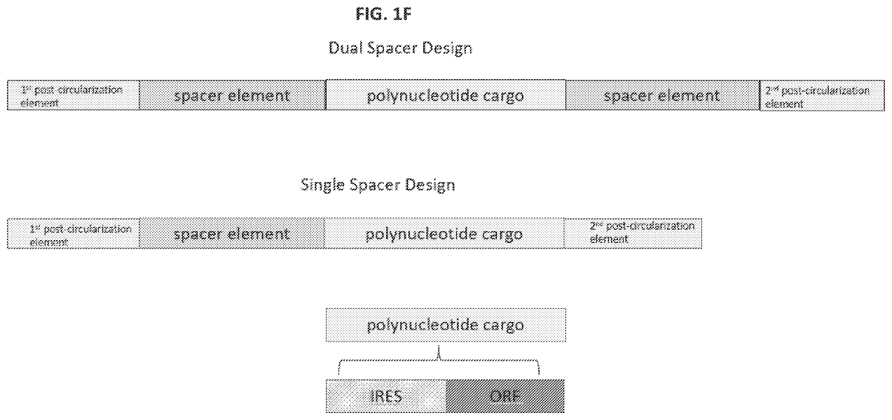

DNA constructs were designed to include an internal ribosome entry site (IRES), an open reading frame (ORF), and a 5′ spacer element (“single spacer design”) or both a 5′ spacer element and a 3′ spacer element (“dual spacer design”). An exemplary schematic of the single spacer design and the dual spacer design are provided in FIG. 1F. In this example, the DNA constructs included an EMCV or modified CVB3 nucleotide sequence as the IRES, a Gaussia luciferase (Gluc) or human erythropoietin (hEPO) nucleotide sequence as the ORF, and polyAT sequences as the spacer element(s). The poly AT spacer elements were designed between 50 and 300 nucleotides in length.

Circular RNAs were generated by self-splicing using a method described herein. Unmodified linear RNA was synthesized by in-vitro transcription using T7 RNA polymerase from a DNA template including the motifs listed above in the presence of 7.5 mM of NTP. Template DNA was removed by treating with DNase. Synthesized linear RNA was purified with an RNA clean up kit (New England Biolabs, T2050). Circularization efficiency was determined by analytical AEX-HPLC and is presented as % circRNA.

In a single spacer design, circularization efficiency shows a negative correlation with spacer element length in the absence of a 3′ spacer element (FIG. 2, as indicated by % circRNA). Circularization efficiency increases and stabilizes with the addition of a 3′ spacer element. In a dual spacer design, circularization efficiency does not decrease with increasing spacer element length and exceeds that of any single 5′ spacer design with a spacer element of the same length (FIG. 2, as indicated by % circRNA).

### Example 2: Effect of Spacers and Translation Enhancers on Circularization Efficiency

This example demonstrates the effect of a dual design on circularization efficiency of constructs including spacer elements and translation enhancer motifs.

DNA Constructs were Designed and Included, from 5′ to 3′:
(i) 5′ translation enhancer, IRES, and ORF (“single design); and
(ii) 5′ spacer element, IRES, ORF, and 3′ translation enhancer (“dual design).

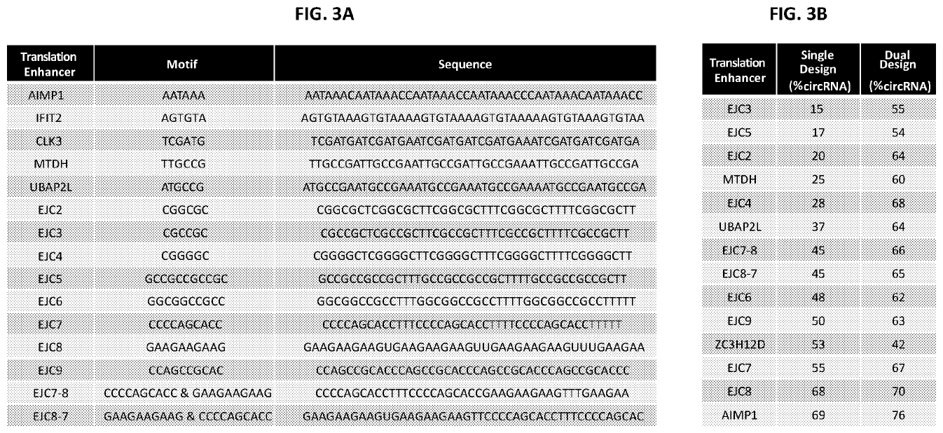

In this example, the DNA constructs included an EMCV IRES and a Gluc nucleotide sequence as the ORF. Translation enhancer elements were designed with repeats of translation enhancing motifs gaped by 1-5 “A”, “C” or “T” nucleotides up to 50 nt in length (FIG. 3A).

For the single design, the 5′ translation enhancer is a translation enhancer sequence provided in FIG. 3A. For the dual design, the 5′ spacer element is a polyAT sequence of 50 nucleotides in length and the 3′ translation enhancer is a translation enhancer sequence provided in FIG. 3A.

Circular RNAs were generated by self-splicing using a method described herein. Unmodified linear RNA was synthesized by in-vitro transcription using T7 RNA polymerase from a DNA template including the motifs listed above in the presence of 7.5 mM of NTP. Template DNA was removed by treating with DNase. Synthesized linear RNA was purified with an RNA clean up kit (New England Biolabs, T2050). Circularization efficiency was determined by analytical AEX-HPLC and is presented as % circRNA.

In a dual design, circularization efficiency is consistently higher than that of a single spacer design containing the same translation enhancer element sequence (FIG. 3B).

### Example 3: Effect of Spacer Length on Circular RNA Expression in a Single Spacer Design

This example demonstrates the effect of spacer length and sequences on expression of ORFs from circular RNAs in mammalian cells.

In this example, DNA constructs were designed to include an IRES, an ORF, and a 5′ spacer element in a single spacer design. The DNA constructs included an EMCV IRES or a modified CVB3 IRES, a Gluc or hEPO nucleotide sequence as the ORF, and a polyAT, polyAG or polyAC sequence as the 5′ spacer element in a single spacer design. The spacer elements were designed to be 50, 80, or 120 nucleotides in length. A construct designed to include polyA50 as a 5′ UTR was used as a control.

In this example, the circular RNAs were generated by self-splicing using a method described herein. Unmodified RNA was synthesized by in vitro transcription using T7 RNA polymerase from a DNA template including the motifs listed above in the presence of 7.5 mM of NTP. Circularization occurs during transcription, so no additional reaction is required. Template DNA was removed by treating with DNase. Synthesized RNA was purified with an RNA clean up kit (New England Biolabs, T2050). Circular RNA encoding Gluc or hEPO was purified by reversed phase column chromatography or linear RNA pull-down (LP) to enrich circular RNA.

To pull down linear byproducts that contain intronic sequences, six different oligomers against intronic region were designed, four for 3′ half-intron, and two for 5′ half-intron. Self-spliced RNA (1 nmol) was mixed with 2 nmol of each oligo in the presence of 1× binding buffer (150 mM NaCl, 15 mM sodium citrate, 0.5 mM EDTA) in 2.5 ml total (final RNA concentration was 400 nM and oligomer concentration was 800 nM per each oligomer). The RNA-oligomer mixtures were incubated for 30 minutes at room temperature. For column purification, 500 μl of bead was pre-packed in an empty column and RNA-oligomer mixture was placed on the packed resin. The RNA-oligomer mixture was passed through the resin by gravity and flow through was collected. The collected flow through was mixed with additional oligomers (final 800 nM), then passed through freshly pre-packed resin. The flow through was collected and the concentration of the first and second round of flow through was measured by Qubit assay.

Circular RNAs (0.1 picomoles) were transfected into Hela cells (10,000 cells per well in a 96 well plate in serum-free media) using LIPOFECTAMINE® MessengerMAX transfection reagent (Invitrogen) according to manufacturer's instructions. MessengerMax alone was used as a control (No RNA). Cell culture media was harvested and replaced with fresh media at 24 hour and 48 hour timepoints to measure Gluc activity.

To measure expression of circular RNA encoding Gluc (Gluc activity), 10 μl of harvested cell media was transferred to a white 96 well plate, and a bioluminescent reporter assay system was used according to the manufacturer's instruction (Pierce Gaussia luciferase Flash Assay Kit, 16158, Thermo Scientific). The plate was read in a luminometer instrument (Promega).

To measure expression of circular RNA encoding hEPO, supernatant was collected at 24 hour and 48 hour. Expression was measured using a hEPO-specific ELISA.

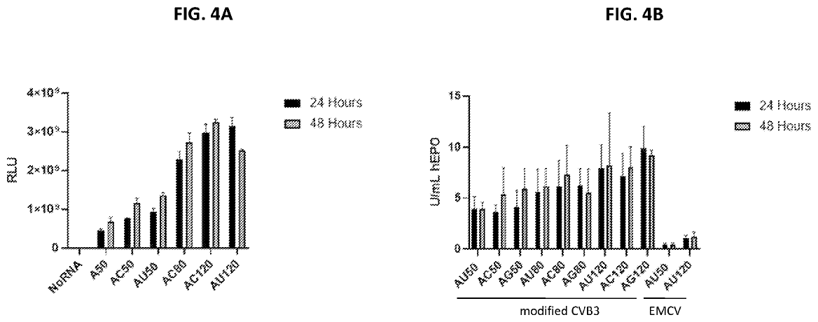

Increased spacer length enhanced expression of Gluc from a EMCV IRES (FIG. 4A) and hEPO from a EMCV or modified CVB3 IRES (FIG. 4B) in a circular RNA construct. The polyAU, polyAG or polyAC 5′ spacer elements yielded higher expression than the polyA sequence (control). Similar expression levels are observed for polyAU, polyAG or polyAC 5′ spacer elements of the same length. 5′ spacer element with longer nucleotide sequences express better than 5′ spacer elements with shorter nucleotide sequences regardless of their nucleotide composition.

### Example 4: Effect of Spacer Length on Circular RNA Expression

This example demonstrates the effect of spacer length in both single and dual spacer designs on expression of ORFs from circular RNAs in mammalian cells.

In this example, DNA constructs were designed to include an IRES, an ORF, and a 5′ spacer element (“single spacer design”), or both a 5′ spacer element and a 3′ spacer element (“dual spacer element design”). DNA constructs included an EMCV IRES, a Gluc nucleotide sequence as the ORF, and polyAT sequence(s) as the spacer elements. Spacer elements were designed between 50 and 300 nucleotides in length.

In this example, the circular RNAs were generated by self-splicing using a method described herein. Unmodified linear RNA was synthesized by in vitro transcription using T7 RNA polymerase from a DNA template including the motifs listed above in the presence of 7.5 mM of NTP. Template DNA was removed by treating with DNase. Synthesized linear RNA was purified with an RNA clean up kit (New England Biolabs, T2050). Circular RNA encoding Gluc was purified by LP as described in Example 3.

Circular RNAs (0.025 picomoles) were transfected into Hela cells (10,000 cells per well in a 96 well plate in serum-free media) using LIPOFECTAMINE® MessengerMAX transfection reagent (Invitrogen) according to manufacturer's instructions. MessengerMax alone was used as a control (No RNA). Cell culture media was harvested at 24-hour timepoint to measure Gluc activity.

To measure expression of circular RNA encoding Gluc (Gluc activity), 10 μl of harvested cell media was transferred to a white 96 well plate, and a bioluminescent reporter assay system was used according to the manufacturer's instruction (Pierce Gaussia luciferase Flash Assay Kit, 16158, Thermo Scientific). The plate was read in a luminometer instrument (Promega).

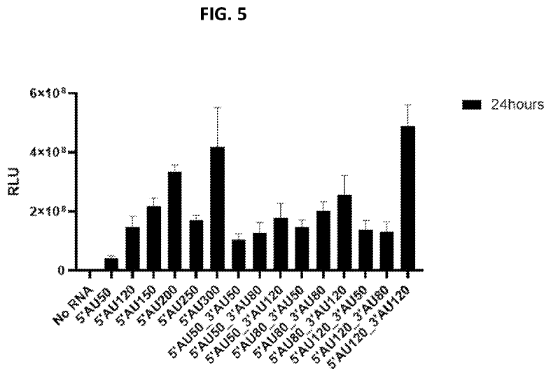

Increased spacer lengths resulted in enhanced expression of Gluc from circular RNA with single and dual spacer designs (FIG. 5). In the absence of a 3′ spacer element, increased 5′ spacer element length up to 300 nt enhanced expression of Gluc from circular RNA. In a dual spacer design, increasing 3′ spacer element length with a constant 5′ spacer element length enhanced expression of Gluc from circular RNA.

### Example 5: Effect of Spacer Length in a Dual Spacer Design on Circular RNA Expression

This example demonstrates the effect of a dual spacer design with identical lengths and sequences on circularization efficiency and expression of ORFs from circular RNAs in mammalian cells.

In this example, DNA constructs were designed to include an IRES, an ORF, and a 5′ spacer element (“single spacer design”), or both a 5′ spacer element and a 3′ spacer element (“dual spacer design”). DNA constructs included an EMCV IRES, a Gluc nucleotide sequence as the ORF, and polyAT sequence(s) as the spacer elements, as shown in Table 21. Spacer elements were designed between 50 and 300 nucleotides in length.

TABLE 21
Components of constructs

| 5′ spacer | | | 3′ spacer |
|---|---|---|---|
| element | IRES | ORF | element |
| AT50 | CVB3 | Gluc or hEPO | None |
| AT50 | CVB3 | Gluc or hEPO | AT50 |
| AT120 | CVB3 | Gluc or hEPO | AT120 |
| AT300 | CVB3 | Gluc or hEPO | AT300 |

In this example, the circular RNAs were generated by self-splicing using a method described herein. Unmodified linear RNA was synthesized by in vitro transcription using T7 RNA polymerase from a DNA template including the motifs listed above in the presence of 7.5 mM of NTP. Template DNA was removed by treating with DNase. Synthesized linear RNA was purified with an RNA clean up kit (New England Biolabs, T2050).

Circular RNAs (0.025 picomoles) were transfected into Hela cells (10,000 cells per well in a 96 well plate in serum-free media) using LIPOFECTAMINE® MessengerMAX transfection reagent (Invitrogen) according to manufacturer's instructions. MessengerMax alone was used as a control (No RNA). Cell culture media was harvested and replaced with fresh media at 4-hour, 24-hour and 48-hour timepoints to measure Gluc activity or hEPO protein levels.

To measure expression of circular RNA encoding Gluc (Gluc activity), 10 μl of harvested cell media was transferred to a white 96 well plate, and a bioluminescent reporter assay system was used according to the manufacturer's instruction (Pierce Gaussia luciferase Flash Assay Kit, 16158, Thermo Scientific). The plate was read in a luminometer instrument (Promega).

Expression of circular RNA encoding hEPO was measured using a hEPO-specific ELISA.

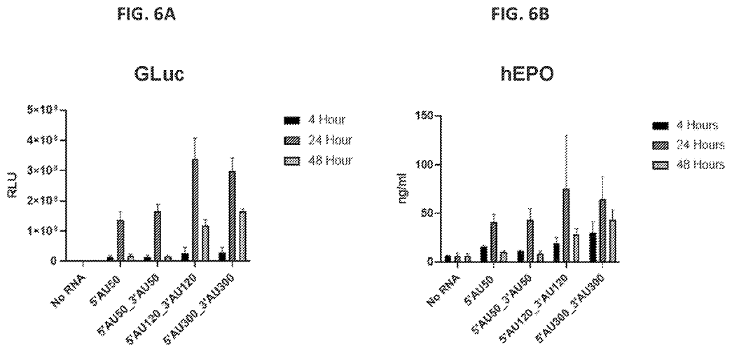

In a dual spacer design increasing spacer element length up to 120 nt enhanced expression from circular RNA (FIGS. 6A and 6B). Increasing spacer element length to 300 nt did not decrease Gluc or hEPO expression.

### Example 6: Effect of Spacer Elements and Translation Enhancers on Circular RNA Expression

This example demonstrates the effect of sequences containing translation enhancers and spacer elements on expression of ORFs from circular RNAs in mammalian cells.

DNA constructs were designed to include a 5′ spacer element, an IRES, an ORF, and a 3′ translation enhancer (“dual design”). In this example, the DNA constructs included an EMCV IRES, a Gluc or hEPO nucleotide sequence as the ORF, a polyAT sequence of 50 nucleotides in length as the 5′ spacer element, and a translation enhancer sequence provided in FIG. 3A as the 3′ translation enhancer. A DNA construct designed to include a polyAT sequence of 50 nucleotides in length as both the 5′ and 3′ spacer elements was used as control.

Circular RNAs were generated by self-splicing using a method described herein. Unmodified linear RNA was synthesized by in vitro transcription using T7 RNA polymerase from a DNA template including the motifs listed above in the presence of 7.5 mM of NTP. Template DNA was removed by treating with DNase. Synthesized linear RNA was purified with an RNA clean up kit (New England Biolabs, T2050). Circular RNA encoding Gluc was purified by LP as described in Example 3.

Circular RNAs (0.1 picomoles) were transfected into A549 cells (10,000 cells per well in a 96 well plate in serum-free media) using LIPOFECTAMINE® MessengerMAX transfection reagent (Invitrogen) according to manufacturer's instructions. MessengerMax alone was used as a control (No RNA). Cell culture media was harvested at 24 hours to measure Gluc activity or hEPO protein levels.

To measure expression of circular RNA encoding Gluc (Gluc activity), 10 μl of harvested cell media was transferred to a white 96 well plate, and a bioluminescent reporter assay system was used according to the manufacturer's instruction (Pierce Gaussia luciferase Flash Assay Kit, 16158, Thermo Scientific). The plate was read in a luminometer instrument (Promega).

Expression of circular RNA encoding hEPO was measured using a hEPO-specific ELISA.

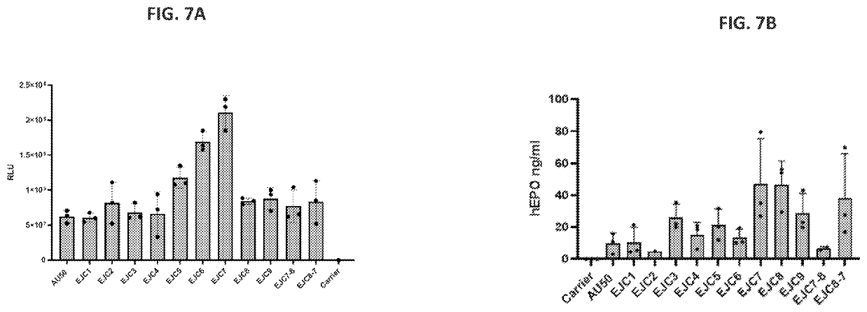

For Gluc, FIG. 7A shows that constructs including the translation enhancer sequences EJC5, EJC6 and EJC7 yielded highest Gluc activity. For hEPO, FIG. 7B shows that constructs including the translation enhancer sequences EJC7, EJC8 and EJC8-7 yielded the highest hEPO levels.

### Example 7: Effect of Translation Enhancers on Circular RNA Expression In Vitro

This example demonstrates the effect of translation enhancers on expression of ORFs from circular RNAs in mammalian cells.

DNA constructs were designed to include, from 5′ to 3′: an IRES, an ORF, and a translation enhancer. In this example, the DNA constructs included an EMCV IRES, an hEPO nucleotide sequence as the ORF, and Thin Paspalum Asymptomatic Virus (TPAV) 3′ UTR as the translation enhancer. A construct without the translation enhancer was used as a control.

In this example, the circular RNAs were generated by self-splicing using a method described herein. Unmodified linear RNA was synthesized by in vitro transcription using T7 RNA polymerase from a DNA template including the motifs listed above in the presence of 7.5 mM of NTP. Template DNA was removed by treating with DNase. Synthesized linear RNA was purified with an RNA clean up kit (New England Biolabs, T2050) and further purified by reversed phase column chromatography.

Circular RNAs (0.025 picomoles per well) were transfected into HEK cells (10,000 cells per well in a 96 well plate in DMEM with 10% FBS) or Hela cells (10,000 cells per well in a 96 well plate in DMEM with 10% FBS) using LIPOFECTAMINE® MessengerMAX transfection reagent (Invitrogen) according to manufacturer's instructions.

To measure expression of circular RNA encoding hEPO, supernatant was collected at 24 hour and 48-hour post transfection. Expression was measured using a hEPO-specific ELISA.

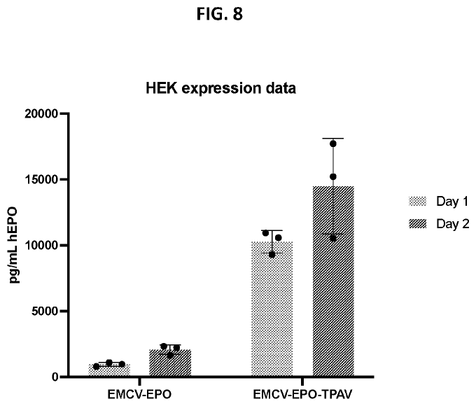

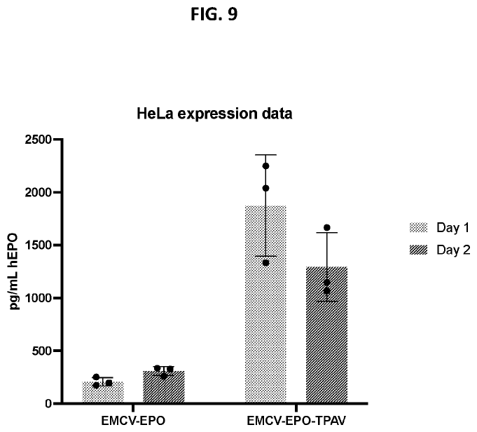

In both cell lines, circular RNAs including the TPAV element expressed 5-10-fold higher than the control, at both 24 and 48 hours post transfection (FIGS. 8 and 9).

### Example 8: Effect of Translation Enhancers on Circular RNA Expression in Mouse Model

This example demonstrates the effect of translation enhancers on expression of ORFs from circular RNAs in mice.

In this example, DNA constructs were designed as described in Example 7. Circular RNAs were produced as described in Example 7.

Circular RNAs were formulated with lipid nanoparticles and administered to mice at 1 mg/kg. Blood samples were collected at 6 hours post dosing. Expression of circular RNA encoding hEPO was measured using a hEPO-specific ELISA.

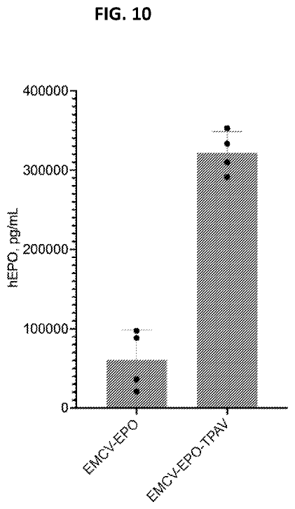

At 6 hours, circular RNAs including the TPAV element expressed 5-fold higher than the control (FIG. 10).

### Example 9: Effect of Spacers and Translation Enhancers on Circular RNA Expression In Vitro

This example demonstrates the effect of spacer elements and translation enhancers on expression of ORFs from circular RNAs in mammalian cells.

The Following DNA Constructs were Designed and Included, from 5′ to 3′:
(i) 5′ spacer element, IRES, ORF, and 3′ spacer element;
(ii) 5′ spacer element, IRES, ORF, translation enhancer, and 3′ spacer element;
(iii) 5′ spacer element, IRES, ORF, and 3′ translation enhancer; and
(iv) 5′ spacer element, translation enhancer, IRES, ORF, and 3′ spacer element.
   In this example, the DNA constructs included a modified CVB3 IRES or EMCV IRES, an hEPO nucleotide sequence as the ORF, a polyAT sequence of 120 nucleotides in length as the spacer element, and TPAV 3′ UTR as the translational enhancer. A construct without the translation enhancer was used as a control.

In this example, the circular RNAs were generated by self-splicing using a method described herein. Unmodified linear RNA was synthesized by in vitro transcription using T7 RNA polymerase from a DNA template including the motifs listed above in the presence of 7.5 mM of NTP. Template DNA was removed by treating with DNase. Synthesized linear RNA was purified with an RNA clean up kit (New England Biolabs, T2050). Circular RNA was purified using a method described herein.

Circular RNAs (0.025 picomoles) were transfected into A549 cells (20,000 cells per well in a 96 well plate in DMEM with 10% FBS) using LIPOFECTAMINE® MessengerMAX transfection reagent (Invitrogen) according to manufacturer's instructions.

To measure expression of circular RNA encoding hEPO, supernatant was collected at 24 hours post transfection. Expression was measured using a hEPO-specific ELISA.

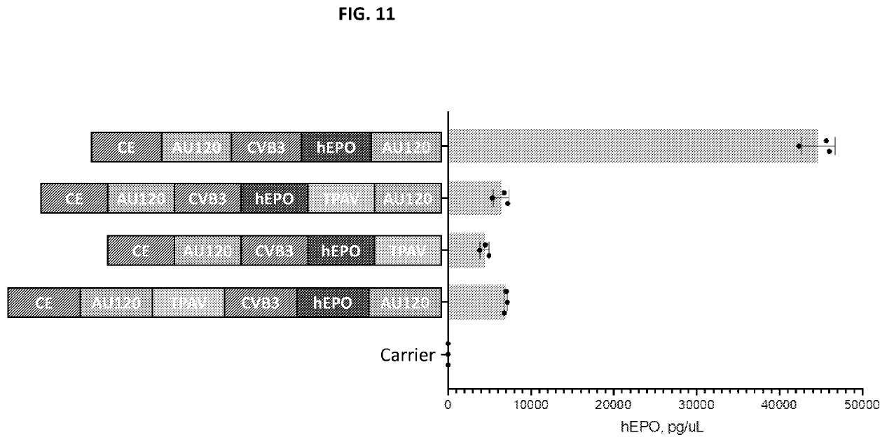

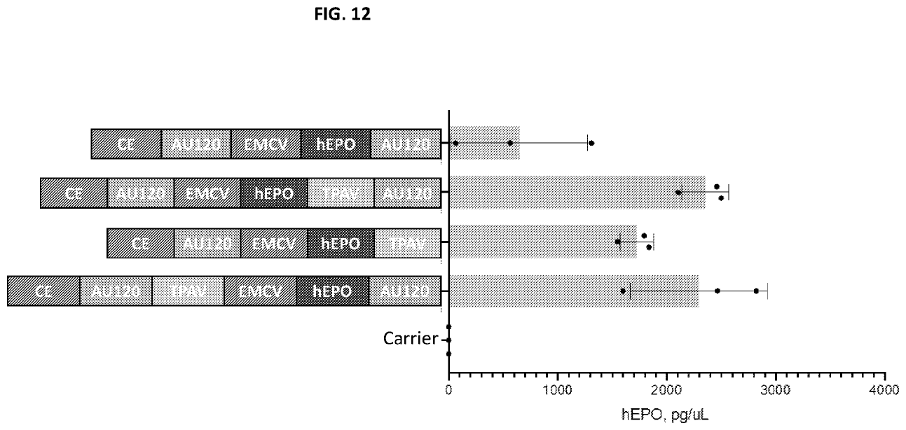

FIG. 11 shows that expression driven by a CVB3 IRES was inhibited by the presence of the TPAV element in any position of the construct. FIG. 12 shows that expression driven by an EMCV IRES was increased by the presence of the TPAV element placed in any position of the construct.

### Example 10: Effect of Translation Enhancers on Circular RNA Expression In Vitro

This example demonstrates the effect of multiple copies of translation enhancers on expression of multiple ORFs from circular RNAs in mammalian cells.

Circular RNAs were designed to include multiple IRESes and multiple ORFs. The following DNA constructs were designed and included, from 5′ to 3′:
(i) spacer element, translation enhancer, IRES, ORF1, translation enhancer, spacer element, IRES, ORF2, translation enhancer, and spacer element;
(ii) spacer element, translation enhancer, IRES, ORF1, translation enhancer, spacer element, IRES, ORF2, spacer element;
(iii) spacer element, IRES, ORF1, spacer element, IRES, ORF2, translation enhancer, and spacer element; and
(iv) spacer element, IRES, ORF1, spacer element, IRES, ORF2, and spacer element.

In this example, the DNA constructs included a modified CVB3 IRES, an hEPO nucleotide sequence as ORF1, a SARS-COV-2 RBD nucleotide sequence as ORF2, a polyAT sequence of 120 nucleotides in length as the spacer element(s), and TPAV 3′ UTR as the translation enhancer. A construct without the translation enhancer was used as a control.

In this example, the circular RNAs were generated by self-splicing using a method described herein. Unmodified linear RNA was synthesized by in vitro transcription using T7 RNA polymerase from a DNA template including the motifs listed above in the presence of 7.5 mM of NTP. Template DNA was removed by treating with DNase. Synthesized linear RNA was purified with an RNA clean up kit (New England Biolabs, T2050). Circular RNA was purified by a method described herein.

Circular RNAs (0.025 picomoles) were transfected into A549 cells (20,000 cells per well in a 96 well plate in serum-free media) using LIPOFECTAMINE® MessengerMAX transfection reagent (Invitrogen) according to manufacturer's instructions.

To measure expression hEPO from circular RNA, supernatant was collected at 24 hour. Expression was measured using a hEPO-specific ELISA.

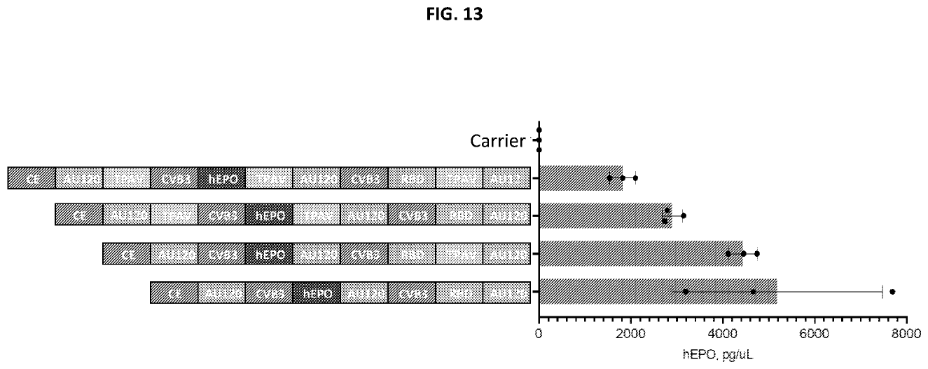

FIG. 13 shows that the expression of hEPO was decreased proportionally with increasing TPAV elements. The result confirms the results of Example 8 that the TPAV element increased expression from EMCV-containing constructs (FIG. 12) and inhibited expression from CVB3-containing constructs (FIG. 11).

### Example 11: Effect of Translation Enhancer on Circular RNA Expression In Vitro

This example demonstrates the effect of translation enhancers on expression of ORFs from circular RNAs in mammalian cells.

For Experiment 1 and Experiment 2, DNA constructs included, from 5′ to 3′: 5′ spacer element, IRES, ORF, and translation enhancer.

For Experiment 3, DNA constructs included, from 5. to 3′: 5′ spacer element, IRES, ORF, translation enhancer, and 3′ spacer element.

For Experiment 4, DNA constructs included, from 5′ to 3′: translation enhancer, translation enhancer, 5′ spacer element, IRES, and ORF.

### Experiment 1: EGFPd2

The DNA constructs included a modified CVB3 IRES, an EGFPd2 nucleotide sequence as the ORF, a polyAT sequence of 120 nucleotides in length as the 5′ spacer element, and a translation enhancer with one of the following motifs as shown in Table 22 at the 3′ UTR:

TABLE 22
Translation Enhancers

| Translation Enhancer | SEQ ID NO |
|---|---|
| 12S | 178 |
| AES | 176 |
| DDB2 | 171 |
| FcIgG | 174 |
| HBA13UTR | 173 |
| LSP1 | 175 |
| TP53I3 | 172 |

A DNA construct without a translation enhancer was also included.

Circular RNAs were generated by self-splicing using a method described herein. Purified circular RNAs (0.025 picomoles) were transfected into A549 cells (10,000 cells per well in a 96 well plate in media) using LIPOFECTAMINE® MessengerMAX transfection reagent (Invitrogen) according to manufacturer's instructions. MessengerMax alone was used as a control (Carrier only).

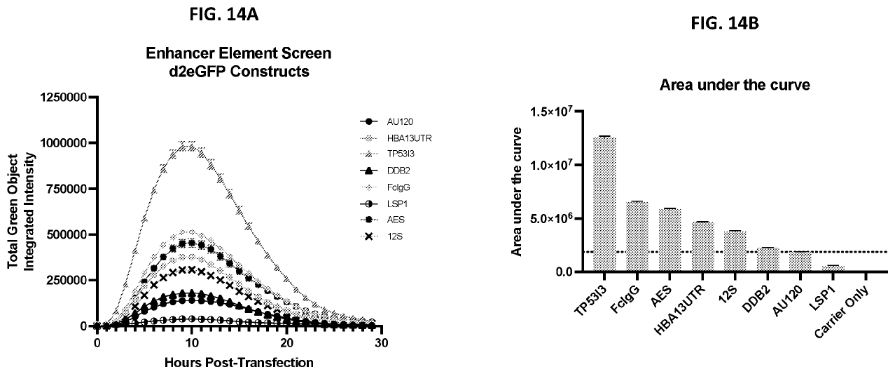

To measure expression of circular RNA encoding EGFPd2, the cells were imaged every hour for 28 hours using a live-cell analysis system (Incucyte® instrument, Sartorius). The time course of expression data is shown in FIGS. 14A and 14B. FIG. 14A shows the EGFPd2 expression over time; FIG. 14B shows the area under the curve from 0 to 24 hours. In this experiment, the 12S, AES, FcIgG, HBA13UTR and TP5313 translation enhancers increased expression relative to having a polyAU120 spacer element at the 3′ position.

### Experiment 2: hEPO

The DNA constructs included a modified CVB3 IRES, an hEPO nucleotide sequence as the ORF, a polyAT sequence of 120 nucleotides in length as the 5′ spacer element, and a translation enhancer with one of the following motifs as shown in Table 23 at the 3′ UTR:

TABLE 23
Translation Enhancers

| Translation Enhancer | Category |
|---|---|
| Ad2VAI | Suppresses immune activation |
| CVB3UTR | Viral UTR |
| DDB2 | Stabilizes mRNA when in 3′ UTR |
| Dengue3UTR | Viral UTR |
| HBA13 | 3′ UTR from stable mRNA |
| Histone4E | Binds elF4E |
| HBVPRE (Hepatitis B post- | Enhances mRNA expression |
| transcriptional regulatory | |
| element) | |
| RotaVP63UTR | Enhances mRNA expression |
| TBR1 | Enhances cap-independent translation |
| TBR2 | Enhances cap-independent translation |
| TBR3 | Enhances cap-independent translation |
| TBR4 | Enhances cap-independent translation |
| TBR5 | Enhances cap-independent translation |
| TBR6 | Enhances cap-independent translation |
| TBR7 | Enhances cap-independent translation |
| TBR8 | Enhances cap-independent translation |
| TBR9 | Enhances cap-independent translation |
| TBR10 | Enhances cap-independent translation |
| TBR11 | Enhances cap-independent translation |
| TBR12 | Enhances cap-independent translation |
| TBR13 | Enhances cap-independent translation |
| TBR15 | Enhances cap-independent translation |
| TBR16 | Enhances cap-independent translation |
| TBR18 | Enhances cap-independent translation |
| TBR19 | Enhances cap-independent translation |
| TBR20 | Enhances cap-independent translation |
| TBR21 | Enhances cap-independent translation |
| TBR22 | Enhances cap-independent translation |
| TBR24 | Enhances cap-independent translation |
| TBR25 | Enhances cap-independent translation |
| TBR26 | Enhances cap-independent translation |
| TBR27 | Enhances cap-independent translation |
| TBR28 | Enhances cap-independent translation |
| TBR29 | Enhances cap-independent translation |
| TP53I3 | Stable mRNA 3′ UTR |

Additional DNA constructs included a translation enhancer with one of the following motifs as shown in Table 24 at either the 5′ UTR or the 3′ UTR:

TABLE 24
Translation Enhancers

| Translation Enhancer | Location |
|---|---|
| BYDV5UTR | 5′ UTR |
| BYDV3UTR | 3′ UTR |
| Dengue5UTR | 5′ UTR |
| Dengue3UTR | 3′ UTR |
| SDV5UTR | 5′ UTR |
| SDV3UTR | 3′ UTR |
| TMV5UTR | 5′ UTR |
| TMV3UTR | 3′ UTR |
| TNVD5UTR | 5′ UTR |
| TNVD3UTR | 3′ UTR |
| TYMV5UTR | 5′ UTR |
| TYMV3UTR | 3′ UTR |
| WestNile5UTR | 5′ UTR |
| WestNile3UTR | 3′ UTR |
| YFV5UTR | 5′ UTR |
| YFV3UTR | 3′ UTR |
| Zika5UTR | 5′ UTR |
| Zika3UTR | 3′ UTR |

DNA constructs without a translation enhancer were also included.

Circular RNAs were generated by self-splicing using a method described herein. Purified circular RNAs (0.025 picomoles) were transfected into A549 cells (10,000 cells per well in a 96 well plate in media) using LIPOFECTAMINE® MessengerMAX transfection reagent (Invitrogen) according to manufacturer's instructions. MessengerMax alone was used as a control (Carrier only).

To measure expression of circular RNA encoding hEPO, supernatant was collected at 24 hours post transfection. Expression was measured using a hEPO-specific ELISA.

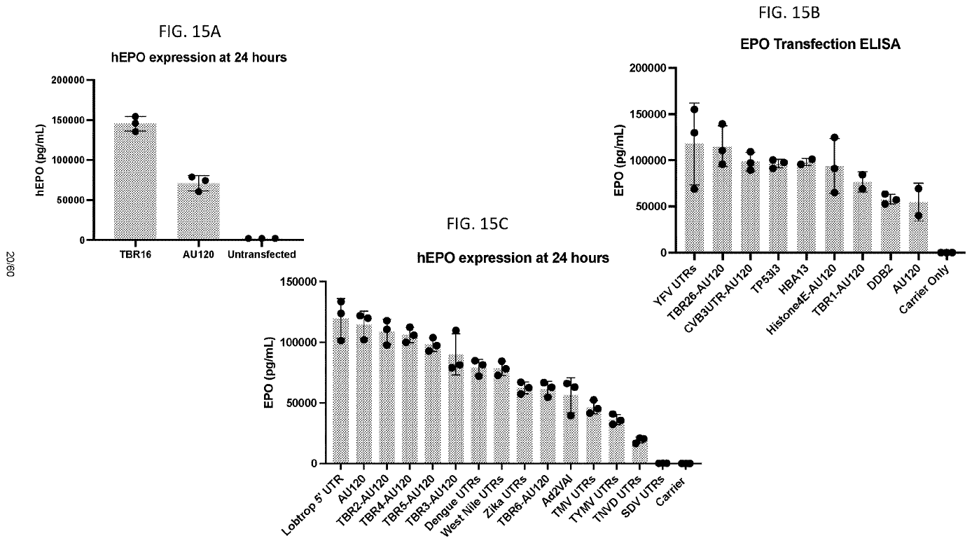

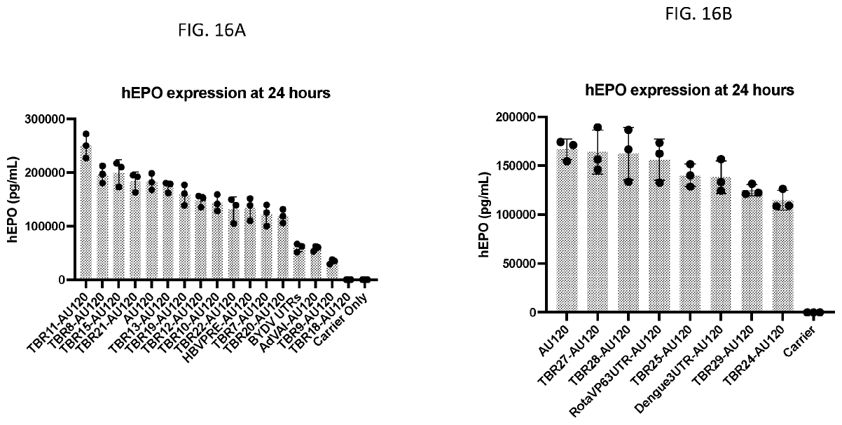

The results of this experiment are shown in FIGS. 15A-15C and 16A-16B. FIG. 15A shows that higher expression was obtained with circular RNAs including the TBR16 translation enhancer at the 3′ UTR compared to a circular RNA without a translation enhancer but includes a 3′ polyAU spacer element. FIG. 15B shows that higher expression was obtained with circular RNAs including the TBR26, CVB3UTR, TP5313, HBA13, Histone4E, or TBR1 translation enhancer at the 3′ UTR compared to a circular RNA without a translation enhancer but includes a 3′ polyAU spacer element. FIG. 15B also shows that higher expression was obtained with: (i) circular RNAs including the YFV translation enhancer at the 5′ UTR compared to a circular RNA without a translation enhancer but includes a 5′ polyAU spacer element; and (ii) circular RNAs including the YFV translation enhancer at the 3′ UTR compared to a circular RNA without a translation enhancer but includes a 3′ polyAU spacer element. FIG. 16A shows that higher expression was obtained with circular RNAs including the TBR11 translation enhancer at the 3′ UTR compared to a circular RNA without a translation enhancer but includes a 3′ polyAU spacer element.

### Experiment 3: FIX-Albumin-HiBIT Data

The DNA constructs included a modified CVB3 IRES, a Factor 9-Albumin-HiBiT nucleotide sequence as the ORF, a polyAT sequence of 120 nucleotides in length as the 5′ and 3′ spacer elements, and a translation enhancer with one of the following motifs as shown in Table 25 at the 3′ UTR:

TABLE 25
Translation Enhancers
Translation enhancer

BBSV (Broad bean stain virus)
BYDV (Barley yellow dwarf virus)
CABYV-X (Cucurbit aphid-borne yellow virus)
CarMV (Carnation mottle virus)
CbMV (Calibrachoa mottle virus)
CBV (Cucumber Bulgarian virus)
CCFV (Cardamine chlorotic virus)
CMMV (Cocksfoot mild mosaic virus)
CNV (Cucumber necrosis virus)
CymRSV (Cymbidium ringspot virus)
ELV (Elderberry latent virus)
GALV (Grapevine Algerian latent virus)
GaMV (Galinsoga mosaic virus)
GoMVA (Gompholobium virus A)
GRV (Groundnut rosette virus)
HCRSV (Honeysuckle chlorotic ringspot virus)
HnRSV (Honeysuckle ringspot virus)
JCSMV (Johnsongradd chlorotic stripe mosaic virus)
LNV (Lisianthus necrosis virus)
LWSV (Leek white stripe virus)
MCMV (Maize chlorotic mottle virus)
MNeSV (Maize necrotiv streak virus)
MNSV264 (Melon necrotic spot virus)
MWLMV (Maize white line mosaic virus)
OLV1 (Olive latent virus)
PCRPV (Pelargonium chlorotic ring pattern virus)
PeLV (Pear latent virus)
PelRSV (Pelargonium ring spot virus)
PEMV2 (Pea enation mosaic virus RNA 2)
PLPV (Pelargonium line pattern virus)
PMV (Panicum mosaic virus)
PNSV (Pelargonium necrotic spot virus)
RrLDV (Rosa rugosa leaf distortion virus)
SCV (Saguaro cactus virus)
STNV (Satellite tobacco necrosis virus)
TBTV (Tomator bushy stunt virus)
TCV (Turnip crinkle virus)
TPAV (Thin paspalum asymptomatic virus)
TYMV (Turnip yellow mosaic virus)

DNA constructs without the translation enhancer were also included.

Circular RNAs were generated by self-splicing using a method described herein. Purified circular RNAs (0.025 picomoles) were transfected into HEK cells (10,000 cells per well in a 96 well plate in media) using LIPOFECTAMINE® MessengerMAX transfection reagent (Invitrogen) according to manufacturer's instructions. MessengerMax alone was used as a control (Carrier only).

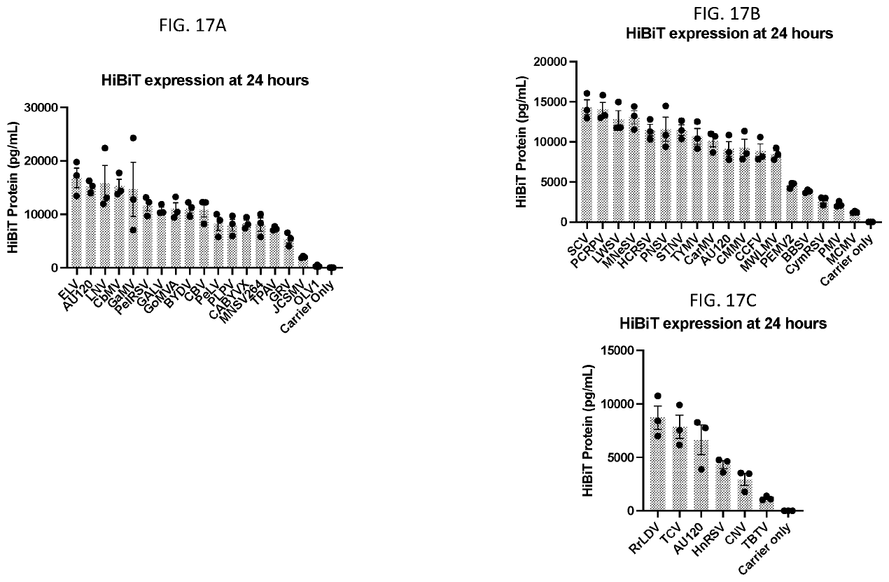

HiBiT expression was measured at 24 hours. The results of this experiment are shown in FIGS. 17A-17C. Higher expression was obtained with circular RNAs including a SCV, PCRPV, LWSV, MneSV, HCRSV, or PNSV translation enhancer at the 3′UTR compared to a circular RNA without a translation enhancer but includes a 3′ polyAU spacer element.

### Experiment 4: ElF4 g Aptamer

The DNA constructs included an EMCV IRES, a Gluc nucleotide sequence as the ORF, a polyA sequence of 50 nucleotides in length as the 5′ spacer element, and two copies of an elF4 g aptamer as the translation enhancer with one of the following nucleotide sequences as shown in Table 26:

TABLE 26
Translation Enhancers

| Translation enhancer | Sequence (5′ to 3′) |
|---|---|
| Full length eIF4g aptamer | aaaaaagggactcactatttgttttcgcgcccagttgcaaaaagtgtcgccg |
| | catctagagggccaaaaaagggactcactatttgttttcgcgcccagttgca |
| | aaaagtgtcgccgcatctagagggccaaaaaa (SEQ ID NO: 253) |
| Full length eIF4g scrambled | aaaaaaccgatcactaaaacagctcgtagtccgtttttcttattgtctgcggtt |
| aptamer | gagtcgctgaacaccaaccaaaaaaccgatcactaaaacagctcgtagt |
| | ccgtttttcttattgtctgcggttgagtcgctgaacaccaaccaaaaaa |
| | (SEQ ID NO: 254) |
| eIF4g minimized aptamer | aaaaaaactcactatttgttttcgcgcccagttgcaaaaagtgtcgaaaaaa |
| | actcactatttgttttcgcgcccagttgcaaaaagtgtcgaaaaaa (SEQ |
| | ID NO: 255) |
| eIF4g scrambled minimized | aaaaaagtcagtaccatcacggaattattctcgcttatggcctgataaaaaa |
| aptamer | gtcagtaccatcacggaattattctcgcttatggcctgataaaaaagtcagta |
| | ccatcacggaattattctcgcttatggcctgataaaaaa (SEQ ID NO: |
| | 256) |

Circular RNAs were generated by self-splicing using a method described herein. Purified circular RNAs (0.1, 0.5, or 1 picomoles) were transfected into Hela cells (10,000 cells per well in a 96 well plate in media) using LIPOFECTAMINE® MessengerMAX transfection reagent (Invitrogen) according to manufacturer's instructions.

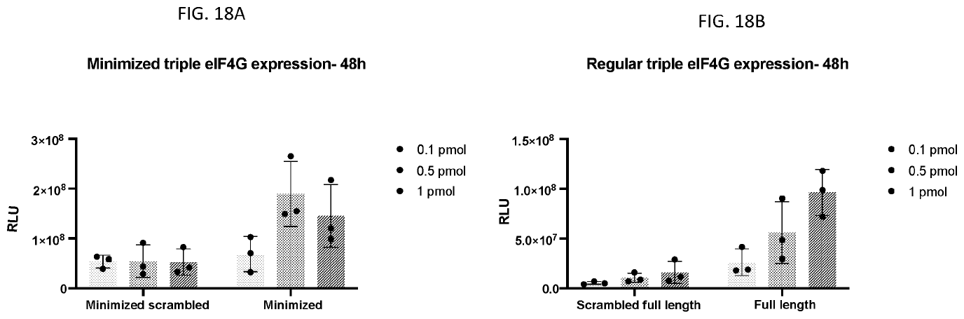

Gluc expression was measured at 24 and 48 hours. The results of this experiment are shown in FIGS. 18A and 18B. The addition of either minimized or full length elF4 g aptamer at the 5′ UTR increased Gluc expression relative to the addition of the scrambled aptamer.

### Example 12: Effect of Spacers and Translation Enhancers on Circular RNA Expression In Vitro

This example demonstrates the effect of spacer elements and translation enhancers on expression of CFTR from circular RNAs in mammalian cells.

Circular RNAs were designed to include translation enhancers at the 5′ UTR or the 3′ UTR, an IRES and a nucleotide sequence encoding a CFTR. In this example, DNA constructs were designed to include, from 5′ to 3′: 5′ spacer element, IRES, ORF, and translation enhancer. The DNA constructs included a modified CVB3 IRES, a CFTR nucleotide sequence as the ORF, a polyAT sequence of 120 nucleotides in length as the 5′ spacer element, and a translation enhancer sequence with one of the following motifs as shown in Table 27 at the 3′ UTR:

TABLE 27
Translation Enhancer
Translation Enhancer

FcIgG
12S
TPAV
DRB4
TPAV-AU120
AES
LSP1
HBA13UTR
TP53I3
AU120-TPAV
DDB2

A DNA construct without a translation enhancer was also included.

Circular RNAs were produced by self-splicing using a method described herein. Purified circular RNAs (2.5 μg) were transfected into HEK293T cells (1 million cells per well in a 96 well plate in media) using LIPOFECTAMINE® MessengerMAX transfection reagent (Invitrogen) according to manufacturer's instructions.

CFTR expression was measured at 24 hours by Western blot.

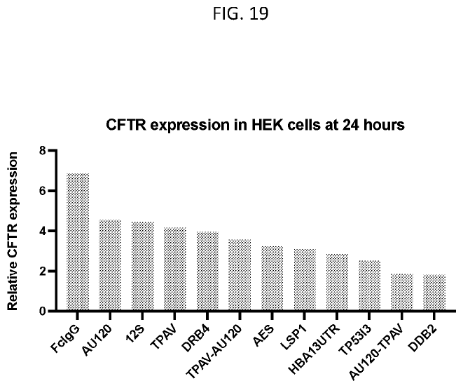

The results of this experiment are shown in FIG. 19. In this example, elevated expression was obtained with circular RNAs including a FcIgG translation enhancer at the 3′ UTR compared to a circular RNA without a translation enhancer but includes a polyAU120 spacer element.

### Example 13: Effect of Spacers and Translation Enhancers on Circular RNA Expression In Vitro

This example demonstrates the effect of spacer elements and translation enhancers on expression of a polypeptide from circular RNAs in mammalian cells.

Circular RNAs were designed to include translation enhancers at the 5′ UTR or the 3′ UTR, an IRES and a nucleotide sequence encoding a polypeptide. In this example, DNA constructs were designed to include, from 5′ to 3′: 5′ spacer element, IRES, ORF, and translation enhancer. The DNA constructs included a modified CVB3 IRES, a nucleotide sequence encoding a polypeptide as the ORF, a polyAT sequence of 120 nucleotides in length as the 5′ spacer element, and a heterogenous translation enhancer sequence with one of the following motifs shown in Table 28 at the 3′ UTR:

TABLE 28
Translation Enhancers
Translation Enhancer

TBR16
Histone4e
RnaseLInh
TBR26
CVB3UTR
TP5313
TPAV
DRB4
TYFVUTRs
HBA13
TBR1

Circular RNAs were produced by self-splicing using a method described herein. Purified circular RNAs (0.08 μmol) were transfected into HEK293T cells (30K cells per well in a 96 well plate in media) using LIPOFECTAMINE® MessengerMAX transfection reagent (Invitrogen) according to manufacturer's instructions. MessengerMax alone was used as a control (Blank). The polypeptide expression was measured by flow cytometry.

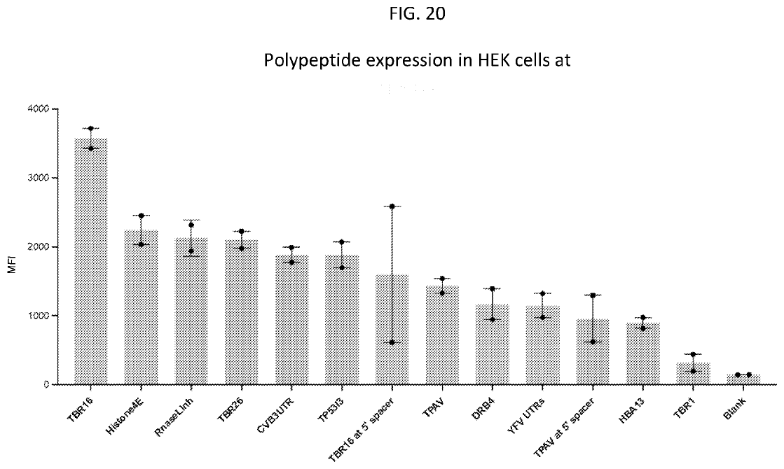

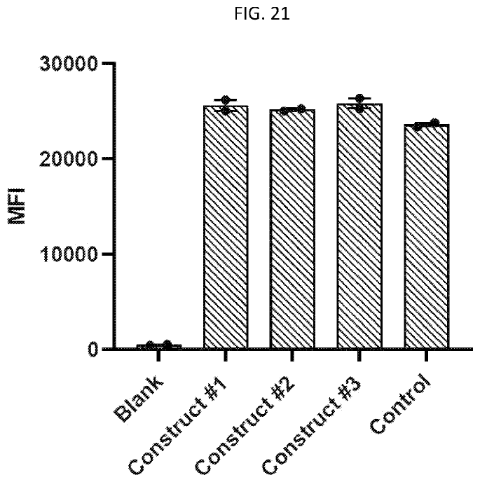

The results of this experiment are shown in FIG. 20. In this example, TBR16 in the 3′ UTR had the greatest impact on the polypeptide expression.

### Example 14: Effect of Spacers and Translation Enhancers on Circular RNA Expression In Vitro

This example demonstrates the effect of spacer elements and translation enhancers on expression of polypeptides from circular RNAs in mammalian cells.

Circular RNAs were designed to include an IRES and nucleotide sequence encoding a polypeptide. DNA constructs were designed to include, from 5′ to 3′:
(i) 5′ spacer element, IRES, ORF, and 3′ spacer element; or
(ii) 5′ spacer element, IRES, ORF, translation enhancer, and 3′ spacer element.

In this example, the DNA constructs included a modified CVB3 IRES or EV71 IRES, a nucleotide sequence encoding a polypeptide as the ORF, a polyAT sequence of 120 nucleotides in length as the spacer elements, and TPVA as the translation enhancer. Exemplary constructs are shown in Table 29.

TABLE 29
Exemplary Constructs

| | 5′ | | | Translation | 3′ |
|---|---|---|---|---|---|
| | spacer | IRES | ORF | Enhancer | spacer |
| Construct #1 | AT120 | modified | polypeptide | None | AT120 |
| | | CVB3 | | | |
| Construct #2 | AT120 | modified | polypeptide | TPVA | AT120 |
| | | CVB3 | | | |
| Construct #3 | AT120 | EV71 | polypeptide | None | AT120 |
| Control | None | modified | polypeptide | None | None |
| | | CVB3 | | | |

Circular RNAs were generated by self-splicing using a method described herein. Unmodified linear RNA was synthesized by in vitro transcription using T7 RNA polymerase from a DNA template including the motifs listed above in the presence of 7.5 mM of NTP. Template DNA was removed by treating with DNase. Synthesized linear RNA was purified with an RNA clean up kit (New England Biolabs, T2050). Circular RNAs were purified anionic exchange chromatography.

Circular RNAs (0.3 picomoles) were transfected into HEK293T cells (30,000 cells per well in a 96 well plate in serum-free media) using LIPOFECTAMINE® MessengerMAX transfection reagent (Invitrogen) according to manufacturer's instructions. MessengerMax alone was used as a control (Blank). Cells were harvested 24 hours post-transfection and polypeptide expression on the cell surface was measured by flow cytometry. Briefly, cells were first harvested and stained with a Live/Dead stain. Cells were then treated with a mouse anti-polypeptide primary antibody followed by a phycoerythrin (PE)-tagged anti-IgG antibody. Expression of polypeptide was measured as a function of PE mean fluorescence intensity.

FIG. 21 shows that including a translation enhancer (Construct #2) increased expression relative to the Control (One-way ANOVA, P value of 0.0238). Expression of Construct #3 was comparable to the Control (One-way ANOVA, P value of 0.0613 (ns)). FIG. 21 also shows that comparable expression profiles were obtained with Construct #1 (modified CVB3 IRES) and Construct #3 (EV71 IRES).

### Example 15: Effect of RNA Stability Elements on Circular RNA Stability

This example describes the effect of RNA stability on the stability of circular RNAs in mammalian cells.

DNA constructs are designed to include an IRES, an ORF, and an RNA stability element either before the IRES or after the ORF, or in both sides. DNA constructs are designed to include an CVB3 IRES and a Gluc nucleotide sequence as the ORF.

Circular RNAs are generated by self-splicing using a method described herein. Purified circular RNAs (0.025 picomoles) are transfected into HEK293T cells (10,000 cells per well in a 96 well plate in antibiotics-free media) using LIPOFECTAMINE® MessengerMAX transfection reagent (Invitrogen) according to the manufacturer's instructions. MessengerMax alone is used as a control (No RNA). To allow time-course monitoring of RNA stability, 4 plates of cells are prepared at 4 hr, 24 hr, 48 hr and 72 hr, respectively.

RNAs from the cells are harvested at each time point with CellsDirect one step qRT-PCR kits (Invitrogen) according to the manufacturer's instruction. To measure RNA levels, harvested RNAs are subjected to RT-qPCR with Luna Universal probe one-step RT-qPCR kit. The level of circular RNA is measured with the primer against the CVB3 IRES. Beta actin mRNA is used as control. The primers used are described in Table 30.

TABLE 30
Primers

| Name | Sequence |
|---|---|
| Beta-Actin | 5′-CCTTGCACATGCCGGAG-3′ (SEQ ID NO: 269) |
| Forward | |
| Beta-Actin | 5′-ACAGAGCCTCGCCTTTG-3′ (SEQ ID NO: 270) |
| Reverse | |
| Beta-Actin | /56-FAM/ATGGATGAT/ZEN/GATATCGCCGCGCTC/3IABkFQ/ (SEQ ID NOS: |
| Probe | 259 and 260) |
| CVB3 IRES | 5′-CCTGAATGCGGCTAATCCTAA-3′ (SEQ ID NO: 261) |
| Forward | |
| CVB3 IRES | 5′-AACACGGACACCCAAAGTAG-3′ (SEQ ID NO: 262) |
| Reverse | |
| CVB3 IRES | /5HEX/AGTGTGTCG/ZEN/TAACGGGCAACTCTG/3IABkFQ/ (SEQ ID NOS: |
| Probe | 263 and 264) |

The level of circular RNA (measured as CVB3 IRES) is normalized by the level of beta-actin. To monitor time-course RNA stability, the level of circular RNA at the 4 hr time point is set to 1 and the other time points are normalized to the 4 hr time point.

### Example 16. Effect of RNA Stability Elements on Circular Polyribonucleotide Stability

This example demonstrates the effect of RNA stability elements on the stability of circular polyribonucleotides in mammalian cells over time.

DNA constructs were designed to include from 5′ to 3′: a 5′ spacer element, an IRES, an ORF, and a 3′ RNA stability element, wherein the IRES was a modified CVB3 IRES, the ORF encoded polypeptide E, the 5′ spacer element was a poly-AT sequence of 120 nucleotides in length, and the 3′ RNA stability element included one of the following motifs described in Table 31, below.

TABLE 31
RNA Stability Elements

| RNA Stability | SEQ |
|---|---|
| Element | ID NO |
| CAPG | 294 |
| FTL | 302 |
| MYL6B | 311 |
| PRDX5 | 319 |
| TMEM258 | 327 |
| CKB | 295 |
| GPX4 | 304 |
| RABAC1 | 320 |
| TRAPPC6A | 328 |

A DNA construct (AU120) was also designed that included from 5′ to 3′: a 5′ spacer element, an IRES, an ORF, and a 3′ spacer element, wherein the IRES was a modified CVB3 IRES, the ORF encoded polypeptide E, and the spacer elements were poly-AT sequences of 120 nucleotides in length.

Circular RNAs were generated by self-splicing using a method described herein. Purified circular RNAs (2 picomoles per well) were electroporated into A549 cells (50,000 cells per well in a 96 well plate in media). To allow time-course monitoring at 2 hours, 24 hours, and 96 hours, 3 plates of cells were prepared. Following electroporation, cells sat for about 5 minutes before 100 μL of media was added. This mixture was then transferred to a new plate for each timepoint. The 2-hour timepoint was transferred to a V-bottom plate. For the 2-hour timepoint, the V-bottom plate was spun down 20 minutes prior to timepoint collection. Samples were washed once with PBS, spun again, and then lysed. For all other timepoints, the media was removed, cells were washed once with PBS, and then lysed.

RNA was extracted. For each timepoint, 30 μL of lysis media was added, and the samples were allowed to lyse on ice for 10 minutes. Lysates were then transferred to 96-well plates and incubated at 75° C. for 10 minutes. 2 L of each sample was then used for qPCR.

qPCR measurements were carried out using the Luna Universal Probe qPCR Master Mix and a qPCR primer/probe set that targeted the IRES in a total sample volume of 15 μL. Cq values were converted to absolute pmol amounts using standard curves that were run along with the samples. Standard curves were prepared from 4 randomly selected circRNAs, and were evaluated using the same qPCR master mix as the samples. Standard curves were each composed of seven serial 10-fold dilutions, with the first sample in each standard curve containing 13.3 fmol of circRNA. Linear curves were used to fit the relationship between the Cq values for each standard curve and the log-transformed pmol quantities of RNA used for each sample. The fit slopes and γ-intercepts fit for each of the four standard curves were then averaged, and these average values were used for the calculations converting the sample Cq values to absolute pmol amounts.

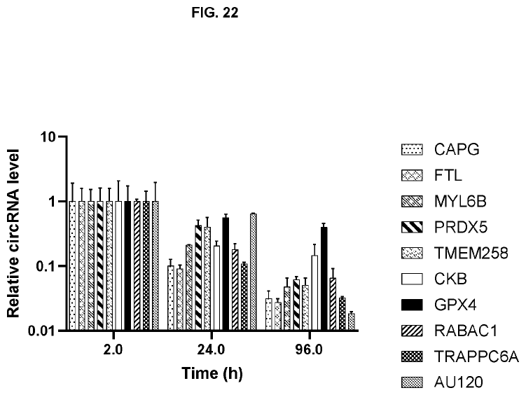

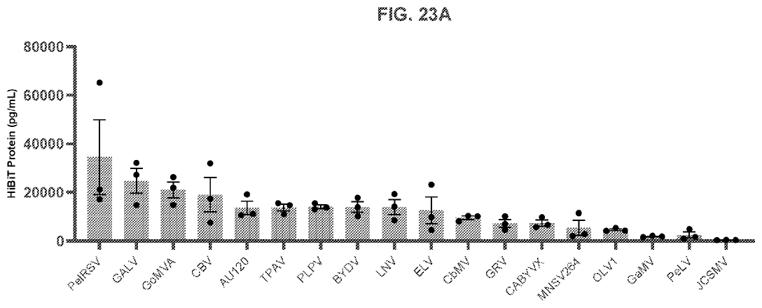

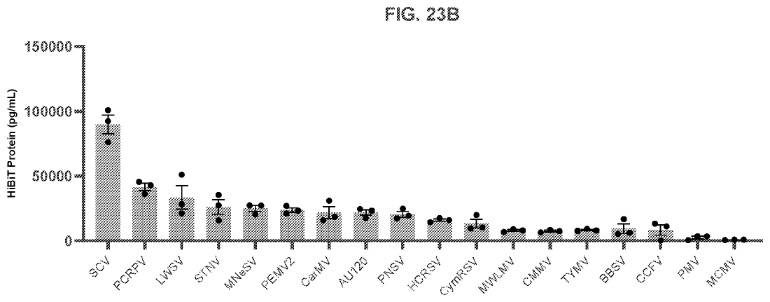

FIG. 22 shows relative circRNA abundance over time. For each circRNA, timepoints were scaled relative to the 2 hour timepoint. FIG. 22 shows circular polyribonucleotides that have increased stability relative to AU120.

### Example 17. Effect of Translation Enhancers on Polypeptide Expression

This example demonstrates the effect of translation enhancers from plant viral 3′ UTRs on expression of a polypeptide from circular RNAs in mammalian cells.

DNA constructs were designed to include from 5′ to 3′: (i) a 5′ spacer element, (ii) IRES, (iii) ORF, and (iv) a 3′ translation enhancer, wherein the 5′ spacer element was a poly-AT sequence of 120 nucleotides in length (SEQ ID NO: 335), the IRES was a modified CVB3 IRES, the ORF encoded Factor 9-HiBiT, and the 3′ translation enhancer included one of the following motifs as shown in Table 32.

TABLE 32
Translation Enhancers

| Translation | SEQ |
|---|---|
| Enhancer | ID NO |
| TPAV | 38 |
| JCSMV | 51 |
| PLPV | 24 |
| GALV | 60 |
| CABYVX | 68 |
| PelRSV | 28 |
| GoMVA | 52 |
| BYDV | 1 |
| CbMV | 29 |
| CBV | 49 |
| ELV | 26 |
| LNV | 63 |
| PelV | 62 |
| GaMV | 36 |
| GRV | 8 |
| MNSV264 | 48 |
| OLV1 | 3 |
| MCMV | 42 |
| MWLMV | 50 |
| CMMV | 37 |
| HCRSV | 35 |
| SCV | 30 |
| PNSV | 59 |
| LWSV | 4 |
| MNeSV | 47 |
| PEMV2 | 40 |
| STNV | 23 |
| BBSV | 10 |
| PCRPV | 25 |
| PMV | 41 |
| CCFV | 67 |
| TYMV | 64 |
| CymRSV | 56 |
| CarMV | 32 |
| TBTV | 7 |
| RrLDV | 27 |
| HnRSV | 33 |
| CNV | 57 |
| TCV | 66 |

A DNA construct without a 3′ translation enhancer was also included (AU120). This construct included a 5′ spacer element, an IRES, an ORF, and a 3′ spacer element, where the spacer elements were poly-AT sequences of 120 nucleotides in length (SEQ ID NO: 335), the IRES was a modified CVB3 IRES, and the ORF encoded Factor 9-HiBiT.

Circular RNAs were generated by self-splicing using a method described herein. Purified circular RNAs (0.025 picomoles) were transfected into A549 RIG-KO cells (10,000 cells per well in a 96 well plate in media) using LIPOFECTAMINE® MessengerMAX transfection reagent (Invitrogen) according to manufacturer's instructions.

To measure expression of circular RNA encoding Factor 9-HiBiT, supernatant was collected at 24 hours post transfection. Expression was measured by measuring HiBiT expression using the Nano-Glo® HiBiT Extracellular Detection System (Promega).

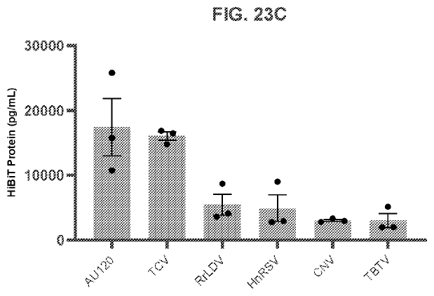

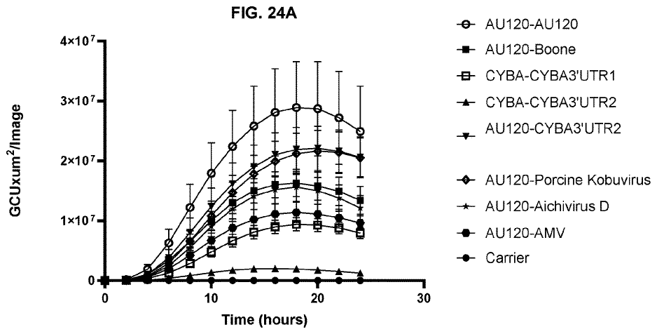

The results of this experiment are shown in FIGS. 23A-23C. We observed that some plant viral 3′ UTRs included at the 3′ UTR increased expression of Factor 9-HiBiT from circular RNAs compared to expression from a circular RNA without a translation enhancer but included a 3′ polyAU spacer element at the 3′ UTR. FIG. 23A shows that increased expression was obtained with circular RNAs including a GALV 3′ UTR, PelRSV 3′ UTR, GoMVA 3′ UTR, or CBV 3′ UTR translation enhancer at the 3′ UTR compared to a circular RNA without a translation enhancer but includes a 3′ polyAU spacer element. FIG. 23B shows increased expression was obtained with circular RNAs including a SCV 3′ UTR, PCRPV 3′ UTR or LWSV 3′ UTR compared to a circular RNA without a translation enhancer but includes a 3′ polyAU spacer element. FIG. 23C shows similar expression was obtained with circular RNAs including at TCV 3′ UTR compared to a circular RNA without a translation enhancer but includes a 3′ polyAU spacer element.

### Example 18. Effect of Spacers and Translation Enhancers on Polypeptide Expression

This example demonstrates the effect of spacer elements and translation enhancers on expression of a polypeptide from circular RNAs in mammalian cells.

In this example, DNA constructs were designed to include an IRES, an ORF, and
(i) both a 5′ spacer element and a 3′ spacer element (“dual spacer design”);
(ii) a 5′ spacer element and a 3′ translation enhancer;
(iii) a 5′ translation enhancer and a 3′ spacer element; or
(iv) A 5′ translation enhancer and a 3′ translation enhancer.
   DNA constructs included a modified CVB3 IRES, a nucleotide sequence encoding EGFPd2 as the ORF, and spacer elements and/or translation enhancers as shown in Table 33.

TABLE 33
Spacer elements and Translation Enhancers

| 5′ spacer element | 3′ spacer element |
|---|---|
| or 5′ translation | or 3′ translation |
| enhancer | enhancer |
| AU120 | AU120 |
| AU120 | CYBA 3′UTR2 |
| AU120 | Porcine Kobuvirus |
| AU120 | Boone |
| AU120 | Aichivirus D |
| AU120 | AMV |
| CYBA | CYBA 3′UTR1 |
| CYBA | CYBA 3′UTR2 |

TABLE 34
Spacer Elements and Translation Enhancers

| Spacer Element | |
|---|---|
| or Translation | |
| Enhancer | SEQ ID NO |
| AU120 | 335 |
| CYBA | CGCGCCTAGCAGTGTCCCAGCCGGGTTCGTGTCGCC (SEQ ID NO: 344) |
| CYBA 3′ UTR1 | CCTCGCCCCGGACCTGCCCTCCCGCCAGGTGCACCCACCTGCAATAAATGCAGCGA |
| | AGCCGGGA (SEQ ID NO: 288) |
| CYBA 3′ UTR2 | CCTCGCCCCGGACCTGCCCTCCCGCCAGGTGCACCCACCTGCAATAAATGCAGCGA |
| | AGCCGGGACCTCGCCCCGGACCTGCCCTCCCGCCAGGTGCACCCACCTGCAATAA |
| | ATGCAGCGAAGCCGGGA (SEQ ID NO: 289) |
| Boone | 136 |
| Porcine Kobuvirus | 143 |
| Aichivirus D | 144 |
| AMV | 145 |

Circular RNAs were generated by self-splicing using a method described herein. Purified circular RNAs (0.025 picomoles) were transfected into A549 cells (10,000 cells per well in a 96 well plate in media) using LIPOFECTAMINE® MessengerMAX transfection reagent (Invitrogen) according to manufacturer's instructions. MessengerMax alone was used as a control (Carrier only).

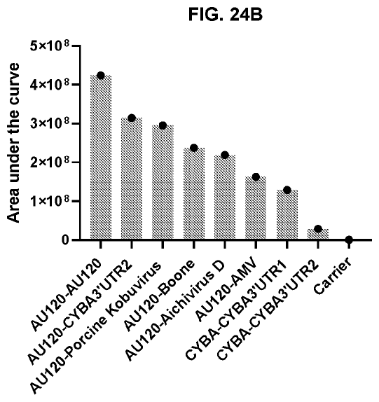

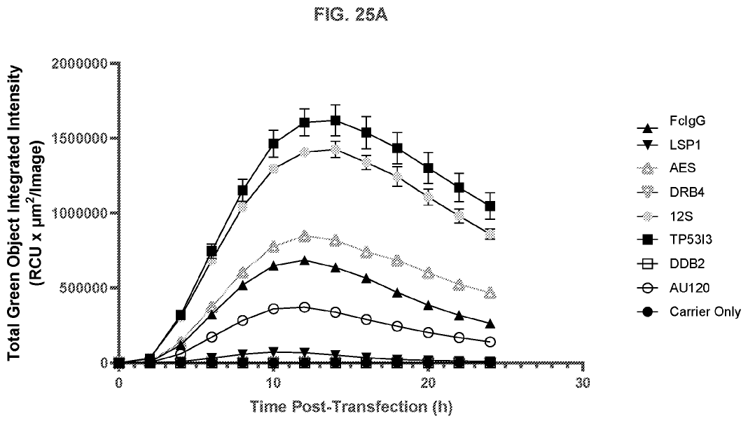

To measure expression of circular RNA encoding EGFPd2, the cells were imaged every hour for 28 hours using a live-cell analysis system (Incucyte® instrument, Sartorius). The time course of expression data is shown in FIGS. 24A and 24B. FIG. 24A shows EGFPd2 expression over time; FIG. 24B shows the area under the curve from 0 to 24 hours.

### Example 19. Effect of Translation Enhancer on Circular RNA Expression In Vitro

This example demonstrates the effect of having a 5′ translation enhancer from plant viral 3′ UTRs on expression of a polypeptide from circular RNAs in mammalian cells.

DNA constructs were designed to include from 5′ to 3′: (i) a 5′ translation enhancer, (ii) IRES, (iii) ORF, and (iv) 3′ spacer element, wherein the 3′ spacer element was a poly-AT sequence of 120 nucleotides in length (SEQ ID NO: 335), the IRES was a modified CVB3 IRES, the ORF encoded EGFPd2, and the 5′ translation enhancer with one of the following motifs, as described in Table 35.

TABLE 35
Translation Enhancers

| Translation | SEQ |
|---|---|
| Enhancer | ID NO |
| FcIgG | 174 |
| LSP1 | 175 |
| AES | 176 |
| DRB4 | 177 |
| 12S | 178 |
| TP5313 | 172 |
| DDB2 | 171 |

A DNA construct without a translation enhancer was also included (AU120). This construct included a 5′ spacer element, an IRES, an ORF, and a 3′ spacer element, where the spacer elements were poly-AT sequences of 120 nucleotides in length (SEQ ID NO: 335), the IRES was a modified CVB3 IRES, and the ORF encoded EGFPd2.

Circular RNAs were generated by self-splicing using a method described herein. Purified circular RNAs (0.025 picomoles) were transfected into A549 cells (10,000 cells per well in a 96 well plate in media) using LIPOFECTAMINE® MessengerMAX transfection reagent (Invitrogen) according to manufacturer's instructions. MessengerMax transfection reagent alone was used as a control (Carrier only).

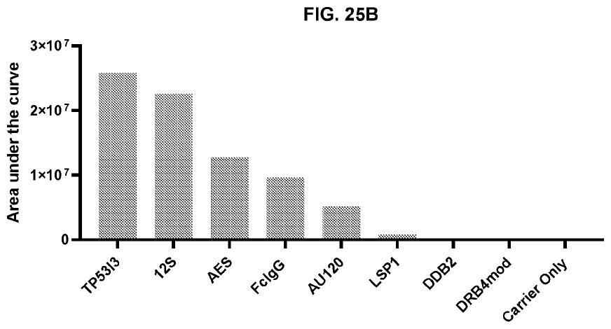

To measure expression of circular RNA encoding EGFPd2, the cells were imaged every hour for 28 hours using a live-cell analysis system (Incucyte® instrument, Sartorius). The time course of expression data is shown in FIGS. 25A and 25B. FIG. 25A shows EGFPd2 expression over time; FIG. 25B shows the area under the curve from 0 to 24 hours. In this experiment, the TP5313, 12S, AES, DRB4, and FcIgG translation enhancers at the 5′ UTR resulted in higher expression relative to having a polyAU120 spacer element at the 5′ position.

### Example 20. Effect of IRES on Polypeptide Expression

This example demonstrates the effect of different IRESes on expression in mammalian cells of a polypeptide from circular RNAs having a 3′ translation enhancer or 3′ spacer element.

DNA constructs were designed to include from 5′ to 3′: (i) a 5′ spacer element, (ii) IRES, (iii) ORF, and (iv) 3′ translation enhancer, wherein the spacer element was a poly-AT sequence of 120 nucleotides in length (SEQ ID NO: 335), IRES was a EV69 IRES or modified CVB3 IRES, the ORF encoded polypeptide B having a HiBiTpeptide tag, and the 3′ translation enhancer included one the following motifs, as shown in Table 36.

TABLE 36
Translation Enhancers

| Translation | SEQ |
|---|---|
| Enhancer | ID NO |
| HBA13 | 173 |
| Histone4E | 72 |
| TBR16 | 90 |

A DNA construct without a translation enhancer was also included (AU120). This construct included a 5′ spacer element, an IRES, an ORF, and a 3′ spacer element, where the spacer elements were poly-AT sequences of 120 nucleotides in length (SEQ ID NO: 335), the IRES was a EV69 IRES or modified CVB3 IRES, and the ORF encoded polypeptide B having a HiBiT peptide tag.

Circular RNAs were generated by self-splicing using a method described herein. Purified circular RNAs (0.025 picomoles) were transfected into A549 cells (10,000 cells per well in a 96 well plate in media) using LIPOFECTAMINE® MessengerMAX transfection reagent (Invitrogen) according to manufacturer's instructions. MessengerMax transfection reagent alone was used as a control (Carrier only).

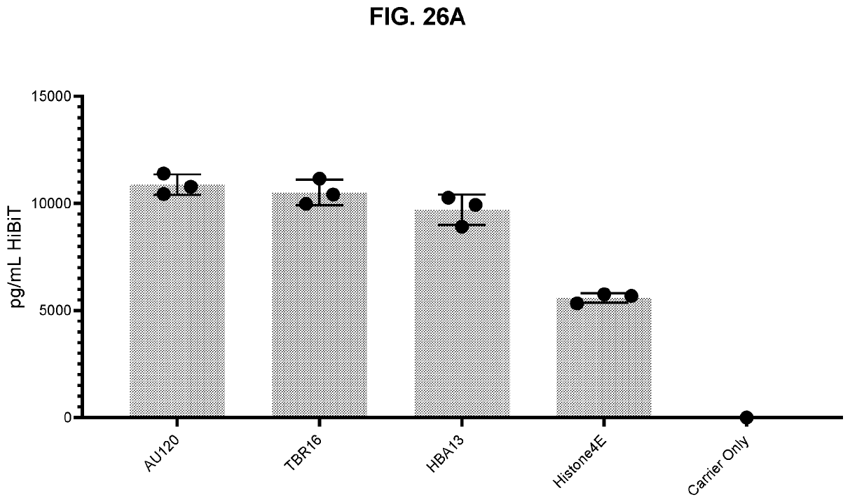

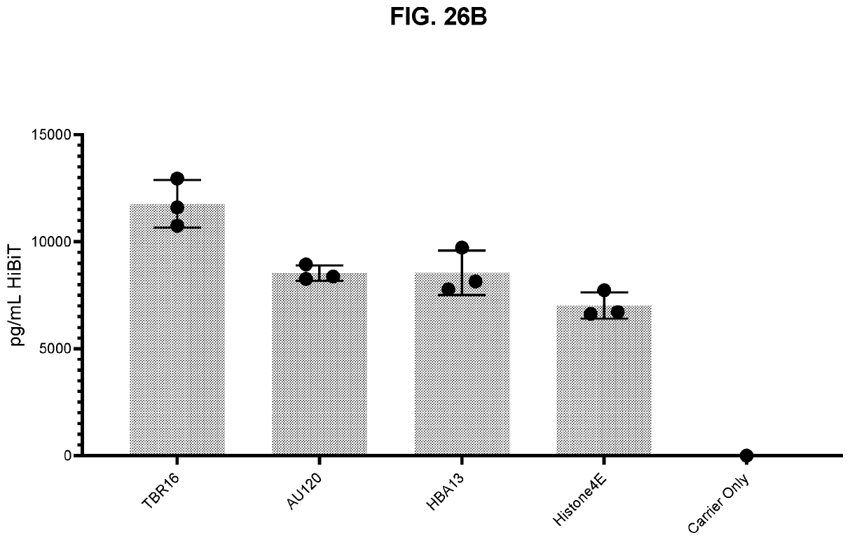

To measure expression of circular RNA encoding a polypeptide B-HiBiT, polypeptide B-HiBiT levels were measured at 24 hours post-transfection by measuring luminescence from the HiBiT tag, using a bioluminescent protein detection assay following manufacturer's instructions (Nano-Glo® HiBiT Extracellular Detection System, Promega Catalog No. N420). The results of this experiment are shown in FIG. 26A (EV69) and FIG. 26B (modified CVB3).

### Example 21. Effect of Multiple Translation Enhancers on Polypeptide Expression

This example demonstrates the effect of having translation enhancers 5′ and 3′ to an ORF encoding a polypeptide, on expression of that polypeptide from circular polyribonucleotides over time.

DNA constructs were designed and included, from 5′ to 3′: (i) a 5′ translation enhancer, (ii) IRES, (iii) ORF, and (iv) 3′ translation enhancer, wherein the IRES was a modified CVB3 IRES, the ORF encoded polypeptide B having a HiBiT peptide tag, and the 5′ and 3′ translation enhancers included one of the following motifs, as shown in in Table 37.

TABLE 37
Translation Enhancers

| Translation | SEQ |
|---|---|
| Enhancers | ID NO |
| DRB4 | 177 |
| 12S | 178 |
| AES | 176 |
| FcIgG | 174 |
| HBA13 | 173 |
| Histone4E | 72 |
| RNaseLInh | 179 |
| TBR16 | 90 |
| TP53I3 | 172 |
| TBR26 | 100 |

A DNA construct without translation enhancers was also included (AU120-AU120). This construct included a 5′ spacer element, an IRES, an ORF, and a 3′ spacer element, where the spacer elements were poly-AT sequences of 120 nucleotides in length (SEQ ID NO: 335), the IRES was a modified CVB3 IRES, and the ORF encoded polypeptide B having a HiBiT peptide tag.

Circular RNAs were generated by self-splicing using a method described herein. Purified circular RNAs (0.025 picomoles) were transfected into A549 cells (10,000 cells per well in a 96 well plate in media) using LIPOFECTAMINE® MessengerMAX transfection reagent (Invitrogen) according to manufacturer's instructions. MessengerMax transfection reagent alone was used as a control (Carrier only).

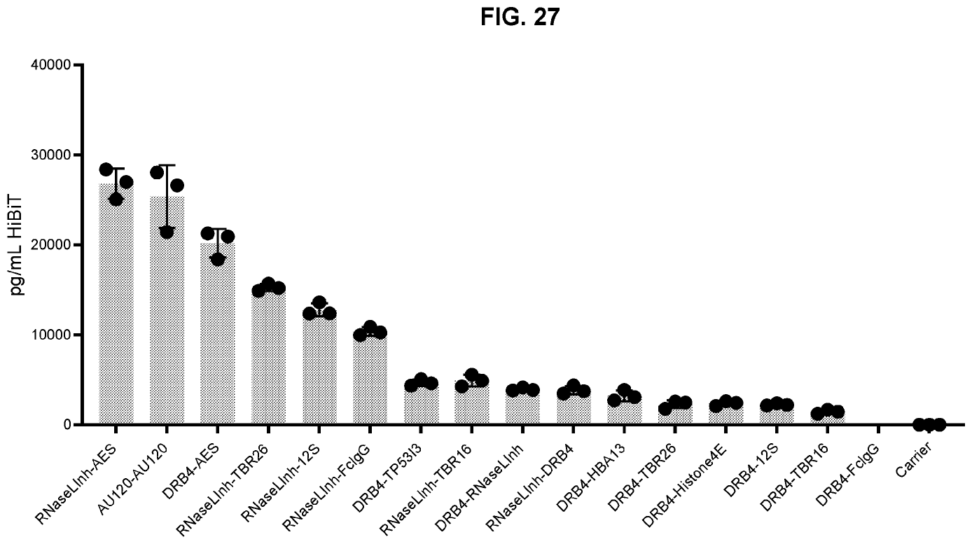

To measure expression of circular RNA encoding a polypeptide B, polypeptide B levels were measured using the HiBiT tag; using a bioluminescent protein detection assay following manufacturer's instructions (Nano-Glo® HiBiT Extracellular Detection System, Promega Catalog No. N420). The results of this experiment are shown in FIG. 27.

### Example 22: Effect of Spacers and Translation Enhancers on Expression of Polypeptides from Circular RNA

This example demonstrates the effect of spacer elements and translation enhancers on expression of polypeptides from circular RNAs in mammalian cells.

Circular RNAs were designed to include an IRES and an ORF. The following DNA constructs were designed and included from 5′ to 3′:
(i) a 5′ spacer element, an IRES, an ORF, and a 3′ spacer element;
(ii) a 5′ translation enhancer, an IRES, an ORF, and a 3′ spacer element;
(iii) a 5′ spacer element, an IRES, an ORF, and a 3′ translation enhancer;
(iv) a 5′ translation enhancer, an IRES, and an ORF.

The DNA constructs included a modified CVB3 IRES, a nucleotide sequence encoding polypeptide G as the ORF, a polyAT sequence of 120 nucleotides in length as the spacer element(s), and a translation enhancer with a motif as shown in Table 38.

TABLE 38
Translation Enhancers

| Translation | SEQ |
|---|---|
| Enhancer | ID NO |
| AP2A2 | 167 |
| CRIP | 166 |
| TRAM1 | 163 |
| VAMP3 | 165 |
| PSMD5 | 168 |

Circular RNAs were generated by self-splicing using a method described herein. Purified circular RNAs (0.1 picomoles) were transfected into HEK293T cells (100,000 cells per well in a 48 well plate) using LIPOFECTAMINE® MessengerMAX transfection reagent (Invitrogen) according to manufacturer's instructions.

24 hours post transfection, protein was purified using RIPA buffer (ThermoFisher, J63306.AP) and HALT (1861281) and quantified using Pierce™ 660 nm Protein Assay Reagent (ThermoFisher, 22660). 0.1 μg/μl protein was run on the Jess using the Protein Simple Separation Module: 25-Capillary Cartridges 12-230 kDa (SM W003). The primary antibody used was a monoclonal anti-polypeptide G antibody and normalized using the Protein Simple Protein normalization module (043-824).

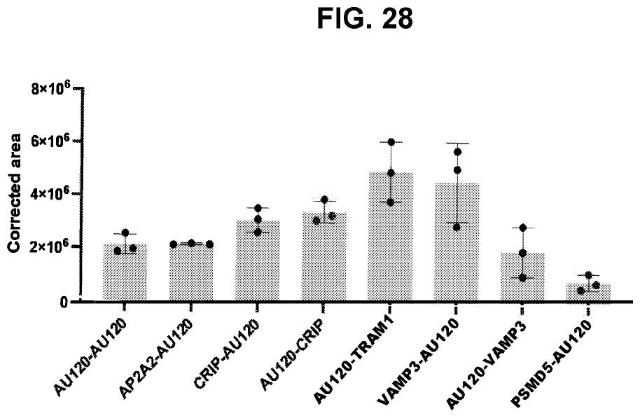

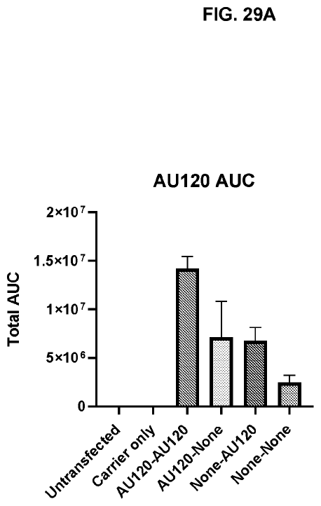

The results of this experiment are shown in FIG. 28.

### Example 23: Effect of a Dual Spacer Design on Polypeptide Expression from Circular RNA

This example demonstrates the effect of a dual spacer design with identical lengths and sequences on expression of ORFs from circular RNAs in mammalian cells.

In this example, DNA constructs were designed to include an IRES, an ORF, and a 5′ spacer element or a 3′ spacer element (“single spacer design”), or both a 5′ spacer element and a 3′ spacer element (“dual spacer design”). DNA constructs without a 5′ or 3′ spacer element served as a control. DNA constructs included a modified CVB3 IRES, a nucleotide sequence encoding GFPd2 or a nucleotide sequence encoding polypeptide B having a HiBiT peptide tag as the ORF, and spacer elements as shown in Table 39.

TABLE 39
Spacer elements

| 5′ spacer | 3′ spacer |
|---|---|
| element | element |
| AU120 | AU120 |
| AU120 | None |
| None | AU120 |
| None | None |

### Experiment 1: GFPd2

Circular RNAs were generated by self-splicing using a method described herein. Purified circular RNAs (0.025 picomoles) were transfected into A549 cells (10,000 cells per well in a 96 well plate in media) using LIPOFECTAMINE® MessengerMAX transfection reagent (Invitrogen) according to manufacturer's instructions. MessengerMax alone was used as a control (Carrier only).

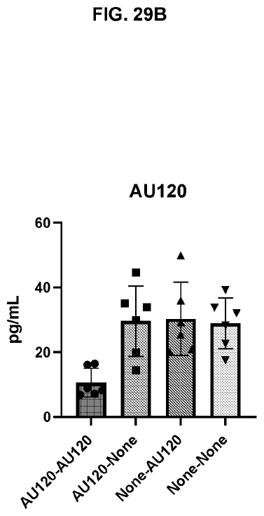

To measure expression of circular RNA encoding GFPd2, the cells were monitored for 64 hours using a live-cell analysis system (Incucyte® instrument, Sartorius). Data were analyzed to have total area under the curve (AUC) and Cmax as an output. The results of this experiment are shown in FIG. 29A as the total AUC.

### Experiment 2: Polypeptide B-HiBIT

Circular RNAs were generated by self-splicing using a method described herein. Purified circular RNAs (0.025 picomoles) were transfected into HEK293T cells (10,000 cells per well in a 96 well plate in serum-free media) using LIPOFECTAMINE® MessengerMAX transfection reagent (Invitrogen) according to manufacturer's instructions.

To measure expression of circular RNA encoding a polypeptide B-HiBiT, polypeptide B-HiBiT levels were measured at 24 hours post-transfection by measuring luminescence from the HiBiT tag, using a bioluminescent protein detection assay following manufacturer's instructions (Nano-Glo® HiBIT Extracellular Detection System, Promega Catalog No. N420). The results of this experiment are shown in FIG. 29B.

### Example 24: Effect of Dual Spacer Designs on Polypeptide Expression from Circular RNA

This example demonstrates the effect of dual spacer designs with (i) identical lengths and sequences or (ii) different lengths and sequences on expression of ORFs from circular RNAs in mammalian cells.

In this example, DNA constructs were designed to include an IRES, an ORF, and a 5′ spacer element (“single spacer design”), or both a 5′ spacer element and a 3′ spacer element (“dual spacer design”). DNA constructs included a modified CVB3 IRES, a nucleotide sequence encoding GFPd2 or a nucleotide sequence encoding polypeptide B having a HiBiT peptide tag as the ORF, and spacer elements as shown in Table 40.

TABLE 40
Spacer elements

| 5′ spacer | 3′ spacer |
|---|---|
| element | element |
| AU120 | AC50 |
| AC50 | AU120 |
| AC50 | AC50 |
| AC50 | None |
| None | AC50 |
| AU120 | AC120 |
| AC120 | AC120 |
| AC120 | None |
| None | AC120 |
| None | None |

### Experiment 1: GFPd2

Circular RNAs were generated by self-splicing using a method described herein. Purified circular RNAs (0.025 picomoles) were transfected into A549 cells (10,000 cells per well in a 96 well plate in media) using LIPOFECTAMINE® MessengerMAX transfection reagent (Invitrogen) according to manufacturer's instructions. MessengerMax alone was used as a control (Carrier only).

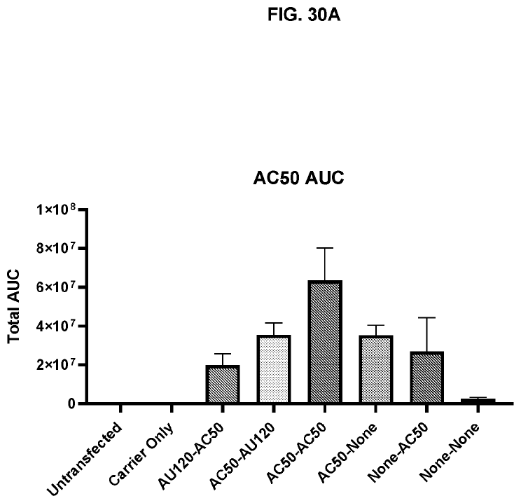

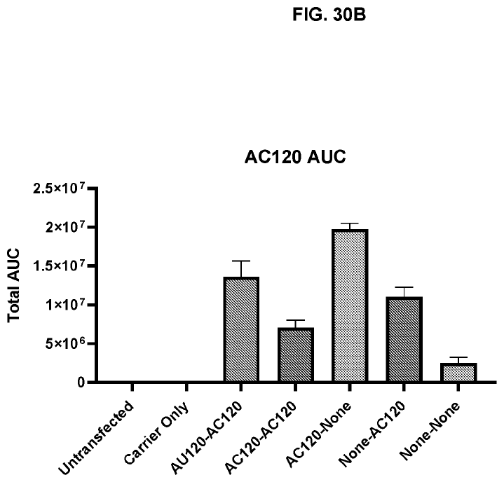

To measure expression of circular RNA encoding GFPd2, the cells were monitored for 64 hours using a live-cell analysis system (Incucyte® instrument, Sartorius). Data were analyzed to have total area under the curve (AUC) and Cmax as an output. The results of this experiment are shown in FIG. 30A (AC50) and FIG. 30B (AC120) as the total AUC.

### Experiment 2: Polypeptide B-HiBIT

Circular RNAs were generated by self-splicing using a method described herein. Purified circular RNAs (0.025 picomoles) were transfected into HEK293T cells (10,000 cells per well in a 96 well plate in serum-free media) using LIPOFECTAMINE® MessengerMAX transfection reagent (Invitrogen) according to manufacturer's instructions.

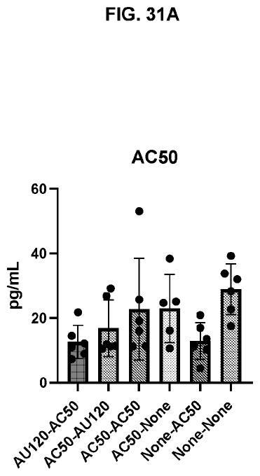

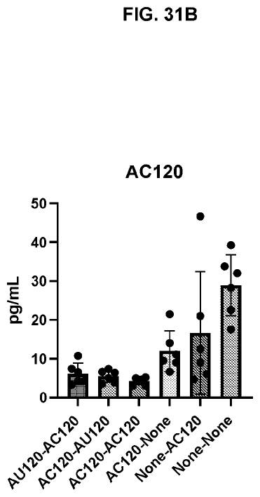

To measure expression of circular RNA encoding a polypeptide B-HiBiT, polypeptide B-HiBIT levels were measured at 24 hours post-transfection by measuring luminescence from the HiBiT tag, using a bioluminescent protein detection assay following manufacturer's instructions (Nano-Glo® HiBIT Extracellular Detection System, Promega Catalog No. N420). The results of this experiment are shown in FIG. 31A (AC50) and FIG. 31B (AC120).

### Example 25: Effect of Spacers and Translation Enhancer on Polypeptide Expression from Circular RNAs

This example demonstrates the effect of spacers and translation enhancers on expression of ORFs from circular RNAs in mammalian cells.

Circular RNAs were designed to include an IRES and an ORF. The following DNA constructs were designed and included from 5′ to 3′; and
(i) a 5′ spacer element, an IRES, an ORF, and a 3′ translation enhancer;
(ii) a 5′ translation enhancer, an IRES, an ORF, and a 3′ translation enhancer;
(iii) a 5′ translation enhancer, an IRES, and ORF, and a 3′ translation enhancer;
(iv) a 5′ translation enhancer, an IRES, and an ORF;
(v) an IRES, an ORF, and a 3′ translation enhancer;
(vi) an IRES and an ORF.

For Experiment 1, the DNA constructs included a modified CVB3 IRES, a nucleotide sequence encoding GFPd2 as the ORF, and spacer elements with a motif as shown in Table 41. For Experiment 2, the DNA constructs included a modified CVB3 IRES, a nucleotide sequence encoding polypeptide B having a HiBiT peptide tag, and spacer elements and/or translation enhancers with a motif as shown in Table 41.

TABLE 41
Spacer Elements

| 5′ spacer element | 3′ spacer element |
|---|---|
| or 5′ translation | or 3′ translation |
| enhancer | enhancer |
| AU120 | FcIgG |
| FcIgG | AU120 |
| FcIgG | FcIgG |
| FcIgG | None |
| None | FcIgG |
| AU120 | TP53I3 |
| TP53I3 | AU120 |
| TP53I3 | TP53I3 |
| TP53I3 | None |
| None | TP53I3 |
| AU120 | LPS1 |
| LPS1 | AU120 |
| LPS1 | LPS1 |
| LPS1 | None |
| None | LPS1 |
| AU120 | Histone4E |
| Histone4E | AU120 |
| Histone4E | Histone4E |
| Histone4E | None |
| None | Histone4E |
| AU120 | AU120 |
| AU120 | None |
| None | AU120 |
| None | None |

### Experiment 1: GFPd2

Circular RNAs were generated by self-splicing using a method described herein. Purified circular RNAs (0.025 picomoles) were transfected into A549 cells (10,000 cells per well in a 96 well plate in media) using LIPOFECTAMINE® MessengerMAX transfection reagent (Invitrogen) according to manufacturer's instructions. MessengerMax alone was used as a control (Carrier only).

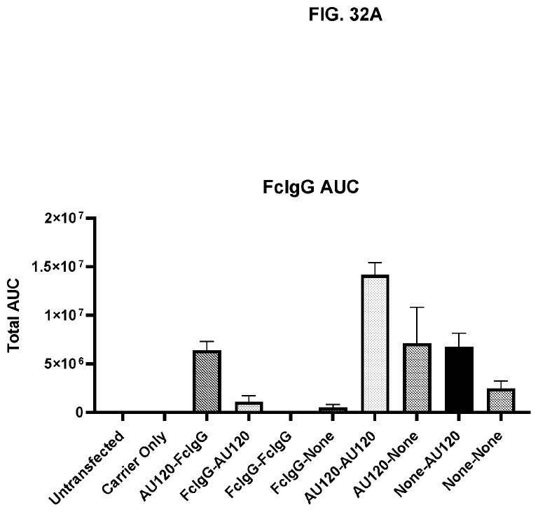

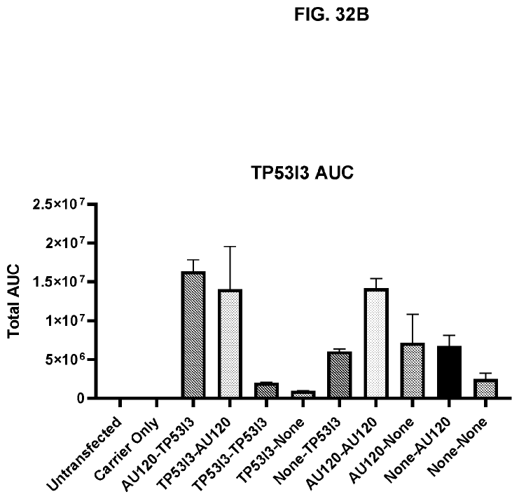

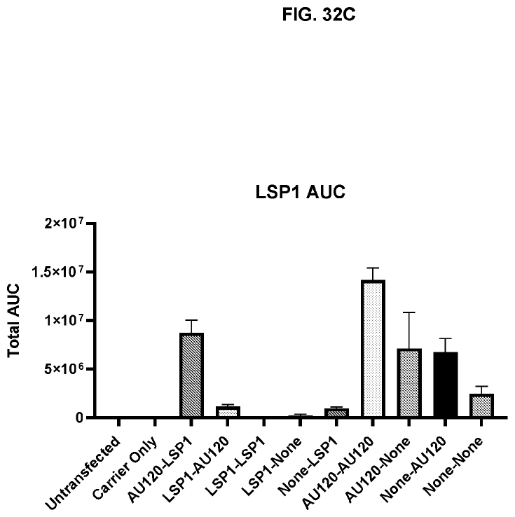

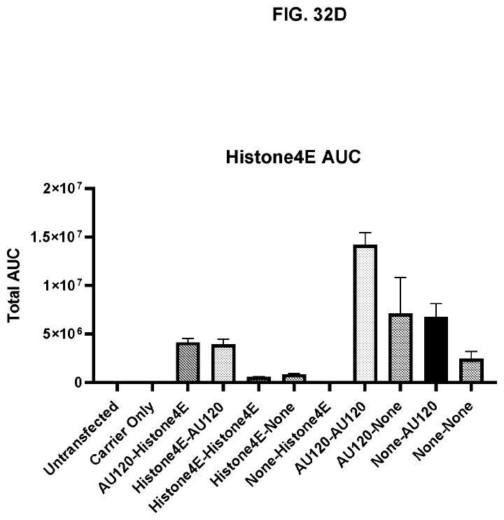

To measure expression of circular RNA encoding GFPd2, the cells were monitored for 64 hours using a live-cell analysis system (Incucyte® instrument, Sartorius). Data were analyzed to have total area under the curve (AUC) and Cmax as an output. FIGS. 32A-32D show the total AUC for FcIgG (FIG. 32A), TP5313 (FIG. 32B), LSP1 (FIG. 32C), and Histone4E (FIG. 32D).

### Experiment 2: Polypeptide B-HiBIT

Circular RNAs were generated by self-splicing using a method described herein. Purified circular RNAs (0.025 picomoles) were transfected into HEK293T cells (10,000 cells per well in a 96 well plate in serum-free media) using LIPOFECTAMINE® MessengerMAX transfection reagent (Invitrogen) according to manufacturer's instructions.

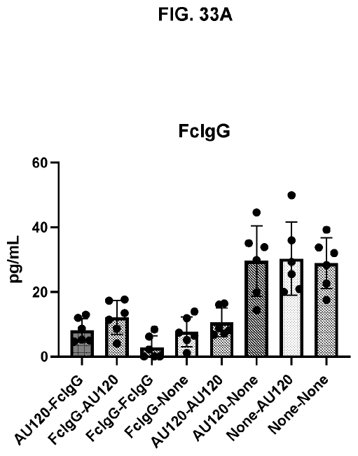

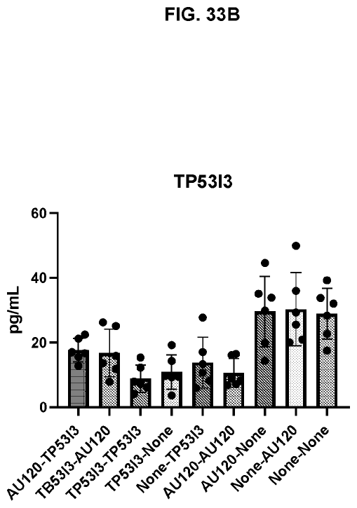

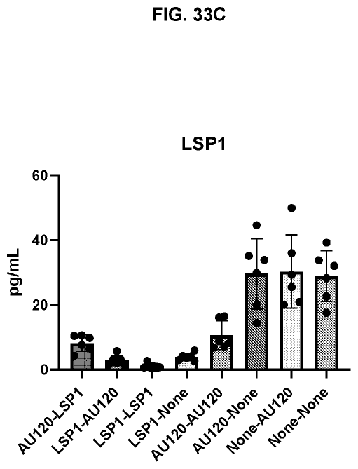

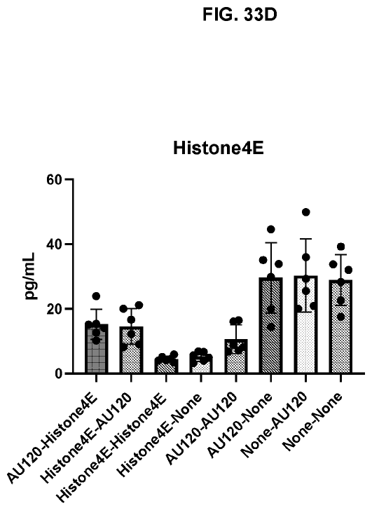

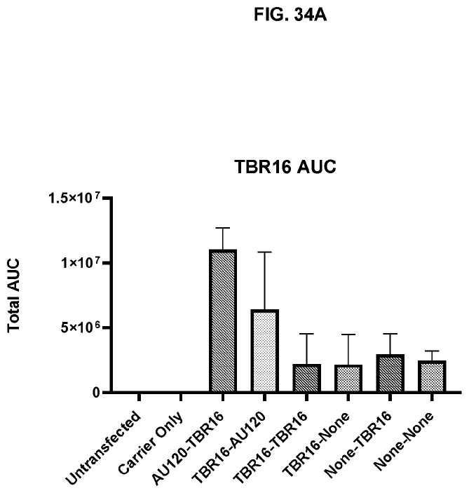

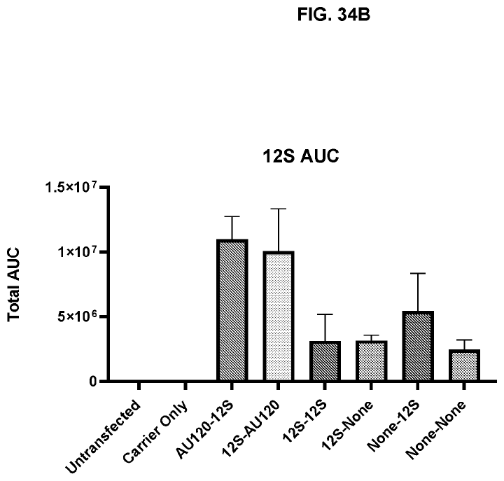

To measure expression of circular RNA encoding a polypeptide B-HiBiT, polypeptide B-HiBIT levels were measured at 24 hours post-transfection by measuring luminescence from the HiBiT tag, using a bioluminescent protein detection assay following manufacturer's instructions (Nano-Glo® HiBIT Extracellular Detection System, Promega Catalog No. N420). The results of this experiment are shown in FIG. 33A (FcIgG), FIG. 33B (TP5313), FIG. 33C (LSP1), and FIG. 33D (Histone4E).

### Example 26: Effect of Spacer Elements and Translation Enhancers on Polypeptide Expression from Circular RNAs

This example demonstrates the effect of spacer elements and translation enhancers on expression of polypeptides from circular polyribonucleotides in mammalian cells.

Circular RNAs were designed to include an IRES and an ORF. The following DNA constructs were designed and included from 5′ to 3′:
(i) a 5′ spacer element, an IRES, an ORF, and a 3′ translation enhancer;
(ii) a 5′ translation enhancer, an IRES, an ORF, and a 3′ spacer element;
(iii) a 5′ translation enhancer, an IRES, an ORF, and a 3′ translation enhancer;
(iv) a 5′ translation enhancer, an IRES, and an ORF;
(v) an IRES, an ORF, and a 3′ translation enhancer;
(vi) an IRES and an ORF

For Experiment 1, the DNA constructs included a modified CVB3 IRES, a nucleotide sequence encoding GFPd2 as the ORF, a polyAT sequence of 120 nucleotides in length as the spacer element(s), and a translation enhancer with a motif as shown in Table 42. For Experiment 2, the DNA constructs included a modified CVB3 IRES, a nucleotide sequence encoding polypeptide B having a HiBiT peptide tag, a polyAT sequence of 120 nucleotides in length as the spacer element(s), and a translation enhancer with a motif as shown in Table 42.

TABLE 42
Translation Enhancers

| Translation | SEQ |
|---|---|
| Enhancer | ID NO |
| TBR16 | 90 |
| 12S | 178 |
| TRAM1 | 163 |
| GPX4 | 304 |

### Experiment 1: GFPd2

Circular RNAs were generated by self-splicing using a method described herein. Purified circular RNAs (0.025 picomoles) were transfected into A549 cells (10,000 cells per well in a 96 well plate in media) using LIPOFECTAMINE® MessengerMAX transfection reagent (Invitrogen) according to manufacturer's instructions. MessengerMax alone was used as a control (Carrier only).

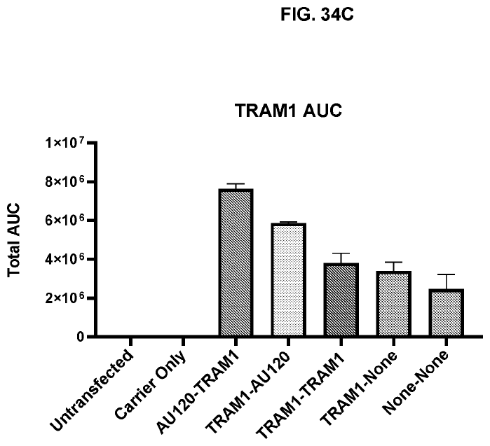

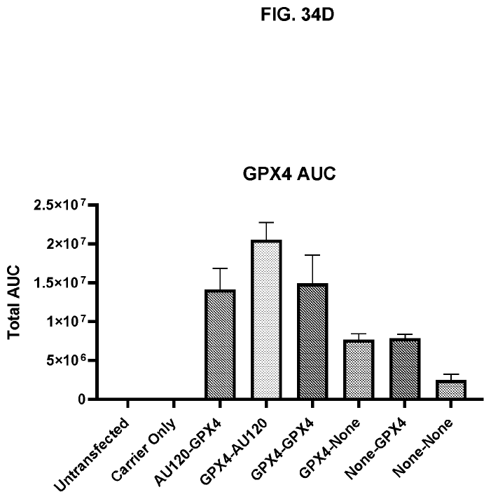

To measure expression of circular RNA encoding GFPd2, the cells were monitored for 64 hours using a live-cell analysis system (Incucyte® instrument, Sartorius). Data were analyzed to have total area under the curve (AUC) and Cmax as an output. FIGS. 34A-34D show the total AUC for TBR16 (FIG. 34A), 12S (FIG. 34B), TRAM1 (FIG. 34C), and GPX4 (FIG. 34D).

### Experiment 2: Polypeptide B-HiBIT

Circular RNAs were generated by self-splicing using a method described herein. Purified circular RNAs (0.025 picomoles) were transfected into HEK293T cells (10,000 cells per well in a 96 well plate in serum-free media) using LIPOFECTAMINE® MessengerMAX transfection reagent (Invitrogen) according to manufacturer's instructions.

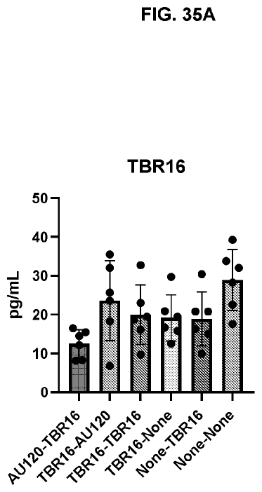

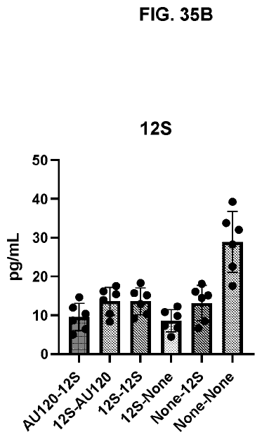

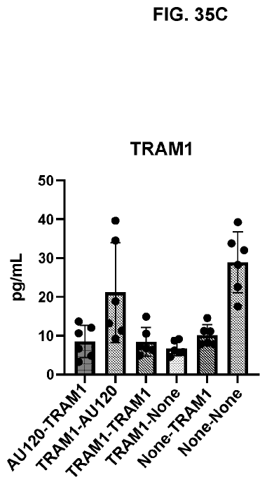

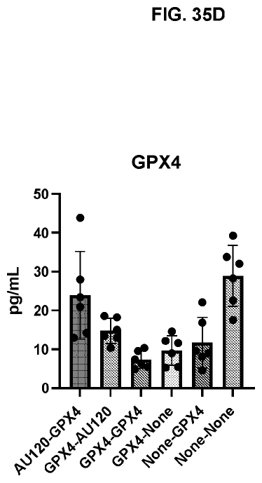

To measure expression of circular RNA encoding a polypeptide B-HiBiT, polypeptide B-HiBiT levels were measured at 24 hours post-transfection by measuring luminescence from the HiBiT tag, using a bioluminescent protein detection assay following manufacturer's instructions (Nano-Glo® HiBIT Extracellular Detection System, Promega Catalog No. N420). The results of this experiment are shown in FIG. 35A (TBR16), FIG. 35B (12S), FIG. 35C (TRAM1), and FIG. 35D (GPX4).

### Other Embodiments

All publications, patents, and patent applications mentioned in the above specification are hereby incorporated by reference to the same extent as if each individual publication, patent or patent application was specifically and individually indicated to be incorporated by reference in its entirety. Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features set forth herein.

### NUMBERED EMBODIMENTS

[1] A circular polyribonucleotide comprising, in the following order from 5′ to 3′:
(a) a first post-circularization element;
(b) one or more expression augmenting element;
(c) a polyribonucleotide cargo; and
(d) a second post-circularization element;
wherein the first post-circularization element and the second post-circularization element together form a circularization junction.
[2] A circular polyribonucleotide comprising, in the following order from 5′ to 3′:
   (a) a first post-circularization element;
   (b) a polyribonucleotide cargo;
   (c) one or more expression augmenting element; and
   (d) a second post-circularization element;
   wherein the first post-circularization element and the second post-circularization element together form a circularization junction.
[3] The circular polyribonucleotide of paragraph [1] or [2], wherein the one or more expression augmenting element comprises a fusion of a translation enhancer and a spacer element comprising a polyA region comprising a polyA region.
[4] The circular polyribonucleotide of any one of paragraphs [1]-[3], wherein the one or more expression augmenting element comprises a translation enhancer.
[5] The circular polyribonucleotide of paragraph [3] or [4], wherein the translation enhancer is from a plant virus.
[6] The circular polyribonucleotide of paragraph [3] or [4], wherein the translation enhancer is from a mammalian virus.
[7] The circular polyribonucleotide of paragraph [3] or [4], wherein the translation enhancer is selected from the group comprising a BYDV like-element (BTE), a translation enhancer element (TED), a PMV/PEMV-like translation enhancer (PTE), an I-shaped structure (ISS), a Y-shaped structure (YSS), a t-shaped structure (TSS), dumbbell shaped structure, viral RNA UTRs (including Dengue, West Nile, Zika, Rotavirus), EMCV, CVB3, hepatitis B virus posttranscriptional regulatory element, human genomic fragments, a histone mRNA sequence, a cyclin D mRNA sequence, or an elF4 g aptamer sequence.
[8] The circular polyribonucleotide of paragraph [3] or [4], wherein the translation enhancer is TBR16, 12S, TRAM1, or GPX4.
[9] The circular polyribonucleotide of any one of paragraphs [3]-[8], wherein the translation enhancer comprises between 50 and 2000 ribonucleotides.
[10] The circular polyribonucleotide of paragraph [9], wherein the translation enhancer comprises between 60 and 800 ribonucleotides.
[11] The circular polyribonucleotide of any one of paragraphs [3]-[10], wherein the circular polyribonucleotide exhibits at least 2-fold greater stability in comparison to a polyribonucleotide without a translation enhancer.
[12] The circular polyribonucleotide of any one of paragraphs [3]-[11], wherein the circular polyribonucleotide may be detected for at least 2-fold longer in comparison to a polyribonucleotide without a translation enhancer after the circular polyribonucleotide is administered to a subject.
[13] The circular polyribonucleotide of any one of paragraphs [3]-[12], wherein the circular polyribonucleotide comprises a polynucleotide cargo encodes a polypeptide, wherein the polypeptide has at least 4-fold greater expression in comparison to a polyribonucleotide without a translation enhancer.
[14] The circular polyribonucleotide of any one of paragraphs [3]-[13], wherein the expression augmenting element comprises a stability element.
[15] The circular polyribonucleotide of paragraph [14], wherein the stability element is a 3′ UTR.
[16] The circular polyribonucleotide of paragraph [15], wherein the 3′ UTR is from a gene encoding a translocation associated membrane protein 1 (TRAM1), a transmembrane p24 trafficking protein 2 (TMED2), a vesicle associated membrane protein 3 (VAMP3), a CXXC repeat containing interactor of PDZ3 domain (CRIP), an adaptor related protein complex 2 subunit alpha 2 (AP2A2), a proteasome 26S subunit, a non-ATPase 5 (PSMD5), a glutathione peroxidase 4 (GPX4), or a protein kinase AMP-activated non-catalytic subunit beta 1 (PRKAB1).
[17] The circular polyribonucleotide of paragraph [16], wherein the 3′ UTR is a 3′UTR from a human beta actin, DDB2, TP5313, FcIgG, LSP1, AES, DRB4, or a mitochondrially encoded 12S rRNA.
[18] The circular polyribonucleotide of any one of paragraphs [14]-[17], wherein the stability element comprises between 50 and 2000 ribonucleotides.
[19] The circular polyribonucleotide of paragraph [18], wherein the stability element comprises between 60 and 800 ribonucleotides.
[20] The circular polyribonucleotide of any one of paragraphs [14]-[19], wherein the circular polyribonucleotide exhibits at least 2-fold greater stability in comparison to a polyribonucleotide without a stability enhancer.
[21] The circular polyribonucleotide of any one of paragraphs [14]-[20], wherein the circular polyribonucleotide may be detected for at least 2-fold longer in comparison to a polyribonucleotide without a stability element after the circular polyribonucleotide is administered to a subject.
[22] The circular polyribonucleotide of any one of paragraphs [14]-[21], wherein the polynucleotide cargo encodes a polypeptide, wherein the polypeptide has at least 2-fold greater expression in comparison to a polyribonucleotide without a stability element.
[23] The circular polyribonucleotide of any one of paragraphs [1]-[22], wherein the circular polyribonucleotide further comprises one or more spacer elements comprising a polyA region.
[24] The circular polyribonucleotides of paragraph [23], wherein the spacer element has a length of between 100 to 500 ribonucleotides.
[25] The circular polyribonucleotide of paragraph [24], wherein the spacer element has a length of between 120 to 500 ribonucleotides.
[26] The circular polyribonucleotide of paragraph [25], wherein the spacer element has a length of between 100 to 300 ribonucleotides.
[27] A circular polyribonucleotide comprising, in the following order from 5′ to 3′:
   (a) a first post-circularization element;
   (b) a first expression augmenting element
   (c) a polyribonucleotide cargo;
   (d) a second expression augmenting element; and a second post-circularization element;
wherein the first post-circularization element and the second post-circularization element together form a circularization junction.
[28] The circular polyribonucleotide of paragraph [27], wherein the first expression augmenting element comprises a fusion of a translation enhancer and a spacer element.
[29] The circular polyribonucleotide of paragraph [27], wherein the second expression augmenting element comprises a fusion of a translation enhancer and a spacer element.
[30] The circular polyribonucleotide of paragraph or [29], wherein the first expression augmenting element comprises a translation enhancer.
[31] The circular polyribonucleotide of any one of paragraphs [27], [28], or [30], wherein the second expression augmenting element comprises a translation enhancer.
[32] The circular polyribonucleotide of any one of paragraphs [28]-[31], wherein the translation enhancer is from a plant virus.
[33] The circular polyribonucleotide of any one of paragraphs [28]-[32], wherein the translation enhancer is from a mammalian virus.
[34] The circular polyribonucleotide of any one of [28]-[33], wherein the translation enhancer is selected from the group comprising a BTE, a TED, a PTE, an ISS, a YSS, a TSS, dumbbell shaped structure, viral RNA UTRs (including Dengue, West Nile, Zika, Rotavirus), EMCV, CVB3, hepatitis B virus posttranscriptional regulatory element, human genomic fragments, a histone mRNA sequence, a cyclin D mRNA sequence, or an elF4 g aptamer sequence.
[35] The circular polyribonucleotide of any one of paragraphs [28]-[34], wherein the translation enhancer is TBR16, 12S, TRAM1, or GPX4.
[36] The circular polyribonucleotide of any one of paragraphs [28]-[35], wherein the translation enhancer comprises between 50 and 2000 ribonucleotides.
[37] The circular polyribonucleotide of paragraph [36], wherein the translation enhancer comprises between 60 and 800 ribonucleotides.
[38] The circular polyribonucleotide of any one of paragraphs [28]-[37], wherein the circular polyribonucleotide exhibits at least 2-fold greater stability in comparison to a polyribonucleotide without a translation enhancer.
[39] The circular polyribonucleotide of any one of paragraphs [28]-[38], wherein the circular polyribonucleotide may be detected for at least 2-fold longer in comparison to a polyribonucleotide without a translation enhancer after the circular polyribonucleotide is administered to a subject.
[40] The circular polyribonucleotide of any one of paragraphs [28]-[39], wherein the circular polyribonucleotide is polynucleotide cargo encodes a polypeptide, wherein the polypeptide has at least 4-fold greater expression in comparison to a polyribonucleotide without a translation enhancer.
[41] The circular polyribonucleotide of any one of paragraphs [27], [29], or [31]-[40], wherein the first expression augmenting element comprises a stability element.
[42] The circular polyribonucleotide of any one of paragraphs [27], [28], [30], or [32]-[41], wherein the second expression augmenting element comprises a stability element.
[43] The circular polyribonucleotide of paragraph or [42], wherein the stability element is a 3′ UTR.
[44] The circular polyribonucleotide of paragraph [43], wherein the 3′ UTR is from a gene encoding a TRAM1, a TMED2, a VAMP3, a CRIP, an AP2A2, a proteasome 26S subunit, a PSMD5, a GPX4, or a PRKAB1.
[45] The circular polyribonucleotide of paragraph [43], wherein the 3′ UTR is a 3′UTR from a human beta actin, DDB2, TP5313, FcIgG, LSP1, AES, DRB4, or a mitochondrially encoded 12S rRNA.
[46] The circular polyribonucleotide of any one of paragraphs [41]-[45], wherein the stability element comprises between 50 and 2000 ribonucleotides.
[47] The circular polyribonucleotide of paragraph [46], wherein the stability element comprises between 60 and 800 ribonucleotides.
[48] The circular polyribonucleotide of any one of paragraphs [41]-[47], wherein the circular polyribonucleotide exhibits at least 2-fold greater stability in comparison to a polyribonucleotide without a stability enhancer.
[49] The circular polyribonucleotide of any one of paragraphs [41]-[48], wherein the circular polyribonucleotide may be detected for at least 2-fold longer in comparison to a polyribonucleotide without a stability element after the circular polyribonucleotide is administered to a subject.
[50] The circular polyribonucleotide of any one of paragraphs [41]-[49], wherein the polynucleotide cargo encodes a polypeptide, wherein the polypeptide has at least 2-fold greater expression in comparison to a polyribonucleotide without a stability element.
[51] The circular polyribonucleotide of any one of paragraphs [27]-[50], wherein the circular polyribonucleotide further comprises one or more spacer elements comprising a polyA region.
[52] The circular polyribonucleotides of paragraph [51], wherein the spacer element has a length of between 100 to 500 ribonucleotides.
[53] The circular polyribonucleotide of paragraph [52], wherein the spacer element has a length of between 120 to 500 ribonucleotides.
[54] The circular polyribonucleotide of paragraph [53], wherein the spacer element has a length of between 100 to 300 ribonucleotides.
[55] A circular polyribonucleotide comprising, in the following order from 5′ to 3′:
   (a) a first post-circularization element;
   (b) a spacer element
   (c) a polyribonucleotide cargo;
   (d) an expression augmenting element and
   (e) a second post-circularization element
   and wherein the first post-circularization element and the second post-circularization element together form a circularization junction.
[56] A circular polyribonucleotide comprising, in the following order from 5′ to 3′:
   (a) a first post-circularization element;
   (b) an expression augmenting element;
   (c) a polyribonucleotide cargo;
   (d) a spacer element; and
   (e) a second post-circularization element
   and wherein the first post-circularization element and the second post-circularization element together form a circularization junction.
[57] A circular polyribonucleotide comprising, in the following order from 5′ to 3′:
   (a) a first post-circularization element;
   (b) a first expression augmenting element;
   (c) a polyribonucleotide cargo;
   (d) a second expression augmenting element; and
   (e) a second post-circularization element
   and wherein the first post-circularization element and the second post-circularization element together form a circularization junction.
[58] A circular polyribonucleotide comprising, in the following order from 5′ to 3′:
   (a) a first post-circularization element;
   (b) a first spacer element having a length of at least 100 ribonucleotides;
   (c) a polyribonucleotide cargo;
   (d) a second spacer element; and
   (e) a second post-circularization element;
   and wherein the first post-circularization element and the second post-circularization element together form a circularization junction.
[59] The circular polyribonucleotides of paragraph [58], wherein the first spacer element has a length of between 100 to 500 ribonucleotides.
[60] The circular polyribonucleotides of paragraph [59], wherein the second spacer element has a length of between 100 to 500 ribonucleotides.
[61] The circular polyribonucleotide of paragraph [60], wherein the first spacer element and the second spacer element each comprise 120 to 500 ribonucleotides.
[62] The circular polyribonucleotide of paragraph [61], wherein the first spacer element and the second spacer element each have a length of 150 to 500 ribonucleotides.
[63] The circular polyribonucleotide of paragraph [62], wherein the first spacer element and the second spacer element each have a length of 200 to 500 ribonucleotides.
[64] The circular polyribonucleotide of paragraph [63], wherein the first spacer element and the second spacer element each have a length of 100 to 300 ribonucleotides.
[65] The circular polyribonucleotide of paragraph [64], wherein the first spacer element and the second spacer element each have a length of 100 to 200 ribonucleotides.
[66] The circular polyribonucleotide of paragraph [65], wherein the first spacer element and the second spacer element each have a length of 120 to 300 ribonucleotides.
[67] The circular polyribonucleotide of paragraph [66], wherein the first spacer element and the second spacer element each have a length of 150 to 300 ribonucleotides.
[68] The circular polyribonucleotides of any one of paragraphs [59]-[67], wherein the difference between the length of the first spacer element and the length of the second spacer element is 0 to 100 nucleotides.
[69] The circular polyribonucleotides of paragraph [68], wherein the difference between the length of the first spacer element and the length of the second spacer element is 0 to 50 nucleotides.
[70] The circular polyribonucleotide of any one of paragraphs [59]-[69], wherein the first spacer or the second spacer element consists of:
   (i) a polyA region comprising 80% to 100% adenosine residues;
   (ii) a polyAC region comprising between 80% to 100% adenosine or cytosine residues;
   (iii) a polyAU region comprising between 80% to 100% adenosine or uridine residues; or
   (iv) a polyAG region comprising between 80% to 100% adenosine or guanosine residues.
[71] The circular polyribonucleotide of any one of paragraphs [59]-[70], wherein the circular polyribonucleotide exhibits at least 2-fold greater stability in comparison to a polyribonucleotide a first spacer element and a second spacer element.
[72] The circular polyribonucleotide of any one of paragraphs [59]-[71], wherein the circular polyribonucleotide may be detected for at least 2-fold longer in comparison to a polyribonucleotide without a first spacer element and a second spacer element after the circular polyribonucleotide is administered to a subject.
[73] The circular polyribonucleotide of any one of paragraphs [59]-[72], wherein the polynucleotide cargo encodes a polypeptide, wherein the polypeptide has at least 4-fold greater expression in comparison to a polyribonucleotide without the first spacer element and the second spacer element.
[74] The circular polyribonucleotide any one of paragraphs [1]-[73], wherein the polyribonucleotide cargo comprises an expression sequence.
[75] The circular polyribonucleotide of paragraph [74], wherein the polyribonucleotide cargo comprises an IRES operably linked an expression sequence.
[76] The circular polyribonucleotide of paragraph or [75], wherein the expression sequence further comprises a 3′ untranslated region or a 5′ untranslated region.
[77] The circular polyribonucleotide of any one of paragraphs [74]-[76], wherein the expression sequence encodes a polypeptide.
[78] The circular polyribonucleotide of any one of paragraphs [1]-[77], wherein the polyribonucleotide comprises between 500 and 20,000 ribonucleotides.
[79] The circular polyribonucleotide of paragraph [78], wherein the polyribonucleotide comprises between 2,000 and 20,000 ribonucleotides.
[80] The circular polyribonucleotide of any one of paragraphs [1]-[79], wherein the circularization junction comprises a splice junction.
[81] The circular polyribonucleotide of paragraph [80], wherein the first post-circularization element comprises a first exon fragment and the second post-circularization element comprises a second exon fragment, and wherein the first exon fragment and the second exon fragment are joined by the splice junction.
[82] The circular polyribonucleotide of any one of paragraphs [1]-[81], wherein the circularization junction comprises an oligonucleotide splint that is hybridized to the first post-circularization element and to the second post-circularization element.
[83] The circular polyribonucleotide of paragraph [82], wherein the oligonucleotide splint is a DNA splint or an RNA splint.
[84] The circular polyribonucleotide of paragraph or [83], wherein the first post-circularization element comprises a region that is capable of hybridizing to a first region of the oligonucleotide splint and the second post-circularization element comprises a region that is capable of hybridizing to a second region of the oligonucleotide splint.
[85] A linear polyribonucleotide comprising, in the following order from 5′ to 3′:
   (a) a first post-circularization element;
   (b) one or more expression augmenting element;
   (c) a polyribonucleotide cargo; and
   (d) a second post-circularization element;
   wherein the first post-circularization element and the second post-circularization element together form a circularization junction.
[86] A linear polyribonucleotide comprising, in the following order from 5′ to 3′:
   (a) a first post-circularization element;
   (b) a polyribonucleotide cargo;
   (c) one or more expression augmenting element; and
   (d) a second post-circularization element;
   wherein the first post-circularization element and the second post-circularization element together form a circularization junction.
[87] The linear polyribonucleotide of paragraph or [86], wherein the one or more expression augmenting element comprises a fusion of a translation enhancer and a spacer element comprising a polyA region.
[88] The linear polyribonucleotide of any one of paragraphs [85]-[87], wherein the one or more expression augmenting element comprises a translation enhancer.
[89] The linear polyribonucleotide of paragraph or [88], wherein the translation enhancer is from a plant virus.
[90] The linear polyribonucleotide of paragraph or [88], wherein the translation enhancer is from a mammalian virus.
[91] The linear polyribonucleotide of paragraph or [88], wherein the translation enhancer is selected from the group comprising a BTE, a TED, a PTE, an ISS, a YSS, a TSS, dumbbell shaped structure, viral RNA UTRs (including Dengue, West Nile, Zika, Rotavirus), EMCV, CVB3, hepatitis B virus posttranscriptional regulatory element, human genomic fragments, a histone mRNA sequence, a cyclin D mRNA sequence, or an elF4 g aptamer sequence.
[92] The linear polyribonucleotide of paragraph or [88], wherein the translation enhancer is TBR16, 12S, TRAM1, or GPX4.
[93] The linear polyribonucleotide of any one of paragraphs [87]-[92], wherein the translation enhancer comprises between 50 and 2000 ribonucleotides.
[94] The linear polyribonucleotide of paragraph [93], wherein the translation enhancer comprises between 60 and 800 ribonucleotides.
[95] The linear polyribonucleotide of any one of paragraphs [87]-[94], wherein the linear polyribonucleotide exhibits at least 2-fold greater stability in comparison to a polyribonucleotide without a translation enhancer.
[96] The linear polyribonucleotide of any one of paragraphs [87]-[95], wherein the linear polyribonucleotide may be detected for at least 2-fold longer in comparison to a polyribonucleotide without a translation enhancer after the linear polyribonucleotide is administered to a subject.
[97] The linear polyribonucleotide of any one of paragraphs [87]-[96], wherein the linear polyribonucleotide is polynucleotide cargo encodes a polypeptide, wherein the polypeptide has at least 4-fold greater expression in comparison to a polyribonucleotide without a translation enhancer.
[98] The linear polyribonucleotide of any one of paragraphs [87]-[97], wherein the expression augmenting element comprises a stability element.
[99] The linear polyribonucleotide of paragraph [98], wherein the stability element is a 3′ UTR.
[100] The linear polyribonucleotide of paragraph [99], wherein the 3′ UTR is from a gene encoding a TRAM1, a TMED2, a VAMP3, a CRIP, an AP2A2, a proteasome 26S subunit, a PSMD5, a GPX4, or a PRKAB1.
[101] The linear polyribonucleotide of paragraph [99], wherein the 3′ UTR is a 3′UTR from a human beta actin, DDB2, TP5313, FcIgG, LSP1, AES, DRB4, or a mitochondrially encoded 12S rRNA.
[102] The linear polyribonucleotide of any one of paragraphs [98]-[101], wherein the stability element comprises between 50 and 2000 ribonucleotides.
[103] The linear polyribonucleotide of paragraph [102], wherein the stability element comprises between 60 and 800 ribonucleotides.
[104] The linear polyribonucleotide of any one of paragraphs [98]-[103], wherein the linear polyribonucleotide exhibits at least 2-fold greater stability in comparison to a polyribonucleotide without a stability enhancer.
[105] The linear polyribonucleotide of any one of paragraphs [98]-[104], wherein the linear polyribonucleotide may be detected for at least 2-fold longer in comparison to a polyribonucleotide without a stability element after the linear polyribonucleotide is administered to a subject.
[106] The linear polyribonucleotide of any one of paragraphs [98]-[105], wherein the polynucleotide cargo encodes a polypeptide, wherein the polypeptide has at least 2-fold greater expression in comparison to a polyribonucleotide without a stability element.
[107] The linear polyribonucleotide of any one of paragraphs [85]-[106], wherein the linear polyribonucleotide further comprises one or more spacer elements.
[108] The linear polyribonucleotides of paragraph [107], wherein the spacer element has a length of between 100 to 500 ribonucleotides.
[109] The linear polyribonucleotide of paragraph [108], wherein the spacer element has a length of between 120 to 500 ribonucleotides.
[110] The linear polyribonucleotide of paragraph [109], wherein the spacer element has a length of between 100 to 300 ribonucleotides.
[111] A linear polyribonucleotide comprising, in the following order from 5′ to 3′:
   (a) a first post-circularization element;
   (b) a first expression augmenting element
   (c) a polyribonucleotide cargo;
   (d) a second expression augmenting element; an € (e) a second post-circularization element;
   wherein the first post-circularization element and the second post-circularization element together form a circularization junction.
[112] The linear polyribonucleotide of paragraph [111], wherein the first expression augmenting element comprises a fusion of a translation enhancer and a spacer element comprising a polyA region.
[113] The linear polyribonucleotide of paragraph or [112], wherein the second expression augmenting element comprises a fusion of a translation enhancer and a spacer element.
[114] The linear polyribonucleotide of paragraph or [113], wherein the first expression augmenting element comprises a translation enhancer.
[115] The linear polyribonucleotide of any one of paragraphs [111], [112], or [114], wherein the second expression augmenting element comprises a translation enhancer.
[116] The linear polyribonucleotide of any one of paragraphs [112]-[115], wherein the translation enhancer is from a plant virus.
[117] The linear polyribonucleotide of any one of paragraphs [112]-[116], wherein the translation enhancer is from a mammalian virus.
[118] The linear polyribonucleotide of any one of [112]-[117], wherein the translation enhancer is selected from the group comprising a BTE, a TED, a PTE, an ISS, a YSS, a TSS, dumbbell shaped structure, viral RNA UTRs (including Dengue, West Nile, Zika, Rotavirus), EMCV, CVB3, hepatitis B virus posttranscriptional regulatory element, human genomic fragments, a histone mRNA sequence, a cyclin D mRNA sequence, or an elF4 g aptamer sequence.
[119] The linear polyribonucleotide of any one of paragraphs [112]-[118], wherein the translation enhancer binds to elF4 g.
[120] The linear polyribonucleotide of any one of paragraphs [112]-[119], wherein the translation enhancer comprises between 50 and 2000 ribonucleotides.
[121] The linear polyribonucleotide of paragraph [120], wherein the translation enhancer comprises between 60 and 800 ribonucleotides.
[122] The linear polyribonucleotide of any one of paragraphs [112]-[121], wherein the linear polyribonucleotide exhibits at least 2-fold greater stability in comparison to a polyribonucleotide without a translation enhancer.
[123] The linear polyribonucleotide of any one of paragraphs [112]-[122], wherein the linear polyribonucleotide may be detected for at least 2-fold longer in comparison to a polyribonucleotide without a translation enhancer after the linear polyribonucleotide is administered to a subject.
[124] The linear polyribonucleotide of any one of paragraphs [112]-[123], wherein the linear polyribonucleotide is polynucleotide cargo encodes a polypeptide, wherein the polypeptide has at least 4-fold greater expression in comparison to a polyribonucleotide without a translation enhancer.
[125] The linear polyribonucleotide of any one of paragraphs [111], [113], or [115]-[124], wherein the first expression augmenting element comprises a stability element.
[126] The linear polyribonucleotide of any one of paragraphs [111], [112], [114], or [116]-[125], wherein the second expression augmenting element comprises a stability element.
[127] The linear polyribonucleotide of paragraph or [126], wherein the stability element is a 3′ UTR.
[128] The linear polyribonucleotide of paragraph [127], wherein the 3′ UTR is from a gene encoding a TRAM1, a TMED2, a VAMP3, a CRIP, an AP2A2, a proteasome 26S subunit, a PSMD5, a GPX4, or a PRKAB1.
[129] The linear polyribonucleotide of paragraph [127], wherein the 3′ UTR is a 3′UTR from a human beta actin, DDB2, TP5313, FcIgG, LSP1, AES, DRB4, or a mitochondrially encoded 12S rRNA.
[130] The linear polyribonucleotide of any one of paragraphs [125]-[129], wherein the stability element comprises between 50 and 2000 ribonucleotides.
[131] The linear polyribonucleotide of paragraph [130], wherein the stability element comprises between 60 and 800 ribonucleotides.
[132] The linear polyribonucleotide of any one of paragraphs [125]-[131], wherein the linear polyribonucleotide exhibits at least 2-fold greater stability in comparison to a polyribonucleotide without a stability enhancer.
[133] The linear polyribonucleotide of any one of paragraphs [125]-[132], wherein the linear polyribonucleotide may be detected for at least 2-fold longer in comparison to a polyribonucleotide without a stability element after the linear polyribonucleotide is administered to a subject.
[134] The linear polyribonucleotide of any one of paragraphs [125]-[133], wherein the polynucleotide cargo encodes a polypeptide, wherein the polypeptide has at least 2-fold greater expression in comparison to a polyribonucleotide without a stability element.
[135] The linear polyribonucleotide of any one of paragraphs [111]-[134], wherein the linear polyribonucleotide further comprises one or more spacer elements.
[136] The linear polyribonucleotides of paragraph [135], wherein the spacer element has a length of between 100 to 500 ribonucleotides.
[137] The linear polyribonucleotide of paragraph [136], wherein the spacer element has a length of between 120 to 500 ribonucleotides.
[138] The linear polyribonucleotide of paragraph [137], wherein the spacer element has a length of between 100 to 300 ribonucleotides.
[139] A linear polyribonucleotide comprising, in the following order from 5′ to 3′:
   (a) a first post-circularization element;
   (b) a spacer element
   (c) a polyribonucleotide cargo;
   (d) an expression augmenting element; and
   (e) a second post-circularization element,
   and wherein the first post-circularization element and the second post-circularization element together form a circularization junction.
[140] A linear polyribonucleotide comprising, in the following order from 5′ to 3′:
   (a) a first post-circularization element;
   (b) an expression augmenting element;
   (c) a polyribonucleotide cargo;
   (d) a spacer element; and
   (e) a second post-circularization element,
   and wherein the first post-circularization element and the second post-circularization element together form a circularization junction.
[141] A linear polyribonucleotide comprising, in the following order from 5′ to 3′:
   (a) a first post-circularization element;
   (b) a first expression augmenting element;
   (c) a polyribonucleotide cargo;
   (d) a second expression augmenting element; and
   (e) a second post-circularization element,
   and wherein the first post-circularization element and the second post-circularization element together form a circularization junction.
[142] A linear polyribonucleotide comprising, in the following order from 5′ to 3′:
   (a) a first post-circularization element;
   (b) a first spacer element having a length of at least 100 ribonucleotides;
   (c) a polyribonucleotide cargo;
   (d) a second spacer element; and
   (e) a second post-circularization element;
   and wherein the first post-circularization element and the second post-circularization element together form a circularization junction.
[143] A linear polyribonucleotide comprising, in the following order from 5′ to 3′:
   (a) a first post-circularization element;
   (b) a first spacer element having a length of at least 100 ribonucleotides;
   (c) a polyribonucleotide cargo;
   (d) a second spacer element and
   (e) a second post-circularization element;
   and wherein the first post-circularization element and the second post-circularization element together form a circularization junction.
[144] The linear polyribonucleotides of paragraph [143], wherein the first spacer element has a length of between 100 to 500 ribonucleotides.
[145] The linear polyribonucleotide of paragraph [144], wherein the first spacer element has a length of between 100 and 300.
[146] The linear polyribonucleotides of paragraph [144], wherein the second spacer element has a length of between 100 to 500 ribonucleotides.
[147] The linear polyribonucleotide of paragraph [146], wherein the first spacer element and the second spacer element each comprise 120 to 500 ribonucleotides.
[148] The linear polyribonucleotide of paragraph [147], wherein the first spacer element and the second spacer element each have a length of 150 to 500 ribonucleotides.
[149] The linear polyribonucleotide of paragraph [148], wherein the first spacer element and the second spacer element each have a length of 200 to 500 ribonucleotides.
[150] The linear polyribonucleotide of paragraph [148], wherein the first spacer element and the second spacer element each have a length of 100 to 300 ribonucleotides.
[151] The linear polyribonucleotide of paragraph [150], wherein the first spacer element and the second spacer element each have a length of 100 to 200 ribonucleotides.
[152] The linear polyribonucleotide of paragraph [150], wherein the first spacer element and the second spacer element each have a length of 120 to 300 ribonucleotides.
[153] The linear polyribonucleotide of paragraph [152], wherein the first spacer element and the second spacer element each have a length of 150 to 300 ribonucleotides.
[154] The linear polyribonucleotides of any one of paragraphs [143]-[153], wherein the difference between the length of the first spacer element and the length of the second spacer element is 0 to 100 nucleotides.
[155] The linear polyribonucleotides of paragraph [154], wherein the difference between the length of the first spacer element and the length of the second spacer element is 0 to 50 nucleotides.
[156] The linear polyribonucleotide of any one of paragraphs [143]-[155], wherein the first spacer or the second spacer element consists of:
   (i) a polyA region comprising 80% to 100% adenosine residues;
   (ii) a polyAC region comprising between 80% to 100% adenosine or cytosine residues;
   (iii) a polyAU region comprising between 80% to 100% adenosine or uridine residues; or
   (iv) a polyAG region comprising between 80% to 100% adenosine or guanosine residues.
[157] The linear polyribonucleotide of any one of paragraphs [143]-[156], wherein the polyribonucleotide exhibits at least 2-fold greater stability in comparison to a polyribonucleotide a first spacer element and a second spacer element.
[158] The linear polyribonucleotide of any one of paragraphs [143]-[156], wherein the polyribonucleotide may be detected for at least 2-fold longer in comparison to a polyribonucleotide without a first spacer element and a second spacer element after the polyribonucleotide is administered to a subject.
[159] The linear polyribonucleotide of any one of paragraphs [143]-[158], wherein the polynucleotide cargo encodes a polypeptide, wherein the polypeptide has at least 4-fold greater expression in comparison to a polyribonucleotide without the first spacer element and the second spacer element.
[160] The linear polyribonucleotide any one of paragraphs [85]-[159], wherein the polyribonucleotide cargo comprises an expression sequence.
[161] The linear polyribonucleotide of paragraph [160], wherein the polyribonucleotide cargo comprises an IRES operably linked an expression sequence.
[162] The linear polyribonucleotide of paragraph or [161], wherein the expression sequence further comprises a 3′ untranslated region or a 5′ untranslated region.
[163] The linear polyribonucleotide of any one of paragraphs [160]-[162], wherein the expression sequence encodes a polypeptide.
[164] The linear polyribonucleotide of any one of paragraphs [85]-[163], wherein the polyribonucleotide comprises between 500 and 20,000 ribonucleotides.
[165] The linear polyribonucleotide of paragraph [164], wherein the polyribonucleotide comprises between 2,000 and 20,000 ribonucleotides.
[166] The linear polyribonucleotide of any one of paragraphs [85]-[165], wherein the circularization junction comprises a splice junction.
[167] The linear polyribonucleotide of paragraph [166], wherein the first post-circularization element comprises a first exon fragment and the second post-circularization element comprises a second exon fragment, and wherein the first exon fragment and the second exon fragment are joined by the splice junction.
[168] The linear polyribonucleotide of any one of paragraphs [85]-[167], wherein the circularization junction comprises an oligonucleotide splint that is hybridized to the first post-circularization element and to the second post-circularization element.
[169] The linear polyribonucleotide of paragraph [168], wherein the oligonucleotide splint is a DNA splint or an RNA splint.
[170] The linear polyribonucleotide of paragraph or [169], wherein the first post-circularization element comprises a region that is capable of hybridizing to a first region of the oligonucleotide splint and the second post-circularization element comprises a region that is capable of hybridizing to a second region of the oligonucleotide splint.
[171] The linear polyribonucleotide of any one of paragraphs [85]-[170], wherein:
   the first circularization element comprises a first catalytic intron fragment, a first splice site dinucleotide, and a first exon fragment; and
   the second circularization element comprises a second catalytic intron fragment, a second splice site dinucleotide, and a second exon fragment.
[172] The linear polyribonucleotide of paragraph [171], wherein the first catalytic intron fragment and the second catalytic intron fragment, together, are capable of self-splicing thereby covalently joining the first exon region and the second exon region to produce a circular polyribonucleotide.
[173] The linear polyribonucleotide of paragraph or [172], wherein the first catalytic intron fragment and the second catalytic intron fragment are from a cyanobacterium Anabaena pre-tRNA-Leu gene or a Tetrahymena pre-rRNA.
[174] The linear polyribonucleotide of any one of paragraphs [85]-[173], wherein the first circularization element comprises a region that is capable of hybridizing to a first region of an oligonucleotide splint and the second circularization element comprises a region that is capable of hybridizing to a second region of the oligonucleotide splint.
[175] A DNA vector comprising an RNA polymerase promoter operably linked to a sequence that encodes the linear polyribonucleotide of any one of paragraphs [85]-[174].
[176] A circular polyribonucleotide produced from the linear polyribonucleotide of any one of paragraphs [85]-[174] or from the DNA vector of paragraph [175].
[177] A pharmaceutical composition comprising the linear polyribonucleotide, the circular polyribonucleotide, or the DNA vector of any one of paragraphs [1]-[174] and a pharmaceutically acceptable excipient.
[178] A method of expressing a polypeptide in a cell or a subject, the method comprising providing to the cell or the subject the linear polyribonucleotide, the circular polyribonucleotide, the DNA vector, or the pharmaceutical composition of one of paragraphs [1]-[177].
[179] A method of producing a circular polyribonucleotide from the linear polyribonucleotide of any one of paragraphs [1]-[174], the method comprising providing the linear polyribonucleotide under conditions suitable for self-splicing of the linear polyribonucleotide to produce a circular polyribonucleotide.

1. A circular polyribonucleotide comprising, in the following order from 5′ to 3′:
(a) a first post-circularization element;
(b) a first spacer element having a length of at least 100 ribonucleotides;
(c) a polyribonucleotide cargo;
(d) a second spacer element; and
(e) a second post-circularization element;
and wherein the first post-circularization element and the second post-circularization element together form a circularization junction.

2. A circular polyribonucleotide comprising, in the following order from 5′ to 3′:
(a) a first post-circularization element;
(b) a first spacer element;
(c) a polyribonucleotide cargo;
(d) a second spacer element having a length of at least 100 ribonucleotides; and
(e) a second post-circularization element;
and wherein the first post-circularization element and the second post-circularization element together form a circularization junction.

3. The circular polyribonucleotide of claim 1 or 2, wherein the first spacer element has a length of between 100 to 500 ribonucleotides.

4. The circular polyribonucleotide of claim 3, wherein the second spacer element has a length of between 100 to 500 ribonucleotides.

5. The circular polyribonucleotide of claim 4, wherein the first spacer element and the second spacer element each comprise 110 to 500 ribonucleotides.

6. The circular polyribonucleotide of claim 5, wherein the first spacer element and the second spacer element each comprise 120 to 500 ribonucleotides.

7. The circular polyribonucleotide of claim 6, wherein the first spacer element and the second spacer element each have a length of 150 to 500 ribonucleotides.

8. The circular polyribonucleotide of claim 7, wherein the first spacer element and the second spacer element each have a length of 200 to 500 ribonucleotides.

9. The circular polyribonucleotide of claim 4, wherein the first spacer element and the second spacer element each have a length of 100 to 300 ribonucleotides.

10. The circular polyribonucleotide of claim 9, wherein the first spacer element and the second spacer element each have a length of 100 to 200 ribonucleotides.

11. The circular polyribonucleotide of claim 9, wherein the first spacer element and the second spacer element each have a length of 110 to 300 ribonucleotides.

12. The circular polyribonucleotide of claim 11, wherein the first spacer element and the second spacer element each have a length of 120 to 300 ribonucleotides.

13. The circular polyribonucleotide of claim 12, wherein the first spacer element and the second spacer element each have a length of 150 to 300 ribonucleotides.

14. The circular polyribonucleotide of any one of claims 1-13, wherein the difference between the length of the first spacer element and the length of the second spacer element is 0 to 500 nucleotides.

15. The circular polyribonucleotide claim 14, wherein the difference between the length of the first spacer element and the length of the second spacer element is 0 to 100 nucleotides

16. The circular polyribonucleotide of claim 15, wherein the difference between the length of the first spacer element and the length of the second spacer element is 0 to 50 nucleotides.

17. The circular polyribonucleotide of any one of claims 1-16, wherein the first spacer element or the second spacer element consists of:
(i) a polyA region comprising 80% to 100% adenosine residues;
(ii) a polyAC region comprising between 80% to 100% adenosine or cytosine residues;
(iii) a polyAU region comprising between 80% to 100% adenosine or uridine residues; or
(iii) a polyAG region comprising between 80% to 100% adenosine or guanosine residues.

18. The circular polyribonucleotide of any one of claims 1-17, wherein the circular polyribonucleotide exhibits at least 2-fold greater stability in comparison to a polyribonucleotide without the first spacer element or the second spacer element.

19. The circular polyribonucleotide of any one of claims 1-18, wherein the circular polyribonucleotide may be detected for at least 2-fold longer in comparison to a polyribonucleotide without the first spacer element or the second spacer element after the circular polyribonucleotide is administered to a subject.

20. The circular polyribonucleotide of any one of claims 1-19, wherein the polynucleotide cargo encodes a polypeptide, wherein the polypeptide has at least 4-fold greater expression in comparison to a polyribonucleotide without the first spacer element or the second spacer element.

21. The circular polyribonucleotide any one of claims 1-20, wherein the polyribonucleotide cargo comprises an expression sequence.

22. The circular polyribonucleotide of claim 21, wherein the polyribonucleotide cargo comprises an IRES operably linked an expression sequence.

23. The circular polyribonucleotide of claim 21 or 22, wherein the expression sequence further comprises a 3′ untranslated region or a 5′ untranslated region.

24. The circular polyribonucleotide of any one of claims 21-23, wherein the expression sequence encodes a polypeptide.

25. The circular polyribonucleotide of any one of claims 1-24, wherein the polyribonucleotide comprises between 500 and 20,000 ribonucleotides.

26. The circular polyribonucleotide of claim 25, wherein the polyribonucleotide comprises between 2,000 and 20,000 ribonucleotides.

27. The circular polyribonucleotide of any one of claims 1-26, wherein the circularization junction comprises a splice junction.

28. The circular polyribonucleotide of claim 27, wherein the first post-circularization element comprises a first exon fragment and the second post-circularization element comprises a second exon fragment, and wherein the first exon fragment and the second exon fragment are joined by the splice junction.

29. The circular polyribonucleotide of any one of claims 1-28, wherein the circularization junction comprises an oligonucleotide splint that is hybridized to the first post-circularization element and to the second post-circularization element.

30. The circular polyribonucleotide of claim 29, wherein the oligonucleotide splint is a DNA splint or an RNA splint.

31. The circular polyribonucleotide of claim 29 or 30, wherein the first post-circularization element comprises a region that is capable of hybridizing to a first region of the oligonucleotide splint and the second post-circularization element comprises a region that is capable of hybridizing to a second region of the oligonucleotide splint.

32. A linear polyribonucleotide comprising, in the following order from 5′ to 3′:
(a) a first post-circularization element;
(b) a first spacer element having a length of at least 100 ribonucleotides;
(c) a polyribonucleotide cargo;
(d) a second spacer element; and
(e) a second post-circularization element;
and wherein the first post-circularization element and the second post-circularization element together form a circularization junction.

33. A linear polyribonucleotide comprising, in the following order from 5′ to 3′:
(a) a first post-circularization element;
(b) a first spacer element;
(c) a polyribonucleotide cargo;
(d) a second spacer element having a length of at least 100 ribonucleotides; and
(e) a second post-circularization element;
and wherein the first post-circularization element and the second post-circularization element together form a circularization junction.

34. The linear polyribonucleotide of claim 32 or 33, wherein the first spacer element has a length of between 100 to 500 ribonucleotides.

35. The linear polyribonucleotide of claim 34, wherein the second spacer element has a length of between 100 to 500 ribonucleotides.

36. The linear polyribonucleotide of claim 35, wherein the first spacer element and the second spacer element each comprise 110 to 500 ribonucleotides.

37. The linear polyribonucleotide of claim 36, wherein the first spacer element and the second spacer element each comprise 120 to 500 ribonucleotides.

38. The linear polyribonucleotide of claim 37, wherein the first spacer element and the second spacer element each have a length of 150 to 500 ribonucleotides.

39. The linear polyribonucleotide of claim 38, wherein the first spacer element and the second spacer element each have a length of 200 to 500 ribonucleotides.

40. The linear polyribonucleotide of claim 35, wherein the first spacer element and the second spacer element each have a length of 100 to 300 ribonucleotides.

41. The linear polyribonucleotide of claim 40, wherein the first spacer element and the second spacer element each have a length of 100 to 200 ribonucleotides.

42. The linear polyribonucleotide of claim 40, wherein the first spacer element and the second spacer element each have a length of 110 to 300 ribonucleotides.

43. The linear polyribonucleotide of claim 42, wherein the first spacer element and the second spacer element each have a length of 120 to 300 ribonucleotides.

44. The linear polyribonucleotide of claim 43, wherein the first spacer element and the second spacer element each have a length of 150 to 300 ribonucleotides.

45. The linear polyribonucleotide of any one of claims 32-44, wherein the difference between the length of the first spacer element and the length of the second spacer element is 0 to 500 nucleotides.

46. The linear polyribonucleotide of claim 45, wherein the difference between the length of the first spacer element and the length of the second spacer element is 0 to 100 nucleotides.

47. The linear polyribonucleotide of claim 46, wherein the difference between the length of the first spacer element and the length of the second spacer element is 0 to 50 nucleotides.

48. The linear polyribonucleotide of any one of claims 32-47, wherein the first spacer or the second spacer element consists of:
(i) a polyA region comprising 80% to 100% adenosine residues;
(ii) a polyAC region comprising between 80% to 100% adenosine or cytosine residues;
(iii) a polyAU region comprising between 80% to 100% adenosine or uridine residues; or
(iv) a polyAG region comprising between 80% to 100% adenosine or guanosine residues.

49. The linear polyribonucleotide of any one of claims 32-48, wherein the polyribonucleotide exhibits at least 2-fold greater stability in comparison to a polyribonucleotide without the first spacer element or the second spacer element.

50. The linear polyribonucleotide of any one of claims 32-49, wherein the polyribonucleotide may be detected for at least 2-fold longer in comparison to a polyribonucleotide without the first spacer element or the second spacer element after the polyribonucleotide is administered to a subject.

51. The linear polyribonucleotide of any one of claims 32-50, wherein the polynucleotide cargo encodes a polypeptide, wherein the polypeptide has at least 4-fold greater expression in comparison to a polyribonucleotide without the first spacer element or the second spacer element.

52. The linear polyribonucleotide any one of claims 32-51, wherein the polyribonucleotide cargo comprises an expression sequence.

53. The linear polyribonucleotide of claim 52, wherein the polyribonucleotide cargo comprises an IRES operably linked an expression sequence.

54. The linear polyribonucleotide of claim 52 or 53, wherein the expression sequence further comprises a 3′ untranslated region or a 5′ untranslated region.

55. The linear polyribonucleotide of any one of claims 52-54, wherein the expression sequence encodes a polypeptide.

56. The linear polyribonucleotide of any one of claims 32-55, wherein the polyribonucleotide comprises between 500 and 20,000 ribonucleotides.

57. The linear polyribonucleotide of claim 56, wherein the polyribonucleotide comprises between 2,000 and 20,000 ribonucleotides.

58. The linear polyribonucleotide of any one of claims 32-57, wherein the circularization junction comprises a splice junction.

59. The linear polyribonucleotide of claim 58, wherein the first post-circularization element comprises a first exon fragment and the second post-circularization element comprises a second exon fragment, and wherein the first exon fragment and the second exon fragment are joined by the splice junction.

60. The linear polyribonucleotide of any one of claims 32-59, wherein the circularization junction comprises an oligonucleotide splint that is hybridized to the first post-circularization element and to the second post-circularization element.

61. The linear polyribonucleotide of claim 60, wherein the oligonucleotide splint is a DNA splint or an RNA splint.

62. The linear polyribonucleotide of claim 60 or 61, wherein the first post-circularization element comprises a region that is capable of hybridizing to a first region of the oligonucleotide splint and the second post-circularization element comprises a region that is capable of hybridizing to a second region of the oligonucleotide splint.

63. The linear polyribonucleotide of any one of claims 32-62, wherein:
the first circularization element comprises a first catalytic intron fragment, a first splice site dinucleotide, and a first exon fragment; and
the second circularization element comprises a second catalytic intron fragment, a second splice site dinucleotide, and a second exon fragment.

64. The linear polyribonucleotide of claim 63, wherein the first catalytic intron fragment and the second catalytic intron fragment, together, are capable of self-splicing thereby covalently joining the first exon region and the second exon region to produce a circular polyribonucleotide.

65. The linear polyribonucleotide of claim 63 or 64, wherein the first catalytic intron fragment and the second catalytic intron fragment are from a cyanobacterium Anabaena pre-tRNA-Leu gene or a Tetrahymena pre-rRNA.

66. The linear polyribonucleotide of any one of claims 32-65, wherein the first circularization element comprises a region that is capable of hybridizing to a first region of an oligonucleotide splint and the second circularization element comprises a region that is capable of hybridizing to a second region of the oligonucleotide splint.

67. A DNA vector comprising an RNA polymerase promoter operably linked to a sequence that encodes the linear polyribonucleotide of any one of claims 32-66.

68. A circular polyribonucleotide produced from the linear polyribonucleotide of any one of claims 32-66 or from the DNA vector of claim 67.

69. A pharmaceutical composition comprising the linear polyribonucleotide, the circular polyribonucleotide, or the DNA vector of any one of claims 1-66 and a pharmaceutically acceptable excipient.

70. A method of expressing a polypeptide in a cell or a subject, the method comprising providing to the cell or the subject the linear polyribonucleotide, the circular polyribonucleotide, the DNA vector, or the pharmaceutical composition of one of claims 1-69.

71. A method of producing a circular polyribonucleotide from the linear polyribonucleotide of any one of claims 32-66, the method comprising providing the linear polyribonucleotide under conditions suitable for self-splicing of the linear polyribonucleotide to produce a circular polyribonucleotide.


SEQUENCE LISTING (338 sequences)

SEQ ID NO: 1 - RNA, 201 nt, Barley yellow dwarf virus
  source 1..201: mol_type = genomic RNA
gtcttagcaagctctgagccaggagatggacataaaccatagcaatccaacgtgtaaccgcaatggggcaaacaacaggtgaaccgtgtccacgggcctggttaccgaaaggaaagccagtatccaacacagcaatgtgttgggggtcacaccttcggggtactcttaacgctgacactcgaaagagcagttcggcaaccc

SEQ ID NO: 2 - RNA, 305 nt, Tobacco necrosis virus D
  source 1..305: mol_type = genomic RNA
tcgctttcatagatccgtcttcccagagacgttaagaagaagctggagaaaaatattaggttagaagcttgggcgtgacaaacccaagttgcatctcttacgtggttaatcacactgtatgttgacgaataggccggatcctgggaaacaggtttaacgggctctctgtggtggagggccgacgcatcacctatttgtgctccaacagtggttgtcatcacgtgtcctgacatggctccatgcgacagcatgggggggtccagagtcagtcccctctttatttacctaggatttcctaggaaccc

SEQ ID NO: 3 - RNA, 230 nt, Olive latent virus 1
  source 1..230: mol_type = genomic RNA
cgtaactgtgctaatccctaaaagttttagtagagcggtaagcaatccggatcctggtagacaggtttaacgggctgggggtggtgccccggccgtcgcatcacttgctgatacaatctacaagactcatgggagggtctgcacaggtatccacgccgaggacgggacgaaagtctcggacggggggtagtggacccaacaccagattgaggggcttttgccccacccca

SEQ ID NO: 4 - RNA, 210 nt, Leek white stripe virus
  source 1..210: mol_type = genomic RNA
tcacctcacggttttgttaattacattcaagtcgaagttgcatagatcccgagaacgggttttaacgggctcctgtggtgggagccgacgctcatcgcttctgaaatttgaatggttgtcacaaaacaacatatgatggctcactacaacagtagtggggggtccaggcatatgtcccgcaatacttatagctaccttttggtagtaccc

SEQ ID NO: 5 - RNA, 376 nt, Sweet clover necrotic mosaic virus
  source 1..376: mol_type = genomic RNA
tgcaagcgtaacctccatccgagttgcaagagagggaaacgcagtctcgccgaccctgttgccaaacagtaaaattggaaaaaatagagtgttaggagtagttcccgtacccgcgggacaagaacctattgtagtagacgaaccggtctcggaccctggtaaacaggtacctagcgtaatagaaggtcgctagaaatcgggtgggcttcacctacgggtaacccaccgtcgagagggaaaacctctgtgtggttgtgcgcacgtcgtttcctgaagaatctaccttcggttaaacaaaattggcccctgagagtagaaaactcaggttaataaaacaggagattatttgcttagtcgtataacggctaagcacccc

SEQ ID NO: 6 - RNA, 645 nt, Tobacco bushy top virus
  source 1..645: mol_type = genomic RNA
attggacacaaaacggagctagatttagtgttgcaattgctagaccgttagccactacgccagacagtgcgataactgaggtcgtctatgacccacagggtgccatcctgtgcaatttccaacccttggcaggggtgatcctagaggacgcgggcattgacttgcctggggatgcttctagttacataccggagtggttgatagacctctgtgtaaggaaacaggtgtgagactggatcctgggaaacaggctgggatggtcccgtaagttcgggcggtgcccttcgggggtaaccccaccgccgacgtatcacctcacacgctgttggccgataagccgaggtgttggaaccgcaccactctggaggttccctctccttatgaggtggaggcacagcacgccttaagtgtctgcgatccggcgttcacgtgccggtggtccccctagttaacgtggtgccacagtctgtggtaggggcagctggtcggattccagcagaaaccaagtagcaaatggacgttgctacctcgagcctagagtcatgggcccgcatcgacgtttgcggtaactcacagcgggcgataatagctgttgagaatgtcggttcctgacttagcgggagatgagcactttctctcgcgccc

SEQ ID NO: 7 - RNA, 645 nt, Tobacco bushy top virus; identical to SEQ ID NO: 6
  source 1..645: mol_type = genomic RNA

SEQ ID NO: 8 - RNA, 535 nt, Groundnut rosette virus
  source 1..535: mol_type = genomic RNA
aactacgaccccgccaggggttaattctaccacaggttggcagctgtgggatcccagtccaggtgtctttaactagatgtggatggactggttgattttgtcggccttgataggggacgaaaccacgagtcaagcggaagagatagggggtgttggatcccgggaaactggcagctgggcagcgtaagcacgggtgtgtgaaccccacaccgacgtatcaccccccttggacttagtacgcgatttgaggcggttgttccacgtgggaagcaggctcggcacccaatccgacagggaaaagtcgtaggctccggcgctgcgggagggtaaactagcggccgatacgcattgtgcagtgcgttggtcccactcagcacatggcgctctgccatagggacggaagggccgtggtactcccttcaacaggcggcacccagtgaggctcgccaatccatatgactaggattatgctgagtgataccccctttcgaacgtacggacgtccagctgagatctcctagataacatgacaccc

SEQ ID NO: 9 - RNA, 238 nt, Olive mild mosaic virus
  source 1..238: mol_type = genomic RNA
gtgttagttaactggcttaaaccctaagggtagatatgtgggcggtaagcaagagggatcctgggaaacaggcttcgacgggttgggggtggtgccccggccgacgcatcacttgctgatacaaccattagacacctaagggcgggtctagccaggtctccacgccatgatcaattggaaacgattgtgaggggggtagtggaacccataccagattgaggggcctttgccccacccc

SEQ ID NO: 10 - RNA, 299 nt, Beet black scorch virus
  source 1..299: mol_type = genomic RNA
atcccacatcctggtgtggttaatccagtgaaaaatattaggaaatccttgggattcacaccccagggaggattgcttgcatagtgcaggtatgttgagttacactatgaggtattggtgctgaatcctggtaaacaggcttgacaggtttgggtggttccagaccgatgtattacccaagatactcatggtactctactagaaaacactatgcgtgcgcacacggctggctatgtcctatcatagctgggggccccggagtgcgaaaccctcttatatacctggtcttccaggtgccc

SEQ ID NO: 11 - RNA, 167 nt, Rose spring dwarf-associated virus
  source 1..167: mol_type = genomic RNA
ggagccaattctaggcttcgcagtgctggatcctggtaaacaggcgttcaagtaacctaagcttcggtgaagggcaatgcttgttgtaagctcgggaggtcttgtcaacctccgtcgtatcacctgcgtacctcccctggataataacccatttctgcaccagacta

SEQ ID NO: 12 - RNA, 192 nt, Opium poppy mosaic virus
  source 1..192: mol_type = genomic RNA
taagctgactgacattcggttgttgcgacgcttagttataacatgggtgggtttagagttgcaatatagtgggagccgggtcctggtaaacaggttgagttggtctcgtaatctcgggtggactgccaatccaccgtcgtaccactctcatgggctattgcatcctctaatagggagtttcaggaacaccca

SEQ ID NO: 13 - RNA, 653 nt, Soybean dwarf virus
  source 1..653: mol_type = genomic RNA
tataggtagaagtgtgtttgcgctttccatcacacgaaattggagccaaccatggtgagttgctgttgtgggatcctgggaaacaggttcggtgaacaaaaagccggtaatctcgggcgtttgtcagacgccgccgaatcaacacagtatgcatatccatcaataacccaagttacgtgtgactaaaaattagaattagtttaccgcctctcttgtaaaccacatacaagacgtgagattgtggagagtattatcccttgaataaggataattgaaagtttctggtttcaatccattgatcaaaattgacactgcttctggtgattacactgccgctggtgatacagcgtaaacaatttctgcttttcgctctatgaaatgagagtgattagtaaagtcttgccgttcacatgtttatttgacggtaccaggcttcgcccctttgatggcgttaaatgaaagcggagtaaaccgacaccaccttaccaggtcgggttgacctggggaaaccgggaaaatacccgggacacttggtttagtggagtttaaactcgagggctcttcctcgttaaaaagggggtagccaggttgaaaactggcactagggagaaatccctctttgttgttaaggtggctttgtgtccacctgcccc

SEQ ID NO: 14 - RNA, 443 nt, Red clover necrotic mosaic virus
  source 1..443: mol_type = genomic RNA
ttggttcttttaagtgtagcctccacccgagttgcaagaggggaacacgcagtctcgccgaccctgttggcaaacagtaaaattgcaaaaaatagagtgctaggagtagttcccgtacccgcgggagcaagaccctactacagtagacgaaccggcatcggaccctgggaaacaggtacctagcgtattaataggtcgctagaaatcgggcgggcttcaccttcgggtaacccgccgtcgagagggaaaacctctgtgtggttgtgcgcacgtcgttgtcggaagactctcactgttgtagttatttcctttttcttttaggtaggagcacttgctcagagggcgcatacctctggtaaaacaaaattggcccctgagagtagaaaactcaggttaataaaacaggagaatatttacctagtcgtataacggctaggtacccc

SEQ ID NO: 15 - RNA, 91 nt, Bean leafroll virus
  source 1..91: mol_type = genomic RNA
ccactagtgcatcgaatcctgggaaacaggcacggagaacaaaatccggtaagctcgggaagccgtcaacttccgccgtattgatgaaaac

SEQ ID NO: 16 - RNA, 92 nt, Tobacco necrosis virus
  source 1..92: mol_type = genomic RNA
acaccattggagacggagacggatcctgggaaacaggcttgacgggcggggggtggtgcccccgacgacgcatcactccggataccaatggt

SEQ ID NO: 17 - RNA, 636 nt, Ethiopian tobacco bushy top virus
  source 1..636: mol_type = genomic RNA
actaacactgcccgggatggcaataaactactgtgcgtccgcaatgacgttaaactacggcagtctgcaatgactataaacctacggtcctgccaaaggactactcttttggggttggcatcccaagatcccacacccggtgtctttagctagacgtgggtggtgtggttacaatttgacagcctcggagtggcgatacagagagactaactgataaagtgggctagggcggatcctgggaaacaggctgcctggcggcgtaagctcgggtgtgtgaaccccacaccgacgcatcacctagccgggacttgacacggtagtttcacttattctcacatggacagggtctgtcacgttcccgactacgtaaagtcgtagactccagcgctgcgggagtataatttcgcggccgacgtgcattgctcggtgcactggtctcccctagagcctaacggtctgttagtgggatatgacccatggtgggtcgtaccgggtggtaaccggaataacaaccaaccaagactggccatcctaggctggagtaccatgcgactcccaaacctacttcgggcgtataatagaagtggtaatgcatggattagctaatagcggcgggaattcaccctcgtcgcgccc

SEQ ID NO: 18 - RNA, 167 nt, Rose spring dwarf-associated virus; identical to SEQ ID NO: 11
  source 1..167: mol_type = genomic RNA

SEQ ID NO: 19 - RNA, 420 nt, Ixeridium yellow mottle virus 2
  source 1..420: mol_type = genomic RNA
ccaggagcagcaaaacccagccccggagaacttgcaagagggggggggtaaagtgtttgtccgcgtgagtgcccttacgtgtgtctcggggcctagccagcccactctaactccacccttaggcaaggtggcgcactgtcggcgtgcgtatttcccgctgggttgacaaccagcagaccctacctggtcggggcttaactgactttcccaggtagttactttagtgtggtggagattgctgggtataggtgctggatcctgggaaacaggtttaacgggcggactagtcacccgccgtcgcatcacccttgataccagctcctcctcaccgtggggtggggaacacacttgcagtatatagttatgccaaaaatattcagccaacccaggttggcccactgtattgtccggtgcagggcc

SEQ ID NO: 20 - RNA, 652 nt, Patrina mild mottle virus
  source 1..652: mol_type = genomic RNA
caaccccaacaaactcagccgggaagaacttgcaagagccggagaggtagcgtttactccatgtctagctggcgcatggttttagtgtacatacaaatccagggatcagcgcctggcggcacaccacttgtcgcaactcagtggggatcccatggataggcacttagatgagtcgagtccatggttgagtttgggtgtcttgaaggatcggttggtcttgagattgaggcgtgagtgtgctggatcctgggaaacaggtttaacgggcgactagtcaccgccgacgcatcaccacaatatagcctctcaagtgtgggcacgggacccacagccaacagagggaagcagacatcctgtttacaaaaaccaaaaatagcttggtgacccaggtcaccccccctccattgtcgggtggttggggccttctcacgaccatgtgcaatattcagcacatgtgggtgtggtacacggtatggccacaagggagaaaaccctccaaatccgttgaccgtcgtcaggtcaccgacctgctggtcttagcaggcgttggatacgtacatccctggagcagcaccagaaagtcccgggcattacaatagggataaagctgtgagatcgtattttagcgtcgagtgcagcactcggcgcgccc

SEQ ID NO: 21 - RNA, 717 nt, Paederia scandens chlorosis yellow virus
  source 1..717: mol_type = genomic RNA
actgaaaccccaagccggaggaactgttgcaactgccggaatggaagtcgttcaagcccgtgaaggcttataaactaaatggccacagaaggcaactgtggacagaaattgtatagacacggagtaaataccgtggtgccgaccaactcggctgcatctcttgctggtgtggactaccagtgaccccgggggcggatccattaagttggcttgggatcccccggttaaataggaacggtagttggcttagacggaatggttggagtggaaaacctgatgctggatcgcctcctgggaaacaggcagcatggcgcataagatcgggggaggttggcccgcctctccgacgtagggtaccagcattgagaaggagtacaaccaccttagtacgtcgaagcttggataagactgtttccaccctacccacagggttacagtggtagaggactgtgggcttttcccacagcgattggattagtgtgtccatggtctcccaagtcaacactttcgccttgtgcgtatgggagtgggttggtttccccacacgtctccacagacgttaaacctggccgtccaccaagacgttaaactaggaaccggttggtgaccgtcaacacaccctggcagaggacgccagagcgactcccgggcattataatagggactcgccaagttctcatctgttcttagcggttgttatcactgcaaccgcgcccg

SEQ ID NO: 22 - RNA, 151 nt, Picris umbravirus 1
  source 1..151: mol_type = genomic RNA
ggctccccatccttcggggcttgaaggacgttggacgcgataagtctccagagtggatctggcagcccccagggctataatatgggggaatccaccagtttgaacctagcgagcagcctaagaaggtacttgttcgatctggtaatgttat

SEQ ID NO: 23 - RNA, 622 nt, Satellite tobacco necrosis virus
  source 1..622: mol_type = genomic RNA
taatcccagaggttcacaatgttagtgatgggcgctgaaagatgcgtagctacccttctggagccacttcctggtggtaagcagaaatccaagggtacggtggtacggtggaaagcagtcccagctctgcattgggaaccggctcacacccagcttagggctaaagtgtactacttgctcatttgtagtctaaatgagacgttggcctcgccgtgtcgaggtggcctaagggattggaacccctgatggtcgtagtcgaatttcccgtgtttcatcccgagtctcttggtcataatgccattagtaggtcaagcacccaacgtaacttcaaagatatcctccttgcaacaagaatatgtgcgctgtctgtgtttaaagcggtatattttgggcgccggcatatcgttgcttggaccagggccccacgccggttggtacccgggtggcttcccctcgttcacagggctttaggagatgataaggtatagttactaggcaaatgcggacaaacctgaaaagctcgctagtggtgggctggccaagcgaagaacctcatccaggtatagttctacatgggaaatttggtaccatccaaacttctatgaagtcctcgactaacccc

SEQ ID NO: 24 - RNA, 268 nt, Pelargonium line pattern virus
  source 1..268: mol_type = genomic RNA
agcttacatcaacaagctgtgaggaaagccactgcggtggggtcagtctcctggtgcgccaacacccagaaggggagacaacttcagtggttcaaaacccgatgcttcgtgtaagcctagaaaaatccaaaaacagagtgctactgctttcagtgtgcgagctttatttgctcacttcctccgtcgataaaatcccgaattttcgggtgggcttcggcggacatccttaatgaagaacgggggggtaacccacccccctgatctgccc

SEQ ID NO: 25 - RNA, 100 nt, Pelargonium chlorotic ring pattern virus
  source 1..100: mol_type = genomic RNA
gcacatgtagttcccctaacctctcagtaggagtcaagccctgatgcgccaacatccagaagggggtaaagctgagtcggtgcaaggaaccttgttagcc

SEQ ID NO: 26 - RNA, 112 nt, Pelargonium chlorotic ring pattern virus
  source 1..112: mol_type = genomic RNA
ataattataatcatgcaatggcacatgtagttcccctaacctctcagtaggagtcaagccctgatgcgccaacatccagaagggggtaaagctgagtcggtgcaaggaacct

SEQ ID NO: 27 - RNA, 99 nt, synthetic construct
  source 1..99: mol_type = other RNA
ccacgcgtatgtactagaatgtgagttgctggagtcagaccctggtgcgccaacacccagaagggggtcaaccaacttgcttttagttactgcgtgcgg

SEQ ID NO: 28 - RNA, 151 nt, Pelargonium ringspot virus
  source 1..151: mol_type = genomic RNA
cttatgtttgccgtatgtgaatggtgggatgggggggttttggtaatcattagggctacaggacaaccgaggaggcgccaaccttcgaccatcggtgatcccctattgcactccagccacccattgttaccttcactaggcaccacttaag

SEQ ID NO: 29 - RNA, 90 nt, Calibrachoa mottle virus
  source 1..90: mol_type = genomic RNA
ctaagagttactgccaccaaaagaggataatcagaccacttgtagtttgctgccacaaactggttgacaagttaatgcctctctcttagt

SEQ ID NO: 30 - RNA, 122 nt, Saguaro cactus virus
  source 1..122: mol_type = genomic RNA
gcttgacgtggtttgttgctcgactgtgaggggacctacccactgtgctgccacacaggaacttccaaccttcgggtggcgaggtagggcagaagagtgactgaacgaggatggtgtagagc

SEQ ID NO: 31 - RNA, 236 nt, Pelargonium flower break virus
  source 1..236: mol_type = genomic RNA
gagggcagtcaccctaccttctgccagaggttcccggaggagtagtgatcctttgggggtgattcatagctaggaaaggtcggagagttgggaggaggccaactgggggaggtgctaggagcaccagatgcagcagagcaaaaatccgggtttcatcgcctggtgcgggcgctcaatgctgctaacacgttcctgaaggaagaaaggtttcggtcgatggagaccttaacccgccc

SEQ ID NO: 32 - RNA, 130 nt, Carnation mottle virus
  source 1..130: mol_type = genomic RNA
atgcaatagaaaagtttgttagacacacacggcgggacgtgttgccggagctgccactccgcccaagatggatcgatattaaccatctttggcaacagcgtgcgtggctaatcgaacctagtaattgcat

SEQ ID NO: 33 - RNA, 148 nt, Honeysuckle ringspot virus
  source 1..148: mol_type = genomic RNA
ccaaccacgactgccgttaaagagcttcaatggtatgtctcaagactatagacgggatgagcttctgggggctgccaccctcgcctcatgcgcattgcctgctctgggcgcatgtggaagctgatatagttgagagcttcgtggaagg

SEQ ID NO: 34 - RNA, 140 nt, Pea stem necrosis virus
  source 1..140: mol_type = genomic RNA
taacacagcaatggagtaagtgtactctggtagcaatgggtggggtcgatttcctacgtctagctttggccggctagcgtctctcgtagtggggaaataactcattgagaccagctcagaacgctgacattgtctgtgtt

SEQ ID NO: 35 - RNA, 240 nt, Hibiscus chlorotic ringspot virus
  source 1..240: mol_type = genomic RNA
gtgtgtattatatgggcctccgtgctgttaggggcagtggaaacgtcggtctagccaaccgtcccctgggtagtgtgctccgtctaagtacaccactactcggtttccacaaacgatcggactcccaaaatagtgtacattatgttgattgcttctttcctcctttgttatatctcctctcccctctcctctacacttggaatccataaaatccttcgggtgggcttggatttcgggtac

SEQ ID NO: 36 - RNA, 163 nt, Galinsoga mosaic virus
  source 1..163: mol_type = genomic RNA
gtgatcttctctagaggaagatgcttgggggtgttcccctaaagttcagaatggtctatcagggagcaggcacttttccggctttggcgagtcgtctccacttgtatacgaacagtgaggggaaaagttatctgatagaagaatgaaccatggacaaggacac

SEQ ID NO: 37 - RNA, 194 nt, Cocksfoot mild mosaic virus
  source 1..194: mol_type = genomic RNA
acctgttaaacagactcacctcgggcacgcgctgaaagtgcccgttggaaggattgcgatgggccagtattcctactgtaccagagaacgtgtcatcaggggatgaccatgtccaccttgccgggtgccctcggacctatgtccgtgacatggcgtgacgctagtaatggtgctatggaagaaacacctaaccc

SEQ ID NO: 38 - RNA, 416 nt, Thin paspalum asymptomatic virus
  source 1..416: mol_type = genomic RNA
caggcagtcgggttgcaactcggcctcctacgcatgagctatgtcattgagctcaggggagccactgcaggcagtggtgccagcgcatagactcgctcagagtcatcctaggctaggaccggccagacaaaggtccgacactacatgcagcgctctaggtgtgtagtgacagtaagcgcgctctatccgaaactccagtgacaggacaagaccccaggcgggacgccacacccatgtttgcagacatggcccgaggaaactctggtgtgggctggggggaatcctatgatcactggacggaaggtagagagttccagcacaacccctttagggtgggccgtaagtgcgtgggaacatttcctacagtctcactgagaagattggacggggtaccatgagtacccctcaccaggccc

SEQ ID NO: 39 - RNA, 271 nt, Japanese iris necrotic ring virus
  source 1..271: mol_type = genomic RNA
aaacgaccattcttgggtttgcagcgagatgccacgtgccagaggatagtaccactggctgaccctaccagcgtttgcgagttggtatgcgtggatgtaaattccaagtcttgtgtccgacctaggctccgccggctggagctcgcatgtggcagtgcgagcagaggatgtcccgatgcataaaatcccgtgagggtgggcttgctagggaggccgacactgcgtaatgcaaaactacccttttcccctgtgagggggaagaggggggccc

SEQ ID NO: 40 - RNA, 206 nt, Pea enation mosaic virus-2
  source 1..206: mol_type = genomic RNA
gtgagctcccgcagagggataagctgtctccctgcgacgtagtagtagaacacgtgggataggggatgaccttgtcgaccgtttgtcggtcccctgctccttagagctggcaaggcgcccattggttctacatttctaccaaagttggtggtggatgtctcgcccaaaaagatcataaacgcgcgggataaggccctctccacctt

SEQ ID NO: 41 - RNA, 646 nt, Panicum mosaic virus
  source 1..646: mol_type = genomic RNA
ggttctatccgaactacagcacggaaacacctaagaccctcacagacctgatggacagcgaatccctcgtgttagtcccttccctctccgggaaaacttaccgccctaagatcgaaacacgaggaaacccaccggaattgcgcaacatagatgccaccgctttctccgctctcagcgacgaagataagggtgactactcggtgggaaggctggttgttggctccagcaaacaagctgtcgtgatacagctcggactcctccgcatgagatactcagctgaaatgcggggagcaacctccatcagtggggttagcgcataaacctgcccaaggttatcacaggctaggaccggcctgataaaggtccgacttgatgtgcagcttcttaggtgcatctcgtatagaagcgctctggacatgacacctttgagacagaccgtacagcagtcacacgggacgccacaccacctttgcagaggtgcccttgggaaaccaatggtgtggggtgacactgattagtcgttaacggtgtactaatccagagtagttcagtgcaggggaaacccgggccgtaagcacgtggaactatataacttaatctcgctgagaagattgggggggattcatgaaatcccctcaccaggccc

SEQ ID NO: 42 - RNA, 337 nt, Maize chlorotic mottle virus
  source 1..337: mol_type = genomic RNA
ggtgagccggcatgaggttgcaagaccggaacaaccagtccttctggcagagtcctgccaatccaaagtgtttgggagcctaaatcgtatactagtagtttgcgtgatgaccatgactggagagtgggcggcggctgccaaccgcagactgggcgtatatagtaagccttgacccacccatggacaacacgtacttacgaacggtgcgacatggtaactggatacacttaaccctggggcaagtagatgctaggaaactagcatcgggccgcccacgagggtttctgaactcaacggagtagaacggggggggggtaatgcccctctcttccggccc

SEQ ID NO: 43 - RNA, 311 nt, Bermuda grass latent virus
  source 1..311: mol_type = genomic RNA
gctaggatggctatcctatcttattggtgacagagtcaccctgggaagcacacccctttagtgctcccagtatgggggcttgtcggaccacccctccattagcgctgtggtgtcttgtctcttgcctttgccgaggcacaaatgctgcatagtagcagtgggagacaacagtacgcgaccggggatatagagtagactgacaagtagtctggggaaaaacacaagggaaaccttgtggggcacccgcgagactatagtatctcacctgagatgataggagttccctcacgaggggaacccacctcctgccc

SEQ ID NO: 44 - RNA, 172 nt, Adonis mosaic virus
  source 1..172: mol_type = genomic RNA
aagtatagaacgacacgttaaacagctgagcacatgccagggtggtctggcgtccccaacccataaagatcctagaggaaactctgggtgagggcttggtagtagggacaaccaccgtgtgattaaaatcacaaaacactagcggttagagttcatagctcttctcgcgccc

SEQ ID NO: 45 - RNA, 227 nt, Cucumber leaf spot virus
  source 1..227: mol_type = genomic RNA
tggcctagatcggaggggtcgtggtaatagcaccgttggtggttcttcggatgcttctcagtcccaaggcgcacaagcatgggggggttaccataacgatctaggagacagcttatccgagtactctgtgtctgacttgtagagagaccaccaatgcacaccagaaatgggggctataagtgtattggttcttaattttcatcccttgcgtggaaacacgctagccc

SEQ ID NO: 46 - RNA, 301 nt, Pothos latent virus
  source 1..301: mol_type = genomic RNA
gatgtcgctggaatcatccaagggcggtggcatgaagtcacatcagaagaaaaatagtaagcctaatggccatagtaggaggggtaatctatctggggaagttggtggttcttcttcctccttgccctcaggtgcacagactggggagtggatagataatgactatggagacagcttatctgagtattctggtgtgtcagcatagcacaccccaggtgtcataccctgaagtttagtaaggggggctataaatgacgcttggttttacctgaaatcccttccatgctttgcatggtagccc

SEQ ID NO: 47 - RNA, 221 nt, Maize necrotic streak virus
  source 1..221: mol_type = genomic RNA
gacaaactctccgcctaatgtctgatgatgtgaggaacgtggactgtgatgtggtggtgcggtaccatggctggtcaccatggtaatgcgtagggcaacacagttcattaagactcactgatgatggcactaggcacggttcacccccatccttcgggagggctatagggggtgaccgggttacaccaccggaagaccggaacattgcctttgggcagccc

SEQ ID NO: 48 - RNA, 323 nt, Melon necrotic spot virus
  source 1..323: mol_type = genomic RNA
atctcgccttaggtttacggttacccgagcttcgcctgaaacggcgttcctctaattccttcctggttctttccctccacggctcgcgtgttctgcggaactgcatctgcttcacttccttctttttccctctgtggtggtacgaccaatgggtggctcccattgactgcgtaggacatggaaaaagattgagtggagtgcacgaagcgtcgcaagatcgctgcctgacgaggtccagccaatcaaataccaaacatctggtttgggggcggattggctgtatgtcatgtaagtttgaggggttctatcggagccccccgccc

SEQ ID NO: 49 - RNA, 329 nt, Cucumber Bulgarian latent virus
  source 1..329: mol_type = genomic RNA
aaagcgaacttgacagactctcaagcaaggtatgaagttaaggagtggaacccggattaattgtaacctgtaattattctcgtggtggtacgacctcaggctggtcacctgggactgcgtaggacgggaggtgtcctcttatgggcgctagttattggtgaaacggggagacgggtggggacgacacggttgtatatagtccacaacacccacctgttgcaaactctggaagatagtatctcgcacggttacaccccatccttcgggagggctttaggggtgtgctggaagcaccaccggacagccggaacattgccgaaaggcagccc

SEQ ID NO: 50 - RNA, 225 nt, Maize white line mosaic virus
  source 1..225: mol_type = genomic RNA
cgtcctgtccggtggtaagggtcaagcgacagaaaagaggattttggctggtggtggtacggcccggggagtggttcccccgggatgcgtagcgccagctgaaggaatcccagtaatcgccactatagaagaccactaggacagcatgtactccacgcttcggcggggctataaggagtacatgatacccccccctatctttcacccagcttgctggggtagccc

SEQ ID NO: 51 - RNA, 202 nt, Johnsongrass chlorotic stripe mosaic virus
  source 1..202: mol_type = genomic RNA
cgtcgtcgctgtgttgccgaagcagaaagccctaggaaagggcctaaagcctgttggtggtacgactcctggtttggtcaccgggaactgcgtaggaacagactgaaggtcactagtactggcactatggcggcataagcgacacacggagccacacttcggtggggctataaggttccgtgttgtatccctcttactttca

SEQ ID NO: 52 - RNA, 158 nt, Gompholobium virus
  source 1..158: mol_type = genomic RNA
ctgtgccaaacttcctttgcaagatgcgactctctttgggcccacgtcaggactagccatcccaaggactagtggaaagagagaagttgttatgcagaggatatgcgtccactattgtgaagacgttaaaagcatacagtcgccatcgcaatggtagt

SEQ ID NO: 53 - RNA, 329 nt, Cucumber Bulgarian latent virus; identical to SEQ ID NO: 49
  source 1..329: mol_type = genomic RNA

SEQ ID NO: 54 - RNA, 370 nt, Tomato bushy stunt virus
  source 1..370: mol_type = genomic RNA
gacagactcttcagtctgagtttgtggagatgagtgtaaatctggcatagcatacaggttactcttgttgggttctggatgttaggatgacgagtcgactcgggctccgcactaggtttggtcgcctaggggatggagatatggaaagggtctcgtgtggtatcagtcggtcgaaagacgcgcttccaacatgggcctatggtcggataagtcttagcaataccagccagcatgaattggattcctgtttacgaaagttaggtgtcacttgtggaagcggacccagacacggttgatctcacccttcggggggctatagagatcgctggaagcactaccggacaaccggaacattgcagaaatgcagccc

SEQ ID NO: 55 - RNA, 369 nt, Carnation Italian ringspot virus
  source 1..369: mol_type = genomic RNA
gacagactcttcggtctgaggatagtggaagtgagtgtgcctagtatagcatgtaggttactctcctaggattatgtactgtgtgttaacgagtcacgtcgggctccgcgatgggtttggtcgcccatgggatggagatatggaaagggtctcgcgcactataagtctaactacggttgagcttgtgatgtgggcctatgcgtggataatacacacatgttgcaccctaggtgaattggattcttacttacgaatgccaaggacacttgtggaagctactgacgacacggttgatctcacccttcgggggggctatagagatcgctggaagcactaccggacaaccggaacattgcagaaatgcagccc

SEQ ID NO: 56 - RNA, 370 nt, Cymbidium ringspot virus
  source 1..370: mol_type = genomic RNA
gacagactcttcagtctaggttggaggaagcaggagaacactgcttgagggtacaggttactcttagtgagactaggttcgtgtgatgacgagtcaggtcgggctccgcgctgggtttggtcacccaggggatggagatatggaaaggatctcgtgcgctataagtctaacgaaagttgagcttgcaacatgggtctatgcctgaataagtcacgtgagttgccaccactatgaataggatttttgttcacgactagcggagaacatttgtggaaaccaaccaagacacggttgatctcacccttcgggggggctatagagatcgctggaagcactaccggacaaccggaacattgcagcaatgcagggg

SEQ ID NO: 57 - RNA, 348 nt, Cucumber necrosis virus
  source 1..348: mol_type = genomic RNA
gacagactcttcagtctgacttggtggaatcttgcgaatttaactgttactcttcatgggttccttcccatacgatgacgagtcaggtcgggccctatcttaggtttggtcacctaggggacggggatatggaaatcactttcgcttgctgtcagtctagtggaaacacttagcttgcaatgtgggtgtatgcctggataagtcgtatggatgctggccatgatgaattggatgcagtttaggacgtatagtggaaatcttgccagacacggttgatctcaccctccggggggctatagagatcgctggaacactaccggacaaccggaacattgcagaaatgcagcc

SEQ ID NO: 58 - RNA, 368 nt, Artichoke mottled crinkle virus
  source 1..368: mol_type = genomic RNA
gacagactcttcagtctgattctgtggaaatgagagtaattctgacatagcatacaggttactcgtgttgggttctagatgttatgatgacgagctggtcgggctccgcacgggtttggtcgcccggggatggagatatggaaagggtctcgtgtgctgttagtcagtcgaaagacgtgcttgcaacatgggcctataaccagataagtcatagcaatactagccaacatgaattggattcctgtttacgaaagttaggtgtcacttgtggagacagagttagacacggttgatctcacccttcgggggggctatagagatcactggaagcactaccggacaaccggaacattgcagcaatgcagccc

SEQ ID NO: 59 - RNA, 371 nt, Pelargonium necrotic spot virus
  source 1..371: mol_type = genomic RNA
gacagactcttcagtctgatgttacggagatgagtagaacaaagtgagggactatgggttaccctttgtagatttggtccgcatgatgacgagttaggtcaggccccgcgccaggtttggtcacctgggggatggggacacggaaaggatctcgcatgctgttagtctgacgaaagtcgagcttgcaatgtgggtctatgcctaaataagtcgtgtgggttgccgcctacaaggaattggatgccacttttctctcagtgagctctctcattgaagtagcattagacacggttgatctcacccttcgggggggctatagagatcgctggaagcactaccggacaaaccggaacattgcagaaattcagccc

SEQ ID NO: 60 - RNA, 369 nt, Grapevine Algerian latent virus
  source 1..369: mol_type = genomic RNA
gacagactcttcagtctgagttggtggatgtgagtgtactttgtttgagcatacaggttactcttagtatgattaggtctacgtgatgacgagtcaggtcaggctccgcactgggtttggtcgcccaggggatggagacacggaaaggacatcgtgtgctataagtctgacgtaagtcgagcttgcaacatgggtctatgcctggataagtcacgtggttgcccctactatgaattggatgcctgtttacgactagcaaacgacactcgtggaaactaatttggacacggttgatctcacccttcgggggggctatagagatcgctggaagcactaccggacaaccggaacattgcagaaatgcagccc

SEQ ID NO: 61 - RNA, 370 nt, Pelargonium leaf curl virus
  source 1..370: mol_type = genomic RNA
gacagactcttcagtctgagtttgctgaaatgagtgtaaatctggcatagcatacaggttactctcgttgggttctagatgtcatgatgacgagtcggtcgggctccgcactaggtttggtcgcctaggggatggagatatggaaagggtctcgtgtgctgttagtcagtcggaagacgtgcttgcaacatgggcctataaccggataagtcatgacaatactagccaacgtgaattggattcctgtttacgaaagttaggtgtcacttgtggaaacaaacctagacacggttgatctcacccttcgggggggctatagagatcgctggaagcactaccggacaaccggaacattgcagaaatgcagccc

SEQ ID NO: 62 - RNA, 369 nt, Pear latent virus
  source 1..369: mol_type = genomic RNA
gacagactcttcacgcttggagtgaagtttggtggcaactatacaccttagactactggttactactgtcaggttggaacccacattaattgagctaggtcgggccccgtcgtaggattggtcacctacggaatggggatatggaaaggatctcgtaccctgtgagtcaaacgaaagttgtgcttgggatatggatctatgcctagtaggttatgtggtgtgtctctgactttaattggatgccagttttcaacaggagtggaagtaactgaacaacactttgagcacggttgatctcaccctccgggggggctatagagatcgctggaaacactaccggacaaccggaacattgcagaaatgcagccc

SEQ ID NO: 63 - RNA, 369 nt, Lisianthus necrosis virus
  source 1..369: mol_type = genomic RNA
gacagactcttcacgcttggagtgaagtttggtggcagctatacaccttagaccactggttactactgtcaggttggaacccacactaattgagctaggccgggccccgtcgtaggtttggtcacctacggaatggggatatggaaaggatctcgtaccctgtgagtcaaacgaaagttgtgcttgggatatgggtctatgcctagtaggttatgtggtgtgtctctgactttaattggatgccagttttcaacaggagtggcagcgactgaacaacactttgagcacggttgatctcatccttcgggggggctatagagatcaccggaagcactaccggacaaccggaacattgcagaaatgcagccc

SEQ ID NO: 64 - RNA, 105 nt, Turnip yellow mosaic virus
  source 1..105: mol_type = genomic RNA
gttctcgatctttaaaatcgttagctcgccagttagcgaggtctgtccccacacgacagataatcgggtgcaactcccgcccctcttccgagggtcatcggaacc

SEQ ID NO: 65 - RNA, 443 nt, Red clover necrotic mosaic virus; identical to SEQ ID NO: 14
  source 1..443: mol_type = genomic RNA

SEQ ID NO: 66 - RNA, 146 nt, Turnip crinkle virus
  source 1..146: mol_type = genomic RNA
aacggtggcagcactgtctagctgcgggcattagactggaaaactagtgctctttgggtaaccactaaaatcccgaaagggtgggctgtggtgaccttccgaactaaaagatagcctccctcctcgcgcggggggggggcctgccc

SEQ ID NO: 67 - RNA, 112 nt, Cardamine chlorotic fleck virus
  source 1..112: mol_type = genomic RNA
ggtgtggaaaccagagagtcttgcggtaaccaacgaaatctcttcggagtgggtgttggggaccaccaaatgaagaaaatagcccctccctcgcgcaggggggggccagccc

SEQ ID NO: 68 - RNA, 165 nt, Cucurbit aphid-borne yellows virus
  source 1..165: mol_type = genomic RNA
ccgctctgtggagacgagcgtgactccacccggcatccagtgggcccgaccaaatcactgatgacatcaagccaaagatgtaaaattggaacgactccgaaaggataggcaacgaacgttcccaccttagtggaaacagggggactccccctggcatttcggtgt

SEQ ID NO: 69 - RNA, 531 nt, Citrus yellow vein-associated virus
  source 1..531: mol_type = genomic RNA
atagggtcattggtttaccgatgatacctgttcagaataggattgctcgagcttcgttggttagggtaactcacataccttcttccataactggaaaaggtcgtgtgagcaacctaaccagttaatgtaggtgtctttccgtatctagtcacgatggtaagcaacccgtttatctgtacggcgctcacccgtgggtaggaaggtgaaggttttgtgtcctttaggtcttggacagtctgcgggcttgggaacgacgccccgctagcaacgtactgctctcctaccggactggtagcttaattgtcatcttggagcgatagcactgtgggcctcacccttcgcgcgttggacgtgttgcgtgccccccacagatttgtgaaactctatggagcagttccgcgagccagaagggaggatggccgcctggcgtaatccaggagctctggggggcttgtactcagagtagcattctgctttagactgttaactttatgaaccacgcgtgtcacgtggggagagttaacagcgccc

SEQ ID NO: 70 - RNA, 39 nt, Opuntia umbra-like virus
  source 1..39: mol_type = genomic RNA
gggtaattatggatttaaatgaaacccatggttactcgt

SEQ ID NO: 71 - RNA, 34 nt, Fig umbra-like virus
  source 1..34: mol_type = genomic RNA
aaatatggattcgatatcaatgcccaaagcctgt

SEQ ID NO: 72 - RNA, 159 nt, Mus musculus
  source 1..159: mol_type = other RNA
gctgaaggtgttcctggagaacgtgatccgcgacgccgtcacctacacggagcacgccaagcgcaagaccgtcaccgccatggacgtggtctacgcgctcaagcgccagggccgcactctctacggattcggcggttaatcgactaacaaacgattttc

SEQ ID NO: 73 - RNA, 460 nt, synthetic construct
  source 1..460: mol_type = other RNA
aaaaaagtcagtaccatcacggaattattctcgcttatggcctgataaaaaagtcagtaccatcacggaattattctcgcttatggcctgataaaaaagtcagtaccatcacggatttattctcgcttatggcctgataaaaaagtcagtaccatcacggaattattctcgcttatggcctgataaaaaattcagtaccatcacggaattattctcgcttatggcctgataaaaaagtcagtaccatcacggaattattctcgcttatggcctgataaaaaagtcagtaccatcacggaattattctcgcttatggcctgataaaaaagtcagtaccatcacggaattattctcgcttatggcctgataaaaaagtcagtaccatcacggaattattctcgcttatggcctgataaaaaagtcagtaccatcacggaattattctcgcttatggcctgat

SEQ ID NO: 74 - RNA, 460 nt, synthetic construct
  source 1..460: mol_type = other RNA
aaaaaaactcactatttgttttcgcgcccagttgcaaaaagtgtcgaaaaaaactcactatttgttttcgcgcccagttgcaaaaagtgtcgaaaaaaactcactatttgttttcgcgcccagttgcaaaaagtgtcgaaaaaaactcactatttgttttcgcgcccagttgcaaaaagtgtcgaaaaaaactcactatttgttttcgcgcccagttgcaaaaagtgtcgaaaaaaactcactatttgttttcgcgcccagttgcaaaaagtgtcgaaaaaaactcactatttgttttcgcgcccagttgcaaaaagtgtcgaaaaaaactcactatttgttttcgcgcccagttgcaaaaagtgtcgaaaaaaactcactatttgttttcgcgcccagttgcaaaaagtgtcgaaaaaaactcactatttgttttcgcgcccagttgcaaaaagtgtcg

SEQ ID NO: 75 - RNA, 82 nt, Homo sapiens
  source 1..82: mol_type = other RNA
cacacaaagcaacaaaagaacgaagcaacaaaagcatagatttattgaaatgaaagtacattctacagagtgggggcaggct

SEQ ID NO: 76 - RNA, 97 nt, Homo sapiens
  source 1..97: mol_type = other RNA
gagaaatgcaaatcaaaaccacaatggaataccatctcacgccagtcagaatggcaattattaaaaaatcacaacaattaatgatggcaaggctgtg

SEQ ID NO: 77 - RNA, 81 nt, Homo sapiens
  source 1..81: mol_type = other RNA
aacaatcaatggaagcagaaacaaataaaccaaggtgtgcatcaaggaatacattcacgcatgatggctgtatgagtaaaa

SEQ ID NO: 78 - RNA, 84 nt, Homo sapiens
  source 1..84: mol_type = other RNA
gctcaaggaaataaaataggacacaaagaaatggaaaaacattccatactcatggatagaaagaatcaatatcatgaaatggcc

SEQ ID NO: 79 - RNA, 90 nt, Homo sapiens
  source 1..90: mol_type = other RNA
gcaataaccaaacaacctcattaaaaagtaggcaaaggacataaacagacacttttcaaaagaagacatacacgtggccaacaaacatat

SEQ ID NO: 80 - RNA, 87 nt, Homo sapiens
  source 1..87: mol_type = other RNA
gacttaaacataagacctaaaaccataaaaaccacagaagaaaacataggcaatgccattcaggacataggcatgggcaaagacttc

SEQ ID NO: 81 - RNA, 102 nt, Homo sapiens
  source 1..102: mol_type = other RNA
tacaaagaactcaaacaaatcagcaagaacaaaaacaatcccaacaaaatgttggacaaagacatgaatagacaattctcgaaagaagatgtacaaatggct

SEQ ID NO: 82 - RNA, 86 nt, Homo sapiens
  source 1..86: mol_type = other RNA
gacagtcagacaatcacaaagaaacaagaatgaaaatgaatgaacaaaaccttcaagaaatatgggattatgaagaggccaaatgt

SEQ ID NO: 83 - RNA, 85 nt, Homo sapiens
  source 1..85: mol_type = other RNA
ggacaaattgctagaaataaacaaattaccaaaaatgattcaagtagagacagagaatcaaaatagaactacacataagtgggcc

SEQ ID NO: 84 - RNA, 84 nt, Homo sapiens
  source 1..84: mol_type = other RNA
ggaaataacagagaacacaaacaaatgggaaaacattccatgttcatggataggaagaatcaatattgtgaaaatggccatact

SEQ ID NO: 85 - RNA, 85 nt, Homo sapiens
  source 1..85: mol_type = other RNA
gcaaagaaagaatgaagcagcaaaagaacgaaagcaggaatttattgaaaaccaaagtacactccacagtatgggagcggacccg

SEQ ID NO: 86 - RNA, 103 nt, Homo sapiens
  source 1..103: mol_type = other RNA
ggaatttaagcaaatcaacaagcaaaaccaaaataatcccattaaaaagtgggtaaaggacatgaatacacacttgtcaatagaggacattcaagtggccaac

SEQ ID NO: 87 - RNA, 78 nt, Homo sapiens
  source 1..78: mol_type = other RNA
tcagtctagatcttaaaggaacaccagagggagtatttaaatgtgcccaataagcaagaattatggtgatgtggaagt

SEQ ID NO: 88 - RNA, 96 nt, Homo sapiens
  source 1..96: mol_type = other RNA
cagaccataaataaacacagaagacacacgagtgtaaagtcagtgccccgctgcgaattaaatcggggtgatgtgatggcgagtgagtgggtagtt

SEQ ID NO: 89 - RNA, 71 nt, synthetic construct
  source 1..71: mol_type = other RNA
gaaatggaatcgaagagaatggaaacaaatggaatggaattgaatggaatggaattgaatggaatgggaac

SEQ ID NO: 90 - RNA, 75 nt, Homo sapiens
  source 1..75: mol_type = other RNA
aggaaatacaagacaacaaacacagaaacacaaccatcgggcatcatgaaacctcgtgaagataatcatcagggt

SEQ ID NO: 91 - RNA, 94 nt, synthetic construct
  source 1..94: mol_type = other RNA
gaaaggattcgaatggaatgaaaaagaattgaatggaatagaacagaatggaatcaaatcgaatgaaatggaatggaatagaaaggaatggaat

SEQ ID NO: 92 - RNA, 89 nt, Homo sapiens
  source 1..89: mol_type = other RNA
gaaagaatcaagaggaaatgcaagaaatccaaaacactgtaacagatatgatgaataatgaggtatgcactcatcagcagactcgacat

SEQ ID NO: 93 - RNA, 89 nt, Homo sapiens
  source 1..89: mol_type = other RNA
tcaaaagaaaagcaacctaacaaatacgggaagaatatttgaatagacatttcacaggaaaagatatatgaatggccaaaaagcaaatg

SEQ ID NO: 94 - RNA, 96 nt, Homo sapiens
  source 1..96: mol_type = other RNA
cagttcaaaaattattgcaacaaaatgagagagatgagtttatcttgcaaactaatggatggtagcagtgacagtggcaaaacgtggtttgattct

SEQ ID NO: 95 - RNA, 67 nt, synthetic construct
  source 1..67: mol_type = other RNA
gtaatggaatggaatggaaaggaatcgaaacgaaaggaatggagacagatggaatggaatggaacag

SEQ ID NO: 96 - RNA, 88 nt, Homo sapiens
  source 1..88: mol_type = other RNA
tatacgcaaatcaataaatgtaatccagcatataaacagtactaaagacaaaaaccacatgattatctcaatagatgcagaaaaggcc

SEQ ID NO: 97 - RNA, 102 nt, Homo sapiens
  source 1..102: mol_type = other RNA
gaccgaaacaacaacagaaacagaaacaaacaacaataagaaaaaatgttaagcaaaacaaatgattgcacaacttacatgattactgagtgttctaatggt

SEQ ID NO: 98 - RNA, 72 nt, Homo sapiens
  source 1..72: mol_type = other RNA
ccactgctcaaggaaataagagagaacacaaacaaatgaaaaaacattccatgctcatggataggaagaatc

SEQ ID NO: 99 - RNA, 67 nt, synthetic construct
  source 1..67: mol_type = other RNA
gcaatcgaatggaatggaatcgaacggaatggaataaaatggaagaaaactggcaagaaatggaatc

SEQ ID NO: 100 - RNA, 71 nt, synthetic construct
  source 1..71: mol_type = other RNA
gaaatcatcatcaaacggaatcgaatggaatcattgaatggaatggaatggaatcatcatggaatggaaac

SEQ ID NO: 101 - RNA, 79 nt, Homo sapiens
  source 1..79: mol_type = other RNA
tgttgagagaaattaaacaaagcacagataaatggaaaaacgtgttcatagattgaaagacttcatgttgtatggtgtc

SEQ ID NO: 102 - RNA, 73 nt, Homo sapiens
  source 1..73: mol_type = other RNA
ccaacataagacaaagaaacatccagcagctgcctatggcaaaagattacaatgtgtcaaacaagagggcaat

SEQ ID NO: 103 - RNA, 89 nt, Homo sapiens
  source 1..89: mol_type = other RNA
cccaacttcaaattatactacaaggctacagtaatcaaaaaagcatagtactattacaaaaacagacacacaggccaatggaatacaat

SEQ ID NO: 104 - RNA, 123 nt, Encephalomyocarditis virus
  source 1..123: mol_type = genomic RNA
tagtgtagtcactggcacaacgcgttacccggtaagccaatcgggtatacacggtcgtcatactgcagacagggttcttctactttgcaagatagtctagagtagtaaaataaatagatagag

SEQ ID NO: 105 - RNA, 99 nt, Coxsackievirus B3
  source 1..99: mol_type = genomic RNA
attagagacaatttgaaataatttagattggcttaaccctactgtgctaaccgaaccagataacggtacagtaggggtaaattctccgcattcggtgcg

SEQ ID NO: 106 - RNA, 233 nt, Hepatitis C virus subtype 1b
  source 1..233: mol_type = genomic RNA
acggggggctagtcactccaggccaataggccattctgtttttttttttttttttttttttttttttttttttttttttttttttttttttttttttttttttttcctttttcttcttccttttcttctttctttggtggctccatcttagccctagtcacggctagctgtgaaaggtccgtgagccgcatgactgcagagagtgctgatactggcctctctgcagatcatgt

SEQ ID NO: 107 - RNA, 236 nt, Recombinant Hepatitis C virus
  source 1..236: mol_type = genomic RNA
agcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgt

SEQ ID NO: 108 - RNA, 139 nt, Simian rotavirus
  source 1..139: mol_type = genomic RNA
ggaccaagctaacaacttggtatccaactttggtgagtatgtagctatatcaagctgtttgaactctgtaagtaaggatgcgtatacgcattcgctacacagagtaatcactcagatggtatagtgagaggatgtgacc

SEQ ID NO: 109 - RNA, 301 nt, synthetic construct
  source 1..301: mol_type = other RNA
tgcccctgctcggagagcccctgactgggttaaagtcacaggccccttgtctcaggtagagaccctgtccaggtaggacactttggctaaggttaaaagcttgttgaatcagtacaataactgatagtcgtggttgacacgcagacctcttacaagagtgtctaggtgcctttgagagttactctttgctctcttcggaagaacccttaggggttcgtgcatgggcttgcatagcaagtcttagaatgcgggtaccgtacagtgttgaaaaacactgtaaatctctaaaagagaccagcaa

SEQ ID NO: 110 - RNA, 602 nt, Woodchuck hepatitis virus
  source 1..602: mol_type = genomic RNA
taatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtttcgcctc

SEQ ID NO: 111 - RNA, 592 nt, Woodchuck hepatitis virus
  source 1..592: mol_type = genomic RNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 112 - RNA, 141 nt, synthetic construct
  source 1..141: mol_type = other RNA
gagtgaagattgaccatctcacaaaagctgttacgtgcttgtaacacactacgcgcccgttttgtattcgggaagtagttgcgaaaacggtccccttattgcctgacaagctaagggccacccttctttccccaccgccat

SEQ ID NO: 113 - RNA, 201 nt, Barley yellow dwarf virus; identical to SEQ ID NO: 1
  source 1..201: mol_type = genomic RNA

SEQ ID NO: 114 - RNA, 38 nt, Tobacco necrosis virus D
  source 1..38: mol_type = genomic RNA
gataccttaccagtatctcagtgattaagtaatcagct

SEQ ID NO: 115 - RNA, 305 nt, Tobacco necrosis virus D; identical to SEQ ID NO: 2
  source 1..305: mol_type = genomic RNA

SEQ ID NO: 116 - RNA, 63 nt, Turnip crinkle virus
  source 1..63: mol_type = genomic RNA
ggtaatctgcaaatccctggcacccgcctaaaattgccctcatcaaccttctctctattcacg

SEQ ID NO: 117 - RNA, 144 nt, Turnip crinkle virus
  source 1..144: mol_type = genomic RNA
cggtggcagcactgtctagctgcgggcattagactggaaaactagtgctctttgggtaaccactaaaatcccgaaagggtgggctgtggtgaccttccgaactaaaagatagcctccctcctcgcgcggggggggggcctgccc

SEQ ID NO: 118 - RNA, 99 nt, Dengue virus type 2
  source 1..99: mol_type = genomic RNA
gttgttagtctacgtggaccgacaaagacagattctttgagggagctaagctcaacgtagttctaacagttttttaattagagagcagatctctgcaca

SEQ ID NO: 119 - RNA, 95 nt, West Nile virus
  source 1..95: mol_type = genomic RNA
gtagttcgcctgtgtgagctgacaaacttagtagtgtttgtgaggattaacaacaattaacacagtgcgagctgtttcttggcacgaagatctcg

SEQ ID NO: 120 - RNA, 571 nt, West Nile virus
  source 1..571: mol_type = genomic RNA
gatagtattatagttagtttagtgtaaataggatttattgagaatggaagtcaggccagattaatgctgccaccggaagttgagtagacggtgctgcctgcggctcaaccccaggaggactgggtgaccaaagctgcgaggtgatccacgtaagccctcagaaccgtctcggaaggaggaccccacgtgctttagcctcaaagcccagtgtcagaccacactttaatgtgccactctgcggagagtgcagtctgcgatagtgccccaggtggactgggttaacaaaggcaaaacatcgccccacgcggccataaccctggctatggtgttaaccagggagaagggactagaggttagaggagaccccgcgtaaaaaagtgcacggcccaacttggctgaagctgtaagccaagggaaggactagaggttagaggagaccccgtgccaaaaacaccaaaagaaacagcatattgacacctgggatagactaggggatcttctgctctgcacaaccagccacacggcacagtgcgccgacataggtggctggtggtgctagaacacaggatct

SEQ ID NO: 121 - RNA, 143 nt, Soybean dwarf virus
  source 1..143: mol_type = genomic RNA
gtaaagttgacacctttacagaagtgatcttgcttgtaagagttaattcatcaagagttaatacaagatccacctcccggcaccttcgtatcgtgtttgaggtatctctagtgtttggttgtttgaatactaggtaatactta

SEQ ID NO: 122 - RNA, 655 nt, Soybean dwarf virus
  source 1..655: mol_type = genomic RNA
tatagtagaagtaagtgtgcgctttccatcacattgaattggagccaaccttggtgagttgctgtcgtgggatcctgggaaacaggttcggtgaacaaaaagccggtaatctcgggcgttcgtcagacgccgtcgtatcaacacagtatgcatatccatcaataacccaattttcttagtgactaaaaattagtgttagtttttccgcctctcttgtaaaccacatacaagatgcgagcttgtggagagtaccatcccttgaataaggatggttgaaagtttctggtcccaatccattgatcaaaattggcactgcttctggtgactacactgccgctggcgatacagcgtaaacaatttctacttcttgttcgatgaaacgagaacaattagtaaagtcttgccgttcacatgtctatctgacggtactaggcttcgtccctctgatgacgttaaatgaaagcggagtaaaccgacatcaccttactaggttgggttaacctagggaaaccgggaaaatgcccgggacacttggtttagtggattttaaactcgagggctcttcctcgttaaaaagggggtagccaggttgaaaactggcactagggagcgatccctctttgttgttaaggtggctttgtgtccacctgccccc

SEQ ID NO: 123 - RNA, 93 nt, Turnip yellow mosaic virus
  source 1..93: mol_type = genomic RNA
gtaatcaactaccaattccagctctcttttgacaactggtcttatacccacttccgtacacttgcaaccctcgtaagacaattgcaaatgagt

SEQ ID NO: 124 - RNA, 105 nt, Turnip yellow mosaic virus; identical to SEQ ID NO: 64
  source 1..105: mol_type = genomic RNA

SEQ ID NO: 125 - RNA, 105 nt, Zika virus
  source 1..105: mol_type = genomic RNA
gttgttgatctgtgtgaatcagactgcgacagttcgagtttgaagcgaaagctagcaacagtatcaacaggttttattttggatttggaaacgagagtttctggt

SEQ ID NO: 126 - RNA, 428 nt, Zika virus
  source 1..428: mol_type = genomic RNA
gcaccaatcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccaccctttaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtct

SEQ ID NO: 127 - RNA, 72 nt, Homarus americanus
  source 1..72: mol_type = mRNA
ccaaggcaggcccacggtcctagcgttctcagttcttttttgttggttgggaaactcctaaaaaaccgccac

SEQ ID NO: 128 - RNA, 66 nt, synthetic construct
  source 1..66: mol_type = other RNA
gtatttttacaacaattaccaacaacaacaaacaacaaacaacattacaattactatttacaatta

SEQ ID NO: 129 - RNA, 217 nt, Tobacco mosaic virus
  source 1..217: mol_type = genomic RNA
gtcctgcaacttgaggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggccc

SEQ ID NO: 130 - RNA, 89 nt, Pea enation mosaic virus-2
  source 1..89: mol_type = genomic RNA
gggtatttatagagatcagtatgaactgtgtcgctaggatcaagcggtggttcacacctgacttcacccctggcgagggcgtgaagtct

SEQ ID NO: 131 - RNA, 34 nt, synthetic construct
  source 1..34: mol_type = other RNA
aaaaaaagtaaagacaggaaactttactgactaa

SEQ ID NO: 132 - RNA, 622 nt, Satellite tobacco mosaic virus; identical to SEQ ID NO: 23
  source 1..622: mol_type = genomic RNA

SEQ ID NO: 133 - RNA, 106 nt, Blackcurrant reversion virus
  source 1..106: mol_type = genomic RNA
tagcgatgtgcacgtttaggagataaatagctagtatctttctactcggattcccctctgtcgtcatagaggggtttcaagggatgtcatcatcagcgttggctat

SEQ ID NO: 134 - RNA, 116 nt, Yellow fever virus
  source 1..116: mol_type = genomic RNA
gtaaatcctgtgtgctaattgaggtgcattggtctgcaaatcgagttgctaggcaataaacacatttggattaattttaatcgttcgttgagcgattagcagagaactgaccagaa

SEQ ID NO: 135 - RNA, 506 nt, Yellow fever virus
  source 1..506: mol_type = genomic RNA
caccatctaacaggaataaccgggatacaaaccacgggtggagaaccggactccccacaacctgaaaccgggatataaaccacggctggagaaccggactccgcacttaaaatgaaacagaaaccgggataaaaactacggatggagaaccggactccacacattgagacagaagaagttgtcagcccagaactccacacgagttttgccactgctaagctgtgaggcagtgcaggctgggacagccgacctccaggttgcgaaaaacctggtttctgggacctcccaccccagagtaaaaagaacggagcctccgctaccaccctcccacgtggtggtagaaagacggggtctagaggttagaggagaccctccagggaacaaatagtgggaccatattgacgccagggaaagaccggagtggttctctgcttttcctccagaggtctgtgagcacagtttgctcaagaataagcagacctttggatgacaaacacaaaaccact

SEQ ID NO: 136 - RNA, 167 nt, Cardiovirus C1
  source 1..167: mol_type = genomic RNA
atggattggatttcggttgagcccccacccggtacaacgctttaccttagaagccactaaggtgtacgcggtcatcggggacccctcctggcctttggtttattggtgaattactagttcagttaggttttgttagttaggaaaaaaaaaaaaaaaaaaaaaaaaaa

SEQ ID NO: 137 - RNA, 237 nt, Aichivirus A1
  source 1..237: mol_type = genomic RNA
tagttagggaacacagtgcctcggcacgcttagttagttagaaccctttgcccactcctccatggtgatataaagaccacccacttccttcgggtgagcccctaagccatggttgtactgcactatcatcctaagacggtccttcttcggatcgcaatctcaccctggtgccgcgcttccttcgggaactgcacccgcggaccagggccgtctttgaacttttctaactgttcttac

SEQ ID NO: 138 - RNA, 124 nt, Saffold virus
  source 1..124: mol_type = genomic RNA
acatcctctcgatcggatcgcaacgtgttacccaggaatccacttgggtgtacgcggccgttctgacgttggaattctgtagatgaaagttagctaggagcttttaattggaaatgagaacaaa

SEQ ID NO: 139 - RNA, 64 nt, Homo sapiens
  source 1..64: mol_type = mRNA
cctcgccccggacctgccctcccgccaggtgcacccacctgcaataaatgcagcgaagccggga

SEQ ID NO: 140 - RNA, 128 nt, Homo sapiens
  source 1..128: mol_type = mRNA
cctcgccccggacctgccctcccgccaggtgcacccacctgcaataaatgcagcgaagccgggacctcgccccggacctgccctcccgccaggtgcacccacctgcaataaatgcagcgaagccggga

SEQ ID NO: 141 - RNA, 153 nt, Rabbit picornavirus
  source 1..153: mol_type = genomic RNA
ggggcgcgtgcgcacccgcgtgtgaacagtcgcccttccgccacctgggcgtggcctccatcagagctactcttcctgagtgctttctttttcctcttttcttttctttcttcttttcttttctgtttctgtgtgtagagagtgaatgcagca

SEQ ID NO: 142 - RNA, 174 nt, Aichivirus B
  source 1..174: mol_type = genomic RNA
cctcagaactctggcctcccactgttcgtcctggtagaatagacaagcttttcactatccctaagaccactctctccgtggcattttcctctcagtgtcctgctccctcacgggactgcacccaggaactgtgagcttccccccctgtccttgaaatttctattcttttcttgc

SEQ ID NO: 143 - RNA, 170 nt, Porcine kobuvirus
  source 1..170: mol_type = genomic RNA
gacaccagatacactcttgctctgtatcgtccctgtgtagaatagactcatgctttcctaagaccggatctctcttccggcactctcacccgcctgccctgccttcatcttgaagctgcacccagggaggcggtccctgactgtcctttactttctattctgtattgcaa

SEQ ID NO: 144 - RNA, 178 nt, Aichivirus D
  source 1..178: mol_type = genomic RNA
agagacaggtctcctgtgtccctctgcgttctctctggtttcctgtaagttaggctctttgtaatggtgttggtctctattgcataaaattcttcctcctttgctctccctccacctctcgggggggggggcccgcccttttcttcctaacttgaaaaaaaaaaaaaaaaaaaaaaaa

SEQ ID NO: 145 - RNA, 163 nt, Alfalfa mosaic virus
  source 1..163: mol_type = genomic RNA
attagtcattggtaattcaatgccaacctccactgggtgggttaaggttgaggtatagaatcctattcgctcctgataggagaaattctatattgcttatatacgtgcttatgcacgtatataaatgctcatgctaaattgcatgaatgcccctaagggatgc

SEQ ID NO: 146 - RNA, 47 nt, synthetic construct
  source 1..47: mol_type = other RNA
aataaacaataaaccaataaaccaataaacccaataaacaataaacc

SEQ ID NO: 147 - RNA, 46 nt, synthetic construct
  source 1..46: mol_type = other RNA
agtgtaaagtgtaaaagtgtaaaagtgtaaaaagtgtaaagtgtaa

SEQ ID NO: 148 - RNA, 45 nt, synthetic construct
  source 1..45: mol_type = other RNA
tcgatgatcgatgaatcgatgatcgatgaaatcgatgatcgatga

SEQ ID NO: 149 - RNA, 45 nt, synthetic construct
  source 1..45: mol_type = other RNA
ttgccgattgccgaattgccgattgccgaaattgccgattgccga

SEQ ID NO: 150 - RNA, 46 nt, synthetic construct
  source 1..46: mol_type = other RNA
atgccgaatgccgaaatgccgaaatgccgaaaatgccgaatgccga

SEQ ID NO: 151 - RNA, 42 nt, synthetic construct
  source 1..42: mol_type = other RNA
cgccgctcgccgcttcgccgctttcgccgcttttcgccgctt

SEQ ID NO: 152 - RNA, 42 nt, synthetic construct
  source 1..42: mol_type = other RNA
cggggctcggggcttcggggctttcggggcttttcggggctt

SEQ ID NO: 153 - RNA, 42 nt, synthetic construct
  source 1..42: mol_type = other RNA
gccgccgccgctttgccgccgccgcttttgccgccgccgctt

SEQ ID NO: 154 - RNA, 42 nt, synthetic construct
  source 1..42: mol_type = other RNA
ggcggccgcctttggcggccgccttttggcggccgccttttt

SEQ ID NO: 155 - RNA, 42 nt, synthetic construct
  source 1..42: mol_type = other RNA
ccccagcacctttccccagcaccttttccccagcaccttttt

SEQ ID NO: 156 - RNA, 42 nt, synthetic construct
  source 1..42: mol_type = other RNA
gaagaagaagtgaagaagaagttgaagaagaagtttgaagaa

SEQ ID NO: 157 - RNA, 42 nt, synthetic construct
  source 1..42: mol_type = other RNA
ccagccgcacccagccgcacccagccgcacccagccgcaccc

SEQ ID NO: 158 - RNA, 42 nt, synthetic construct
  source 1..42: mol_type = other RNA
ccccagcacctttccccagcaccgaagaagaagtttgaagaa

SEQ ID NO: 159 - RNA, 45 nt, synthetic construct
  source 1..45: mol_type = other RNA
gaagaagaagtgaagaagaagttccccagcacctttccccagcac

SEQ ID NO: 160 - RNA, 53 nt, synthetic construct
  source 1..53: mol_type = other RNA
cgcatatatcgatcagcgcaggtagagaagctacatcaccagcatgcgagaga

SEQ ID NO: 161 - RNA, 50 nt, synthetic construct
  source 1..50: mol_type = other RNA
ggagtgctagaggatgaccttaaatcagggacgatattataattgactaa

SEQ ID NO: 162 - RNA, 54 nt, Homo sapiens
  source 1..54: mol_type = other RNA
gttcctcctgcccgctgggcctcccaacgggccctcctcccctccttgcaccgg

SEQ ID NO: 163 - RNA, 200 nt, Homo sapiens
  source 1..200: mol_type = mRNA
tgaattataaactaattgattaatgtccccaaagaaatctgctttctactatatctttcagcattagagatttttctgttcttgaaaatacagtctgtgctctttgatttttgctattgtacggtttcatgcatttttttaaagggcatttgaggggaggattattgctatgaatgaaaaaaatattttagcttagacta

SEQ ID NO: 164 - RNA, 364 nt, Homo sapiens
  source 1..364: mol_type = mRNA
aaagcctcttcctgatgatcccaactcagaattcactgtttaccaaacaccttggtcataataatgtcattagtttctccatttttattttctgaactgtacattcacaacttatgtttctttgagattaatagatattgggggaaaaacgcctttttaggaaaattatagtgaaaatttgacagttgattggcataatttcttgtttgaatgctgcctccattatataggtccttccaggaactcaaacactgtaagtgaaatatgggagtatagtttttattatttcttcttttccttttgttttcataatataatgcagtttgttcaggaaatcagcacaaagcctgatagtactttacta

SEQ ID NO: 165 - RNA, 198 nt, Homo sapiens
  source 1..198: mol_type = mRNA
agaaccagcggaactcaaaactgctgttcaagaaacctcttcaagacttttgacttagaacctgctatattatcaagcttacctactgttatctctaaaattttttttgtgttaatgtaaagttgaatttctaggaaacgtgcctttgttttttaatatgcactccaaattagaaggccggccccgtccacattttgc

SEQ ID NO: 166 - RNA, 200 nt, Homo sapiens
  source 1..200: mol_type = mRNA
atgtattgatggaatttctggctttctaaatgattttactttctgccttgaattttcaaggcatagatgtcaacttacagaataacatgttttaagataattaagtttaaaccagagaatttgattgttactcattttgctctcatgttctaaacagcaacagtgtaactagtcttttgttgtaaatggttattttcctt

SEQ ID NO: 167 - RNA, 450 nt, Homo sapiens
  source 1..450: mol_type = other RNA
aaacaatggtgtgttgtttgaaaaccagctgcttcaaattggacttaagtctgaatttcggcagaatttaggtcggatgtttatcttttatggtaataagacctccacgcagttcctaaactttaccccaacactaatctgttcagacgaccttcagcctaacctgaacctgcagaccaagcccgtggacccgaccgtggaggggggcgcgcaggtgcagcaggtggtcaacatagagtgcgtgtccgacttcacggaggcgccagtcctcaacattcagttcaggtatgggggcaccttccagaacgtgtctgtgcagctgcccatcactctcaacaaattcttccagccgacagaaatggcttctcaggatttctttcaacgttggaagcagttgagcaatccacagcaggaagtgcagaacatcttcaaagcaaagcacccaatgga

SEQ ID NO: 168 - RNA, 500 nt, Homo sapiens
  source 1..500: mol_type = mRNA
tttcttctagagctcatgtagaggaccacgttttgaccaaaacttctcctaaggcatttgactccatctatatttcacaaaagagacttcctttccccaagaattatcatggaatgtcagatgttactttgttaccaacactgttatatttctacattgaaatgcaaagtggaactaggagtttggaatgcattaagagcagacaagcttggtcataatagatccagtgtttttcagattcctttcactgccttaatctttgcaacagggtggaagtttttttcttccctcaaaattttcatggacatgcaatcttatctaaaagcctgcttcagggctgggcgcggtggctaacacctataattcccagcactttgggaggccaagatgggcaaatcacttgagtctaggagttcaagaccagcctggccaatatggcaaaaccctgtctctactaaaaatataaaatcagccaggcatggtggcgcacacctataatc

SEQ ID NO: 169 - RNA, 500 nt, Homo sapiens
  source 1..500: mol_type = mRNA
agagctgggggcggatggtggcccaggagacagcacaccaccaggctccacacgtgcatgctttccccaagagggaatggactgtacattgctcatttcacactcttcagaagacatttcatacctgccctggtcctgcttgaaggtttgtccaggcagagcagctcctgcagcgcctcggtctgtgacagtcctcctagcacccccatggctttgagcctcggggactcatcaagtccaagaaaagagggaggggtggcagaggatctgcagccctggccccgcggtgcatgaggctgggtgcagttctaaacctacattctcgatttttcttaagccaaaaatgaatgctaactcctttgccagtaaaattctgggaaacagggactgaggccacacatcatttccagtcatctgtgtgtttttaaggccagccacttgtccctgttgaggcctggctatggaactaaatacagtgttggtcttgcctgtccttcaaa

SEQ ID NO: 170 - RNA, 160 nt, Human adenovirus 2
  source 1..160: mol_type = genomic RNA
gggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctcctttt

SEQ ID NO: 171 - RNA, 366 nt, Homo sapiens
  source 1..366: mol_type = other RNA
gacactaaagaaggtgtgggccagacaaggccttggagcccacacatgggatcaagtcctgcaagcagaggtggcgatttgttaaagggccaaaagtatccaaggttagggttggagcaggggtgctgggacctggggcactgtgggactgggacacttttatgttaatgctctggacttgcctccagagactgctccagagttggtgacacagctgtcccaagggcccctctgtatctagcctggaaccaaggttatcttggaactaaatgacttttctcctctcagtgggtggtagcagagggatcaagcagttatttgatttgtgctcacttttgatatggccaataaaaccataccgactga

SEQ ID NO: 172 - RNA, 145 nt, Homo sapiens
  source 1..145: mol_type = mRNA
ggaggatggggcaggacaggacgcggccaccccaggcctttccagagcaaacctggagaagattcacaatagacaggccaagaaacccggtgcttcctccagagccgtttaaagctgatatgaggaaataaagagtgaactggaa

SEQ ID NO: 173 - RNA, 293 nt, synthetic construct
  source 1..293: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagcta

SEQ ID NO: 174 - RNA, 170 nt, Homo sapiens
  source 1..170: mol_type = mRNA
ggccaggccccttccctctgcccccgggtgcttgaagtctagccccatcctggtccaatgcgctcttggtagcctcctttcccagctgcccgcccgccgccatgccgcccttactgcccctgcgcctgtgccggctgtggccccgcaaccctccctcccggctcctcgga

SEQ ID NO: 175 - RNA, 149 nt, synthetic construct
  source 1..149: mol_type = other RNA
ttccagccagacacccgccccccggccctggctaagaagttgcttcctgttgccagcatgacctaccctcgcctctttgatgccatccgctgccacctccttttgctcctggaccctttagcctctctgcccttccactctctgacccc

SEQ ID NO: 176 - RNA, 136 nt, synthetic construct
  source 1..136: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 177 - RNA, 234 nt, synthetic construct
  source 1..234: mol_type = other RNA
ctttgcaggatgaaacacttccccgcttggctctcattcttccacaagagagacctttctccggacctggttgctactggttcagcaactctgcagaaaatgtcctcccctgtggctgcctcagctcatgccctttggcctgaagtcccagcattgatggcagcccctcatcttccaagttttgtgctcccctttacctaacgcttcctgcctcccatgcatctgtactcctcc

SEQ ID NO: 178 - RNA, 142 nt, synthetic construct
  source 1..142: mol_type = other RNA
caagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 179 - RNA, 337 nt, synthetic construct
  source 1..337: mol_type = other RNA
aaaatttaaaaaaaactgagacaaatgatggagtcttgatcgtgaacactagcaagtaccccaatatgtatgttcctgtcggtgctgtgactgaacagggatatctaaatctcggtgggcgccaaactgctcgtactctaatgtacaactttccaaccagagcaggacagtgtggtggagtcatcacatgtactgggaaagtcatcgggatgcatgttggtgggaacggttcacacgggtttgcagcggccctgaagcgatcatacttcactcagagtcaaggtgaaatccagtggatgagaccttcgaaggaagtggattaaaaaaaaataaaaaa

SEQ ID NO: 180 - RNA, 255 nt, synthetic construct
  source 1..255: mol_type = other RNA
aaatttaaaaaaaactgagacaaatgatggagtcttgatcgtgaacactagcaagtaccccaatatgtatgttcctgtcggtgctgtgactgaacagggatatctaaatctcggtgaaaacatcgggatgcatgttggtgggaacggttcacacgggtttgcagcggccctgaagcgatcatacttcactcagagtcaaggtgaaatccagtggatgagaccttcgaaggaagtggattaaaaaaaaataaaaaa

SEQ ID NO: 181 - RNA, 157 nt, Homo sapiens
  source 1..157: mol_type = mRNA
gcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaac

SEQ ID NO: 182 - RNA, 337 nt, Homo sapiens
  source 1..337: mol_type = mRNA
aattcagacaaggcccacagaataagattttccatgcatttgcaaatacgtatattctttttccatccacttgcacaatatcattaccatcactttttcatcattcctcagctactactcacattcatttaatggtttctgtaaacatttttaagacagttgggatgtcacttaacattttttttttttgagctaaagtcagggaatcaagccatgcttaatatttaacaatcacttatatgtgtgtcgaagagtttgttttgtttgtcatgtattggtacaagcagatacagtataaactcacaaacacagatttgaaaataatgcacatatggtg

SEQ ID NO: 183 - RNA, 98 nt, Hepacivirus macronycteridis
  source 1..98: mol_type = genomic RNA
ttcgggcaaggcatgcgagataaaaagggtctcgtatgagggcgtggcaacccctcccccccagctgcggcggcacaaaagcgtctcgcgtgtcgtcg

SEQ ID NO: 184 - RNA, 741 nt, synthetic construct
  source 1..741: mol_type = other RNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaac

SEQ ID NO: 185 - RNA, 749 nt, synthetic construct
  source 1..749: mol_type = other RNA
ttaaaacagctgtgggttgtcacccacccacagggtccactgggcgctagtacactggtatctcggtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcggccacc

SEQ ID NO: 186 - protein, 20 aa, Homo sapiens
MYRMQLLSCIALSLALVTNS

SEQ ID NO: 187 - protein, 18 aa, synthetic construct
MGVKVLFALICIAVAEAK

SEQ ID NO: 188 - protein, 28 aa, Homo sapiens
MGVHECPAWLWLLLSLLSLPLGLPVLGA

SEQ ID NO: 189 - protein, 13 aa, synthetic construct
MFVFLVLLPLVSS

SEQ ID NO: 190 - protein, 8 aa, synthetic construct
  VARIANT 2: note = I
  VARIANT 4: note = X is any amino acid
DVEXNPGP

SEQ ID NO: 191 - protein, 6 aa, synthetic construct
CHYSEL

SEQ ID NO: 192 - protein, 9 aa, synthetic construct
  VARIANT 1: note = X is absent or G or H
  VARIANT 2: note = X is absent or D or G
  VARIANT 3: note = X is absent or D or V or I or S or M
  VARIANT 5: note = X is any amino acid
XXXEXNPGP

SEQ ID NO: 193 - protein, 9 aa, synthetic construct
GDVESNPGP

SEQ ID NO: 194 - protein, 9 aa, synthetic construct
GDIEENPGP

SEQ ID NO: 195 - protein, 7 aa, synthetic construct
VEPNPGP

SEQ ID NO: 196 - protein, 7 aa, synthetic construct
IETNPGP

SEQ ID NO: 197 - protein, 9 aa, synthetic construct
GDIESNPGP

SEQ ID NO: 198 - protein, 9 aa, synthetic construct
GDVELNPGP

SEQ ID NO: 199 - protein, 9 aa, synthetic construct
GDIETNPGP

SEQ ID NO: 200 - protein, 8 aa, synthetic construct
GDVENPGP

SEQ ID NO: 201 - protein, 9 aa, synthetic construct
GDVEENPGP

SEQ ID NO: 202 - protein, 9 aa, synthetic construct
GDVEQNPGP

SEQ ID NO: 203 - protein, 7 aa, synthetic construct
IESNPGP

SEQ ID NO: 204 - protein, 9 aa, synthetic construct
GDIELNPGP

SEQ ID NO: 205 - protein, 9 aa, synthetic construct
HDIETNPGP

SEQ ID NO: 206 - protein, 9 aa, synthetic construct
HDVETNPGP

SEQ ID NO: 207 - protein, 9 aa, synthetic construct
HDVEMNPGP

SEQ ID NO: 208 - protein, 9 aa, synthetic construct
GDMESNPGP

SEQ ID NO: 209 - protein, 9 aa, synthetic construct
GDVETNPGP

SEQ ID NO: 210 - protein, 9 aa, synthetic construct
GDIEQNPGP

SEQ ID NO: 211 - protein, 8 aa, synthetic construct
DSEFNPGP

SEQ ID NO: 212 - protein, 22 aa, synthetic construct
GSGATNFSLLKQAGDVEENPGP

SEQ ID NO: 213 - protein, 5 aa, synthetic construct
GRLRR

SEQ ID NO: 214 - skipped

SEQ ID NO: 215 - DNA, 131 bp, synthetic construct
  source 1..131: mol_type = other DNA
aacaacagataacttacagctagtcggaaggtgcagagactcgacgggagctaccctaacgtcaagacgagggtaaagagagagtccaattctcaaagccaataggcagtagcgaaagctgcgggagaatg

SEQ ID NO: 216 - DNA, 118 bp, synthetic construct
  source 1..118: mol_type = other DNA
aaataattgagccttagagaagaaattctttaagtggatgctctcaaactcagggaaacctaaatctagctatagacaaggcaatcctgagccaagccgaagtagtaattagtaagtt

SEQ ID NO: 217 - DNA, 178 bp, synthetic construct
  source 1..178: mol_type = other DNA
cttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcg

SEQ ID NO: 218 - DNA, 235 bp, synthetic construct
  source 1..235: mol_type = other DNA
aaatagcaatatttacctttggagggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagat

SEQ ID NO: 219 - DNA, 167 bp, synthetic construct
  source 1..167: mol_type = other DNA
ggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatg

SEQ ID NO: 220 - DNA, 93 bp, synthetic construct
  source 1..93: mol_type = other DNA
taattgaggcctgagtataaggtgacttatacttgtaatctatctaaacggggaacctctctagtagacaatcccgtgctaaattgtaggact

SEQ ID NO: 221 - DNA, 182 bp, synthetic construct
  source 1..182: mol_type = other DNA
taaacaactaacagctttagaaggtgcagagactagacgggagctaccctaacggattcagccgagggtaaagggatagtccaattctcaacatcgcgattgttgatggcagcgaaagttgcagagagaatgaaaatccgctgactgtaaaggtcgtgagggttcgagtccctccgccccca

SEQ ID NO: 222 - DNA, 128 bp, synthetic construct
  source 1..128: mol_type = other DNA
acggtagacgcagcggacttagaaaactgggcctcgatcgcgaaagggatcgagtggcagctctcaaactcagggaaacctaaaactttaaacattmaagtcatggcaatcctgagccaagctaaagc

SEQ ID NO: 223 - DNA, 210 bp, synthetic construct
  source 1..210: mol_type = other DNA
ttaaactcaaaatttaaaatcccaaattcaaaattccgggaaggtgcagagactcgacgggagctaccctaacgtaaagccgagggtaaagggagagtccaattctcaaagcctgaagttgctgaagcaacaaggcagtagtgaaagctgcgagagaatgaaaatccgttgactgtaaaaagtcgtgggggttcaagtccccccaccccc

SEQ ID NO: 224 - DNA, 151 bp, synthetic construct
  source 1..151: mol_type = other DNA
atggtagacgctacggacttagaaaactgagccttgatagagaaatcttttaagtggaagctctcaaattcagggaaacctaaatctgaatacagatatggcaatcctgagccaagcccagaaaatttagacttgagatttgattttggag

SEQ ID NO: 225 - DNA, 207 bp, synthetic construct
  source 1..207: mol_type = other DNA
ggctttcaatttgaaatcagaaattcaaaattcagggaaggtgcagagactcgacgggagctaccctaacgtaaaggcgagggtaaagggagagtccaattcttaaagcctgaagttgtgcaagcaacaaggcaacagtgaaagctgtggaagaatgaaaatccgttgaccttaaacggtcgtgggggttcaagtccccccaccccc

SEQ ID NO: 226 - DNA, 147 bp, synthetic construct
  source 1..147: mol_type = other DNA
atggtagacgctacggacttagaaaactgagccttgatagagaaatctttcaagtggaagctctcaaattcagggaaacctaaatctgaatacagatatggcaatcctgagccaagcccggaaattttagaatcaagattttatttt

SEQ ID NO: 227 - DNA, 178 bp, synthetic construct
  source 1..178: mol_type = other DNA
agaaatggagaaggtgtagagactggaaggcaggcaccctaacgttaaaggcgagggtgaagggacagtccagaccacaaaccagtaaatctgggcagcgaaagctgtagatggtaagcataacccgaaggtcagtggttcaaatccacttcccgccaccaaattaaaaaaacaataa

SEQ ID NO: 228 - DNA, 178 bp, synthetic construct; identical to SEQ ID NO: 227
  source 1..178: mol_type = other DNA

SEQ ID NO: 229 - DNA, 189 bp, synthetic construct
  source 1..189: mol_type = other DNA
acaacagataacttactaacttacagctagtcggaaggtgcagagactcgacgggagctaccctaacgtcaagacgagggtaaagagagagtccaattctcaaagccaataggcagtagcgaaagctgcgggagaatgaaaatccgtagcgtctaaacggtcgtgtgggttcaagtccctccaccccca

SEQ ID NO: 230 - DNA, 141 bp, synthetic construct
  source 1..141: mol_type = other DNA
agacgctacggacttaaataattgagccttagagaagaaattctttaagtggatgctctcaaactcagggaaacctaaatctagctatagacaaggcaatcctgagccaagccgaagtagtaattagtaagttagtaagtt

SEQ ID NO: 231 - DNA, 186 bp, synthetic construct
  source 1..186: mol_type = other DNA
aacaacagataacttactagttactagtcggaaggtgcagagactcgacgggagctaccctaacgtcaagacgagggtaaagagagagtccaattctcaaagccaataggcagtagcgaaagctgcgggagaatgaaaatccgtagcgtctaaacggtcgtgtgggttcaagtccctccaccccca

SEQ ID NO: 232 - DNA, 133 bp, synthetic construct
  source 1..133: mol_type = other DNA
agacgctacggacttaaataattgagccttagagaagaaattctttaagtggatgctctcaaactcagggaaacctaaatctagctatagacaaggcaatcctgagccaagccgaagtagtaattagtaagtt

SEQ ID NO: 233 - DNA, 160 bp, synthetic construct
  source 1..160: mol_type = other DNA
ttgcaaaacaaggttcaacgactagtcttcggacgtagggtcaagcgactcgaaatggggagaatccctccgggattgtgatatagtctggactgcatggtaacatgcagcagttcataagagaacgggttgagaattagcgagctcaatcgaacatacg

SEQ ID NO: 234 - skipped

SEQ ID NO: 235 - DNA, 90 bp, synthetic construct
  source 1..90: mol_type = other DNA
aaaatgcgcctttaaacggtaacgtttatcgaaaactcctttaattgctggaaagtcctttatggaaaactagcagccaaggttttgctt

SEQ ID NO: 236 - skipped

SEQ ID NO: 237 - DNA, 226 bp, synthetic construct
  source 1..226: mol_type = other DNA
cagtagctgtaaatgcccaacgactatccctgatgaatgtaagggagtagggtcaagcgacccgaaacggcagacaactctaagagttgaagatatagtctgaactgcatggtgacatgcagctgtttatcctcgtataaatatgaatacgaggtgaaacgatgaaatgaattacattgtttcatataaacgggtagagaagtagcgaactctactgaacacattg

SEQ ID NO: 238 - skipped

SEQ ID NO: 239 - DNA, 74 bp, synthetic construct
  source 1..74: mol_type = other DNA
taacgtaagtcaagctcatgtaaaatctgcctaaaacgggaaactctcactgagacaatccgttgctaaatcag

SEQ ID NO: 240 - skipped

SEQ ID NO: 241 - DNA, 50 bp, synthetic construct
  source 1..50: mol_type = other DNA
gtacctttaacttccataagaacatggaaatcatggaaggtaatgccaag

SEQ ID NO: 242 - skipped

SEQ ID NO: 243 - DNA, 51 bp, synthetic construct
  source 1..51: mol_type = other DNA
gtacctttaacttccaaaagatacataaaaatcatggaaggtaatgccaag

SEQ ID NO: 244 - skipped

SEQ ID NO: 245 - DNA, 20 bp, synthetic construct
  source 1..20: mol_type = other DNA
tttttatgtatcttttgcgt

SEQ ID NO: 246 - skipped

SEQ ID NO: 247 - DNA, 50 bp, synthetic construct
  source 1..50: mol_type = other DNA
atgaagtgaacacgttattcagttcaaacggacagactccttttgtaaca

SEQ ID NO: 248 - skipped

SEQ ID NO: 249 - DNA, 50 bp, synthetic construct
  source 1..50: mol_type = other DNA
atgaagtgaacacgttacataagcttggaatgcagactccttttgtaaca

SEQ ID NO: 250 - skipped

SEQ ID NO: 251 - DNA, 20 bp, synthetic construct
  source 1..20: mol_type = other DNA
tgcattccaagcttatgagt

SEQ ID NO: 252 - skipped

SEQ ID NO: 253 - DNA, 136 bp, synthetic construct
  source 1..136: mol_type = other DNA
aaaaaagggactcactatttgttttcgcgcccagttgcaaaaagtgtcgccgcatctagagggccaaaaaagggactcactatttgttttcgcgcccagttgcaaaaagtgtcgccgcatctagagggccaaaaaa

SEQ ID NO: 254 - DNA, 152 bp, synthetic construct
  source 1..152: mol_type = other DNA
aaaaaaccgatcactaaaacagctcgtagtccgtttttcttattgtctgcggttgagtcgctgaacaccaaccaaaaaaccgatcactaaaacagctcgtagtccgtttttcttattgtctgcggttgagtcgctgaacaccaaccaaaaaa

SEQ ID NO: 255 - DNA, 98 bp, synthetic construct
  source 1..98: mol_type = other DNA
aaaaaaactcactatttgttttcgcgcccagttgcaaaaagtgtcgaaaaaaactcactatttgttttcgcgcccagttgcaaaaagtgtcgaaaaaa

SEQ ID NO: 256 - DNA, 144 bp, synthetic construct
  source 1..144: mol_type = other DNA
aaaaaagtcagtaccatcacggaattattctcgcttatggcctgataaaaaagtcagtaccatcacggaattattctcgcttatggcctgataaaaaagtcagtaccatcacggaattattctcgcttatggcctgataaaaaa

SEQ ID NO: 257 - DNA, 4443 bp, Homo sapiens
  source 1..4443: mol_type = genomic DNA
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttag

SEQ ID NO: 258 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 259 - skipped

SEQ ID NO: 260 - DNA, 15 bp, synthetic construct
  source 1..15: mol_type = other DNA
gatatcgccgcgctc

SEQ ID NO: 261 - DNA, 21 bp, synthetic construct
  source 1..21: mol_type = other DNA
cctgaatgcggctaatcctaa

SEQ ID NO: 262 - DNA, 20 bp, synthetic construct
  source 1..20: mol_type = other DNA
aacacggacacccaaagtag

SEQ ID NO: 263 - skipped

SEQ ID NO: 264 - DNA, 15 bp, synthetic construct
  source 1..15: mol_type = other DNA
taacgggcaactctg

SEQ ID NO: 265 - RNA, 95 nt, Bat pegivirus
  source 1..95: mol_type = genomic RNA
acttggcaaggcaccccctggctagtggctgcacttaactgagtggccggctagtcggtgggtgggggcacgtgactcccccttccccacaccgg

SEQ ID NO: 266 - RNA, 50 nt, Tamana bat virus
  source 1..50: mol_type = genomic RNA
ggcaaggtacggattagccgtaggggcttgagaaccccccctccccactc

SEQ ID NO: 267 - DNA, 4446 bp, synthetic construct
  source 1..4446: mol_type = other DNA
atgcagcggtccccgctcgaaaaggccagtgtcgtgtccaaactcttcttctcatggactcggcctatccttagaaaggggtatcggcagaggcttgagttgtctgacatctaccagatcccctcggtagattcggcggataacctctcggagaagctcgaacgggaatgggaccgcgaactcgcgtctaagaaaaacccgaagctcatcaacgcactgagaaggtgcttcttctggcggttcatgttctacggtatcttcttgtatctcggggaggtcacaaaagcagtccaacccctgttgttgggtcgcattatcgcctcgtacgaccccgataacaaagaagaacggagcatcgcgatctacctcgggatcggactgtgtttgcttttcatcgtcagaacacttttgttgcatccagcaatcttcggcctccatcacatcggtatgcagatgcgaatcgctatgtttagcttgatctacaaaaagacactgaaactctcgtcgcgggtgttggataagatttccatcggtcagttggtgtccctgcttagtaataacctcaacaaattcgatgagggactggcgctggcacatttcgtgtggattgccccgttgcaagtcgcccttttgatgggccttatttgggagctgttgcaggcatctgccttttgtggcctgggatttctgattgtgttggcattgtttcaggctgggcttgggcggatgatgatgaagtatcgcgaccagagagcgggtaaaatctcggaaagactcgtcatcacttcggaaatgatcgaaaacatccagtcggtcaaagcctattgctgggaagaagctatggagaagatgattgaaaacctccgccaaactgagctgaaactgacccgcaaggcggcgtatgtccggtatttcaattcgtcagcgttcttcttttccgggttcttcgttgtctttctctcggttttgccttatgccttgattaaggggattatcctccgcaagattttcaccacgatttcgttctgcattgtattgcgcatggcagtgacacggcaatttccgtgggccgtgcagacatggtatgactcgcttggagcgatcaacaaaatccaagacttcttgcaaaagcaagagtacaagaccctggagtacaatcttactactacggaggtagtaatggagaatgtgacggctttttgggaagagggttttggagaactgtttgagaaagcaaagcagaataacaacaaccgcaagacctcaaatggggacgattccctgtttttctcgaacttctccctgctcggaacacccgtgttgaaggacatcaatttcaagattgagaggggacagcttctcgcggtagcgggaagcactggtgcgggaaaaactagcctcttgatggtgattatgggggagcttgagcccagcgaggggaagattaaacactccgggcgtatctcattctgtagccagttttcatggatcatgcccggaaccattaaagagaacatcattttcggagtatcctatgatgagtaccgatacagatcggtcattaaggcgtgccagttggaagaggacatttctaagttcgccgagaaggataacatcgtcttgggagaagggggtattacattgtcgggagggcagcgagcgcggatcagcctcgcgagagcggtatacaaagatgcagatttgtatctgcttgattcaccgtttggatacctcgacgtattgacagaaaaagaaatcttcgagtcgtgcgtgtgtaaacttatggctaataagacgagaatcctggtgacatcaaaaatggaacaccttaagaaggcggacaagatcctgatcctccacgaaggatcgtcctacttttacggcactttctcagagttgcaaaacttgcagccggacttctcaagcaaactcatggggtgtgactcattcgaccagttcagcgcggaacggcggaactcgatcttgacggaaacgctgcaccgattctcgcttgagggtgatgccccggtatcgtggaccgagacaaagaagcagtcgtttaagcagacaggagaatttggtgagaaaagaaagaacagtatcttgaatcctattaactcaattcgcaagttctcaatcgtccagaaaactccactgcagatgaatggaattgaagaggattcggacgaacccctggagcgcaggcttagcctcgtgccggattcagagcaaggggaggccattcttccccggatttcggtgatttcaaccggacctacacttcaggcgaggcgaaggcaatccgtgctcaacctcatgacgcattcggtaaaccaggggcaaaacattcaccgcaaaacgacggcctcaacgagaaaagtgtcacttgcaccccaggcgaatttgactgaactcgacatctacagccgtaggctttcgcaagaaaccggacttgagatcagcgaagaaatcaatgaagaagatttgaaagagtgtttctttgatgacatggaatcaatcccagcggtgacaacgtggaacacatacttgcgttacatcacggtgcacaagtccttgattttcgtcctcatctggtgtctcgtgatctttctcgctgaggtcgcagcgtcacttgtggtcctctggctgcttggtaatacgcccttgcaagacaaaggcaattctacacactcaagaaacaattcctatgccgtgattatcacttctacaagctcgtattacgtgttttacatctacgtaggagtggccgacactctgctcgcgatgggtttcttccgaggactcccactcgttcacacgcttatcactgtctccaagattctccaccataagatgcttcatagcgtactgcaggctcccatgtccaccttgaatacgctcaaggcgggaggtattttgaatcgcttctcaaaagatattgcaattttggatgaccttctgcccctgacgatcttcgacttcatccagttgttgctgatcgtgattggggctattgcagtagtcgctgtcctccagccttacatttttgtcgcgaccgttccggtgatcgtggcgtttatcatgctgcgggcctatttcttgcagacgtcacagcagcttaagcaactggagtctgaagggaggtcgcctatctttacgcatcttgtgaccagtttgaagggattgtggacgttgcgcgcctttggcaggcagccctactttgaaacactgttccacaaagcgctgaatctccatacggcaaattggtttttgtatttgagtaccctccgatggtttcagatgcgcattgagatgatttttgtgatcttctttatcgcggtgacttttatctccatcttgaccacgggagagggcgagggacgggtcggtattatcctgacactcgccatgaacattatgagcactttgcagtgggcagtgaacagctcgattgatgtggatagcctgatgaggtccgtttcgagggtctttaagttcatcgacatgccgacggagggaaagcccacaaaaagtacgaaaccctataagaatgggcaattgagtaaggtaatgatcatcgagaacagtcacgtgaagaaggatgacatctggcctagcgggggtcagatgaccgtgaaggacctgacggcaaaatacaccgagggagggaacgcaatccttgaaaacatctcgttcagcattagccccggtcagcgtgtggggttgctcgggaggaccgggtcaggaaaatcgacgttgctgtcggccttcttgagacttctgaatacagagggtgagatccagatcgacggcgtttcgtgggatagcatcaccttgcagcagtggcggaaagcgtttggagtaatcccccaaaaggtctttatctttagcggaaccttccgaaagaatctcgatccttatgaacagtggtcagatcaagagatttggaaagtcgcggacgaggttggccttcggagtgtaatcgagcagtttccgggaaaactcgactttgtccttgtagatgggggatgcgtcctgtcgcatgggcacaagcagctcatgtgcctggcgcgatccgtcctctctaaagcgaaaattcttctcttggatgaaccttcggcccatctggacccggtaacgtatcagatcatcagaaggacacttaagcaggcgtttgccgactgcacggtgattctctgtgagcatcgtatcgaggccatgctcgaatgccagcaatttcttgtcatcgaagagaataaggtccgccagtacgactccatccagaagctgcttaatgagagatcattgttccggcaggcgatttcaccatccgatagggtgaaactttttccacacagaaattcgtcgaagtgcaagtccaaaccgcagatcgcggccttgaaagaagagactgaagaagaagttcaagacacgcgtctttgataa

SEQ ID NO: 268 - DNA, 4440 bp, synthetic construct
  source 1..4440: mol_type = other DNA
atgcagcggagccccctggagaaggccagcgtggtcagcaaactgtttttcagctggacccggcccatcctgcggaagggctatcggcaacggctggagctgagcgacatctaccagatccccagcgtggacagcgccgacaacctgagcgagaagctggagcgggagtgggaccgggagctggccagcaagaaaaaccccaagctgatcaacgccctgcggaggtgcttcttctggcggttcatgttctacggcatctttctgtatctgggcgaggtgaccaaggccgtgcagcctctgctgctgggacggattattgccagctacgaccccgacaacaaggaggaacggagtattgccatctacctgggcatcggcctgtgcctgttgttcatcgtgcggaccctgctgctgcaccccgccatcttcggcctgcaccatatcggcatgcaaatgcggattgccatgttcagcctgatctacaagaaaaccctcaagctgagctctcgggtcctggacaagatcagcatcggccagctggtgagcctgctcagcaacaatctgaacaagttcgacgagggcctggccctggcccacttcgtgtggatcgctcccctccaggtggctctgctcatgggcctgatctgggagctgttgcaggctagcgctttctgcggcctgggctttctgattgtgctggccctgtttcaggctggcctcggccggatgatgatgaagtaccgggaccagcgggccggcaaaatcagtgagcgcctggtcatcacaagcgaaatgattgagaatatccagagcgtgaaggcctactgttgggaagaggccatggagaagatgatcgagaacctgcggcagaccgaactgaaactgacccggaaggccgcttacgtgcggtacttcaacagcagcgccttctttttcagtggcttcttcgtggtgttcctgagcgtgctgccctacgccctgatcaaaggcattatcctccggaagatcttcaccacaattagcttttgcatcgtgctgcggatggccgtgacccggcagttcccctgggccgtgcagacctggtacgacagcctgggcgccatcaacaagatccaggatttcctccagaagcaggagtacaagaccctggagtacaacctgaccaccactgaggtggttatggagaacgtgaccgccttctgggaggagggcttcggcgagctgttcgagaaggccaagcagaacaacaacaatcggaaaaccagcaacggcgacgatagcctgttctttagcaacttcagcctgctgggcacccccgtgctgaaggacatcaacttcaagatcgagcggggccagctgctcgccgtggccggcagcaccggcgccggcaagaccagcctgctgatggtgatcatgggcgagctggagcccagcgagggcaagatcaagcacagcggccggatcagcttctgcagccagttcagctggatcatgcccggcaccatcaaagagaatatcatatttggcgtcagctacgacgagtaccggtaccgcagcgtcatcaaggcctgccagctggaggaggacatcagcaagttcgccgagaaggacaacatcgtgctgggcgagggcggaatcaccctgagtggcggacagcgcgcccgcatcagtctggctcgggccgtgtacaaggacgccgacctgtacctgctggacagccccttcggctacctggacgtgctgaccgagaaggagatcttcgagagctgcgtgtgtaagctcatggccaacaagacccggattctcgtcacaagtaagatggagcacctgaagaaggccgacaagatcctgatcctgcacgagggcagctcttacttctacggcaccttcagcgagctgcagaacctgcagcccgacttcagcagcaagctgatgggctgcgacagcttcgaccagttcagcgccgagcggcgcaacagtatcctcaccgaaacactgcatcggtttagcctggagggcgacgcccccgtgagctggaccgaaacaaagaagcagagcttcaagcagaccggcgagttcggcgagaagcggaagaatagcattctgaaccccatcaacagcatccggaagttcagcatcgtgcagaagacacccctccagatgaacggcatcgaggaggacagcgacgagcccctggagcggaggctgagcctggtgcccgacagcgagcagggcgaggccatcctgccccggatcagcgtgatcagcaccggccccaccctgcaggcccggcggcggcaaagcgtcctgaacctgatgacccacagcgtgaaccagggccagaacatccaccggaagaccaccgccagcacccggaaggtgagcctggccccccaggccaacctgaccgagctggacatctacagccggagactgagccaggaaacaggcctggagatcagcgaggaaatcaacgaggaggacctgaaggagtgcttcttcgacgacatggagagcatccccgccgtgaccacctggaacacctacctgcggtacatcaccgtgcacaagagcctcatctttgtgctgatctggtgcctggtgatcttcctggccgaggtggccgccagcctggtggtgctgtggctgttgggcaacacccccctgcaggacaagggcaacagcacccacagccggaacaacagctacgccgtcatcattaccagcaccagcagctactacgtgttctacatctacgtgggcgtggccgacaccctgctggccatgggcttcttccggggcctgcccctggtgcacaccctgatcaccgtgagtaagattctgcaccacaagatgctgcacagcgtgctgcaggcccctatgagtaccctcaacaccctgaaggccggcgggatcctcaaccgcttcagcaaggatattgccattctggacgacctgctccccctgaccatcttcgacttcatccagctgctcctgatcgtgatcggcgctattgccgtggtggccgtgctgcagccctacatcttcgtggccaccgtgcccgtgatcgtggccttcatcatgctgcgggcctacttcctgcagaccagccagcaactgaagcagctggagagtgagggacggagccccatcttcacccacctggtgaccagcctgaagggcctgtggaccctgcgggccttcggccggcagccctacttcgaaacactgttccacaaggccctgaacctgcacaccgccaactggttcctgtacctcagcacactgcggtggttccagatgcggatcgagatgatcttcgtgatcttctttattgccgtgaccttcatcagcatcctgaccacaggcgagggcgagggccgggtgggcatcatcctgaccctggccatgaacatcatgagcacactgcagtgggccgtgaacagctccatcgacgtggacagcctgatgcggagtgtgagccgggtgttcaagttcatcgacatgcccaccgagggcaagcccaccaagagcaccaagccctacaagaacggccaactgagtaaggtgatgatcattgagaacagccacgtgaagaaggacgacatctggcccagcggcggccagatgaccgtgaaggacctgaccgccaagtataccgaaggcggcaacgccatcctggagaacatcagcttcagcatcagccccggccagcgggtgggcctgttgggccggaccggcagcggcaagagcaccctgctgagcgccttcctgcggctgctgaacaccgagggcgagatccagatcgacggcgtgagctgggacagcatcaccctgcagcagtggcggaaggccttcggcgtgatcccccagaaggtgttcatcttcagcggcaccttccggaagaatctggatccctacgagcagtggagcgaccaggagatctggaaggtggccgacgaggtgggcctgcggagcgtgatcgagcagttccccggcaagctggacttcgtgctggtggacggcggctgtgtgctcagccacggccacaagcagctgatgtgcctggcccggagcgtgctgagcaaggccaagatcctgctcctggacgagcccagcgcccacctggaccccgtgacctaccagatcatccggcggaccctgaagcaggccttcgccgactgcaccgtgatcctgtgcgagcaccggatcgaggccatgctggagtgccagcagttcctggtgatcgaggagaacaaggtgcggcagtacgacagcatccagaagctgttgaacgagcggagcctgttccggcaggccatcagccccagcgaccgggtgaagctgttcccccaccggaacagcagcaagtgcaagagcaagccccagatcgccgcactgaaggaggaaacagaggaagaggtgcaggacacccggctg

SEQ ID NO: 269 - DNA, 17 bp, synthetic construct
  source 1..17: mol_type = other DNA
ccttgcacatgccggag

SEQ ID NO: 270 - DNA, 17 bp, synthetic construct
  source 1..17: mol_type = other DNA
acagagcctcgcctttg

SEQ ID NO: 271 - RNA, 96 nt, synthetic construct
  source 1..96: mol_type = other RNA
ggctcatctcgacaagaggcggcagtcctcagtactcttactcttttctgtaaagaggagactgctggactcgccgcccaagttcgagcatgagcc

SEQ ID NO: 272 - RNA, 74 nt, synthetic construct
  source 1..74: mol_type = other RNA
ggctagaggcggcagtcctcagtactcttactcttttctgtaaagaggagactgctggactcgccgcccgagcc

SEQ ID NO: 273 - DNA, 47 bp, synthetic construct
  source 1..47: mol_type = other DNA
aataaacaataaaccaataaaccaataaacccaataaacaataaacc

SEQ ID NO: 274 - DNA, 46 bp, synthetic construct
  source 1..46: mol_type = other DNA
agtgtaaagtgtaaaagtgtaaaagtgtaaaaagtgtaaagtgtaa

SEQ ID NO: 275 - DNA, 45 bp, synthetic construct
  source 1..45: mol_type = other DNA
tcgatgatcgatgaatcgatgatcgatgaaatcgatgatcgatga

SEQ ID NO: 276 - DNA, 45 bp, synthetic construct
  source 1..45: mol_type = other DNA
ttgccgattgccgaattgccgattgccgaaattgccgattgccga

SEQ ID NO: 277 - DNA, 46 bp, synthetic construct
  source 1..46: mol_type = other DNA
atgccgaatgccgaaatgccgaaatgccgaaaatgccgaatgccga

SEQ ID NO: 278 - DNA, 42 bp, synthetic construct
  source 1..42: mol_type = other DNA
cggcgctcggcgcttcggcgctttcggcgcttttcggcgctt

SEQ ID NO: 279 - DNA, 42 bp, synthetic construct
  source 1..42: mol_type = other DNA
cgccgctcgccgcttcgccgctttcgccgcttttcgccgctt

SEQ ID NO: 280 - DNA, 42 bp, synthetic construct
  source 1..42: mol_type = other DNA
cggggctcggggcttcggggctttcggggcttttcggggctt

SEQ ID NO: 281 - DNA, 42 bp, synthetic construct
  source 1..42: mol_type = other DNA
gccgccgccgctttgccgccgccgcttttgccgccgccgctt

SEQ ID NO: 282 - DNA, 42 bp, synthetic construct
  source 1..42: mol_type = other DNA
ggcggccgcctttggcggccgccttttggcggccgccttttt

SEQ ID NO: 283 - DNA, 42 bp, synthetic construct
  source 1..42: mol_type = other DNA
ccccagcacctttccccagcaccttttccccagcaccttttt

SEQ ID NO: 284 - RNA, 42 nt, synthetic construct; identical to SEQ ID NO: 156
  source 1..42: mol_type = other RNA

SEQ ID NO: 285 - RNA, 42 nt, synthetic construct; identical to SEQ ID NO: 157
  source 1..42: mol_type = other RNA

SEQ ID NO: 286 - DNA, 42 bp, synthetic construct
  source 1..42: mol_type = other DNA
ccccagcacctttccccagcaccgaagaagaagtttgaagaa

SEQ ID NO: 287 - DNA, 45 bp, synthetic construct
  source 1..45: mol_type = other DNA
gaagaagaagtgaagaagaagttccccagcacctttccccagcac

SEQ ID NO: 288 - DNA, 64 bp, Homo sapiens
  source 1..64: mol_type = genomic DNA
cctcgccccggacctgccctcccgccaggtgcacccacctgcaataaatgcagcgaagccggga

SEQ ID NO: 289 - DNA, 128 bp, Homo sapiens
  source 1..128: mol_type = genomic DNA
cctcgccccggacctgccctcccgccaggtgcacccacctgcaataaatgcagcgaagccgggacctcgccccggacctgccctcccgccaggtgcacccacctgcaataaatgcagcgaagccggga

SEQ ID NO: 290 - DNA, 169 bp, Homo sapiens
  source 1..169: mol_type = genomic DNA
atactcccggccagggcctgtcccagttatgtgccttccctcaagccaaagccgaagcccctttccttaaggccctggtttgtcccgaaggggcctagtgttcccagcactgtgcctgctctcaagagcacttactgcctcgcaaataataaaaatttctagccagtca

SEQ ID NO: 291 - DNA, 256 bp, Homo sapiens
  source 1..256: mol_type = genomic DNA
cctctccggaggttccgctgcctccaagccggacttagggcttcaagaggcgggcgtgccctctggagtcccctaccatgactgaaggcgccagagactggcggtgtcttaagactccgggcaccgccacgcgctgtcaagcaacactctgcggacctgccggcgtagttgcagcgggggcttggggaggggctgggggttggacgggattgaggaaggtccgcacagcctgtctctgctcagttgcaataaacgt

SEQ ID NO: 292 - DNA, 73 bp, Homo sapiens
  source 1..73: mol_type = genomic DNA
aagtgttggttttctgccatcagtgaaaattcttaaacttggagcaacaataaaaagcttccagaacagatca

SEQ ID NO: 293 - DNA, 178 bp, Homo sapiens
  source 1..178: mol_type = genomic DNA
ccacacccccattcccccactccagataaagcttcagttatatctcacgtgtctggagttctttgccaagagggagaggctgaaatccccagccgcctcacctgcagctcagctccatcctacttgaaacctcacctgttcccaccgcattttctcctggcgttcgcctgctagtgtg

SEQ ID NO: 294 - DNA, 145 bp, synthetic construct
  source 1..145: mol_type = other DNA
gggtgggcgtcttcctgccccatgctcccctgccccccaccacctgcctgcttgcttctctggctgcctggtcagtgcagaggtgccccctgcagatgttcaataaaggagacaagtgctttcccagctcttttcctgcaccacc

SEQ ID NO: 295 - DNA, 205 bp, Homo sapiens
  source 1..205: mol_type = genomic DNA
agcccggcccacacccgacaccagccctgctgcttcctaacttattgcctgggcagtgcccaccatgcacccctgatgttcgccgtctggcgagcccttagccttgctgtagagacttccgtcacccttggtagagtttatttttttgatggctaagatactgctgatgctgaaataaactagggttttggcctgcctgcgtctg

SEQ ID NO: 296 - DNA, 116 bp, Homo sapiens
  source 1..116: mol_type = genomic DNA
ggctggcccagagctgtcaggaccatgaagccaggacagaggccaggagccagccctgcagccctccccacccggcatccacctgcatcccctctgggggcaggagcccaccccca

SEQ ID NO: 297 - DNA, 231 bp, Homo sapiens
  source 1..231: mol_type = genomic DNA
tagctgctccccatcccaaagtgggcctgggcagtcaggctccagggccctatgccaacctgcctttgttacaaggcagaggaagctttgtatttattggcttcaaggcccacctctctgtactctgggctctaaagttggaggtcaggttacctgaggtttgcaatttgcaacacccaccctccccccaatcagtgttcttatttcagtgacaataaaccatagagatga

SEQ ID NO: 298 - DNA, 102 bp, Homo sapiens
  source 1..102: mol_type = genomic DNA
gctccctccacacacctcaagccccatgccgtgctcatcctacccccaatccctccaataaacctgattctgctgcccagactatgtgctattcctaggctg

SEQ ID NO: 299 - DNA, 191 bp, Homo sapiens
  source 1..191: mol_type = genomic DNA
gagccctcgcgtcccaggccccagccagggggccggccttgtcccgcctcatccacagaaagggaggatgggcgatgacagttgtttctatgccttctgacccagtttcccagtttataactttatgacaatgagtttctcaagcccaaggccttatcttcaccccacaaacaataaagcaaagtaaagaa

SEQ ID NO: 300 - DNA, 35 bp, Homo sapiens
  source 1..35: mol_type = genomic DNA
cttcaggtgtttaataaagatgtgttgactcagcc

SEQ ID NO: 301 - DNA, 201 bp, Homo sapiens
  source 1..201: mol_type = genomic DNA
gtctggggcccgtgccagccggcagcccccaagcctgccccgctacccaagcagccccgccctcttcccctcaaccccggcccaggccgccctggccgcccgcctgtcactgcagccgcccctgccctgtgccgtgctgcgctcacctgcctccccagccagccgctgacctctcggctttcacttgggcagagggagcca

SEQ ID NO: 302 - DNA, 143 bp, Homo sapiens
  source 1..143: mol_type = genomic DNA
gagccttctgagcccagcgacttctgaagggccccttgcaaagtaatagggcttctgcctaagcctctccctccagccaataggcagctttcttaactatcctaacaagccttggaccaaatggaaataaagctttttgatgc

SEQ ID NO: 303 - skipped

SEQ ID NO: 304 - RNA, 22 nt, Homo sapiens
  source 1..22: mol_type = other RNA
acagacaaattagcctgctgga

SEQ ID NO: 305 - DNA, 278 bp, Homo sapiens
  source 1..278: mol_type = genomic DNA
gattgcccggaggaagcaaactcttcgtataaaaaaagcaggccatctgcttaacccttggctccaccataaggcactgggactcggatttctctatctgatagaggtattttctgtggccctgggagctgtctgtctttcccctacccccaaggatgccaggaagacgtccaccattagccatgtggcaacctttacttctatgcctcacaagtgcctttcagagagccccaattctgctttcccacaaaataaacctaatgccatcaggcaaaaca

SEQ ID NO: 306 - DNA, 272 bp, Homo sapiens
  source 1..272: mol_type = genomic DNA
gcacccatgcacccactcttcccagacctggggcgactcatggcgtgctcacctccagccgctggtggggggtgtcctgaaggacaggatgctctccccagacctccccagggtggtgaagctgtgccctggggccccagctaacccactcctctctctgtacgtcaatgtccctatttgaaagcagggatgatcccattccaccctgtccactctggatgagaattggccacctgatggttatttatacgtccagaagaacatcatttccc

SEQ ID NO: 307 - DNA, 57 bp, synthetic construct
  source 1..57: mol_type = other DNA
aatcagccagcccatggcccccaataaaggcagctgcctctgctccctctgaaccag

SEQ ID NO: 308 - DNA, 135 bp, Homo sapiens
  source 1..135: mol_type = genomic DNA
gattttagatattgttaatcctgccagtctttctcttcaagccagggtgcatcctcagaaacctactcaacacagcactctaggcagccactatcaatcaattgaagttgacactctgcattaaatctatttgcc

SEQ ID NO: 309 - DNA, 120 bp, Homo sapiens
  source 1..120: mol_type = genomic DNA
gagccgcagggacccacgctgtctgcgctggctccacccgggaacccgccgcacgctgtgttctaggcccgcccaccccaaccttctggtggggagaaataaacggtttagagactagga

SEQ ID NO: 310 - DNA, 268 bp, Homo sapiens
  source 1..268: mol_type = genomic DNA
aaacttcaggcagaacatccattcattcattcattggattgtatatcattgttgcacaatcagaattgataagcactgttcctccactccatttagcaattatgtcacccttttttattgcagttggtttttgaatgtctttcactccttttaaggataaactcctttatggtgtgactgtgtcttattcatctatacttgcagtgggtagatgtcaataaatgttacatacacaaataaataaaatgtttattccatggtaaattta

SEQ ID NO: 311 - DNA, 256 bp, Homo sapiens
  source 1..256: mol_type = genomic DNA
gtgctgcaggtagggccctcccaccccttcgccgcgccttacgaggcaagtctcccgcccttctctcagccaacatagaaaagcagcggagttcattctgctgtccggttgctgcacgggctccagcgcttcgcaactttggtttttttccacagatccagtggggtccggacactgggccccgcaggcgaaagcacgttccagccaccaggaggccacctattgtttcaaaataaagactgggttcctctcttgg

SEQ ID NO: 312 - DNA, 128 bp, Homo sapiens
  source 1..128: mol_type = genomic DNA
ggaagcattttcctgattgatgaaaaaaataactcagttatggccatctacccctgctagaaggttacagtgtattatgtagcatgcaatgtgttatgtagtgcttaataaaaataaaatgaaaaaaa

SEQ ID NO: 313 - DNA, 48 bp, Homo sapiens
  source 1..48: mol_type = genomic DNA
cccaccaccctggcctgctgtcctgcgtctatccatgtggaatgctgg

SEQ ID NO: 314 - DNA, 236 bp, synthetic construct
  source 1..236: mol_type = other DNA
caggccgtcctccacaaatcatatgtcccagataagcagccagcaggcagaaagatcagcttagctctgactgccagtgggtaaggccaagaaggagcctggcagacaaatggatggctgctggccttgaacatggcagagctgctgggtgtgccagatcaaaaagcagagagcaggccagcagcagcagactgttctcagcgctggagaaatgcattgctctgcccactacaccc

SEQ ID NO: 315 - DNA, 82 bp, Homo sapiens
  source 1..82: mol_type = genomic DNA
gactccagggttctagacagaggccagtcttggccactcaccccacaccccgcggaccccaataaaggcttgcatagctcca

SEQ ID NO: 316 - DNA, 294 bp, synthetic construct
  source 1..294: mol_type = other DNA
aagtggcgctttctccgccccaggtctttccccctcttttctagggttccaggacatagtctgaggcaagatggagggtatgaggggccttcacacttcacttcatcccttctacccatcacaacatacaaagcaactacacctggatttttccaaacaacttttatttcctcagagtcttccttaatcctatggaacaagaagctgccactgaatagggcccagtataggggcttgcttttctactccctccccccaatataaaaatatagacttttttttgtggtcccaaaa

SEQ ID NO: 317 - DNA, 175 bp, Homo sapiens
  source 1..175: mol_type = genomic DNA
gcactcccagcccggggcctgctgcctccagcagccacttcagagcccccgcctttgcctgcactcctcttgcagggctggccctgcctgctcctgcggcagcctctggtgacgtgctgtccaccaggccttggagacaggctagcctggccacagaattaaacgtgttgccaca

SEQ ID NO: 318 - DNA, 59 bp, Homo sapiens
  source 1..59: mol_type = genomic DNA
cctgagcggttaccaccagccccaggcctgcggaggcgctagtccaccagagcccctcc

SEQ ID NO: 319 - DNA, 120 bp, Homo sapiens
  source 1..120: mol_type = genomic DNA
ggccctgggccagattacttcctccacccctccctatctcacctgcccagccctgtgctggggccctgcaattggaatgttggccagatttctgcaataaacacttgtggtttgcggcca

SEQ ID NO: 320 - DNA, 165 bp, synthetic construct
  source 1..165: mol_type = other DNA
ggtgtcttctgggacctgccggcctcccgggccagctgccccacccctgcccatgcctgtcctgcacggctctgctgctcgggcccacagcgccgtcccatcacaagcccggggagggatcccgcctttgaaaataaagctgttatgggtgtcattcaggaaata

SEQ ID NO: 321 - DNA, 79 bp, Homo sapiens
  source 1..79: mol_type = genomic DNA
aacctcccactttgtctgtacatactggcctctgtgattacatagatcagccattaaaataaaacaagccttaatctgc

SEQ ID NO: 322 - DNA, 255 bp, Homo sapiens
  source 1..255: mol_type = genomic DNA
gcccctcccctgccctctccctgaaataaagaacagcttgacagaagccctggctctcctgctgtccgtgggtgggtgtgggtgtgtcgggggcccgcagtcccctgtctggtgcccgctctgagccacaccctctccgggtgctgcctggtcgtgaatcaaaagccgtggcccgcccacccttcccggggcagcaggtgaggaagccgccgtactgcaaatgactttaatcattaaatagcttctatgccacac

SEQ ID NO: 323 - DNA, 87 bp, synthetic construct
  source 1..87: mol_type = other DNA
tcaccaaaaagcaaccaacttagccagttttatttgcaaaacaaggaaataaaggcttacttctttaaaaagtctctggactcttaa

SEQ ID NO: 324 - DNA, 54 bp, Homo sapiens
  source 1..54: mol_type = genomic DNA
ggctggacatcggcccgctccccacaatgaaataaagttattttctcattccca

SEQ ID NO: 325 - DNA, 114 bp, Homo sapiens
  source 1..114: mol_type = genomic DNA
atctgctggcagagtagtttgagccagctggactgggctggccgcctgccgcttcttgagggtggaagaggggtgctctgagaagacactcaggcagcagactctgcctctcac

SEQ ID NO: 326 - DNA, 72 bp, Homo sapiens
  source 1..72: mol_type = genomic DNA
gggcggcaccccagccctgcccttgcttccttcaataaacatcacaggacctgggactgcacaggacctggg

SEQ ID NO: 327 - DNA, 283 bp, Homo sapiens
  source 1..283: mol_type = genomic DNA
gcacccaagggtaacaaccagatggcttcactgaaacctgcttttgtaaattacttttttttactgttgctggaagtgtcccacctgctgctcataataaatgcagatgtatagcagtcctgtctggggcccattctgcaggggtggtgggtgctgggacctacattgcttccccaatctcttccctttcccatttctcatctgcctttcacagttcctgctgctgatctacttaagggggatgagatttgtgtagcaccggtgttttgtcattggaatttaa

SEQ ID NO: 328 - DNA, 264 bp, Homo sapiens
  source 1..264: mol_type = genomic DNA
gcctgcctcgcacctgctgagccgcactgctgcccctggcctcaccggcctcaggagacggtggggccgctgcatttcggggtggtcttgggaatcccaggccctaggggtcacatttgctcaggaagtgggtatcaaattgaggtgggggtgtcagaggaggcaaaggggtcccagctgcggtcaggactgtggtgggattcccgcctcggcagcgtcactgcccagtagctgctcaataaaggctgtgtgtgtggaatctga

SEQ ID NO: 329 - DNA, 27 bp, Homo sapiens
  source 1..27: mol_type = genomic DNA
tttcccctcttcccttcaacatgaaaa

SEQ ID NO: 330 - DNA, 234 bp, Homo sapiens
  source 1..234: mol_type = genomic DNA
gcgcgcccgctgaacctcccgctgctgctgctgctgctgggggccactgtggccgccgaactcatctcctgcctgcaggccccaaggtccaccctgtctggccacaggcaccgcctccatcccatgtcccgcccagccccgcccccaacccaaggtgctgagagatctccagctgcacaggccaccgccccagggcgtggccgctgttacagaaacaataaaccctgatgggca

SEQ ID NO: 331 - DNA, 170 bp, Homo sapiens
  source 1..170: mol_type = genomic DNA
gagatgctggcctgcgcctgcacctgcgcctggctctgtcaccgccatgcaactccatgcctatttactggaaacctgttatgccaaacagttgtaccactgctaataaatgaccagtttacctgaaaccctttgtgatcagttctttaatgatacctaaatgaaagcta

SEQ ID NO: 332 - DNA, 478 bp, Homo sapiens
  source 1..478: mol_type = genomic DNA
ggcagcctcacagcctgccatctggtgcctcctgccacctggtgcctctcggctcagtgacagccaacctgccccctccccacaccaatcagccaggctgagcccccacccctgccccagctccaggacctgcccctgagccgggccttctagtcgtagtgccttcagggtccgaggagcatcaggctcctgcagagccccatccccccgccacacccacacggtggagctgcctcttccttcccctcctccctgttgcccatactcagcatctcggatgaaagggctcccttgtcctcaggctccacgggagcggggctgctggagagagcggggaactcccaccacagtggggcatccggcactgaagccctggtgttcctggtcacgtcccccaggggaccctgcccacttcctggacttcgtgccttactgagtctctaagactttttctaataaacaagccagtgcgtgtacc

SEQ ID NO: 333 - DNA, 251 bp, Homo sapiens
  source 1..251: mol_type = genomic DNA
gtgcccggcccaccagggactgcagctgcaccagcaggtgccatcacgcttgtgatgtggtggccgtcacgctttcatgaccactgggcctgctagtctgtcagggccttggcccaggggtcagcagagcttcagaggtggccccacctgagcccccacccgggagcagtgtcctgtgctttctgcatgcttagagcatgttcttggaacatggaattttataagctgaataaagtttttgacttccttta

SEQ ID NO: 334 - DNA, 36 bp, Homo sapiens
  source 1..36: mol_type = genomic DNA
cgcgcctagcagtgtcccagccgggttcgtgtcgcc

SEQ ID NO: 335 - RNA, 120 nt, synthetic construct
  source 1..120: mol_type = other RNA
aaaaaaataaaaaaaaattaaaaaaaaaatttaaaaaaaaattaaaaaaatttaaaaaaaattaaaaaaaaaataaaaaattaaaaaaataaaaaaaaaatttaaaaaaaaataaaaaaa

SEQ ID NO: 336 - DNA, 120 bp, synthetic construct
  source 1..120: mol_type = other DNA
aaaaaaataaaaaaaaattaaaaaaaaaatttaaaaaaaaattaaaaaaatttaaaaaaaattaaaaaaaaaataaaaaattaaaaaaataaaaaaaaaatttaaaaaaaaataaaaaaa

SEQ ID NO: 337 - RNA, 120 nt, synthetic construct
  source 1..120: mol_type = other RNA
aaaaaaacaaaaaaaaaccaaaaaaaaaacccaaaaaaaaaccaaaaaaacccaaaaaaaaccaaaaaaaaaacaaaaaaccaaaaaaacaaaaaaaaaacccaaaaaaaaacaaaaaaa

SEQ ID NO: 338 - RNA, 50 nt, synthetic construct
  source 1..50: mol_type = other RNA
aaaaaaacaaaaaaaaaccaaaaaaaaaacccaaaaaaaaaccaaaaaaa